SEQUENCE LISTING (594 sequences)

SEQ ID NO: 1 - protein, 5 aa, synthetic construct
KYTMH

SEQ ID NO: 2 - protein, 17 aa, synthetic construct
HFNPNNDETKYNQKFTG

SEQ ID NO: 3 - protein, 10 aa, synthetic construct
DGLGLRLFAD

SEQ ID NO: 4 - protein, 11 aa, synthetic construct
GASENIYGALN

SEQ ID NO: 5 - protein, 7 aa, synthetic construct
GATNLAD

SEQ ID NO: 6 - protein, 9 aa, synthetic construct
QNILDTPWT

SEQ ID NO: 7 - protein, 5 aa, synthetic construct
KYAIS

SEQ ID NO: 8 - protein, 17 aa, synthetic construct
HFNPNNDETKYNQKFQG

SEQ ID NO: 9 - protein, 10 aa, synthetic construct
DGLGLRLFAD

SEQ ID NO: 10 - protein, 11 aa, synthetic construct
RASENIYGALN

SEQ ID NO: 11 - protein, 7 aa, synthetic construct
GATNLAD

SEQ ID NO: 12 - protein, 9 aa, synthetic construct
QNILDTPWT

SEQ ID NO: 13 - protein, 5 aa, synthetic construct
NFAIS

SEQ ID NO: 14 - protein, 17 aa, synthetic construct
GIIPGHAKANYAQKFQG

SEQ ID NO: 15 - protein, 10 aa, synthetic construct
DGLGIRLFAD

SEQ ID NO: 16 - protein, 11 aa, synthetic construct
RASQEIYGALN

SEQ ID NO: 17 - protein, 7 aa, synthetic construct
GATNLQS

SEQ ID NO: 18 - protein, 9 aa, synthetic construct
QDIYDAPWT

SEQ ID NO: 19 - protein, 5 aa, synthetic construct
KFAIS

SEQ ID NO: 20 - protein, 17 aa, synthetic construct
GIIPGHAKANYAQKFQG

SEQ ID NO: 21 - protein, 10 aa, synthetic construct
DGLGIRLFAD

SEQ ID NO: 22 - protein, 11 aa, synthetic construct
RASQEIYGALN

SEQ ID NO: 23 - protein, 7 aa, synthetic construct
GATNLQS

SEQ ID NO: 24 - protein, 9 aa, synthetic construct
QDIYDAPWT

SEQ ID NO: 25 - protein, 5 aa, synthetic construct
KYAIS

SEQ ID NO: 26 - protein, 17 aa, synthetic construct
GIIPGHAKANYAQKFQG

SEQ ID NO: 27 - protein, 10 aa, synthetic construct
DGLGIRLFAD

SEQ ID NO: 28 - protein, 11 aa, synthetic construct
RASQKIYGALN

SEQ ID NO: 29 - protein, 7 aa, synthetic construct
GATNLQS

SEQ ID NO: 30 - protein, 9 aa, synthetic construct
QNTYDTPWT

SEQ ID NO: 31 - protein, 5 aa, synthetic construct
GHAIS

SEQ ID NO: 32 - protein, 17 aa, synthetic construct
GIIPGHAKANYAQKFQG

SEQ ID NO: 33 - protein, 10 aa, synthetic construct
DGLGIRLFAD

SEQ ID NO: 34 - protein, 11 aa, synthetic construct
RASQKIYGALN

SEQ ID NO: 35 - protein, 7 aa, synthetic construct
GATNLQS

SEQ ID NO: 36 - protein, 9 aa, synthetic construct
QNTYDTPWT

SEQ ID NO: 37 - protein, 5 aa, synthetic construct
DYGMS

SEQ ID NO: 38 - protein, 17 aa, synthetic construct
DINWSGEITAYADSVKG

SEQ ID NO: 39 - protein, 12 aa, synthetic construct
SNSYRWDDALDI

SEQ ID NO: 40 - protein, 11 aa, synthetic construct
RASQSVSSNLA

SEQ ID NO: 41 - protein, 7 aa, synthetic construct
GASSRAT

SEQ ID NO: 42 - protein, 10 aa, synthetic construct
QQYGSSPPVT

SEQ ID NO: 43 - protein, 5 aa, synthetic construct
DYAMH

SEQ ID NO: 44 - protein, 17 aa, synthetic construct
VISYDGSNKYYADSVKG

SEQ ID NO: 45 - protein, 13 aa, synthetic construct
DRIGWYDYDAFDI

SEQ ID NO: 46 - protein, 11 aa, synthetic construct
RASHSVGSNLA

SEQ ID NO: 47 - protein, 7 aa, synthetic construct
DASNRAT

SEQ ID NO: 48 - protein, 9 aa, synthetic construct
QQRSNWPPT

SEQ ID NO: 49 - protein, 7 aa, synthetic construct
GYTFTKY

SEQ ID NO: 50 - protein, 6 aa, synthetic construct
NPNNDE

SEQ ID NO: 51 - protein, 7 aa, synthetic construct
GYRFHNF

SEQ ID NO: 52 - protein, 6 aa, synthetic construct
IPGHAK

SEQ ID NO: 53 - protein, 7 aa, synthetic construct
GSRFYKF

SEQ ID NO: 54 - protein, 7 aa, synthetic construct
GSGFRGH

SEQ ID NO: 55 - protein, 7 aa, synthetic construct
GFTFDDY

SEQ ID NO: 56 - protein, 6 aa, synthetic construct
NWSGEI

SEQ ID NO: 57 - protein, 6 aa, synthetic construct
SYDGSN

SEQ ID NO: 58 - protein, 119 aa, synthetic construct
QVHLQQSGPELVKPGASVKMSCKTSGYTFTKYTMHWVKQGHEESLEWIGHFNPNNDETKYNQKFTGKATLTVDKSSTTAYMELRSLTSDDSALYYCARDGLGLRLFADWGQGTLITVSA

SEQ ID NO: 59 - protein, 107 aa, synthetic construct
DIQMTQSPASLSASVGETVTITCGASENIYGALNWYQRKQGKSPQLLIFGATNLADGVSSRFSGSGSDRQYSLKISSLHPDDVATYYCQNILDTPWTFGGGTKLEIK

SEQ ID NO: 60 - protein, 119 aa, synthetic construct
QVQLVQSGAEVKKPGSSVKVSCKASGYTFTKYAISWVRQAPGQGLEWMGHFNPNNDETKYNQKFQGRVTITADESTSTAYMELSSLRSEDTAVYYCARDGLGLRLFADWGQGTLVTVSS

SEQ ID NO: 61 - protein, 107 aa, synthetic construct
DIQMTQSPSSLSASVGDRVTITCRASENIYGALNWYQQKPGKAPKLLIYGATNLADGVPSRFSGSGSGTDFTLTISSLQPEDFATYYCQNILDTPWTFGGGTKLEIK

SEQ ID NO: 62 - protein, 119 aa, synthetic construct
QVQLVQSGAEVKKPGSSVKVSCKASGYRFHNFAISWVRQAPGQGLEWMGGIIPGHAKANYAQKFQGRVTITADESTSTAYMELSSLRSEDTAVYYCARDGLGIRLFADWGQGTLVTVSS

SEQ ID NO: 63 - protein, 107 aa, synthetic construct
DIQMTQSPSSLSASVGDRVTITCRASQEIYGALNWYQQKPGKAPKLLIYGATNLQSGVPSRFSGSGSGTDFTLTISSLQPEDFATYYCQDIYDAPWTFGGGTKVEIK

SEQ ID NO: 64 - protein, 119 aa, synthetic construct
QVQLVQSGAEVKKPGSSVKVSCKASGSRFYKFAISWVRQAPGQGLEWMGGIIPGHAKANYAQKFQGRVTITADESTSTAYMELSSLRSEDTAVYYCARDGLGIRLFADWGQGTLVTVSS

SEQ ID NO: 65 - protein, 107 aa, synthetic construct; identical to SEQ ID NO: 63

SEQ ID NO: 66 - protein, 119 aa, synthetic construct
QVQLVQSGAEVKKPGSSVKVSCKASGYTFTKYAISWVRQAPGQGLEWMGGIIPGHAKANYAQKFQGRVTITADESTSTAYMELSSLRSEDTAVYYCARDGLGIRLFADWGQGTLVTVSS

SEQ ID NO: 67 - protein, 107 aa, synthetic construct
DIQMTQSPSSLSASVGDRVTITCRASQKIYGALNWYQQKPGKAPKLLIYGATNLQSGVPSRFSGSGSGTDFTLTISSLQPEDFATYYCQNTYDTPWTFGGGTKVEIK

SEQ ID NO: 68 - protein, 119 aa, synthetic construct
QVQLVQSGAEVKKPGSSVKVSCKASGSGFRGHAISWVRQAPGQGLEWMGGIIPGHAKANYAQKFQGRVTITADESTSTAYMELSSLRSEDTAVYYCARDGLGIRLFADWGQGTLVTVSS

SEQ ID NO: 69 - protein, 107 aa, synthetic construct; identical to SEQ ID NO: 67

SEQ ID NO: 70 - protein, 121 aa, synthetic construct
EVQLVESGGGAVRPGGSLRLSCAASGFTFDDYGMSWVRQAPGKGLEWVSDINWSGEITAYADSVKGRFTISRDNAKNSLYLQMNSLRAEDTAVYYCARSNSYRWDDALDIWGQGTMVTVSS

SEQ ID NO: 71 - protein, 108 aa, synthetic construct
EIVLTQSPATLSVSPGERATLSCRASQSVSSNLAWYQQKPGQAPRLLIYGASSRATGIPDRFSGSGSGTDFTLTISRLEPEDFAVYYCQQYGSSPPVTFGQGTKVEIK

SEQ ID NO: 72 - protein, 122 aa, synthetic construct
EVQLVESGGGLVQPGRSLRLSCAASGFTFDDYAMHWVRQAPGKGLEWVAVISYDGSNKYYADSVKGRFTISRDNSKNTLYLQMNSLRAEDTAVYYCAKDRIGWYDYDAFDIWGQGTMVTVSS

SEQ ID NO: 73 - protein, 107 aa, synthetic construct
EIVLTQSPATLSVTPGEGATLSCRASHSVGSNLAWYQQKPGQAPRLLIYDASNRATGIPARFSGSGSGTDFTLTISSLEPEDLAVYYCQQRSNWPPTFGQGTRLEIK

SEQ ID NO: 74 - protein, 51 aa, synthetic construct
  VARIANT 5..48: note = GGGS repeats may be deleted
  VARIANT 50..51: note = Residue may be deleted
GGGSGGGSGGGSGGGSGGGSGGGSGGGSGGGSGGGSGGGSGGGSGGGSGGG

SEQ ID NO: 75 - protein, 63 aa, synthetic construct
  VARIANT 6..60: note = GGGGS repeats may be deleted
  VARIANT 62..63: note = Residue may be deleted
GGGGSGGGGSGGGGSGGGGSGGGGSGGGGSGGGGSGGGGSGGGGSGGGGSGGGGSGGGGSGGG

SEQ ID NO: 76 - protein, 75 aa, synthetic construct
  VARIANT 7..72: note = GGGGGS repeats may be deleted
  VARIANT 74..75: note = Residue may be deleted
GGGGGSGGGGGSGGGGGSGGGGGSGGGGGSGGGGGSGGGGGSGGGGGSGGGGGSGGGGGSGGGGGSGGGGGSGGG

SEQ ID NO: 77 - protein, 20 aa, synthetic construct
  VARIANT 11..20: note = GGGGS repeats may be deleted
GGGGSGGGGSGGGGSGGGGS

SEQ ID NO: 78 - protein, 15 aa, synthetic construct
GGGGSGGGGSGGGGS

SEQ ID NO: 79 - protein, 15 aa, synthetic construct
GGGGSGGGGSGGGGS

SEQ ID NO: 80 - protein, 133 aa, synthetic construct
APTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 81 - protein, 133 aa, Homo sapiens [sequence omitted]

SEQ ID NO: 82 - protein, 272 aa, synthetic construct
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 83 - protein, 551 aa, synthetic construct
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 84 - protein, 369 aa, synthetic construct
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 85 - protein, 133 aa, synthetic construct
APTSSSTKKTQLQLEHLLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 86 - protein, 133 aa, synthetic construct
APTSSSTKKTQLQLEHLLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 87 - protein, 133 aa, synthetic construct
APTSSSTKKTQLQLEHLLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFCQSIISTLT

SEQ ID NO: 88 - protein, 133 aa, synthetic construct
APTSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 89 - protein, 133 aa, synthetic construct
APTSSSTKKTQLQLEHLLLDLQMILNGINNYKNPKLTEMLTAKFYMPKKATELKHLQCLEEELKPLEEVLNLAQSKNFHLRPRDLISSINVIVLELKGSETTFMCEYADETATIVEFLNRWITFCQSIISTLT

SEQ ID NO: 90 - protein, 133 aa, synthetic construct
APTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFCQSIISTLT

SEQ ID NO: 91 - protein, 133 aa, synthetic construct
APTSSSTKKTQLQLEHLLLDLQMILNGINNYKNPKLTEMLTAKFYMPKKATELKHLQCLEEELKPLEEVLNLAQSKNFHLRPRDLISNINEIVLELKGSETTFMCEYADETATIVEFLNRWITFCQSIISTLT

SEQ ID NO: 92 - protein, 133 aa, synthetic construct
APTSSSTKKTQLQLEHLLLDLQMILNGINNYKNPKLTEMLTAKFYMPKKATELKHLQCLEEELKPLEEVLNLAQSKNFHLRPRHLISNINVIVLELKGSETTFMCEYADETATIVEFLNRWITFCQSIISTLT

SEQ ID NO: 93 - protein, 133 aa, synthetic construct
APTSSSTKKTQLQLEDLLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 94 - protein, 133 aa, synthetic construct
APTSSSTKKTQLQLEELLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 95 - protein, 133 aa, synthetic construct
APTSSSTKKTQLQLEHLLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFCSSIISTLT

SEQ ID NO: 96 - protein, 133 aa, synthetic construct
APTSSSTKKTQLQLEHLLLDLQMILNGINNYKNPKLTDMLTAKFYMPKKATELKHLQCLEEELKPLEEVLNLAQSKNFHLRPRDLISSINVIVLELKGSETTFMCEYADETATIVEFLNRWITFCQSIISTLT

SEQ ID NO: 97 - protein, 133 aa, synthetic construct
APTSSSTKKTQLQLEHLLLDLQMILNGINNYKNPKLTDMLTAKFYMPKKATELKHLQCLEEELKPLEEVLNLAQSKNFHLRPRDLISAINVIVLELKGSETTFMCEYADETATIVEFLNRWITFCQSIISTLT

SEQ ID NO: 98 - protein, 133 aa, synthetic construct
APTSSSTKKTQLQLEHLLLDLQMILNGINNYKNPKLTDMLTAKFYMPKKATELKHLQCLEEELKPLEEVLNLAQSKNFHLRPRDLISNINEIVLELKGSETTFMCEYADETATIVEFLNRWITFCQSIISTLT

SEQ ID NO: 99 - protein, 133 aa, synthetic construct
APTSSSTKKTQLQLEHLLLDLQMILNGINNYKNPKLTDMLTAKFYMPKKATELKHLQCLEEELKPLEEVLNLAQSKNFHLRPRHLISNINVIVLELKGSETTFMCEYADETATIVEFLNRWITFCQSIISTLT

SEQ ID NO: 100 - protein, 133 aa, synthetic construct
APTSSSTKKTQLQLEDLLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 101 - protein, 133 aa, synthetic construct
APTSSSTKKTQLQLEELLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 102 - protein, 133 aa, synthetic construct
APTSSSTKKTQLQLEHLLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFCSSIISTLT

SEQ ID NO: 103 - protein, 133 aa, synthetic construct
APTSSSTKKTQLQLEHLLLDLQMILNGINNYKNPKLTAMLTKKFYMPKKATELKHLQCLEEELKPLEEVLNLAQSKNFHLRPRDLISSINVIVLELKGSETTFMCEYADETATIVEFLNRWITFCQSIISTLT

SEQ ID NO: 104 - protein, 133 aa, synthetic construct
APTSSSTKKTQLQLEHLLLDLQMILNGINNYKNPKLTAMLTKKFYMPKKATELKHLQCLEEELKPLEEVLNLAQSKNFHLRPRDLISAINVIVLELKGSETTFMCEYADETATIVEFLNRWITFCQSIISTLT

SEQ ID NO: 105 - protein, 133 aa, synthetic construct
APTSSSTKKTQLQLEHLLLDLQMILNGINNYKNPKLTAMLTKKFYMPKKATELKHLQCLEEELKPLEEVLNLAQSKNFHLRPRDLISNINEIVLELKGSETTFMCEYADETATIVEFLNRWITFCQSIISTLT

SEQ ID NO: 106 - protein, 133 aa, synthetic construct
APTSSSTKKTQLQLEHLLLDLQMILNGINNYKNPKLTAMLTKKFYMPKKATELKHLQCLEEELKPLEEVLNLAQSKNFHLRPRHLISNINVIVLELKGSETTFMCEYADETATIVEFLNRWITFCQSIISTLT

SEQ ID NO: 107 - protein, 133 aa, synthetic construct
APTSSSTKKTQLQLEDLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 108 - protein, 133 aa, synthetic construct
APTSSSTKKTQLQLEELLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 109 - protein, 133 aa, synthetic construct
APTSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFCSSIISTLT

SEQ ID NO: 110 - protein, 133 aa, synthetic construct
APTSSSTKKTQLQLEHLLLDLQMILNGINNYKNPKLTRMLTAKFYMPKKATELKHLQCLEEQLKPLEEVLNLAQSKNFHLRPRDLISSINVIVLELKGSETTFMCEYADETATIVEFLNRWITFCQSIISTLT

SEQ ID NO: 111 - protein, 133 aa, synthetic construct
APTSSSTKKTQLQLEHLLLDLQMILNGINNYKNPKLTRMLTAKFYMPKKATELKHLQCLEEQLKPLEEVLNLAQSKNFHLRPRDLISAINVIVLELKGSETTFMCEYADETATIVEFLNRWITFCQSIISTLT

SEQ ID NO: 112 - protein, 133 aa, synthetic construct
APTSSSTKKTQLQLEHLLLDLQMILNGINNYKNPKLTRMLTAKFYMPKKATELKHLQCLEEQLKPLEEVLNLAQSKNFHLRPRDLISNINEIVLELKGSETTFMCEYADETATIVEFLNRWITFCQSIISTLT

SEQ ID NO: 113 - protein, 133 aa, synthetic construct
APTSSSTKKTQLQLEHLLLDLQMILNGINNYKNPKLTRMLTAKFYMPKKATELKHLQCLEEQLKPLEEVLNLAQSKNFHLRPRHLISNINVIVLELKGSETTFMCEYADETATIVEFLNRWITFCQSIISTLT

SEQ ID NO: 114 - protein, 133 aa, synthetic construct
APTSSSTKKTQLQLEDLLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFCQSIISTLT

SEQ ID NO: 115 - protein, 133 aa, synthetic construct
APTSSSTKKTQLQLEELLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFCQSIISTLT

SEQ ID NO: 116 - protein, 133 aa, synthetic construct
APTSSSTKKTQLQLEHLLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFCSSIISTLT

SEQ ID NO: 117 - protein, 133 aa, synthetic construct
APTSSSTKKTQLQLEHLLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 118 - protein, 133 aa, synthetic construct
APTSSSTKKTQLQLEHLLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 119 - protein, 133 aa, synthetic construct
APTSSSTKKTQLQLEHLLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFAQSIISTLT

SEQ ID NO: 120 - protein, 133 aa, synthetic construct
APTSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 121 - protein, 133 aa, synthetic construct
APTSSSTKKTQLQLEHLLLDLQMILNGINNYKNPKLTEMLTAKFYMPKKATELKHLQCLEEELKPLEEVLNLAQSKNFHLRPRDLISSINVIVLELKGSETTFMCEYADETATIVEFLNRWITFAQSIISTLT

SEQ ID NO: 122 - protein, 133 aa, synthetic construct; identical to SEQ ID NO: 80

SEQ ID NO: 123 - protein, 133 aa, synthetic construct
APTSSSTKKTQLQLEHLLLDLQMILNGINNYKNPKLTEMLTAKFYMPKKATELKHLQCLEEELKPLEEVLNLAQSKNFHLRPRDLISNINEIVLELKGSETTFMCEYADETATIVEFLNRWITFAQSIISTLT

SEQ ID NO: 124 - protein, 133 aa, synthetic construct
APTSSSTKKTQLQLEHLLLDLQMILNGINNYKNPKLTEMLTAKFYMPKKATELKHLQCLEEELKPLEEVLNLAQSKNFHLRPRHLISNINVIVLELKGSETTFMCEYADETATIVEFLNRWITFAQSIISTLT

SEQ ID NO: 125 - protein, 133 aa, synthetic construct
APTSSSTKKTQLQLEDLLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 126 - protein, 133 aa, synthetic construct
APTSSSTKKTQLQLEELLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 127 - protein, 133 aa, synthetic construct
APTSSSTKKTQLQLEHLLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFASSIISTLT

SEQ ID NO: 128 - protein, 133 aa, synthetic construct
APTSSSTKKTQLQLEHLLLDLQMILNGINNYKNPKLTDMLTAKFYMPKKATELKHLQCLEEELKPLEEVLNLAQSKNFHLRPRDLISSINVIVLELKGSETTFMCEYADETATIVEFLNRWITFAQSIISTLT

SEQ ID NO: 129 - protein, 133 aa, synthetic construct
APTSSSTKKTQLQLEHLLLDLQMILNGINNYKNPKLTDMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 130 - protein, 133 aa, synthetic construct
APTSSSTKKTQLQLEHLLLDLQMILNGINNYKNPKLTDMLTAKFYMPKKATELKHLQCLEEELKPLEEVLNLAQSKNFHLRPRDLISNINEIVLELKGSETTFMCEYADETATIVEFLNRWITFAQSIISTLT

SEQ ID NO: 131 - protein, 133 aa, synthetic construct
APTSSSTKKTQLQLEHLLLDLQMILNGINNYKNPKLTDMLTAKFYMPKKATELKHLQCLEEELKPLEEVLNLAQSKNFHLRPRHLISNINVIVLELKGSETTFMCEYADETATIVEFLNRWITFAQSIISTLT

SEQ ID NO: 132 - protein, 133 aa, synthetic construct
APTSSSTKKTQLQLEDLLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 133 - protein, 133 aa, synthetic construct
APTSSSTKKTQLQLEELLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 134 - protein, 133 aa, synthetic construct
APTSSSTKKTQLQLEHLLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFASSIISTLT

SEQ ID NO: 135 - protein, 133 aa, synthetic construct
APTSSSTKKTQLQLEHLLLDLQMILNGINNYKNPKLTAMLTKKFYMPKKATELKHLQCLEEELKPLEEVLNLAQSKNFHLRPRDLISSINVIVLELKGSETTFMCEYADETATIVEFLNRWITFAQSIISTLT

SEQ ID NO: 136 - protein, 133 aa, synthetic construct
APTSSSTKKTQLQLEHLLLDLQMILNGINNYKNPKLTAMLTKKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 137 - protein, 133 aa, synthetic construct
APTSSSTKKTQLQLEHLLLDLQMILNGINNYKNPKLTAMLTKKFYMPKKATELKHLQCLEEELKPLEEVLNLAQSKNFHLRPRDLISNINEIVLELKGSETTFMCEYADETATIVEFLNRWITFAQSIISTLT

SEQ ID NO: 138 - protein, 133 aa, synthetic construct
APTSSSTKKTQLQLEHLLLDLQMILNGINNYKNPKLTAMLTKKFYMPKKATELKHLQCLEEELKPLEEVLNLAQSKNFHLRPRHLISNINVIVLELKGSETTFMCEYADETATIVEFLNRWITFAQSIISTLT

SEQ ID NO: 139 - protein, 133 aa, synthetic construct
APTSSSTKKTQLQLEDLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 140 - protein, 133 aa, synthetic construct
APTSSSTKKTQLQLEELLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 141 - protein, 133 aa, synthetic construct
APTSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFASSIISTLT

SEQ ID NO: 142 - protein, 133 aa, synthetic construct
APTSSSTKKTQLQLEHLLLDLQMILNGINNYKNPKLTRMLTAKFYMPKKATELKHLQCLEEQLKPLEEVLNLAQSKNFHLRPRDLISSINVIVLELKGSETTFMCEYADETATIVEFLNRWITFAQSIISTLT

SEQ ID NO: 143 - protein, 133 aa, synthetic construct
APTSSSTKKTQLQLEHLLLDLQMILNGINNYKNPKLTRMLTAKFYMPKKATELKHLQCLEEQLKPLEEVLNLAQSKNFHLRPRDLISAINVIVLELKGSETTFMCEYADETATIVEFLNRWITFAQSIISTLT

SEQ ID NO: 144 - protein, 133 aa, synthetic construct
APTSSSTKKTQLQLEHLLLDLQMILNGINNYKNPKLTRMLTAKFYMPKKATELKHLQCLEEQLKPLEEVLNLAQSKNFHLRPRDLISNINEIVLELKGSETTFMCEYADETATIVEFLNRWITFAQSIISTLT

SEQ ID NO: 145 - protein, 133 aa, synthetic construct
APTSSSTKKTQLQLEHLLLDLQMILNGINNYKNPKLTRMLTAKFYMPKKATELKHLQCLEEQLKPLEEVLNLAQSKNFHLRPRHLISNINVIVLELKGSETTFMCEYADETATIVEFLNRWITFAQSIISTLT

SEQ ID NO: 146 - protein, 133 aa, synthetic construct
APTSSSTKKTQLQLEDLLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFAQSIISTLT

SEQ ID NO: 147 - protein, 133 aa, synthetic construct
APTSSSTKKTQLQLEELLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFAQSIISTLT

SEQ ID NO: 148 - protein, 133 aa, synthetic construct
APTSSSTKKTQLQLEHLLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFASSIISTLT

SEQ ID NO: 149 - protein, 133 aa, synthetic construct
APTSSSTKKTQLQLEHLLLDLQMILNGINNYKNPKLTRMLTAKFYMPKKATELKHLQCLEEELKPLEEVLNLAQSKNFHLRPRDLISSINVIVLELKGSETTFMCEYADETATIVEFLNRWITFAQSIISTLT

SEQ ID NO: 150 - protein, 133 aa, synthetic construct
APTSSSTKKTQLQLEHLLLDLQMILNGINNYKNPKLTRMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 151 - protein, 133 aa, synthetic construct
APTSSSTKKTQLQLEHLLLDLQMILNGINNYKNPKLTRMLTAKFYMPKKATELKHLQCLEEELKPLEEVLNLAQSKNFHLRPRDLISNINEIVLELKGSETTFMCEYADETATIVEFLNRWITFAQSIISTLT

SEQ ID NO: 152 - protein, 133 aa, synthetic construct
APTSSSTKKTQLQLEHLLLDLQMILNGINNYKNPKLTRMLTAKFYMPKKATELKHLQCLEEELKPLEEVLNLAQSKNFHLRPRHLISNINVIVLELKGSETTFMCEYADETATIVEFLNRWITFAQSIISTLT

SEQ ID NO: 153 - protein, 133 aa, synthetic construct
APTSSSTKKTQLQLEDLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 154 - protein, 133 aa, synthetic construct
APTSSSTKKTQLQLEELLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 155 - protein, 133 aa, synthetic construct
APTSSSTKKTQLQLEHLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFASSIISTLT

SEQ ID NO: 156 - protein, 214 aa, synthetic construct
DIQMTQSPASLSASVGETVTITCGASENIYGALNWYQRKQGKSPQLLIFGATNLADGVSSRFSGSGSDRQYSLKISSLHPDDVATYYCQNILDTPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 157 - protein, 597 aa, synthetic construct
QVHLQQSGPELVKPGASVKMSCKTSGYTFTKYTMHWVKQGHEESLEWIGHFNPNNDETKYNQKFTGKATLTVDKSSTTAYMELRSLTSDDSALYYCARDGLGLRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 158 - protein, 448 aa, synthetic construct
QVHLQQSGPELVKPGASVKMSCKTSGYTFTKYTMHWVKQGHEESLEWIGHFNPNNDETKYNQKFTGKATLTVDKSSTTAYMELRSLTSDDSALYYCARDGLGLRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 159 - protein, 214 aa, synthetic construct
DIQMTQSPSSLSASVGDRVTITCRASQEIYGALNWYQQKPGKAPKLLIYGATNLQSGVPSRFSGSGSGTDFTLTISSLQPEDFATYYCQDIYDAPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 160 - protein, 597 aa, synthetic construct
QVQLVQSGAEVKKPGSSVKVSCKASGYRFHNFAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 161 - protein, 448 aa, synthetic construct
QVQLVQSGAEVKKPGSSVKVSCKASGYRFHNFAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 162 - protein, 214 aa, synthetic construct; identical to SEQ ID NO: 159

SEQ ID NO: 163 - protein, 597 aa, synthetic construct
QVQLVQSGAEVKKPGSSVKVSCKASGSRFYKFAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 164 - protein, 448 aa, synthetic construct
QVQLVQSGAEVKKPGSSVKVSCKASGSRFYKFAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 165 - protein, 214 aa, synthetic construct
DIQMTQSPSSLSASVGDRVTITCRASQKIYGALNWYQQKPGKAPKLLIYGATNLQSGVPSRFSGSGSGTDFTLTISSLQPEDFATYYCQNTYDTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 166 - protein, 597 aa, synthetic construct
QVQLVQSGAEVKKPGSSVKVSCKASGYTFTKYAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 167 - protein, 448 aa, synthetic construct
QVQLVQSGAEVKKPGSSVKVSCKASGYTFTKYAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 168 - protein, 214 aa, synthetic construct; identical to SEQ ID NO: 165

SEQ ID NO: 169 - protein, 597 aa, synthetic construct
QVQLVQSGAEVKKPGSSVKVSCKASGSGFRGHAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 170 - protein, 448 aa, synthetic construct
QVQLVQSGAEVKKPGSSVKVSCKASGSGFRGHAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 171 - protein, 215 aa, synthetic construct
EIVLTQSPATLSVSPGERATLSCRASQSVSSNLAWYQQKPGQAPRLLIYGASSRATGIPDRFSGSGSGTDFTLTISRLEPEDFAVYYCQQYGSSPPVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 172 - protein, 599 aa, synthetic construct
EVQLVESGGGAVRPGGSLRLSCAASGFTFDDYGMSWVRQAPGKGLEWVSDINWSGEITAYADSVKGRFTISRDNAKNSLYLQMNSLRAEDTAVYYCARSNSYRWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 173 - protein, 450 aa, synthetic construct
EVQLVESGGGAVRPGGSLRLSCAASGFTFDDYGMSWVRQAPGKGLEWVSDINWSGEITAYADSVKGRFTISRDNAKNSLYLQMNSLRAEDTAVYYCARSNSYRWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 174 - protein, 214 aa, synthetic construct
EIVLTQSPATLSVTPGEGATLSCRASHSVGSNLAWYQQKPGQAPRLLIYDASNRATGIPARFSGSGSGTDFTLTISSLEPEDLAVYYCQQRSNWPPTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 175 - protein, 600 aa, synthetic construct
EVQLVESGGGLVQPGRSLRLSCAASGFTFDDYAMHWVRQAPGKGLEWVAVISYDGSNKYYADSVKGRFTISRDNSKNTLYLQMNSLRAEDTAVYYCAKDRIGWYDYDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 176 - protein, 451 aa, synthetic construct
EVQLVESGGGLVQPGRSLRLSCAASGFTFDDYAMHWVRQAPGKGLEWVAVISYDGSNKYYADSVKGRFTISRDNSKNTLYLQMNSLRAEDTAVYYCAKDRIGWYDYDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 177 - protein, 5 aa, synthetic construct
SYWMN

SEQ ID NO: 178 - protein, 17 aa, synthetic construct
QIYPGDGDTNYNGKFKG

SEQ ID NO: 179 - protein, 13 aa, synthetic construct
SGAAFSSYYAMDY

SEQ ID NO: 180 - protein, 11 aa, synthetic construct
RASENIYSNLA

SEQ ID NO: 181 - protein, 7 aa, synthetic construct
AATNLAD

SEQ ID NO: 182 - protein, 9 aa, synthetic construct
QHFWGTPWT

SEQ ID NO: 183 - protein, 7 aa, synthetic construct
GYAFSSY

SEQ ID NO: 184 - protein, 6 aa, synthetic construct
YPGDGD

SEQ ID NO: 185 - protein, 122 aa, synthetic construct
QVQLQQSGAELVRPGSSVKISCKASGYAFSSYWMNWVKQRPGQGLEWIGQIYPGDGDTNYNGKFKGKATLTADKSSSTAYMQLSSLTSEDSAVYFCARSGAAFSSYYAMDYWGQGTSVTVSS

SEQ ID NO: 186 - protein, 107 aa, synthetic construct
DIQMTQSPASLSVSVGETVTITCRASENIYSNLAWYQQKQGKSPQLLVYAATNLADGVPSRFSGSGSGTQYSLKINSLQSEDFGSYYCQHFWGTPWTFGGGTKLEIK

SEQ ID NO: 187 - protein, 214 aa, synthetic construct
DIQMTQSPASLSVSVGETVTITCRASENIYSNLAWYQQKQGKSPQLLVYAATNLADGVPSRFSGSGSGTQYSLKINSLQSEDFGSYYCQHFWGTPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 188 - protein, 600 aa, synthetic construct
QVQLQQSGAELVRPGSSVKISCKASGYAFSSYWMNWVKQRPGQGLEWIGQIYPGDGDTNYNGKFKGKATLTADKSSSTAYMQLSSLTSEDSAVYFCARSGAAFSSYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 189 - protein, 451 aa, synthetic construct
QVQLQQSGAELVRPGSSVKISCKASGYAFSSYWMNWVKQRPGQGLEWIGQIYPGDGDTNYNGKFKGKATLTADKSSSTAYMQLSSLTSEDSAVYFCARSGAAFSSYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 190 - protein, 133 aa, synthetic construct
APTSSSTKKTQLQLEHLLLDLQMILNGINNYKNPKLTEMLTAKFYMPKKATELKHLQCLEEELKPLEEVLNLAQSKNFHLRPRDLISGINVIVLELKGSETTFMCEYADETATIVEFLNRWITFCQSIISTLT

SEQ ID NO: 191 - protein, 133 aa, synthetic construct
APTSSSTKKTQLQLEHLLLDLQMILNGINNYKNPKLTEMLTAKFYMPKKATELKHLQCLEEELKPLEEVLNLAQSKNFHLRPRKLISNINVIVLELKGSETTFMCEYADETATIVEFLNRWITFCQSIISTLT

SEQ ID NO: 192 - protein, 133 aa, synthetic construct
APTSSSTKKTQLQLEHLLLDLQMILNGINNYKNPKLTEMLTAKFYMPKKATELKHLQCLEEELKPLEEVLNLAQSKNFHLRPRRLISNINVIVLELKGSETTFMCEYADETATIVEFLNRWITFCQSIISTLT

SEQ ID NO: 193 - protein, 133 aa, synthetic construct
APTSSSTKKTQLQLEHLLLDLQMILNGINNYKNPKLTDMLTAKFYMPKKATELKHLQCLEEELKPLEEVLNLAQSKNFHLRPRDLISGINVIVLELKGSETTFMCEYADETATIVEFLNRWITFCQSIISTLT

SEQ ID NO: 194 - protein, 133 aa, synthetic construct
APTSSSTKKTQLQLEHLLLDLQMILNGINNYKNPKLTDMLTAKFYMPKKATELKHLQCLEEELKPLEEVLNLAQSKNFHLRPRKLISNINVIVLELKGSETTFMCEYADETATIVEFLNRWITFCQSIISTLT

SEQ ID NO: 195 - protein, 133 aa, synthetic construct
APTSSSTKKTQLQLEHLLLDLQMILNGINNYKNPKLTDMLTAKFYMPKKATELKHLQCLEEELKPLEEVLNLAQSKNFHLRPRRLISNINVIVLELKGSETTFMCEYADETATIVEFLNRWITFCQSIISTLT

SEQ ID NO: 196 - protein, 133 aa, synthetic construct
APTSSSTKKTQLQLEHLLLDLQMILNGINNYKNPKLTAMLTKKFYMPKKATELKHLQCLEEELKPLEEVLNLAQSKNFHLRPRDLISGINVIVLELKGSETTFMCEYADETATIVEFLNRWITFCQSIISTLT

SEQ ID NO: 197 - protein, 133 aa, synthetic construct
APTSSSTKKTQLQLEHLLLDLQMILNGINNYKNPKLTAMLTKKFYMPKKATELKHLQCLEEELKPLEEVLNLAQSKNFHLRPRKLISNINVIVLELKGSETTFMCEYADETATIVEFLNRWITFCQSIISTLT

SEQ ID NO: 198 - protein, 133 aa, synthetic construct
APTSSSTKKTQLQLEHLLLDLQMILNGINNYKNPKLTAMLTKKFYMPKKATELKHLQCLEEELKPLEEVLNLAQSKNFHLRPRRLISNINVIVLELKGSETTFMCEYADETATIVEFLNRWITFCQSIISTLT

SEQ ID NO: 199 - protein, 133 aa, synthetic construct
APTSSSTKKTQLQLEHLLLDLQMILNGINNYKNPKLTRMLTAKFYMPKKATELKHLQCLEEQLKPLEEVLNLAQSKNFHLRPRDLISGINVIVLELKGSETTFMCEYADETATIVEFLNRWITFCQSIISTLT

SEQ ID NO: 200 - protein, 133 aa, synthetic construct
APTSSSTKKTQLQLEHLLLDLQMILNGINNYKNPKLTRMLTAKFYMPKKATELKHLQCLEEQLKPLEEVLNLAQSKNFHLRPRKLISNINVIVLELKGSETTFMCEYADETATIVEFLNRWITFCQSIISTLT

SEQ ID NO: 201 - protein, 133 aa, synthetic construct
APTSSSTKKTQLQLEHLLLDLQMILNGINNYKNPKLTRMLTAKFYMPKKATELKHLQCLEEQLKPLEEVLNLAQSKNFHLRPRRLISNINVIVLELKGSETTFMCEYADETATIVEFLNRWITFCQSIISTLT

SEQ ID NO: 202 - protein, 133 aa, synthetic construct
APTSSSTKKTQLQLEHLLLDLQMILNGINNYKNPKLTEMLTAKFYMPKKATELKHLQCLEEELKPLEEVLNLAQSKNFHLRPRDLISGINVIVLELKGSETTFMCEYADETATIVEFLNRWITFAQSIISTLT

SEQ ID NO: 203 - protein, 133 aa, synthetic construct
APTSSSTKKTQLQLEHLLLDLQMILNGINNYKNPKLTEMLTAKFYMPKKATELKHLQCLEEELKPLEEVLNLAQSKNFHLRPRKLISNINVIVLELKGSETTFMCEYADETATIVEFLNRWITFAQSIISTLT

SEQ ID NO: 204 - protein, 133 aa, synthetic construct
APTSSSTKKTQLQLEHLLLDLQMILNGINNYKNPKLTEMLTAKFYMPKKATELKHLQCLEEELKPLEEVLNLAQSKNFHLRPRRLISNINVIVLELKGSETTFMCEYADETATIVEFLNRWITFAQSIISTLT

SEQ ID NO: 205 - protein, 133 aa, synthetic construct
APTSSSTKKTQLQLEHLLLDLQMILNGINNYKNPKLTDMLTAKFYMPKKATELKHLQCLEEELKPLEEVLNLAQSKNFHLRPRDLISGINVIVLELKGSETTFMCEYADETATIVEFLNRWITFAQSIISTLT

SEQ ID NO: 206 - protein, 133 aa, synthetic construct
APTSSSTKKTQLQLEHLLLDLQMILNGINNYKNPKLTDMLTAKFYMPKKATELKHLQCLEEELKPLEEVLNLAQSKNFHLRPRKLISNINVIVLELKGSETTFMCEYADETATIVEFLNRWITFAQSIISTLT

SEQ ID NO: 207 - protein, 133 aa, synthetic construct
APTSSSTKKTQLQLEHLLLDLQMILNGINNYKNPKLTDMLTAKFYMPKKATELKHLQCLEEELKPLEEVLNLAQSKNFHLRPRRLISNINVIVLELKGSETTFMCEYADETATIVEFLNRWITFAQSIISTLT

SEQ ID NO: 208 - protein, 133 aa, synthetic construct
APTSSSTKKTQLQLEHLLLDLQMILNGINNYKNPKLTAMLTKKFYMPKKATELKHLQCLEEELKPLEEVLNLAQSKNFHLRPRDLISGINVIVLELKGSETTFMCEYADETATIVEFLNRWITFAQSIISTLT

SEQ ID NO: 209 - protein, 133 aa, synthetic construct
APTSSSTKKTQLQLEHLLLDLQMILNGINNYKNPKLTAMLTKKFYMPKKATELKHLQCLEEELKPLEEVLNLAQSKNFHLRPRKLISNINVIVLELKGSETTFMCEYADETATIVEFLNRWITFAQSIISTLT

SEQ ID NO: 210 - protein, 133 aa, synthetic construct
APTSSSTKKTQLQLEHLLLDLQMILNGINNYKNPKLTAMLTKKFYMPKKATELKHLQCLEEELKPLEEVLNLAQSKNFHLRPRRLISNINVIVLELKGSETTFMCEYADETATIVEFLNRWITFAQSIISTLT

SEQ ID NO: 211 - protein, 133 aa, synthetic construct
APTSSSTKKTQLQLEHLLLDLQMILNGINNYKNPKLTRMLTAKFYMPKKATELKHLQCLEEQLKPLEEVLNLAQSKNFHLRPRDLISGINVIVLELKGSETTFMCEYADETATIVEFLNRWITFAQSIISTLT

SEQ ID NO: 212 - protein, 133 aa, synthetic construct
APTSSSTKKTQLQLEHLLLDLQMILNGINNYKNPKLTRMLTAKFYMPKKATELKHLQCLEEQLKPLEEVLNLAQSKNFHLRPRKLISNINVIVLELKGSETTFMCEYADETATIVEFLNRWITFAQSIISTLT

SEQ ID NO: 213 - protein, 133 aa, synthetic construct
APTSSSTKKTQLQLEHLLLDLQMILNGINNYKNPKLTRMLTAKFYMPKKATELKHLQCLEEQLKPLEEVLNLAQSKNFHLRPRRLISNINVIVLELKGSETTFMCEYADETATIVEFLNRWITFAQSIISTLT

SEQ ID NO: 214 - protein, 133 aa, synthetic construct
APTSSSTKKTQLQLEHLLLDLQMILNGINNYKNPKLTRMLTAKFYMPKKATELKHLQCLEEELKPLEEVLNLAQSKNFHLRPRDLISGINVIVLELKGSETTFMCEYADETATIVEFLNRWITFAQSIISTLT

SEQ ID NO: 215 - protein, 133 aa, synthetic construct
APTSSSTKKTQLQLEHLLLDLQMILNGINNYKNPKLTRMLTAKFYMPKKATELKHLQCLEEELKPLEEVLNLAQSKNFHLRPRKLISNINVIVLELKGSETTFMCEYADETATIVEFLNRWITFAQSIISTLT

SEQ ID NO: 216 - protein, 133 aa, synthetic construct
APTSSSTKKTQLQLEHLLLDLQMILNGINNYKNPKLTRMLTAKFYMPKKATELKHLQCLEEELKPLEEVLNLAQSKNFHLRPRRLISNINVIVLELKGSETTFMCEYADETATIVEFLNRWITFAQSIISTLT

SEQ ID NO: 217 - protein, 449 aa, synthetic construct
QVHLQQSGPELVKPGASVKMSCKTSGYTFTKYTMHWVKQGHEESLEWIGHFNPNNDETKYNQKFTGKATLTVDKSSTTAYMELRSLTSDDSALYYCARDGLGLRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 218 - protein, 449 aa, synthetic construct
QVQLVQSGAEVKKPGSSVKVSCKASGYRFHNFAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 219 - protein, 449 aa, synthetic construct
QVQLVQSGAEVKKPGSSVKVSCKASGSRFYKFAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 220 - protein, 449 aa, synthetic construct
QVQLVQSGAEVKKPGSSVKVSCKASGYTFTKYAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 221 - protein, 449 aa, synthetic construct
QVQLVQSGAEVKKPGSSVKVSCKASGSGFRGHAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 222 - protein, 451 aa, synthetic construct
EVQLVESGGGAVRPGGSLRLSCAASGFTFDDYGMSWVRQAPGKGLEWVSDINWSGEITAYADSVKGRFTISRDNAKNSLYLQMNSLRAEDTAVYYCARSNSYRWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 223 - protein, 452 aa, synthetic construct
EVQLVESGGGLVQPGRSLRLSCAASGFTFDDYAMHWVRQAPGKGLEWVAVISYDGSNKYYADSVKGRFTISRDNSKNTLYLQMNSLRAEDTAVYYCAKDRIGWYDYDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 224 - protein, 452 aa, synthetic construct
QVQLQQSGAELVRPGSSVKISCKASGYAFSSYWMNWVKQRPGQGLEWIGQIYPGDGDTNYNGKFKGKATLTADKSSSTAYMQLSSLTSEDSAVYFCARSGAAFSSYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 225 - protein, 5 aa, synthetic construct
SYAIS

SEQ ID NO: 226 - protein, 17 aa, synthetic construct
GIIPGAATANYAQKFQG

SEQ ID NO: 227 - protein, 10 aa, synthetic construct
DAAGIRLFAD

SEQ ID NO: 228 - protein, 9 aa, synthetic construct
QSTYDAPWT

SEQ ID NO: 229 - protein, 5 aa, synthetic construct
SYAMS

SEQ ID NO: 230 - protein, 17 aa, synthetic construct
DITYAGGSTAYADSVKG

SEQ ID NO: 231 - protein, 12 aa, synthetic construct
SNAYAWDDALDI

SEQ ID NO: 232 - protein, 17 aa, synthetic construct
GIIPGYATANYAQKFQG

SEQ ID NO: 233 - protein, 10 aa, synthetic construct
DAAGIRLFAD

SEQ ID NO: 234 - protein, 11 aa, synthetic construct
RASQSIYGALN

SEQ ID NO: 235 - protein, 7 aa, synthetic construct
GASNLQS

SEQ ID NO: 236 - protein, 9 aa, synthetic construct
QSTYTAPWT

SEQ ID NO: 237 - protein, 17 aa, synthetic construct
DISYAGGSTAYADSVKG

SEQ ID NO: 238 - protein, 7 aa, synthetic construct
GGTFSSY

SEQ ID NO: 239 - protein, 6 aa, synthetic construct
IPGAAT

SEQ ID NO: 240 - protein, 7 aa, synthetic construct
GFTFSSY

SEQ ID NO: 241 - protein, 6 aa, synthetic construct
TYAGGS

SEQ ID NO: 242 - protein, 12 aa, synthetic construct
SNAYAWDDALDI

SEQ ID NO: 243 - protein, 6 aa, synthetic construct
IPGYAT

SEQ ID NO: 244 - protein, 6 aa, synthetic construct
SYAGGS

SEQ ID NO: 245 - protein, 119 aa, synthetic construct
QVQLVQSGAEVKKPGSSVKVSCKASGGTFSSYAISWVRQAPGQGLEWMGGIIPGAATANYAQKFQGRVTITADESTSTAYMELSSLRSEDTAVYYCARDAAGIRLFADWGQGTLVTVSS

SEQ ID NO: 246 - protein, 107 aa, synthetic construct
DIQMTQSPSSLSASVGDRVTITCRASQEIYGALNWYQQKPGKAPKLLIYGATNLQSGVPSRFSGSGSGTDFTLTISSLQPEDFATYYCQSTYDAPWTFGGGTKVEIK

SEQ ID NO: 247 - protein, 121 aa, synthetic construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTMVTVSS

SEQ ID NO: 248 - protein, 108 aa, synthetic construct
EIVLTQSPGTLSLSPGERATLSCRASQSVSSNLAWYQQKPGQAPRLLIYGASSRATGIPDRFSGSGSGTDFTLTISRLEPEDFAVYYCQQYGSSPPVTFGQGTKVEIK

SEQ ID NO: 249 - protein, 121 aa, synthetic construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTLVTVSS

SEQ ID NO: 250 - protein, 108 aa, synthetic construct; identical to SEQ ID NO: 248

SEQ ID NO: 251 - protein, 119 aa, synthetic construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSS

SEQ ID NO: 252 - protein, 107 aa, synthetic construct
DIQMTQSPSSLSASVGDRVTITCRASQSIYGALNWYQQKPGKAPKLLIYGASNLQSGVPSRFSGSGSGTDFTLTISSLQPEDFATYYCQSTYTAPWTFGGGTKVEIK

SEQ ID NO: 253 - protein, 119 aa, synthetic construct; identical to SEQ ID NO: 251

SEQ ID NO: 254 - protein, 107 aa, synthetic construct; identical to SEQ ID NO: 246

SEQ ID NO: 255 - protein, 121 aa, synthetic construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTMVTVSS

SEQ ID NO: 256 - protein, 108 aa, synthetic construct; identical to SEQ ID NO: 248

SEQ ID NO: 257 - protein, 121 aa, synthetic construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTLVTVSS

SEQ ID NO: 258 - protein, 108 aa, synthetic construct; identical to SEQ ID NO: 248

SEQ ID NO: 259 - skipped

SEQ ID NO: 260 - protein, 17 aa, synthetic construct
  VARIANT 1: note = G or H
  VARIANT 2: note = I or F
  VARIANT 3: note = I, N, or M
  VARIANT 5: note = G, N, H, S, R, I, or A
  VARIANT 6: note = A, N, H, S, T, F, or Y
  VARIANT 7: note = A, D, or G
  VARIANT 8: note = T, E, K, V, Q, or A
  VARIANT 9: note = A or T
  VARIANT 10: note = N or K
  VARIANT 12: note = A or N
  VARIANT 16: note = Q or T
XXXPXXXXXXYXQKFXG

SEQ ID NO: 261 - skipped

SEQ ID NO: 262 - protein, 11 aa, synthetic construct
  VARIANT 1: note = R or G
  VARIANT 2: note = A or T
  VARIANT 4: note = Q or E
  VARIANT 5: note = E, N, T, S, A, K, D, G, R, or Q
  VARIANT 7: note = Y or S
  VARIANT 9: note = A or V
XXSXXIXGXLN

SEQ ID NO: 263 - skipped

SEQ ID NO: 264 - protein, 9 aa, synthetic construct
  VARIANT 2: note = S, N, D, Q, A, or E
  VARIANT 3: note = T, I, or S
  VARIANT 4: note = Y, L, or F
  VARIANT 5: note = D, G, T, E, Q, A, or Y
  VARIANT 6: note = A, T, R, S, K, or Y
QXXXXXPWT

SEQ ID NO: 265-268 - skipped (4 entries)

SEQ ID NO: 269 - protein, 17 aa, synthetic construct
  VARIANT 3: note = T, N, S, Q, E, H, R, or A
  VARIANT 4: note = Y, W, F, or H
  VARIANT 5: note = A, S, Q, E, or T
  VARIANT 7: note = G or E
  VARIANT 8: note = S or I
  VARIANT 10: note = A or G
DIXXXGXXTXYADSVKG

SEQ ID NO: 270 - protein, 12 aa, synthetic construct
  VARIANT 1: note = S or A
  VARIANT 2: note = N, H, A, D, L, Q, Y, or R
  VARIANT 3: note = A, N, S, or G
  VARIANT 5: note = A, V, R, E, or S
  VARIANT 7: note = D or S
  VARIANT 8: note = D, N, Q, E, S, T, or L
  VARIANT 10: note = L, F, or M
  VARIANT 12: note = I, Y, or V
XXXYXWXXAXDX

SEQ ID NO: 271 - protein, 7 aa, synthetic construct
  VARIANT 5: note = S, D, E, Q, S, or A
  VARIANT 6: note = S, D, E, A, or Q
GFTFXXY

SEQ ID NO: 272 - skipped

SEQ ID NO: 273 - protein, 12 aa, synthetic construct
  VARIANT 1: note = S or A
  VARIANT 2: note = N, H, A, D, L, Q, Y, or R
  VARIANT 3: note = A, N, S, or G
  VARIANT 5: note = A, V, R, E, or S
  VARIANT 7: note = D or S
  VARIANT 8: note = D, N, Q, E, S, T, or L
  VARIANT 10: note = L, F, or M
  VARIANT 12: note = I, Y, or V
XXXYXWXXAXDX

SEQ ID NO: 274 - protein, 30 aa, synthetic construct
QVQLVQSGAEVKKPGSSVKVSCKASGGTFS

SEQ ID NO: 275 - protein, 14 aa, synthetic construct
WVRQAPGQGLEWMG

SEQ ID NO: 276 - protein, 32 aa, synthetic construct
RVTITADESTSTAYMELSSLRSEDTAVYYCAR

SEQ ID NO: 277 - protein, 11 aa, synthetic construct
WGQGTLVTVSS

SEQ ID NO: 278 - protein, 25 aa, synthetic construct [sequence omitted]

SEQ ID NO: 279 - protein, 19 aa, synthetic construct
AISWVRQAPGQGLEWMGGI

SEQ ID NO: 280 - protein, 41 aa, synthetic construct
ANYAQKFQGRVTITADESTSTAYMELSSLRSEDTAVYYCAR

SEQ ID NO: 281 - protein, 30 aa, synthetic construct
EVQLVESGGGLVQPGGSLRLSCAASGFTFS

SEQ ID NO: 282 - protein, 14 aa, synthetic construct
WVRQAPGKGLEWVS

SEQ ID NO: 283 - protein, 32 aa, synthetic construct
RFTISRDNAKNSLYLQMNSLRAEDTAVYYCAR

SEQ ID NO: 284 - protein, 11 aa, synthetic construct
WGQGTMVTVSS

SEQ ID NO: 285 - protein, 11 aa, synthetic construct
WGQGTLVTVSS

SEQ ID NO: 286 - protein, 25 aa, synthetic construct [sequence omitted]

SEQ ID NO: 287 - protein, 19 aa, synthetic construct
AMSWVRQAPGKGLEWVSDI

SEQ ID NO: 288 - protein, 41 aa, synthetic construct
TAYADSVKGRFTISRDNAKNSLYLQMNSLRAEDTAVYYCAR

SEQ ID NO: 289 - protein, 23 aa, synthetic construct
DIQMTQSPSSLSASVGDRVTITC

SEQ ID NO: 290 - protein, 15 aa, synthetic construct
WYQQKPGKAPKLLIY

SEQ ID NO: 291 - protein, 32 aa, synthetic construct
GVPSRFSGSGSGTDFTLTISSLQPEDFATYYC

SEQ ID NO: 292 - protein, 10 aa, synthetic construct
FGGGTKVEIK

SEQ ID NO: 293 - protein, 23 aa, synthetic construct
EIVLTQSPGTLSLSPGERATLSC

SEQ ID NO: 294 - protein, 15 aa, synthetic construct
WYQQKPGQAPRLLIY

SEQ ID NO: 295 - protein, 32 aa, synthetic construct
GIPDRFSGSGSGTDFTLTISRLEPEDFAVYYC

SEQ ID NO: 296 - protein, 10 aa, synthetic construct
FGQGTKVEIK

SEQ ID NO: 297 - protein, 133 aa, synthetic construct; identical to SEQ ID NO: 126

SEQ ID NO: 298 - protein, 214 aa, synthetic construct
DIQMTQSPSSLSASVGDRVTITCRASQEIYGALNWYQQKPGKAPKLLIYGATNLQSGVPSRFSGSGSGTDFTLTISSLQPEDFATYYCQSTYDAPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 299 - protein, 597 aa, synthetic construct
QVQLVQSGAEVKKPGSSVKVSCKASGGTFSSYAISWVRQAPGQGLEWMGGIIPGAATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 300 - protein, 448 aa, synthetic construct
QVQLVQSGAEVKKPGSSVKVSCKASGGTFSSYAISWVRQAPGQGLEWMGGIIPGAATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 301 - protein, 449 aa, synthetic construct
QVQLVQSGAEVKKPGSSVKVSCKASGGTFSSYAISWVRQAPGQGLEWMGGIIPGAATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 302 - protein, 215 aa, synthetic construct
EIVLTQSPGTLSLSPGERATLSCRASQSVSSNLAWYQQKPGQAPRLLIYGASSRATGIPDRFSGSGSGTDFTLTISRLEPEDFAVYYCQQYGSSPPVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 303 - protein, 599 aa, synthetic construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 304 - protein, 450 aa, synthetic construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 305 - protein, 451 aa, synthetic construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 306 - protein, 215 aa, synthetic construct; identical to SEQ ID NO: 302

SEQ ID NO: 307 - protein, 599 aa, synthetic construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 308 - protein, 450 aa, synthetic construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 309 - protein, 451 aa, synthetic construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 310 - protein, 214 aa, synthetic construct
DIQMTQSPSSLSASVGDRVTITCRASQSIYGALNWYQQKPGKAPKLLIYGASNLQSGVPSRFSGSGSGTDFTLTISSLQPEDFATYYCQSTYTAPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 311 - protein, 597 aa, synthetic construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 312 - protein, 448 aa, synthetic construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 313 - protein, 449 aa, synthetic construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 314 - protein, 214 aa, synthetic construct; identical to SEQ ID NO: 298

SEQ ID NO: 315 - protein, 597 aa, synthetic construct; identical to SEQ ID NO: 311

SEQ ID NO: 316 - protein, 448 aa, synthetic construct; identical to SEQ ID NO: 312

SEQ ID NO: 317 - protein, 449 aa, synthetic construct; identical to SEQ ID NO: 313

SEQ ID NO: 318 - protein, 215 aa, synthetic construct; identical to SEQ ID NO: 302

SEQ ID NO: 319 - protein, 599 aa, synthetic construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 320 - protein, 450 aa, synthetic construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 321 - protein, 451 aa, synthetic construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 322 - protein, 215 aa, synthetic construct; identical to SEQ ID NO: 302

SEQ ID NO: 323 - protein, 599 aa, synthetic construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 324 - protein, 450 aa, synthetic construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 325 - protein, 451 aa, synthetic construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 326 - protein, 214 aa, synthetic construct; identical to SEQ ID NO: 298

SEQ ID NO: 327 - protein, 597 aa, synthetic construct
QVQLVQSGAEVKKPGSSVKVSCKASGGTFSSYAISWVRQAPGQGLEWMGGIIPGAATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEELLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 328 - protein, 448 aa, synthetic construct; identical to SEQ ID NO: 300

SEQ ID NO: 329 - protein, 449 aa, synthetic construct; identical to SEQ ID NO: 301

SEQ ID NO: 330 - protein, 215 aa, synthetic construct; identical to SEQ ID NO: 302

SEQ ID NO: 331 - protein, 599 aa, synthetic construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEELLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 332 - protein, 450 aa, synthetic construct; identical to SEQ ID NO: 304

SEQ ID NO: 333 - protein, 451 aa, synthetic construct; identical to SEQ ID NO: 305

SEQ ID NO: 334 - protein, 215 aa, synthetic construct; identical to SEQ ID NO: 302

SEQ ID NO: 335 - protein, 599 aa, synthetic construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEELLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 336 - protein, 450 aa, synthetic construct; identical to SEQ ID NO: 308

SEQ ID NO: 337 - protein, 451 aa, synthetic construct; identical to SEQ ID NO: 309

SEQ ID NO: 338 - protein, 214 aa, synthetic construct; identical to SEQ ID NO: 310

SEQ ID NO: 339 - protein, 597 aa, synthetic construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEELLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 340 - protein, 448 aa, synthetic construct; identical to SEQ ID NO: 312

SEQ ID NO: 341 - protein, 449 aa, synthetic construct; identical to SEQ ID NO: 313

SEQ ID NO: 342 - protein, 214 aa, synthetic construct; identical to SEQ ID NO: 298

SEQ ID NO: 343 - protein, 597 aa, synthetic construct; identical to SEQ ID NO: 339

SEQ ID NO: 344 - protein, 448 aa, synthetic construct; identical to SEQ ID NO: 312

SEQ ID NO: 345 - protein, 449 aa, synthetic construct; identical to SEQ ID NO: 313

SEQ ID NO: 346 - protein, 215 aa, synthetic construct; identical to SEQ ID NO: 302

SEQ ID NO: 347 - protein, 599 aa, synthetic construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEELLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 348 - protein, 450 aa, synthetic construct; identical to SEQ ID NO: 320

SEQ ID NO: 349 - protein, 451 aa, synthetic construct; identical to SEQ ID NO: 321

SEQ ID NO: 350 - protein, 215 aa, synthetic construct; identical to SEQ ID NO: 302

SEQ ID NO: 351 - protein, 599 aa, synthetic construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEELLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 352 - protein, 450 aa, synthetic construct; identical to SEQ ID NO: 324

SEQ ID NO: 353 - protein, 451 aa, synthetic construct; identical to SEQ ID NO: 325

SEQ ID NO: 354 - protein, 133 aa, synthetic construct
APTSSSTKKTQLQLEHLLLDLQMILNGINNYKNPKLTEMLTAKFYMPKKATELKHLQCLEEELKPLEEVLNLAQSKNFHLRPRDLISDINVIVLELKGSETTFMCEYADETATIVEFLNRWITFAQSIISTLT

SEQ ID NO: 355 - protein, 133 aa, synthetic construct
APTSSSTKKTQLQLEHLLLDLQMILNGINNYKNPKLTEMLTAKFYMPKKATELKHLQCLEEELKPLEEVLNLAQSKNFHLRPRDLISTINVIVLELKGSETTFMCEYADETATIVEFLNRWITFAQSIISTLT

SEQ ID NO: 356 - protein, 133 aa, synthetic construct
APTSSSTKKTQLQLEHLLLDLQMILNGINNYKNPKLTEMLTAKFYMPKKATELKHLQCLEEELKPLEEVLNLAQSKNFHLRPRDLISRINVIVLELKGSETTFMCEYADETATIVEFLNRWITFAQSIISTLT

SEQ ID NO: 357 - protein, 133 aa, synthetic construct
APTSSSTKKTQLQLEHLLLDLQMILNGINNYKNPKLTEMLTAKFYMPKKATELKHLQCLEEELKPLEEVLNLAQSKNFHLRPRDLISRINVIVLELKGSETTFMCEYADETATIVEFLNRWITFCQSIISTLT

SEQ ID NO: 358 - protein, 133 aa, synthetic construct
APTSSSTKKTQLQLEHLLLDLQMILNGINNYKNPKLTEMLTAKFYMPKKATELKHLQCLEEELKPLEEVLNLAQSKNFHLRPRDLISTINVIVLELKGSETTFMCEYADETATIVEFLNRWITFCQSIISTLT

SEQ ID NO: 359 - protein, 133 aa, synthetic construct
APTSSSTKKTQLQLEHLLLDLQMILNGINNYKNPKLTEMLTAKFYMPKKATELKHLQCLEEELKPLEEVLNLAQSKNFHLRPRDLISDINVIVLELKGSETTFMCEYADETATIVEFLNRWITFCQSIISTLT

SEQ ID NO: 360 - protein, 133 aa, synthetic construct
APTSSSTKKTQLQLEHLLLDLQMILNGINNYKNPKLTDMLTAKFYMPKKATELKHLQCLEEELKPLEEVLNLAQSKNFHLRPRDLISRINVIVLELKGSETTFMCEYADETATIVEFLNRWITFCQSIISTLT

SEQ ID NO: 361 - protein, 133 aa, synthetic construct
APTSSSTKKTQLQLEHLLLDLQMILNGINNYKNPKLTDMLTAKFYMPKKATELKHLQCLEEELKPLEEVLNLAQSKNFHLRPRDLISTINVIVLELKGSETTFMCEYADETATIVEFLNRWITFCQSIISTLT

SEQ ID NO: 362 - protein, 133 aa, synthetic construct
APTSSSTKKTQLQLEHLLLDLQMILNGINNYKNPKLTDMLTAKFYMPKKATELKHLQCLEEELKPLEEVLNLAQSKNFHLRPRDLISDINVIVLELKGSETTFMCEYADETATIVEFLNRWITFCQSIISTLT

SEQ ID NO: 363 - protein, 133 aa, synthetic construct
APTSSSTKKTQLQLEHLLLDLQMILNGINNYKNPKLTAMLTKKFYMPKKATELKHLQCLEEELKPLEEVLNLAQSKNFHLRPRDLISRINVIVLELKGSETTFMCEYADETATIVEFLNRWITFCQSIISTLT

SEQ ID NO: 364 - protein, 133 aa, synthetic construct
APTSSSTKKTQLQLEHLLLDLQMILNGINNYKNPKLTAMLTKKFYMPKKATELKHLQCLEEELKPLEEVLNLAQSKNFHLRPRDLISTINVIVLELKGSETTFMCEYADETATIVEFLNRWITFCQSIISTLT

SEQ ID NO: 365 - protein, 133 aa, synthetic construct
APTSSSTKKTQLQLEHLLLDLQMILNGINNYKNPKLTAMLTKKFYMPKKATELKHLQCLEEELKPLEEVLNLAQSKNFHLRPRDLISDINVIVLELKGSETTFMCEYADETATIVEFLNRWITFCQSIISTLT

SEQ ID NO: 366 - protein, 133 aa, synthetic construct
APTSSSTKKTQLQLEHLLLDLQMILNGINNYKNPKLTRMLTAKFYMPKKATELKHLQCLEEQLKPLEEVLNLAQSKNFHLRPRDLISRINVIVLELKGSETTFMCEYADETATIVEFLNRWITFCQSIISTLT

SEQ ID NO: 367 - protein, 133 aa, synthetic construct
APTSSSTKKTQLQLEHLLLDLQMILNGINNYKNPKLTRMLTAKFYMPKKATELKHLQCLEEQLKPLEEVLNLAQSKNFHLRPRDLISTINVIVLELKGSETTFMCEYADETATIVEFLNRWITFCQSIISTLT

SEQ ID NO: 368 - protein, 133 aa, synthetic construct
APTSSSTKKTQLQLEHLLLDLQMILNGINNYKNPKLTRMLTAKFYMPKKATELKHLQCLEEQLKPLEEVLNLAQSKNFHLRPRDLISDINVIVLELKGSETTFMCEYADETATIVEFLNRWITFCQSIISTLT

SEQ ID NO: 369 - protein, 133 aa, synthetic construct; identical to SEQ ID NO: 356

SEQ ID NO: 370 - protein, 133 aa, synthetic construct; identical to SEQ ID NO: 355

SEQ ID NO: 371 - protein, 133 aa, synthetic construct; identical to SEQ ID NO: 354

SEQ ID NO: 372 - protein, 133 aa, synthetic construct
APTSSSTKKTQLQLEHLLLDLQMILNGINNYKNPKLTDMLTAKFYMPKKATELKHLQCLEEELKPLEEVLNLAQSKNFHLRPRDLISRINVIVLELKGSETTFMCEYADETATIVEFLNRWITFAQSIISTLT

SEQ ID NO: 373 - protein, 133 aa, synthetic construct
APTSSSTKKTQLQLEHLLLDLQMILNGINNYKNPKLTDMLTAKFYMPKKATELKHLQCLEEELKPLEEVLNLAQSKNFHLRPRDLISTINVIVLELKGSETTFMCEYADETATIVEFLNRWITFAQSIISTLT

SEQ ID NO: 374 - protein, 133 aa, synthetic construct
APTSSSTKKTQLQLEHLLLDLQMILNGINNYKNPKLTDMLTAKFYMPKKATELKHLQCLEEELKPLEEVLNLAQSKNFHLRPRDLISDINVIVLELKGSETTFMCEYADETATIVEFLNRWITFAQSIISTLT

SEQ ID NO: 375 - protein, 133 aa, synthetic construct
APTSSSTKKTQLQLEHLLLDLQMILNGINNYKNPKLTAMLTKKFYMPKKATELKHLQCLEEELKPLEEVLNLAQSKNFHLRPRDLISRINVIVLELKGSETTFMCEYADETATIVEFLNRWITFAQSIISTLT

SEQ ID NO: 376 - protein, 133 aa, synthetic construct
APTSSSTKKTQLQLEHLLLDLQMILNGINNYKNPKLTAMLTKKFYMPKKATELKHLQCLEEELKPLEEVLNLAQSKNFHLRPRDLISTINVIVLELKGSETTFMCEYADETATIVEFLNRWITFAQSIISTLT

SEQ ID NO: 377 - protein, 133 aa, synthetic construct
APTSSSTKKTQLQLEHLLLDLQMILNGINNYKNPKLTAMLTKKFYMPKKATELKHLQCLEEELKPLEEVLNLAQSKNFHLRPRDLISDINVIVLELKGSETTFMCEYADETATIVEFLNRWITFAQSIISTLT

SEQ ID NO: 378 - protein, 133 aa, synthetic construct
APTSSSTKKTQLQLEHLLLDLQMILNGINNYKNPKLTRMLTAKFYMPKKATELKHLQCLEEQLKPLEEVLNLAQSKNFHLRPRDLISRINVIVLELKGSETTFMCEYADETATIVEFLNRWITFAQSIISTLT

SEQ ID NO: 379 - protein, 133 aa, synthetic construct
APTSSSTKKTQLQLEHLLLDLQMILNGINNYKNPKLTRMLTAKFYMPKKATELKHLQCLEEQLKPLEEVLNLAQSKNFHLRPRDLISTINVIVLELKGSETTFMCEYADETATIVEFLNRWITFAQSIISTLT

SEQ ID NO: 380 - protein, 133 aa, synthetic construct
APTSSSTKKTQLQLEHLLLDLQMILNGINNYKNPKLTRMLTAKFYMPKKATELKHLQCLEEQLKPLEEVLNLAQSKNFHLRPRDLISDINVIVLELKGSETTFMCEYADETATIVEFLNRWITFAQSIISTLT

SEQ ID NO: 381 - protein, 133 aa, synthetic construct
APTSSSTKKTQLQLEHLLLDLQMILNGINNYKNPKLTRMLTAKFYMPKKATELKHLQCLEEELKPLEEVLNLAQSKNFHLRPRDLISRINVIVLELKGSETTFMCEYADETATIVEFLNRWITFAQSIISTLT

SEQ ID NO: 382 - protein, 133 aa, synthetic construct
APTSSSTKKTQLQLEHLLLDLQMILNGINNYKNPKLTRMLTAKFYMPKKATELKHLQCLEEELKPLEEVLNLAQSKNFHLRPRDLISTINVIVLELKGSETTFMCEYADETATIVEFLNRWITFAQSIISTLT

SEQ ID NO: 383 - protein, 133 aa, synthetic construct
APTSSSTKKTQLQLEHLLLDLQMILNGINNYKNPKLTRMLTAKFYMPKKATELKHLQCLEEELKPLEEVLNLAQSKNFHLRPRDLISDINVIVLELKGSETTFMCEYADETATIVEFLNRWITFAQSIISTLT

SEQ ID NO: 384-385 - skipped (2 entries)

SEQ ID NO: 386 - protein, 52 aa, synthetic construct
  VARIANT 6..49: note = GGGS repeats may be deleted
  VARIANT 51..52: note = Residue may be deleted
SGGGSGGGSGGGSGGGSGGGSGGGSGGGSGGGSGGGSGGGSGGGSGGGSGGG

SEQ ID NO: 387 - protein, 64 aa, synthetic construct
  VARIANT 7..61: note = GGGGS repeats may be deleted
  VARIANT 63..64: note = Residue may be deleted
SGGGGSGGGGSGGGGSGGGGSGGGGSGGGGSGGGGSGGGGSGGGGSGGGGSGGGGSGGGGSGGG

SEQ ID NO: 388 - protein, 76 aa, synthetic construct
  VARIANT 8..73: note = GGGGGS repeats may be deleted
  VARIANT 75..76: note = Residue may be deleted
SGGGGGSGGGGGSGGGGGSGGGGGSGGGGGSGGGGGSGGGGGSGGGGGSGGGGGSGGGGGSGGGGGSGGGGGSGGG

SEQ ID NO: 389 - protein, 16 aa, synthetic construct
SGGGGSGGGGSGGGGS

SEQ ID NO: 390 - protein, 133 aa, synthetic construct [sequence omitted]

SEQ ID NO: 391 - protein, 133 aa, synthetic construct
QGQDRHMIRMRQLIDIVDQLKNYVNDLVPEFLPAPEDVETNCEWSAFSCFQKAQLKSANTGNNERIINVSIKKLKRKPPSTNAGGGQGHELTCPSCDSYEKKPPKEFLERFKSLLQKMIHQHLSSRTHGSEDS

SEQ ID NO: 392 - protein, 133 aa, synthetic construct
QGQDRHMIRMRQLIDIVDQLKNYVNDLVPEFLPAPEDVETNCEWSAFSCFQKAQLKSANTGNNERIINVSIKKLKRKPPSTNAGGGQGHALTCPSCDSYEKKPPKEFLERFKSLLQKMIHQHLSSRTHGSEDS

SEQ ID NO: 393 - protein, 133 aa, synthetic construct
QGQDRHMIRMRQLIDIVDQLKNYVNDLVPEFLPAPEDVETNCEWSAFSCFQKAQLASANTGNNERIINVSIKKLARKPPSTNAGGGQGHALTCPSCDSYEKKPPKEFLERFKSLLQKMIHQHLSSRTHGSEDS

SEQ ID NO: 394 - protein, 133 aa, synthetic construct
QGQDRHMIRMRQLIDIVDQLKNYVNDLVPEFLPAPEDVETNCEWSAFSCFQKAQLESANTGNNERIINVSIKKLARKPPSTNAGGGQGHALTCPSCDSYEKKPPKEFLERFKSLLQKMIHQHLSSRTHGSEDS

SEQ ID NO: 395 - protein, 133 aa, synthetic construct
QGQDRHMIRMRQLIDIVDQLKNYVNDLVPEFLPAPEDVETNCEWSAFSCFQKAQLASANTGNNERIINVSIKKLERKPPSTNAGGGQGHALTCPSCDSYEKKPPKEFLERFKSLLQKMIHQHLSSRTHGSEDS

SEQ ID NO: 396 - protein, 133 aa, synthetic construct
QGQDRHMIRMRQLIDIVDQLKNYVNDLVPEFLPAPEDVETNCEWSAFSCFQKAQLASANTGNNERIINVSIKKLARKPPSTNAGGGQGHELTCPSCDSYEKKPPKEFLERFKSLLQKMIHQHLSSRTHGSEDS

SEQ ID NO: 397 - protein, 133 aa, synthetic construct
QGQDRHMIRMRQLIDIVDQLKNYVNDLVPEFLPAPEDVETNCEWSAFSCFQKAQLKSANTGNNERIINVSIKKLKRKPPGGGSGEGSGGSLTCPSCDSYEKKPPKEFLERFKSLLQKMIHQHLSSRTHGSEDS

SEQ ID NO: 398 - protein, 133 aa, synthetic construct
QGQDRHMIRMRQLIDIVDQLKNYVNDLVPEFLPAPEDVETNCEWSAFSCFQKAQLKSANTGNNERIINVSIKKLKRKPPGGGSGGGSGGELTCPSCDSYEKKPPKEFLERFKSLLQKMIHQHLSSRTHGSEDS

SEQ ID NO: 399 - protein, 133 aa, synthetic construct
QGQDRHMIRMRQLIDIVDQLKNYVNDLVPEFLPAPEDVETNCEWSAFSCFQKAQLKSANTGNNERIINVSIKKLKRKPPSTNAGGGSGGELTCPSCDSYEKKPPKEFLERFKSLLQKMIHQHLSSRTHGSEDS

SEQ ID NO: 400 - protein, 133 aa, synthetic construct
QGQDRHMIRMRQLIDIVDQLKNYVNDLVPEFLPAPEDVETNCEWSAFSCFQKAQLKSANTGNNERIINVSIKKLKRKPPSTNAGGGSGGSLTCPSCDSYEKKPPKEFLERFKSLLQKMIHQHLSSRTHGSEDS

SEQ ID NO: 401 - protein, 133 aa, synthetic construct
QGQDRHMIRMRQLIDIVDQLKNYVNDLVPEFLPAPEDVETNCEWSAFSCFQKAQLKSANTGNNERIINVSIKKLKRKPPSTNAGGGGGGELTCPSCDSYEKKPPKEFLERFKSLLQKMIHQHLSSRTHGSEDS

SEQ ID NO: 402 - protein, 133 aa, synthetic construct
QGQDRHMIRMRQLIDIVDQLKNYVNDLVPEFLPAPEDVETNCEWSAFSCFQKAQLKSANTGNNERIINVSIKKLKRKPPSTNAGGGGGGGLTCPSCDSYEKKPPKEFLERFKSLLQKMIHQHLSSRTHGSEDS

SEQ ID NO: 403 - protein, 133 aa, synthetic construct
QGQDRHMIRMRQLIDIVDQLKNYVNDLVPEFLPAPEDVETNCEWSAFSCFQKAQLKSANTGNNERIINVSIKKLKRKPPSTNAGGGEGGGLTCPSCDSYEKKPPKEFLERFKSLLQKMIHQHLSSRTHGSEDS

SEQ ID NO: 404 - protein, 133 aa, synthetic construct
QGQDRHMIRMRQLIDIVDQLKNYVNDLVPEFLPAPEDVETNCEWSAFSCFQKAQLKSANTGNNERIINVSIKKLKRKPPGGGSGGGSGGGSGCPSCDSYEKKPPKEFLERFKSLLQKMIHQHLSSRTHGSEDS

SEQ ID NO: 405 - protein, 133 aa, synthetic construct
QGQDRHMIRMRQLIDIVDQLKNYVNDLVPEFLPAPEDVETNCEWSAFSCFQKAQLKSANTGNNERIINVSIKKLKRKPPGGGGGGGGGGGGGCPSCDSYEKKPPKEFLERFKSLLQKMIHQHLSSRTHGSEDS

SEQ ID NO: 406 - protein, 133 aa, synthetic construct
QGQDRHMIRMRQLIDIVDQLKNYVNDLVPEFLPAPEDVETNCEWSAFSCFQKAQLKSANTGNNERIINVSIKKLKRKPPGGGSGGGEGGGSGCPSCDSYEKKPPKEFLERFKSLLQKMIHQHLSSRTHGSEDS

SEQ ID NO: 407 - protein, 133 aa, synthetic construct
QGQDRHMIRMRQLIDIVDQLKNYVNDLVPEFLPAPEDVETNCEWSAFSCFQKAQLKSANTGNNERIINVSIKKLKRKPPGGGEGGGEGGGSGCPSCDSYEKKPPKEFLERFKSLLQKMIHQHLSSRTHGSEDS

SEQ ID NO: 408 - protein, 133 aa, synthetic construct
QGQDRHMIRMRQLIDIVDQLKNYVNDLVPEFLPAPEDVETNCEWSAFSCFQKAQLKSANTGNNERIINVSIKKLKRKPPSTGGGSGGGSGGSCPSCDSYEKKPPKEFLERFKSLLQKMIHQHLSSRTHGSEDS

SEQ ID NO: 409 - protein, 133 aa, synthetic construct
QGQDRHMIRMRQLIDIVDQLKNYVNDLVPEFLPAPEDVETNCEWSAFSCFQKAQLKSANTGNNERIINVSIKKLKRKPPSTGGGEGGGSGGSCPSCDSYEKKPPKEFLERFKSLLQKMIHQHLSSRTHGSEDS

SEQ ID NO: 410 - protein, 133 aa, synthetic construct
QGQDRHMIRMRQLIDIVDQLKNYVNDLVPEFLPAPEDVETNCEWSAFSCFQKAQLKSANTGNNERIINVSIKKLKRKPPGGGGGEGGGGGGGCPSCDSYEKKPPKEFLERFKSLLQKMIHQHLSSRTHGSEDS

SEQ ID NO: 411 - protein, 133 aa, synthetic construct
QGQDRHMIRMRQLIDIVDQLKNYVNDLVPEFLPAPEDVETNCEWSAFSCFQKAQLKSANTGNNERIINVSIKKLKRKPPGGGSGGGSGGEGGCPSCDSYEKKPPKEFLERFKSLLQKMIHQHLSSRTHGSEDS

SEQ ID NO: 412 - protein, 133 aa, synthetic construct
QGQDRHMIRMRQLIDIVDQLKNYVNDLVPEFLPAPEDVETNCEWSAFSCFQKAQLKSANTGNNERIINVSIKKLKRKPPGGGGGGGGGGEGGCPSCDSYEKKPPKEFLERFKSLLQKMIHQHLSSRTHGSEDS

SEQ ID NO: 413 - protein, 133 aa, synthetic construct
QGQDRHMIRMRQLIDIVDQLKNYVNDLVPEFLPAPEDVETNCEWSAFSCFQKAQLKSANTGNNERIINVSIKKLKRKPPSTGGGSGGGSEGSCPSCDSYEKKPPKEFLERFKSLLQKMIHQHLSSRTHGSEDS

SEQ ID NO: 414 - protein, 133 aa, synthetic construct
QGQDRHMIRMRQLIDIVDQLKNYVNDLVPEFLPAPEDVETNCEWSAFSCFQKAQLKSANTGNNERIINVSIKKLKRKPPSTGGGGGGGGEGGCPSCDSYEKKPPKEFLERFKSLLQKMIHQHLSSRTHGSEDS

SEQ ID NO: 415 - protein, 133 aa, synthetic construct
QGQDRHMIRMRQLIDIVDQLKNYVNDLVPEFLPAPEDVETNCEWSAFSCFQKAQLKSANTGNNERIINVSIKKLKRKPPSTNAGGGGGGEGTCPSCDSYEKKPPKEFLERFKSLLQKMIHQHLSSRTHGSEDS

SEQ ID NO: 416 - protein, 133 aa, synthetic construct
QGQDRHMIRMRQLIDIVDQLKNYVNDLVPEFLPAPEDVETNCEWSAFSCFQKAQLKSANTGNNERIINVSIKKLKRKPPSTGGGGGGGGEGTCPSCDSYEKKPPKEFLERFKSLLQKMIHQHLSSRTHGSEDS

SEQ ID NO: 417 - protein, 133 aa, synthetic construct
QGQDRHMIRMRQLIDIVDQLKNYVNDLVPEFLPAPEDVETNCEWSAFSCFQKAQLKSANTGNNERIINVSIKKLKRKPPSTGGGGEGGGEGTCPSCDSYEKKPPKEFLERFKSLLQKMIHQHLSSRTHGSEDS

SEQ ID NO: 418 - protein, 133 aa, synthetic construct
QGQDRHMIRMRQLIDIVDQLKNYVNDLVPEFLPAPEDVETNCEWSAFSCFQKAQLKSANTGNNERIINVSIKKLKRKPPSTGGGEGGGGEGTCPSCDSYEKKPPKEFLERFKSLLQKMIHQHLSSRTHGSEDS

SEQ ID NO: 419 - protein, 133 aa, synthetic construct
QGQDRHMIRMRQLIDIVDQLKNYVNDLVPEFLPAPEDVETNCEWSAFSCFQKAQLKSANTGNNERIINVSIKKLKRKPPGGGGEGGGGGEGGCPSCDSYEKKPPKEFLERFKSLLQKMIHQHLSSRTHGSEDS

SEQ ID NO: 420 - protein, 133 aa, synthetic construct
QGQDRHMIRMRQLIDIVDQLKNYVNDLVPEFLPAPEDVETNCEWSAFSCFQKAQLKSANTGNNERIINVSIKKLKRKPPGGGEGGGGGGEGGCPSCDSYEKKPPKEFLERFKSLLQKMIHQHLSSRTHGSEDS

SEQ ID NO: 421 - protein, 133 aa, synthetic construct
QGQDRHMIRMRQLIDIVDQLKNYVNDLVPEFLPAPEDVETNCEWSAFSCFQKAQLKSANTGNNERIINVSIKKLKRKPPGGEGGGGGGGEGGCPSCDSYEKKPPKEFLERFKSLLQKMIHQHLSSRTHGSEDS

SEQ ID NO: 422 - protein, 133 aa, synthetic construct
QGQDRHMIRMDQLIDIVDQLKNYVNDLVPEFLPAPEDVETNCEWSAFSCFQKAQLKSANTGNNERIINVSIKKLKRKPPSTNAGGGQGHELTCPSCDSYEKKPPKEFLERFKSLLQKMIHQHLSSRTHGSEDS

SEQ ID NO: 423 - protein, 133 aa, synthetic construct
QGQDRHMIRMEQLIDIVDQLKNYVNDLVPEFLPAPEDVETNCEWSAFSCFQKAQLKSANTGNNERIINVSIKKLKRKPPSTNAGGGQGHELTCPSCDSYEKKPPKEFLERFKSLLQKMIHQHLSSRTHGSEDS

SEQ ID NO: 424 - protein, 133 aa, synthetic construct
QGQDRHMIRMRQLDDIVAQLKNYVNDLVPEFLPAPEDVETNCEWSAFSCFQKAQLKSANTGNNERIINVSIKKLKRKPPSTNAGGGQGHELTCPSCDSYEKKPPKEFLERFKSLLQAMIHQHLSSRTHGSEDS

SEQ ID NO: 425 - protein, 133 aa, synthetic construct
QGQDRHMIRMRQLIDIVDQLKNYVNDLVPEFLPAPEDVETNCEWSAFSCFQKAQLKSANTGNNERIINVSIKKLKEKPPSTNAGGGQGHELTCPSCDSYEKKPPKEFLERFKSLLQKMIHQHLSSRTHGSEDS

SEQ ID NO: 426 - protein, 133 aa, synthetic construct
QGQDFHMIRMRQLIDIVDQLKNYVNDLVPEFLPAPEDVETNCEWSAFSCFQKAQLKSANTGNNERIINVSIKKLKRKPPSTNAGGGQGHELTCPSCDSYEKKPPKEFLERFKSLLQKMIHQHLSSRTHGSEDS

SEQ ID NO: 427 - protein, 133 aa, synthetic construct
QGQDRHMIRMRQLIDIVDQLKNYVNDLVPEFLPAPEDVETNCEWSAFSCFQKAQLKSANTGNNERIINVSIKKLKFKPPSTNAGGGQGHELTCPSCDSYEKKPPKEFLERFKSLLQKMIHQHLSSRTHGSEDS

SEQ ID NO: 428 - protein, 133 aa, synthetic construct
QGQDRHMERMRQLIDIVDQLKNYVNDLVPEFLPAPEDVETNCEWSAFSCFQKAQLKSANTGNNERIINVSIKKLKRKPPSTNAGGGQGHELTCPSCDSYEKKPPKEFLERFKSLLQKMIHQHLSSRTHGSEDS

SEQ ID NO: 429 - protein, 133 aa, synthetic construct
QGQDAHMIRMRQLIDIVDQLKNYVNDLVPEFLPAPEDVETNCEWSAFSCFQKAQLKSANTGNNERIINVSIKKLKRKPPSTNAGGGQGHELTCPSCDSYEKKPPKEFLERFKSLLQKMIHQHLSSRTHGSEDS

SEQ ID NO: 430 - protein, 133 aa, synthetic construct
QGQDEHMIRMRQLIDIVDQLKNYVNDLVPEFLPAPEDVETNCEWSAFSCFQKAQLKSANTGNNERIINVSIKKLKRKPPSTNAGGGQGHELTCPSCDSYEKKPPKEFLERFKSLLQKMIHQHLSSRTHGSEDS

SEQ ID NO: 431 - protein, 133 aa, synthetic construct
QGQDSHMIRMRQLIDIVDQLKNYVNDLVPEFLPAPEDVETNCEWSAFSCFQKAQLKSANTGNNERIINVSIKKLKRKPPSTNAGGGQGHELTCPSCDSYEKKPPKEFLERFKSLLQKMIHQHLSSRTHGSEDS

SEQ ID NO: 432 - protein, 133 aa, synthetic construct
QGQDTHMIRMRQLIDIVDQLKNYVNDLVPEFLPAPEDVETNCEWSAFSCFQKAQLKSANTGNNERIINVSIKKLKRKPPSTNAGGGQGHELTCPSCDSYEKKPPKEFLERFKSLLQKMIHQHLSSRTHGSEDS

SEQ ID NO: 433 - protein, 133 aa, synthetic construct
QGQDNHMIRMRQLIDIVDQLKNYVNDLVPEFLPAPEDVETNCEWSAFSCFQKAQLKSANTGNNERIINVSIKKLKRKPPSTNAGGGQGHELTCPSCDSYEKKPPKEFLERFKSLLQKMIHQHLSSRTHGSEDS

SEQ ID NO: 434 - protein, 133 aa, synthetic construct
QGQDQHMIRMRQLIDIVDQLKNYVNDLVPEFLPAPEDVETNCEWSAFSCFQKAQLKSANTGNNERIINVSIKKLKRKPPSTNAGGGQGHELTCPSCDSYEKKPPKEFLERFKSLLQKMIHQHLSSRTHGSEDS

SEQ ID NO: 435 - protein, 133 aa, synthetic construct
QGQDVHMIRMRQLIDIVDQLKNYVNDLVPEFLPAPEDVETNCEWSAFSCFQKAQLKSANTGNNERIINVSIKKLKRKPPSTNAGGGQGHELTCPSCDSYEKKPPKEFLERFKSLLQKMIHQHLSSRTHGSEDS

SEQ ID NO: 436 - protein, 133 aa, synthetic construct
QGQDIHMIRMRQLIDIVDQLKNYVNDLVPEFLPAPEDVETNCEWSAFSCFQKAQLKSANTGNNERIINVSIKKLKRKPPSTNAGGGQGHELTCPSCDSYEKKPPKEFLERFKSLLQKMIHQHLSSRTHGSEDS

SEQ ID NO: 437 - protein, 133 aa, synthetic construct
QGQDLHMIRMRQLIDIVDQLKNYVNDLVPEFLPAPEDVETNCEWSAFSCFQKAQLKSANTGNNERIINVSIKKLKRKPPSTNAGGGQGHELTCPSCDSYEKKPPKEFLERFKSLLQKMIHQHLSSRTHGSEDS

SEQ ID NO: 438 - protein, 133 aa, synthetic construct
QGQDYHMIRMRQLIDIVDQLKNYVNDLVPEFLPAPEDVETNCEWSAFSCFQKAQLKSANTGNNERIINVSIKKLKRKPPSTNAGGGQGHELTCPSCDSYEKKPPKEFLERFKSLLQKMIHQHLSSRTHGSEDS

SEQ ID NO: 439 - protein, 133 aa, synthetic construct
QGQDRHMIRMRQLIDIVDQLKNYVNDLVPEFLPAPEDVETNCEWSAFSCFQKAQLKSANTGNNERIINVSIKKLKAKPPSTNAGGGQGHELTCPSCDSYEKKPPKEFLERFKSLLQKMIHQHLSSRTHGSEDS

SEQ ID NO: 440 - protein, 133 aa, synthetic construct
QGQDRHMIRMRQLIDIVDQLKNYVNDLVPEFLPAPEDVETNCEWSAFSCFQKAQLKSANTGNNERIINVSIKKLKNKPPSTNAGGGQGHELTCPSCDSYEKKPPKEFLERFKSLLQKMIHQHLSSRTHGSEDS

SEQ ID NO: 441 - protein, 133 aa, synthetic construct
QGQDRHMIRMRQLIDIVDQLKNYVNDLVPEFLPAPEDVETNCEWSAFSCFQKAQLKSANTGNNERIINVSIKKLKDKPPSTNAGGGQGHELTCPSCDSYEKKPPKEFLERFKSLLQKMIHQHLSSRTHGSEDS

SEQ ID NO: 442 - protein, 133 aa, synthetic construct
QGQDRHMIRMRQLIDIVDQLKNYVNDLVPEFLPAPEDVETNCEWSAFSCFQKAQLKSANTGNNERIINVSIKKLKSKPPSTNAGGGQGHELTCPSCDSYEKKPPKEFLERFKSLLQKMIHQHLSSRTHGSEDS

SEQ ID NO: 443 - protein, 133 aa, synthetic construct
QGQDRHMIRMRQLIDIVDQLKNYVNDLVPEFLPAPEDVETNCEWSAFSCFQKAQLKSANTGNNERIINVSIKKLKTKPPSTNAGGGQGHELTCPSCDSYEKKPPKEFLERFKSLLQKMIHQHLSSRTHGSEDS

SEQ ID NO: 444 - protein, 133 aa, synthetic construct
QGQDRHMIRMRQLIDIVDQLKNYVNDLVPEFLPAPEDVETNCEWSAFSCFQKAQLKSANTGNNERIINVSIKKLKQKPPSTNAGGGQGHELTCPSCDSYEKKPPKEFLERFKSLLQKMIHQHLSSRTHGSEDS

SEQ ID NO: 445 - protein, 133 aa, synthetic construct
QGQDRHMIRMRQLIDIVDQLKNYVNDLVPEFLPAPEDVETNCEWSAFSCFQKAQLKSANTGNNERIINVSIKKLKVKPPSTNAGGGQGHELTCPSCDSYEKKPPKEFLERFKSLLQKMIHQHLSSRTHGSEDS

SEQ ID NO: 446 - protein, 133 aa, synthetic construct
QGQDRHMIRMRQLIDIVDQLKNYVNDLVPEFLPAPEDVETNCEWSAFSCFQKAQLKSANTGNNERIINVSIKKLKIKPPSTNAGGGQGHELTCPSCDSYEKKPPKEFLERFKSLLQKMIHQHLSSRTHGSEDS

SEQ ID NO: 447 - protein, 133 aa, synthetic construct
QGQDRHMIRMRQLIDIVDQLKNYVNDLVPEFLPAPEDVETNCEWSAFSCFQKAQLKSANTGNNERIINVSIKKLKLKPPSTNAGGGQGHELTCPSCDSYEKKPPKEFLERFKSLLQKMIHQHLSSRTHGSEDS

SEQ ID NO: 448 - protein, 133 aa, synthetic construct
QGQDRHMIRMRQLIDIVDQLKNYVNDLVPEFLPAPEDVETNCEWSAFSCFQKAQLKSANTGNNERIINVSIKKLKYKPPSTNAGGGQGHELTCPSCDSYEKKPPKEFLERFKSLLQKMIHQHLSSRTHGSEDS

SEQ ID NO: 449 - protein, 133 aa, synthetic construct
QGQDRHMIRMRQLIDIVDQLKNYVNDLVPEFLPAPEDVETNCEWSAFSCFQKAQLKSANTGNNERIINVSIKKLKRAPPSTNAGGGQGHELTCPSCDSYEKKPPKEFLERFKSLLQKMIHQHLSSRTHGSEDS

SEQ ID NO: 450 - protein, 133 aa, synthetic construct
QGQDRHMIRMRQLIDIVDQLKNYVNDLVPEFLPAPEDVETNCEWSAFSCFQKAQLKSANTGNNERIINVSIKKLKREPPSTNAGGGQGHELTCPSCDSYEKKPPKEFLERFKSLLQKMIHQHLSSRTHGSEDS

SEQ ID NO: 451 - protein, 133 aa, synthetic construct
QGQDRHMIRMRQLIDIVDQLKNYVNDLVPEFLPAPEDVETNCEWSAFSCFQKAQLKSANTGNNERIINVSIAKLKRKPPSTNAGGGQGHELTCPSCDSYEKKPPKEFLERFKSLLQKMIHQHLSSRTHGSEDS

SEQ ID NO: 452 - protein, 133 aa, synthetic construct
QGQDRHMIRMRQLIDIVDQLKNYVNDLVPEFLPAPEDVETNCEWSAFSCFQKAQLKSANTGNNERIINVSIEKLKRKPPSTNAGGGQGHELTCPSCDSYEKKPPKEFLERFKSLLQKMIHQHLSSRTHGSEDS

SEQ ID NO: 453 - protein, 133 aa, synthetic construct
QGQDRHMIRMRQLIDIVDQLKNYVNDLVPEFLPAPEDVETNCEWSAFSCFQKAQLKSANTGNNERIINVSIKKLARKPPSTNAGGGQGHELTCPSCDSYEKKPPKEFLERFKSLLQKMIHQHLSSRTHGSEDS

SEQ ID NO: 454 - protein, 133 aa, synthetic construct
QGQDRHMIRMRQLIDIVDQLKNYVNDLVPEFLPAPEDVETNCEWSAFSCFQKAQLKSANTGNNERIINVSIKKLERKPPSTNAGGGQGHELTCPSCDSYEKKPPKEFLERFKSLLQKMIHQHLSSRTHGSEDS

SEQ ID NO: 455 - protein, 133 aa, synthetic construct
QGQDRHMIRMRQLIDIVDQLKNYVNDLVPEFLPAPEDVETNCEWSAFSCFQKAQLKSANTGNNERIINVSIKALKRKPPSTNAGGGQGHELTCPSCDSYEKKPPKEFLERFKSLLQKMIHQHLSSRTHGSEDS

SEQ ID NO: 456 - protein, 133 aa, synthetic construct
QGQDRHMIRMRQLIDIVDQLKNYVNDLVPEFLPAPEDVETNCEWSAFSCFQKAQLKSANTGNNERIINVSIKELKRKPPSTNAGGGQGHELTCPSCDSYEKKPPKEFLERFKSLLQKMIHQHLSSRTHGSEDS

SEQ ID NO: 457 - protein, 133 aa, synthetic construct
QGQDFHMIRMRQLIDIVDQLKNYVNDLVPEFLPAPEDVETNCEWSAFSCFQKAQLKSANTGNNERIINVSIKKLKRAPPSTNAGGGQGHELTCPSCDSYEKKPPKEFLERFKSLLQKMIHQHLSSRTHGSEDS

SEQ ID NO: 458 - protein, 133 aa, synthetic construct
QGQDFHMIRMRQLIDIVDQLKNYVNDLVPEFLPAPEDVETNCEWSAFSCFQKAQLKSANTGNNERIINVSIKKLKREPPSTNAGGGQGHELTCPSCDSYEKKPPKEFLERFKSLLQKMIHQHLSSRTHGSEDS

SEQ ID NO: 459 - protein, 133 aa, synthetic construct
QGQDFHMIRMRQLIDIVDQLKNYVNDLVPEFLPAPEDVETNCEWSAFSCFQKAQLKSANTGNNERIINVSIAKLKRKPPSTNAGGGQGHELTCPSCDSYEKKPPKEFLERFKSLLQKMIHQHLSSRTHGSEDS

SEQ ID NO: 460 - protein, 133 aa, synthetic construct
QGQDFHMIRMRQLIDIVDQLKNYVNDLVPEFLPAPEDVETNCEWSAFSCFQKAQLKSANTGNNERIINVSIEKLKRKPPSTNAGGGQGHELTCPSCDSYEKKPPKEFLERFKSLLQKMIHQHLSSRTHGSEDS

SEQ ID NO: 461 - protein, 133 aa, synthetic construct
QGQDFHMIRMRQLIDIVDQLKNYVNDLVPEFLPAPEDVETNCEWSAFSCFQKAQLKSANTGNNERIINVSIKKLARKPPSTNAGGGQGHELTCPSCDSYEKKPPKEFLERFKSLLQKMIHQHLSSRTHGSEDS

SEQ ID NO: 462 - protein, 133 aa, synthetic construct
QGQDFHMIRMRQLIDIVDQLKNYVNDLVPEFLPAPEDVETNCEWSAFSCFQKAQLKSANTGNNERIINVSIKKLERKPPSTNAGGGQGHELTCPSCDSYEKKPPKEFLERFKSLLQKMIHQHLSSRTHGSEDS

SEQ ID NO: 463 - protein, 133 aa, synthetic construct
QGQDRHMIRMRQLIDIVDQLKNYVNDLVPEFLPAPEDVETNCEWSAFSCFQKAQLKSANTGNNERIINVSIKALKFKPPSTNAGGGQGHELTCPSCDSYEKKPPKEFLERFKSLLQKMIHQHLSSRTHGSEDS

SEQ ID NO: 464 - protein, 133 aa, synthetic construct
QGQDRHMIRMRQLIDIVDQLKNYVNDLVPEFLPAPEDVETNCEWSAFSCFQKAQLKSANTGNNERIINVSIKELKFKPPSTNAGGGQGHELTCPSCDSYEKKPPKEFLERFKSLLQKMIHQHLSSRTHGSEDS

SEQ ID NO: 465 - protein, 133 aa, synthetic construct
QGQDRHMIAMRQLIDIVDQLKNYVNDLVPEFLPAPEDVETNCEWSAFSCFQKAQLKSANTGNNERIINVSIKKLKRKPPSTNAGGGQGHELTCPSCDSYEKKPPKEFLERFKSLLQKMIHQHLSSRTHGSEDS

SEQ ID NO: 466 - protein, 133 aa, synthetic construct
QGQDRHMIDMRQLIDIVDQLKNYVNDLVPEFLPAPEDVETNCEWSAFSCFQKAQLKSANTGNNERIINVSIKKLKRKPPSTNAGGGQGHELTCPSCDSYEKKPPKEFLERFKSLLQKMIHQHLSSRTHGSEDS

SEQ ID NO: 467 - protein, 133 aa, synthetic construct
QGQDRHMIEMRQLIDIVDQLKNYVNDLVPEFLPAPEDVETNCEWSAFSCFQKAQLKSANTGNNERIINVSIKKLKRKPPSTNAGGGQGHELTCPSCDSYEKKPPKEFLERFKSLLQKMIHQHLSSRTHGSEDS

SEQ ID NO: 468 - protein, 133 aa, synthetic construct
QGQDRHMIHMRQLIDIVDQLKNYVNDLVPEFLPAPEDVETNCEWSAFSCFQKAQLKSANTGNNERIINVSIKKLKRKPPSTNAGGGQGHELTCPSCDSYEKKPPKEFLERFKSLLQKMIHQHLSSRTHGSEDS

SEQ ID NO: 469 - protein, 133 aa, synthetic construct
QGQDRHMISMRQLIDIVDQLKNYVNDLVPEFLPAPEDVETNCEWSAFSCFQKAQLKSANTGNNERIINVSIKKLKRKPPSTNAGGGQGHELTCPSCDSYEKKPPKEFLERFKSLLQKMIHQHLSSRTHGSEDS

SEQ ID NO: 470 - protein, 133 aa, synthetic construct
QGQDRHMITMRQLIDIVDQLKNYVNDLVPEFLPAPEDVETNCEWSAFSCFQKAQLKSANTGNNERIINVSIKKLKRKPPSTNAGGGQGHELTCPSCDSYEKKPPKEFLERFKSLLQKMIHQHLSSRTHGSEDS

SEQ ID NO: 471 - protein, 133 aa, synthetic construct
QGQDRHMINMRQLIDIVDQLKNYVNDLVPEFLPAPEDVETNCEWSAFSCFQKAQLKSANTGNNERIINVSIKKLKRKPPSTNAGGGQGHELTCPSCDSYEKKPPKEFLERFKSLLQKMIHQHLSSRTHGSEDS

SEQ ID NO: 472 - protein, 133 aa, synthetic construct
QGQDRHMIGMRQLIDIVDQLKNYVNDLVPEFLPAPEDVETNCEWSAFSCFQKAQLKSANTGNNERIINVSIKKLKRKPPSTNAGGGQGHELTCPSCDSYEKKPPKEFLERFKSLLQKMIHQHLSSRTHGSEDS

SEQ ID NO: 473 - protein, 133 aa, synthetic construct
QGQDRHMIVMRQLIDIVDQLKNYVNDLVPEFLPAPEDVETNCEWSAFSCFQKAQLKSANTGNNERIINVSIKKLKRKPPSTNAGGGQGHELTCPSCDSYEKKPPKEFLERFKSLLQKMIHQHLSSRTHGSEDS

SEQ ID NO: 474 - protein, 133 aa, synthetic construct
QGQDRHMIIMRQLIDIVDQLKNYVNDLVPEFLPAPEDVETNCEWSAFSCFQKAQLKSANTGNNERIINVSIKKLKRKPPSTNAGGGQGHELTCPSCDSYEKKPPKEFLERFKSLLQKMIHQHLSSRTHGSEDS

SEQ ID NO: 475 - protein, 133 aa, synthetic construct
QGQDRHMILMRQLIDIVDQLKNYVNDLVPEFLPAPEDVETNCEWSAFSCFQKAQLKSANTGNNERIINVSIKKLKRKPPSTNAGGGQGHELTCPSCDSYEKKPPKEFLERFKSLLQKMIHQHLSSRTHGSEDS

SEQ ID NO: 476 - protein, 133 aa, synthetic construct
QGQDRHMIYMRQLIDIVDQLKNYVNDLVPEFLPAPEDVETNCEWSAFSCFQKAQLKSANTGNNERIINVSIKKLKRKPPSTNAGGGQGHELTCPSCDSYEKKPPKEFLERFKSLLQKMIHQHLSSRTHGSEDS

SEQ ID NO: 477 - protein, 133 aa, synthetic construct
QGQDRHMIRMRQLIDIVDQLKNYVNDLVPEFLPAPEDVETNCEWSAFSCFQKAQLKSANTGNNERIINVSIAKLKFKPPSTNAGGGQGHELTCPSCDSYEKKPPKEFLERFKSLLQKMIHQHLSSRTHGSEDS

SEQ ID NO: 478 - protein, 133 aa, synthetic construct
QGQDRHMIRMRQLIDIVDQLKNYVNDLVPEFLPAPEDVETNCEWSAFSCFQKAQLKSANTGNNERIINVSIKKLAFKPPSTNAGGGQGHELTCPSCDSYEKKPPKEFLERFKSLLQKMIHQHLSSRTHGSEDS

SEQ ID NO: 479 - protein, 133 aa, synthetic construct
QGQDRHMIRMRQLIDIVDQLKNYVNDLVPEFLPAPEDVETNCEWSAFSCFQKAQLKSANTGNNERIINVSIKKLKFAPPSTNAGGGQGHELTCPSCDSYEKKPPKEFLERFKSLLQKMIHQHLSSRTHGSEDS

SEQ ID NO: 480 - protein, 133 aa, synthetic construct
QGQDRHMIRMRQLIDIVDQLKNYVNDLVPEFLPAPEDVETNCEWSAFSCFQKAQLKSANTGNNERIINVSIKKLEFKPPSTNAGGGQGHELTCPSCDSYEKKPPKEFLERFKSLLQKMIHQHLSSRTHGSEDS

SEQ ID NO: 481 - protein, 133 aa, synthetic construct
QGQDRHMIRMRQLIDIIDQLKNYVNDLVPEFLPAPEDVETNCEWSAFSCFQKAQLKSANTGNNERIINVSIKKIKRKPPSTNAGGGQGHELTCPSCDSYEKKPPKEFLERFKSLLQKMIHQHLSSRTHGSEDS

SEQ ID NO: 482 - protein, 133 aa, synthetic construct
QGQDRHMIRMRQLIDAVDQLKNYVNDLVPEFLPAPEDVETNCEWSAFSCFQKAQLKSANTGNNERIINVSIKKFKRKPPSTNAGGGQGHELTCPSCDSYEKKPPKEFLERFKSLLQKMIHQHLSSRTHGSEDS

SEQ ID NO: 483 - protein, 133 aa, synthetic construct
QGQDRHMIRMRQLIDSIDQLKNYVNDLVPEFLPAPEDVETNCEWSAFSCFQKAQLKSANTGNNERIINVSIKKVKRKPPSTNAGGGQGHELTCPSCDSYEKKPPKEFLERFKSLLQKMIHQHLSSRTHGSEDS

SEQ ID NO: 484 - protein, 133 aa, synthetic construct
QGQDRHMIRMRQLIDRIDQLKNYVNDLVPEFLPAPEDVETNCEWSAFSCFQKAQLKSANTGNNERIINVSIKKIKRKPPSTNAGGGQGHELTCPSCDSYEKKPPKEFLERFKSLLQKMIHQHLSSRTHGSEDS

SEQ ID NO: 485 - protein, 133 aa, synthetic construct
QGQDRHMIRMRQFIDAADQLKNYVNDLVPEFLPAPEDVETNCEWSAFSCFQKAQLKSANTGNNERIINVSIKKMKRKPPSTNAGGGQGHELTCPSCDSYEKKPPKEFLERFKSLLQKMIHQHLSSRTHGSEDS

SEQ ID NO: 486 - protein, 133 aa, synthetic construct
QGQDRHMIRMRQRIDAIDQLKNYVNDLVPEFLPAPEDVETNCEWSAFSCFQKAQLKSANTGNNERIINVSIKKIKRKPPSTNAGGGQGHELTCPSCDSYEKKPPKEFLERFKSLLQKMIHQHLSSRTHGSEDS

SEQ ID NO: 487 - protein, 133 aa, synthetic construct
QGQDRHMIRMRQLIDIVDQLKNYVNDLVPEFLPAPEDVETNCEWSAFSCFQKAQLKSANTGNNERIINVSIKKLKEKPPGGEGGGGGGGEGGCPSCDSYEKKPPKEFLERFKSLLQKMIHQHLSSRTHGSEDS

SEQ ID NO: 488 - protein, 133 aa, synthetic construct
QGQDRHMIRMRQLIDIVDQLKNYVNDLVPEFLPAPEDVETNCEWSAFSCFQKAQLKSANTGNNERIINVSIKKLKFKPPGGEGGGGGGGEGGCPSCDSYEKKPPKEFLERFKSLLQKMIHQHLSSRTHGSEDS

SEQ ID NO: 489 - protein, 133 aa, synthetic construct
QGQDRHMIRMRQLIDIVDQLKNYVNDLVPEFLPAPEDVETNCEWSAFSCFQKAQLKSANTGNNERIINVSIKKLKQKPPGGEGGGGGGGEGGCPSCDSYEKKPPKEFLERFKSLLQKMIHQHLSSRTHGSEDS

SEQ ID NO: 490 - protein, 133 aa, synthetic construct
QGQDRHMISMRQLIDIVDQLKNYVNDLVPEFLPAPEDVETNCEWSAFSCFQKAQLKSANTGNNERIINVSIKKLKRKPPGGEGGGGGGGEGGCPSCDSYEKKPPKEFLERFKSLLQKMIHQHLSSRTHGSEDS

SEQ ID NO: 491 - protein, 133 aa, synthetic construct
QGQDRHMIGMRQLIDIVDQLKNYVNDLVPEFLPAPEDVETNCEWSAFSCFQKAQLKSANTGNNERIINVSIKKLKRKPPGGEGGGGGGGEGGCPSCDSYEKKPPKEFLERFKSLLQKMIHQHLSSRTHGSEDS

SEQ ID NO: 492 - protein, 133 aa, synthetic construct
QGQDRHMIRMDQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 493 - protein, 133 aa, synthetic construct
QGQDRHMIRMEQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 494 - protein, 133 aa, synthetic construct
QGQDRHMIRMRQLDDIVAQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQAMIHQHLSSRTHGSEDS

SEQ ID NO: 495 - protein, 133 aa, synthetic construct
QGQDRHMIRMRQLIDIVDQLKNYVNDLVPEFLPAPEDVETNCEWSAFSCFQKAQLKSANTGNNERIINVSIKKLKEKPPSTNAGRRQKHRLTCPSCDSYEKKPPKEFLERFKSLLQKMIHQHLSSRTHGSEDS

SEQ ID NO: 496 - protein, 133 aa, synthetic construct
QGQDFHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 497 - protein, 133 aa, synthetic construct
QGQDRHMIRMRQLIDIVDQLKNYVNDLVPEFLPAPEDVETNCEWSAFSCFQKAQLKSANTGNNERIINVSIKKLKFKPPSTNAGRRQKHRLTCPSCDSYEKKPPKEFLERFKSLLQKMIHQHLSSRTHGSEDS

SEQ ID NO: 498 - protein, 133 aa, synthetic construct
QGQDRHMERMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 499 - protein, 133 aa, synthetic construct
QGQDAHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 500 - protein, 133 aa, synthetic construct
QGQDEHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 501 - protein, 133 aa, synthetic construct
QGQDSHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 502 - protein, 133 aa, synthetic construct
QGQDTHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 503 - protein, 133 aa, synthetic construct
QGQDNHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 504 - protein, 133 aa, synthetic construct
QGQDQHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 505 - protein, 133 aa, synthetic construct
QGQDVHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 506 - protein, 133 aa, synthetic construct
QGQDIHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 507 - protein, 133 aa, synthetic construct
QGQDLHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 508 - protein, 133 aa, synthetic construct
QGQDYHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 509 - protein, 133 aa, synthetic construct
QGQDRHMIRMRQLIDIVDQLKNYVNDLVPEFLPAPEDVETNCEWSAFSCFQKAQLKSANTGNNERIINVSIKKLKAKPPSTNAGRRQKHRLTCPSCDSYEKKPPKEFLERFKSLLQKMIHQHLSSRTHGSEDS

SEQ ID NO: 510 - protein, 133 aa, synthetic construct
QGQDRHMIRMRQLIDIVDQLKNYVNDLVPEFLPAPEDVETNCEWSAFSCFQKAQLKSANTGNNERIINVSIKKLKNKPPSTNAGRRQKHRLTCPSCDSYEKKPPKEFLERFKSLLQKMIHQHLSSRTHGSEDS

SEQ ID NO: 511 - protein, 133 aa, synthetic construct
QGQDRHMIRMRQLIDIVDQLKNYVNDLVPEFLPAPEDVETNCEWSAFSCFQKAQLKSANTGNNERIINVSIKKLKDKPPSTNAGRRQKHRLTCPSCDSYEKKPPKEFLERFKSLLQKMIHQHLSSRTHGSEDS

SEQ ID NO: 512 - protein, 133 aa, synthetic construct
QGQDRHMIRMRQLIDIVDQLKNYVNDLVPEFLPAPEDVETNCEWSAFSCFQKAQLKSANTGNNERIINVSIKKLKSKPPSTNAGRRQKHRLTCPSCDSYEKKPPKEFLERFKSLLQKMIHQHLSSRTHGSEDS

SEQ ID NO: 513 - protein, 133 aa, synthetic construct
QGQDRHMIRMRQLIDIVDQLKNYVNDLVPEFLPAPEDVETNCEWSAFSCFQKAQLKSANTGNNERIINVSIKKLKTKPPSTNAGRRQKHRLTCPSCDSYEKKPPKEFLERFKSLLQKMIHQHLSSRTHGSEDS

SEQ ID NO: 514 - protein, 133 aa, synthetic construct
QGQDRHMIRMRQLIDIVDQLKNYVNDLVPEFLPAPEDVETNCEWSAFSCFQKAQLKSANTGNNERIINVSIKKLKQKPPSTNAGRRQKHRLTCPSCDSYEKKPPKEFLERFKSLLQKMIHQHLSSRTHGSEDS

SEQ ID NO: 515 - protein, 133 aa, synthetic construct
QGQDRHMIRMRQLIDIVDQLKNYVNDLVPEFLPAPEDVETNCEWSAFSCFQKAQLKSANTGNNERIINVSIKKLKVKPPSTNAGRRQKHRLTCPSCDSYEKKPPKEFLERFKSLLQKMIHQHLSSRTHGSEDS

SEQ ID NO: 516 - protein, 133 aa, synthetic construct
QGQDRHMIRMRQLIDIVDQLKNYVNDLVPEFLPAPEDVETNCEWSAFSCFQKAQLKSANTGNNERIINVSIKKLKIKPPSTNAGRRQKHRLTCPSCDSYEKKPPKEFLERFKSLLQKMIHQHLSSRTHGSEDS

SEQ ID NO: 517 - protein, 133 aa, synthetic construct
QGQDRHMIRMRQLIDIVDQLKNYVNDLVPEFLPAPEDVETNCEWSAFSCFQKAQLKSANTGNNERIINVSIKKLKLKPPSTNAGRRQKHRLTCPSCDSYEKKPPKEFLERFKSLLQKMIHQHLSSRTHGSEDS

SEQ ID NO: 518 - protein, 133 aa, synthetic construct
QGQDRHMIRMRQLIDIVDQLKNYVNDLVPEFLPAPEDVETNCEWSAFSCFQKAQLKSANTGNNERIINVSIKKLKYKPPSTNAGRRQKHRLTCPSCDSYEKKPPKEFLERFKSLLQKMIHQHLSSRTHGSEDS

SEQ ID NO: 519 - protein, 133 aa, synthetic construct
QGQDRHMIRMRQLIDIVDQLKNYVNDLVPEFLPAPEDVETNCEWSAFSCFQKAQLKSANTGNNERIINVSIKKLKRAPPSTNAGRRQKHRLTCPSCDSYEKKPPKEFLERFKSLLQKMIHQHLSSRTHGSEDS

SEQ ID NO: 520 - protein, 133 aa, synthetic construct
QGQDRHMIRMRQLIDIVDQLKNYVNDLVPEFLPAPEDVETNCEWSAFSCFQKAQLKSANTGNNERIINVSIKKLKREPPSTNAGRRQKHRLTCPSCDSYEKKPPKEFLERFKSLLQKMIHQHLSSRTHGSEDS

SEQ ID NO: 521 - protein, 133 aa, synthetic construct
QGQDRHMIRMRQLIDIVDQLKNYVNDLVPEFLPAPEDVETNCEWSAFSCFQKAQLKSANTGNNERIINVSIAKLKRKPPSTNAGRRQKHRLTCPSCDSYEKKPPKEFLERFKSLLQKMIHQHLSSRTHGSEDS

SEQ ID NO: 522 - protein, 133 aa, synthetic construct
QGQDRHMIRMRQLIDIVDQLKNYVNDLVPEFLPAPEDVETNCEWSAFSCFQKAQLKSANTGNNERIINVSIEKLKRKPPSTNAGRRQKHRLTCPSCDSYEKKPPKEFLERFKSLLQKMIHQHLSSRTHGSEDS

SEQ ID NO: 523 - protein, 133 aa, synthetic construct
QGQDRHMIRMRQLIDIVDQLKNYVNDLVPEFLPAPEDVETNCEWSAFSCFQKAQLKSANTGNNERIINVSIKKLARKPPSTNAGRRQKHRLTCPSCDSYEKKPPKEFLERFKSLLQKMIHQHLSSRTHGSEDS

SEQ ID NO: 524 - protein, 133 aa, synthetic construct
QGQDRHMIRMRQLIDIVDQLKNYVNDLVPEFLPAPEDVETNCEWSAFSCFQKAQLKSANTGNNERIINVSIKKLERKPPSTNAGRRQKHRLTCPSCDSYEKKPPKEFLERFKSLLQKMIHQHLSSRTHGSEDS

SEQ ID NO: 525 - protein, 133 aa, synthetic construct
QGQDRHMIRMRQLIDIVDQLKNYVNDLVPEFLPAPEDVETNCEWSAFSCFQKAQLKSANTGNNERIINVSIKALKRKPPSTNAGRRQKHRLTCPSCDSYEKKPPKEFLERFKSLLQKMIHQHLSSRTHGSEDS

SEQ ID NO: 526 - protein, 133 aa, synthetic construct
QGQDRHMIRMRQLIDIVDQLKNYVNDLVPEFLPAPEDVETNCEWSAFSCFQKAQLKSANTGNNERIINVSIKELKRKPPSTNAGRRQKHRLTCPSCDSYEKKPPKEFLERFKSLLQKMIHQHLSSRTHGSEDS

SEQ ID NO: 527 - protein, 133 aa, synthetic construct
QGQDFHMIRMRQLIDIVDQLKNYVNDLVPEFLPAPEDVETNCEWSAFSCFQKAQLKSANTGNNERIINVSIKKLKRAPPSTNAGRRQKHRLTCPSCDSYEKKPPKEFLERFKSLLQKMIHQHLSSRTHGSEDS

SEQ ID NO: 528 - protein, 133 aa, synthetic construct
QGQDFHMIRMRQLIDIVDQLKNYVNDLVPEFLPAPEDVETNCEWSAFSCFQKAQLKSANTGNNERIINVSIKKLKREPPSTNAGRRQKHRLTCPSCDSYEKKPPKEFLERFKSLLQKMIHQHLSSRTHGSEDS

SEQ ID NO: 529 - protein, 133 aa, synthetic construct
QGQDFHMIRMRQLIDIVDQLKNYVNDLVPEFLPAPEDVETNCEWSAFSCFQKAQLKSANTGNNERIINVSIAKLKRKPPSTNAGRRQKHRLTCPSCDSYEKKPPKEFLERFKSLLQKMIHQHLSSRTHGSEDS

SEQ ID NO: 530 - protein, 133 aa, synthetic construct
QGQDFHMIRMRQLIDIVDQLKNYVNDLVPEFLPAPEDVETNCEWSAFSCFQKAQLKSANTGNNERIINVSIEKLKRKPPSTNAGRRQKHRLTCPSCDSYEKKPPKEFLERFKSLLQKMIHQHLSSRTHGSEDS

SEQ ID NO: 531 - protein, 133 aa, synthetic construct
QGQDFHMIRMRQLIDIVDQLKNYVNDLVPEFLPAPEDVETNCEWSAFSCFQKAQLKSANTGNNERIINVSIKKLARKPPSTNAGRRQKHRLTCPSCDSYEKKPPKEFLERFKSLLQKMIHQHLSSRTHGSEDS

SEQ ID NO: 532 - protein, 133 aa, synthetic construct
QGQDFHMIRMRQLIDIVDQLKNYVNDLVPEFLPAPEDVETNCEWSAFSCFQKAQLKSANTGNNERIINVSIKKLERKPPSTNAGRRQKHRLTCPSCDSYEKKPPKEFLERFKSLLQKMIHQHLSSRTHGSEDS

SEQ ID NO: 533 - protein, 133 aa, synthetic construct
QGQDRHMIRMRQLIDIVDQLKNYVNDLVPEFLPAPEDVETNCEWSAFSCFQKAQLKSANTGNNERIINVSIKALKFKPPSTNAGRRQKHRLTCPSCDSYEKKPPKEFLERFKSLLQKMIHQHLSSRTHGSEDS

SEQ ID NO: 534 - protein, 133 aa, synthetic construct
QGQDRHMIRMRQLIDIVDQLKNYVNDLVPEFLPAPEDVETNCEWSAFSCFQKAQLKSANTGNNERIINVSIKELKFKPPSTNAGRRQKHRLTCPSCDSYEKKPPKEFLERFKSLLQKMIHQHLSSRTHGSEDS

SEQ ID NO: 535 - protein, 133 aa, synthetic construct
QGQDRHMIAMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 536 - protein, 133 aa, synthetic construct
QGQDRHMIDMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 537 - protein, 133 aa, synthetic construct
QGQDRHMIEMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 538 - protein, 133 aa, synthetic construct
QGQDRHMIHMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 539 - protein, 133 aa, synthetic construct
QGQDRHMISMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 540 - protein, 133 aa, synthetic construct
QGQDRHMITMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 541 - protein, 133 aa, synthetic construct
QGQDRHMINMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 542 - protein, 133 aa, synthetic construct
QGQDRHMIGMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 543 - protein, 133 aa, synthetic construct
QGQDRHMIVMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 544 - protein, 133 aa, synthetic construct
QGQDRHMIIMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 545 - protein, 133 aa, synthetic construct
QGQDRHMILMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 546 - protein, 133 aa, synthetic construct
QGQDRHMIYMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 547 - protein, 133 aa, synthetic construct
QGQDRHMIRMRQLIDIVDQLKNYVNDLVPEFLPAPEDVETNCEWSAFSCFQKAQLKSANTGNNERIINVSIAKLKFKPPSTNAGRRQKHRLTCPSCDSYEKKPPKEFLERFKSLLQKMIHQHLSSRTHGSEDS

SEQ ID NO: 548 - protein, 133 aa, synthetic construct
QGQDRHMIRMRQLIDIVDQLKNYVNDLVPEFLPAPEDVETNCEWSAFSCFQKAQLKSANTGNNERIINVSIKKLAFKPPSTNAGRRQKHRLTCPSCDSYEKKPPKEFLERFKSLLQKMIHQHLSSRTHGSEDS

SEQ ID NO: 549 - protein, 133 aa, synthetic construct
QGQDRHMIRMRQLIDIVDQLKNYVNDLVPEFLPAPEDVETNCEWSAFSCFQKAQLKSANTGNNERIINVSIKKLKFAPPSTNAGRRQKHRLTCPSCDSYEKKPPKEFLERFKSLLQKMIHQHLSSRTHGSEDS

SEQ ID NO: 550 - protein, 133 aa, synthetic construct
QGQDRHMIRMRQLIDIVDQLKNYVNDLVPEFLPAPEDVETNCEWSAFSCFQKAQLKSANTGNNERIINVSIKKLEFKPPSTNAGRRQKHRLTCPSCDSYEKKPPKEFLERFKSLLQKMIHQHLSSRTHGSEDS

SEQ ID NO: 551 - protein, 133 aa, synthetic construct
  VARIANT 5: note = F, A, E, S, T, N, Q, V, I, L, Y or R
  VARIANT 8: note = E or I
  VARIANT 9: note = A, D, E, H, S, T, N, G, V, I, L, Y or R
  VARIANT 11: note = D, E or R
  VARIANT 13: note = F, R or L
  VARIANT 14: note = D or I
  VARIANT 16: note = A, S, R or I
  VARIANT 17: note = I, A or V
  VARIANT 18: note = A or D
  VARIANT 72: note = A, E or K
  VARIANT 73: note = A, E or K
  VARIANT 74: note = I, F, M or L
  VARIANT 75: note = A, K or E
  VARIANT 76: note = E, F, A, N, D, S, T, Q, V, I, L, Y, M or R
  VARIANT 77: note = A, E or K
  VARIANT 117: note = A or K
QGQDXHMXXMXQXXDXXXQLKNYVNDLVPEFLPAPEDVETNCEWSAFSCFQKAQLKSANTGNNERIINVSIXXXXXXPPSTNAGRRQKHRLTCPSCDSYEKKPPKEFLERFKSLLQXMIHQHLSSRTHGSEDS

SEQ ID NO: 552 - protein, 133 aa, synthetic construct
  VARIANT 5: note = R, A, D, E, S, T, N, Q, V, I, L, Y, or F
  VARIANT 8: note = I, Q, H, E
  VARIANT 9: note = R, A, D, E, S, T, N, Q, V, I, L, Y, or F
  VARIANT 11: note = R, D or E
  VARIANT 12: note = Q, L, I, or Y
  VARIANT 14: note = I, D or E
  VARIANT 15: note = D, R, K, H, L, Y, or F
  VARIANT 18: note = D, A, K, or R
  VARIANT 19: note = Q, L, or Y
  VARIANT 23: note = Y or E
  VARIANT 65: note = R, G, S, E, D, or A
  VARIANT 70: note = S, H, Y, L, V, or F
  VARIANT 72: note = K, G, S, E, D, or A
  VARIANT 73: note = K, A, Y, L, F, G, S, T, E, A, or D
  VARIANT 75: note = K, G, S, E, D, or A
  VARIANT 76: note = R, A, D, E, S, T, N, Q, V, I, L, Y, or F
  VARIANT 77: note = K, G, S, E, D, or A
  VARIANT 80: note = S, H, A, G, E, or D
  VARIANT 116: note = Q or Y
  VARIANT 117: note = K, A, D, or E
QGQDXHMXXMXXLXXIVXXLKNXVNDLVPEFLPAPEDVETNCEWSAFSCFQKAQLKSANTGNNEXIINVXIXXLXXXPPXTNAGRRQKHRLTCPSCDSYEKKPPKEFLERFKSLLXXMIHQHLSSRTHGSEDS

SEQ ID NO: 553 - protein, 133 aa, synthetic construct
  VARIANT 56: note = A, E or K
  VARIANT 75: note = A, E or K
  VARIANT 80: note = G or S
  VARIANT 81: note = G or T
  VARIANT 82: note = G, E or N
  VARIANT 83: note = G, S, E or A
  VARIANT 84: note = E or G
  VARIANT 85: note = G, E, S or R
  VARIANT 86: note = G, E or R
  VARIANT 87: note = S, G, E or Q
  VARIANT 88: note = G or K
  VARIANT 89: note = G, S, or H
  VARIANT 90: note = A, E, S, G or R
  VARIANT 91: note = S, G or L
  VARIANT 92: note = G, S or T
QGQDRHMIRMRQLIDIVDQLKNYVNDLVPEFLPAPEDVETNCEWSAFSCFQKAQLXSANTGNNERIINVSIKKLXRKPPXXXXXXXXXXXXXCPSCDSYEKKPPKEFLERFKSLLQKMIHQHLSSRTHGSEDS

SEQ ID NO: 554 - protein, 133 aa, synthetic construct
  VARIANT 80: note = G or S
  VARIANT 81: note = G or T
  VARIANT 82: note = G, E or N
  VARIANT 83: note = G, S, E or A
  VARIANT 85: note = G, E, S or R
  VARIANT 86: note = G, E or R
  VARIANT 87: note = S, G, E or Q
  VARIANT 88: note = G or K
  VARIANT 89: note = G, S or H
  VARIANT 90: note = A, E, S, G or R
  VARIANT 91: note = S, G or L
  VARIANT 92: note = G, S or T
QGQDRHMIRMRQLIDIVDQLKNYVNDLVPEFLPAPEDVETNCEWSAFSCFQKAQLKSANTGNNERIINVSIKKLKRKPPXXXXGXXXXXXXXCPSCDSYEKKPPKEFLERFKSLLQKMIHQHLSSRTHGSEDS

SEQ ID NO: 555 - protein, 17 aa, synthetic construct
  VARIANT 3: note = G or S
  VARIANT 4: note = G or T
  VARIANT 5: note = G, E or N
  VARIANT 6: note = G, S, E or A
  VARIANT 8: note = G, E, S or R
  VARIANT 9: note = G, E or R
  VARIANT 10: note = S, G, E or Q
  VARIANT 11: note = G or K
  VARIANT 12: note = G, S or H
  VARIANT 13: note = A, E, S, G or R
  VARIANT 14: note = S, G or L
  VARIANT 15: note = G, S or T
PPXXXXGXXXXXXXXCP

SEQ ID NO: 556 - protein, 133 aa, synthetic construct
  VARIANT 5: note = F, A, E, S, T, N, Q, V, I, L, Y or R
  VARIANT 8: note = E or I
  VARIANT 9: note = A, D, E, H, S, T, N, G, V, I, L, Y or R
  VARIANT 11: note = D, E or R
  VARIANT 13: note = F, R or L
  VARIANT 14: note = D or I
  VARIANT 16: note = A, S, R or I
  VARIANT 17: note = I, A or V
  VARIANT 18: note = A or D
  VARIANT 56: note = A, E or K
  VARIANT 72: note = A, E or K
  VARIANT 73: note = A, E or K
  VARIANT 74: note = I, F, M or L
  VARIANT 75: note = A, K or E
  VARIANT 76: note = E, F, A, N, D, S, T, Q, V, I, L, Y, M or R
  VARIANT 77: note = A, E or K
  VARIANT 80: note = G or S
  VARIANT 81: note = G or T
  VARIANT 82: note = G, E or N
  VARIANT 83: note = G, S, E or A
  VARIANT 84: note = E or G
  VARIANT 85: note = G, E, S or R
  VARIANT 86: note = G, E or R
  VARIANT 87: note = S, G, E or Q
  VARIANT 88: note = G or K
  VARIANT 89: note = G, S or H
  VARIANT 90: note = A, E, S, G or R
  VARIANT 91: note = S, G or L
  VARIANT 92: note = G, S or T
  VARIANT 117: note = A or K
QGQDXHMXXMXQXXDXXXQLKNYVNDLVPEFLPAPEDVETNCEWSAFSCFQKAQLXSANTGNNERIINVSIXXXXXXPPXXXXXXXXXXXXXCPSCDSYEKKPPKEFLERFKSLLQXMIHQHLSSRTHGSEDS

SEQ ID NO: 557 - protein, 214 aa, synthetic construct; identical to SEQ ID NO: 310

SEQ ID NO: 558 - protein, 597 aa, synthetic construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSQGQDRHMIRMRQLIDIVDQLKNYVNDLVPEFLPAPEDVETNCEWSAFSCFQKAQLKSANTGNNERIINVSIKKLKEKPPGGEGGGGGGGEGGCPSCDSYEKKPPKEFLERFKSLLQKMIHQHLSSRTHGSEDS

SEQ ID NO: 559 - protein, 448 aa, synthetic construct; identical to SEQ ID NO: 312

SEQ ID NO: 560 - protein, 449 aa, synthetic construct; identical to SEQ ID NO: 313

SEQ ID NO: 561 - protein, 214 aa, synthetic construct; identical to SEQ ID NO: 310

SEQ ID NO: 562 - protein, 597 aa, synthetic construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSQGQDRHMIRMRQLIDIVDQLKNYVNDLVPEFLPAPEDVETNCEWSAFSCFQKAQLKSANTGNNERIINVSIKKLKFKPPGGEGGGGGGGEGGCPSCDSYEKKPPKEFLERFKSLLQKMIHQHLSSRTHGSEDS

SEQ ID NO: 563 - protein, 448 aa, synthetic construct; identical to SEQ ID NO: 312

SEQ ID NO: 564 - protein, 449 aa, synthetic construct; identical to SEQ ID NO: 313

SEQ ID NO: 565 - protein, 214 aa, synthetic construct; identical to SEQ ID NO: 310

SEQ ID NO: 566 - protein, 597 aa, synthetic construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSQGQDRHMIRMRQLIDIVDQLKNYVNDLVPEFLPAPEDVETNCEWSAFSCFQKAQLKSANTGNNERIINVSIKKLKQKPPGGEGGGGGGGEGGCPSCDSYEKKPPKEFLERFKSLLQKMIHQHLSSRTHGSEDS

SEQ ID NO: 567 - protein, 448 aa, synthetic construct; identical to SEQ ID NO: 312

SEQ ID NO: 568 - protein, 449 aa, synthetic construct; identical to SEQ ID NO: 313

SEQ ID NO: 569 - protein, 214 aa, synthetic construct; identical to SEQ ID NO: 310

SEQ ID NO: 570 - protein, 597 aa, synthetic construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSQGQDRHMISMRQLIDIVDQLKNYVNDLVPEFLPAPEDVETNCEWSAFSCFQKAQLKSANTGNNERIINVSIKKLKRKPPGGEGGGGGGGEGGCPSCDSYEKKPPKEFLERFKSLLQKMIHQHLSSRTHGSEDS

SEQ ID NO: 571 - protein, 448 aa, synthetic construct; identical to SEQ ID NO: 312

SEQ ID NO: 572 - protein, 449 aa, synthetic construct; identical to SEQ ID NO: 313

SEQ ID NO: 573 - protein, 214 aa, synthetic construct; identical to SEQ ID NO: 310

SEQ ID NO: 574 - protein, 597 aa, synthetic construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSQGQDRHMIGMRQLIDIVDQLKNYVNDLVPEFLPAPEDVETNCEWSAFSCFQKAQLKSANTGNNERIINVSIKKLKRKPPGGEGGGGGGGEGGCPSCDSYEKKPPKEFLERFKSLLQKMIHQHLSSRTHGSEDS

SEQ ID NO: 575 - protein, 448 aa, synthetic construct; identical to SEQ ID NO: 312

SEQ ID NO: 576 - protein, 449 aa, synthetic construct; identical to SEQ ID NO: 313

SEQ ID NO: 577 - protein, 215 aa, synthetic construct; identical to SEQ ID NO: 302

SEQ ID NO: 578 - protein, 599 aa, synthetic construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSQGQDRHMIRMRQLIDIVDQLKNYVNDLVPEFLPAPEDVETNCEWSAFSCFQKAQLKSANTGNNERIINVSIKKLKEKPPGGEGGGGGGGEGGCPSCDSYEKKPPKEFLERFKSLLQKMIHQHLSSRTHGSEDS

SEQ ID NO: 579 - protein, 450 aa, synthetic construct; identical to SEQ ID NO: 308

SEQ ID NO: 580 - protein, 451 aa, synthetic construct; identical to SEQ ID NO: 309

SEQ ID NO: 581 - protein, 215 aa, synthetic construct; identical to SEQ ID NO: 302

SEQ ID NO: 582 - protein, 599 aa, synthetic construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSQGQDRHMIRMRQLIDIVDQLKNYVNDLVPEFLPAPEDVETNCEWSAFSCFQKAQLKSANTGNNERIINVSIKKLKFKPPGGEGGGGGGGEGGCPSCDSYEKKPPKEFLERFKSLLQKMIHQHLSSRTHGSEDS

SEQ ID NO: 583 - protein, 450 aa, synthetic construct; identical to SEQ ID NO: 308

SEQ ID NO: 584 - protein, 451 aa, synthetic construct; identical to SEQ ID NO: 309

SEQ ID NO: 585 - protein, 215 aa, synthetic construct; identical to SEQ ID NO: 302

SEQ ID NO: 586 - protein, 599 aa, synthetic construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSQGQDRHMIRMRQLIDIVDQLKNYVNDLVPEFLPAPEDVETNCEWSAFSCFQKAQLKSANTGNNERIINVSIKKLKQKPPGGEGGGGGGGEGGCPSCDSYEKKPPKEFLERFKSLLQKMIHQHLSSRTHGSEDS

SEQ ID NO: 587 - protein, 450 aa, synthetic construct; identical to SEQ ID NO: 308

SEQ ID NO: 588 - protein, 451 aa, synthetic construct; identical to SEQ ID NO: 309

SEQ ID NO: 589 - protein, 215 aa, synthetic construct; identical to SEQ ID NO: 302

SEQ ID NO: 590 - protein, 599 aa, synthetic construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSQGQDRHMIGMRQLIDIVDQLKNYVNDLVPEFLPAPEDVETNCEWSAFSCFQKAQLKSANTGNNERIINVSIKKLKRKPPGGEGGGGGGGEGGCPSCDSYEKKPPKEFLERFKSLLQKMIHQHLSSRTHGSEDS

SEQ ID NO: 591 - protein, 450 aa, synthetic construct; identical to SEQ ID NO: 308

SEQ ID NO: 592 - protein, 451 aa, synthetic construct; identical to SEQ ID NO: 309

SEQ ID NO: 593 - protein, 124 aa, synthetic construct
QGPDRLLIRLRHLIDIVEQLKIYENDLDPELLSAPQDVKGHCEHAAFACFQKAKLKPSNPGNNKTFIIDLVAQLEERLPAGEGGEGQEHIAKCPSCDSYEKRTPKEFLERLKWLLQKMIHQHLS

SEQ ID NO: 594 - protein, 149 aa, synthetic construct
APTSSSTSSSTAEAQQQQQQQQQQQQHLEQLLMDLQELLSRMENYRNLKLPEMLTAKFALPKQATELKDLQCLEDELGPLRHVLDLTQSKSFQLEDAENFISSIRVTVVKLKGSDNTFECQFDDESATVVDFLRRWIAFAQSIISTSPQ

(19) United States
(12) Patent Application Publication
(10) Pub. No.: US 2025/0339519 A1
(43) Pub. Date: Nov. 6, 2025

(54) COMBINATION OF CYTOKINE FUSION PROTEINS WITH CD8 ANTIGEN BINDING MOLECULES

(71) Applicant: Asher Biotherapeutics, Inc., South San Francisco, CA (US)

(72) Inventors: Ivana Djuretic, Pacifica, CA (US); Renee L. Greer, Pacifica, CA (US); Yik Andy Yeung, South San Francisco, CA (US)

(21) Appl. No.: 18/859,452

(22) PCT Filed: Apr. 26, 2023
(86) PCT No.: PCT/US2023/019989

§ 371 Date: Oct. 23, 2024

Related U.S. Application Data
(60) Provisional application No. 63/335,697, filed on Apr. 27, 2022.

(51) Int. Cl. A61K 39/395; A61K 38/20; A61K 39/00; A61P 35/00; C07K 14/54; C07K 14/55; C07K 16/28

(52) U.S. Cl. CPC A61K 39/39541; A61K 38/20; A61K 38/2013; A61P 35/00; C07K 14/54; C07K 14/55; C07K 16/2815; A61K 2039/505; C07K 2319/00

(57) ABSTRACT

Provided herein are anti-CD8 antigen binding molecules and fusion polypeptides with IL-2 or IL-21 comprising the CD8 antigen binding molecules for selectively modulating the function of CD8+ T cells over other immune cells. The present disclosure provides methods for using IL-2 and IL-21 fusion proteins in combination to treat a disease.

### CROSS-REFERENCE TO RELATED APPLICATIONS

This application is a 371 US National Phase of International Application No. PCT/US2023/019989, filed Apr. 26, 2023, which claims the benefit of U.S. Provisional Patent Application No. 63/335,697, filed Apr. 27, 2022, each of which is incorporated herein by reference in its entirety.

### BACKGROUND

CD8+ T cells expressing alpha beta T cell receptors are a large subset of major histocompatibility (MHC) class I-restricted T cells that mediate adaptive immunity to various pathogens and cancers. In addition, they can also be pathogenic and cause disease in certain autoimmune and inflammatory conditions. Modulating the function of CD8+ T cells, either by activating their function in the context of infection and cancer, or by inhibiting their function in the context of certain autoimmune diseases, can have therapeutic benefits.

CD8+ T cell activation and differentiation is in large part controlled by soluble immunomodulatory proteins such as cytokines. Biological activity of cytokines is mediated by binding to their respective cytokine receptors on the cell surface, typically with very high affinity, resulting in their ability to potently stimulate signal transduction downstream of their receptors triggering various cellular processes that regulate immune cell phenotype and function. Cytokines typically have pleiotropic effects, causing multiple downstream cellular events such as activation, proliferation, survival, apoptosis, and secretion of other immunomodulatory proteins. In addition, because their receptors are expressed on multiple immune cell subsets, cytokines act not only on CD8+ T cells but also on other immune and non-immune cells that express their receptors.

A challenge of cytokine immunotherapy is that in some cases, while activating immune cells to potentiate immune responses, the same cytokine can also activate counter-regulatory pathways as exemplified by IL-2 and IFNγ. These counter-regulatory pathways can activate regulatory T cell responses and inhibitory pathways. Accordingly, there remains a need for improved therapeutics for targeting CD8+ T cells without activating regulatory T cell responses and inhibitory pathways.

### SUMMARY OF THE DISCLOSURE

In some aspects, the disclosure provides a method of a treating a disease in a subject, the method comprising administering to the subject an effective amount of:
(a) an IL-21 fusion protein comprising (i) an antibody that specifically binds to human CD8, or antigen-binding fragment thereof; and (ii) an IL-21 polypeptide; and
(b) an IL-2 fusion protein comprising (i) an antibody that specifically binds human CD8, or antigen-binding fragment thereof; and (ii) an IL-2 polypeptide, thereby treating the subject.

In some aspects, the disclosure provides a method of a treating a disease in a subject who has received or is receiving treatment with an IL-21 fusion protein comprising an antibody that specifically binds human CD8, or antigen-binding fragment thereof, and an IL-21 polypeptide, the method comprising: administering to the subject an effective amount of an IL-2 fusion protein comprising (i) an antibody that specifically binds human CD8, or antigen-binding fragment thereof; and (ii) an IL-2 polypeptide, thereby treating the subject.

In some aspects, the disclosure provides a method of a treating a disease in a subject who has received or is receiving treatment with an IL-2 fusion protein comprising an antibody that specifically binds human CD8, or an antigen-binding fragment thereof, and an IL-2 polypeptide, the method comprising: administering to the subject an effective amount of an IL-21 fusion protein comprising (i) an antibody that specifically binds human CD8, or antigen-binding fragment thereof; and (ii) an IL-21 polypeptide, thereby treating the subject.

In some aspects, the disclosure provides a kit comprising one or more containers comprising (a) an IL-21 fusion protein comprising (i) an antibody that specifically binds to human CD8, or antigen-binding fragment thereof; and (ii) an IL-21 polypeptide; and (b) an IL-2 fusion protein comprising (i) an antibody that specifically binds human CD8, or antigen-binding fragment thereof; and (ii) an IL-2 polypeptide, and optionally a pharmaceutically acceptable carrier, and instructions for administering the IL-21 and IL-2 fusion proteins concurrently, sequentially, or simultaneously to a subject in need thereof.

In some aspects, the disclosure provides a kit comprising a container comprising an IL-21 fusion protein comprising an antibody that specifically binds to human CD8, or antigen-binding fragment thereof; and an IL-21 polypeptide, and optionally a pharmaceutically acceptable carrier, and instructions for administering the IL-21 fusion protein to a subject in need thereof that has received or is receiving treatment with an IL-2 fusion protein comprising an antibody that specifically binds human CD8, or antigen-binding fragment thereof; and an IL-2 polypeptide, thereby treating the subject.

In some aspects, the disclosure provides a kit comprising a container comprising an IL-2 fusion protein comprising an antibody that specifically binds to human CD8, or antigen-binding fragment thereof; and an IL-2 polypeptide, and optionally a pharmaceutically acceptable carrier, and instructions for administering the IL-2 fusion protein to a subject in need thereof that has received or is receiving treatment with an IL-21 fusion protein comprising an antibody that specifically binds human CD8, or antigen-binding fragment thereof; and an IL-21 polypeptide, thereby treating the subject.

In some aspects, the disclosure provides use of an IL-21 fusion protein comprising an antibody that specifically binds human CD8, or antigen-binding fragment thereof and an IL-21 polypeptide for administering to a subject in need thereof, wherein the subject has received or is receiving treatment with an IL-2 fusion protein comprising an antibody that specifically binds to human CD8, or antigen-binding fragment thereof; and an IL-2 polypeptide.

In some aspects, the disclosure provides use of an IL-2 fusion protein comprising an antibody that specifically binds human CD8, or antigen-binding fragment thereof and an IL-21 polypeptide for administering to a subject in need thereof, wherein the subject has received or is receiving treatment with an IL-21 fusion protein comprising an antibody that specifically binds to human CD8, or antigen-binding fragment thereof; and an IL-21 polypeptide.

In any of the foregoing or related aspects, the subject in need thereof has a disease. In some aspects, the disease is a cancer. In some aspects, the disease is an infectious disease.

In any of the foregoing or related aspects, treatment induces an immune response in the subject. In some aspects, the immune response is a T cell response.

In any of the foregoing or related aspects, treatment delays cancer progression in the subject. In some aspects, treatment reduces tumor volume in the subject. In some aspects, treatment reduces or inhibits tumor growth in the subject. In some aspects, treatment delays cancer progression, reduces tumor volume, and/or reduces or inhibits tumor growth in the subject more than treatment with either the IL-2 fusion protein or the IL-21 fusion protein alone in a subject.

In any of the foregoing or related aspects, the antibody or antigen-binding fragment thereof of the IL-21 and IL-2 fusion proteins are the same. In some aspects, the antibody or antigen-binding fragment thereof of the IL-21 and IL-2 fusion proteins are different. In some aspects, the antibody or antigen-binding fragment thereof of the IL-21 and IL-2 fusion proteins binds to a cell expressing a human CD8ab heterodimer on its surface with an EC50 that is less than 1000 nM. In some aspects, the antibody or antigen-binding fragment thereof of the IL-21 and IL-2 fusion proteins binds human CD8+ T cells.

In some aspects, the antibody or antigen-binding fragment thereof of the IL-21 and IL-2 fusion proteins bind different epitopes on CD8ab or CD8b. In some aspects, the antibody or antigen-binding fragment thereof of the IL-21 and IL-2 fusion proteins do not compete for binding to CD8ab or CD8b. In some aspects, the IL-21 fusion protein comprises a first antibody that specifically binds human CD8, or antigen-binding fragment thereof, and the IL-2 fusion protein comprises a second antibody that specifically binds human CD8, or antigen-binding fragment thereof, wherein the first and second antibodies bind different epitopes on CD8ab or CD8b. In some aspects, the first and second antibodies do not cross compete for binding to CD8ab or CD8b.

In some aspects, the antibody or antigen-binding fragment thereof of the IL-21 and IL-2 fusion proteins comprises a heavy chain variable (VH) domain and a light chain variable (VL) domain, wherein:
the VH domain comprises a CDR-H1 comprising the amino acid sequence of SEQ ID NO: 13, a CDR-H2 comprising the amino acid sequence of SEQ ID NO: 14, and a CDR-H3 comprising the amino acid sequence of SEQ ID NO: 15; and wherein the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 18;
the VH domain comprises a CDR-H1 comprising the amino acid sequence of SEQ ID NO: 19, a CDR-H2 comprising the amino acid sequence of SEQ ID NO: 20, and a CDR-H3 comprising the amino acid sequence of SEQ ID NO: 21; and wherein the VL domain comprises a CDR-L1 comprising the amino acid sequence of SEQ ID NO: 22, a CDR-L2 comprising the amino acid sequence of SEQ ID NO: 23, and a CDR-L3 comprising the amino acid sequence of SEQ ID NO: 24;
the VH domain comprises a CDR-H1 comprising the amino acid sequence of SEQ ID NO: 25, a CDR-H2 comprising the amino acid sequence of SEQ ID NO: 26, and a CDR-H3 comprising the amino acid sequence of SEQ ID NO: 27; and wherein the VL domain comprises a CDR-L1 comprising the amino acid sequence of SEQ ID NO: 28, a CDR-L2 comprising the amino acid sequence of SEQ ID NO: 29, and a CDR-L3 comprising the amino acid sequence of SEQ ID NO: 30;
the VH domain comprises a CDR-H1 comprising the amino acid sequence of SEQ ID NO: 31, a CDR-H2 comprising the amino acid sequence of SEQ ID NO: 32, and a CDR-H3 comprising the amino acid sequence of SEQ ID NO: 33; and wherein the VL domain comprises a CDR-L1 comprising the amino acid sequence of SEQ ID NO: 34, a CDR-L2 comprising the amino acid sequence of SEQ ID NO: 35, and a CDR-L3 comprising the amino acid sequence of SEQ ID NO: 36;
the VH domain comprises a CDR-H1 comprising the amino acid sequence of SEQ ID NO: 37, a CDR-H2 comprising the amino acid sequence of SEQ ID NO: 38, and a CDR-H3 comprising the amino acid sequence of SEQ ID NO: 39; and wherein the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
the VH domain comprises a CDR-H1 comprising the amino acid sequence of SEQ ID NO: 43, a CDR-H2 comprising the amino acid sequence of SEQ ID NO: 44, and a CDR-H3 comprising the amino acid sequence of SEQ ID NO: 45; and wherein the VL domain comprises a CDR-L1 comprising the amino acid sequence of SEQ ID NO: 46, a CDR-L2 comprising the amino acid sequence of SEQ ID NO: 47, and a CDR-L3 comprising the amino acid sequence of SEQ ID NO: 48;
the VH domain comprises a CDR-H1 comprising the amino acid sequence of SEQ ID NO: 1, a CDR-H2 comprising the amino acid sequence of SEQ ID NO: 2, and a CDR-H3 comprising the amino acid sequence of SEQ ID NO: 3; and wherein the VL domain comprises a CDR-L1 comprising the amino acid sequence of SEQ ID NO: 4, a CDR-L2 comprising the amino acid sequence of SEQ ID NO: 5, and a CDR-L3 comprising the amino acid sequence of SEQ ID NO: 6;
the VH domain comprises a CDR-H1 comprising the amino acid sequence of SEQ ID NO: 177, a CDR-H2 comprising the amino acid sequence of SEQ ID NO: 178, and a CDR-H3 comprising the amino acid sequence of SEQ ID NO: 179; and wherein the VL domain comprises a CDR-L1 comprising the amino acid sequence of SEQ ID NO: 180, a CDR-L2 comprising the amino acid sequence of SEQ ID NO: 181, and a CDR-L3 comprising the amino acid sequence of SEQ ID NO: 182;
the VH domain comprises a CDR-H1 comprising the amino acid sequence of SEQ ID NO: 225, a CDR-H2 comprising the amino acid sequence of SEQ ID NO: 226, and a CDR-H3 comprising the amino acid sequence of SEQ ID NO: 227; and wherein the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228;
the VH domain comprises a CDR-H1 comprising the amino acid sequence of SEQ ID NO: 229, a CDR-H2 comprising the amino acid sequence of SEQ ID NO: 230, and a CDR-H3 comprising the amino acid sequence of SEQ ID NO: 231; and wherein the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
the VH domain comprises a CDR-H1 comprising the amino acid sequence of SEQ ID NO: 225, a CDR-H2 comprising the amino acid sequence of SEQ ID NO: 232, and a CDR-H3 comprising the amino acid sequence of SEQ ID NO: 233; and wherein the VL domain comprises a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236;
the VH domain comprises a CDR-H1 comprising the amino acid sequence of SEQ ID NO: 225, a CDR-H2 comprising the amino acid sequence of SEQ ID NO: 232, and a CDR-H3 comprising the amino acid sequence of SEQ ID NO: 233; and wherein the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228; or the VH domain comprises a CDR-H1 comprising the amino acid sequence of SEQ ID NO: 229, a CDR-H2 comprising the amino acid sequence of SEQ ID NO: 237, and a CDR-H3 comprising the amino acid sequence of SEQ ID NO: 231; and wherein the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42.

In some aspects, the antibody or antigen-binding fragment thereof of the IL-21 and IL-2 fusion proteins comprises a heavy chain variable (VH) domain and a light chain variable (VL) domain, and wherein:
the VH domain comprises a CDR-H1 comprising the amino acid sequence of SEQ ID NO: 51, a CDR-H2 comprising the amino acid sequence of SEQ ID NO: 52, and a CDR-H3 comprising the amino acid sequence of SEQ ID NO: 15; and wherein the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 18;
the VH domain comprises a CDR-H1 comprising the amino acid sequence of SEQ ID NO: 53, a CDR-H2 comprising the amino acid sequence of SEQ ID NO: 52, and a CDR-H3 comprising the amino acid sequence of SEQ ID NO: 21; and wherein the VL domain comprises a CDR-L1 comprising the amino acid sequence of SEQ ID NO: 22, a CDR-L2 comprising the amino acid sequence of SEQ ID NO: 23, and a CDR-L3 comprising the amino acid sequence of SEQ ID NO: 24;
the VH domain comprises a CDR-H1 comprising the amino acid sequence of SEQ ID NO: 49, a CDR-H2 comprising the amino acid sequence of SEQ ID NO: 52, and a CDR-H3 comprising the amino acid sequence of SEQ ID NO: 27; and wherein the VL domain comprises a CDR-L1 comprising the amino acid sequence of SEQ ID NO: 28, a CDR-L2 comprising the amino acid sequence of SEQ ID NO: 29, and a CDR-L3 comprising the amino acid sequence of SEQ ID NO: 30;
the VH domain comprises a CDR-H1 comprising the amino acid sequence of SEQ ID NO: 54, a CDR-H2 comprising the amino acid sequence of SEQ ID NO: 52, and a CDR-H3 comprising the amino acid sequence of SEQ ID NO: 33; and wherein the VL domain comprises a CDR-L1 comprising the amino acid sequence of SEQ ID NO: 34, a CDR-L2 comprising the amino acid sequence of SEQ ID NO: 35, and a CDR-L3 comprising the amino acid sequence of SEQ ID NO: 36;
the VH domain comprises a CDR-H1 comprising the amino acid sequence of SEQ ID NO: 55, a CDR-H2 comprising the amino acid sequence of SEQ ID NO: 56, and a CDR-H3 comprising the amino acid sequence of SEQ ID NO: 39; and wherein the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
the VH domain comprises a CDR-H1 comprising the amino acid sequence of SEQ ID NO: 55, a CDR-H2 comprising the amino acid sequence of SEQ ID NO: 57, and a CDR-H3 comprising the amino acid sequence of SEQ ID NO: 45; and wherein the VL domain comprises a CDR-L1 comprising the amino acid sequence of SEQ ID NO: 46, a CDR-L2 comprising the amino acid sequence of SEQ ID NO: 47, and a CDR-L3 comprising the amino acid sequence of SEQ ID NO: 48;
the VH domain comprises a CDR-H1 comprising the amino acid sequence of SEQ ID NO: 49, a CDR-H2 comprising the amino acid sequence of SEQ ID NO: 50, and a CDR-H3 comprising the amino acid sequence of SEQ ID NO: 3; and wherein the VL domain comprises a CDR-L1 comprising the amino acid sequence of SEQ ID NO: 4, a CDR-L2 comprising the amino acid sequence of SEQ ID NO: 5, and a CDR-L3 comprising the amino acid sequence of SEQ ID NO: 6;
the VH domain comprises a CDR-H1 comprising the amino acid sequence of SEQ ID NO: 183, a CDR-H2 comprising the amino acid sequence of SEQ ID NO: 184, and a CDR-H3 comprising the amino acid sequence of SEQ ID NO: 179; and wherein the VL domain comprises a CDR-L1 comprising the amino acid sequence of SEQ ID NO: 180, a CDR-L2 comprising the amino acid sequence of SEQ ID NO: 181, and a CDR-L3 comprising the amino acid sequence of SEQ ID NO: 182;
the VH domain comprises a CDR-H1 comprising the amino acid sequence of SEQ ID NO: 238, a CDR-H2 comprising the amino acid sequence of SEQ ID NO: 239, and a CDR-H3 comprising the amino acid sequence of SEQ ID NO: 233; and wherein the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228;
the VH domain comprises a CDR-H1 comprising the amino acid sequence of SEQ ID NO: 240, a CDR-H2 comprising the amino acid sequence of SEQ ID NO: 241, and a CDR-H3 comprising the amino acid sequence of SEQ ID NO: 242; and wherein the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
the VH domain comprises a CDR-H1 comprising the amino acid sequence of SEQ ID NO: 238, a CDR-H2 comprising the amino acid sequence of SEQ ID NO: 243, and a CDR-H3 comprising the amino acid sequence of SEQ ID NO: 233; and wherein the VL domain comprises a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236;
the VH domain comprises a CDR-H1 comprising the amino acid sequence of SEQ ID NO: 238, a CDR-H2 comprising the amino acid sequence of SEQ ID NO: 243, and a CDR-H3 comprising the amino acid sequence of SEQ ID NO: 233; and wherein the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228; or
the VH domain comprises a CDR-H1 comprising the amino acid sequence of SEQ ID NO: 240, a CDR-H2 comprising the amino acid sequence of SEQ ID NO: 244, and a CDR-H3 comprising the amino acid sequence of SEQ ID NO: 242; and wherein the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42.

In some aspects, the VH and VL domains comprise amino acid sequences selected from the group:
(a) SEQ ID NOs: 58 and 59, respectively;
(b) SEQ ID NOs: 247 and 248, respectively;
(c) SEQ ID NOs: 62 and 63, respectively;
(d) SEQ ID NOs: 64 and 65, respectively;
(e) SEQ ID NOs: 66 and 67, respectively;
(f) SEQ ID NOs: 68 and 69, respectively;
(g) SEQ ID NOs: 70 and 71, respectively;
(h) SEQ ID NOs: 72 and 73, respectively;
(i) SEQ ID NOs: 185 and 186, respectively;
(j) SEQ ID NOs: 245 and 246, respectively;
(k) SEQ ID NOs: 249 and 250, respectively;
(l) SEQ ID NOs: 251 and 252, respectively;
(m) SEQ ID NOs: 253 and 254, respectively;
(n) SEQ ID NOs: 255 and 256, respectively; and
(o) SEQ ID NOs: 257 and 258, respectively.

In any of the foregoing or related aspects, (i) the IL-2 polypeptide is linked to the antibody or antigen binding fragment directly or by a linker, and/or (ii) the IL-2 polypeptide is linked to the antibody or antigen binding fragment directly or by a linker. In some aspects, (i) the IL-2 polypeptide is linked to the antibody or antigen binding fragment by a Gly-Ser linker, and/or (ii) the IL-2 polypeptide is linked to the antibody or antigen binding fragment by a Gly-Ser linker.

In any of the foregoing or related aspects, the IL-2 fusion protein, the IL-21 fusion protein, or both the IL-2 and IL-21 fusion proteins each comprise:
two antibody heavy chain polypeptides comprising a structure according to formula [I], from N-terminus to C-terminus:

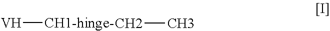

and two antibody light chain polypeptides comprising a structure according to formula [II], from N-terminus to C-terminus:

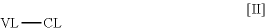

wherein VH is the VH domain, wherein CH1 is an antibody CH1 domain, wherein hinge is an antibody hinge domain, wherein CH2-CH3 is an antibody Fc domain, wherein VL is the VL domain, and wherein CL is an antibody constant light chain domain; and
wherein the N-terminus of the IL-2 polypeptide or the IL-21 polypeptide is fused to the C-terminus of one of the two CH3 domains.

In some aspects, one or both of the antibody heavy chain polypeptides comprise(s) the following amino acid substitutions: L234A, L235A, and G237A, numbering according to EU index. In some aspects, a first of the antibody heavy chain polypeptides comprises amino acid substitutions Y349C and T366W, and a second of the antibody heavy chain polypeptides comprises amino acid substitutions S354C, T366S, L368A and Y407V, numbering according to EU index.

In any of the foregoing or related aspects, the IL-2 polypeptide has a binding affinity to IL-2Rα that is reduced by 50% or more, compared to binding affinity of a human IL-2 polypeptide comprising SEQ ID NO: 81. In some aspects, the IL-2 polypeptide comprises one or more amino acid mutations relative to a human IL-2 polypeptide comprising SEQ ID NO: 81 that reduces the binding affinity of the IL-2 polypeptide. In some aspects, the IL-2 polypeptide comprises the amino acid sequence of SEQ ID NO: 81 with one, two, three, four, or five amino acid substitutions relative to SEQ ID NO: 81, and wherein the one, two, three, four, or five substitution(s) comprise substitution(s) at positions of SEQ ID NO: 81 selected from the group consisting of: Q11, H16, L18, L19, D20, Q22, R38, F42, K43, Y45, E62, P65, E68, V69, L72, D84, S87, N88, V91, 192, T123, Q126, S127, 1129, and S130. In some aspects, the IL-2 polypeptide comprises the sequence of SEQ ID NO: 81 with one of the following sets of amino acid substitutions (relative to the sequence of SEQ ID NO: 81): R38E and F42A; R38D and F42A; F42A and E62Q; R38A and F42K; R38E, F42A, and N88S; R38E, F42A, and N88A; R38E, F42A, and N88G; R38E, F42A, and N88R; R38E, F42A, and N88T; R38E, F42A, and N88D; R38E, F42A, and V91E; R38E, F42A, and D84H; R38E, F42A, and D84K; R38E, F42A, and D84R; H16D, R38E and F42A; H16E, R38E and F42A; R38E, F42A and Q126S; R38D, F42A and N88S; R38D, F42A and N88A; R38D, F42A and N88G; R38D, F42A and N88R; R38D, F42A and N88T; R38D, F42A and N88D; R38D, F42A and V91E; R38D, F42A, and D84H; R38D, F42A, and D84K; R38D, F42A, and D84R; H16D, R38D and F42A; H16E, R38D and F42A; R38D, F42A and Q126S; R38A, F42K, and N88S; R38A, F42K, and N88A; R38A, F42K, and N88G; R38A, F42K, and N88R; R38A, F42K, and N88T; R38A, F42K, and N88D; R38A, F42K, and V91E; R38A, F42K, and D84H; R38A, F42K, and D84K; R38A, F42K, and D84R; H16D, R38A, and F42K; H16E, R38A, and F42K; R38A, F42K, and Q126S; F42A, E62Q, and N88S; F42A, E62Q, and N88A; F42A, E62Q, and N88G; F42A, E62Q, and N88R; F42A, E62Q, and N88T; F42A, E62Q, and N88D; F42A, E62Q, and V91E; F42A, E62Q, and D84H; F42A, E62Q, and D84K; and F42A, E62Q, and D84R. In some aspects, the IL-2 polypeptide comprises the sequence of SEQ ID NO: 81 with a further amino acid substitution relative to SEQ ID NO: 81 at position C125. In some aspects, the IL-2 polypeptide comprises the sequence of SEQ ID NO: 81 with one of the following sets of amino acid substitutions (relative to the sequence of SEQ ID NO: 81): R38E, F42A, and C125A; R38D, F42A, and C125A; F42A, E62Q, and C125A; R38A, F42K, and C125A; R38E, F42A, N88S, and C125A; R38E, F42A, N88A, and C125A; R38E, F42A, N88G, and C125A; R38E, F42A, N88R, and C125A; R38E, F42A, N88D, and C125A; R38E, F42A, N88T, and C125A; R38E, F42A, V91E, and C125A; R38E, F42A, D84H, and C125A; R38E, F42A, D84K, and C125A; R38E, F42A, D84R, and C125A; H16D, R38E, F42A, and C125A; H16E, R38E, F42A, and C125A; R38E, F42A, C125A and Q126S; R38D, F42A, N88S, and C125A; R38D, F42A, N88A, and C125A; R38D, F42A, N88G, and C125A; R38D, F42A, N88R, and C125A; R38D, F42A, N88T, and C125A; R38D, F42A, N88D, and C125A; R38D, F42A, V91E, and C125A; R38D, F42A, D84H, and C125A; R38D, F42A, D84K, and C125A; R38D, F42A, D84R, and C125A; H16D, R38D, F42A, and C125A; H16E, R38D, F42A, and C125A; R38D, F42A, C125A, and Q126S; R38A, F42K, N88S, and C125A; R38A, F42K, N88G, and C125A; R38A, F42K, N88R, and C125A; R38A, F42K, N88T, and C125A; R38A, F42K, N88D, and C125A; R38A, F42K, N88A, and C125A; R38A, F42K, V91E, and C125A; R38A, F42K, D84H, and C125A; R38A, F42K, D84K, and C125A; R38A, F42K, D84R, and C125A; H16D, R38A, F42K, and C125A; H16E, R38A, F42K, and C125A; R38A, F42K, C125A and Q126S; F42A, E62Q, N88S, and C125A; F42A, E62Q, N88A, and C125A; F42A, E62Q, N88G, and C125A; F42A, E62Q, N88R, and C125A; F42A, E62Q, N88T, and C125A; F42A, E62Q, N88D, and C125A; F42A, E62Q, V91E, and C125A; F42A, E62Q, and D84H, and C125A; F42A, E62Q, and D84K, and C125A; F42A, E62Q, and D84R, and C125A; H16D, F42A, and E62Q, and C125A; H16E, F42A, E62Q, and C125A; F42A, E62Q, C125A and Q126S; F42A, N88S, and C125A; F42A, N88A, and C125A; F42A, N88G, and C125A; F42A, N88R, and C125A; F42A, N88T, and C125A; F42A, N88D, and C125A; F42A, V91E, and C125A; F42A, D84H, and C125A; F42A, D84K, and C125A; F42A, D84R, and C125A; H16D, F42A, and C125A; H16E, F42A, and C125A; and F42A, C125A and Q126S. In some aspects, the IL-2 polypeptide comprises the amino acid sequence of SEQ ID NO: 80 or SEQ ID NO: 297. In some aspects, the IL-2 polypeptide comprises a sequence selected from the group consisting of SEQ ID NOs: 80, 85-155, 190-216, 297, and 354-383.

In any of the foregoing or related aspects, the IL-21 polypeptide comprises an amino acid sequence that is at least 80% identical to a human IL-21 polypeptide comprising SEQ ID NO: 390. In some aspects, the IL-21 polypeptide has an isoelectric point that is at least about 0.6 units to about 5 units lower, compared to that of the human IL-21 polypeptide. In some aspects, the IL-21 polypeptide comprises at least one amino acid substitution that reduces the isoelectric point of the IL-21 polypeptide by about 0.6 units to about 5 units relative to the human IL-21 polypeptide without the amino acid substitution. In some aspects, the IL-21 polypeptide comprises at least four amino acid substitutions that reduce the isoelectric point of the IL-21 polypeptide about 0.6 units to about 5 units relative to the human IL-21 polypeptide without the amino acid substitutions. In some aspects, the IL-21 polypeptide comprises up to 10, 11, 12, 13, 14, 15, 16, 17, 18, 19 or 20 amino acid substitutions that reduce the isoelectric point. In some aspects, the human IL-21 polypeptide has an isoelectric point of about 9.42. In some aspects, the IL-21 polypeptide has an isoelectric point of about 7.12 to about 8.72. In some aspects, the IL-21 polypeptide results in an improved exposure following administration to the subject relative to the human IL-21 polypeptide, as measured by at least about 1.5 times greater area under the curve (AUC) for the IL-21 polypeptide. In some aspects, the human IL-21 polypeptide comprises a region of about 2 to 20 positively charged amino acid residues, and wherein the IL-21 polypeptide comprises at least one amino acid substitution of one or more of the positively charged amino acid residues. In some aspects, the region does not include amino acid residues that bind an IL-21 receptor. In some aspects, the region comprises amino acid residues S80 to T92 of the human IL-21 polypeptide comprising SEQ ID NO: 390. In some aspects, the human IL-21 polypeptide comprises at least one positively charged amino acid residue on the surface of the human IL-21 polypeptide in a three-dimensional structure of the human IL-21 polypeptide and does not bind an IL-21 receptor, and wherein the IL-21 polypeptide comprises at least one amino acid substitution of at the least one positively charged amino acid residue. In some aspects, the IL-21 polypeptide does not comprise an amino acid substitution at G84 of the human IL-21 polypeptide comprising SEQ ID NO: 390. In some aspects, the IL-21 polypeptide comprises 4, 5, 6, 7, 8, 9, 10, 11, or 12 amino acid substitutions at positions selected from S80, T81, N82, A83, R85, R86, Q87, K88, H89, R90, L91, or T92 of SEQ ID NO: 390. In some aspects, the IL-21 polypeptide comprises 4, 5, 6, 7, 8, 9, 10, 11, or 12 amino acid substitutions selected from S80G, T81G, N82G, N82E, A83G, A83E, A83S, R85G, R85E, R85S, R86G, R86E, Q87G, Q87E, Q87S, K88G, H89G, H89S, R90G, R90S, R90E, R90A, L91G, L91S, T92G, T92S. In some aspects, the IL-21 polypeptide comprises (a) R85G, R86G, K88G and R90E, or (b) S80G, T81G, N82E, A83G, R85G, R86G, Q87G, K88G, H89G, R90E, L91G, and T92G. In some aspects, the IL-21 polypeptide comprises the amino acid sequence: QGQDRHMIRMRQLIDIVDQLKNYVNDLVPEFLPAPEDVETNCEWSAFSCFQKAQLK SANTGNNERIINVSIKKLKRKPPX1X₂X₃X₄GX5X₆X₇X₈X₉X₁₀X₁₁X₁₂CPSCDSYEKKPPK EFLERFKSLLQKMIHQHLSSRTHGSEDS (SEQ ID NO: 554), wherein X₁=G, S; X₂=G, T; X₃=G, E, N; X₄=G, S, E, A; X₅=G, E, S, R; X₆=G, E, R; X₇=S, G, E, Q; X₈=G, K; X₉=G, S, H; X₁₀=A, E, S, G, R; X₁₁=S, G, L; and X₁₂=G, S, T, provided at least one of X₅, X₆, X₈, X₉, and X₁₀ is not the amino acid residue at the identical position set forth in SEQ ID NO: 390, optionally wherein:

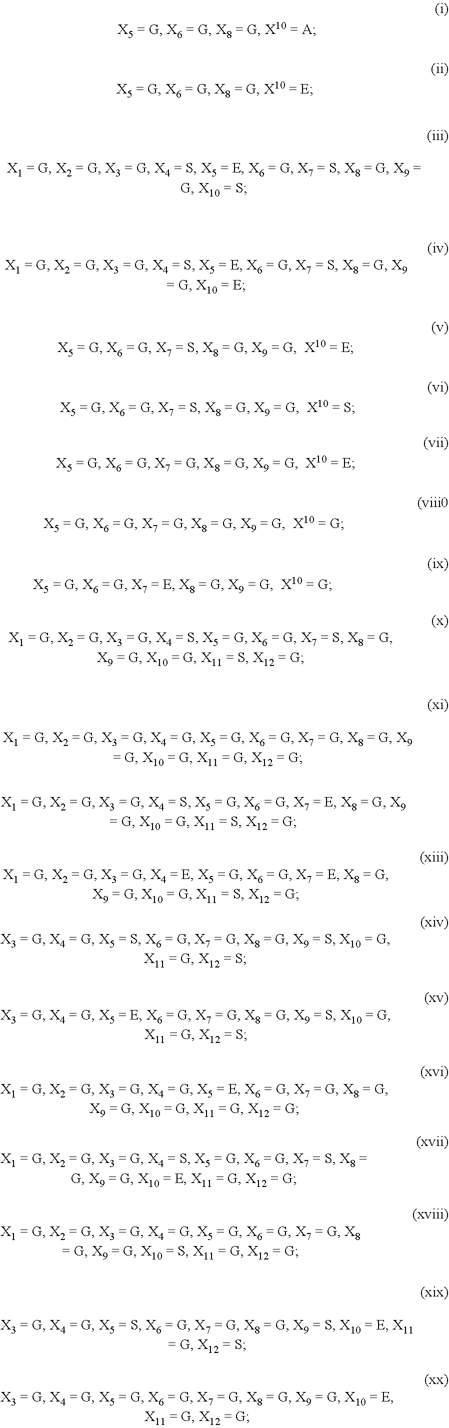

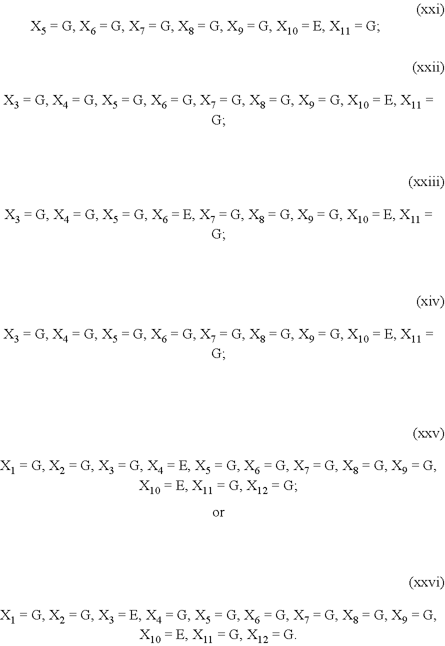

In some aspects, the IL-12 polypeptide comprises an amino acid sequence at least 90% identical to an amino acid sequence selected from SEQ ID NOs: 391-421. In some aspects, the IL-21 polypeptide comprises an amino acid sequence selected from SEQ ID NOs: 391-421. In some aspects, the IL-21 polypeptide comprises the amino acid sequence of SEQ ID NO: 421. In some aspects, the IL-21 polypeptide comprises at least one amino acid substitution that reduces binding to an IL-21 receptor compared to binding by the human IL-21 polypeptide. In some aspects, the at least one amino acid substitution is at one or more amino acid residues at positions R5, I8, R9, R11, L13, I14, I16, V17, D18, K72, K73, L74, K75, R76, K77, or K117, of SEQ ID NO: 390. In some aspects, the at least one amino acid substitution is selected from: R5F, R5A, R5E, R5S, R5T, R5N, R5Q, R5V, R5I, R5L, R5Y, 18E, R9A, R9D, R9E, R9H, R9S, R9T, R9N, R9G, R9V, R9I, R9L, R9Y, RI1D, RI1E, L13F, L13R, 114D, 116A, 116S, 116R, V171, V17A, D18A, K72A, K72E, K73A, K73E, K75A, K75E, L74I, L74F, L74M, L74V, R76E, R76F, R76A, R76N, R76D, R76S, R76T, R76Q, R76V, R76I, R76L, R76Y, R76M, K77A, K77E, and K117A. In some aspects, the at least one amino acid substitution is R76E or R76Q. In some aspects, the IL-21 polypeptide comprises an amino acid sequence at least 90% identical to an amino acid sequence selected from SEQ ID NOs: 422-491. In some aspects, the IL-21 polypeptide comprises an amino acid sequence selected from SEQ ID NOs: 422-491.

In any of the foregoing or related aspects, the IL-2 fusion protein comprises:
one or two light chains comprising the amino acid sequence of SEQ ID NO: 156, a heavy chain comprising the amino acid sequence of SEQ ID NO: 157, and a heavy chain comprising the amino acid sequence of SEQ ID NO: 158;
one or two light chains comprising the amino acid sequence of SEQ ID NO: 159, a heavy chain comprising the amino acid sequence of SEQ ID NO: 160, and a heavy chain comprising the amino acid sequence of SEQ ID NO: 161;
one or two light chains comprising the amino acid sequence of SEQ ID NO: 162, a heavy chain comprising the amino acid sequence of SEQ ID NO: 163, and a heavy chain comprising the amino acid sequence of SEQ ID NO: 164;
one or two light chains comprising the amino acid sequence of SEQ ID NO: 165, a heavy chain comprising the amino acid sequence of SEQ ID NO: 166, and a heavy chain comprising the amino acid sequence of SEQ ID NO: 167.
one or two light chains comprising the amino acid sequence of SEQ ID NO: 168, a heavy chain comprising the amino acid sequence of SEQ ID NO: 169, and a heavy chain comprising the amino acid sequence of SEQ ID NO: 170;
one or two light chains comprising the amino acid sequence of SEQ ID NO: 171, a heavy chain comprising the amino acid sequence of SEQ ID NO: 172, and a heavy chain comprising the amino acid sequence of SEQ ID NO: 173;
one or two light chains comprising the amino acid sequence of SEQ ID NO: 174, a heavy chain comprising the amino acid sequence of SEQ ID NO: 175, and a heavy chain comprising the amino acid sequence of SEQ ID NO: 176;
one or two light chains comprising the amino acid sequence of SEQ ID NO: 187, a heavy chain comprising the amino acid sequence of SEQ ID NO: 188, and a heavy chain comprising the amino acid sequence of SEQ ID NO: 189;
one or two light chains comprising the amino acid sequence of SEQ ID NO: 298, a heavy chain comprising the amino acid sequence of SEQ ID NO: 299, and a heavy chain comprising the amino acid sequence of SEQ ID NO: 300;
one or two light chains comprising the amino acid sequence of SEQ ID NO: 302, a heavy chain comprising the amino acid sequence of SEQ ID NO: 303, and a heavy chain comprising the amino acid sequence of SEQ ID NO: 304;
one or two light chains comprising the amino acid sequence of SEQ ID NO: 306, a heavy chain comprising the amino acid sequence of SEQ ID NO: 307, and a heavy chain comprising the amino acid sequence of SEQ ID NO: 308;
one or two light chains comprising the amino acid sequence of SEQ ID NO: 310, a heavy chain comprising the amino acid sequence of SEQ ID NO: 311, and a heavy chain comprising the amino acid sequence of SEQ ID NO: 312;
one or two light chains comprising the amino acid sequence of SEQ ID NO: 314, a heavy chain comprising the amino acid sequence of SEQ ID NO: 315, and a heavy chain comprising the amino acid sequence of SEQ ID NO: 316;
one or two light chains comprising the amino acid sequence of SEQ ID NO: 318, a heavy chain comprising the amino acid sequence of SEQ ID NO: 319, and a heavy chain comprising the amino acid sequence of SEQ ID NO: 320;
one or two light chains comprising the amino acid sequence of SEQ ID NO: 322, a heavy chain comprising the amino acid sequence of SEQ ID NO: 323, and a heavy chain comprising the amino acid sequence of SEQ ID NO: 324;
one or two light chains comprising the amino acid sequence of SEQ ID NO: 326, a heavy chain comprising the amino acid sequence of SEQ ID NO: 327, and a heavy chain comprising the amino acid sequence of SEQ ID NO: 328;
one or two light chains comprising the amino acid sequence of SEQ ID NO: 330, a heavy chain comprising the amino acid sequence of SEQ ID NO: 331, and a heavy chain comprising the amino acid sequence of SEQ ID NO: 332;
one or two light chains comprising the amino acid sequence of SEQ ID NO: 334, a heavy chain comprising the amino acid sequence of SEQ ID NO: 335, and a heavy chain comprising the amino acid sequence of SEQ ID NO: 336;
one or two light chains comprising the amino acid sequence of SEQ ID NO: 338, a heavy chain comprising the amino acid sequence of SEQ ID NO: 339, and a heavy chain comprising the amino acid sequence of SEQ ID NO: 340;
one or two light chains comprising the amino acid sequence of SEQ ID NO: 342, a heavy chain comprising the amino acid sequence of SEQ ID NO: 343, and a heavy chain comprising the amino acid sequence of SEQ ID NO: 344;
one or two light chains comprising the amino acid sequence of SEQ ID NO: 346, a heavy chain comprising the amino acid sequence of SEQ ID NO: 347, and a heavy chain comprising the amino acid sequence of SEQ ID NO: 348;
one or two light chains comprising the amino acid sequence of SEQ ID NO: 350, a heavy chain comprising the amino acid sequence of SEQ ID NO: 351, and a heavy chain comprising the amino acid sequence of SEQ ID NO: 352;
one or two light chains comprising the amino acid sequence of SEQ ID NO: 156, a heavy chain comprising the amino acid sequence of SEQ ID NO: 157, and a heavy chain comprising the amino acid sequence of SEQ ID NO: 217;
one or two light chains comprising the amino acid sequence of SEQ ID NO: 159, a heavy chain comprising the amino acid sequence of SEQ ID NO: 160, and a heavy chain comprising the amino acid sequence of SEQ ID NO: 218;
one or two light chains comprising the amino acid sequence of SEQ ID NO: 162, a heavy chain comprising the amino acid sequence of SEQ ID NO: 163, and a heavy chain comprising the amino acid sequence of SEQ ID NO: 219;
one or two light chains comprising the amino acid sequence of SEQ ID NO: 165, a heavy chain comprising the amino acid sequence of SEQ ID NO: 166, and a heavy chain comprising the amino acid sequence of SEQ ID NO: 220;
one or two light chains comprising the amino acid sequence of SEQ ID NO: 168, a heavy chain comprising the amino acid sequence of SEQ ID NO: 169, and a heavy chain comprising the amino acid sequence of SEQ ID NO: 221;
one or two light chains comprising the amino acid sequence of SEQ ID NO: 171, a heavy chain comprising the amino acid sequence of SEQ ID NO: 172, and a heavy chain comprising the amino acid sequence of SEQ ID NO: 222;
one or two light chains comprising the amino acid sequence of SEQ ID NO: 174, a heavy chain comprising the amino acid sequence of SEQ ID NO: 175, and a heavy chain comprising the amino acid sequence of SEQ ID NO: 223;
one or two light chains comprising the amino acid sequence of SEQ ID NO: 187, a heavy chain comprising the amino acid sequence of SEQ ID NO: 188, and a heavy chain comprising the amino acid sequence of SEQ ID NO: 224;
one or two light chains comprising the amino acid sequence of SEQ ID NO: 298, a heavy chain comprising the amino acid sequence of SEQ ID NO: 299, and a heavy chain comprising the amino acid sequence of SEQ ID NO: 301;
one or two light chains comprising the amino acid sequence of SEQ ID NO: 302, a heavy chain comprising the amino acid sequence of SEQ ID NO: 303, and a heavy chain comprising the amino acid sequence of SEQ ID NO: 305;
one or two light chains comprising the amino acid sequence of SEQ ID NO: 306, a heavy chain comprising the amino acid sequence of SEQ ID NO: 307, and a heavy chain comprising the amino acid sequence of SEQ ID NO: 309;
one or two light chains comprising the amino acid sequence of SEQ ID NO: 310, a heavy chain comprising the amino acid sequence of SEQ ID NO: 311, and a heavy chain comprising the amino acid sequence of SEQ ID NO: 313;
one or two light chains comprising the amino acid sequence of SEQ ID NO: 314, a heavy chain comprising the amino acid sequence of SEQ ID NO: 315, and a heavy chain comprising the amino acid sequence of SEQ ID NO: 317;
one or two light chains comprising the amino acid sequence of SEQ ID NO: 318, a heavy chain comprising the amino acid sequence of SEQ ID NO: 319, and a heavy chain comprising the amino acid sequence of SEQ ID NO: 321;
one or two light chains comprising the amino acid sequence of SEQ ID NO: 322, a heavy chain comprising the amino acid sequence of SEQ ID NO: 323, and a heavy chain comprising the amino acid sequence of SEQ ID NO: 325;
one or two light chains comprising the amino acid sequence of SEQ ID NO: 326, a heavy chain comprising the amino acid sequence of SEQ ID NO: 327, and a heavy chain comprising the amino acid sequence of SEQ ID NO: 329;
one or two light chains comprising the amino acid sequence of SEQ ID NO: 330, a heavy chain comprising the amino acid sequence of SEQ ID NO: 331, and a heavy chain comprising the amino acid sequence of SEQ ID NO: 333;
one or two light chains comprising the amino acid sequence of SEQ ID NO: 334, a heavy chain comprising the amino acid sequence of SEQ ID NO: 335, and a heavy chain comprising the amino acid sequence of SEQ ID NO: 337;
one or two light chains comprising the amino acid sequence of SEQ ID NO: 338, a heavy chain comprising the amino acid sequence of SEQ ID NO: 339, and a heavy chain comprising the amino acid sequence of SEQ ID NO: 341;
one or two light chains comprising the amino acid sequence of SEQ ID NO: 342, a heavy chain comprising the amino acid sequence of SEQ ID NO: 343, and a heavy chain comprising the amino acid sequence of SEQ ID NO: 345;
one or two light chains comprising the amino acid sequence of SEQ ID NO: 346, a heavy chain comprising the amino acid sequence of SEQ ID NO: 347, and a heavy chain comprising the amino acid sequence of SEQ ID NO: 349; or
one or two light chains comprising the amino acid sequence of SEQ ID NO: 350, a heavy chain comprising the amino acid sequence of SEQ ID NO: 351, and a heavy chain comprising the amino acid sequence of SEQ ID NO: 353.

In any of the foregoing or related aspects, the IL-2 fusion protein comprises four polypeptide chains, wherein:
the first polypeptide chain comprises the amino acid sequence of SEQ ID NO: 334, the second polypeptide chain comprises the amino acid sequence of SEQ ID NO: 335, the third polypeptide chain comprises the amino acid sequence of SEQ ID NO: 336, and the fourth polypeptide chain comprises the amino acid sequence of SEQ ID NO: 334;
the first polypeptide chain comprises the amino acid sequence of SEQ ID NO: 334, the second polypeptide chain comprises the amino acid sequence of SEQ ID NO: 335, the third polypeptide chain comprises the amino acid sequence of SEQ ID NO: 337, and the fourth polypeptide chain comprises the amino acid sequence of SEQ ID NO: 334;
the first polypeptide chain comprises the amino acid sequence of SEQ ID NO: 338, the second polypeptide chain comprises the amino acid sequence of SEQ ID NO: 339, the third polypeptide chain comprises the amino acid sequence of SEQ ID NO: 340, and the fourth polypeptide chain comprises the amino acid sequence of SEQ ID NO: 338; or
the first polypeptide chain comprises the amino acid sequence of SEQ ID NO: 338, the second polypeptide chain comprises the amino acid sequence of SEQ ID NO: 339, the third polypeptide chain comprises the amino acid sequence of SEQ ID NO: 341, and the fourth polypeptide chain comprises the amino acid sequence of SEQ ID NO: 338.

In any of the foregoing or related aspects, the IL-21 fusion protein comprises:
one or two light chains comprising the amino acid sequence of SEQ ID NO: 557, a heavy chain comprising the amino acid sequence of SEQ ID NO: 558, and a heavy chain comprising the amino acid sequence of SEQ ID NO: 559;
one or two light chains comprising the amino acid sequence of SEQ ID NO: 561, a heavy chain comprising the amino acid sequence of SEQ ID NO: 562, and a heavy chain comprising the amino acid sequence of SEQ ID NO: 563;
one or two light chains comprising the amino acid sequence of SEQ ID NO: 565, a heavy chain comprising the amino acid sequence of SEQ ID NO: 566, and a heavy chain comprising the amino acid sequence of SEQ ID NO: 567;
one or two light chains comprising the amino acid sequence of SEQ ID NO: 569, a heavy chain comprising the amino acid sequence of SEQ ID NO: 570, and a heavy chain comprising the amino acid sequence of SEQ ID NO: 571;
one or two light chains comprising the amino acid sequence of SEQ ID NO: 573, a heavy chain comprising the amino acid sequence of SEQ ID NO: 574, and a heavy chain comprising the amino acid sequence of SEQ ID NO: 575;
one or two light chains comprising the amino acid sequence of SEQ ID NO: 577, a heavy chain comprising the amino acid sequence of SEQ ID NO: 578, and a heavy chain comprising the amino acid sequence of SEQ ID NO: 579;
one or two light chains comprising the amino acid sequence of SEQ ID NO: 581, a heavy chain comprising the amino acid sequence of SEQ ID NO: 582, and a heavy chain comprising the amino acid sequence of SEQ ID NO: 583;
one or two light chains comprising the amino acid sequence of SEQ ID NO: 585, a heavy chain comprising the amino acid sequence of SEQ ID NO: 586, and a heavy chain comprising the amino acid sequence of SEQ ID NO: 587;
one or two light chains comprising the amino acid sequence of SEQ ID NO: 589, a heavy chain comprising the amino acid sequence of SEQ ID NO: 590, and a heavy chain comprising the amino acid sequence of SEQ ID NO: 591;
one or two light chains comprising the amino acid sequence of SEQ ID NO: 557, a heavy chain comprising the amino acid sequence of SEQ ID NO: 558, and a heavy chain comprising the amino acid sequence of SEQ ID NO: 560;
one or two light chains comprising the amino acid sequence of SEQ ID NO: 561, a heavy chain comprising the amino acid sequence of SEQ ID NO: 562, and a heavy chain comprising the amino acid sequence of SEQ ID NO: 564;
one or two light chains comprising the amino acid sequence of SEQ ID NO: 565, a heavy chain comprising the amino acid sequence of SEQ ID NO: 566, and a heavy chain comprising the amino acid sequence of SEQ ID NO: 568;
one or two light chains comprising the amino acid sequence of SEQ ID NO: 569, a heavy chain comprising the amino acid sequence of SEQ ID NO: 570, and a heavy chain comprising the amino acid sequence of SEQ ID NO: 572;
one or two light chains comprising the amino acid sequence of SEQ ID NO: 573, a heavy chain comprising the amino acid sequence of SEQ ID NO: 574, and a heavy chain comprising the amino acid sequence of SEQ ID NO: 576;
one or two light chains comprising the amino acid sequence of SEQ ID NO: 577, a heavy chain comprising the amino acid sequence of SEQ ID NO: 578, and a heavy chain comprising the amino acid sequence of SEQ ID NO: 580;
one or two light chains comprising the amino acid sequence of SEQ ID NO: 581, a heavy chain comprising the amino acid sequence of SEQ ID NO: 582, and a heavy chain comprising the amino acid sequence of SEQ ID NO: 584;
one or two light chains comprising the amino acid sequence of SEQ ID NO: 585, a heavy chain comprising the amino acid sequence of SEQ ID NO: 586, and a heavy chain comprising the amino acid sequence of SEQ ID NO: 588; or
one or two light chains comprising the amino acid sequence of SEQ ID NO: 589, a heavy chain comprising the amino acid sequence of SEQ ID NO: 590, and a heavy chain comprising the amino acid sequence of SEQ ID NO: 592.

In any of the foregoing or related aspects, the IL-21 fusion protein comprises four polypeptide chains, wherein:
the first polypeptide chain comprises the amino acid sequence of SEQ ID NO: 557, the second polypeptide chain comprises the amino acid sequence of SEQ ID NO: 558, the third polypeptide chain comprises the amino acid sequence of SEQ ID NO: 559, and the fourth polypeptide chain comprises the amino acid sequence of SEQ ID NO: 557;
the first polypeptide chain comprises the amino acid sequence of SEQ ID NO: 557, the second polypeptide chain comprises the amino acid sequence of SEQ ID NO: 558, the third polypeptide chain comprises the amino acid sequence of SEQ ID NO: 560, and the fourth polypeptide chain comprises the amino acid sequence of SEQ ID NO: 557;
the first polypeptide chain comprises the amino acid sequence of SEQ ID NO: 561, the second polypeptide chain comprises the amino acid sequence of SEQ ID NO: 562, the third polypeptide chain comprises the amino acid sequence of SEQ ID NO: 563, and the fourth polypeptide chain comprises the amino acid sequence of SEQ ID NO: 561;
the first polypeptide chain comprises the amino acid sequence of SEQ ID NO: 561, the second polypeptide chain comprises the amino acid sequence of SEQ ID NO: 562, the third polypeptide chain comprises the amino acid sequence of SEQ ID NO: 564, and the fourth polypeptide chain comprises the amino acid sequence of SEQ ID NO: 561;
the first polypeptide chain comprises the amino acid sequence of SEQ ID NO: 565, the second polypeptide chain comprises the amino acid sequence of SEQ ID NO: 566, the third polypeptide chain comprises the amino acid sequence of SEQ ID NO: 567, and the fourth polypeptide chain comprises the amino acid sequence of SEQ ID NO: 565;
the first polypeptide chain comprises the amino acid sequence of SEQ ID NO: 565, the second polypeptide chain comprises the amino acid sequence of SEQ ID NO: 566, the third polypeptide chain comprises the amino acid sequence of SEQ ID NO: 568, and the fourth polypeptide chain comprises the amino acid sequence of SEQ ID NO: 565;
the first polypeptide chain comprises the amino acid sequence of SEQ ID NO: 569, the second polypeptide chain comprises the amino acid sequence of SEQ ID NO: 570, the third polypeptide chain comprises the amino acid sequence of SEQ ID NO: 571, and the fourth polypeptide chain comprises the amino acid sequence of SEQ ID NO: 569;
the first polypeptide chain comprises the amino acid sequence of SEQ ID NO: 569, the second polypeptide chain comprises the amino acid sequence of SEQ ID NO: 570, the third polypeptide chain comprises the amino acid sequence of SEQ ID NO: 572, and the fourth polypeptide chain comprises the amino acid sequence of SEQ ID NO: 569;
the first polypeptide chain comprises the amino acid sequence of SEQ ID NO: 573, the second polypeptide chain comprises the amino acid sequence of SEQ ID NO: 574, the third polypeptide chain comprises the amino acid sequence of SEQ ID NO: 575, and the fourth polypeptide chain comprises the amino acid sequence of SEQ ID NO: 573;
the first polypeptide chain comprises the amino acid sequence of SEQ ID NO: 573, the second polypeptide chain comprises the amino acid sequence of SEQ ID NO: 574, the third polypeptide chain comprises the amino acid sequence of SEQ ID NO: 576, and the fourth polypeptide chain comprises the amino acid sequence of SEQ ID NO: 573;
the first polypeptide chain comprises the amino acid sequence of SEQ ID NO: 577, the second polypeptide chain comprises the amino acid sequence of SEQ ID NO: 578, the third polypeptide chain comprises the amino acid sequence of SEQ ID NO: 579, and the fourth polypeptide chain comprises the amino acid sequence of SEQ ID NO: 577;
the first polypeptide chain comprises the amino acid sequence of SEQ ID NO: 577, the second polypeptide chain comprises the amino acid sequence of SEQ ID NO: 578, the third polypeptide chain comprises the amino acid sequence of SEQ ID NO: 580, and the fourth polypeptide chain comprises the amino acid sequence of SEQ ID NO: 577;
the first polypeptide chain comprises the amino acid sequence of SEQ ID NO: 581, the second polypeptide chain comprises the amino acid sequence of SEQ ID NO: 582, the third polypeptide chain comprises the amino acid sequence of SEQ ID NO: 583, and the fourth polypeptide chain comprises the amino acid sequence of SEQ ID NO: 581;
the first polypeptide chain comprises the amino acid sequence of SEQ ID NO: 581, the second polypeptide chain comprises the amino acid sequence of SEQ ID NO: 582, the third polypeptide chain comprises the amino acid sequence of SEQ ID NO: 584, and the fourth polypeptide chain comprises the amino acid sequence of SEQ ID NO: 581;
the first polypeptide chain comprises the amino acid sequence of SEQ ID NO: 585, the second polypeptide chain comprises the amino acid sequence of SEQ ID NO: 586, the third polypeptide chain comprises the amino acid sequence of SEQ ID NO: 587, and the fourth polypeptide chain comprises the amino acid sequence of SEQ ID NO: 585;
the first polypeptide chain comprises the amino acid sequence of SEQ ID NO: 585, the second polypeptide chain comprises the amino acid sequence of SEQ ID NO: 586, the third polypeptide chain comprises the amino acid sequence of SEQ ID NO: 588, and the fourth polypeptide chain comprises the amino acid sequence of SEQ ID NO: 585;
the first polypeptide chain comprises the amino acid sequence of SEQ ID NO: 589, the second polypeptide chain comprises the amino acid sequence of SEQ ID NO: 590, the third polypeptide chain comprises the amino acid sequence of SEQ ID NO: 591, and the fourth polypeptide chain comprises the amino acid sequence of SEQ ID NO: 589; or
the first polypeptide chain comprises the amino acid sequence of SEQ ID NO: 589, the second polypeptide chain comprises the amino acid sequence of SEQ ID NO: 590, the third polypeptide chain comprises the amino acid sequence of SEQ ID NO: 592, and the fourth polypeptide chain comprises the amino acid sequence of SEQ ID NO: 589.

In any of the foregoing or related aspects, the IL-21 fusion protein is administered prior to the administration of the IL-2 fusion protein. In other aspects, the IL-2 fusion protein is administered prior to the administration of the IL-21 fusion protein. In some aspects, the IL-21 fusion protein and IL-2 fusion protein are administered concurrently, sequentially, or simultaneously.

In any of the foregoing or related aspects, the IL-21 fusion protein is formulated in a pharmaceutical composition comprising a pharmaceutically acceptable carrier. In some aspects, the IL-2 fusion protein is formulated in a pharmaceutical composition comprising a pharmaceutically acceptable carrier. In some aspects, the IL-21 fusion protein and the IL-2 fusion protein are formulated in the same pharmaceutical composition. In other aspects, the IL-21 fusion protein and the IL-2 fusion protein are formulated in separate pharmaceutical compositions.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 shows schematics of the xmCD8-mIL21v1.1 and xmCD8.1-mIL2v fusion proteins. Both fusions include an anti-mouse CD8 IgG, and a single copy of the cytokine (mIL21v1.1 or mIL2v) fused to the C-terminus of one of the heavy chains. In some cases, the Fc region for both schematics comprises a knob-in-hole heterodimerization mutations. In some cases, the Fc region for both schematics does not comprise a knob-in-hole heterodimerization mutations.

FIGS. 2A-2D show MC38 efficacy studies, comparing single agent versus combination of xmCD8-mIL21v1.1 and xmCD8.1-mIL2v dosed simultaneously at different dose levels. Single agent versus combinations are shown for 0.3 mg/kg xmCD8.1-mIL2v and 0.1 mg/kg xmCD8-mIL21v1.1 (FIG. 2A), 0.3 mg/kg xmCD8.1-mIL2v and 0.03 mg/kg xmCD8-mIL21v1.1 (FIG. 2B), 0.1 mg/kg xmCD8.1-mIL2v and 0.1 mg/kg xmCD8-mIL21v1.1 (FIG. 2C), 0.1 mg/kg xmCD8.1-mIL2v and 0.03 mg/kg xmCD8-mIL21v1.1 (FIG. 2D).

FIG. 3 shows MC38 efficacy studies, comparing single agent versus combination of xmCD8-mIL21v1.1 and xmCD8.1-mIL2v dosed 1 hour apart at different dose levels. Separate groups for combination treatment are shown with either xmCD8-mIL21v1.1 dosed first or xmCD8.1-mIL2v dosed first. Single agent versus combinations are shown for 0.1 mg/kg xmCD8.1-mIL2v and 0.1 mg/kg xmCD8-mIL21v1.1.

### DETAILED DESCRIPTION

The present disclosure is based, at least in part, on the discovery that a combination of an IL-2 fusion protein and an IL-21 fusion protein, each fusion protein comprising an antibody that specifically binds human CD8, reduces or inhibits tumor growth in vivo greater than administration of either fusion protein alone. Without wishing to be bound by theory, targeting IL-2 and IL-21 to CD8+ T cells with an anti-CD8 specific antibody selectively activates CD8+ T cells to induce an anti-tumor immune response. As IL-2 activates CD8+ T cells via STAT5 phosphorylation and thus enhances the cytotoxic function while promoting proliferation, while IL-21 activates CD8+ T cells via STAT3 phosphorylation and maintains T cell function, prevents exhaustion, promotes memory phenotype and enhances the cytotoxic function, it is believed targeting these two separate complementary non-redundant pathways provides for enhanced CD8+ T cell activation and thus therapeutic efficacy.

The present disclosure describes, inter alia, methods for treating a disease, such as cancer or an infection, in mammalian subjects. In some embodiments, the treatment comprises administering to the subject a combination of fusion proteins that bind to human CD8. In some embodiments, the fusion proteins include IL-21 or IL-2. In some embodiments, the IL-21 fusion proteins and IL-2 fusion proteins comprise human or humanized antibodies, antigen binding fragments, or fusion proteins that bind human CD8.

### Definitions

As used in this specification and the appended claims, the singular forms “a”, “an” and “the” include plural referents unless the content clearly dictates otherwise. Thus, for example, reference to “a molecule” optionally includes a combination of two or more such molecules, and the like.

It is understood that aspects and embodiments of the present disclosure include “comprising,” “consisting,” and “consisting essentially of” aspects and embodiments.

The term “about” as used herein refers to the usual error range for the respective value readily known to the skilled person in this technical field. If there are uses of the term which are not clear to persons of ordinary skill given the context in which it is used, “about” will mean up to plus or minus 10% of the particular value. Reference to “about” a value or parameter herein includes (and describes) embodiments that are directed to that value or parameter per se.

As used herein, “area under the curve” or “AUC” is a pharmacokinetic parameter that refers to the area under the plot of plasma concentration of a drug (e.g., IL-21 polypeptide or fusion protein described herein) versus time after dosage. In some embodiments, the AUC measures a patient's exposure to a drug and depends on dose, bioavailability, and clearance. Methods for determining the AUC are known to those of skill in the art and include, but is not limited to, the Rectangle Method, the Trapezoidal Rule, and Simpson's Rule.

“Immune cells” as used here are cells of the immune system that react to organisms or other entities that are deemed foreign to the immune system of the host. They protect the host against foreign pathogens, organisms and diseases. Immune cells, also called leukocytes, are involved in both innate and adaptive and immune responses to fight pathogens. Innate immune responses occur immediately upon exposure to pathogens without additional priming or learning processes. Adaptive immune processes require initial priming, and subsequently create memory, which in turn leads to enhanced responsiveness during subsequent encounters with the same pathogen. Innate immune cells include, but are not limited to monocytes, macrophages, dendritic cells, innate lymphoid cells (ILCs) including natural killer (NK) cells, neutrophils, megakaryocytes, eosinophils and basophils. Adaptive immune cells include B and T lymphocytes/cells. T cells subsets include, but are not limited to, alpha beta CD4+ T (naïve CD4+, memory CD4+, effector memory CD4+, effector CD4+, regulatory CD4+), and alpha beta CD8+ T (naïve CD8+, memory CD8+, effector memory CD8+, effector CD8+). B cell subsets include, but is not limited to, naïve B, memory B, and plasma cells. NK T cells and T gamma delta (Ty6) cells exhibit properties of both innate and adaptive lymphocytes.

“T cells” or “T lymphocytes” are immune cells that play a key role in the orchestration of immune responses in health and disease. Two major T cell subsets exist that have unique functions and properties: T cells that express the CD8 antigen (CD8⁺ T cells) are cytotoxic or killer T cells that can lyse target cells using the cytotoxic proteins such as granzymes and perforin; and T cells that express the CD4 antigen (CD4⁺ T cells) are helper T cells that are capable of regulating the function of many other immune cell types including that of CD8⁺ T cells, B cells, macrophages etc. Furthermore, CD4⁺ T cells are further subdivided into several subsets such as: T regulatory (Treg) cells that are capable of suppressing the immune response, and T helper 1 (Th1), T helper 2 (Th2), and T helper 17 (Th17) cells that regulate different types of immune responses by secreting immunomodulatory proteins such as cytokines. T cells recognize their targets via alpha beta T cell receptors that bind to unique antigen-specific motifs and this recognition mechanism is generally required in order to trigger their cytotoxic and cytokine-secreting functions. “Innate lymphocytes” can also exhibit properties of CD8⁺ and CD4⁺ T cells, such as the cytotoxic activity or the secretion of Th1, Th2, and Th17 cytokines. Some of these innate lymphocyte subsets include NK cells and ILC1, ILC2, and ILC3 cells; and innate-like T cells such as Ty6 cells; and NK T cells. Typically, these cells can rapidly respond to inflammatory stimuli from infected or injured tissues, such as immunomodulatory cytokines, but unlike alpha beta T cells, they can respond without the need to recognize antigen-specific patterns.

“Cytokine” is a form of immunomodulatory polypeptide that mediates cross-talk between initiating/primary cells and target/effector cells. It can function as a soluble form or cell-surface associated to bind the “cytokine receptor” on target immune cells to activate signaling. “Cytokine receptor” as used here is the polypeptide on the cell surface that activates intracellular signaling upon binding the cytokine on the extracellular cell surface. Cytokines includes, but are not limited to, chemokines, interferons, interleukins, lymphokines, and tumor necrosis factors. Cytokines are produced by a wide range of cells, including immune cells, endothelial cells, fibroblasts, and stromal cells. A given cytokine may be produced by more than one cell type. Cytokine are pleiotropic; since the receptors are expressed on multiple immune cell subsets, one cytokine can activate the signaling pathway in multiple cells. However, depending on the cell type, the signaling events for a cytokine can result in different downstream cellular events such as activation, proliferation, survival, apoptosis, effector function and secretion of other immunomodulatory proteins.

“Amino acid” as used here refers to naturally occurring carboxy α-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

“Polypeptide” or “protein” as used here refers to a molecule where monomers (amino acids) are linearly linked to one another by peptide bonds (also known as amide bonds). The term “polypeptide” refers to any chain of two or more amino acids and does not refer to a specific length of the product. Thus, peptides, dipeptides, tripeptides, oligopeptides, “protein”, “amino acid chain”, or any other term used to refer to a chain of two or more amino acids, are included within the definition of “polypeptide”, and the term “polypeptide” may be used instead of, or interchangeably with any of these terms. The term “polypeptide” is also intended to refer to the products of A polypeptide may be derived from a natural biological source or produced by recombinant technology but is not necessarily translated from a designated nucleic acid sequence. It may be generated in any manner, including by chemical synthesis. Polypeptides normally have a defined three-dimensional structure, but they do not necessarily have such structure. A polypeptide of the present disclosure may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides with a defined three-dimensional structure are referred to as folded, and polypeptides which do not possess a defined three-dimensional structure, but rather can adopt many different conformations and are referred to as unfolded. Polypeptides may further form multimers such as dimers, trimers and higher oligomers, i.e. consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. The terms “polypeptide” and “protein” also refer to modified polypeptides/proteins wherein the post-expression modification is affected including without limitation glycosylation, acetylation, phosphorylation, amidation, derivatization by known protecting/blocking groups, proteolytic cleavage, or modification by non-naturally occurring amino acids.

“Identity” as used herein refers to the percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif, or can be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

“Residue” as used herein is meant a position in a protein and its associated amino acid identity. For example, Leu 234 (also referred to as Leu234 or L234) is a residue at position 234 in the human antibody IgG1.

“Wild-type” herein means an amino acid sequence or a nucleotide sequence that is found in nature, including allelic variations. A wild-type protein has an amino acid sequence or a nucleotide sequence that has not been intentionally modified.

“Substitution” refers to a change to a polypeptide or nucleic acid encoding said polypeptide, wherein an amino acid or nucleotide occurring naturally in the wild-type sequence of a polypeptide or nucleic acid is substituted to another amino acid or nucleotide not naturally occurring at the same position in the said polypeptide or nucleic acid sequence. In some embodiments, a substitution or substitutions are introduced to modify a polypeptide's affinity to its receptor thereby altering its activity such that it becomes different from the affinity and activity of the wild-type cognate polypeptide. In some embodiments, a substitution improves a polypeptide's biophysical properties. Amino acid substitutions can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful.

“Mutation” refers to a change to a polypeptide or nucleic acid encoding said polypeptide wherein: i) an amino acid and/or a nucleotide in the wildtype sequence have been substituted to another amino acid or nucleotide, ii) an amino acid and/or a nucleotide in the wildtype sequence have been deleted, and/or iii) an amino acid and/or a nucleotide has been inserted.

“CD8” as used here refers to any native human CD8. Unless otherwise indicated expressly or by context, references to “CD8” refer to CD8aa and/or CD8ab. The amino acid sequence of an exemplary human CD8b, the beta chain of human CD8, is described under UniProt P10966 (CD8B_HUMAN). “CD8a” refers to the alpha chain of human CD8 (e.g., as is described under UniProt P01732 (CD8A_HUMAN)). “CD8aa” refers to a homodimer of CD8a. “CD8ab” refers to a heterodimer of CD8a and CD8b. “CD8,” “CD8a,” “CD8b,” “CD8aa,” and “CD8ab” encompass unprocessed forms as well as mature forms that result from processing in the cell. “CD8,” “CD8a,” “CD8b,” “CD8aa,” and “CD8ab” also include but are not limited to naturally occurring variants, e.g. allelic or splice variants or variants.

“Interleukin-2” or “IL-2” as used here refers to any native human IL-2 or a variant thereof, unless otherwise indicated. In some cases, “IL-2” encompasses unprocessed IL-2 as well as “mature IL-2” which is a form of IL-2 that results from processing in the cell. One exemplary form of unprocessed native human IL-2 comprises of an additional N-terminal amino acid signal peptide attached to mature IL-2. “IL-2” also includes but is not limited to naturally occurring variants of IL-2, e.g. allelic or splice variants or variants. The amino acid sequence of an exemplary native human IL-2 is described under UniProt P60568 (IL2_HUMAN).

“Affinity” or “binding affinity” refers to the strength of the sum total of non-covalent interactions between a single binding site of a molecule (e.g. an antibody) and its binding partner (e.g. an antigen). Unless indicated otherwise, as used herein, “binding affinity” refers to intrinsic binding affinity which reflects a 1:1 interaction between members of a binding pair (e.g. antibody and antigen). The affinity can generally be represented by the dissociation constant (K_{D}), which is the ratio of dissociation and association rate constants (koff and kon, respectively). Thus, equivalent affinities may comprise different rate constants, as long as the ratio of the rate constants remains the same. Affinity can be measured by common methods known in the art, such as enzyme-linked immunosorbent assay (ELISA), surface plasmon resonance (SPR) technologies (e.g. BIAcore), BioLayer Interferometry (BLI) technologies (e.g. Octet) and other traditional binding assays (Heeley, Endocr Res 28, 217-229 (2002).

“Binding” or “specific binding” as used here, refers the ability of a polypeptide or an antigen binding molecule to selectively interact with the receptor for the polypeptide or target antigen, respectively, and this specific interaction can be distinguished from non-targeted or undesired or non-specific interactions. Examples of specific binding include but are not limited to IL-2 cytokine binding to its specific receptors (e.g. IL-2Rα, IL-2Rβ and IL-2Rγ) and an antigen binding molecule binding to a specific antigen (e.g. CD8 or PD-1).

“Mutant IL-2 polypeptide” refers to IL-2 polypeptide that has reduced affinity to its receptor wherein such decreased affinity will result in reduced biological activity of the mutant. Reduction in affinity and thereby activity can be obtained by introducing a small number of amino acid mutations or substitutions. The mutant IL-2 polypeptides can also have other modifications to the peptide backbone, including but not limited to amino acid deletion, permutation, cyclization, disulfide bonds, or the post-translational modifications (e.g. glycosylation or altered carbohydrate) of a polypeptide, chemical or enzymatic modifications to the polypeptide (e.g. attaching PEG to the polypeptide backbone), addition of peptide tags or labels, or fusion to proteins or protein domains to generate a final construct with desired characteristics, such as reduced affinity to IL-2Rβγ. Desired activity may also include improved biophysical properties compared to the wild-type IL-2 polypeptide. Multiple modifications may be combined to achieve desired activity modification, such as reduction in affinity or improved biophysical properties. As a non-limiting example, amino acid sequences for consensus N-link glycosylation may be incorporated into the polypeptide to allow for glycosylation. Another non-limiting example is that a lysine may be incorporated onto the polypeptide to enable pegylation. Preferably, a mutation or mutations are introduced to the polypeptide to modify its activity.

In some cases, “mutant IL-21 polypeptide” refers to IL-21 polypeptide that has reduced affinity to its receptor wherein such decreased affinity will result in reduced biological activity of the mutant. Reduction in affinity and thereby activity can be obtained by introducing a small number of amino acid mutations or substitutions. The mutant IL-21 polypeptides can also have other modifications to the peptide backbone, including but not limited to amino acid deletion, permutation, cyclization, disulfide bonds, or the post-translational modifications (e.g. glycosylation or altered carbohydrate) of a polypeptide, chemical or enzymatic modifications to the polypeptide (e.g. attaching PEG to the polypeptide backbone), addition of peptide tags or labels, or fusion to proteins or protein domains to generate a final construct with desired characteristics, such as reduced affinity to IL-21R. Desired activity may also include improved biophysical properties compared to the wild-type IL-21 polypeptide. Multiple modifications may be combined to achieve desired activity modification, such as reduction in affinity or improved biophysical properties. As a non-limiting example, amino acid sequences for consensus N-link glycosylation may be incorporated into the polypeptide to allow for glycosylation. Another non-limiting example is that a lysine may be incorporated onto the polypeptide to enable pegylation. Preferably, a mutation or mutations are introduced to the polypeptide to modify its activity.

The terms “antibody” and “immunoglobulin” are used interchangeably and herein are used in the broadest sense and encompasses various antibody structures, including but not limited to monoclonal antibodies (e.g., full length or intact monoclonal antibodies), polyclonal antibodies, multispecific antibodies (e.g. bispecific antibodies), antibody fragments and single domain antibody (as described in greater detail herein), so long as they exhibit the desired antigen binding activity.

Antibodies (immunoglobulins) refer to a protein having a structure substantially similar to a native antibody structure. “Native antibodies” refer to naturally occurring immunoglobulin molecules with varying structures. For example, native immunoglobulins of the IgG class are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The subunit structures and three-dimensional configurations of the different classes of immunoglobulins are well known and described generally, for example, in Abbas et al., 2000, Cellular and Mol, and Kindt et al., Kuby Immunology, 6th ed., W.H. Freeman and Co., page 91 (2007). Antibodies (immunoglobulins) are assigned to different classes, depending on the amino acid sequences of the heavy chain constant domains. There are five major classes of antibodies: α(IgA), δ (IgD), ε (IgE), γ (IgG), or μ (IgM), some of which may be further divided into subtypes, e.g. γ1 (IgG1), γ2 (IgG2), γ3 (IgG3), γ4 (IgG4), α1 (IgA1) and α2 (IgA2). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

“Fc” or “Fc region” or “Fc domain” as used herein refers to the C-terminal region of an antibody heavy chain that contains at least a portion of the constant region. The term includes native sequence Fc regions and variant Fc regions. An Fc can refer to the last two constant region immunoglobulin domains (e.g., CH2 and CH3) of IgA, IgD, and IgG, the last three constant region immunoglobulin domains of IgE and IgM, and optionally, all or a portion of the flexible hinge N-terminal to these domains. For IgA and IgM, Fc may include the J chain. An IgG Fc region comprises an IgG CH2 and an IgG CH3 domain and in some cases, inclusive of the hinge. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991. The “hinge” region usually extends from amino acid residue at about position 216 to amino acid residue at about position 230. The hinge region herein may be a native hinge domain or variant hinge domain. The “CH2 domain” of a human IgG Fc region usually extends from an amino acid residue at about position 231 to an amino acid residue at about position 340. The CH2 domain herein may be a native sequence CH2 domain or variant CH2 domain. The “CH3 domain” comprises the stretch of residues C-terminal to a CH2 domain in an Fc region, from an amino acid residue at about position 341 to an amino acid residue at about position 447 of an IgG. The CH3 region herein may be a native sequence CH3 domain or a variant CH3 domain (e.g. a CH3 domain with an introduced “protuberance” (“knob”) in one chain thereof and a corresponding introduced “cavity” (“hole”) in the other chain thereof; see U.S. Pat. No. 5,821,333, expressly incorporated herein by reference). Thus, the definition of “Fc domain” includes both amino acids 231-447 (CH2-CH3) or 216-447 (hinge-CH2-CH3), or fragments thereof. An “Fc fragment” in this context may contain fewer amino acids from either or both of the N- and C-termini but still retains the ability to form a dimer with another Fc domain or Fc fragment as can be detected using standard methods, generally based on size (e.g. non-denaturing chromatography, size exclusion chromatography, etc.). Human IgG Fc domains are of particular use in the present disclosure, and can be the Fc domain from human IgG1, IgG2 or IgG4.

A “variant Fc domain” or “Fc variant” or “variant Fc” contains amino acid modifications (e.g. substitution, addition, and deletion) as compared to a parental Fc domain. The term also includes naturally occurring allelic variants of the Fc region of an immunoglobulin. In general, variant Fc domains have at least about 80, 85, 90, 95, 97, 98 or 99 percent identity to the corresponding parental human IgG Fc domain (using the identity algorithms discussed below, with one embodiment utilizing the BLAST algorithm as is known in the art, using default parameters). Alternatively, the variant Fc domains can have from 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. For example, one or more amino acids can be deleted from the N-terminus or C-terminus of the Fc region of an immunoglobulin without substantial loss of biological function. Additionally, as discussed herein, the variant Fc domains herein still retain the ability to form a dimer with another Fc domain as measured using known techniques as described herein, such as non-denaturing gel electrophoresis.

“Fc gamma receptor”, “FcγR” or “Fc gamma R” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “effector function” as used herein is meant a biochemical event that results from the interaction of an antibody Fc region with an Fc receptor or ligand, which vary with the antibody isotype. Effector functions include but are not limited to antibody-dependent cell-mediated cytotoxicity (ADCC), antibody-dependent cell-mediated phagocytosis (ADCP), complement-dependent cytotoxicity (CDC), cytokine secretion, immune complex-mediated antigen uptake by antigen presenting cells, down regulation of cell surface receptors (e.g. B cell receptor), and B cell activation. “Antibody-dependent cell-mediated cytotoxicity” or “ADCC” refer to a cell-mediated reaction in which nonspecific cytotoxic cells that express FcRs (such as Natural Killer (NK) cells, neutrophils, and macrophages) recognize bound antibody on a target cell and subsequently cause lysis of the target cell. ADCC is correlated with binding to FcγRIIIa; increased binding to FcγRIIIa leads to an increase in ADCC activity. To assess ADCC activity of a molecule of interest, an in vitro ADCC assay such as that described in U.S. Pat. No. 5,500,362 or 5,821,337 may be performed. “ADCP” or antibody dependent cell-mediated phagocytosis as used herein is meant the cell-mediated reaction wherein nonspecific cytotoxic cells that express FcγRs recognize bound antibody on a target cell and subsequently cause phagocytosis of the target cell.

“Fc null” and “Fc null variant” are used interchangeably and used herein to describe a modified Fc which have reduced or abolished effector functions. Such Fc null or Fc null variant have reduced or abolished to FcγRs and/or complement receptors. Preferably, such Fc null or Fc null variant has abolished effector functions. Exemplary methods for the modification include but not limited to chemical alteration, amino acid residue substitution, insertion and deletions. Exemplary amino acid positions on Fc molecules where one or more modifications were introduced to decrease effector function of the resulting variant (numbering based on the EU numbering scheme) at position i) IgG1: C220, C226, C229, E233, L234, L235, G237, P238, S239 D265, S267, N297, L328, P331, K322, A327 and P329, ii) IgG2: V234, G237, D265, H268, N297, V309, A330, A331, K322 and iii) IgG4: L235, G237, D265 and E318. Exemplary Fc molecules having decreased effector function include those having one or more of the following substitutions: i) IgG1: N297A, N297Q, N297G, D265A/N297A, D265A/N297Q, C220S/C226S/C229S/P238S, S267E/L328F, C226S/C229S/E233P/L234V/L235A, L234F/L235E/P331S, L234A/L235A, L234A/L235A/G237A, L234A/L235A/G237A/K322A, L234A/L235A/G237A/A330S/A331S, L234A/L235A/P329G,E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del, L234A/L235A/G237deleted; ii) IgG2: A330S/A331S, V234A/G237A, V234A/G237A/D265A, D265A/A330S/A331S, V234A/G237A/D265A/A330S/A331S, and H268Q/V309L/A330S/A331S; iii) IgG4: L235A/G237A/E318A, D265A, L235A/G237A/D265A and L235A/G237A/D265A/E318A.

“Epitope” as used herein refers to a determinant capable of specific binding to the variable region of an antibody molecule known as a paratope. Epitopes are groupings of molecules such as amino acids or sugar side chains and usually have specific structural characteristics, as well as specific charge characteristics. A single antigen may have more than one epitope. The epitope may comprise amino acid residues directly involved in the binding and other amino acid residues, which are not directly involved in the binding, such as amino acid residues which are effectively blocked by the antigen binding peptide (in other words, the amino acid residue is within the footprint of the antigen binding peptide). Epitopes may be either conformational or linear. An epitope typically includes at least 3, and more usually, at least 5 or 8-10 amino acids. Antibodies that recognize the same epitope can be verified in a simple immunoassay showing the ability of one antibody to block the binding of another antibody to a target antigen, for example “binning”.

“Linker” as used herein refers to a molecule that connect two polypeptide chains. Linker can be a polypeptide linker or a synthetic chemical linker (for example, see disclosed in Protein Engineering, 9(3), 299-305, 1996). The length and sequence of the polypeptide linkers is not particularly limited and can be selected according to the purpose by those skilled in the art. Polypeptide linker comprises one or more amino acids. In some embodiments, the polypeptide linker is a peptide with a length of at least 5 amino acids, preferably with a length of 5 to 100, more preferably of 10 to 50 amino acids. In one embodiment, said peptide linker is G, S, GS, SG, SGG, GGS, and GSG (with G=glycine and S=serine). In another embodiment, said peptide linker is (GGGS)xGn (SEQ ID NO: 74) or (GGGGS)xGn (SEQ ID NO: 75) or (GGGGGS)xGn (SEQ ID NO: 76) or S(GGGS)xGn (SEQ ID NO: 386) or S(GGGGS)xGn (SEQ ID NO: 387) or S(GGGGGS)xGn (SEQ ID NO: 388), with x=1, 2, 3, 4, 5, 6, 7, 8, 9, 10, 11, or 12 and n=0, 1, 2 or 3. Preferably, the said linker is (GGGGS)xGn with x=2, 3, or 4 and n=0 (SEQ ID NO: 77); more preferably the said linker is (GGGGS)xGn with x=3 and n=0 (SEQ ID NO: 78). In some embodiments, the linker comprises the sequence GGGGSGGGGSGGGGS (SEQ ID NO: 79) or or SGGGGSGGGGSGGGGS (SEQ ID NO: 389). Synthetic chemical linkers include crosslinking agents that are routinely used to crosslink peptides, for example, N-hydroxy succinimide (NHS), disuccinimidyl suberate (DSS), bis(succinimidyl) suberate (BS3), dithiobis(succinimidyl propionate) (DSP), dithiobis(succinimidyl propionate) (DTSSP), ethylene glycol bis(succinimidyl succinate) (EGS), ethylene glycol bis(sulfosuccinimidyl succinate) (sulfo-EGS), disuccinimidyl tartrate (DST), disulfosuccinimidyl tartrate (sulfo-DST), bis[2-(succinimidoxycarbonyloxy)ethyl]sulfone (BSOCOES), and bis[2-(succinimidoxycarbonyloxy)ethyl]sulfone (sulfo-BSOCOES).

The term “polynucleotide” refers to an isolated nucleic acid molecule or construct, e.g. messenger RNA (mRNA), virally-derived RNA, or plasmid DNA (pDNA) encoding the polypeptides of the present disclosure. A polynucleotide may comprise a conventional phosphodiester bond or a non-conventional bond (e.g. an amide bond, such as found in peptide nucleic acids (PNA). The term “nucleic acid molecule” refers to any one or more nucleic acid segments, e.g. DNA or RNA fragments, present in a polynucleotide. In some aspects, one or more vectors (particularly expression vectors) comprising such nucleic acids are provided. In one aspect, a method for making a polypeptide of the present disclosure is provided, wherein the methods comprises culturing a host cell comprising a nucleic acid encoding the polypeptide under conditions suitable for expression of the polypeptide and recovering the polypeptide from the host cell. “Recombinant” means the proteins are generated using recombinant nucleic acid techniques in exogeneous host cells. Recombinantly produced proteins expressed in host cells are considered isolated for the purpose of the present disclosure, as are native or recombinant proteins which have been separated, fractionated, or partially or substantially purified by any suitable technique.

“Isolated,” when used to describe the various polypeptides disclosed herein, means a polypeptide that has been identified and separated and/or recovered from a cell or cell culture from which it was expressed. Typically, an isolated polypeptide will be purified by at least one purification step. There is no required level of purity; “purification” or “purified” refers to increase of the target protein concentration relative to the concentration of contaminants in a composition as compared to the starting material. An “isolated protein,” as used herein refers to a target protein which is substantially free of other proteins having different binding specificities.

The term “cancer” refers to the physiological condition in mammals that is typically characterized by unregulated and abnormal cell growth with the potential to invade or spread to other parts of the body. Examples of cancer include but are not limited to, carcinoma, lymphoma, blastoma, sarcoma, and leukemia. More particular examples of such cancers include lung cancer, small-cell lung cancer, non-small cell lung (NSCL) cancer, bronchioloalviolar cell lung cancer, squamous cell cancer, adenocarcinoma of the lung, squamous carcinoma of the lung, cancer of the peritoneum, head and neck cancer, bone cancer, pancreatic cancer, skin cancer, cancer of the head or neck, cutaneous or intraocular melanoma, thyroid cancer, uterine cancer, gastrointestinal cancer, ovarian cancer, rectal cancer, cancer of the anal region, stomach cancer, gastric cancer, colon cancer, breast cancer, endometrial carcinoma, uterine cancer, carcinoma of the fallopian tubes, carcinoma of the cervix, carcinoma of the vagina, vulval cancer, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, bladder cancer, liver cancer, hepatoma, hepatocellular cancer, cervical cancer, salivary gland carcinoma, biliay cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma and Ewings sarcoma, including refractory versions of any of the above cancers, or a combination of one or more of the above cancers.

In some cases, the terms “combination treatment” or “combination therapy” refer to administration of two or more active agents or therapies in a regimen that will provide beneficial effects of the combination, including coadministration of active agents or therapies in a substantially simultaneous manner, such as in a single formulation having a fixed ratio of active agents or in co-administration in a sequential manner, such as in multiple, separate formulations of each active agent or therapy. Combination therapy also includes combinations where individual elements (e.g., active ingredients or therapies) may be administered at different times and/or by different routes but which act in combination to provide a beneficial effect by co-action or pharmacokinetic and pharmacodynamics effect of each agent or treatment approaches of the combination therapy.

### Fusion Proteins

Provided herein are IL-2 fusion proteins and IL-21 fusion proteins comprising any one of the anti-CD8 antibodies, or antigen binding domains, or antibody fragments disclosed herein. In some embodiments, a fusion protein of the present disclosure comprises a first moiety comprising a human or humanized antibody or antigen-binding fragment thereof that specifically binds CD8 (e.g., any one of the anti-CD8 antibodies described infra) and a second moiety comprising IL-2 or IL-21. In some embodiments, the first moiety is fused to the second moiety directly. In some embodiments, the first moiety is fused to the second moiety via a linker.

In some embodiments, a fusion protein described herein induces activation of cells expressing a human CD8ab heterodimer with at least 2-fold, at least 5-fold, or at least 10-fold higher potency than activation of cells expressing a human CD8aa homodimer. In some embodiments, the fusion protein induces activation of CD8+ T cells with at least 2-fold, at least 5-fold, or at least 10-fold higher potency than activation of NK cells. In some embodiments, potency of activation is measured by EC50, as assessed by cell proliferation. Exemplary assays are further described herein.

### Fusion Protein Formats

In some embodiments, the first moiety comprises an antibody (e.g., an anti-CD8 antibody of the present disclosure). In some embodiments, the first moiety comprises an antibody fragment (e.g., an anti-CD8 antibody fragment of the present disclosure). In some embodiments, the first moiety comprises a single chain antibody or single chain variable fragment (scFv). In some embodiments, the first moiety comprises a VHH antibody. In some embodiments, the first moiety comprises one or two antibody heavy chain polypeptides and one or two antibody light chain polypeptides (e.g., of an anti-CD8 antibody of the present disclosure). In some embodiments, the first moiety comprises the 3 heavy chain CDRs and/or 3 light chain CDRs of a single anti-CD8 antibody of the present disclosure, e.g., as shown in Tables 1-3. In some embodiments, the first moiety comprises the VH and/or VL domain(s) of a single anti-CD8 antibody of the present disclosure, e.g., as shown in Table 3. In some embodiments, the first moiety further comprises one or two human IgG Fc domains. In some embodiments, the one or two human IgG Fc domains are IgG1, IgG2, IgG3 or IgG4 Fc domains. In some embodiments, the one or two human IgG Fc domains do not have the C-terminus lysine residue. In some embodiments, the one or two human IgG Fc domains comprise amino acid modifications (such as substitutions, deletions, additions, etc.). In some embodiments, the Fc domain modifications promote heterodimeric formation (e.g., as shown in Table 4). In some embodiments, the one or two Fe domains comprise Fe gamma-null mutations.

In some embodiments, the first moiety comprises two antibody heavy chain polypeptides comprising a structure according to formula [I], from N-terminus to C-terminus:

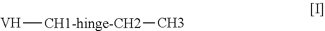

and two antibody light chain polypeptides comprising a structure according to formula [II], from N-terminus to C-terminus:

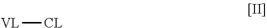

wherein VH is the VH domain, wherein CH1 is an antibody CH1 domain, wherein hinge is an antibody hinge domain, wherein CH2-CH3 is an antibody Fc domain, wherein VL is the VL domain, and wherein CL is an antibody constant light chain domain. In some embodiments, the N-terminus of the second moiety is fused to the C-terminus of one of the two CH3 domains.

In some embodiments, the first moiety comprises a first antibody heavy chain polypeptide comprising a structure according to formula [I], from N-terminus to C-terminus:

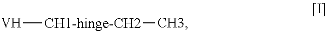

an antibody light chain polypeptide comprising a structure according to formula [II], from N-terminus to C-terminus:

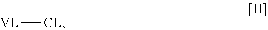

and a second antibody heavy chain polypeptide comprising a structure according to formula [III], from N-terminus to C-terminus:

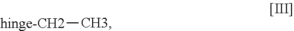

wherein VH is the VH domain, wherein CH1 is an antibody CH1 domain, wherein hinge is an antibody hinge domain, wherein CH2-CH3 is an antibody Fc domain, wherein VL is the VL domain, and wherein CL is an antibody constant light chain domain. In some embodiments, the N-terminus of the second moiety is fused to the C-terminus of the CH3 domain of the second antibody heavy chain polypeptide. In some embodiments, the N-terminus of the second moiety is fused to the C-terminus of the CH3 domain of the first antibody heavy chain polypeptide.

In some embodiments, the first moiety comprises a first antibody heavy chain polypeptide comprising a structure according to formula [I], from N-terminus to C-terminus:

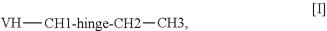

an antibody light chain polypeptide comprising a structure according to formula [II], from N-terminus to C-terminus:

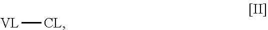

and a second antibody heavy chain polypeptide comprising a structure according to formula [III], from N-terminus to C-terminus:

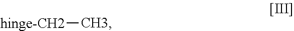

wherein VH is the VH domain, wherein CH1 is an antibody CH1 domain, wherein hinge is an antibody hinge domain, wherein CH2-CH3 is an antibody Fc domain, wherein VL is the VL domain, and wherein CL is an antibody constant light chain domain. In some embodiments, the C-terminus of the second moiety is fused to the N-terminus of the hinge domain of the second antibody heavy chain polypeptide.

In some embodiments, an anti-CD8 antibody of the present disclosure is a multispecific (e.g., bispecific) antibody or antibody fragment. For example, in some embodiments, the multispecific antibody (e.g., bispecific antibody) comprises a first antigen binding domain that binds to CD8 (e.g., as described supra) and a second antigen binding domain that binds a target of interest. In some embodiments, a bispecific antibody can be generated via fusion of an additional binding site to either the heavy or light chain of an immunoglobulin. Examples of the additional binding site include but not limited to variable regions, scFv, Fab, VHH, and peptide.

In some embodiments, the recombinant bispecific antibodies disclosed herein can be very roughly classified in two categories, namely i) formats resulting from the combination of variable regions only and ii) formats combining variable regions with Fc domains. Representatives of the first category are tandem scFv (taFv), diabodies (Db), DART, single-chain diabodies (scDbs), Fab-Fc, tandem Fab, Dual variable region Fab and tandem dAb/VHH. The two variable regions can be linked together via covalent bonds or non-covalent interaction.

Noncovalent interaction may involve the use of heterodimerization modules such as leucine zipper, dock-and-lock methods of using regulatory subunit of cAMP-dependent protein kinase (PKA) and the anchoring domains of A kinase anchor proteins (AKAPs) or knob-into-holes CH3 domain (U.S. Pat. Nos. 5,731,168; 7,695,936; Ridgway et al., Prot Eng 9, 617-621 (1996) and Carter, J Immunol Meth 248, 7-15 (2001)) to pair up the variable regions.

In some embodiments, bispecific antibodies are generated on the natural immunoglobulin architecture containing two pairs of heavy chain and light chain combination with each pair having distinct binding specificity. Homodimerization of the two heavy chains in an IgG is mediated by the CH3 interaction. To promote heterodimeric formation, genetic modifications are introduced to the two respective CH3 regions. There heterodimerization mutations often involve steric repulsion, charge steering interaction, or interchain disulfide bond formation. Exemplary and non-limiting Fc modifications to promote heterodimerization include the following:

TABLE 4
Exemplary Fc modifications to promote heterodimerization.

| | CH3 domain | CH3 domain | |
|---|---|---|---|
| Strategy | 1 | 2 | References |
| knobs-into-holes 1 | T366Y | Y407T | Brinkmann & |
| knobs-into-holes 2 | T366W | T366S- | Kontermann, MAbs. |
| | | L368A- | 2017 Feb-Mar; 9(2): |
| | | Y407V | 182-212; |
| knobs-into-holes 3 | S354C, | Y349C, | Atwell et al, J Mol |
| | T366W | T366S, | Biol 1997; 270: 26-35; |
| | | L368A, | Merchant et |
| | | Y407V | al, Nat Biotechnol |
| knobs-into-holes 4 | Y349C, | S354C, | 1998; 16: 677- |
| | T366W | T366S, | 681 |
| | | L368A, | |
| | | Y407V | |
| HA-TF | S364H, | Y349T, | Moore et al, MAbs |
| | F405A | T394F | 2011; 3: 546- |
| | | | 557 |
| ZW1 | T350V, | T350V, | Von Kreudenstein et |
| | L351Y, | T366L, | al, MAbs 2013; 5: 646- |
| | F405A, | K392L, | 54 |
| | Y407V | T394W | |
| CH3 charge pairs (DD-KK) | K392D, | E356K, | Gunasekaran et al, J |
| | K409D | D399K | Biol |
| | | | Chem 2010; |
| | | | 285: 19637-46 |
| IgG1 hinge/CH3 charge pairs | IgG1: | IgG1: | Strop P et al, J Mol |
| (EEE-RRR) | D221E, | D221R, | Biol 2012; 420: 204-19 |
| | P228E, | P228R, | |
| | L368E | K409R | |
| IgG2 hinge/CH3 charge pairs | IgG2: | IgG2: | |
| (EEE-RRRR) | C223E, | C223R, | |
| | P228E, | E225R, | |
| | L368E | P228R, | |
| | | K409R | |
| EW-RVT | K360E, | Q347R, | Choi et al, Mol Cancer |
| | K409W, | D399V, | Ther 2013; 12: 2748-59 |
| | | F405T | |
| EW-RVT_{S-S} | K360E, | Q347R, | Choi et al, Mol |
| | K409W, | D399V, | Immunol 2015; |
| | Y349C | F405T, | 65: 377-83 |
| | | S354C | |
| Biclonic | 366K | 351D or E or | Geuijen et al, ournal of |
| | (+351K) | D at 349, | Clinical Oncology |
| | | 368, 349, or | 2014; 32: suppl: 560 |
| | | 349 + 355 | |
| DuoBody (L-R) 1 | F405L | K409R | Labrijn et al, Nat |
| DuoBody (L-R) 2 | F405L- | WT (R409) | Protoc 2014; 9: 2450- |
| | R409K | | 63; Labrijn et al, |
| | | | PNAS 2013; |
| | | | 110(13): 5145-50 |
| SEEDbody | IgG/A | IgG/A | Davis et al, Protein |
| | chimera | chimera | Eng Des |
| | | | Sel 2010; 23: 195-202 |
| BEAT | residues | residues | Moretti et al, BMC |
| | from TCRα | from TCRβ | Proceedings 2013; |
| | interface | interface | 7(Suppl 6): O9 |
| Mixed interface (MI) | IgG-CH3 | igA/D/M | Skegro et al, J Biol |
| heterodimers | variants | CH3 variants | Chem. 2017 292(23): |
| | | or IgM CH4 | 9745-9759. |
| | | variants | |
| XmAb | E357Q- | L368D- | Moore et al, Methods. |
| | S364K | K370S | 2019 |
| | | | 154: 38-50 |
| DEKK Fc | L351D- | L351K- | De Nardis et al., J Biol |
| | L368E | T366K | Chem. 2017; 292(35): |
| | | | 14706-14717. |
| Charge pair | E356K or | K370E, | Igawa T, Tsunoda H. |
| | E357K or | K409D, | WO2006106905. 2006. |
| | D399K | K439E | |
| KKA-DDW | D356K- | K392D- | Zhou et al, |
| | D399K- | K409D- | WO2014079000A1 |
| | Y407A | T366W | |
| Charge pair | L368E- | E357K- | Labrijn et al, Nat |
| | Y407E | D399K | Review Drug |
| | | | Discovery 2019; 18, |
| | | | 585-608 |
| Knob-hole-electrostatic | S354C- | Y349C- | Wei et al., Oncotarget. |
| | T366W- | T366S- | 2017; 8(31): 51037- |
| | K409A | L368A- | 51049 |
| | | Y407- | |
| | | F405K | |
| KA | F405K | K409A | Wei et al., Oncotarget. |
| | | | 2017; 8(31): 51037- |
| | | | 51049 |
| PPV-TPP | P395K- | T394D- | Wenjun Zhang |
| | P396K- | P395D- | US10538595B2 |
| | V397K, | P396D, | |
| | P395K- | T394C- | |
| | P396K- | P395D- | |
| | V397C or | P396D or | |
| | P395R- | T394E- | |
| | P396R- | P395E- | |
| | V397R | P396E | |

In some embodiments, bispecific antibody can be generated by post-production assembly from half-antibodies, thereby solving the issues of heavy and light chain mispairing. These antibodies often contain modification to favor heterodimerization of half-antibodies. Exemplary systems include but not limited to the knob-into-hole, IgG1 (EEE-RRR), IgG2 (EEE-RRRR) (Strop et al. J Mol Biol (2012)) and DuoBody (F405L-K409R), listed in Table4. In such case, half-antibody is individually produced in separate cell line and purified. The purified antibodies were then subjected to mild reduction to obtain half-antibodies, which were then assembled into bispecific antibodies. Heterodimeric bispecific antibody was then purified from the mixture using conventional purifications methods.

In some embodiments, strategies on bispecific antibody generation that do not rely on the preferential chain pairing can also be employed. These strategies typically involve introducing genetic modification on the antibody in such a manner that the heterodimer will have distinct biochemical or biophysical properties from the homodimers; thus the post-assembled or expressed heterodimer can be selectively purified from the homodimers. One example was to introduce H435R/Y436F in IgG1 CH3 domain to abolish the Fc binding to protein A resin and then co-express the H435R/Y436F variant with a wildtype Fc. The resulting homodimeric antibodies containing two copies of H435R/Y436F cannot bind to the Protein A column, while heterodimeric antibody comprising one copy of H435R/Y436F mutation will have a decreased affinity for protein A as compared to the strong interaction from homodimeric wildtype antibody (Tustian et al Mabs 2016). Other examples include kappa/lambda antibody (Fischer et al., Nature Communication 2015) and introduction of differential charges (E357Q, S267K or N208D/Q295E/N384D/Q418E/N421D) on the respective chains (US 2018/0142040 A1; (Strop et al. J Mol Biol (2012)).

In some embodiments, bispecific antibody can be generated via fusion of an additional binding site to either the heavy or light chain of an immunoglobulin. Examples of the additional binding site include but not limited to variable regions, scFv, Fab, VHH, and peptide.

### Fusion Protein Fc Regions

In some embodiments, an antibody or fusion protein of the present disclosure comprises an Fc region. In some embodiments, the Fc region comprises one or more mutations that reduce or eliminate FcγR binding and/or effector function. In some embodiments, the Fc region (e.g., an IgG1 Fc region) comprises a substitution at one or more of the following positions: C220, C226, C229, E233, L234, L235, G237, P238, S239 D265, S267, N297, L328, P331, K322, A327 and P329. In some embodiments, the Fc region (e.g., an IgG2 Fc region) comprises a substitution at one or more of the following positions: V234, G237, D265, H268, N297, V309, A330, A331, K322. In some embodiments, the Fc region (e.g., an IgG4 Fc region) comprises a substitution at one or more of the following positions: L235, G237, D265 and E318. In some embodiments, the Fc region (e.g., an IgG1 Fc region) comprises one or more of the following mutations or groups of mutations: N297A, N297Q, N297G, D265A/N297A, D265A/N297Q, C220S/C226S/C229S/P238S, S267E/L328F, C226S/C229S/E233P/L234V/L235A, L234F/L235E/P331S, L234A/L235A, L234A/L235A/G237A, L234A/L235A/G237A/K322A, L234A/L235A/G237A/A330S/A331S, L234A/L235A/P329G,E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del, L234A/L235A/G237deleted. In some embodiments, the Fc region (e.g., an IgG2 Fc region) comprises one or more of the following mutations or groups of mutations: A330S/A33IS, V234A/G237A, V234A/G237A/D265A, D265A/A330S/A331S, V234A/G237A/D265A/A330S/A331S, and H268Q/V309L/A330S/A331S. In some embodiments, the Fc region (e.g., an IgG4 Fc region) comprises one or more of the following mutations or groups of mutations: L235A/G237A/E318A, D265A, L235A/G237A/D265A and L235A/G237A/D265A/E318A. In some embodiments, the Fc region comprises one, two, three, or all of the following mutations: L234A, L235A, G237A, and K322A, numbering according to EU index. In some embodiments, the Fc region comprises one, two, or all of the following mutations: L234A, L235A, and G237A, numbering according to EU index.

In some embodiments, said first and second Fe domains of the fusion protein contain one or more of the following Fc mutations to decrease effector function according to EU numbering: L234A, L235A, G237A, and K322A. In some embodiments, said first and second Fe domains of the fusion protein contain the following Fc mutations to decrease effector function according to EU numbering: L234A, L235A, and G237A. In some embodiments, said first and second Fc domains of the fusion protein contain the following Fc mutations to decrease effector function according to EU numbering: L234A, L235A, G237A, and K322A. In some embodiments, said first and second Fc domains of the fusion protein contain the following amino acid substitutions to facilitate heterodimeric formation: Y349C/T366W (knob) and S354C, T366S, L368A and Y407V (hole).

In some embodiments, the heterodimeric mutations and/or mutations to modify Fc gamma receptor binding resulted in reduction of Fc stability. Therefore, additional mutation(s) was added to the Fc region to increase its stability. For example, one or more pairs of disulfide bonds such as A287C and L306C, V259C and L306C, R292C and V302C, and V323C and 1332C are introduced into the Fc region. Another example is to introduce S228P to IgG4 based bispecific antibodies to stabilize the hinge disulfide. Additional example includes introducing K3381, A339K, and K340S mutations to enhance Fc stability and aggregation resistance (Gao et al, 2019 Mol Pharm. 2019; 16:3647).

### Antibodies and Antigen-Binding Domains

In some embodiments, the fusion proteins described herein comprise an antibody, antibody fragment, or antigen binding domain that specifically binds human CD8b and/or human CD8ab. Any of the anti-CD8 antibodies of the present disclosure (e.g., that specifically bind human CD8b and/or human CD8ab) may find use in the fusion proteins, methods, and uses disclosed herein.

In some embodiments, the anti-CD8 antibody of the present disclosure specifically binds human CD8b and/or human CD8ab with at least 10-fold, at least 20-fold, at least 30-fold, at least 40-fold, at least 50-fold, at least 60-fold, at least 70-fold, at least 80-fold, at least 90-fold, at least 100-fold, or at least 200-fold higher affinity than its binding to human CD8a and/or human CD8aa, e.g., as expressed on natural killer (NK) cells (e.g., human NK cells). In some embodiments, the anti-CD8 antibody of the present disclosure specifically binds human CD8b and/or human CD8ab with at least 10-fold higher affinity than its binding to human CD8a and/or human CD8aa, e.g., as expressed on natural killer (NK) cells. In some embodiments, the human CD8b and/or human CD8ab are expressed on the surface of a human cell, e.g., a human T cell.

In some embodiments, the anti-CD8 antibody of the present disclosure specifically binds to a cell expressing a human CD8ab heterodimer on its surface (e.g., a human T cell) with an EC50 that is less than 1000 nM. In some embodiments, the anti-CD8 antibody of the present disclosure specifically binds to human CD8+ T cells.

In some embodiments, the anti-CD8 antibody of the present disclosure is a human antibody or antibody fragment. In some embodiments, a human antibody or antibody fragment comprises human-derived CDRs and framework sequences in the variable domain, e.g., as isolated from a human or generated using a library with human antibody sequences (e.g., CDR sequences). In some embodiments, the anti-CD8 antibody of the present disclosure is a humanized antibody or antibody fragment. In some embodiments, a humanized antibody or antibody fragment comprises non-human-derived CDRs (e.g., from a mouse, rabbit, goat, etc.) and human-derived framework sequences in the variable domain. In some embodiments, a human or humanized antibody further comprises a human Fc region. In some embodiments, the human Fc region further comprises one or more Fc mutations, e.g., as disclosed herein. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy chain constant domains that correspond to the different classes of antibodies are called α, δ, ε, γ, and μ, respectively. The subunit structures and three-dimensional configurations of different classes of immunoglobulins are well known.

Multiple definitions for the CDR sequences of antibody variable domains are known in the art. Unless otherwise specified, CDR sequences are described herein according to the definition of Kabat (see, e.g., Kabat et al., Sequences of Proteins of Immunological Interest, Fifth Edition, NIH Publication 91-3242, Bethesda MD (1991), vols. 1-3). However, other definitions are known and contemplated for use. For example, in some embodiments, CDR sequences can be described by the definition of Chothia (see, e.g., Chothia and Lesk, J. Mol. Biol. 196:901-917 (1987). Depending upon the specific CDR definition used, the precise framework sequences can also vary, but, as is known in the art, the first framework sequence (FW-1) refers to the sequence from the N-terminus of the VH or VL domain to the beginning of CDR-H1/-L1, the second framework sequence (FW-2) refers to the sequence from end of CDR-H1/-L1 to the beginning of CDR-H2/-L2, the third framework sequence (FW-3) refers to the sequence from end of CDR-H2/-L2 to the beginning of CDR-H3/-L3, and the fourth framework sequence (FW-4) refers to the sequence from end of CDR-H3/-L3 to the C-terminal boundary of the VH or VL domain.

Other numbering systems known in the art and are suitable for use in numbering the CDRs of the VH and/or VL amino acid sequences described herein. For example, the IMGT (INTERNATIONAL IMMUNOGENETICS INFORMATION SYSTEM) numbering of variable regions is the numbering of the residues in an immunoglobulin variable heavy or light chain according to the methods of the IMGT, as described in Lefranc, M.-P., “The IMGT unique numbering for immunoglobulins, T cell Receptors and Ig-like domains”, The Immunologist, 7, 132-136 (1999), and is expressly incorporated herein in its entirety by reference. As used herein, “IMGT sequence numbering” or “numbered according to IMGT,” refers to numbering of the sequence encoding a variable region according to the IMGT. For the heavy chain variable domain, when numbered according to IMGT, the hypervariable region ranges from amino acid positions 27 to 38 for CDR1, amino acid positions 56 to 65 for CDR2, and amino acid positions 105 to 117 for CDR3. For the light chain variable domain, when numbered according to IMGT, the hypervariable region ranges from amino acid positions 27 to 38 for CDR1, amino acid positions 56 to 65 for CDR2, and amino acid positions 105 to 117 for CDR3. When the CDRs are numbered according to IMGT, the heavy chain framework (FR) residues are positioned about at residues 1-26 (HC-FR1), 39-55 (HC-FR2), 66-104 (HC-FR3), and 118-128 (HCFR4) in the heavy chain residues and the light chain FR residues are positioned at about residues 1-26 (LC-FR1), 39-55 (LC-FR2), 66-104 (LC-FR3), and 118-128 (LC-FR4).

In some embodiments, an anti-CD8 antibody of the present disclosure comprises a VH domain comprising a CDR-H1 comprising the amino acid sequence of SEQ ID NO: 1, a CDR-H2 comprising the amino acid sequence of SEQ ID NO: 2, and a CDR-H3 comprising the amino acid sequence of SEQ ID NO: 3 and a VL domain comprising a CDR-L1 comprising the amino acid sequence of SEQ ID NO: 4, a CDR-L2 comprising the amino acid sequence of SEQ ID NO: 5, and a CDR-L3 comprising the amino acid sequence of SEQ ID NO: 6. In some embodiments, an anti-CD8 antibody of the present disclosure comprises 1, 2, or 3 heavy chain CDRs of antibody xhCD8v1 (e.g., as shown in Tables 1-3) and/or 1, 2, or 3 light chain CDRs of antibody xhCD8v1 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO: 58 and a VL domain comprising a CDR-L1, CDR-L2, and CDR-L3 from the sequence of SEQ ID NO: 59.

In some embodiments, an anti-CD8 antibody of the present disclosure comprises a VH domain comprising a CDR-H1 comprising the amino acid sequence of SEQ ID NO: 177, a CDR-H2 comprising the amino acid sequence of SEQ ID NO: 178, and a CDR-H3 comprising the amino acid sequence of SEQ ID NO: 179 and a VL domain comprising a CDR-L1 comprising the amino acid sequence of SEQ ID NO: 180, a CDR-L2 comprising the amino acid sequence of SEQ ID NO: 181, and a CDR-L3 comprising the amino acid sequence of SEQ ID NO: 182. In some embodiments, an anti-CD8 antibody of the present disclosure comprises 1, 2, or 3 heavy chain CDRs of antibody xhCD8v8 (e.g., as shown in Tables 1-3) and/or 1, 2, or 3 light chain CDRs of antibody xhCD8v8 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO: 185 and a VL domain comprising a CDR-L1, CDR-L2, and CDR-L3 from the sequence of SEQ ID NO: 186.

In some embodiments, an anti-CD8 antibody of the present disclosure comprises a VH domain comprising a CDR-H1 comprising the amino acid sequence of SEQ ID NO: 13, a CDR-H2 comprising the amino acid sequence of SEQ ID NO: 14, and a CDR-H3 comprising the amino acid sequence of SEQ ID NO: 15 and a VL domain comprising a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 18. In some embodiments, the VH domain comprises an amino acid sequence that is at least 90%, at least 95%, at least 99%, or 100% identical to the sequence of SEQ ID NO: 62 and/or the VL domain comprises an amino acid sequence that is at least 90%, at least 95%, at least 99%, or 100% identical to the sequence of SEQ ID NO: 63. In some embodiments, an anti-CD8 antibody of the present disclosure comprises 1, 2, or 3 heavy chain CDRs of antibody xhCD8v2 (e.g., as shown in Tables 1-3) and/or 1, 2, or 3 light chain CDRs of antibody xhCD8v2 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO: 62 and a VL domain comprising a CDR-L1, CDR-L2, and CDR-L3 from the sequence of SEQ ID NO: 63.

In some embodiments, an anti-CD8 antibody of the present disclosure comprises a VH domain comprising a CDR-H1 comprising the amino acid sequence of SEQ ID NO: 19, a CDR-H2 comprising the amino acid sequence of SEQ ID NO: 20, and a CDR-H3 comprising the amino acid sequence of SEQ ID NO: 21 and a VL domain comprising a CDR-L1 comprising the amino acid sequence of SEQ ID NO: 22, a CDR-L2 comprising the amino acid sequence of SEQ ID NO: 23, and a CDR-L3 comprising the amino acid sequence of SEQ ID NO: 24. In some embodiments, the VH domain comprises an amino acid sequence that is at least 90%, at least 95%, at least 99%, or 100% identical to the sequence of SEQ ID NO: 64 and/or the VL domain comprises an amino acid sequence that is at least 90%, at least 95%, at least 99%, or 100% identical to the sequence of SEQ ID NO: 65. In some embodiments, an anti-CD8 antibody of the present disclosure comprises 1, 2, or 3 heavy chain CDRs of antibody xhCD8v3 (e.g., as shown in Tables 1-3) and/or 1, 2, or 3 light chain CDRs of antibody xhCD8v3 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO: 64 and a VL domain comprising a CDR-L1, CDR-L2, and CDR-L3 from the sequence of SEQ ID NO: 65.

In some embodiments, an anti-CD8 antibody of the present disclosure comprises a VH domain comprising a CDR-H1 comprising the amino acid sequence of SEQ ID NO: 25, a CDR-H2 comprising the amino acid sequence of SEQ ID NO: 26, and a CDR-H3 comprising the amino acid sequence of SEQ ID NO: 27 and a VL domain comprising a CDR-L1 comprising the amino acid sequence of SEQ ID NO: 28, a CDR-L2 comprising the amino acid sequence of SEQ ID NO: 29, and a CDR-L3 comprising the amino acid sequence of SEQ ID NO: 30. In some embodiments, the VH domain comprises an amino acid sequence that is at least 90%, at least 95%, at least 99%, or 100% identical to the sequence of SEQ ID NO: 66 and/or the VL domain comprises an amino acid sequence that is at least 90%, at least 95%, at least 99%, or 100% identical to the sequence of SEQ ID NO: 67. In some embodiments, an anti-CD8 antibody of the present disclosure comprises 1, 2, or 3 heavy chain CDRs of antibody xhCD8v4 (e.g., as shown in Tables 1-3) and/or 1, 2, or 3 light chain CDRs of antibody xhCD8v4 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO: 66 and a VL domain comprising a CDR-L1, CDR-L2, and CDR-L3 from the sequence of SEQ ID NO: 67.

In some embodiments, an anti-CD8 antibody of the present disclosure comprises a VH domain comprising a CDR-H1 comprising the amino acid sequence of SEQ ID NO: 31, a CDR-H2 comprising the amino acid sequence of SEQ ID NO: 32, and a CDR-H3 comprising the amino acid sequence of SEQ ID NO: 33 and a VL domain comprising a CDR-L1 comprising the amino acid sequence of SEQ ID NO: 34, a CDR-L2 comprising the amino acid sequence of SEQ ID NO: 35, and a CDR-L3 comprising the amino acid sequence of SEQ ID NO: 36. In some embodiments, the VH domain comprises an amino acid sequence that is at least 90%, at least 95%, at least 99%, or 100% identical to the sequence of SEQ ID NO: 68 and/or the VL domain comprises an amino acid sequence that is at least 90%, at least 95%, at least 99%, or 100% identical to the sequence of SEQ ID NO: 69. In some embodiments, an anti-CD8 antibody of the present disclosure comprises 1, 2, or 3 heavy chain CDRs of antibody xhCD8v5 (e.g., as shown in Tables 1-3) and/or 1, 2, or 3 light chain CDRs of antibody xhCD8v5 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO: 68 and a VL domain comprising a CDR-L1, CDR-L2, and CDR-L3 from the sequence of SEQ ID NO: 69.

In some embodiments, an anti-CD8 antibody of the present disclosure comprises a VH domain comprising a CDR-H1 comprising the amino acid sequence of SEQ ID NO: 37, a CDR-H2 comprising the amino acid sequence of SEQ ID NO: 38, and a CDR-H3 comprising the amino acid sequence of SEQ ID NO: 39 and a VL domain comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42. In some embodiments, the VH domain comprises an amino acid sequence that is at least 90%, at least 95%, at least 99%, or 100% identical to the sequence of SEQ ID NO: 70 and/or the VL domain comprises an amino acid sequence that is at least 90%, at least 95%, at least 99%, or 100% identical to the sequence of SEQ ID NO: 71. In some embodiments, an anti-CD8 antibody of the present disclosure comprises 1, 2, or 3 heavy chain CDRs of antibody xhCD8v6 (e.g., as shown in Tables 1-3) and/or 1, 2, or 3 light chain CDRs of antibody xhCD8v6 (e.g., as shown in Tables 1-3). In some embodiments, the antibody is human. In some embodiments, an anti-CD8 antibody of the present disclosure comprises a VH domain comprising a CDR-H1, CDR-H2, and CDR-H3 from the sequence of SEQ ID NO: 70 and a VL domain comprising a CDR-L1, CDR-L2, and CDR-L3 from the sequence of SEQ ID NO: 71.

In some embodiments, an anti-CD8 antibody of the present disclosure comprises a VH domain comprising a CDR-H1 comprising the amino acid sequence of SEQ ID NO: 43, a CDR-H2 comprising the amino acid sequence of SEQ ID NO: 44, and a CDR-H3 comprising the amino acid sequence of SEQ ID NO: 45 and a VL domain comprising a CDR-L1 comprising the amino acid sequence of SEQ ID NO: 46, a CDR-L2 comprising the amino acid sequence of SEQ ID NO: 47, and a CDR-L3 comprising the amino acid sequence of SEQ ID NO: 48. In some embodiments, the VH domain comprises an amino acid sequence that is at least 90%, at least 95%, at least 99%, or 100% identical to the sequence of SEQ ID NO: 72 and/or the VL domain comprises an amino acid sequence that is at least 90%, at least 95%, at least 99%, or 100% identical to the sequence of SEQ ID NO: 73. In some embodiments, an anti-CD8 antibody of the present disclosure comprises 1, 2, or 3 heavy chain CDRs of antibody xhCD8v7 (e.g., as shown in Tables 1-3) and/or 1, 2, or 3 light chain CDRs of antibody xhCD8v7 (e.g., as shown in Tables 1-3). In some embodiments, the antibody is human. In some embodiments, an anti-CD8 antibody of the present disclosure comprises a VH domain comprising a CDR-H1, CDR-H2, and CDR-H3 from the sequence of SEQ ID NO: 72 and a VL domain comprising a CDR-L1, CDR-L2, and CDR-L3 from the sequence of SEQ ID NO: 73.

In some embodiments, an anti-CD8 antibody of the present disclosure comprises a VH domain comprising a CDR-H1 comprising the amino acid sequence of X₁X₂AIS, wherein X₁ is S, K, G, N, R, D, T, or G, and wherein X₂ is Y, L, H, or F (SEQ ID NO: 259), a CDR-H2 comprising the amino acid sequence of X₁X₂X₃PX₄X₅X₆X₇X₈X₉YX₁₀QKFX₁₁G, wherein X₁ is G or H, X₂ is I or F, X₃ is I, N, or M, X₄ is G, N, H, S, R, I, or A, X₅ is A, N, H, S, T, F, or Y, X₆ is A, D, or G, X₇ is T, E, K, V, Q, or A, X₈ is A or T, X₉ is N or K, X₁₀ is A or N, and X₁₁ is Q or T (SEQ ID NO: 260), and a CDR-H3 comprising the amino acid sequence of X₁X₂X₃GX₄X₅LFX₆X₇, wherein X₁ is D or A, X₂ is A, G, E, R, Y, K, N, Q, L, or F, X₃ is A, L, P, or Y, X₄ is I or L, X₅ is R, A, Q, or S, X₆ is A or D, and X₇ is D, E, A, or S (SEQ ID NO: 261) and a VL domain comprising a CDR-L1 comprising the amino acid sequence of X₁X₂SX₃X₄IX₅GX₆LN, wherein X₁ is R or G, X₂ is A or T, X₃ is Q or E, X₄ is E, N, T, S, A, K, D, G, R, or Q, X₅ is Y or S, and X₆ is A or V (SEQ ID NO: 262), a CDR-L2 comprising the amino acid sequence of GX₁X₂X₃LX₄X₅, wherein X₁ is A or S, X₂ is T, S, E, Q, or D, X₃ is N, R, A, E, or H, X₄ is Q or A, and X₅ is S or D (SEQ ID NO: 263), and a CDR-L3 comprising the amino acid sequence of QX₁X₂X₃X₄X₅PWT, wherein X₁ is S, N, D, Q, A, or E, X₂ is T, I, or S, X₃ is Y, L, or F, X₄ is D, G, T, E, Q, A, or Y, and X₅ is A, T, R, S, K, or Y (SEQ ID NO: 264). In some embodiments, the VH domain further comprises a FW-1 comprising the sequence QVQLVQSGAEVKKPGSSVKVSCKASGGTFS (SEQ ID NO: 274), a FW-2 comprising the sequence WVRQAPGQGLEWMG (SEQ ID NO: 275), a FW-3 comprising the sequence RVTITADESTSTAYMELSSLRSEDTAVYYCAR (SEQ ID NO: 276), and/or a FW-4 comprising the sequence WGQGTLVTVSS (SEQ ID NO: 277). In some embodiments, the VL domain further comprises a FW-1 comprising the sequence DIQMTQSPSSLSASVGDRVTITC (SEQ ID NO: 289), a FW-2 comprising the sequence WYQQKPGKAPKLLIY (SEQ ID NO: 290), a FW-3 comprising the sequence GVPSRFSGSGSGTDFTLTISSLQPEDFATYYC (SEQ ID NO: 291), and/or a FW-4 comprising the sequence FGGGTKVEIK (SEQ ID NO: 292).

In some embodiments, an anti-CD8 antibody of the present disclosure comprises a VH domain comprising a CDR-H1 comprising the amino acid sequence of SEQ ID NO: 225, a CDR-H2 comprising the amino acid sequence of SEQ ID NO: 226, and a CDR-H3 comprising the amino acid sequence of SEQ ID NO: 227 and a VL domain comprising a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228. In some embodiments, the VH domain comprises an amino acid sequence that is at least 90%, at least 95%, at least 99%, or 100% identical to the sequence of SEQ ID NO: 245 and/or the VL domain comprises an amino acid sequence that is at least 90%, at least 95%, at least 99%, or 100% identical to the sequence of SEQ ID NO: 246. In some embodiments, an anti-CD8 antibody of the present disclosure comprises 1, 2, or 3 heavy chain CDRs of antibody xhCD8v9 (e.g., as shown in Tables 1-3) and/or 1, 2, or 3 light chain CDRs of antibody xhCD8v9 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO: 245 and a VL domain comprising a CDR-L1, CDR-L2, and CDR-L3 from the sequence of SEQ ID NO: 246. In some embodiments, the VH domain further comprises a FW-1 comprising the sequence QVQLVQSGAEVKKPGSSVKVSCKASGGTFS (SEQ ID NO: 274), a FW-2 comprising the sequence WVRQAPGQGLEWMG (SEQ ID NO: 275), a FW-3 comprising the sequence RVTITADESTSTAYMELSSLRSEDTAVYYCAR (SEQ ID NO: 276), and/or a FW-4 comprising the sequence WGQGTLVTVSS (SEQ ID NO: 277). In some embodiments, the VL domain further comprises a FW-1 comprising the sequence DIQMTQSPSSLSASVGDRVTITC (SEQ ID NO: 289), a FW-2 comprising the sequence WYQQKPGKAPKLLIY (SEQ ID NO: 290), a FW-3 comprising the sequence GVPSRFSGSGSGTDFTLTISSLQPEDFATYYC (SEQ ID NO: 291), and/or a FW-4 comprising the sequence FGGGTKVEIK (SEQ ID NO: 292).

In some embodiments, an anti-CD8 antibody of the present disclosure comprises a VH domain comprising a CDR-H1 comprising the amino acid sequence of SEQ ID NO: 225, a CDR-H2 comprising the amino acid sequence of SEQ ID NO: 232, and a CDR-H3 comprising the amino acid sequence of SEQ ID NO: 233 and a VL domain comprising a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236. In some embodiments, the VH domain comprises an amino acid sequence that is at least 90%, at least 95%, at least 99%, or 100% identical to the sequence of SEQ ID NO: 251 and/or the VL domain comprises an amino acid sequence that is at least 90%, at least 95%, at least 99%, or 100% identical to the sequence of SEQ ID NO: 252. In some embodiments, the VH domain comprises the amino acid sequence of SEQ ID NO: 251; and the VL domain comprises the amino acid sequence of SEQ ID NO: 252. In some embodiments, an anti-CD8 antibody of the present disclosure comprises 1, 2, or 3 heavy chain CDRs of antibody xhCD8v12 (e.g., as shown in Tables 1-3) and/or 1, 2, or 3 light chain CDRs of antibody xhCD8v12 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO: 251 and a VL domain comprising a CDR-L1, CDR-L2, and CDR-L3 from the sequence of SEQ ID NO: 252. In some embodiments, the VH domain further comprises a FW-1 comprising the sequence QVQLVQSGAEVKKPGSSVKVSCKASGGTFS (SEQ ID NO: 274), a FW-2 comprising the sequence WVRQAPGQGLEWMG (SEQ ID NO: 275), a FW-3 comprising the sequence RVTITADESTSTAYMELSSLRSEDTAVYYCAR (SEQ ID NO: 276), and/or a FW-4 comprising the sequence WGQGTLVTVSS (SEQ ID NO: 277). In some embodiments, the VL domain further comprises a FW-1 comprising the sequence DIQMTQSPSSLSASVGDRVTITC (SEQ ID NO: 289), a FW-2 comprising the sequence WYQQKPGKAPKLLIY (SEQ ID NO: 290), a FW-3 comprising the sequence GVPSRFSGSGSGTDFTLTISSLQPEDFATYYC (SEQ ID NO: 291), and/or a FW-4 comprising the sequence FGGGTKVEIK (SEQ ID NO: 292).

In some embodiments, an anti-CD8 antibody of the present disclosure comprises a VH domain comprising a CDR-H1 comprising the amino acid sequence of SEQ ID NO: 225, a CDR-H2 comprising the amino acid sequence of SEQ ID NO: 232, and a CDR-H3 comprising the amino acid sequence of SEQ ID NO: 233 and a VL domain comprising a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228. In some embodiments, the VH domain comprises an amino acid sequence that is at least 90%, at least 95%, at least 99%, or 100% identical to the sequence of SEQ ID NO: 253 and/or the VL domain comprises an amino acid sequence that is at least 90%, at least 95%, at least 99%, or 100% identical to the sequence of SEQ ID NO: 254. In some embodiments, an anti-CD8 antibody of the present disclosure comprises 1, 2, or 3 heavy chain CDRs of antibody xhCD8v13 (e.g., as shown in Tables 1-3) and/or 1, 2, or 3 light chain CDRs of antibody xhCD8v13 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO: 253 and a VL domain comprising a CDR-L1, CDR-L2, and CDR-L3 from the sequence of SEQ ID NO: 254. In some embodiments, the VH domain further comprises a FW-1 comprising the sequence QVQLVQSGAEVKKPGSSVKVSCKASGGTFS (SEQ ID NO: 274), a FW-2 comprising the sequence WVRQAPGQGLEWMG (SEQ ID NO: 275), a FW-3 comprising the sequence RVTITADESTSTAYMELSSLRSEDTAVYYCAR (SEQ ID NO: 276), and/or a FW-4 comprising the sequence WGQGTLVTVSS (SEQ ID NO: 277). In some embodiments, the VL domain further comprises a FW-1 comprising the sequence DIQMTQSPSSLSASVGDRVTITC (SEQ ID NO: 289), a FW-2 comprising the sequence WYQQKPGKAPKLLIY (SEQ ID NO: 290), a FW-3 comprising the sequence GVPSRFSGSGSGTDFTLTISSLQPEDFATYYC (SEQ ID NO: 291), and/or a FW-4 comprising the sequence FGGGTKVEIK (SEQ ID NO: 292).

In some embodiments, an anti-CD8 antibody of the present disclosure comprises a VH domain comprising a CDR-H1 comprising the amino acid sequence of X₁YX₂MS, wherein X₁ is S, D, E, A, or Q and X₂ is A, G, or T (SEQ ID NO: 268), a CDR-H2 comprising the amino acid sequence of DIX₁X₂X₃GX₄X₅TX₆YADSVKG, wherein X₁ is T, N, S, Q, E, H, R, or A, X₂ is Y, W, F, or H, X₃ is A, S, Q, E, or T, X₄ is G or E, X₅ is S or I, and X₆ is A or G (SEQ ID NO: 269), and a CDR-H3 comprising the amino acid sequence of X₁X₂X₃YX₄WX₅X₆AX₇DX₈, wherein X₁ is S or A, X₂ is N, H, A, D, L, Q, Y, or R, X₃ is A, N, S, or G, X₄ is A, V, R, E, or S, X₅ is D or S, X₆ is D, N, Q, E, S, T, or L, X₇ is L, F, or M, and X₈ is I, Y, or V (SEQ ID NO: 270) and a VL domain comprising a CDR-L1 comprising the amino acid sequence of RASQSVSSNLA (SEQ ID NO: 40), a CDR-L2 comprising the amino acid sequence of GASSRAT (SEQ ID NO: 41), and a CDR-L3 comprising the amino acid sequence of QQYGSSPPVT (SEQ ID NO: 42). In some embodiments, the VH domain further comprises a FW-1 comprising the sequence EVQLVESGGGLVQPGGSLRLSCAASGFTFS (SEQ ID NO: 281), a FW-2 comprising the sequence WVRQAPGKGLEWVS (SEQ ID NO: 282), a FW-3 comprising the sequence RFTISRDNAKNSLYLQMNSLRAEDTAVYYCAR (SEQ ID NO: 283), and/or a FW-4 comprising the sequence WGQGTMVTVSS (SEQ ID NO: 284) or WGQGTLVTVSS (SEQ ID NO: 285). In some embodiments, the VL domain further comprises a FW-1 comprising the sequence EIVLTQSPGTLSLSPGERATLSC (SEQ ID NO: 293), a FW-2 comprising the sequence WYQQKPGQAPRLLIY (SEQ ID NO: 294), a FW-3 comprising the sequence GIPDRFSGSGSGTDFTLTISRLEPEDFAVYYC (SEQ ID NO: 295), and/or a FW-4 comprising the sequence FGQGTKVEIK (SEQ ID NO: 296).

In some embodiments, an anti-CD8 antibody of the present disclosure comprises a VH domain comprising a CDR-H1 comprising the amino acid sequence of SEQ ID NO: 229, a CDR-H2 comprising the amino acid sequence of SEQ ID NO: 230, and a CDR-H3 comprising the amino acid sequence of SEQ ID NO: 231 and a VL domain comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42. In some embodiments, the VH domain comprises an amino acid sequence that is at least 90%, at least 95%, at least 99%, or 100% identical to the sequence of SEQ ID NO: 247 and/or the VL domain comprises an amino acid sequence that is at least 90%, at least 95%, at least 99%, or 100% identical to the sequence of SEQ ID NO: 248. In some embodiments, an anti-CD8 antibody of the present disclosure comprises 1, 2, or 3 heavy chain CDRs of antibody xhCD8v10 (e.g., as shown in Tables 1-3) and/or 1, 2, or 3 light chain CDRs of antibody xhCD8v10 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO: 247 and a VL domain comprising a CDR-L1, CDR-L2, and CDR-L3 from the sequence of SEQ ID NO: 248. In some embodiments, the VH domain further comprises a FW-1 comprising the sequence EVQLVESGGGLVQPGGSLRLSCAASGFTFS (SEQ ID NO: 281), a FW-2 comprising the sequence WVRQAPGKGLEWVS (SEQ ID NO: 282), a FW-3 comprising the sequence RFTISRDNAKNSLYLQMNSLRAEDTAVYYCAR (SEQ ID NO: 283), and/or a FW-4 comprising the sequence WGQGTMVTVSS (SEQ ID NO: 284) or WGQGTLVTVSS (SEQ ID NO: 285). In some embodiments, the VL domain further comprises a FW-1 comprising the sequence EIVLTQSPGTLSLSPGERATLSC (SEQ ID NO: 293), a FW-2 comprising the sequence WYQQKPGQAPRLLIY (SEQ ID NO: 294), a FW-3 comprising the sequence GIPDRFSGSGSGTDFTLTISRLEPEDFAVYYC (SEQ ID NO: 295), and/or a FW-4 comprising the sequence FGQGTKVEIK (SEQ ID NO: 296).

In some embodiments, an anti-CD8 antibody of the present disclosure comprises a VH domain comprising a CDR-H1 comprising the amino acid sequence of SEQ ID NO: 229, a CDR-H2 comprising the amino acid sequence of SEQ ID NO: 230, and a CDR-H3 comprising the amino acid sequence of SEQ ID NO: 231 and a VL domain comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42. In some embodiments, the VH domain comprises an amino acid sequence that is at least 90%, at least 95%, at least 99%, or 100% identical to the sequence of SEQ ID NO: 249 and/or the VL domain comprises an amino acid sequence that is at least 90%, at least 95%, at least 99%, or 100% identical to the sequence of SEQ ID NO: 250. In some embodiments, the VH domain comprises the amino acid sequence of SEQ ID NO: 249; and the VL domain comprises the amino acid sequence of SEQ ID NO: 250. In some embodiments, an anti-CD8 antibody of the present disclosure comprises 1, 2, or 3 heavy chain CDRs of antibody xhCD8v11 (e.g., as shown in Tables 1-3) and/or 1, 2, or 3 light chain CDRs of antibody xhCD8v11 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO: 249 and a VL domain comprising a CDR-L1, CDR-L2, and CDR-L3 from the sequence of SEQ ID NO: 250. In some embodiments, the VH domain further comprises a FW-1 comprising the sequence EVQLVESGGGLVQPGGSLRLSCAASGFTFS (SEQ ID NO: 281), a FW-2 comprising the sequence WVRQAPGKGLEWVS (SEQ ID NO: 282), a FW-3 comprising the sequence RFTISRDNAKNSLYLQMNSLRAEDTAVYYCAR (SEQ ID NO: 283), and/or a FW-4 comprising the sequence WGQGTMVTVSS (SEQ ID NO: 284) or WGQGTLVTVSS (SEQ ID NO: 285). In some embodiments, the VL domain further comprises a FW-1 comprising the sequence EIVLTQSPGTLSLSPGERATLSC (SEQ ID NO: 293), a FW-2 comprising the sequence WYQQKPGQAPRLLIY (SEQ ID NO: 294), a FW-3 comprising the sequence GIPDRFSGSGSGTDFTLTISRLEPEDFAVYYC (SEQ ID NO: 295), and/or a FW-4 comprising the sequence FGQGTKVEIK (SEQ ID NO: 296).

In some embodiments, an anti-CD8 antibody of the present disclosure comprises a VH domain comprising a CDR-H1 comprising the amino acid sequence of SEQ ID NO: 229, a CDR-H2 comprising the amino acid sequence of SEQ ID NO: 237, and a CDR-H3 comprising the amino acid sequence of SEQ ID NO: 231 and a VL domain comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42. In some embodiments, the VH domain comprises an amino acid sequence that is at least 90%, at least 95%, at least 99%, or 100% identical to the sequence of SEQ ID NO: 255 and/or the VL domain comprises an amino acid sequence that is at least 90%, at least 95%, at least 99%, or 100% identical to the sequence of SEQ ID NO: 256. In some embodiments, an anti-CD8 antibody of the present disclosure comprises 1, 2, or 3 heavy chain CDRs of antibody xhCD8v14 (e.g., as shown in Tables 1-3) and/or 1, 2, or 3 light chain CDRs of antibody xhCD8v14 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO: 255 and a VL domain comprising a CDR-L1, CDR-L2, and CDR-L3 from the sequence of SEQ ID NO: 256. In some embodiments, the VH domain further comprises a FW-1 comprising the sequence EVQLVESGGGLVQPGGSLRLSCAASGFTFS (SEQ ID NO: 281), a FW-2 comprising the sequence WVRQAPGKGLEWVS (SEQ ID NO: 282), a FW-3 comprising the sequence RFTISRDNAKNSLYLQMNSLRAEDTAVYYCAR (SEQ ID NO: 283), and/or a FW-4 comprising the sequence WGQGTMVTVSS (SEQ ID NO: 284) or WGQGTLVTVSS (SEQ ID NO: 285). In some embodiments, the VL domain further comprises a FW-1 comprising the sequence EIVLTQSPGTLSLSPGERATLSC (SEQ ID NO: 293), a FW-2 comprising the sequence WYQQKPGQAPRLLIY (SEQ ID NO: 294), a FW-3 comprising the sequence GIPDRFSGSGSGTDFTLTISRLEPEDFAVYYC (SEQ ID NO: 295), and/or a FW-4 comprising the sequence FGQGTKVEIK (SEQ ID NO: 296).

In some embodiments, an anti-CD8 antibody of the present disclosure comprises a VH domain comprising a CDR-H1 comprising the amino acid sequence of SEQ ID NO: 229, a CDR-H2 comprising the amino acid sequence of SEQ ID NO: 237, and a CDR-H3 comprising the amino acid sequence of SEQ ID NO: 231 and a VL domain comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42. In some embodiments, the VH domain comprises an amino acid sequence that is at least 90%, at least 95%, at least 99%, or 100% identical to the sequence of SEQ ID NO: 257 and/or the VL domain comprises an amino acid sequence that is at least 90%, at least 95%, at least 99%, or 100% identical to the sequence of SEQ ID NO: 258. In some embodiments, an anti-CD8 antibody of the present disclosure comprises 1, 2, or 3 heavy chain CDRs of antibody xhCD8v15 (e.g., as shown in Tables 1-3) and/or 1, 2, or 3 light chain CDRs of antibody xhCD8v15 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO: 257 and a VL domain comprising a CDR-L1, CDR-L2, and CDR-L3 from the sequence of SEQ ID NO: 258. In some embodiments, the VH domain further comprises a FW-1 comprising the sequence EVQLVESGGGLVQPGGSLRLSCAASGFTFS (SEQ ID NO: 281), a FW-2 comprising the sequence WVRQAPGKGLEWVS (SEQ ID NO: 282), a FW-3 comprising the sequence RFTISRDNAKNSLYLQMNSLRAEDTAVYYCAR (SEQ ID NO: 283), and/or a FW-4 comprising the sequence WGQGTMVTVSS (SEQ ID NO: 284) or WGQGTLVTVSS (SEQ ID NO: 285). In some embodiments, the VL domain further comprises a FW-1 comprising the sequence EIVLTQSPGTLSLSPGERATLSC (SEQ ID NO: 293), a FW-2 comprising the sequence WYQQKPGQAPRLLIY (SEQ ID NO: 294), a FW-3 comprising the sequence GIPDRFSGSGSGTDFTLTISRLEPEDFAVYYC (SEQ ID NO: 295), and/or a FW-4 comprising the sequence FGQGTKVEIK (SEQ ID NO: 296).

In some embodiments, an anti-CD8 antibody of the present disclosure comprises a VH domain comprising a CDR-H1 comprising the amino acid sequence of SEQ ID NO: 49, a CDR-H2 comprising the amino acid sequence of SEQ ID NO: 50, and a CDR-H3 comprising the amino acid sequence of SEQ ID NO: 3 and a VL domain comprising a CDR-L1 comprising the amino acid sequence of SEQ ID NO: 4, a CDR-L2 comprising the amino acid sequence of SEQ ID NO: 5, and a CDR-L3 comprising the amino acid sequence of SEQ ID NO: 6. In some embodiments, an anti-CD8 antibody of the present disclosure comprises a VH domain comprising a CDR-H1 comprising the amino acid sequence of SEQ ID NO: 51, a CDR-H2 comprising the amino acid sequence of SEQ ID NO: 52, and a CDR-H3 comprising the amino acid sequence of SEQ ID NO: 15 and a VL domain comprising a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 18. In some embodiments, an anti-CD8 antibody of the present disclosure comprises a VH domain comprising a CDR-H1 comprising the amino acid sequence of SEQ ID NO: 53, a CDR-H2 comprising the amino acid sequence of SEQ ID NO: 52, and a CDR-H3 comprising the amino acid sequence of SEQ ID NO: 21 and a VL domain comprising a CDR-L1 comprising the amino acid sequence of SEQ ID NO: 22, a CDR-L2 comprising the amino acid sequence of SEQ ID NO: 23, and a CDR-L3 comprising the amino acid sequence of SEQ ID NO: 24. In some embodiments, an anti-CD8 antibody of the present disclosure comprises a VH domain comprising a CDR-H1 comprising the amino acid sequence of SEQ ID NO: 49, a CDR-H2 comprising the amino acid sequence of SEQ ID NO: 52, and a CDR-H3 comprising the amino acid sequence of SEQ ID NO: 27 and a VL domain comprising a CDR-L1 comprising the amino acid sequence of SEQ ID NO: 28, a CDR-L2 comprising the amino acid sequence of SEQ ID NO: 29, and a CDR-L3 comprising the amino acid sequence of SEQ ID NO: 30. In some embodiments, an anti-CD8 antibody of the present disclosure comprises a VH domain comprising a CDR-H1 comprising the amino acid sequence of SEQ ID NO: 54, a CDR-H2 comprising the amino acid sequence of SEQ ID NO: 52, and a CDR-H3 comprising the amino acid sequence of SEQ ID NO: 33 and a VL domain comprising a CDR-L1 comprising the amino acid sequence of SEQ ID NO: 34, a CDR-L2 comprising the amino acid sequence of SEQ ID NO: 35, and a CDR-L3 comprising the amino acid sequence of SEQ ID NO: 36. In some embodiments, an anti-CD8 antibody of the present disclosure comprises a VH domain comprising a CDR-H1 comprising the amino acid sequence of SEQ ID NO: 55, a CDR-H2 comprising the amino acid sequence of SEQ ID NO: 56, and a CDR-H3 comprising the amino acid sequence of SEQ ID NO: 39 and a VL domain comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42. In some embodiments, an anti-CD8 antibody of the present disclosure comprises a VH domain comprising a CDR-H1 comprising the amino acid sequence of SEQ ID NO: 55, a CDR-H2 comprising the amino acid sequence of SEQ ID NO: 57, and a CDR-H3 comprising the amino acid sequence of SEQ ID NO: 45 and a VL domain comprising a CDR-L1 comprising the amino acid sequence of SEQ ID NO: 46, a CDR-L2 comprising the amino acid sequence of SEQ ID NO: 47, and a CDR-L3 comprising the amino acid sequence of SEQ ID NO: 48. In some embodiments, an anti-CD8 antibody of the present disclosure comprises a VH domain comprising a CDR-H1 comprising the amino acid sequence of SEQ ID NO: 183, a CDR-H2 comprising the amino acid sequence of SEQ ID NO: 184, and a CDR-H3 comprising the amino acid sequence of SEQ ID NO: 179 and a VL domain comprising a CDR-L1 comprising the amino acid sequence of SEQ ID NO: 180, a CDR-L2 comprising the amino acid sequence of SEQ ID NO: 181, and a CDR-L3 comprising the amino acid sequence of SEQ ID NO: 182.

In some embodiments, an anti-CD8 antibody of the present disclosure comprises a VH domain comprising a CDR-H1 comprising the amino acid sequence of GX₁X₂FX₃X₄X₅, wherein X₁ is G, Y, S, or A, X₂ is T, S, G, R, N, or H, X₃ is S, T, R, H, Y, G, or P, X₄ is S, K, G, N, R, D, T, or G, and X₅ is Y, L, H, or F (SEQ ID NO: 265), a CDR-H2 comprising the amino acid sequence of X₁PX₂X₃X₄X₅, wherein X₁ is I, N, or M, X₂ is G, N, H, S, R, I, or A, X₃ is A, N, H, S, T, F, or Y, X₄ is A, D, or G, and X₅ is T, E, K, V, Q, or A (SEQ ID NO: 266), and a CDR-H3 comprising the amino acid sequence of X₁X₂X₃GX₄X₅LFX₆X₇, wherein X₁ is D or A, X₂ is A, G, E, R, Y, K, N, Q, L, or F, X₃ is A, L, P, or Y, X₄ is I or L, X₅ is R, A, Q, or S, X₆ is A or D, and X₇ is D, E, A, or S (SEQ ID NO: 267) and a VL domain comprising a CDR-L1 comprising the amino acid sequence of X₁X₂SX₃X₄IX₅GX₆LN, wherein X₁ is R or G, X₂ is A or T, X₃ is Q or E, X₄ is E, N, T, S, A, K, D, G, R, or Q, X₅ is Y or S, and X₆ is A or V (SEQ ID NO: 262), a CDR-L2 comprising the amino acid sequence of GX₁X₂X₃LX₄X₅, wherein X₁ is A or S, X₂ is T, S, E, Q, or D, X₃ is N, R, A, E, or H, X₄ is Q or A, and X₅ is S or D (SEQ ID NO: 263), and a CDR-L3 comprising the amino acid sequence of QX₁X₂X₃X₄X₅PWT, wherein X₁ is S, N, D, Q, A, or E, X₂ is T, I, or S, X₃ is Y, L, or F, X₄ is D, G, T, E, Q, A, or Y, and X₅ is A, T, R, S, K, or Y (SEQ ID NO: 264). In some embodiments, the VH domain further comprises a FW-1 comprising the sequence QVQLVQSGAEVKKPGSSVKVSCKAS (SEQ ID NO: 278), a FW-2 comprising the sequence AISWVRQAPGQGLEWMGGI (SEQ ID NO: 279), a FW-3 comprising the sequence ANYAQKFQGRVTITADESTSTAYMELSSLRSEDTAVYYCAR (SEQ ID NO: 280), and/or a FW-4 comprising the sequence WGQGTLVTVSS (SEQ ID NO: 277). In some embodiments, the VL domain further comprises a FW-1 comprising the sequence DIQMTQSPSSLSASVGDRVTITC (SEQ ID NO: 289), a FW-2 comprising the sequence WYQQKPGKAPKLLIY (SEQ ID NO: 290), a FW-3 comprising the sequence GVPSRFSGSGSGTDFTLTISSLQPEDFATYYC (SEQ ID NO: 291), and/or a FW-4 comprising the sequence FGGGTKVEIK (SEQ ID NO: 292).

In some embodiments, an anti-CD8 antibody of the present disclosure comprises a VH domain comprising a CDR-H1 comprising the amino acid sequence of SEQ ID NO: 238, a CDR-H2 comprising the amino acid sequence of SEQ ID NO: 239, and a CDR-H3 comprising the amino acid sequence of SEQ ID NO: 233 and a VL domain comprising a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228. In some embodiments, the VH domain further comprises a FW-1 comprising the sequence QVQLVQSGAEVKKPGSSVKVSCKAS (SEQ ID NO: 278), a FW-2 comprising the sequence AISWVRQAPGQGLEWMGGI (SEQ ID NO: 279), a FW-3 comprising the sequence ANYAQKFQGRVTITADESTSTAYMELSSLRSEDTAVYYCAR (SEQ ID NO: 280), and/or a FW-4 comprising the sequence WGQGTLVTVSS (SEQ ID NO: 277). In some embodiments, the VL domain further comprises a FW-1 comprising the sequence DIQMTQSPSSLSASVGDRVTITC (SEQ ID NO: 289), a FW-2 comprising the sequence WYQQKPGKAPKLLIY (SEQ ID NO: 290), a FW-3 comprising the sequence GVPSRFSGSGSGTDFTLTISSLQPEDFATYYC (SEQ ID NO: 291), and/or a FW-4 comprising the sequence FGGGTKVEIK (SEQ ID NO: 292).

In some embodiments, an anti-CD8 antibody of the present disclosure comprises a VH domain comprising a CDR-H1 comprising the amino acid sequence of SEQ ID NO: 238, a CDR-H2 comprising the amino acid sequence of SEQ ID NO: 243, and a CDR-H3 comprising the amino acid sequence of SEQ ID NO: 233 and a VL domain comprising a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236. In some embodiments, the VH domain further comprises a FW-1 comprising the sequence QVQLVQSGAEVKKPGSSVKVSCKAS (SEQ ID NO: 278), a FW-2 comprising the sequence AISWVRQAPGQGLEWMGGI (SEQ ID NO: 279), a FW-3 comprising the sequence ANYAQKFQGRVTITADESTSTAYMELSSLRSEDTAVYYCAR (SEQ ID NO: 280), and/or a FW-4 comprising the sequence WGQGTLVTVSS (SEQ ID NO: 277). In some embodiments, the VL domain further comprises a FW-1 comprising the sequence DIQMTQSPSSLSASVGDRVTITC (SEQ ID NO: 289), a FW-2 comprising the sequence WYQQKPGKAPKLLIY (SEQ ID NO: 290), a FW-3 comprising the sequence GVPSRFSGSGSGTDFTLTISSLQPEDFATYYC (SEQ ID NO: 291), and/or a FW-4 comprising the sequence FGGGTKVEIK (SEQ ID NO: 292).

In some embodiments, an anti-CD8 antibody of the present disclosure comprises a VH domain comprising a CDR-H1 comprising the amino acid sequence of SEQ ID NO: 238, a CDR-H2 comprising the amino acid sequence of SEQ ID NO: 243, and a CDR-H3 comprising the amino acid sequence of SEQ ID NO: 233 and a VL domain comprising a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228. In some embodiments, the VH domain further comprises a FW-1 comprising the sequence QVQLVQSGAEVKKPGSSVKVSCKAS (SEQ ID NO: 278), a FW-2 comprising the sequence AISWVRQAPGQGLEWMGGI (SEQ ID NO: 279), a FW-3 comprising the sequence ANYAQKFQGRVTITADESTSTAYMELSSLRSEDTAVYYCAR (SEQ ID NO: 280), and/or a FW-4 comprising the sequence WGQGTLVTVSS (SEQ ID NO: 277). In some embodiments, the VL domain further comprises a FW-1 comprising the sequence DIQMTQSPSSLSASVGDRVTITC (SEQ ID NO: 289), a FW-2 comprising the sequence WYQQKPGKAPKLLIY (SEQ ID NO: 290), a FW-3 comprising the sequence GVPSRFSGSGSGTDFTLTISSLQPEDFATYYC (SEQ ID NO: 291), and/or a FW-4 comprising the sequence FGGGTKVEIK (SEQ ID NO: 292).

In some embodiments, an anti-CD8 antibody of the present disclosure comprises a VH domain comprising a CDR-H1 comprising the amino acid sequence of GFTFX₁X₂Y, wherein X₁ is S, D, E, Q, S, or A and X₂ is S, D, E, A, or Q (SEQ ID NO: 271), a CDR-H2 comprising the amino acid sequence of X₁X₂X₃GX₄X₅, wherein X₁ is T, N, S, Q, E, H, R or A, X₂ is Y, W, F, or H, X₃ is A, S, Q, E, or T, X₄ is G or E, and X₅ is S or I (SEQ ID NO: 272), and a CDR-H3 comprising the amino acid sequence of X₁X₂X₃YX₄WX₅X₆AX₇DX₈, wherein X₁ is S or A, X₂ is N, H, A, D, L, Q, Y, or R, X₃ is A, N, S, or G, X₄ is A, V, R, E, or S, X₅ is D or S, X₆ is D, N, Q, E, S, T, or L, X₇ is L, F, or M, and X₈ is I, Y, or V (SEQ ID NO: 273) and a VL domain comprising a CDR-L1 comprising the amino acid sequence of RASQSVSSNLA (SEQ ID NO: 40), a CDR-L2 comprising the amino acid sequence of GASSRAT (SEQ ID NO: 41), and a CDR-L3 comprising the amino acid sequence of QQYGSSPPVT (SEQ ID NO: 42). In some embodiments, the VH domain further comprises a FW-1 comprising the sequence EVQLVESGGGLVQPGGSLRLSCAAS (SEQ ID NO: 286), a FW-2 comprising the sequence AMSWVRQAPGKGLEWVSDI (SEQ ID NO: 287), a FW-3 comprising the sequence TAYADSVKGRFTISRDNAKNSLYLQMNSLRAEDTAVYYCAR (SEQ ID NO: 288), and/or a FW-4 comprising the sequence WGQGTMVTVSS (SEQ ID NO: 284) or WGQGTLVTVSS (SEQ ID NO: 285). In some embodiments, the VL domain further comprises a FW-1 comprising the sequence EIVLTQSPGTLSLSPGERATLSC (SEQ ID NO: 293), a FW-2 comprising the sequence WYQQKPGQAPRLLIY (SEQ ID NO: 294), a FW-3 comprising the sequence GIPDRFSGSGSGTDFTLTISRLEPEDFAVYYC (SEQ ID NO: 295), and/or a FW-4 comprising the sequence FGQGTKVEIK (SEQ ID NO: 296).

In some embodiments, an anti-CD8 antibody of the present disclosure comprises a VH domain comprising a CDR-H1 comprising the amino acid sequence of SEQ ID NO: 240, a CDR-H2 comprising the amino acid sequence of SEQ ID NO: 241, and a CDR-H3 comprising the amino acid sequence of SEQ ID NO: 242 and a VL domain comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42. In some embodiments, the VH domain further comprises a FW-1 comprising the sequence EVQLVESGGGLVQPGGSLRLSCAAS (SEQ ID NO: 286), a FW-2 comprising the sequence AMSWVRQAPGKGLEWVSDI (SEQ ID NO: 287), a FW-3 comprising the sequence TAYADSVKGRFTISRDNAKNSLYLQMNSLRAEDTAVYYCAR (SEQ ID NO: 288), and/or a FW-4 comprising the sequence WGQGTMVTVSS (SEQ ID NO: 284) or WGQGTLVTVSS (SEQ ID NO: 285). In some embodiments, the VL domain further comprises a FW-1 comprising the sequence EIVLTQSPGTLSLSPGERATLSC (SEQ ID NO: 293), a FW-2 comprising the sequence WYQQKPGQAPRLLIY (SEQ ID NO: 294), a FW-3 comprising the sequence GIPDRFSGSGSGTDFTLTISRLEPEDFAVYYC (SEQ ID NO: 295), and/or a FW-4 comprising the sequence FGQGTKVEIK (SEQ ID NO: 296).

In some embodiments, an anti-CD8 antibody of the present disclosure comprises a VH domain comprising a CDR-H1 comprising the amino acid sequence of SEQ ID NO: 240, a CDR-H2 comprising the amino acid sequence of SEQ ID NO: 244, and a CDR-H3 comprising the amino acid sequence of SEQ ID NO: 242 and a VL domain comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42. In some embodiments, the VH domain further comprises a FW-1 comprising the sequence EVQLVESGGGLVQPGGSLRLSCAAS (SEQ ID NO: 286), a FW-2 comprising the sequence AMSWVRQAPGKGLEWVSDI (SEQ ID NO: 287), a FW-3 comprising the sequence TAYADSVKGRFTISRDNAKNSLYLQMNSLRAEDTAVYYCAR (SEQ ID NO: 288), and/or a FW-4 comprising the sequence WGQGTMVTVSS (SEQ ID NO: 284) or WGQGTLVTVSS (SEQ ID NO: 285). In some embodiments, the VL domain further comprises a FW-1 comprising the sequence EIVLTQSPGTLSLSPGERATLSC (SEQ ID NO: 293), a FW-2 comprising the sequence WYQQKPGQAPRLLIY (SEQ ID NO: 294), a FW-3 comprising the sequence GIPDRFSGSGSGTDFTLTISRLEPEDFAVYYC (SEQ ID NO: 295), and/or a FW-4 comprising the sequence FGQGTKVEIK (SEQ ID NO: 296). In some embodiments, the present disclosure provides an anti-CD8 antibody comprising a VH domain comprising CDR-H1, CDR-H2, and CDR-H3 sequences of a single antibody listed in Table 1 and a VL domain comprising CDR-L1, CDR-L2, and CDR-L3 sequences of the single antibody listed in Table 1. For example, the anti-CD8 antibody comprises the six CDRs of antibody xhCD8v1, xhCD8v1.1, xhCD8v2, xhCD8v3, xhCD8v4, xhCD8v5, xhCD8v6, xhCD8v7, xhCD8v8, xhCD8v9, xhCD8v10, xhCD8v11, xhCD8v12, xhCD8v13, xhCD8v14, xhCD8v15, V9 family, or V11 family shown in Table 1. In some embodiments, the present disclosure provides an anti-CD8 antibody comprising a VH domain comprising CDR-H1, CDR-H2, and CDR-H3 sequences of a single antibody listed in Table 2 and a VL domain comprising CDR-L1, CDR-L2, and CDR-L3 sequences of the single antibody listed in Table 2. For example, the anti-CD8 antibody comprises the six CDRs of antibody xhCD8v1, xhCD8v1.1, xhCD8v2, xhCD8v3, xhCD8v4, xhCD8v5, xhCD8v6, xhCD8v7, xhCD8v8, xhCD8v9, xhCD8v10, xhCD8v11, xhCD8v12, xhCD8v13, xhCD8v14, xhCD8v15, V9 family, or V11 family shown in Table 2. In some embodiments, the present disclosure provides a fusion protein comprising an anti-CD8 antibody comprising a VH domain comprising CDR-H1, CDR-H2, and CDR-H3 sequences of the single antibody listed in Table 1 and a VL domain comprising CDR-L1, CDR-L2, and CDR-L3 sequences of a single antibody listed in Table 1. For example, the anti-CD8 antibody of the fusion protein comprises the six CDRs of antibody xhCD8v1, xhCD8v1.1, xhCD8v2, xhCD8v3, xhCD8v4, xhCD8v5, xhCD8v6, xhCD8v7, xhCD8v8, xhCD8v9, xhCD8v10, xhCD8v11, xhCD8v12, xhCD8v13, xhCD8v14, xhCD8v15, V9 family, or V11 family shown in Table 1. In some embodiments, the present disclosure provides a fusion protein comprising an anti-CD8 antibody comprising a VH domain comprising CDR-H1, CDR-H2, and CDR-H3 sequences of a single antibody listed in Table 2 and a VL domain comprising CDR-L1, CDR-L2, and CDR-L3 sequences of the single antibody listed in Table 2. For example, the anti-CD8 antibody of the fusion protein comprises the six CDRs of antibody xhCD8v1, xhCD8v1.1, xhCD8v2, xhCD8v3, xhCD8v4, xhCD8v5, xhCD8v6, xhCD8v7, xhCD8v8, xhCD8v9, xhCD8v10, xhCD8v11, xhCD8v12, xhCD8v13, xhCD8v14, xhCD8v15, V9 family, or V11 family shown in Table 2. In some embodiments, the present disclosure provides an anti-CD8 antibody comprising a VH domain comprising CDR-H1, CDR-H2, and CDR-H3 sequences of a VH domain listed in Table 3 and a VL domain comprising CDR-L1, CDR-L2, and CDR-L3 sequences of a VL domain listed in Table 3 (in some embodiments, the VH and VL domains are from the same single antibody listed in Table 3). For example, the anti-CD8 antibody comprises the VH and VL of antibody xhCD8v1, xhCD8v1.1, xhCD8v2, xhCD8v3, xhCD8v4, xhCD8v5, xhCD8v6, xhCD8v7, xhCD8v8, xhCD8v9, xhCD8v10, xhCD8v11, xhCD8v12, xhCD8v13, xhCD8v14, or xhCD8v15 shown in Table 3. In some embodiments, the present disclosure provides a fusion protein comprising an anti-CD8 antibody comprising a VH domain comprising CDR-H1, CDR-H2, and CDR-H3 sequences of a VH domain listed in Table 3 and a VL domain comprising CDR-L1, CDR-L2, and CDR-L3 sequences of a VL domain listed in Table 3 (in some embodiments, the VH and VL domains are from the same single antibody listed in Table 3). In some embodiments, the present disclosure provides an anti-CD8 antibody comprising a VH domain sequence and a VL domain sequence for a single antibody as listed in Table 3. In some embodiments, the present disclosure provides a fusion protein comprising an anti-CD8 antibody comprising a VH domain sequence and a VL domain sequence for a single antibody as listed in Table 3. For example, the anti-CD8 antibody of the fusion protein comprises the VH and VL of antibody xhCD8v1, xhCD8v1.1, xhCD8v2, xhCD8v3, xhCD8v4, xhCD8v5, xhCD8v6, xhCD8v7, xhCD8v8, xhCD8v9, xhCD8v10, xhCD8v11, xhCD8v12, xhCD8v13, xhCD8v14, or xhCD8v15 shown in Table 3.

TABLE 1
Anti-CD8 antibody CDRs (Kabat)

| Name | CDR-H1 | CDR-H2 | CDR-H3 | CDR-L1 | CDR-L2 | CDR-L3 |
|---|---|---|---|---|---|---|
| xhCD8v1 | KYTMH | HFNPNND | DGLGLRL | GASENIY | GATNLAD | QNILDTP |
| | (SEQ ID | ETKYNQK | FAD | GALN | (SEQ ID | WT |
| | NO: 1) | FTG | (SEQ ID | (SEQ ID | NO: 5) | (SEQ ID |
| | | (SEQ ID | NO: 3) | NO: 4) | | NO: 6) |
| | | NO: 2) | | | | |
| xhCD8v1.1 | KYAIS | HFNPNND | DGLGLRL | RASENIY | GATNLAD | QNILDTP |
| | (SEQ ID | ETKYNQK | FAD | GALN | (SEQ ID | WT |
| | NO: 7) | FQG | (SEQ ID | (SEQ ID | NO: 11) | (SEQ ID |
| | | (SEQ ID | NO: 9) | NO: 10) | | NO: 12) |
| | | NO: 8) | | | | |
| xhCD8v2 | NFAIS | GIIPGHAK | DGLGIRL | RASQEIY | GATNLQS | QDIYDAP |
| | (SEQ ID | ANYAQK | FAD | GALN | (SEQ ID | WT |
| | NO: 13) | FQG | (SEQ ID | (SEQ ID | NO: 17) | (SEQ ID |
| | | (SEQ ID | NO: 15) | NO: 16) | | NO: 18) |
| | | NO: 14) | | | | |
| xhCD8v3 | KFAIS | GIIPGHAK | DGLGIRL | RASQEIY | GATNLQS | QDIYDAP |
| | (SEQ ID | ANYAQK | FAD | GALN | (SEQ ID | WT |
| | NO: 19) | FQG | (SEQ ID | (SEQ ID | NO: 23) | (SEQ ID |
| | | (SEQ ID | NO: 21) | NO: 22) | | NO: 24) |
| | | NO: 20) | | | | |
| xhCD8v4 | KYAIS | GIIPGHAK | DGLGIRL | RASQKIY | GATNLQS | QNTYDTP |
| | (SEQ ID | ANYAQK | FAD | GALN | (SEQ ID | WT |
| | NO: 25) | FQG | (SEQ ID | (SEQ ID | NO: 29) | (SEQ ID |
| | | (SEQ ID | NO: 27) | NO: 28) | | NO: 30) |
| | | NO: 26) | | | | |
| xhCD8v5 | GHAIS | GIIPGHAK | DGLGIRL | RASQKIY | GATNLQS | QNTYDTP |
| | (SEQ ID | ANYAQK | FAD | GALN | (SEQ ID | WT |
| | NO: 31) | FQG | (SEQ ID | (SEQ ID | NO: 35) | (SEQ ID |
| | | (SEQ ID | NO: 33) | NO: 34) | | NO: 36) |
| | | NO: 32) | | | | |
| xhCD8v6 | DYGMS | DINWSGE | SNSYRW | RASQSVS | GASSRAT | QQYGSSP |
| | (SEQ ID | ITAYADS | DDALDI | SNLA | (SEQ ID | PVT |
| | NO: 37) | VKG | (SEQ ID | (SEQ ID | NO: 41) | (SEQ ID |
| | | (SEQ ID | NO: 39) | NO: 40) | | NO: 42) |
| | | NO: 38) | | | | |
| xhCD8v7 | DYAMH | VISYDGS | DRIGWYD | RASHSVG | DASNRAT | QQRSNW |
| | (SEQ ID | NKYYAD | YDAFDI | SNLA | (SEQ ID | PPT |
| | NO: 43) | SVKG | (SEQ ID | (SEQ ID | NO: 47) | (SEQ ID |
| | | (SEQ ID | NO: 45) | NO: 46) | | NO: 48) |
| | | NO: 44) | | | | |
| xhCD8v8 | SYWMN | QIYPGDG | SGAAFSS | RASENIY | AATNLAD | QHFWGTP |
| | (SEQ ID | DTNYNG | YYAMDY | SNLA | (SEQ ID | WT (SEQ |
| | NO: 177) | KFKG | (SEQ ID | (SEQ ID | NO: 181) | ID NO: |
| | | (SEQ ID | NO: 179) | NO: 180) | | 182) |
| | | NO: 178) | | | | |
| xhCD8v9 | SYAIS | GIIPGAAT | DAAGIRL | RASQEIY | GATNLQS | QSTYDAP |
| | (SEQ ID | ANYAQK | FAD | GALN | (SEQ ID | WT (SEQ |
| | NO: 225) | FQG | (SEQ ID | (SEQ ID | NO: 17) | ID NO: |
| | | (SEQ ID | NO: 227) | NO: 16) | | 228) |
| | | NO: 226) | | | | |
| xhCD8v10 | SYAMS | DITYAGG | SNAYAW | RASQSVS | GASSRAT | QQYGSSP |
| | (SEQ ID | STAYADS | DDALDI | SNLA | (SEQ ID | PVT |
| | NO: 229) | VKG (SEQ | (SEQ ID | (SEQ ID | NO: 41) | (SEQ ID |
| | | ID NO: | NO: 231) | NO: 40) | | NO: 42) |
| | | 230) | | | | |
| xhCD8v11 | SYAMS | DITYAGG | SNAYAW | RASQSVS | GASSRAT | QQYGSSP |
| | (SEQ ID | STAYADS | DDALDI | SNLA | (SEQ ID | PVT |
| | NO: 229) | VKG (SEQ | (SEQ ID | (SEQ ID | NO: 41) | (SEQ ID |
| | | ID NO: | NO: 231) | NO: 40) | | NO: 42) |
| | | 230) | | | | |
| xhCD8v12 | SYAIS | GIIPGYAT | DAAGIRL | RASQSIY | GASNLQS | QSTYTAP |
| | (SEQ ID | ANYAQK | FAD (SEQ | GALN | (SEQ ID | WT (SEQ |
| | NO: 225) | FQG (SEQ | ID NO: | (SEQ ID | NO: 235) | ID NO: |
| | | ID NO: | 233) | NO: 234) | | 236) |
| | | 232) | | | | |
| xhCD8v13 | SYAIS | GIIPGYAT | DAAGIRL | RASQEIY | GATNLQS | QSTYDAP |
| | (SEQ ID | ANYAQK | FAD (SEQ | GALN | (SEQ ID | WT (SEQ |
| | NO: 225) | FQG (SEQ | ID NO: | (SEQ ID | NO: 17) | ID NO: |
| | | ID NO: | 233) | NO: 16) | | 228) |
| | | 232) | | | | |
| xhCD8v14 | SYAMS | DISYAGG | SNAYAW | RASQSVS | GASSRAT | QQYGSSP |
| | (SEQ ID | STAYADS | DDALDI | SNLA | (SEQ ID | PVT |
| | NO: 229) | VKG (SEQ | (SEQ ID | (SEQ ID | NO: 41) | (SEQ ID |
| | | ID NO: | NO: 231) | NO: 40) | | NO: 42) |
| | | 237) | | | | |
| xhCD8v15 | SYAMS | DISYAGG | SNAYAW | RASQSVS | GASSRAT | QQYGSSP |
| | (SEQ ID | STAYADS | DDALDI | SNLA | (SEQ ID | PVT |
| | NO: 229) | VKG (SEQ | (SEQ ID | (SEQ ID | NO: 41) | (SEQ ID |
| | | ID NO: | NO: 231) | NO: 40) | | NO: 42) |
| | | 237) | | | | |
| V9 family | X₁X₂AIS | X₁X₂X₃PX₄ | X₁X₂X₃GX | X₁X₂SX₃X₄ | GX₁X₂X₃L | QX₁X₂X₃X |
| | X₁ is S, K, | X₅X₆X₇X₈ | ₄X₅LFX₆X₇ | IX₅GX₆LN | X₄X₅ | ₄X₅PWT |
| | G, N, R, D, | X₉YX₁₀QK | X₁ is D or | X₁ is R or | X₁ is A or | X₁ is S, N, |
| | T, or G | FX₁₁G | A, | G, | S, X₂ is T, | D, Q, A, or |
| | X₂ is Y, L, | X₁ is G or | X₂ is A, G, | X₂ is A or | S, E, Q, or | E, |
| | H, or F | H, X₂ is I or | E, R, Y, K, | T. | D, X₃ is N, | X₂ is T, I, |
| | (SEQ ID | F, | N, Q, L, or | X₃ is Q or | R, A, E, or | or S, |
| | NO: 259) | X₃ is I, N, | F, | E, | H, X₄ is Q | X₃ is Y, L, |
| | | or M, | X₃ is A, L, | X₄ is E, N, | or A, | or F, |
| | | X₄ is G, N, | P, or Y, | T, S, A, K, | X₅ is S or D | X₄ is D, G, |
| | | H, S, R, I, | X₄ is I or L, | D, G, R, or | (SEQ ID | T, E, Q, A, |
| | | or A, X₅ is | X₅ is R, A, | Q, | NO: 263) | or Y, |
| | | A, N, H, S, | Q, or S, | X₅ is Y or | | X₅ is A, T, |
| | | T, F, or Y, | X₆ is A or | S, X₆ is A | | R, S, K, or |
| | | X₆ is A, D, | D, | or V (SEQ | | Y |
| | | or G, | X₇ is D, E, | ID NO: | | (SEQ ID |
| | | X₇ is T, E, | A, or S | 262) | | NO: 264) |
| | | K, V, Q, or | (SEQ ID | | | |
| | | A, | NO: 261) | | | |
| | | X₈ is A or | | | | |
| | | T, X₉ is N | | | | |
| | | or K, X₁₀ is | | | | |
| | | A or N, X₁₁ | | | | |
| | | is Q or T | | | | |
| | | (SEQ ID | | | | |
| | | NO: 260) | | | | |
| V11 family | X₁YX₂MS | DIX₁X₂X₃ | X₁X₂X₃YX | RASQSVS | GASSRAT | QQYGSSP |
| | X₁ is S, D, | GX₄X₅TX₆ | ₄WX₅X₆AX | SNLA | (SEQ ID | PVT |
| | E, A, or Q | YADSVK | ₇DX₈ | (SEQ ID | NO: 41) | (SEQ ID |
| | X₂ is A, G, | G | X₁ is S or | NO: 40) | | NO: 42) |
| | or T (SEQ | X₁ is T, N, | A, X₂ is N, | | | |
| | ID NO: | S, Q, E, H, | H, A, D, L, | | | |
| | 268) | R, or A, | Q, Y, or R, | | | |
| | | X₂ is Y, W, | X₃ is A, N, | | | |
| | | F, or H, | S, or G, | | | |
| | | X₃ is A, S, | X₄ is A, V, | | | |
| | | Q, E, or T, | R, E, or S, | | | |
| | | X₄ is G or | X₅ is D or | | | |
| | | E, X₅ is S | S, X₆ is D, | | | |
| | | or I, | N, Q, E, S, | | | |
| | | X₆ is A or | T, or L, | | | |
| | | G (SEQ ID | X₇ is L, F, | | | |
| | | NO: 269) | or M, | | | |
| | | | X₈ is I, Y, | | | |
| | | | or V | | | |
| | | | (SEQ ID | | | |
| | | | NO: 270) | | | |

TABLE 2
Anti-CD8 antibody CDRs (Chothia)

| Name | CDR-H1 | CDR-H2 | CDR-H3 | CDR-L1 | CDR-L2 | CDR-L3 |
|---|---|---|---|---|---|---|
| xhCD8v1 | GYTFTKY | NPNNDE | DGLGLRL | GASENIY | GATNLAD | QNILDTP |
| | (SEQ ID | (SEQ ID | FAD | GALN | (SEQ ID | WT |
| | NO: 49) | NO: 50) | (SEQ ID | (SEQ ID | NO: 5) | (SEQ ID |
| | | | NO: 3) | NO: 4) | | NO: 6) |
| xhCD8v1.1 | GYTFTKY | NPNNDE | DGLGLRL | RASENIY | GATNLAD | QNILDTP |
| | (SEQ ID | (SEQ ID | FAD | GALN | (SEQ ID | WT |
| | NO: 49) | NO: 50) | (SEQ ID | (SEQ ID | NO: 11) | (SEQ ID |
| | | | NO: 9) | NO: 10) | | NO: 12) |
| xhCD8v2 | GYRFHNF | IPGHAK | DGLGIRL | RASQEIY | GATNLQS | QDIYDAP |
| | (SEQ ID | (SEQ ID | FAD | GALN | (SEQ ID | WT |
| | NO: 51) | NO: 52) | (SEQ ID | (SEQ ID | NO: 17) | (SEQ ID |
| | | | NO: 15) | NO: 16) | | NO: 18) |
| xhCD8v3 | GSRFYKF | IPGHAK | DGLGIRL | RASQEIY | GATNLQS | QDIYDAP |
| | (SEQ ID | (SEQ ID | FAD | GALN | (SEQ ID | WT |
| | NO: 53) | NO: 52) | (SEQ ID | (SEQ ID | NO: 23) | (SEQ ID |
| | | | NO: 21) | NO: 22) | | NO: 24) |
| xhCD8v4 | GYTFTKY | IPGHAK | DGLGIRL | RASQKIY | GATNLQS | QNTYDTP |
| | (SEQ ID | (SEQ ID | FAD | GALN | (SEQ ID | WT |
| | NO: 49) | NO: 52) | (SEQ ID | (SEQ ID | NO: 29) | (SEQ ID |
| | | | NO: 27) | NO: 28) | | NO: 30) |
| xhCD8v5 | GSGFRGH | IPGHAK | DGLGIRL | RASQKIY | GATNLQS | QNTYDTP |
| | (SEQ ID | (SEQ ID | FAD | GALN | (SEQ ID | WT |
| | NO: 54) | NO: 52) | (SEQ ID | (SEQ ID | NO: 35) | (SEQ ID |
| | | | NO: 33) | NO: 34) | | NO: 36) |
| xhCD8v6 | GFTFDDY | NWSGEI | SNSYRW | RASQSVS | GASSRAT | QQYGSSP |
| | (SEQ ID | (SEQ ID | DDALDI | SNLA | (SEQ ID | PVT |
| | NO: 55) | NO: 56) | (SEQ ID | (SEQ ID | NO: 41) | (SEQ ID |
| | | | NO: 39) | NO: 40) | | NO: 42) |
| xhCD8v7 | GFTFDDY | SYDGSN | DRIGWYD | RASHSVG | DASNRAT | QQRSNW |
| | (SEQ ID | (SEQ ID | YDAFDI | SNLA | (SEQ ID | PPT |
| | NO: 55) | NO: 57) | (SEQ ID | (SEQ ID | NO: 47) | (SEQ ID |
| | | | NO: 45) | NO: 46) | | NO: 48) |
| xhCD8v8 | GYAFSSY | YPGDGD | SGAAFSS | RASENIY | AATNLAD | QHFWGTP |
| | (SEQ ID | (SEQ ID | YYAMDY | SNLA | (SEQ ID | WT (SEQ |
| | NO: 183) | NO: 184) | (SEQ ID | (SEQ ID | NO: 181) | ID NO: |
| | | | NO: 179) | NO: 180) | | 182) |
| xhCD8v9 | GGTFSSY | IPGAAT | DAAGIRL | RASQEIY | GATNLQS | QSTYDAP |
| | (SEQ ID | (SEQ ID | FAD (SEQ | GALN | (SEQ ID | WT (SEQ |
| | NO: 238) | NO: 239) | ID NO: | (SEQ ID | NO: 17) | ID NO: |
| | | | 233) | NO: 16) | | 228) |
| xhCD8v10 | GFTFSSY | TYAGGS | SNAYAW | RASQSVS | GASSRAT | QQYGSSP |
| | (SEQ ID | (SEQ ID | DDALDI | SNLA | (SEQ ID | PVT |
| | NO: 240) | NO: 241) | (SEQ ID | (SEQ ID | NO: 41) | (SEQ ID |
| | | | NO: 242) | NO: 40) | | NO: 42) |
| xhCD8v11 | GFTFSSY | TYAGGS | SNAYAW | RASQSVS | GASSRAT | QQYGSSP |
| | (SEQ ID | (SEQ ID | DDALDI | SNLA | (SEQ ID | PVT |
| | NO: 240) | NO: 241) | (SEQ ID | (SEQ ID | NO: 41) | (SEQ ID |
| | | | NO: 242) | NO: 40) | | NO: 42) |
| xhCD8v12 | GGTFSSY | IPGYAT | DAAGIRL | RASQSIY | GASNLQS | QSTYTAP |
| | (SEQ ID | (SEQ ID | FAD (SEQ | GALN | (SEQ ID | WT (SEQ |
| | NO: 238) | NO: 243) | ID NO: | (SEQ ID | NO: 235) | ID NO: |
| | | | 233) | NO: 234) | | 236) |
| xhCD8v13 | GGTFSSY | IPGYAT | DAAGIRL | RASQEIY | GATNLQS | QSTYDAP |
| | (SEQ ID | (SEQ ID | FAD (SEQ | GALN | (SEQ ID | WT (SEQ |
| | NO: 238) | NO: 243) | ID NO: | (SEQ ID | NO: 17) | ID NO: |
| | | | 233) | NO: 16) | | 228) |
| xhCD8v14 | GFTFSSY | SYAGGS | SNAYAW | RASQSVS | GASSRAT | QQYGSSP |
| | (SEQ ID | (SEQ ID | DDALDI | SNLA | (SEQ ID | PVT |
| | NO: 240) | NO: 244) | (SEQ ID | (SEQ ID | NO: 41) | (SEQ ID |
| | | | NO: 242) | NO: 40) | | NO: 42) |
| xhCD8v15 | GFTFSSY | SYAGGS | SNAYAW | RASQSVS | GASSRAT | QQYGSSP |
| | (SEQ ID | (SEQ ID | DDALDI | SNLA | (SEQ ID | PVT |
| | NO: 240) | NO: 244) | (SEQ ID | (SEQ ID | NO: 41) | (SEQ ID |
| | | | NO: 242) | NO: 40) | | NO: 42) |
| V9 family | GX₁X₂FX₃ | X₁PX₂X₃X₄ | X₁X₂X₃GX | X₁X₂SX₃X₄ | GX₁X₂X₃L | QX₁X₂X₃X |
| | X₄X₅ | X₅ | ₄X₅LFX₆X₇ | IX₅GX₆LN | X₄X₅ | ₄X₅PWT |
| | X₁ is G, Y, | X₁ is I, N, | X₁ is D or | X₁ is R or | X₁ is A or | X₁ is S, N, |
| | S, or A, | or M, | A, | G, | S, X₂ is T, | D, Q, A, or |
| | X₂ is T, S, | X₂ is G, N, | X₂ is A, G, | X₂ is A or | S, E, Q, or | E, |
| | G, R, N, or | H, S, R, I, | E, R, Y, K, | T, | D, X₃ is N, | X₂ is T, I, |
| | H, | or A, | N, Q, L, or | X₃ is Q or | R, A, E, or | or S, |
| | X₃ is S, T, | X₃ is A, N, | F, | E, | H, X₄ is Q | X₃ is Y, L, |
| | R, H, Y, G, | H, S, T, F, | X₃ is A, L, | X₄ is E, N, | or A, | or F, |
| | or P, | or Y, | P, or Y, | T, S, A, K, | X₅ is S or D | X₄ is D, G, |
| | X₄ is S, K, | X₄ is A, D, | X₄ is I or L, | D, G, R, or | (SEQ ID | T, E, Q, A, |
| | G, N, R, D, | or G, | X₅ is R, A, | Q, | NO: 263) | or Y, |
| | T, or G, | X₅ is T, E, | Q, or S, | X₅ is Y or | | X₅ is A, T, |
| | X₅ is Y, L, | K, V, Q, or | X₆ is A or | S, X₆ is A | | R, S, K, or |
| | H, or F | A | D, | or V (SEQ | | Y |
| | (SEQ ID | (SEQ ID | X₇ is D, E, | ID NO: | | (SEQ ID |
| | NO: 265) | NO: 266) | A, or S | 262) | | NO: 264) |
| | | | (SEQ ID | | | |
| | | | NO: 267) | | | |
| V11 family | GFTFX₁X₂ | X₁X₂X₃GX | X₁X₂X₃YX | RASQSVS | GASSRAT | QQYGSSP |
| | Y | ₄X₅ | ₄WX₅X₆AX | SNLA | (SEQ ID | PVT |
| | X₁ is S, D, | X₁ is T, N, | ZDX₈ | (SEQ ID | NO: 41) | (SEQ ID |
| | E, Q, S, or | S, Q, E, H, | X₁ is S or | NO: 40) | | NO: 42) |
| | A | R or A, | A, X₂ is N, | | | |
| | X₂ is S, D, | X₂ is Y, W, | H, A, D, L, | | | |
| | E, A, or Q | F, or H, | Q, Y, or R, | | | |
| | (SEQ ID | X₃ is A, S, | X₃ is A, N, | | | |
| | NO: 271) | Q, E, or T, | S, or G, | | | |
| | | X₄ is G or | X₄ is A, V, | | | |
| | | E, | R, E, or S, | | | |
| | | X₅ is S or I | X₅ is D or | | | |
| | | (SEQ ID | S, X₆ is D, | | | |
| | | NO: 272) | N, Q, E, S, | | | |
| | | | T, or L, | | | |
| | | | X₇ is L, F, | | | |
| | | | or M, | | | |
| | | | X₈ is I, Y, | | | |
| | | | or V | | | |
| | | | (SEQ ID | | | |
| | | | NO: 273) | | | |

TABLE 3
Anti-CD8 antibody variable domain sequences

| Name | VH | VL |
|---|---|---|
| xhCD8v1 | QVHLQQSGPELVKPGASVKMSCKT | DIQMTQSPASLSASVGETVTITCGA |
| | SGYTFTKYTMHWVKQGHEESLEWI | SENIYGALNWYQRKQGKSPQLLIFG |
| | GHFNPNNDETKYNQKFTGKATLTV | ATNLADGVSSRFSGSGSDRQYSLKI |
| | DKSSTTAYMELRSLTSDDSALYYC | SSLHPDDVATYYCQNILDTPWTFG |
| | ARDGLGLRLFADWGQGTLITVSA | GGTKLEIK |
| | (SEQ ID NO: 58) | (SEQ ID NO: 59) |
| xhCD8v1.1 | QVQLVQSGAEVKKPGSSVKVSCKA | DIQMTQSPSSLSASVGDRVTITCRA |
| | SGYTFTKYAISWVRQAPGQGLEW | SENIYGALNWYQQKPGKAPKLLIY |
| | MGHFNPNNDETKYNQKFQGRVTIT | GATNLADGVPSRFSGSGSGTDFTLT |
| | ADESTSTAYMELSSLRSEDTAVYY | ISSLQPEDFATYYCQNILDTPWTFG |
| | CARDGLGLRLFADWGQGT | GGTKLEIK |
| | LVTVSS | (SEQ ID NO: 61) |
| | (SEQ ID NO: 60) | |
| xhCD8v2 | QVQLVQSGAEVKKPGSSVKVSCKA | DIQMTQSPSSLSASVGDRVTITCRA |
| | SGYRFHNFAISWVRQAPGQGLEW | SQEIYGALNWYQQKPGKAPKLLIY |
| | MGGIIPGHAKANYAQKFQGRVTIT | GATNLQSGVPSRFSGSGSGTDFTLTI |
| | ADESTSTAYMELSSLRSEDTAVYY | SSLQPEDFATYYCQDIYDAPWTFG |
| | CARDGLGIRLFADWGQGTLVTVSS | GGTKVEIK |
| | (SEQ ID NO: 62) | (SEQ ID NO: 63) |
| xhCD8v3 | QVQLVQSGAEVKKPGSSVKVSCKA | DIQMTQSPSSLSASVGDRVTITCRA |
| | SGSRFYKFAISWVRQAPGQGLEWM | SQEIYGALNWYQQKPGKAPKLLIY |
| | GGIIPGHAKANYAQKFQGRVTITAD | GATNLQSGVPSRFSGSGSGTDFTLTI |
| | ESTSTAYMELSSLRSEDTAVYYCAR | SSLQPEDFATYYCQDIYDAPWTFG |
| | DGLGIRLFADWGQGTLVTVSS | GGTKVEIK |
| | (SEQ ID NO: 64) | (SEQ ID NO: 65) |
| xhCD8v4 | QVQLVQSGAEVKKPGSSVKVSCKA | DIQMTQSPSSLSASVGDRVTITCRA |
| | SGYTFTKYAISWVRQAPGQGLEW | SQKIYGALNWYQQKPGKAPKLLIY |
| | MGGIIPGHAKANYAQKFQGRVTIT | GATNLQSGVPSRFSGSGSGTDFTLTI |
| | ADESTSTAYMELSSLRSEDTAVYY | SSLQPEDFATYYCQNTYDTPWTFG |
| | CARDGLGIRLFADWGQGTLVTVSS | GGTKVEIK |
| | (SEQ ID NO: 66) | (SEQ ID NO: 67) |
| xhCD8v5 | QVQLVQSGAEVKKPGSSVKVSCKA | DIQMTQSPSSLSASVGDRVTITCRA |
| | SGSGFRGHAISWVRQAPGQGLEW | SQKIYGALNWYQQKPGKAPKLLIY |
| | MGGIIPGHAKANYAQKFQGRVTIT | GATNLQSGVPSRFSGSGSGTDFTLTI |
| | ADESTSTAYMELSSLRSEDTAVYY | SSLQPEDFATYYCQNTYDTPWTFG |
| | CARDGLGIRLFADWGQGTLVTVSS | GGTKVEIK |
| | (SEQ ID NO: 68) | (SEQ ID NO: 69) |
| xhCD8v6 | EQLVESGGGAVRPGGSLRLSCAA | EIVLTQSPATLSVSPGERATLSCRAS |
| | SGFTFDDYGMSWVRQAPGKGLEW | QSVSSNLAWYQQKPGQAPRLLIYG |
| | VSDINWSGEITAYADSVKGRFTISR | ASSRATGIPDRFSGSGSGTDFTLTIS |
| | DNAKNSLYLQMNSLRAEDTAVYY | RLEPEDFAVYYCQQYGSSPPVTFGQ |
| | CARSNSYRWDDALDIWGQGTMVT | GTKVEIK |
| | VSS | (SEQ ID NO: 71) |
| | (SEQ ID NO: 70) | |
| xhCD8v7 | EVQLVESGGGLVQPGRSLRLSCAA | EIVLTQSPATLSVTPGEGATLSCRAS |
| | SGFTFDDYAMHWVRQAPGKGLEW | HSVGSNLAWYQQKPGQAPRLLIYD |
| | VAVISYDGSNKYYADSVKGRFTISR | ASNRATGIPARFSGSGSGTDFTLTIS |
| | DNSKNTLYLQMNSLRAEDTAVYY | SLEPEDLAVYYCQQRSNWPPTFGQ |
| | CAKDRIGWYDYDAFDIWGQGTMV | GTRLEIK |
| | TVSS | (SEQ ID NO: 73) |
| | (SEQ ID NO: 72) | |
| xhCD8v8 | QVQLQQSGAELVRPGSSVKISCKAS | DIQMTQSPASLSVSVGETVTITCRA |
| | GYAFSSYWMNWVKQRPGQGLEWI | SENIYSNLAWYQQKQGKSPQLLVY |
| | GQIYPGDGDTNYNGKFKGKATLTA | AATNLADGVPSRFSGSGSGTQYSL |
| | DKSSSTAYMQLSSLTSEDSAVYFCA | KINSLQSEDFGSYYCQHFWGTPWT |
| | RSGAAFSSYYAMDYWGQGTSVTV | FGGGTKLEIK (SEQ ID NO: 186) |
| | SS (SEQ ID NO: 185) | |
| xhCD8v9 | QVQLVQSGAEVKKPGSSVKVSCKA | DIQMTQSPSSLSASVGDRVTITCRA |
| | SGGTFSSYAISWVRQAPGQGLEWM | SQEIYGALNWYQQKPGKAPKLLIY |
| | GGIIPGAATANYAQKFQGRVTITAD | GATNLQSGVPSRFSGSGSGTDFTLTI |
| | ESTSTAYMELSSLRSEDTAVYYCAR | SSLQPEDFATYYCQSTYDAPWTFG |
| | DAAGIRLFADWGQGTLVTVSS | GGTKVEIK (SEQ ID NO: 246) |
| | (SEQ ID NO: 245) | |
| xhCD8v10 | EVQLVESGGGLVQPGGSLRLSCAA | EIVLTQSPGTLSLSPGERATLSCRAS |
| | SGFTFSSYAMSWVRQAPGKGLEW | QSVSSNLAWYQQKPGQAPRLLIYG |
| | VSDITYAGGSTAYADSVKGRFTISR | ASSRATGIPDRFSGSGSGTDFTLTIS |
| | DNAKNSLYLQMNSLRAEDTAVYY | RLEPEDFAVYYCQQYGSSPPVTFGQ |
| | CARSNAYAWDDALDIWGQGTMVT | GTKVEIK (SEQ ID NO: 248) |
| | VSS (SEQ ID NO: 247) | |
| xhCD8v11 | EVQLVESGGGLVQPGGSLRLSCAA | EIVLTQSPGTLSLSPGERATLSCRAS |
| | SGFTFSSYAMSWVRQAPGKGLEW | QSVSSNLAWYQQKPGQAPRLLIYG |
| | VSDITYAGGSTAYADSVKGRFTISR | ASSRATGIPDRFSGSGSGTDFTLTIS |
| | DNAKNSLYLQMNSLRAEDTAVYY | RLEPEDFAVYYCQQYGSSPPVTFGQ |
| | CARSNAYAWDDALDIWGQGTLVT | GTKVEIK (SEQ ID NO: 250) |
| | VSS (SEQ ID NO: 249) | |
| xhCD8v12 | QVQLVQSGAEVKKPGSSVKVSCKA | DIQMTQSPSSLSASVGDRVTITCRA |
| | SGGTFSSYAISWVRQAPGQGLEWM | SQSIYGALNWYQQKPGKAPKLLIY |
| | GGIIPGYATANYAQKFQGRVTITAD | GASNLQSGVPSRFSGSGSGTDFTLTI |
| | ESTSTAYMELSSLRSEDTAVYYCAR | SSLQPEDFATYYCQSTYTAPWTFG |
| | DAAGIRLFADWGQGTLVTVSS | GGTKVEIK (SEQ ID NO: 252) |
| | (SEQ ID NO: 251) | |
| xhCD8v13 | QVQLVQSGAEVKKPGSSVKVSCKA | DIQMTQSPSSLSASVGDRVTITCRA |
| | SGGTFSSYAISWVRQAPGQGLEWM | SQEIYGALNWYQQKPGKAPKLLIY |
| | GGIIPGYATANYAQKFQGRVTITAD | GATNLQSGVPSRFSGSGSGTDFTLTI |
| | ESTSTAYMELSSLRSEDTAVYYCAR | SSLQPEDFATYYCQSTYDAPWTFG |
| | DAAGIRLFADWGQGTLVTVSS | GGTKVEIK (SEQ ID NO: 254) |
| | (SEQ ID NO: 253) | |
| xhCD8v14 | EVQLVESGGGLVQPGGSLRLSCAA | EIVLTQSPGTLSLSPGERATLSCRAS |
| | SGFTFSSYAMSWVRQAPGKGLEW | QSVSSNLAWYQQKPGQAPRLLIYG |
| | VSDISYAGGSTAYADSVKGRFTISR | ASSRATGIPDRFSGSGSGTDFTLTIS |
| | DNAKNSLYLQMNSLRAEDTAVYY | RLEPEDFAVYYCQQYGSSPPVTFGQ |
| | CARSNAYAWDDALDIWGQGTMVT | GTKVEIK (SEQ ID NO: 256) |
| | VSS (SEQ ID NO: 255) | |
| xhCD8v15 | EVQLVESGGGLVQPGGSLRLSCAA | EIVLTQSPGTLSLSPGERATLSCRAS |
| | SGFTFSSYAMSWVRQAPGKGLEW | QSVSSNLAWYQQKPGQAPRLLIYG |
| | VSDISYAGGSTAYADSVKGRFTISR | ASSRATGIPDRFSGSGSGTDFTLTIS |
| | DNAKNSLYLQMNSLRAEDTAVYY | RLEPEDFAVYYCQQYGSSPPVTFGQ |
| | CARSNAYAWDDALDIWGQGTLVT | GTKVEIK (SEQ ID NO: 258) |
| | VSS (SEQ ID NO: 257) | |

### Fusion Protein Cytokines

In some embodiments, the second moiety induces activation of CD8+ T cells. In some embodiments, the second moiety comprises a polypeptide that induces signaling via IL-2Rβγ. In some embodiments, the second moiety comprises a polypeptide that induces signaling via IL-21R. In some embodiments, the second moiety comprises an IL-21 polypeptide (e.g., a human IL-21 polypeptide or derivative thereof). In some embodiments, the second moiety comprises an IL-2 polypeptide (e.g., a human IL-2 polypeptide or derivative thereof).

### IL-2 Polypeptides

In some embodiments, the second moiety comprises an IL-2 polypeptide (e.g., a human IL-2 polypeptide or derivative thereof).

In some embodiments, the IL-2 polypeptide has a binding affinity to IL-2Ru that is reduced by 50% or more, compared to binding affinity of a wild-type IL-2 polypeptide comprising the sequence of SEQ ID NO: 81 for IL-2Ru. In some embodiments, “wild-type IL-2” and “human IL-2” are used interchangeably and refer to a polypeptide having the amino acid sequence of SEQ ID NO: 81. In some embodiments, the IL-2 polypeptide has a binding affinity to IL-2RD that is reduced by 50% or more, compared to binding affinity of a wild-type IL-2 polypeptide comprising the sequence of SEQ ID NO: 81 for IL-2RD. In some embodiments, the IL-2 polypeptide has a binding affinity to IL-2Rγ that is reduced by 50% or more, compared to binding affinity of a wild-type IL-2 polypeptide comprising the sequence of SEQ ID NO: 81 for IL-2Rγ. Differences in binding affinity of wild-type and disclosed polypeptide for IL-2Rα and IL-2Rβ can be measured, e.g., in standard surface plasmon resonance (SPR) assays that measure affinity of protein-protein interactions familiar to those skilled in the art. Differences in binding affinity of wild-type and disclosed polypeptide for IL-2Rγ cannot reliably be measured by SPR assays as the affinity of wild-type IL-2 polypeptide for IL-2Rγ is very low. Instead, their reduced affinity to IL-2Rγ can be deduced by performing an in vitro assay that measures pSTAT5 and compares the activity of IL-2 polypeptides with and without the IL-2Rγ affinity-reducing substitution on IL-2R-expressing cells. Exemplary sequences for IL-2Rα, IL-2RD, and IL-2Rγ are provided below.

IL-2Rα:
(SEQ ID NO: 82)
MDSYLLMWGLLTFIMVPGCQAELCDDDPPEIPHATFKAMAYKEGTMLNC
ECKRGFRRIKSGSLYMLCTGNSSHSSWDNQCQCTSSATRNTTKQVTPQP
EEQKERKTTEMQSPMQPVDQASLPGHCREPPPWENEATERIYHFVVGQM
VYYQCVQGYRALHRGPAESVCKMTHGKTRWTQPQLICTGEMETSQFPGE
EKPQASPEGRPESETSCLVTTTDFQIQTEMAATMETSIFTTEYQVAVAG
CVFLLISVLLLSGLTWQRRQRKSRRTI
IL-2Rβ:
(SEQ ID NO: 83)
MAAPALSWRLPLLILLLPLATSWASAAVNGTSQFTCFYNSRANISCVWS
QDGALQDTSCQVHAWPDRRRWNQTCELLPVSQASWACNLILGAPDSQKL
TTVDIVTLRVLCREGVRWRVMAIQDFKPFENLRLMAPISLQVVHVETHR
CNISWEISQASHYFERHLEFEARTLSPGHTWEEAPLLTLKQKQEWICLE
TLTPDTQYEFQVRVKPLQGEFTTWSPWSQPLAFRTKPAALGKDTIPWLG
HLLVGLSGAFGFIILVYLLINCRNTGPWLKKVLKCNTPDPSKFFSQLSS
EHGGDVQKWLSSPFPSSSFSPGGLAPEISPLEVLERDKVTQLLLQQDKV
PEPASLSSNHSLTSCFTNQGYFFFHLPDALEIEACQVYFTYDPYSEEDP
DEGVAGAPTGSSPQPLQPLSGEDDAYCTFPSRDDLLLFSPSLLGGPSPP
STAPGGSGAGEERMPPSLQERVPRDWDPQPLGPPTPGVPDLVDFQPPPE
LVLREAGEEVPDAGPREGVSFPWSRPPGQGEFRALNARLPLNTDAYLSL
QELQGQDPTHLV
IL-2Rγ:
(SEQ ID NO: 84)
MLKPSLPFTSLLFLQLPLLGVGLNTTILTPNGNEDTTADFFLTTMPTDS
LSVSTLPLPEVQCFVFNVEYMNCTWNSSSEPQPTNLTLHYWYKNSDNDK
VQKCSHYLFSEEITSGCQLQKKEIHLYQTFVVQLQDPREPRRQATQMLK
LQNLVIPWAPENLTLHKLSESQLELNWNNRFLNHCLEHLVQYRTDWDHS
WTEQSVDYRHKFSLPSVDGQKRYTFRVRSRFNPLCGSAQHWSEWSHPIH
WGSNTSKENPFLFALEAVVISVGSMGLIISLLCVYFWLERTMPRIPTLK
NLEDLVTEYHGNFSAWSGVSKGLAESLQPDYSERLCLVSEIPPKGGALG
EGPGASPCNQHSPYWAPPCYTLKPET

In some embodiments, the IL-2 polypeptide is a IL-2 polypeptide comprising one or more mutations relative to a human IL-2 polypeptide comprising the sequence of APTSSSTKKTQLQLEHLLLDLQMILNGINNYKNPKLTRMLTFKFYMPKKATELKHLQ CLEEELKPLEEVLNLAQSKNFHLRPRDLISNINVIVLELKGSETTFMCEYADETATIVE FLNRWITFCQSIISTLT (SEQ ID NO: 81). In some embodiments, the IL-2 polypeptides of the present disclosure have one or more, two or more, or three or more affinity-reducing amino acid substitutions relative to the wild-type mature IL-2 polypeptide, e.g., having the amino acid sequence of SEQ ID NO: 81. In some embodiments, one or more, two or more, or three or more substituted residues, are selected from the following group: Q11, H16, L18, L19, D20, D84, S87, Q22, R38, F42, K43, Y45, E62, P65, E68, V69, L72, D84, S87, N88, V91, 192, T123, Q126, S127, 1129, and S130. Decreased affinity to IL-2Rα may be obtained by substituting one or more of the following residues in the sequence of the wild-type mature IL-2 polypeptide: R38, F42, K43, Y45, E62, P65, E68, V69, and L72. Decreased affinity to IL-2Rβ may be obtained by substituting one or more of the following residues: E15, H16, L19, D20, D84, S87, N88, V91, and 192. Decreased affinity to IL-2Rγ may be obtained by substituting one or more of the following residues in the sequence of the wild-type mature IL-2 polypeptide: Q11, L18, Q22, T123, Q126, S127, 1129, and S130.

In some embodiments, the IL-2 polypeptide comprises an F42A or F42K amino acid substitution relative to the wild-type mature IL-2 amino acid sequence. In some embodiments, the IL-2 polypeptide comprises an F42A or F42K amino acid substitution and an R38A, R38D, R38E, E62Q, E68A, E68Q, E68K, or E68R amino acid substitution relative to the wild-type mature IL-2 amino acid sequence. For example, in some embodiments, the IL-2 polypeptide comprises F42A; R38A and F42A; R38D and F42A; R38E and F42A; F42A and E62Q; F42A and E68A; F42A and E68Q; F42A and E68K; F42A and E68R; or R38A and F42K amino acid substitution(s) relative to the wild-type mature IL-2 amino acid sequence, e.g., as shown in SEQ ID NO: 81. In some embodiments, the IL-2 polypeptide comprises R38E and F42A amino acid substitutions relative to the wild-type IL-2 amino acid sequence. In some embodiments, the IL-2 polypeptide comprises R38D and F42A amino acid substitutions relative to the wild-type IL-2 amino acid sequence. In some embodiments, the IL-2 polypeptide comprises F42A and E62Q amino acid substitutions relative to the wild-type IL-2 amino acid sequence. In some embodiments, the IL-2 polypeptide comprises R38A and F42K amino acid substitutions relative to the wild-type IL-2 amino acid sequence. In some embodiments, the IL-2 polypeptide comprises R38D and F42A amino acid substitutions relative to the wild-type IL-2 amino acid sequence. In some embodiments, the IL-2 polypeptide comprises R38A and F42K amino acid substitutions relative to the wild-type IL-2 amino acid sequence. In some embodiments, the IL-2 polypeptide comprises F42A and E62Q amino acid substitutions relative to the wild-type IL-2 amino acid sequence. In some embodiments, the IL-2 polypeptide comprises an H16E, H16D, D20N, M23A, M23R, M23K, D84L, D84N, D84V, D84H, D84Y, D84R, D84K, S87K, S87A, N88A, N88D, N88G, N88S, N88T, N88R, N88I, V91A, V91T, V91E, I92A, E95S, E95A, E95R, T123A, T123E, T123K, T123Q, Q126A, Q126S, Q126T, Q126E, S127A, S127E, S127K, or S127Q amino acid substitution relative to the wild-type IL-2 amino acid sequence. In some embodiments, the IL-2 polypeptide comprises F42A; R38A and F42A; R38D and F42A; R38E and F42A; F42A and E62Q; F42A and E68A; F42A and E68Q; F42A and E68K; F42A and E68R; or R38A and F42K amino acid substitution(s) relative to the wild-type mature IL-2 amino acid sequence and an H16E, H16D, D20N, M23A, M23R, M23K, D84L, D84N, D84V, D84H, D84Y, D84R, D84K, S87K, S87A, N88A, N88D, N88G, N88S, N88T, N88R, N88I, V91A, V91T, V91E, 192A, E95S, E95A, E95R, T123A, T123E, T123K, T123Q, Q126A, Q126S, Q126T, Q126E, S127A, S127E, S127K, or S127Q amino acid substitution relative to the wild-type mature IL-2 amino acid sequence, e.g., as shown in SEQ ID NO: 81. For example, in some embodiments, the IL-2 polypeptide comprises R38E, F42A, and H16E amino acid substitutions relative to the wild-type IL-2 amino acid sequence. In some embodiments, the IL-2 polypeptide comprises R38E, F42A, and H16D amino acid substitutions relative to the wild-type IL-2 amino acid sequence. In some embodiments, the IL-2 polypeptide comprises R38E, F42A, and D84K amino acid substitutions relative to the wild-type IL-2 amino acid sequence. In some embodiments, the IL-2 polypeptide comprises R38E, F42A, and D84R amino acid substitutions relative to the wild-type IL-2 amino acid sequence. In some embodiments, the IL-2 polypeptide comprises R38E, F42A, and N88S amino acid substitutions relative to the wild-type IL-2 amino acid sequence. In some embodiments, the IL-2 polypeptide comprises R38E, F42A, and N88A amino acid substitutions relative to the wild-type IL-2 amino acid sequence. In some embodiments, the IL-2 polypeptide comprises R38E, F42A, and N88G amino acid substitutions relative to the wild-type IL-2 amino acid sequence. In some embodiments, the IL-2 polypeptide comprises R38E, F42A, and N88R amino acid substitutions relative to the wild-type IL-2 amino acid sequence. In some embodiments, the IL-2 polypeptide comprises R38E, F42A, and N88T amino acid substitutions relative to the wild-type IL-2 amino acid sequence. In some embodiments, the IL-2 polypeptide comprises R38E, F42A, and N88D amino acid substitutions relative to the wild-type IL-2 amino acid sequence. In some embodiments, the IL-2 polypeptide comprises R38E, F42A, and V91E amino acid substitutions relative to the wild-type IL-2 amino acid sequence. In some embodiments, the IL-2 polypeptide comprises R38E, F42A, and Q126S amino acid substitutions relative to the wild-type IL-2 amino acid sequence. In some embodiments, the IL-2 polypeptide comprises the amino acid sequence of SEQ ID NO: 81 with one of the following sets of amino acid substitutions (relative to the sequence of SEQ ID NO: 81): R38E and F42A; R38D and F42A; F42A and E62Q; R38A and F42K; R38E, F42A, and N88S; R38E, F42A, and N88A; R38E, F42A, and N88G; R38E, F42A, and N88R; R38E, F42A, and N88T; R38E, F42A, and N88D; R38E, F42A, and V91E; R38E, F42A, and D84H; R38E, F42A, and D84K; R38E, F42A, and D84R; H16D, R38E and F42A; H16E, R38E and F42A; R38E, F42A and Q126S; R38D, F42A and N88S; R38D, F42A and N88A; R38D, F42A and N88G; R38D, F42A and N88R; R38D, F42A and N88T; R38D, F42A and N88D; R38D, F42A and V91E; R38D, F42A, and D84H; R38D, F42A, and D84K; R38D, F42A, and D84R; H16D, R38D and F42A; H16E, R38D and F42A; R38D, F42A and Q126S; R38A, F42K, and N88S; R38A, F42K, and N88A; R38A, F42K, and N88G; R38A, F42K, and N88R; R38A, F42K, and N88T; R38A, F42K, and N88D; R38A, F42K, and V91E; R38A, F42K, and D84H; R38A, F42K, and D84K; R38A, F42K, and D84R; H16D, R38A, and F42K; H16E, R38A, and F42K; R38A, F42K, and Q126S; F42A, E62Q, and N88S; F42A, E62Q, and N88A; F42A, E62Q, and N88G; F42A, E62Q, and N88R; F42A, E62Q, and N88T; F42A, E62Q, and N88D; F42A, E62Q, and V91E; F42A, E62Q, and D84H; F42A, E62Q, and D84K; F42A, E62Q, and D84R; H16D, F42A, and E62Q; H16E, F42A, and E62Q; F42A, E62Q, and Q126S; R38E, F42A, and C125A; R38D, F42A, and C125A; F42A, E62Q, and C125A; R38A, F42K, and C125A; R38E, F42A, N88S, and C125A; R38E, F42A, N88A, and C125A; R38E, F42A, N88G, and C125A; R38E, F42A, N88R, and C125A; R38E, F42A, N88T, and C125A; R38E, F42A, N88D, and C125A; R38E, F42A, V91E, and C125A; R38E, F42A, D84H, and C125A; R38E, F42A, D84K, and C125A; R38E, F42A, D84R, and C125A; H16D, R38E, F42A, and C125A; H16E, R38E, F42A, and C125A; R38E, F42A, C125A and Q126S; R38D, F42A, N88S, and C125A; R38D, F42A, N88A, and C125A; R38D, F42A, N88G, and C125A; R38D, F42A, N88R, and C125A; R38D, F42A, N88T, and C125A; R38D, F42A, N88D, and C125A; R38D, F42A, V91E, and C125A; R38D, F42A, D84H, and C125A; R38D, F42A, D84K, and C125A; R38D, F42A, D84R, and C125A; H16D, R38D, F42A, and C125A; H16E, R38D, F42A, and C125A; R38D, F42A, C125A, and Q126S; R38A, F42K, N88S, and C125A; R38A, F42K, N88A, and C125A; R38A, F42K, N88G, and C125A; R38A, F42K, N88R, and C125A; R38A, F42K, N88T, and C125A; R38A, F42K, N88D, and C125A; R38A, F42K, V91E, and C125A; R38A, F42K, D84H, and C125A; R38A, F42K, D84K, and C125A; R38A, F42K, D84R, and C125A; H16D, R38A, F42K, and C125A; H16E, R38A, F42K, and C125A; R38A, F42K, C125A and Q126S; F42A, E62Q, N88S, and C125A; F42A, E62Q, N88A, and C125A; F42A, E62Q, N88G, and C125A; F42A, E62Q, N88R, and C125A; F42A, E62Q, N88T, and C125A; F42A, E62Q, N88D, and C125A; F42A, E62Q, V91E, and C125A; F42A, E62Q, and D84H, and C125A; F42A, E62Q, and D84K, and C125A; F42A, E62Q, and D84R, and C125A; H16D, F42A, and E62Q, and C125A; H16E, F42A, E62Q, and C125A; F42A, E62Q, C125A and Q126S; F42A, N88S, and C125A; F42A, N88A, and C125A; F42A, N88G, and C125A; F42A, N88R, and C125A; F42A, N88T, and C125A; F42A, N88D, and C125A; F42A, V91E, and C125A; F42A, D84H, and C125A; F42A, D84K, and C125A; F42A, D84R, and C125A; H16D, F42A, and C125A; H16E, F42A, and C125A; and F42A, C125A and Q126S. In some embodiments, the IL-2 polypeptide comprises the sequence of SEQ ID NO: 81 with one, two, three, four, or five amino acid substitutions relative to SEQ ID NO: 81, and wherein the one, two, three, four, or five substitution(s) comprise substitution(s) at positions of SEQ ID NO: 81 selected from the group consisting of: Q11, H16, L18, L19, D20, Q22, R38, F42, K43, Y45, E62, P65, E68, V69, L72, D84, S87, N88, V91, 192, T123, Q126, S127, 1129, and S130. In some embodiments, the IL-2 polypeptide comprises the sequence of SEQ ID NO: 81 with one of the following sets of amino acid substitutions (relative to the sequence of SEQ ID NO: 81): R38E and F42A; R38D and F42A; F42A and E62Q; R38A and F42K; R38E, F42A, and N88S; R38E, F42A, and N88A; R38E, F42A, and N88G; R38E, F42A, and N88R; R38E, F42A, and N88T; R38E, F42A, and N88D; R38E, F42A, and V91E; R38E, F42A, and D84H; R38E, F42A, and D84K; R38E, F42A, and D84R; H16D, R38E and F42A; H16E, R38E and F42A; R38E, F42A and Q126S; R38D, F42A and N88S; R38D, F42A and N88A; R38D, F42A and N88G; R38D, F42A and N88R; R38D, F42A and N88T; R38D, F42A and N88D; R38D, F42A and V91E; R38D, F42A, and D84H; R38D, F42A, and D84K; R38D, F42A, and D84R; H16D, R38D and F42A; H16E, R38D and F42A; R38D, F42A and Q126S; R38A, F42K, and N88S; R38A, F42K, and N88A; R38A, F42K, and N88G; R38A, F42K, and N88R; R38A, F42K, and N88T; R38A, F42K, and N88D; R38A, F42K, and V91E; R38A, F42K, and D84H; R38A, F42K, and D84K; R38A, F42K, and D84R; H16D, R38A, and F42K; H16E, R38A, and F42K; R38A, F42K, and Q126S; F42A, E62Q, and N88S; F42A, E62Q, and N88A; F42A, E62Q, and N88G; F42A, E62Q, and N88R; F42A, E62Q, and N88T; F42A, E62Q, and N88D; F42A, E62Q, and V91E; F42A, E62Q, and D84H; F42A, E62Q, and D84K; and F42A, E62Q, and D84R. In some embodiments, the IL-2 polypeptide comprises the sequence of SEQ ID NO: 81 with the following amino acid substitutions relative to the sequence of SEQ ID NO: 81: R38D, F42A and N88S. In some embodiments, the IL-2 polypeptide comprises the sequence of SEQ ID NO: 81 with the following amino acid substitutions relative to the sequence of SEQ ID NO: 81: R38D, F42A and N88A. In some embodiments, the IL-2 polypeptide comprises the sequence of SEQ ID NO: 81 with the following amino acid substitutions relative to the sequence of SEQ ID NO: 81: R38D, F42A and N88G. In some embodiments, the IL-2 polypeptide comprises the sequence of SEQ ID NO: 81 with the following amino acid substitutions relative to the sequence of SEQ ID NO: 81: R38D, F42A and N88R. In some embodiments, the IL-2 polypeptide comprises the sequence of SEQ ID NO: 81 with the following amino acid substitutions relative to the sequence of SEQ ID NO: 81: R38D, F42A and N88T. In some embodiments, the IL-2 polypeptide comprises the sequence of SEQ ID NO: 81 with the following amino acid substitutions relative to the sequence of SEQ ID NO: 81: R38D, F42A and N88D. In some embodiments, the IL-2 polypeptide comprises the sequence of SEQ ID NO: 81 with the following amino acid substitutions relative to the sequence of SEQ ID NO: 81: R38D, F42A and V91E. In some embodiments, the IL-2 polypeptide comprises the sequence of SEQ ID NO: 81 with the following amino acid substitutions relative to the sequence of SEQ ID NO: 81: R38D, F42A, and D84H. In some embodiments, the IL-2 polypeptide comprises the sequence of SEQ ID NO: 81 with the following amino acid substitutions relative to the sequence of SEQ ID NO: 81: R38D, F42A, and D84K. In some embodiments, the IL-2 polypeptide comprises the sequence of SEQ ID NO: 81 with the following amino acid substitutions relative to the sequence of SEQ ID NO: 81: R38D, F42A, and D84R. In some embodiments, the IL-2 polypeptide comprises the sequence of SEQ ID NO: 81 with the following amino acid substitutions relative to the sequence of SEQ ID NO: 81: H16D, R38D and F42A. In some embodiments, the IL-2 polypeptide comprises the sequence of SEQ ID NO: 81 with the following amino acid substitutions relative to the sequence of SEQ ID NO: 81: H16E, R38D and F42A. In some embodiments, the IL-2 polypeptide comprises the sequence of SEQ ID NO: 81 with the following amino acid substitutions relative to the sequence of SEQ ID NO: 81: R38D, F42A and Q126S. In some embodiments, the IL-2 polypeptide comprises the sequence of SEQ ID NO: 81 with the following amino acid substitutions relative to the sequence of SEQ ID NO: 81: R38A, F42K, and N88S. In some embodiments, the IL-2 polypeptide comprises the sequence of SEQ ID NO: 81 with the following amino acid substitutions relative to the sequence of SEQ ID NO: 81: R38A, F42K, and N88A. In some embodiments, the IL-2 polypeptide comprises the sequence of SEQ ID NO: 81 with the following amino acid substitutions relative to the sequence of SEQ ID NO: 81: R38A, F42K, and N88G. In some embodiments, the IL-2 polypeptide comprises the sequence of SEQ ID NO: 81 with the following amino acid substitutions relative to the sequence of SEQ ID NO: 81: R38A, F42K, and N88R. In some embodiments, the IL-2 polypeptide comprises the sequence of SEQ ID NO: 81 with the following amino acid substitutions relative to the sequence of SEQ ID NO: 81: R38A, F42K, and N88T. In some embodiments, the IL-2 polypeptide comprises the sequence of SEQ ID NO: 81 with the following amino acid substitutions relative to the sequence of SEQ ID NO: 81: R38A, F42K, and N88D. In some embodiments, the IL-2 polypeptide comprises the sequence of SEQ ID NO: 81 with the following amino acid substitutions relative to the sequence of SEQ ID NO: 81: R38A, F42K, and V91E. In some embodiments, the IL-2 polypeptide comprises the sequence of SEQ ID NO: 81 with the following amino acid substitutions relative to the sequence of SEQ ID NO: 81: R38A, F42K, and D84H. In some embodiments, the IL-2 polypeptide comprises the sequence of SEQ ID NO: 81 with the following amino acid substitutions relative to the sequence of SEQ ID NO: 81: R38A, F42K, and D84K. In some embodiments, the IL-2 polypeptide comprises the sequence of SEQ ID NO: 81 with the following amino acid substitutions relative to the sequence of SEQ ID NO: 81: R38A, F42K, and D84R. In some embodiments, the IL-2 polypeptide comprises the sequence of SEQ ID NO: 81 with the following amino acid substitutions relative to the sequence of SEQ ID NO: 81: H16D, R38A, and F42K. In some embodiments, the IL-2 polypeptide comprises the sequence of SEQ ID NO: 81 with the following amino acid substitutions relative to the sequence of SEQ ID NO: 81: H16E, R38A, and F42K. In some embodiments, the IL-2 polypeptide comprises the sequence of SEQ ID NO: 81 with the following amino acid substitutions relative to the sequence of SEQ ID NO: 81: R38A, F42K, and Q126S. In some embodiments, the IL-2 polypeptide comprises the sequence of SEQ ID NO: 81 with the following amino acid substitutions relative to the sequence of SEQ ID NO: 81: F42A, E62Q, and N88S. In some embodiments, the IL-2 polypeptide comprises the sequence of SEQ ID NO: 81 with the following amino acid substitutions relative to the sequence of SEQ ID NO: 81: F42A, E62Q, and N88A. In some embodiments, the IL-2 polypeptide comprises the sequence of SEQ ID NO: 81 with the following amino acid substitutions relative to the sequence of SEQ ID NO: 81: F42A, E62Q, and N88G. In some embodiments, the IL-2 polypeptide comprises the sequence of SEQ ID NO: 81 with the following amino acid substitutions relative to the sequence of SEQ ID NO: 81: F42A, E62Q, and N88R. In some embodiments, the IL-2 polypeptide comprises the sequence of SEQ ID NO: 81 with the following amino acid substitutions relative to the sequence of SEQ ID NO: 81: F42A, E62Q, and N88T. In some embodiments, the IL-2 polypeptide comprises the sequence of SEQ ID NO: 81 with the following amino acid substitutions relative to the sequence of SEQ ID NO: 81: F42A, E62Q, and N88D. In some embodiments, the IL-2 polypeptide comprises the sequence of SEQ ID NO: 81 with the following amino acid substitutions relative to the sequence of SEQ ID NO: 81: F42A, E62Q, and V91E. In some embodiments, the IL-2 polypeptide comprises the sequence of SEQ ID NO: 81 with the following amino acid substitutions relative to the sequence of SEQ ID NO: 81: F42A, E62Q, and D84H. In some embodiments, the IL-2 polypeptide comprises the sequence of SEQ ID NO: 81 with the following amino acid substitutions relative to the sequence of SEQ ID NO: 81: F42A, E62Q, and D84K. In some embodiments, the IL-2 polypeptide comprises the sequence of SEQ ID NO: 81 with the following amino acid substitutions relative to the sequence of SEQ ID NO: 81: F42A, E62Q, and D84R.

In some embodiments, the IL-2 polypeptide comprises the sequence of SEQ ID NO: 81 with a further amino acid substitution relative to SEQ ID NO: 81 at position C125. In some embodiments, the IL-2 polypeptide comprises the sequence of SEQ ID NO: 81 with one of the following sets of amino acid substitutions (relative to the sequence of SEQ ID NO: 81): R38E, F42A, and C125A; R38D, F42A, and C125A; F42A, E62Q, and C125A; R38A, F42K, and C125A; R38E, F42A, N88S, and C125A; R38E, F42A, N88A, and C125A; R38E, F42A, N88G, and C125A; R38E, F42A, N88R, and C125A; R38E, F42A, N88D, and C125A; R38E, F42A, N88T, and C125A; R38E, F42A, V91E, and C125A; R38E, F42A, D84H, and C125A; R38E, F42A, D84K, and C125A; R38E, F42A, D84R, and C125A; H16D, R38E, F42A, and C125A; H16E, R38E, F42A, and C125A; R38E, F42A, C125A and Q126S; R38D, F42A, N88S, and C125A; R38D, F42A, N88A, and C125A; R38D, F42A, N88G, and C125A; R38D, F42A, N88R, and C125A; R38D, F42A, N88T, and C125A; R38D, F42A, N88D, and C125A; R38D, F42A, V91E, and C125A; R38D, F42A, D84H, and C125A; R38D, F42A, D84K, and C125A; R38D, F42A, D84R, and C125A; H16D, R38D, F42A, and C125A; H16E, R38D, F42A, and C125A; R38D, F42A, C125A, and Q126S; R38A, F42K, N88S, and C125A; R38A, F42K, N88G, and C125A; R38A, F42K, N88R, and C125A; R38A, F42K, N88T, and C125A; R38A, F42K, N88D, and C125A; R38A, F42K, N88A, and C125A; R38A, F42K, V91E, and C125A; R38A, F42K, D84H, and C125A; R38A, F42K, D84K, and C125A; R38A, F42K, D84R, and C125A; H16D, R38A, F42K, and C125A; H16E, R38A, F42K, and C125A; R38A, F42K, C125A and Q126S; F42A, E62Q, N88S, and C125A; F42A, E62Q, N88A, and C125A; F42A, E62Q, N88G, and C125A; F42A, E62Q, N88R, and C125A; F42A, E62Q, N88T, and C125A; F42A, E62Q, N88D, and C125A; F42A, E62Q, V91E, and C125A; F42A, E62Q, and D84H, and C125A; F42A, E62Q, and D84K, and C125A; F42A, E62Q, and D84R, and C125A; H16D, F42A, and E62Q, and C125A; H16E, F42A, E62Q, and C125A; F42A, E62Q, C125A and Q126S; F42A, N88S, and C125A; F42A, N88A, and C125A; F42A, N88G, and C125A; F42A, N88R, and C125A; F42A, N88T, and C125A; F42A, N88D, and C125A; F42A, V91E, and C125A; F42A, D84H, and C125A; F42A, D84K, and C125A; F42A, D84R, and C125A; H16D, F42A, and C125A; H16E, F42A, and C125A; and F42A, C125A and Q126S. In some embodiments, the IL-2 polypeptide comprises the sequence of SEQ ID NO: 81 with the following set of amino acid substitutions relative to the sequence of SEQ ID NO: 81: R38E, F42A, and C125A. In some embodiments, the IL-2 polypeptide comprises the sequence of SEQ ID NO: 81 with the following set of amino acid substitutions relative to the sequence of SEQ ID NO: 81: R38D, F42A, and C125A. In some embodiments, the IL-2 polypeptide comprises the sequence of SEQ ID NO: 81 with the following set of amino acid substitutions relative to the sequence of SEQ ID NO: 81: F42A, E62Q, and C125A. In some embodiments, the IL-2 polypeptide comprises the sequence of SEQ ID NO: 81 with the following set of amino acid substitutions relative to the sequence of SEQ ID NO: 81: R38A, F42K, and C125A. In some embodiments, the IL-2 polypeptide comprises the sequence of SEQ ID NO: 81 with the following set of amino acid substitutions relative to the sequence of SEQ ID NO: 81: R38E, F42A, N88S, and C125A. In some embodiments, the IL-2 polypeptide comprises the sequence of SEQ ID NO: 81 with the following set of amino acid substitutions relative to the sequence of SEQ ID NO: 81: R38E, F42A, N88A, and C125A. In some embodiments, the IL-2 polypeptide comprises the sequence of SEQ ID NO: 81 with the following set of amino acid substitutions relative to the sequence of SEQ ID NO: 81: R38E, F42A, N88G, and C125A. In some embodiments, the IL-2 polypeptide comprises the sequence of SEQ ID NO: 81 with the following set of amino acid substitutions relative to the sequence of SEQ ID NO: 81: R38E, F42A, N88R, and C125A. In some embodiments, the IL-2 polypeptide comprises the sequence of SEQ ID NO: 81 with the following set of amino acid substitutions relative to the sequence of SEQ ID NO: 81: R38E, F42A, N88D, and C125A. In some embodiments, the IL-2 polypeptide comprises the sequence of SEQ ID NO: 81 with the following set of amino acid substitutions relative to the sequence of SEQ ID NO: 81: R38E, F42A, N88T, and C125A. In some embodiments, the IL-2 polypeptide comprises the sequence of SEQ ID NO: 81 with the following set of amino acid substitutions relative to the sequence of SEQ ID NO: 81: R38E, F42A, V91E, and C125A. In some embodiments, the IL-2 polypeptide comprises the sequence of SEQ ID NO: 81 with the following set of amino acid substitutions relative to the sequence of SEQ ID NO: 81: R38E, F42A, D84H, and C125A. In some embodiments, the IL-2 polypeptide comprises the sequence of SEQ ID NO: 81 with the following set of amino acid substitutions relative to the sequence of SEQ ID NO: 81: R38E, F42A, D84K, and C125A. In some embodiments, the IL-2 polypeptide comprises the sequence of SEQ ID NO: 81 with the following set of amino acid substitutions relative to the sequence of SEQ ID NO: 81: R38E, F42A, D84R, and C125A. In some embodiments, the IL-2 polypeptide comprises the sequence of SEQ ID NO: 81 with the following set of amino acid substitutions relative to the sequence of SEQ ID NO: 81: H16D, R38E, F42A, and C125A. In some embodiments, the IL-2 polypeptide comprises the sequence of SEQ ID NO: 81 with the following set of amino acid substitutions relative to the sequence of SEQ ID NO: 81: H16E, R38E, F42A, and C125A. In some embodiments, the IL-2 polypeptide comprises the sequence of SEQ ID NO: 81 with the following set of amino acid substitutions relative to the sequence of SEQ ID NO: 81: R38E, F42A, C125A and Q126S. In some embodiments, the IL-2 polypeptide comprises the sequence of SEQ ID NO: 81 with the following set of amino acid substitutions relative to the sequence of SEQ ID NO: 81: R38D, F42A, N88S, and C125A. In some embodiments, the IL-2 polypeptide comprises the sequence of SEQ ID NO: 81 with the following set of amino acid substitutions relative to the sequence of SEQ ID NO: 81: R38D, F42A, N88A, and C125A. In some embodiments, the IL-2 polypeptide comprises the sequence of SEQ ID NO: 81 with the following set of amino acid substitutions relative to the sequence of SEQ ID NO: 81: R38D, F42A, N88G, and C125A. In some embodiments, the IL-2 polypeptide comprises the sequence of SEQ ID NO: 81 with the following set of amino acid substitutions relative to the sequence of SEQ ID NO: 81: R38D, F42A, N88R, and C125A. In some embodiments, the IL-2 polypeptide comprises the sequence of SEQ ID NO: 81 with the following set of amino acid substitutions relative to the sequence of SEQ ID NO: 81: R38D, F42A, N88T, and C125A. In some embodiments, the IL-2 polypeptide comprises the sequence of SEQ ID NO: 81 with the following set of amino acid substitutions relative to the sequence of SEQ ID NO: 81: R38D, F42A, N88D, and C125A. In some embodiments, the IL-2 polypeptide comprises the sequence of SEQ ID NO: 81 with the following set of amino acid substitutions relative to the sequence of SEQ ID NO: 81: R38D, F42A, V91E, and C125A. In some embodiments, the IL-2 polypeptide comprises the sequence of SEQ ID NO: 81 with the following set of amino acid substitutions relative to the sequence of SEQ ID NO: 81: R38D, F42A, D84H, and C125A. In some embodiments, the IL-2 polypeptide comprises the sequence of SEQ ID NO: 81 with the following set of amino acid substitutions relative to the sequence of SEQ ID NO: 81: R38D, F42A, D84K, and C125A. In some embodiments, the IL-2 polypeptide comprises the sequence of SEQ ID NO: 81 with the following set of amino acid substitutions relative to the sequence of SEQ ID NO: 81: R38D, F42A, D84R, and C125A. In some embodiments, the IL-2 polypeptide comprises the sequence of SEQ ID NO: 81 with the following set of amino acid substitutions relative to the sequence of SEQ ID NO: 81: H16D, R38D, F42A, and C125A. In some embodiments, the IL-2 polypeptide comprises the sequence of SEQ ID NO: 81 with the following set of amino acid substitutions relative to the sequence of SEQ ID NO: 81: H16E, R38D, F42A, and C125A. In some embodiments, the IL-2 polypeptide comprises the sequence of SEQ ID NO: 81 with the following set of amino acid substitutions relative to the sequence of SEQ ID NO: 81: R38D, F42A, C125A, and Q126S. In some embodiments, the IL-2 polypeptide comprises the sequence of SEQ ID NO: 81 with the following set of amino acid substitutions relative to the sequence of SEQ ID NO: 81: R38A, F42K, N88S, and C125A. In some embodiments, the IL-2 polypeptide comprises the sequence of SEQ ID NO: 81 with the following set of amino acid substitutions relative to the sequence of SEQ ID NO: 81: R38A, F42K, N88G, and C125A. In some embodiments, the IL-2 polypeptide comprises the sequence of SEQ ID NO: 81 with the following set of amino acid substitutions relative to the sequence of SEQ ID NO: 81: R38A, F42K, N88R, and C125A. In some embodiments, the IL-2 polypeptide comprises the sequence of SEQ ID NO: 81 with the following set of amino acid substitutions relative to the sequence of SEQ ID NO: 81: R38A, F42K, N88T, and C125A. In some embodiments, the IL-2 polypeptide comprises the sequence of SEQ ID NO: 81 with the following set of amino acid substitutions relative to the sequence of SEQ ID NO: 81: R38A, F42K, N88D, and C125A. In some embodiments, the IL-2 polypeptide comprises the sequence of SEQ ID NO: 81 with the following set of amino acid substitutions relative to the sequence of SEQ ID NO: 81: R38A, F42K, N88A, and C125A. In some embodiments, the IL-2 polypeptide comprises the sequence of SEQ ID NO: 81 with the following set of amino acid substitutions relative to the sequence of SEQ ID NO: 81: R38A, F42K, V91E, and C125A. In some embodiments, the IL-2 polypeptide comprises the sequence of SEQ ID NO: 81 with the following set of amino acid substitutions relative to the sequence of SEQ ID NO: 81: R38A, F42K, D84H, and C125A. In some embodiments, the IL-2 polypeptide comprises the sequence of SEQ ID NO: 81 with the following set of amino acid substitutions relative to the sequence of SEQ ID NO: 81: R38A, F42K, D84K, and C125A. In some embodiments, the IL-2 polypeptide comprises the sequence of SEQ ID NO: 81 with the following set of amino acid substitutions relative to the sequence of SEQ ID NO: 81: R38A, F42K, D84R, and C125A. In some embodiments, the IL-2 polypeptide comprises the sequence of SEQ ID NO: 81 with the following set of amino acid substitutions relative to the sequence of SEQ ID NO: 81: H16D, R38A, F42K, and C125A. In some embodiments, the IL-2 polypeptide comprises the sequence of SEQ ID NO: 81 with the following set of amino acid substitutions relative to the sequence of SEQ ID NO: 81: H16E, R38A, F42K, and C125A. In some embodiments, the IL-2 polypeptide comprises the sequence of SEQ ID NO: 81 with the following set of amino acid substitutions relative to the sequence of SEQ ID NO: 81: R38A, F42K, C125A and Q126S. In some embodiments, the IL-2 polypeptide comprises the sequence of SEQ ID NO: 81 with the following set of amino acid substitutions relative to the sequence of SEQ ID NO: 81: F42A, E62Q, N88S, and C125A. In some embodiments, the IL-2 polypeptide comprises the sequence of SEQ ID NO: 81 with the following set of amino acid substitutions relative to the sequence of SEQ ID NO: 81: F42A, E62Q, N88A, and C125A. In some embodiments, the IL-2 polypeptide comprises the sequence of SEQ ID NO: 81 with the following set of amino acid substitutions relative to the sequence of SEQ ID NO: 81: F42A, E62Q, N88G, and C125A. In some embodiments, the IL-2 polypeptide comprises the sequence of SEQ ID NO: 81 with the following set of amino acid substitutions relative to the sequence of SEQ ID NO: 81: F42A, E62Q, N88R, and C125A. In some embodiments, the IL-2 polypeptide comprises the sequence of SEQ ID NO: 81 with the following set of amino acid substitutions relative to the sequence of SEQ ID NO: 81: F42A, E62Q, N88T, and C125A. In some embodiments, the IL-2 polypeptide comprises the sequence of SEQ ID NO: 81 with the following set of amino acid substitutions relative to the sequence of SEQ ID NO: 81: F42A, E62Q, N88D, and C125A. In some embodiments, the IL-2 polypeptide comprises the sequence of SEQ ID NO: 81 with the following set of amino acid substitutions relative to the sequence of SEQ ID NO: 81: F42A, E62Q, V91E, and C125A. In some embodiments, the IL-2 polypeptide comprises the sequence of SEQ ID NO: 81 with the following set of amino acid substitutions relative to the sequence of SEQ ID NO: 81: F42A, E62Q, and D84H, and C125A. In some embodiments, the IL-2 polypeptide comprises the sequence of SEQ ID NO: 81 with the following set of amino acid substitutions relative to the sequence of SEQ ID NO: 81: F42A, E62Q, and D84K, and C125A. In some embodiments, the IL-2 polypeptide comprises the sequence of SEQ ID NO: 81 with the following set of amino acid substitutions relative to the sequence of SEQ ID NO: 81: F42A, E62Q, and D84R, and C125A. In some embodiments, the IL-2 polypeptide comprises the sequence of SEQ ID NO: 81 with the following set of amino acid substitutions relative to the sequence of SEQ ID NO: 81: H16D, F42A, and E62Q, and C125A. In some embodiments, the IL-2 polypeptide comprises the sequence of SEQ ID NO: 81 with the following set of amino acid substitutions relative to the sequence of SEQ ID NO: 81: H16E, F42A, E62Q, and C125A. In some embodiments, the IL-2 polypeptide comprises the sequence of SEQ ID NO: 81 with the following set of amino acid substitutions relative to the sequence of SEQ ID NO: 81: F42A, E62Q, C125A and Q126S. In some embodiments, the IL-2 polypeptide comprises the sequence of SEQ ID NO: 81 with the following set of amino acid substitutions relative to the sequence of SEQ ID NO: 81: F42A, N88S, and C125A. In some embodiments, the IL-2 polypeptide comprises the sequence of SEQ ID NO: 81 with the following set of amino acid substitutions relative to the sequence of SEQ ID NO: 81: F42A, N88A, and C125A. In some embodiments, the IL-2 polypeptide comprises the sequence of SEQ ID NO: 81 with the following set of amino acid substitutions relative to the sequence of SEQ ID NO: 81: F42A, N88G, and C125A. In some embodiments, the IL-2 polypeptide comprises the sequence of SEQ ID NO: 81 with the following set of amino acid substitutions relative to the sequence of SEQ ID NO: 81: F42A, N88R, and C125A. In some embodiments, the IL-2 polypeptide comprises the sequence of SEQ ID NO: 81 with the following set of amino acid substitutions relative to the sequence of SEQ ID NO: 81: F42A, N88T, and C125A. In some embodiments, the IL-2 polypeptide comprises the sequence of SEQ ID NO: 81 with the following set of amino acid substitutions relative to the sequence of SEQ ID NO: 81: F42A, N88D, and C125A; F42A, V91E, and C125A. In some embodiments, the IL-2 polypeptide comprises the sequence of SEQ ID NO: 81 with the following set of amino acid substitutions relative to the sequence of SEQ ID NO: 81: F42A, D84H, and C125A. In some embodiments, the IL-2 polypeptide comprises the sequence of SEQ ID NO: 81 with the following set of amino acid substitutions relative to the sequence of SEQ ID NO: 81: F42A, D84K, and C125A. In some embodiments, the IL-2 polypeptide comprises the sequence of SEQ ID NO: 81 with the following set of amino acid substitutions relative to the sequence of SEQ ID NO: 81: F42A, D84R, and C125A. In some embodiments, the IL-2 polypeptide comprises the sequence of SEQ ID NO: 81 with the following set of amino acid substitutions relative to the sequence of SEQ ID NO: 81: H16D, F42A, and C125A. In some embodiments, the IL-2 polypeptide comprises the sequence of SEQ ID NO: 81 with the following set of amino acid substitutions relative to the sequence of SEQ ID NO: 81: H16E, F42A, and C125A. In some embodiments, the IL-2 polypeptide comprises the sequence of SEQ ID NO: 81 with the following set of amino acid substitutions relative to the sequence of SEQ ID NO: 81: F42A, C125A and Q126S.

In some embodiments, the IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISAINVIVLELKGSETTFMCEYADETATIVE FLNRWITFAQSIISTLT (SEQ ID NO: 80). In some embodiments, the IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISNINVIVLELKGSETTFMCEYADETATIVE FLNRWITFCQSIISTLT (SEQ ID NO: 85). In some embodiments, the IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISNINVIVLELKGSETTFMCEYADETATIV EFLNRWITFCQSIISTLT (SEQ ID NO: 86). In some embodiments, the IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISNINVIVLELKGSETTFMCEYADETATIVE FLNRWITFCQSIISTLT (SEQ ID NO: 87). In some embodiments, the IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISNINVIVLELKGSETTFMCEYADETATIV EFLNRWITFCQSIISTLT (SEQ ID NO: 88). In some embodiments, the IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISSINVIVLELKGSETTFMCEYADETATIVE FLNRWITFCQSIISTLT (SEQ ID NO: 89). In some embodiments, the IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISAINVIVLELKGSETTFMCEYADETATIVE FLNRWITFCQSIISTLT (SEQ ID NO: 90). In some embodiments, the IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISNINEIVLELKGSETTFMCEYADETATIVE FLNRWITFCQSIISTLT (SEQ ID NO: 91). In some embodiments, the IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRHLISNINVIVLELKGSETTFMCEYADETATIVE FLNRWITFCQSIISTLT (SEQ ID NO: 92). In some embodiments, the IL-2 polypeptide comprises the amino acid sequence of APTSSSTKKTQLQLEDLLLDLQMILNGINNYKNPKLTEMLTAKFYMPKKATELKHLQ CLEEELKPLEEVLNLAQSKNFHLRPRDLISNINVIVLELKGSETTFMCEYADETATIVE FLNRWITFCQSIISTLT (SEQ ID NO: 93). In some embodiments, the IL-2 polypeptide comprises the amino acid sequence of APTSSSTKKTQLQLEELLLDLQMILNGINNYKNPKLTEMLTAKFYMPKKATELKHLQ CLEEELKPLEEVLNLAQSKNFHLRPRDLISNINVIVLELKGSETTFMCEYADETATIVE FLNRWITFCQSIISTLT (SEQ ID NO: 94). In some embodiments, the IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISNINVIVLELKGSETTFMCEYADETATIVE FLNRWITFCSSIISTLT (SEQ ID NO: 95). In some embodiments, the IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISSINVIVLELKGSETTFMCEYADETATIV EFLNRWITFCQSIISTLT (SEQ ID NO: 96). In some embodiments, the IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISAINVIVLELKGSETTFMCEYADETATIV EFLNRWITFCQSIISTLT (SEQ ID NO: 97). In some embodiments, the IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISNINEIVLELKGSETTFMCEYADETATIV EFLNRWITFCQSIISTLT (SEQ ID NO: 98). In some embodiments, the IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRHLISNINVIVLELKGSETTFMCEYADETATIV EFLNRWITFCQSIISTLT (SEQ ID NO: 99). In some embodiments, the IL-2 polypeptide comprises the amino acid sequence of APTSSSTKKTQLQLEDLLLDLQMILNGINNYKNPKLTDMLTAKFYMPKKATELKHL QCLEEELKPLEEVLNLAQSKNFHLRPRDLISNINVIVLELKGSETTFMCEYADETATIV EFLNRWITFCQSIISTLT (SEQ ID NO: 100). In some embodiments, the IL-2 polypeptide comprises the amino acid sequence of APTSSSTKKTQLQLEELLLDLQMILNGINNYKNPKLTDMLTAKFYMPKKATELKHLQ CLEEELKPLEEVLNLAQSKNFHLRPRDLISNINVIVLELKGSETTFMCEYADETATIVE FLNRWITFCQSIISTLT (SEQ ID NO: 101). In some embodiments, the IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISNINVIVLELKGSETTFMCEYADETATIV EFLNRWITFCSSIISTLT (SEQ ID NO: 102). In some embodiments, the IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISSINVIVLELKGSETTFMCEYADETATIV EFLNRWITFCQSIISTLT (SEQ ID NO: 103). In some embodiments, the IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISAINVIVLELKGSETTFMCEYADETATIV EFLNRWITFCQSIISTLT (SEQ ID NO: 104). In some embodiments, the IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISNINEIVLELKGSETTFMCEYADETATIV EFLNRWITFCQSIISTLT (SEQ ID NO: 105). In some embodiments, the IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRHLISNINVIVLELKGSETTFMCEYADETATIV EFLNRWITFCQSIISTLT (SEQ ID NO: 106). In some embodiments, the IL-2 polypeptide comprises the amino acid sequence of APTSSSTKKTQLQLEDLLLDLQMILNGINNYKNPKLTAMLTKKFYMPKKATELKHL QCLEEELKPLEEVLNLAQSKNFHLRPRDLISNINVIVLELKGSETTFMCEYADETATIV EFLNRWITFCQSIISTLT (SEQ ID NO: 107). In some embodiments, the IL-2 polypeptide comprises the amino acid sequence of APTSSSTKKTQLQLEELLLDLQMILNGINNYKNPKLTAMLTKKFYMPKKATELKHLQ CLEEELKPLEEVLNLAQSKNFHLRPRDLISNINVIVLELKGSETTFMCEYADETATIVE FLNRWITFCQSIISTLT (SEQ ID NO: 108). In some embodiments, the IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISNINVIVLELKGSETTFMCEYADETATIV EFLNRWITFCSSIISTLT (SEQ ID NO: 109). In some embodiments, the IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISSINVIVLELKGSETTFMCEYADETATIVE FLNRWITFCQSIISTLT (SEQ ID NO: 110). In some embodiments, the IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISAINVIVLELKGSETTFMCEYADETATIVE FLNRWITFCQSIISTLT (SEQ ID NO: 111). In some embodiments, the IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISNINEIVLELKGSETTFMCEYADETATIVE FLNRWITFCQSIISTLT (SEQ ID NO: 112). In some embodiments, the IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRHLISNINVIVLELKGSETTFMCEYADETATIVE FLNRWITFCQSIISTLT (SEQ ID NO: 113). In some embodiments, the IL-2 polypeptide comprises the amino acid sequence of APTSSSTKKTQLQLEDLLLDLQMILNGINNYKNPKLTRMLTAKFYMPKKATELKHLQ CLEEQLKPLEEVLNLAQSKNFHLRPRDLISNINVIVLELKGSETTFMCEYADETATIVE FLNRWITFCQSIISTLT (SEQ ID NO: 114). In some embodiments, the IL-2 polypeptide comprises the amino acid sequence of APTSSSTKKTQLQLEELLLDLQMILNGINNYKNPKLTRMLTAKFYMPKKATELKHLQ CLEEQLKPLEEVLNLAQSKNFHLRPRDLISNINVIVLELKGSETTFMCEYADETATIVE FLNRWITFCQSIISTLT (SEQ ID NO: 115). In some embodiments, the IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISNINVIVLELKGSETTFMCEYADETATIVE FLNRWITFCSSIISTLT (SEQ ID NO: 116). In some embodiments, the IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISNINVIVLELKGSETTFMCEYADETATIVE FLNRWITFAQSIISTLT (SEQ ID NO: 117). In some embodiments, the IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISNINVIVLELKGSETTFMCEYADETATIV EFLNRWITFAQSIISTLT (SEQ ID NO: 118). In some embodiments, the IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISNINVIVLELKGSETTFMCEYADETATIVE FLNRWITFAQSIISTLT (SEQ ID NO: 119). In some embodiments, the IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISNINVIVLELKGSETTFMCEYADETATIV EFLNRWITFAQSIISTLT (SEQ ID NO: 120). In some embodiments, the IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISSINVIVLELKGSETTFMCEYADETATIVE FLNRWITFAQSIISTLT (SEQ ID NO: 121). In some embodiments, the IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISAINVIVLELKGSETTFMCEYADETATIVE FLNRWITFAQSIISTLT (SEQ ID NO: 122). In some embodiments, the IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISNINEIVLELKGSETTFMCEYADETATIVE FLNRWITFAQSIISTLT (SEQ ID NO: 123). In some embodiments, the IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRHLISNINVIVLELKGSETTFMCEYADETATIVE FLNRWITFAQSIISTLT (SEQ ID NO: 124). In some embodiments, the IL-2 polypeptide comprises the amino acid sequence of APTSSSTKKTQLQLEDLLLDLQMILNGINNYKNPKLTEMLTAKFYMPKKATELKHLQ CLEEELKPLEEVLNLAQSKNFHLRPRDLISNINVIVLELKGSETTFMCEYADETATIVE FLNRWITFAQSIISTLT (SEQ ID NO: 125). In some embodiments, the IL-2 polypeptide comprises the amino acid sequence of APTSSSTKKTQLQLEELLLDLQMILNGINNYKNPKLTEMLTAKFYMPKKATELKHLQ CLEEELKPLEEVLNLAQSKNFHLRPRDLISNINVIVLELKGSETTFMCEYADETATIVE FLNRWITFAQSIISTLT (SEQ ID NO: 126). In some embodiments, the IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISNINVIVLELKGSETTFMCEYADETATIVE FLNRWITFASSIISTLT (SEQ ID NO: 127). In some embodiments, the IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISSINVIVLELKGSETTFMCEYADETATIV EFLNRWITFAQSIISTLT (SEQ ID NO: 128). In some embodiments, the IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISAINVIVLELKGSETTFMCEYADETATIV EFLNRWITFAQSIISTLT (SEQ ID NO: 129). In some embodiments, the IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISNINEIVLELKGSETTFMCEYADETATIV EFLNRWITFAQSIISTLT (SEQ ID NO: 130). In some embodiments, the IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRHLISNINVIVLELKGSETTFMCEYADETATIV EFLNRWITFAQSIISTLT (SEQ ID NO: 131). In some embodiments, the IL-2 polypeptide comprises the amino acid sequence of APTSSSTKKTQLQLEDLLLDLQMILNGINNYKNPKLTDMLTAKFYMPKKATELKHL QCLEEELKPLEEVLNLAQSKNFHLRPRDLISNINVIVLELKGSETTFMCEYADETATIV EFLNRWITFAQSIISTLT (SEQ ID NO: 132). In some embodiments, the IL-2 polypeptide comprises the amino acid sequence of APTSSSTKKTQLQLEELLLDLQMILNGINNYKNPKLTDMLTAKFYMPKKATELKHLQ CLEEELKPLEEVLNLAQSKNFHLRPRDLISNINVIVLELKGSETTFMCEYADETATIVE FLNRWITFAQSIISTLT (SEQ ID NO: 133). In some embodiments, the IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISNINVIVLELKGSETTFMCEYADETATIV EFLNRWITFASSIISTLT (SEQ ID NO: 134). In some embodiments, the IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISSINVIVLELKGSETTFMCEYADETATIV EFLNRWITFAQSIISTLT (SEQ ID NO: 135). In some embodiments, the IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISAINVIVLELKGSETTFMCEYADETATIV EFLNRWITFAQSIISTLT (SEQ ID NO: 136). In some embodiments, the IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISNINEIVLELKGSETTFMCEYADETATIV EFLNRWITFAQSIISTLT (SEQ ID NO: 137). In some embodiments, the IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRHLISNINVIVLELKGSETTFMCEYADETATIV EFLNRWITFAQSIISTLT (SEQ ID NO: 138). In some embodiments, the IL-2 polypeptide comprises the amino acid sequence of APTSSSTKKTQLQLEDLLLDLQMILNGINNYKNPKLTAMLTKKFYMPKKATELKHL QCLEEELKPLEEVLNLAQSKNFHLRPRDLISNINVIVLELKGSETTFMCEYADETATIV EFLNRWITFAQSIISTLT (SEQ ID NO: 139). In some embodiments, the IL-2 polypeptide comprises the amino acid sequence of APTSSSTKKTQLQLEELLLDLQMILNGINNYKNPKLTAMLTKKFYMPKKATELKHLQ CLEEELKPLEEVLNLAQSKNFHLRPRDLISNINVIVLELKGSETTFMCEYADETATIVE FLNRWITFAQSIISTLT (SEQ ID NO: 140). In some embodiments, the IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISNINVIVLELKGSETTFMCEYADETATIV EFLNRWITFASSIISTLT (SEQ ID NO: 141). In some embodiments, the IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISSINVIVLELKGSETTFMCEYADETATIVE FLNRWITFAQSIISTLT (SEQ ID NO: 142). In some embodiments, the IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISAINVIVLELKGSETTFMCEYADETATIVE FLNRWITFAQSIISTLT (SEQ ID NO: 143). In some embodiments, the IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISNINEIVLELKGSETTFMCEYADETATIVE FLNRWITFAQSIISTLT (SEQ ID NO: 144). In some embodiments, the IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRHLISNINVIVLELKGSETTFMCEYADETATIVE FLNRWITFAQSIISTLT (SEQ ID NO: 145). In some embodiments, the IL-2 polypeptide comprises the amino acid sequence of APTSSSTKKTQLQLEDLLLDLQMILNGINNYKNPKLTRMLTAKFYMPKKATELKHLQ CLEEQLKPLEEVLNLAQSKNFHLRPRDLISNINVIVLELKGSETTFMCEYADETATIVE FLNRWITFAQSIISTLT (SEQ ID NO: 146). In some embodiments, the IL-2 polypeptide comprises the amino acid sequence of APTSSSTKKTQLQLEELLLDLQMILNGINNYKNPKLTRMLTAKFYMPKKATELKHLQ CLEEQLKPLEEVLNLAQSKNFHLRPRDLISNINVIVLELKGSETTFMCEYADETATIVE FLNRWITFAQSIISTLT (SEQ ID NO: 147). In some embodiments, the IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISNINVIVLELKGSETTFMCEYADETATIVE FLNRWITFASSIISTLT (SEQ ID NO: 148). In some embodiments, the IL-2 polypeptide comprises the amino acid sequence of APTSSSTKKTQLQLEHLLLDLQMILNGINNYKNPKLTRMLTAKFYMPKKATELKHLQ CLEEELKPLEEVLNLAQSKNFHLRPRDLISSINVIVLELKGSETTFMCEYADETATIVE FLNRWITFAQSIISTLT (SEQ ID NO: 149). In some embodiments, the IL-2 polypeptide comprises the amino acid sequence of APTSSSTKKTQLQLEHLLLDLQMILNGINNYKNPKLTRMLTAKFYMPKKATELKHLQ CLEEELKPLEEVLNLAQSKNFHLRPRDLISAINVIVLELKGSETTFMCEYADETATIVE FLNRWITFAQSIISTLT (SEQ ID NO: 150). In some embodiments, the IL-2 polypeptide comprises the amino acid sequence of APTSSSTKKTQLQLEHLLLDLQMILNGINNYKNPKLTRMLTAKFYMPKKATELKHLQ CLEEELKPLEEVLNLAQSKNFHLRPRDLISNINEIVLELKGSETTFMCEYADETATIVE FLNRWITFAQSIISTLT (SEQ ID NO: 151). In some embodiments, the IL-2 polypeptide comprises the amino acid sequence of APTSSSTKKTQLQLEHLLLDLQMILNGINNYKNPKLTRMLTAKFYMPKKATELKHLQ CLEEELKPLEEVLNLAQSKNFHLRPRHLISNINVIVLELKGSETTFMCEYADETATIVE FLNRWITFAQSIISTLT (SEQ ID NO: 152). In some embodiments, the IL-2 polypeptide comprises the amino acid sequence of APTSSSTKKTQLQLEDLLLDLQMILNGINNYKNPKLTRMLTAKFYMPKKATELKHLQ CLEEELKPLEEVLNLAQSKNFHLRPRDLISNINVIVLELKGSETTFMCEYADETATIVE FLNRWITFAQSIISTLT (SEQ ID NO: 153). In some embodiments, the IL-2 polypeptide comprises the amino acid sequence of APTSSSTKKTQLQLEELLLDLQMILNGINNYKNPKLTRMLTAKFYMPKKATELKHLQ CLEEELKPLEEVLNLAQSKNFHLRPRDLISNINVIVLELKGSETTFMCEYADETATIVE FLNRWITFAQSIISTLT (SEQ ID NO: 154). In some embodiments, the IL-2 polypeptide comprises the amino acid sequence of APTSSSTKKTQLQLEHLLLDLQMILNGINNYKNPKLTRMLTAKFYMPKKATELKHLQ CLEEELKPLEEVLNLAQSKNFHLRPRDLISNINVIVLELKGSETTFMCEYADETATIVE FLNRWITFASSIISTLT (SEQ ID NO: 155). In some embodiments, the IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISGINVIVLELKGSETTFMCEYADETATIVE FLNRWITFCQSIISTLT (SEQ ID NO: 190). In some embodiments, the IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRKLISNINVIVLELKGSETTFMCEYADETATIVE FLNRWITFCQSIISTLT (SEQ ID NO: 191). In some embodiments, the IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRRLISNINVIVLELKGSETTFMCEYADETATIVE FLNRWITFCQSIISTLT (SEQ ID NO: 192). In some embodiments, the IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISGINVIVLELKGSETTFMCEYADETATIV EFLNRWITFCQSIISTLT (SEQ ID NO: 193). In some embodiments, the IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRKLISNINVIVLELKGSETTFMCEYADETATIV EFLNRWITFCQSIISTLT (SEQ ID NO: 194). In some embodiments, the IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRRLISNINVIVLELKGSETTFMCEYADETATIV EFLNRWITFCQSIISTLT (SEQ ID NO: 195). In some embodiments, the IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISGINVIVLELKGSETTFMCEYADETATIV EFLNRWITFCQSIISTLT (SEQ ID NO: 196). In some embodiments, the IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRKLISNINVIVLELKGSETTFMCEYADETATIV EFLNRWITFCQSIISTLT (SEQ ID NO: 197). In some embodiments, the IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRRLISNINVIVLELKGSETTFMCEYADETATIV EFLNRWITFCQSIISTLT (SEQ ID NO: 198). In some embodiments, the IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISGINVIVLELKGSETTFMCEYADETATIVE FLNRWITFCQSIISTLT (SEQ ID NO: 199). In some embodiments, the IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRKLISNINVIVLELKGSETTFMCEYADETATIVE FLNRWITFCQSIISTLT (SEQ ID NO: 200). In some embodiments, the IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRRLISNINVIVLELKGSETTFMCEYADETATIVE FLNRWITFCQSIISTLT (SEQ ID NO: 201). In some embodiments, the IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISGINVIVLELKGSETTFMCEYADETATIVE FLNRWITFAQSIISTLT (SEQ ID NO: 202). In some embodiments, the IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRKLISNINVIVLELKGSETTFMCEYADETATIVE FLNRWITFAQSIISTLT (SEQ ID NO: 203). In some embodiments, the IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRRLISNINVIVLELKGSETTFMCEYADETATIVE FLNRWITFAQSIISTLT (SEQ ID NO: 204). In some embodiments, the IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISGINVIVLELKGSETTFMCEYADETATIV EFLNRWITFAQSIISTLT (SEQ ID NO: 205). In some embodiments, the IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRKLISNINVIVLELKGSETTFMCEYADETATIV EFLNRWITFAQSIISTLT (SEQ ID NO: 206). In some embodiments, the IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRRLISNINVIVLELKGSETTFMCEYADETATIV EFLNRWITFAQSIISTLT (SEQ ID NO: 207). In some embodiments, the IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISGINVIVLELKGSETTFMCEYADETATIV EFLNRWITFAQSIISTLT (SEQ ID NO: 208). In some embodiments, the IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRKLISNINVIVLELKGSETTFMCEYADETATIV EFLNRWITFAQSIISTLT (SEQ ID NO: 209). In some embodiments, the IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRRLISNINVIVLELKGSETTFMCEYADETATIV EFLNRWITFAQSIISTLT (SEQ ID NO: 210). In some embodiments, the IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISGINVIVLELKGSETTFMCEYADETATIVE FLNRWITFAQSIISTLT (SEQ ID NO: 211). In some embodiments, the IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRKLISNINVIVLELKGSETTFMCEYADETATIVE FLNRWITFAQSIISTLT (SEQ ID NO: 212). In some embodiments, the IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRRLISNINVIVLELKGSETTFMCEYADETATIVE FLNRWITFAQSIISTLT (SEQ ID NO: 213). In some embodiments, the IL-2 polypeptide comprises the amino acid sequence of APTSSSTKKTQLQLEHLLLDLQMILNGINNYKNPKLTRMLTAKFYMPKKATELKHLQ CLEEELKPLEEVLNLAQSKNFHLRPRDLISGINVIVLELKGSETTFMCEYADETATIVE FLNRWITFAQSIISTLT (SEQ ID NO: 214). In some embodiments, the IL-2 polypeptide comprises the amino acid sequence of APTSSSTKKTQLQLEHLLLDLQMILNGINNYKNPKLTRMLTAKFYMPKKATELKHLQ CLEEELKPLEEVLNLAQSKNFHLRPRKLISNINVIVLELKGSETTFMCEYADETATIVE FLNRWITFAQSIISTLT (SEQ ID NO: 215). In some embodiments, the IL-2 polypeptide comprises the amino acid sequence of APTSSSTKKTQLQLEHLLLDLQMILNGINNYKNPKLTRMLTAKFYMPKKATELKHLQ CLEEELKPLEEVLNLAQSKNFHLRPRRLISNINVIVLELKGSETTFMCEYADETATIVE FLNRWITFAQSIISTLT (SEQ ID NO: 216). In some embodiments, the IL-2 polypeptide comprises the amino acid sequence of APTSSSTKKTQLQLEELLLDLQMILNGINNYKNPKLTEMLTAKFYMPKKATELKHLQ CLEEELKPLEEVLNLAQSKNFHLRPRDLISNINVIVLELKGSETTFMCEYADETATIVE FLNRWITFAQSIISTLT (SEQ ID NO: 297). In some embodiments, the IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISDINVIVLELKGSETTFMCEYADETATIVE FLNRWITFAQSIISTLT (SEQ ID NO: 354). In some embodiments, the IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISTINVIVLELKGSETTFMCEYADETATIVE FLNRWITFAQSIISTLT (SEQ ID NO: 355). In some embodiments, the IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISRINVIVLELKGSETTFMCEYADETATIVE FLNRWITFAQSIISTLT (SEQ ID NO: 356). In some embodiments, the IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISRINVIVLELKGSETTFMCEYADETATIVE FLNRWITFCQSIISTLT (SEQ ID NO: 357). In some embodiments, the IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISTINVIVLELKGSETTFMCEYADETATIVE FLNRWITFCQSIISTLT (SEQ ID NO: 358). In some embodiments, the IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISDINVIVLELKGSETTFMCEYADETATIVE FLNRWITFCQSIISTLT (SEQ ID NO: 359). In some embodiments, the IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISRINVIVLELKGSETTFMCEYADETATIV EFLNRWITFCQSIISTLT (SEQ ID NO: 360). In some embodiments, the IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISTINVIVLELKGSETTFMCEYADETATIV EFLNRWITFCQSIISTLT (SEQ ID NO: 361). In some embodiments, the IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISDINVIVLELKGSETTFMCEYADETATIV EFLNRWITFCQSIISTLT (SEQ ID NO: 362). In some embodiments, the IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISRINVIVLELKGSETTFMCEYADETATIV EFLNRWITFCQSIISTLT (SEQ ID NO: 363). In some embodiments, the IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISTINVIVLELKGSETTFMCEYADETATIV EFLNRWITFCQSIISTLT (SEQ ID NO: 364). In some embodiments, the IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISDINVIVLELKGSETTFMCEYADETATIV EFLNRWITFCQSIISTLT (SEQ ID NO: 365). In some embodiments, the IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISRINVIVLELKGSETTFMCEYADETATIVE FLNRWITFCQSIISTLT (SEQ ID NO: 366). In some embodiments, the IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISTINVIVLELKGSETTFMCEYADETATIVE FLNRWITFCQSIISTLT (SEQ ID NO: 367). In some embodiments, the IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISDINVIVLELKGSETTFMCEYADETATIVE FLNRWITFCQSIISTLT (SEQ ID NO: 368). In some embodiments, the IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISRINVIVLELKGSETTFMCEYADETATIVE FLNRWITFAQSIISTLT (SEQ ID NO: 369). In some embodiments, the IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISTINVIVLELKGSETTFMCEYADETATIVE FLNRWITFAQSIISTLT (SEQ ID NO: 370). In some embodiments, the IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISDINVIVLELKGSETTFMCEYADETATIVE FLNRWITFAQSIISTLT (SEQ ID NO: 371). In some embodiments, the IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISRINVIVLELKGSETTFMCEYADETATIV EFLNRWITFAQSIISTLT (SEQ ID NO: 372). In some embodiments, the IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISTINVIVLELKGSETTFMCEYADETATIV EFLNRWITFAQSIISTLT (SEQ ID NO: 373). In some embodiments, the IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISDINVIVLELKGSETTFMCEYADETATIV EFLNRWITFAQSIISTLT (SEQ ID NO: 374). In some embodiments, the IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISRINVIVLELKGSETTFMCEYADETATIV EFLNRWITFAQSIISTLT (SEQ ID NO: 375). In some embodiments, the IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISTINVIVLELKGSETTFMCEYADETATIV EFLNRWITFAQSIISTLT (SEQ ID NO: 376). In some embodiments, the IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISDINVIVLELKGSETTFMCEYADETATIV EFLNRWITFAQSIISTLT (SEQ ID NO: 377). In some embodiments, the IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISRINVIVLELKGSETTFMCEYADETATIVE FLNRWITFAQSIISTLT (SEQ ID NO: 378). In some embodiments, the IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISTINVIVLELKGSETTFMCEYADETATIVE FLNRWITFAQSIISTLT (SEQ ID NO: 379). In some embodiments, the IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISDINVIVLELKGSETTFMCEYADETATIVE FLNRWITFAQSIISTLT (SEQ ID NO: 380). In some embodiments, the IL-2 polypeptide comprises the amino acid sequence of APTSSSTKKTQLQLEHLLLDLQMILNGINNYKNPKLTRMLTAKFYMPKKATELKHLQ CLEEELKPLEEVLNLAQSKNFHLRPRDLISRINVIVLELKGSETTFMCEYADETATIVE FLNRWITFAQSIISTLT (SEQ ID NO: 381). In some embodiments, the IL-2 polypeptide comprises the amino acid sequence of APTSSSTKKTQLQLEHLLLDLQMILNGINNYKNPKLTRMLTAKFYMPKKATELKHLQ CLEEELKPLEEVLNLAQSKNFHLRPRDLISTINVIVLELKGSETTFMCEYADETATIVE FLNRWITFAQSIISTLT (SEQ ID NO: 382). In some embodiments, the IL-2 polypeptide comprises the amino acid sequence of APTSSSTKKTQLQLEHLLLDLQMILNGINNYKNPKLTRMLTAKFYMPKKATELKHLQ CLEEELKPLEEVLNLAQSKNFHLRPRDLISDINVIVLELKGSETTFMCEYADETATIVE FLNRWITFAQSIISTLT (SEQ ID NO: 383). In some embodiments, the IL-2 polypeptide comprises the amino acid sequence of an IL-2 polypeptide listed in Table 6.

In some embodiments, the IL-2 polypeptide comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequence set forth in SEQ ID NOs: 80, 85-155, 190-216, 297, or 354-383. In some embodiments, the IL-2 polypeptide comprises an amino acid sequence having at least 80% identity to any one of the amino acid sequence set forth in SEQ ID NOs: 80, 85-155, 190-216, 297, or 354-383. In some embodiments, the IL-2 polypeptide comprises an amino acid sequence having at least 85% identity to any one of the amino acid sequence set forth in SEQ ID NOs: 80, 85-155, 190-216, 297, or 354-383. In some embodiments, the IL-2 polypeptide comprises an amino acid sequence having at least 90% identity to any one of the amino acid sequence set forth in SEQ ID NOs: 80, 85-155, 190-216, 297, or 354-383. In some embodiments, the IL-2 polypeptide comprises an amino acid sequence having at least 95% identity to any one of the amino acid sequence set forth in SEQ ID NOs: 80, 85-155, 190-216, 297, or 354-383. In some embodiments, the IL-2 polypeptide comprises an amino acid sequence having at least 96% identity to any one of the amino acid sequence set forth in SEQ ID NOs: 80, 85-155, 190-216, 297, or 354-383. In some embodiments, the IL-2 polypeptide comprises an amino acid sequence having at least 97% identity to any one of the amino acid sequence set forth in SEQ ID NOs: 80, 85-155, 190-216, 297, or 354-383. In some embodiments, the IL-2 polypeptide comprises an amino acid sequence having at least 98% identity to any one of the amino acid sequence set forth in SEQ ID NOs: 80, 85-155, 190-216, 297, or 354-383. In some embodiments, the IL-2 polypeptide comprises an amino acid sequence having at least 99% identity to any one of the amino acid sequence set forth in SEQ ID NOs: 80, 85-155, 190-216, 297, or 354-383. In some embodiments, the IL-2 polypeptide comprises an amino acid sequence having 100% identity to any one of the amino acid sequence set forth in SEQ ID NOs: 80, 85-155, 190-216, 297, or 354-383.

TABLE 6
Exemplary IL-2 polypeptide sequences

| | | SEQ |
|---|---|---|
| IL-2 | | ID |
| ID | Sequence | NO |
| m1 | APTSSSTKKTQLQLEHLLLDLQMILNGINNYKNPKLTEMLTAKFYMPKKATEL | 80 |
| | KHLQCLEEELKPLEEVLNLAQSKNFHLRPRDLISAINVIVLELKGSETTFMCEYA | |
| | DETATIVEFLNRWITFAQSIISTLT | |
| m2 | APTSSSTKKTQLQLEHLLLDLQMILNGINNYKNPKLTEMLTAKFYMPKKATEL | 121 |
| | KHLQCLEEELKPLEEVLNLAQSKNFHLRPRDLISSINVIVLELKGSETTFMCEYA | |
| | DETATIVEFLNRWITFAQSIISTLT | |
| m3 | APTSSSTKKTQLQLEDLLLDLQMILNGINNYKNPKLTEMLTAKFYMPKKATEL | 125 |
| | KHLQCLEEELKPLEEVLNLAQSKNFHLRPRDLISNINVIVLELKGSETTFMCEYA | |
| | DETATIVEFLNRWITFAQSIISTLT | |
| m4 | APTSSSTKKTQLQLEELLLDLQMILNGINNYKNPKLTEMLTAKFYMPKKATELK | 297 |
| | HLQCLEEELKPLEEVLNLAQSKNFHLRPRDLISNINVIVLELKGSETTFMCEYAD | |
| | ETATIVEFLNRWITFAQSIISTLT | |
| m5 | APTSSSTKKTQLQLEHLLLDLQMILNGINNYKNPKLTEMLTAKFYMPKKATEL | 202 |
| | KHLQCLEEELKPLEEVLNLAQSKNFHLRPRDLISGINVIVLELKGSETTFMCEYA | |
| | DETATIVEFLNRWITFAQSIISTLT | |
| m6 | APTSSSTKKTQLQLEHLLLDLQMILNGINNYKNPKLTEMLTAKFYMPKKATEL | 117 |
| | KHLQCLEEELKPLEEVLNLAQSKNFHLRPRDLISNINVIVLELKGSETTFMCEYA | |
| | DETATIVEFLNRWITFAQSIISTLT | |
| m7 | APTSSSTKKTQLQLEHLLLDLQMILNGINNYKNPKLTEMLTAKFYMPKKATEL | 123 |
| | KHLQCLEEELKPLEEVLNLAQSKNFHLRPRDLISNINEIVLELKGSETTFMCEYA | |
| | DETATIVEFLNRWITFAQSIISTLT | |
| m8 | APTSSSTKKTQLQLEHLLLDLQMILNGINNYKNPKLTEMLTAKFYMPKKATEL | 124 |
| | KHLQCLEEELKPLEEVLNLAQSKNFHLRPRHLISNINVIVLELKGSETTFMCEYA | |
| | DETATIVEFLNRWITFAQSIISTLT | |
| m9 | APTSSSTKKTQLQLEHLLLDLQMILNGINNYKNPKLTEMLTAKFYMPKKATEL | 203 |
| | KHLQCLEEELKPLEEVLNLAQSKNFHLRPRKLISNINVIVLELKGSETTFMCEYA | |
| | DETATIVEFLNRWITFAQSIISTLT | |
| m10 | APTSSSTKKTQLQLEHLLLDLQMILNGINNYKNPKLTEMLTAKFYMPKKATEL | 204 |
| | KHLQCLEEELKPLEEVLNLAQSKNFHLRPRRLISNINVIVLELKGSETTFMCEYA | |
| | DETATIVEFLNRWITFAQSIISTLT | |
| m11 | APTSSSTKKTQLQLEHLLLDLQMILNGINNYKNPKLTEMLTAKFYMPKKATEL | 354 |
| | KHLQCLEEELKPLEEVLNLAQSKNFHLRPRDLISDINVIVLELKGSETTFMCEYA | |
| | DETATIVEFLNRWITFAQSIISTLT | |
| m12 | APTSSSTKKTQLQLEHLLLDLQMILNGINNYKNPKLTEMLTAKFYMPKKATEL | 355 |
| | KHLQCLEEELKPLEEVLNLAQSKNFHLRPRDLISTINVIVLELKGSETTFMCEYA | |
| | DETATIVEFLNRWITFAQSIISTLT | |
| m13 | APTSSSTKKTQLQLEHLLLDLQMILNGINNYKNPKLTEMLTAKFYMPKKATEL | 356 |
| | KHLQCLEEELKPLEEVLNLAQSKNFHLRPRDLISRINVIVLELKGSETTFMCEYA | |
| | DETATIVEFLNRWITFAQSIISTLT | |

In some embodiments, the IL-2 polypeptides of the present disclosure also contain other modifications, including but not limited to mutations and deletions, that provide additional advantages such as improved biophysical properties. Improved biophysical properties include but are not limited to improved thermostability, aggregation propensity, acid reversibility, viscosity, and production in a mammalian or bacterial or yeast cell. For example, residue C125 may be replaced with a neutral amino acid such as serine, alanine, threonine or valine; and N terminal A1 residue could be deleted, both of which were described in U.S. Pat. No. 4,518,584. IL-2 polypeptides may also include a mutation of the residue M104, such as M104A, as described in U.S. Pat. No. 5,206,344. Thus, in certain embodiments the IL-2 polypeptide of the present disclosure comprises the amino acid substitution C125A. In other embodiments, one, two, or three N-terminal residues are deleted.

### Exemplary IL-2 Fusion Proteins

In some embodiments, a fusion protein of the present disclosure comprises one, two, or all three polypeptides shown for a single fusion protein in Table 5. In some embodiments, a fusion protein of the present disclosure comprises two light chains, a heavy chain comprising an IL-2 fusion, and a heavy chain not comprising an IL-2 fusion for a single fusion protein in Table 5. As is known in the art, the C-terminal lysine of some antibody heavy chain species may be cleaved off in some fraction of molecules. Therefore, in some embodiments, a fusion protein of the present disclosure comprises two light chains, a heavy chain comprising an IL-2 fusion, and a heavy chain not comprising an IL-2 fusion for a single fusion protein in Table 5, wherein the heavy chain not comprising the IL-2 fusion comprises the sequence of SEQ ID Nos:158, 161, 164, 167, 170, 173, 176, 189, 300, 304, 308, 312, 326, 320, 324, 328, 332, 336, 340, 344, 348, or 352 for the respective fusion protein. In some embodiments, a fusion protein of the present disclosure comprises two light chains, a heavy chain comprising an IL-2 fusion, and a heavy chain not comprising an IL-2 fusion for a single fusion protein in Table 5, wherein the heavy chain not comprising the IL-2 fusion comprises the sequence of SEQ ID Nos:217-224, 301, 305, 309, 313, 317, 321, 325, 329, 333, 337, 341, 345, 349, or 353 for the respective fusion protein. In some embodiments, the present disclosure provides a plurality of fusion proteins of the present disclosure (e.g., in a mixture), wherein each fusion protein of the plurality comprises two light chains, a heavy chain comprising an IL-2 fusion, and a heavy chain not comprising an IL-2 fusion for a single fusion protein in Table 5, wherein the heavy chain not comprising the IL-2 fusion comprises the sequence of SEQ ID Nos:158, 161, 164, 167, 170, 173, 176, 189, 300, 304, 308, 312, 326, 320, 324, 328, 332, 336, 340, 344, 348, or 352 for the respective fusion protein, or the sequence of SEQ ID Nos:217-224, 301, 305, 309, 313, 317, 321, 325, 329, 333, 337, 341, 345, 349, or 353 for the respective fusion protein, or the plurality comprises a mixture of species representing cleaved (e.g., comprising the sequence of SEQ ID Nos:158, 161, 164, 167, 170, 173, 176, 189, 300, 304, 308, 312, 326, 320, 324, 328, 332, 336, 340, 344, 348, or 352 for the respective fusion protein) and uncleaved (e.g., comprising the sequence of SEQ ID Nos: 217-224, 301, 305, 309, 313, 317, 321, 325, 329, 333, 337, 341, 345, 349, or 353 for the respective fusion protein) species.

For example, in some embodiments, a fusion protein of the present disclosure comprises one or two light chains comprising the amino acid sequence of SEQ ID NO: 156, a heavy chain comprising the amino acid sequence of SEQ ID NO: 157, and a heavy chain comprising the amino acid sequence of SEQ ID NO: 158. In some embodiments, a fusion protein of the present disclosure comprises one or two light chains comprising the amino acid sequence of SEQ ID NO: 159, a heavy chain comprising the amino acid sequence of SEQ ID NO: 160, and a heavy chain comprising the amino acid sequence of SEQ ID NO: 161. In some embodiments, a fusion protein of the present disclosure comprises one or two light chains comprising the amino acid sequence of SEQ ID NO: 162, a heavy chain comprising the amino acid sequence of SEQ ID NO: 163, and a heavy chain comprising the amino acid sequence of SEQ ID NO: 164. In some embodiments, a fusion protein of the present disclosure comprises one or two light chains comprising the amino acid sequence of SEQ ID NO: 165, a heavy chain comprising the amino acid sequence of SEQ ID NO: 166, and a heavy chain comprising the amino acid sequence of SEQ ID NO: 167. In some embodiments, a fusion protein of the present disclosure comprises one or two light chains comprising the amino acid sequence of SEQ ID NO: 168, a heavy chain comprising the amino acid sequence of SEQ ID NO: 169, and a heavy chain comprising the amino acid sequence of SEQ ID NO: 170. In some embodiments, a fusion protein of the present disclosure comprises one or two light chains comprising the amino acid sequence of SEQ ID NO: 171, a heavy chain comprising the amino acid sequence of SEQ ID NO: 172, and a heavy chain comprising the amino acid sequence of SEQ ID NO: 173. In some embodiments, a fusion protein of the present disclosure comprises one or two light chains comprising the amino acid sequence of SEQ ID NO: 174, a heavy chain comprising the amino acid sequence of SEQ ID NO: 175, and a heavy chain comprising the amino acid sequence of SEQ ID NO: 176. In some embodiments, a fusion protein of the present disclosure comprises one or two light chains comprising the amino acid sequence of SEQ ID NO: 187, a heavy chain comprising the amino acid sequence of SEQ ID NO: 188, and a heavy chain comprising the amino acid sequence of SEQ ID NO: 189. In some embodiments, a fusion protein of the present disclosure comprises one or two light chains comprising the amino acid sequence of SEQ ID NO: 298, a heavy chain comprising the amino acid sequence of SEQ ID NO: 299, and a heavy chain comprising the amino acid sequence of SEQ ID NO: 300. In some embodiments, a fusion protein of the present disclosure comprises one or two light chains comprising the amino acid sequence of SEQ ID NO: 302, a heavy chain comprising the amino acid sequence of SEQ ID NO: 303, and a heavy chain comprising the amino acid sequence of SEQ ID NO: 304. In some embodiments, a fusion protein of the present disclosure comprises one or two light chains comprising the amino acid sequence of SEQ ID NO: 306, a heavy chain comprising the amino acid sequence of SEQ ID NO: 307, and a heavy chain comprising the amino acid sequence of SEQ ID NO: 308. In some embodiments, a fusion protein of the present disclosure comprises one or two light chains comprising the amino acid sequence of SEQ ID NO: 310, a heavy chain comprising the amino acid sequence of SEQ ID NO: 311, and a heavy chain comprising the amino acid sequence of SEQ ID NO: 312. In some embodiments, a fusion protein of the present disclosure comprises one or two light chains comprising the amino acid sequence of SEQ ID NO: 314, a heavy chain comprising the amino acid sequence of SEQ ID NO: 315, and a heavy chain comprising the amino acid sequence of SEQ ID NO: 316. In some embodiments, a fusion protein of the present disclosure comprises one or two light chains comprising the amino acid sequence of SEQ ID NO: 318, a heavy chain comprising the amino acid sequence of SEQ ID NO: 319, and a heavy chain comprising the amino acid sequence of SEQ ID NO: 320. In some embodiments, a fusion protein of the present disclosure comprises one or two light chains comprising the amino acid sequence of SEQ ID NO: 322, a heavy chain comprising the amino acid sequence of SEQ ID NO: 323, and a heavy chain comprising the amino acid sequence of SEQ ID NO: 324. In some embodiments, a fusion protein of the present disclosure comprises one or two light chains comprising the amino acid sequence of SEQ ID NO: 326, a heavy chain comprising the amino acid sequence of SEQ ID NO: 327, and a heavy chain comprising the amino acid sequence of SEQ ID NO: 328. In some embodiments, a fusion protein of the present disclosure comprises one or two light chains comprising the amino acid sequence of SEQ ID NO: 334, a heavy chain comprising the amino acid sequence of SEQ ID NO: 335, and a heavy chain comprising the amino acid sequence of SEQ ID NO: 336. In some embodiments, a fusion protein of the present disclosure comprises one or two light chains comprising the amino acid sequence of SEQ ID NO: 338, a heavy chain comprising the amino acid sequence of SEQ ID NO: 339, and a heavy chain comprising the amino acid sequence of SEQ ID NO: 340. In some embodiments, a fusion protein of the present disclosure comprises one or two light chains comprising the amino acid sequence of SEQ ID NO: 342, a heavy chain comprising the amino acid sequence of SEQ ID NO: 343, and a heavy chain comprising the amino acid sequence of SEQ ID NO: 344. In some embodiments, a fusion protein of the present disclosure comprises one or two light chains comprising the amino acid sequence of SEQ ID NO: 346, a heavy chain comprising the amino acid sequence of SEQ ID NO: 347, and a heavy chain comprising the amino acid sequence of SEQ ID NO: 348. In some embodiments, a fusion protein of the present disclosure comprises one or two light chains comprising the amino acid sequence of SEQ ID NO: 350, a heavy chain comprising the amino acid sequence of SEQ ID NO: 351, and a heavy chain comprising the amino acid sequence of SEQ ID NO: 352.

In some embodiments, a fusion protein of the present disclosure comprises one or two light chains comprising the amino acid sequence of SEQ ID NO: 156, a heavy chain comprising the amino acid sequence of SEQ ID NO: 157, and a heavy chain comprising the amino acid sequence of SEQ ID NO: 217. In some embodiments, a fusion protein of the present disclosure comprises one or two light chains comprising the amino acid sequence of SEQ ID NO: 159, a heavy chain comprising the amino acid sequence of SEQ ID NO: 160, and a heavy chain comprising the amino acid sequence of SEQ ID NO: 218. In some embodiments, a fusion protein of the present disclosure comprises one or two light chains comprising the amino acid sequence of SEQ ID NO: 162, a heavy chain comprising the amino acid sequence of SEQ ID NO: 163, and a heavy chain comprising the amino acid sequence of SEQ ID NO: 219. In some embodiments, a fusion protein of the present disclosure comprises one or two light chains comprising the amino acid sequence of SEQ ID NO: 165, a heavy chain comprising the amino acid sequence of SEQ ID NO: 166, and a heavy chain comprising the amino acid sequence of SEQ ID NO: 220. In some embodiments, a fusion protein of the present disclosure comprises one or two light chains comprising the amino acid sequence of SEQ ID NO: 168, a heavy chain comprising the amino acid sequence of SEQ ID NO: 169, and a heavy chain comprising the amino acid sequence of SEQ ID NO: 221. In some embodiments, a fusion protein of the present disclosure comprises one or two light chains comprising the amino acid sequence of SEQ ID NO: 171, a heavy chain comprising the amino acid sequence of SEQ ID NO: 172, and a heavy chain comprising the amino acid sequence of SEQ ID NO: 222. In some embodiments, a fusion protein of the present disclosure comprises one or two light chains comprising the amino acid sequence of SEQ ID NO: 174, a heavy chain comprising the amino acid sequence of SEQ ID NO: 175, and a heavy chain comprising the amino acid sequence of SEQ ID NO: 223. In some embodiments, a fusion protein of the present disclosure comprises one or two light chains comprising the amino acid sequence of SEQ ID NO: 187, a heavy chain comprising the amino acid sequence of SEQ ID NO: 188, and a heavy chain comprising the amino acid sequence of SEQ ID NO: 224. In some embodiments, a fusion protein of the present disclosure comprises one or two light chains comprising the amino acid sequence of SEQ ID NO: 298, a heavy chain comprising the amino acid sequence of SEQ ID NO: 299, and a heavy chain comprising the amino acid sequence of SEQ ID NO: 301. In some embodiments, a fusion protein of the present disclosure comprises one or two light chains comprising the amino acid sequence of SEQ ID NO: 302, a heavy chain comprising the amino acid sequence of SEQ ID NO: 303, and a heavy chain comprising the amino acid sequence of SEQ ID NO: 305. In some embodiments, a fusion protein of the present disclosure comprises one or two light chains comprising the amino acid sequence of SEQ ID NO: 306, a heavy chain comprising the amino acid sequence of SEQ ID NO: 307, and a heavy chain comprising the amino acid sequence of SEQ ID NO: 309. In some embodiments, a fusion protein of the present disclosure comprises one or two light chains comprising the amino acid sequence of SEQ ID NO: 310, a heavy chain comprising the amino acid sequence of SEQ ID NO: 311, and a heavy chain comprising the amino acid sequence of SEQ ID NO: 313. In some embodiments, a fusion protein of the present disclosure comprises one or two light chains comprising the amino acid sequence of SEQ ID NO: 314, a heavy chain comprising the amino acid sequence of SEQ ID NO: 315, and a heavy chain comprising the amino acid sequence of SEQ ID NO: 317. In some embodiments, a fusion protein of the present disclosure comprises one or two light chains comprising the amino acid sequence of SEQ ID NO: 318, a heavy chain comprising the amino acid sequence of SEQ ID NO: 319, and a heavy chain comprising the amino acid sequence of SEQ ID NO: 321. In some embodiments, a fusion protein of the present disclosure comprises one or two light chains comprising the amino acid sequence of SEQ ID NO: 322, a heavy chain comprising the amino acid sequence of SEQ ID NO: 323, and a heavy chain comprising the amino acid sequence of SEQ ID NO: 325. In some embodiments, a fusion protein of the present disclosure comprises one or two light chains comprising the amino acid sequence of SEQ ID NO: 326, a heavy chain comprising the amino acid sequence of SEQ ID NO: 327, and a heavy chain comprising the amino acid sequence of SEQ ID NO: 329. In some embodiments, a fusion protein of the present disclosure comprises one or two light chains comprising the amino acid sequence of SEQ ID NO: 330, a heavy chain comprising the amino acid sequence of SEQ ID NO: 331, and a heavy chain comprising the amino acid sequence of SEQ ID NO: 333. In some embodiments, a fusion protein of the present disclosure comprises one or two light chains comprising the amino acid sequence of SEQ ID NO: 334, a heavy chain comprising the amino acid sequence of SEQ ID NO: 335, and a heavy chain comprising the amino acid sequence of SEQ ID NO: 337. In some embodiments, a fusion protein of the present disclosure comprises one or two light chains comprising the amino acid sequence of SEQ ID NO: 338, a heavy chain comprising the amino acid sequence of SEQ ID NO: 339, and a heavy chain comprising the amino acid sequence of SEQ ID NO: 341. In some embodiments, a fusion protein of the present disclosure comprises one or two light chains comprising the amino acid sequence of SEQ ID NO: 342, a heavy chain comprising the amino acid sequence of SEQ ID NO: 343, and a heavy chain comprising the amino acid sequence of SEQ ID NO: 345. In some embodiments, a fusion protein of the present disclosure comprises one or two light chains comprising the amino acid sequence of SEQ ID NO: 346, a heavy chain comprising the amino acid sequence of SEQ ID NO: 347, and a heavy chain comprising the amino acid sequence of SEQ ID NO: 349. In some embodiments, a fusion protein of the present disclosure comprises one or two light chains comprising the amino acid sequence of SEQ ID NO: 350, a heavy chain comprising the amino acid sequence of SEQ ID NO: 351, and a heavy chain comprising the amino acid sequence of SEQ ID NO: 353.

In some embodiments, the present disclosure provides a mixture of fusion protein species, wherein each species comprises one or two light chains comprising the amino acid sequence of SEQ ID NO: 156, a heavy chain comprising the amino acid sequence of SEQ ID NO: 157, and a heavy chain comprising the amino acid sequence of SEQ ID NO: 158 or 217. In some embodiments, the present disclosure provides a mixture of fusion protein species, wherein each species comprises one or two light chains comprising the amino acid sequence of SEQ ID NO: 159, a heavy chain comprising the amino acid sequence of SEQ ID NO: 160, and a heavy chain comprising the amino acid sequence of SEQ ID NO: 161 or 218. In some embodiments, the present disclosure provides a mixture of fusion protein species, wherein each species comprises one or two light chains comprising the amino acid sequence of SEQ ID NO: 162, a heavy chain comprising the amino acid sequence of SEQ ID NO: 163, and a heavy chain comprising the amino acid sequence of SEQ ID NO: 164 or 219. In some embodiments, the present disclosure provides a mixture of fusion protein species, wherein each species comprises one or two light chains comprising the amino acid sequence of SEQ ID NO: 165, a heavy chain comprising the amino acid sequence of SEQ ID NO: 166, and a heavy chain comprising the amino acid sequence of SEQ ID NO: 167 or 220. In some embodiments, the present disclosure provides a mixture of fusion protein species, wherein each species comprises one or two light chains comprising the amino acid sequence of SEQ ID NO: 168, a heavy chain comprising the amino acid sequence of SEQ ID NO: 169, and a heavy chain comprising the amino acid sequence of SEQ ID NO: 170 or 221. In some embodiments, the present disclosure provides a mixture of fusion protein species, wherein each species comprises one or two light chains comprising the amino acid sequence of SEQ ID NO: 171, a heavy chain comprising the amino acid sequence of SEQ ID NO: 172, and a heavy chain comprising the amino acid sequence of SEQ ID NO: 173 or 222. In some embodiments, the present disclosure provides a mixture of fusion protein species, wherein each species comprises one or two light chains comprising the amino acid sequence of SEQ ID NO: 174, a heavy chain comprising the amino acid sequence of SEQ ID NO: 175, and a heavy chain comprising the amino acid sequence of SEQ ID NO: 176 or 223. In some embodiments, the present disclosure provides a mixture of fusion protein species, wherein each species comprises one or two light chains comprising the amino acid sequence of SEQ ID NO: 187, a heavy chain comprising the amino acid sequence of SEQ ID NO: 188, and a heavy chain comprising the amino acid sequence of SEQ ID NO: 189 or 224. In some embodiments, the present disclosure provides a mixture of fusion protein species, wherein each species comprises one or two light chains comprising the amino acid sequence of SEQ ID NO: 298, a heavy chain comprising the amino acid sequence of SEQ ID NO: 299, and a heavy chain comprising the amino acid sequence of SEQ ID NO: 300 or 301. In some embodiments, the present disclosure provides a mixture of fusion protein species, wherein each species comprises one or two light chains comprising the amino acid sequence of SEQ ID NO: 302, a heavy chain comprising the amino acid sequence of SEQ ID NO: 303, and a heavy chain comprising the amino acid sequence of SEQ ID NO: 304 or 305. In some embodiments, the present disclosure provides a mixture of fusion protein species, wherein each species comprises one or two light chains comprising the amino acid sequence of SEQ ID NO: 306, a heavy chain comprising the amino acid sequence of SEQ ID NO: 307, and a heavy chain comprising the amino acid sequence of SEQ ID NO: 308 or 309. In some embodiments, the present disclosure provides a mixture of fusion protein species, wherein each species comprises one or two light chains comprising the amino acid sequence of SEQ ID NO: 310, a heavy chain comprising the amino acid sequence of SEQ ID NO: 311, and a heavy chain comprising the amino acid sequence of SEQ ID NO: 312 or 313. In some embodiments, the present disclosure provides a mixture of fusion protein species, wherein each species comprises one or two light chains comprising the amino acid sequence of SEQ ID NO: 314, a heavy chain comprising the amino acid sequence of SEQ ID NO: 315, and a heavy chain comprising the amino acid sequence of SEQ ID NO: 316 or 317. In some embodiments, the present disclosure provides a mixture of fusion protein species, wherein each species comprises one or two light chains comprising the amino acid sequence of SEQ ID NO: 318, a heavy chain comprising the amino acid sequence of SEQ ID NO: 319, and a heavy chain comprising the amino acid sequence of SEQ ID NO: 320 or 321. In some embodiments, the present disclosure provides a mixture of fusion protein species, wherein each species comprises one or two light chains comprising the amino acid sequence of SEQ ID NO: 322, a heavy chain comprising the amino acid sequence of SEQ ID NO: 323, and a heavy chain comprising the amino acid sequence of SEQ ID NO: 324 or 325. In some embodiments, the present disclosure provides a mixture of fusion protein species, wherein each species comprises one or two light chains comprising the amino acid sequence of SEQ ID NO: 326, a heavy chain comprising the amino acid sequence of SEQ ID NO: 327, and a heavy chain comprising the amino acid sequence of SEQ ID NO: 328 or 329. In some embodiments, the present disclosure provides a mixture of fusion protein species, wherein each species comprises one or two light chains comprising the amino acid sequence of SEQ ID NO: 330, a heavy chain comprising the amino acid sequence of SEQ ID NO: 331, and a heavy chain comprising the amino acid sequence of SEQ ID NO: 332 or 333. In some embodiments, the present disclosure provides a mixture of fusion protein species, wherein each species comprises one or two light chains comprising the amino acid sequence of SEQ ID NO: 334, a heavy chain comprising the amino acid sequence of SEQ ID NO: 335, and a heavy chain comprising the amino acid sequence of SEQ ID NO: 336 or 337. In some embodiments, the present disclosure provides a mixture of fusion protein species, wherein each species comprises one or two light chains comprising the amino acid sequence of SEQ ID NO: 338, a heavy chain comprising the amino acid sequence of SEQ ID NO: 339, and a heavy chain comprising the amino acid sequence of SEQ ID NO: 340 or 341. In some embodiments, the present disclosure provides a mixture of fusion protein species, wherein each species comprises one or two light chains comprising the amino acid sequence of SEQ ID NO: 342, a heavy chain comprising the amino acid sequence of SEQ ID NO: 343, and a heavy chain comprising the amino acid sequence of SEQ ID NO: 344 or 345. In some embodiments, the present disclosure provides a mixture of fusion protein species, wherein each species comprises one or two light chains comprising the amino acid sequence of SEQ ID NO: 346, a heavy chain comprising the amino acid sequence of SEQ ID NO: 347, and a heavy chain comprising the amino acid sequence of SEQ ID NO: 348 or 349. In some embodiments, the present disclosure provides a mixture of fusion protein species, wherein each species comprises one or two light chains comprising the amino acid sequence of SEQ ID NO: 350, a heavy chain comprising the amino acid sequence of SEQ ID NO: 351, and a heavy chain comprising the amino acid sequence of SEQ ID NO: 352 or 353.

In some embodiments, the present disclosure provides a fusion protein comprising two heavy chain sequences and two light chain sequences of a single fusion protein listed in Table 5, wherein one of the heavy chain sequences has an IL2 fusion and the other heavy chain sequence is without an IL2 fusion, and wherein the two light chain sequences are identical. In some embodiments, the heavy chain sequence without an IL2 fusion comprises a lysine at the C terminus. In some embodiments, the fusion protein is of format A shown in FIG. 7. For example, in some embodiments, the fusion protein comprises four polypeptide chains, wherein (1) the first polypeptide chain comprises the amino acid sequence of SEQ ID NO: 334, the second polypeptide chain comprises the amino acid sequence of SEQ ID NO: 335, the third polypeptide chain comprises the amino acid sequence of SEQ ID NO: 336, and the fourth polypeptide chain comprises the amino acid sequence of SEQ ID NO: 334; (2) the first polypeptide chain comprises the amino acid sequence of SEQ ID NO: 334, the second polypeptide chain comprises the amino acid sequence of SEQ ID NO: 335, the third polypeptide chain comprises the amino acid sequence of SEQ ID NO: 337, and the fourth polypeptide chain comprises the amino acid sequence of SEQ ID NO: 334; (3) the first polypeptide chain comprises the amino acid sequence of SEQ ID NO: 338, the second polypeptide chain comprises the amino acid sequence of SEQ ID NO: 339, the third polypeptide chain comprises the amino acid sequence of SEQ ID NO: 340, and the fourth polypeptide chain comprises the amino acid sequence of SEQ ID NO: 338; or (4) the first polypeptide chain comprises the amino acid sequence of SEQ ID NO: 338, the second polypeptide chain comprises the amino acid sequence of SEQ ID NO: 339, the third polypeptide chain comprises the amino acid sequence of SEQ ID NO: 341, and the fourth polypeptide chain comprises the amino acid sequence of SEQ ID NO: 338.

TABLE 5
Anti-CD8: IL2 fusion protein sequences

| | | | | Heavy chain |
|---|---|---|---|---|
| | | | | sequence |
| | | Heavy chain | Heavy chain | (without IL2 |
| | Light chain | sequence | sequence (without | fusion) plus C- |
| Name | sequence | (with IL2 fusion) | IL2 fusion) | term lysine |
| xhCD8v1- | DIQMTQSPASLS | QVHLQQSGPELVKPG | QVHLQQSGPELV | QVHLQQSGPE |
| IL-2m1 | ASVGETVTITC | ASVKMSCKTSGYTFT | KPGASVKMSCK | LVKPGASVKM |
| | GASENIYGALN | KYTMHWVKQGHEES | TSGYTFTKYTM | SCKTSGYTFTK |
| | WYQRKQGKSP | LEWIGHFNPNNDETK | HWVKQGHEESL | YTMHWVKQG |
| | QLLIFGATNLA | YNQKFTGKATLTVD | EWIGHFNPNNDE | HEESLEWIGHF |
| | DGVSSRFSGSG | KSSTTAYMELRSLTS | TKYNQKFTGKA | NPNNDETKYN |
| | SDRQYSLKISSL | DDSALYYCARDGLG | TLTVDKSSTTAY | QKFTGKATLT |
| | HPDDVATYYC | LRLFADWGQGTLVT | MELRSLTSDDSA | VDKSSTTAYM |
| | QNILDTPWTFG | VSSASTKGPSVFPLAP | LYYCARDGLGL | ELRSLTSDDSA |
| | GGTKLEIKRTV | SSKSTSGGTAALGCL | RLFADWGQGTL | LYYCARDGLG |
| | AAPSVFIFPPSD | VKDYFPEPVTVSWNS | VTVSSASTKGPS | LRLFADWGQG |
| | EQLKSGTASVV | GALTSGVHTFPAVLQ | VFPLAPSSKSTSG | TLVTVSSASTK |
| | CLLNNFYPREA | SSGLYSLSSVVTVPSS | GTAALGCLVKD | GPSVFPLAPSS |
| | KVQWKVDNAL | SLGTQTYICNVNHKP | YFPEPVTVSWNS | KSTSGGTAAL |
| | QSGNSQESVTE | SNTKVDKKVEPKSCD | GALTSGVHTFPA | GCLVKDYFPEP |
| | QDSKDSTYSLS | KTHTCPPCPAPEAAG | VLQSSGLYSLSS | VTVSWNSGAL |
| | STLTLSKADYE | APSVFLFPPKPKDTL | VVTVPSSSLGTQ | TSGVHTFPAVL |
| | KHKVYACEVT | MISRTPEVTCVVVDV | TYICNVNHKPSN | QSSGLYSLSSV |
| | HQGLSSPVTKS | SHEDPEVKFNWYVD | TKVDKKVEPKS | VTVPSSSLGTQ |
| | FNRGEC | GVEVHNAKTKPREE | CDKTHTCPPCPA | TYICNVNHKPS |
| | (SEQ ID NO: | QYNSTYRVVSVLTVL | PEAAGAPSVFLF | NTKVDKKVEP |
| | 156) | HQDWLNGKEYKCKV | PPKPKDTLMISR | KSCDKTHTCPP |
| | | SNKALPAPIEKTISKA | TPEVTCVVVDVS | CPAPEAAGAPS |
| | | KGQPREPQVYTLPPC | HEDPEVKFNWY | VFLFPPKPKDT |
| | | REEMTKNQVSLSCA | VDGVEVHNAKT | LMISRTPEVTC |
| | | VKGFYPSDIAVEWES | KPREEQYNSTYR | VVVDVSHEDP |
| | | NGQPENNYKTTPPVL | VVSVLTVLHQD | EVKFNWYVDG |
| | | DSDGSFFLVSKLTVD | WLNGKEYKCKV | VEVHNAKTKP |
| | | KSRWQQGNVFSCSV | SNKALPAPIEKTI | REEQYNSTYR |
| | | MHEALHNHYTQKSL | SKAKGQPREPQV | VVSVLTVLHQ |
| | | SLSPGSGGGGSGGGG | CTLPPSREEMTK | DWLNGKEYKC |
| | | SGGGGSAPTSSSTKK | NQVSLWCLVKG | KVSNKALPAPI |
| | | TQLQLEHLLLDLQMI | FYPSDIAVEWES | EKTISKAKGQP |
| | | LNGINNYKNPKLTEM | NGQPENNYKTTP | REPQVCTLPPS |
| | | LTAKFYMPKKATEL | PVLDSDGSFFLY | REEMTKNQVS |
| | | KHLQCLEEELKPLEE | SKLTVDKSRWQ | LWCLVKGFYP |
| | | VLNLAQSKNFHLRPR | QGNVFSCSVMH | SDIAVEWESNG |
| | | DLISAINVIVLELKGS | EALHNHYTQKS | QPENNYKTTPP |
| | | ETTFMCEYADETATI | LSLSPG | VLDSDGSFFLY |
| | | VEFLNRWITFAQSIIS | (SEQ ID NO: 158) | SKLTVDKSRW |
| | | TLT | | QQGNVFSCSV |
| | | (SEQ ID NO: 157) | | MHEALHNHYT |
| | | | | QKSLSLSPGK |
| | | | | (SEQ ID NO: |
| | | | | 217) |
| xhCD8v2- | DIQMTQSPSSLS | QVQLVQSGAEVKKP | QVQLVQSGAEV | QVQLVQSGAE |
| IL-2m1 | ASVGDRVTITC | GSSVKVSCKASGYRF | KKPGSSVKVSCK | VKKPGSSVKV |
| | RASQEIYGALN | HNFAISWVRQAPGQ | ASGYRFHNFAIS | SCKASGYRFH |
| | WYQQKPGKAP | GLEWMGGIIPGHAKA | WVRQAPGQGLE | NFAISWVRQAP |
| | KLLIYGATNLQ | NYAQKFQGRVTITAD | WMGGIIPGHAK | GQGLEWMGGI |
| | SGVPSRFSGSGS | ESTSTAYMELSSLRSE | ANYAQKFQGRV | IPGHAKANYA |
| | GTDFTLTISSLQ | DTAVYYCARDGLGIR | TITADESTSTAY | QKFQGRVTITA |
| | PEDFATYYCQD | LFADWGQGTLVTVS | MELSSLRSEDTA | DESTSTAYMEL |
| | IYDAPWTFGGG | SASTKGPSVFPLAPSS | VYYCARDGLGIR | SSLRSEDTAVY |
| | TKVEIKRTVAA | KSTSGGTAALGCLVK | LFADWGQGTLV | YCARDGLGIRL |
| | PSVFIFPPSDEQ | DYFPEPVTVSWNSGA | TVSSASTKGPSV | FADWGQGTLV |
| | LKSGTASVVCL | LTSGVHTFPAVLQSS | FPLAPSSKSTSGG | TVSSASTKGPS |
| | LNNFYPREAKV | GLYSLSSVVTVPSSSL | TAALGCLVKDY | VFPLAPSSKST |
| | QWKVDNALQS | GTQTYICNVNHKPSN | FPEPVTVSWNSG | SGGTAALGCL |
| | GNSQESVTEQD | TKVDKKVEPKSCDK | ALTSGVHTFPAV | VKDYFPEPVTV |
| | SKDSTYSLSSTL | THTCPPCPAPEAAGA | LQSSGLYSLSSV | SWNSGALTSG |
| | TLSKADYEKHK | PSVFLFPPKPKDTLMI | VTVPSSSLGTQT | VHTFPAVLQSS |
| | VYACEVTHQGL | SRTPEVTCVVVDVSH | YICNVNHKPSNT | GLYSLSSVVTV |
| | SSPVTKSFNRGE | EDPEVKFNWYVDGV | KVDKKVEPKSC | PSSSLGTQTYIC |
| | C | EVHNAKTKPREEQY | DKTHTCPPCPAP | NVNHKPSNTK |
| | (SEQ ID NO: | NSTYRVVSVLTVLHQ | EAAGAPSVFLFP | VDKKVEPKSC |
| | 159) | DWLNGKEYKCKVSN | PKPKDTLMISRT | DKTHTCPPCPA |
| | | KALPAPIEKTISKAKG | PEVTCVVVDVS | PEAAGAPSVFL |
| | | QPREPQVYTLPPCRE | HEDPEVKFNWY | FPPKPKDTLMI |
| | | EMTKNQVSLSCAVK | VDGVEVHNAKT | SRTPEVTCVVV |
| | | GFYPSDIAVEWESNG | KPREEQYNSTYR | DVSHEDPEVKF |
| | | QPENNYKTTPPVLDS | VVSVLTVLHQD | NWYVDGVEV |
| | | DGSFFLVSKLTVDKS | WLNGKEYKCKV | HNAKTKPREE |
| | | RWQQGNVFSCSVMH | SNKALPAPIEKTI | QYNSTYRVVS |
| | | EALHNHYTQKSLSLS | SKAKGQPREPQV | VLTVLHQDWL |
| | | PGSGGGGSGGGGSG | CTLPPSREEMTK | NGKEYKCKVS |
| | | GGGSAPTSSSTKKTQ | NQVSLWCLVKG | NKALPAPIEKTI |
| | | LQLEHLLLDLQMILN | FYPSDIAVEWES | SKAKGQPREP |
| | | GINNYKNPKLTEMLT | NGQPENNYKTTP | QVCTLPPSREE |
| | | AKFYMPKKATELKH | PVLDSDGSFFLY | MTKNQVSLWC |
| | | LQCLEEELKPLEEVL | SKLTVDKSRWQ | LVKGFYPSDIA |
| | | NLAQSKNFHLRPRDL | QGNVFSCSVMH | VEWESNGQPE |
| | | ISAINVIVLELKGSET | EALHNHYTQKS | NNYKTTPPVL |
| | | TFMCEYADETATIVE | LSLSPG | DSDGSFFLYSK |
| | | FLNRWITFAQSIISTLT | (SEQ ID NO: 161) | LTVDKSRWQQ |
| | | (SEQ ID NO: 160) | | GNVFSCSVMH |
| | | | | EALHNHYTQK |
| | | | | SLSLSPGK |
| | | | | (SEQ ID NO: |
| | | | | 218) |
| xhCD8v3- | DIQMTQSPSSLS | QVQLVQSGAEVKKP | QVQLVQSGAEV | QVQLVQSGAE |
| IL-2m1 | ASVGDRVTITC | GSSVKVSCKASGSRF | KKPGSSVKVSCK | VKKPGSSVKV |
| | RASQEIYGALN | YKFAISWVRQAPGQ | ASGSRFYKFAIS | SCKASGSRFYK |
| | WYQQKPGKAP | GLEWMGGIIPGHAKA | WVRQAPGQGLE | FAISWVRQAPG |
| | KLLIYGATNLQ | NYAQKFQGRVTITAD | WMGGIIPGHAK | QGLEWMGGIIP |
| | SGVPSRFSGSGS | ESTSTAYMELSSLRSE | ANYAQKFQGRV | GHAKANYAQK |
| | GTDFTLTISSLQ | DTAVYYCARDGLGIR | TITADESTSTAY | FQGRVTITADE |
| | PEDFATYYCQD | LFADWGQGTLVTVS | MELSSLRSEDTA | STSTAYMELSS |
| | IYDAPWTFGGG | SASTKGPSVFPLAPSS | VYYCARDGLGIR | LRSEDTAVYY |
| | TKVEIKRTVAA | KSTSGGTAALGCLVK | LFADWGQGTLV | CARDGLGIRLF |
| | PSVFIFPPSDEQ | DYFPEPVTVSWNSGA | TVSSASTKGPSV | ADWGQGTLVT |
| | LKSGTASVVCL | LTSGVHTFPAVLQSS | FPLAPSSKSTSGG | VSSASTKGPSV |
| | LNNFYPREAKV | GLYSLSSVVTVPSSSL | TAALGCLVKDY | FPLAPSSKSTS |
| | QWKVDNALQS | GTQTYICNVNHKPSN | FPEPVTVSWNSG | GGTAALGCLV |
| | GNSQESVTEQD | TKVDKKVEPKSCDK | ALTSGVHTFPAV | KDYFPEPVTVS |
| | SKDSTYSLSSTL | THTCPPCPAPEAAGA | LQSSGLYSLSSV | WNSGALTSGV |
| | TLSKADYEKHK | PSVFLFPPKPKDTLMI | VTVPSSSLGTQT | HTFPAVLQSSG |
| | VYACEVTHQGL | SRTPEVTCVVVDVSH | YICNVNHKPSNT | LYSLSSVVTVP |
| | SSPVTKSFNRGE | EDPEVKFNWYVDGV | KVDKKVEPKSC | SSSLGTQTYIC |
| | C | EVHNAKTKPREEQY | DKTHTCPPCPAP | NVNHKPSNTK |
| | (SEQ ID NO: | NSTYRVVSVLTVLHQ | EAAGAPSVFLFP | VDKKVEPKSC |
| | 162) | DWLNGKEYKCKVSN | PKPKDTLMISRT | DKTHTCPPCPA |
| | | KALPAPIEKTISKAKG | PEVTCVVVDVS | PEAAGAPSVFL |
| | | QPREPQVYTLPPCRE | HEDPEVKFNWY | FPPKPKDTLMI |
| | | EMTKNQVSLSCAVK | VDGVEVHNAKT | SRTPEVTCVVV |
| | | GFYPSDIAVEWESNG | KPREEQYNSTYR | DVSHEDPEVKF |
| | | QPENNYKTTPPVLDS | VVSVLTVLHQD | NWYVDGVEV |
| | | DGSFFLVSKLTVDKS | WLNGKEYKCKV | HNAKTKPREE |
| | | RWQQGNVFSCSVMH | SNKALPAPIEKTI | QYNSTYRVVS |
| | | EALHNHYTQKSLSLS | SKAKGQPREPQV | VLTVLHQDWL |
| | | PGSGGGGSGGGGSG | CTLPPSREEMTK | NGKEYKCKVS |
| | | GGGSAPTSSSTKKTQ | NQVSLWCLVKG | NKALPAPIEKTI |
| | | LQLEHLLLDLQMILN | FYPSDIAVEWES | SKAKGQPREP |
| | | GINNYKNPKLTEMLT | NGQPENNYKTTP | QVCTLPPSREE |
| | | AKFYMPKKATELKH | PVLDSDGSFFLY | MTKNQVSLWC |
| | | LQCLEEELKPLEEVL | SKLTVDKSRWQ | LVKGFYPSDIA |
| | | NLAQSKNFHLRPRDL | QGNVFSCSVMH | VEWESNGQPE |
| | | ISAINVIVLELKGSET | EALHNHYTQKS | NNYKTTPPVL |
| | | TFMCEYADETATIVE | LSLSPG | DSDGSFFLYSK |
| | | FLNRWITFAQSIISTLT | (SEQ ID NO: 164) | LTVDKSRWQQ |
| | | (SEQ ID NO: 163) | | GNVFSCSVMH |
| | | | | EALHNHYTQK |
| | | | | SLSLSPGK |
| | | | | (SEQ ID NO: |
| | | | | 219) |
| xhCD8v4- | DIQMTQSPSSLS | QVQLVQSGAEVKKP | QVQLVQSGAEV | QVQLVQSGAE |
| IL-2m1 | ASVGDRVTITC | GSSVKVSCKASGYTF | KKPGSSVKVSCK | VKKPGSSVKV |
| | RASQKIYGALN | TKYAISWVRQAPGQ | ASGYTFTKYAIS | SCKASGYTFTK |
| | WYQQKPGKAP | GLEWMGGIIPGHAKA | WVRQAPGQGLE | YAISWVRQAP |
| | KLLIYGATNLQ | NYAQKFQGRVTITAD | WMGGIIPGHAK | GQGLEWMGGI |
| | SGVPSRFSGSGS | ESTSTAYMELSSLRSE | ANYAQKFQGRV | IPGHAKANYA |
| | GTDFTLTISSLQ | DTAVYYCARDGLGIR | TITADESTSTAY | QKFQGRVTITA |
| | PEDFATYYCQN | LFADWGQGTLVTVS | MELSSLRSEDTA | DESTSTAYMEL |
| | TYDTPWTFGGG | SASTKGPSVFPLAPSS | VYYCARDGLGIR | SSLRSEDTAVY |
| | TKVEIKRTVAA | KSTSGGTAALGCLVK | LFADWGQGTLV | YCARDGLGIRL |
| | PSVFIFPPSDEQ | DYFPEPVTVSWNSGA | TVSSASTKGPSV | FADWGQGTLV |
| | LKSGTASVVCL | LTSGVHTFPAVLQSS | FPLAPSSKSTSGG | TVSSASTKGPS |
| | LNNFYPREAKV | GLYSLSSVVTVPSSSL | TAALGCLVKDY | VFPLAPSSKST |
| | QWKVDNALQS | GTQTYICNVNHKPSN | FPEPVTVSWNSG | SGGTAALGCL |
| | GNSQESVTEQD | TKVDKKVEPKSCDK | ALTSGVHTFPAV | VKDYFPEPVTV |
| | SKDSTYSLSSTL | THTCPPCPAPEAAGA | LQSSGLYSLSSV | SWNSGALTSG |
| | TLSKADYEKHK | PSVFLFPPKPKDTLMI | VTVPSSSLGTQT | VHTFPAVLQSS |
| | VYACEVTHQGL | SRTPEVTCVVVDVSH | YICNVNHKPSNT | GLYSLSSVVTV |
| | SSPVTKSFNRGE | EDPEVKFNWYVDGV | KVDKKVEPKSC | PSSSLGTQTYIC |
| | C | EVHNAKTKPREEQY | DKTHTCPPCPAP | NVNHKPSNTK |
| | (SEQ ID NO: | NSTYRVVSVLTVLHQ | EAAGAPSVFLFP | VDKKVEPKSC |
| | 165) | DWLNGKEYKCKVSN | PKPKDTLMISRT | DKTHTCPPCPA |
| | | KALPAPIEKTISKAKG | PEVTCVVVDVS | PEAAGAPSVFL |
| | | QPREPQVYTLPPCRE | HEDPEVKFNWY | FPPKPKDTLMI |
| | | EMTKNQVSLSCAVK | VDGVEVHNAKT | SRTPEVTCVVV |
| | | GFYPSDIAVEWESNG | KPREEQYNSTYR | DVSHEDPEVKF |
| | | QPENNYKTTPPVLDS | VVSVLTVLHQD | NWYVDGVEV |
| | | DGSFFLVSKLTVDKS | WLNGKEYKCKV | HNAKTKPREE |
| | | RWQQGNVFSCSVMH | SNKALPAPIEKTI | QYNSTYRVVS |
| | | EALHNHYTQKSLSLS | SKAKGQPREPQV | VLTVLHQDWL |
| | | PGSGGGGSGGGGSG | CTLPPSREEMTK | NGKEYKCKVS |
| | | GGGSAPTSSSTKKTQ | NQVSLWCLVKG | NKALPAPIEKTI |
| | | LQLEHLLLDLQMILN | FYPSDIAVEWES | SKAKGQPREP |
| | | GINNYKNPKLTEMLT | NGQPENNYKTTP | QVCTLPPSREE |
| | | AKFYMPKKATELKH | PVLDSDGSFFLY | MTKNQVSLWC |
| | | LQCLEEELKPLEEVL | SKLTVDKSRWQ | LVKGFYPSDIA |
| | | NLAQSKNFHLRPRDL | QGNVFSCSVMH | VEWESNGQPE |
| | | ISAINVIVLELKGSET | EALHNHYTQKS | NNYKTTPPVL |
| | | TFMCEYADETATIVE | LSLSPG | DSDGSFFLYSK |
| | | FLNRWITFAQSIISTLT | (SEQ ID NO: 167) | LTVDKSRWQQ |
| | | (SEQ ID NO: 166) | | GNVFSCSVMH |
| | | | | EALHNHYTQK |
| | | | | SLSLSPGK |
| | | | | (SEQ ID NO: |
| | | | | 220) |
| xhCD8v5- | DIQMTQSPSSLS | QVQLVQSGAEVKKP | QVQLVQSGAEV | QVQLVQSGAE |
| IL-2m1 | ASVGDRVTITC | GSSVKVSCKASGSGF | KKPGSSVKVSCK | VKKPGSSVKV |
| | RASQKIYGALN | RGHAISWVRQAPGQ | ASGSGFRGHAIS | SCKASGSGFRG |
| | WYQQKPGKAP | GLEWMGGIIPGHAKA | WVRQAPGQGLE | HAISWVRQAP |
| | KLLIYGATNLQ | NYAQKFQGRVTITAD | WMGGIIPGHAK | GQGLEWMGGI |
| | SGVPSRFSGSGS | ESTSTAYMELSSLRSE | ANYAQKFQGRV | IPGHAKANYA |
| | GTDFTLTISSLQ | DTAVYYCARDGLGIR | TITADESTSTAY | QKFQGRVTITA |
| | PEDFATYYCQN | LFADWGQGTLVTVS | MELSSLRSEDTA | DESTSTAYMEL |
| | TYDTPWTFGGG | SASTKGPSVFPLAPSS | VYYCARDGLGIR | SSLRSEDTAVY |
| | TKVEIKRTVAA | KSTSGGTAALGCLVK | LFADWGQGTLV | YCARDGLGIRL |
| | PSVFIFPPSDEQ | DYFPEPVTVSWNSGA | TVSSASTKGPSV | FADWGQGTLV |
| | LKSGTASVVCL | LTSGVHTFPAVLQSS | FPLAPSSKSTSGG | TVSSASTKGPS |
| | LNNFYPREAKV | GLYSLSSVVTVPSSSL | TAALGCLVKDY | VFPLAPSSKST |
| | QWKVDNALQS | GTQTYICNVNHKPSN | FPEPVTVSWNSG | SGGTAALGCL |
| | GNSQESVTEQD | TKVDKKVEPKSCDK | ALTSGVHTFPAV | VKDYFPEPVTV |
| | SKDSTYSLSSTL | THTCPPCPAPEAAGA | LQSSGLYSLSSV | SWNSGALTSG |
| | TLSKADYEKHK | PSVFLFPPKPKDTLMI | VTVPSSSLGTQT | VHTFPAVLQSS |
| | VYACEVTHQGL | SRTPEVTCVVVDVSH | YICNVNHKPSNT | GLYSLSSVVTV |
| | SSPVTKSFNRGE | EDPEVKFNWYVDGV | KVDKKVEPKSC | PSSSLGTQTYIC |
| | C | EVHNAKTKPREEQY | DKTHTCPPCPAP | NVNHKPSNTK |
| | (SEQ ID NO: | NSTYRVVSVLTVLHQ | EAAGAPSVFLFP | VDKKVEPKSC |
| | 168) | DWLNGKEYKCKVSN | PKPKDTLMISRT | DKTHTCPPCPA |
| | | KALPAPIEKTISKAKG | PEVTCVVVDVS | PEAAGAPSVFL |
| | | QPREPQVYTLPPCRE | HEDPEVKFNWY | FPPKPKDTLMI |
| | | EMTKNQVSLSCAVK | VDGVEVHNAKT | SRTPEVTCVVV |
| | | GFYPSDIAVEWESNG | KPREEQYNSTYR | DVSHEDPEVKF |
| | | QPENNYKTTPPVLDS | VVSVLTVLHQD | NWYVDGVEV |
| | | DGSFFLVSKLTVDKS | WLNGKEYKCKV | HNAKTKPREE |
| | | RWQQGNVFSCSVMH | SNKALPAPIEKTI | QYNSTYRVVS |
| | | EALHNHYTQKSLSLS | SKAKGQPREPQV | VLTVLHQDWL |
| | | PGSGGGGSGGGGSG | CTLPPSREEMTK | NGKEYKCKVS |
| | | GGGSAPTSSSTKKTQ | NQVSLWCLVKG | NKALPAPIEKTI |
| | | LQLEHLLLDLQMILN | FYPSDIAVEWES | SKAKGQPREP |
| | | GINNYKNPKLTEMLT | NGQPENNYKTTP | QVCTLPPSREE |
| | | AKFYMPKKATELKH | PVLDSDGSFFLY | MTKNQVSLWC |
| | | LQCLEEELKPLEEVL | SKLTVDKSRWQ | LVKGFYPSDIA |
| | | NLAQSKNFHLRPRDL | QGNVFSCSVMH | VEWESNGQPE |
| | | ISAINVIVLELKGSET | EALHNHYTQKS | NNYKTTPPVL |
| | | TFMCEYADETATIVE | LSLSPG | DSDGSFFLYSK |
| | | FLNRWITFAQSIISTLT | (SEQ ID NO: 170) | LTVDKSRWQQ |
| | | (SEQ ID NO: 169) | | GNVFSCSVMH |
| | | | | EALHNHYTQK |
| | | | | SLSLSPGK |
| | | | | (SEQ ID NO: |
| | | | | 221) |
| xhCD8v6- | EIVLTQSPATLS | EVQLVESGGGAVRP | EVQLVESGGGA | EVQLVESGGG |
| IL-2m1 | VSPGERATLSC | GGSLRLSCAASGFTF | VRPGGSLRLSCA | AVRPGGSLRLS |
| | RASQSVSSNLA | DDYGMSWVRQAPG | ASGFTFDDYGM | CAASGFTFDD |
| | WYQQKPGQAP | KGLEWVSDINWSGEI | SWVRQAPGKGL | YGMSWVRQAP |
| | RLLIYGASSRAT | TAYADSVKGRFTISR | EWVSDINWSGEI | GKGLEWVSDI |
| | GIPDRFSGSGSG | DNAKNSLYLQMNSL | TAYADSVKGRF | NWSGEITAYA |
| | TDFTLTISRLEP | RAEDTAVYYCARSN | TISRDNAKNSLY | DSVKGRFTISR |
| | EDFAVYYCQQ | SYRWDDALDIWGQG | LQMNSLRAEDT | DNAKNSLYLQ |
| | YGSSPPVTFGQ | TMVTVSSASTKGPSV | AVYYCARSNSY | MNSLRAEDTA |
| | GTKVEIKRTVA | FPLAPSSKSTSGGTAA | RWDDALDIWGQ | VYYCARSNSY |
| | APSVFIFPPSDE | LGCLVKDYFPEPVTV | GTMVTVSSASTK | RWDDALDIWG |
| | QLKSGTASVVC | SWNSGALTSGVHTFP | GPSVFPLAPSSKS | QGTMVTVSSA |
| | LLNNFYPREAK | AVLQSSGLYSLSSVV | TSGGTAALGCLV | STKGPSVFPLA |
| | VQWKVDNALQ | TVPSSSLGTQTYICNV | KDYFPEPVTVSW | PSSKSTSGGTA |
| | SGNSQESVTEQ | NHKPSNTKVDKKVE | NSGALTSGVHTF | ALGCLVKDYF |
| | DSKDSTYSLSST | PKSCDKTHTCPPCPA | PAVLQSSGLYSL | PEPVTVSWNS |
| | LTLSKADYEKH | PEAAGAPSVFLFPPKP | SSVVTVPSSSLG | GALTSGVHTFP |
| | KVYACEVTHQ | KDTLMISRTPEVTCV | TQTYICNVNHKP | AVLQSSGLYSL |
| | GLSSPVTKSFNR | VVDVSHEDPEVKFN | SNTKVDKKVEP | SSVVTVPSSSL |
| | GEC | WYVDGVEVHNAKT | KSCDKTHTCPPC | GTQTYICNVN |
| | (SEQ ID NO: | KPREEQYNSTYRVVS | PAPEAAGAPSVF | HKPSNTKVDK |
| | 171) | VLTVLHQDWLNGKE | LFPPKPKDTLMIS | KVEPKSCDKT |
| | | YKCKVSNKALPAPIE | RTPEVTCVVVD | HTCPPCPAPEA |
| | | KTISKAKGQPREPQV | VSHEDPEVKFN | AGAPSVFLFPP |
| | | YTLPPCREEMTKNQV | WYVDGVEVHN | KPKDTLMISRT |
| | | SLSCAVKGFYPSDIA | AKTKPREEQYNS | PEVTCVVVDV |
| | | VEWESNGQPENNYK | TYRVVSVLTVLH | SHEDPEVKFN |
| | | TTPPVLDSDGSFFLVS | QDWLNGKEYKC | WYVDGVEVH |
| | | KLTVDKSRWQQGNV | KVSNKALPAPIE | NAKTKPREEQ |
| | | FSCSVMHEALHNHY | KTISKAKGQPRE | YNSTYRVVSV |
| | | TQKSLSLSPGSGGGG | PQVCTLPPSREE | LTVLHQDWLN |
| | | SGGGGSGGGGSAPTS | MTKNQVSLWCL | GKEYKCKVSN |
| | | SSTKKTQLQLEHLLL | VKGFYPSDIAVE | KALPAPIEKTIS |
| | | DLQMILNGINNYKNP | WESNGQPENNY | KAKGQPREPQ |
| | | KLTEMLTAKFYMPK | KTTPPVLDSDGS | VCTLPPSREEM |
| | | KATELKHLQCLEEEL | FFLYSKLTVDKS | TKNQVSLWCL |
| | | KPLEEVLNLAQSKNF | RWQQGNVFSCS | VKGFYPSDIAV |
| | | HLRPRDLISAINVIVL | VMHEALHNHYT | EWESNGQPEN |
| | | ELKGSETTFMCEYAD | QKSLSLSPG | NYKTTPPVLDS |
| | | ETATIVEFLNRWITFA | (SEQ ID NO: 173) | DGSFFLYSKLT |
| | | QSIISTLT | | VDKSRWQQGN |
| | | (SEQ ID NO: 172) | | VFSCSVMHEA |
| | | | | LHNHYTQKSL |
| | | | | SLSPGK |
| | | | | (SEQ ID NO: |
| | | | | 222) |
| xhCD8v7- | EIVLTQSPATLS | EVQLVESGGGLVQPG | EVQLVESGGGL | EVQLVESGGG |
| IL-2m1 | VTPGEGATLSC | RSLRLSCAASGFTFD | VQPGRSLRLSCA | LVQPGRSLRLS |
| | RASHSVGSNLA | DYAMHWVRQAPGK | ASGFTFDDYAM | CAASGFTFDD |
| | WYQQKPGQAP | GLEWVAVISYDGSN | HWVRQAPGKGL | YAMHWVRQA |
| | RLLIYDASNRA | KYYADSVKGRFTISR | EWVAVISYDGS | PGKGLEWVAV |
| | TGIPARFSGSGS | DNSKNTLYLQMNSL | NKYYADSVKGR | ISYDGSNKYYA |
| | GTDFTLTISSLE | RAEDTAVYYCAKDRI | FTISRDNSKNTL | DSVKGRFTISR |
| | PEDLAVYYCQQ | GWYDYDAFDIWGQG | YLQMNSLRAED | DNSKNTLYLQ |
| | RSNWPPTFGQG | TMVTVSSASTKGPSV | TAVYYCAKDRI | MNSLRAEDTA |
| | TRLEIKRTVAAP | FPLAPSSKSTSGGTAA | GWYDYDAFDIW | VYYCAKDRIG |
| | SVFIFPPSDEQL | LGCLVKDYFPEPVTV | GQGTMVTVSSA | WYDYDAFDIW |
| | KSGTASVVCLL | SWNSGALTSGVHTFP | STKGPSVFPLAPS | GQGTMVTVSS |
| | NNFYPREAKVQ | AVLQSSGLYSLSSVV | SKSTSGGTAALG | ASTKGPSVFPL |
| | WKVDNALQSG | TVPSSSLGTQTYICNV | CLVKDYFPEPVT | APSSKSTSGGT |
| | NSQESVTEQDS | NHKPSNTKVDKKVE | VSWNSGALTSG | AALGCLVKDY |
| | KDSTYSLSSTLT | PKSCDKTHTCPPCPA | VHTFPAVLQSSG | FPEPVTVSWNS |
| | LSKADYEKHKV | PEAAGAPSVFLFPPKP | LYSLSSVVTVPS | GALTSGVHTFP |
| | YACEVTHQGLS | KDTLMISRTPEVTCV | SSLGTQTYICNV | AVLQSSGLYSL |
| | SPVTKSFNRGE | VVDVSHEDPEVKFN | NHKPSNTKVDK | SSVVTVPSSSL |
| | C | WYVDGVEVHNAKT | KVEPKSCDKTHT | GTQTYICNVN |
| | (SEQ ID NO: | KPREEQYNSTYRVVS | CPPCPAPEAAGA | HKPSNTKVDK |
| | 174) | VLTVLHQDWLNGKE | PSVFLFPPKPKDT | KVEPKSCDKT |
| | | YKCKVSNKALPAPIE | LMISRTPEVTCV | HTCPPCPAPEA |
| | | KTISKAKGQPREPQV | VVDVSHEDPEV | AGAPSVFLFPP |
| | | YTLPPCREEMTKNQV | KFNWYVDGVEV | KPKDTLMISRT |
| | | SLSCAVKGFYPSDIA | HNAKTKPREEQ | PEVTCVVVDV |
| | | VEWESNGQPENNYK | YNSTYRVVSVLT | SHEDPEVKFN |
| | | TTPPVLDSDGSFFLVS | VLHQDWLNGKE | WYVDGVEVH |
| | | KLTVDKSRWQQGNV | YKCKVSNKALP | NAKTKPREEQ |
| | | FSCSVMHEALHNHY | APIEKTISKAKG | YNSTYRVVSV |
| | | TQKSLSLSPGSGGGG | QPREPQVCTLPP | LTVLHQDWLN |
| | | SGGGGSGGGGSAPTS | SREEMTKNQVSL | GKEYKCKVSN |
| | | SSTKKTQLQLEHLLL | WCLVKGFYPSDI | KALPAPIEKTIS |
| | | DLQMILNGINNYKNP | AVEWESNGQPE | KAKGQPREPQ |
| | | KLTEMLTAKFYMPK | NNYKTTPPVLDS | VCTLPPSREEM |
| | | KATELKHLQCLEEEL | DGSFFLYSKLTV | TKNQVSLWCL |
| | | KPLEEVLNLAQSKNF | DKSRWQQGNVF | VKGFYPSDIAV |
| | | HLRPRDLISAINVIVL | SCSVMHEALHN | EWESNGQPEN |
| | | ELKGSETTFMCEYAD | HYTQKSLSLSPG | NYKTTPPVLDS |
| | | ETATIVEFLNRWITFA | (SEQ ID NO: 176) | DGSFFLYSKLT |
| | | QSIISTLT | | VDKSRWQQGN |
| | | (SEQ ID NO: 175) | | VFSCSVMHEA |
| | | | | LHNHYTQKSL |
| | | | | SLSPGK |
| | | | | (SEQ ID NO: |
| | | | | 223) |
| xhCD8v8- | DIQMTQSPASLS | QVQLQQSGAELVRP | QVQLQQSGAEL | QVQLQQSGAE |
| IL-2m1 | VSVGETVTITCR | GSSVKISCKASGYAF | VRPGSSVKISCK | LVRPGSSVKIS |
| | ASENIYSNLAW | SSYWMNWVKQRPG | ASGYAFSSYWM | CKASGYAFSSY |
| | YQQKQGKSPQL | QGLEWIGQIYPGDGD | NWVKQRPGQGL | WMNWVKQRP |
| | LVYAATNLAD | TNYNGKFKGKATLT | EWIGQIYPGDGD | GQGLEWIGQIY |
| | GVPSRFSGSGS | ADKSSSTAYMQLSSL | TNYNGKFKGKA | PGDGDTNYNG |
| | GTQYSLKINSL | TSEDSAVYFCARSGA | TLTADKSSSTAY | KFKGKATLTA |
| | QSEDFGSYYCQ | AFSSYYAMDYWGQG | MQLSSLTSEDSA | DKSSSTAYMQ |
| | HFWGTPWTFG | TSVTVSSASTKGPSVF | VYFCARSGAAFS | LSSLTSEDSAV |
| | GGTKLEIKRTV | PLAPSSKSTSGGTAA | SYYAMDYWGQ | YFCARSGAAFS |
| | AAPSVFIFPPSD | LGCLVKDYFPEPVTV | GTSVTVSSASTK | SYYAMDYWG |
| | EQLKSGTASVV | SWNSGALTSGVHTFP | GPSVFPLAPSSKS | QGTSVTVSSAS |
| | CLLNNFYPREA | AVLQSSGLYSLSSVV | TSGGTAALGCLV | TKGPSVFPLAP |
| | KVQWKVDNAL | TVPSSSLGTQTYICNV | KDYFPEPVTVSW | SSKSTSGGTAA |
| | QSGNSQESVTE | NHKPSNTKVDKKVE | NSGALTSGVHTF | LGCLVKDYFPE |
| | QDSKDSTYSLS | PKSCDKTHTCPPCPA | PAVLQSSGLYSL | PVTVSWNSGA |
| | STLTLSKADYE | PEAAGAPSVFLFPPKP | SSVVTVPSSSLG | LTSGVHTFPAV |
| | KHKVYACEVT | KDTLMISRTPEVTCV | TQTYICNVNHKP | LQSSGLYSLSS |
| | HQGLSSPVTKS | VVDVSHEDPEVKFN | SNTKVDKKVEP | VVTVPSSSLGT |
| | FNRGEC | WYVDGVEVHNAKT | KSCDKTHTCPPC | QTYICNVNHKP |
| | (SEQ ID NO: | KPREEQYNSTYRVVS | PAPEAAGAPSVF | SNTKVDKKVE |
| | 187) | VLTVLHQDWLNGKE | LFPPKPKDTLMIS | PKSCDKTHTCP |
| | | YKCKVSNKALPAPIE | RTPEVTCVVVD | PCPAPEAAGAP |
| | | KTISKAKGQPREPQV | VSHEDPEVKFN | SVFLFPPKPKD |
| | | YTLPPCREEMTKNQV | WYVDGVEVHN | TLMISRTPEVT |
| | | SLSCAVKGFYPSDIA | AKTKPREEQYNS | CVVVDVSHED |
| | | VEWESNGQPENNYK | TYRVVSVLTVLH | PEVKFNWYVD |
| | | TTPPVLDSDGSFFLVS | QDWLNGKEYKC | GVEVHNAKTK |
| | | KLTVDKSRWQQGNV | KVSNKALPAPIE | PREEQYNSTYR |
| | | FSCSVMHEALHNHY | KTISKAKGQPRE | VVSVLTVLHQ |
| | | TQKSLSLSPGSGGGG | PQVCTLPPSREE | DWLNGKEYKC |
| | | SGGGGSGGGGSAPTS | MTKNQVSLWCL | KVSNKALPAPI |
| | | SSTKKTQLQLEHLLL | VKGFYPSDIAVE | EKTISKAKGQP |
| | | DLQMILNGINNYKNP | WESNGQPENNY | REPQVCTLPPS |
| | | KLTEMLTAKFYMPK | KTTPPVLDSDGS | REEMTKNQVS |
| | | KATELKHLQCLEEEL | FFLYSKLTVDKS | LWCLVKGFYP |
| | | KPLEEVLNLAQSKNF | RWQQGNVFSCS | SDIAVEWESNG |
| | | HLRPRDLISAINVIVL | VMHEALHNHYT | QPENNYKTTPP |
| | | ELKGSETTFMCEYAD | QKSLSLSPG | VLDSDGSFFLY |
| | | ETATIVEFLNRWITFA | (SEQ ID NO: 189) | SKLTVDKSRW |
| | | QSIISTLT | | QQGNVFSCSV |
| | | (SEQ ID NO: 188) | | MHEALHNHYT |
| | | | | QKSLSLSPGK |
| | | | | (SEQ ID NO: |
| | | | | 224) |
| xhCD8v9- | DIQMTQSPSSLS | QVQLVQSGAEVKKP | QVQLVQSGAEV | QVQLVQSGAE |
| IL-2m1 | ASVGDRVTITC | GSSVKVSCKASGGTF | KKPGSSVKVSCK | VKKPGSSVKV |
| | RASQEIYGALN | SSYAISWVRQAPGQG | ASGGTFSSYAIS | SCKASGGTFSS |
| | WYQQKPGKAP | LEWMGGIIPGAATAN | WVRQAPGQGLE | YAISWVRQAP |
| | KLLIYGATNLQ | YAQKFQGRVTITADE | WMGGIIPGAATA | GQGLEWMGGI |
| | SGVPSRFSGSGS | STSTAYMELSSLRSE | NYAQKFQGRVTI | IPGAATANYA |
| | GTDFTLTISSLQ | DTAVYYCARDAAGI | TADESTSTAYME | QKFQGRVTITA |
| | PEDFATYYCQS | RLFADWGQGTLVTV | LSSLRSEDTAVY | DESTSTAYMEL |
| | TYDAPWTFGG | SSASTKGPSVFPLAPS | YCARDAAGIRLF | SSLRSEDTAVY |
| | GTKVEIKRTVA | SKSTSGGTAALGCLV | ADWGQGTLVTV | YCARDAAGIR |
| | APSVFIFPPSDE | KDYFPEPVTVSWNSG | SSASTKGPSVFPL | LFADWGQGTL |
| | QLKSGTASVVC | ALTSGVHTFPAVLQS | APSSKSTSGGTA | VTVSSASTKGP |
| | LLNNFYPREAK | SGLYSLSSVVTVPSSS | ALGCLVKDYFPE | SVFPLAPSSKS |
| | VQWKVDNALQ | LGTQTYICNVNHKPS | PVTVSWNSGAL | TSGGTAALGC |
| | SGNSQESVTEQ | NTKVDKKVEPKSCD | TSGVHTFPAVLQ | LVKDYFPEPVT |
| | DSKDSTYSLSST | KTHTCPPCPAPEAAG | SSGLYSLSSVVT | VSWNSGALTS |
| | LTLSKADYEKH | APSVFLFPPKPKDTL | VPSSSLGTQTYIC | GVHTFPAVLQS |
| | KVYACEVTHQ | MISRTPEVTCVVVDV | NVNHKPSNTKV | SGLYSLSSVVT |
| | GLSSPVTKSFNR | SHEDPEVKFNWYVD | DKKVEPKSCDK | VPSSSLGTQTY |
| | GEC | GVEVHNAKTKPREE | THTCPPCPAPEA | ICNVNHKPSNT |
| | (SEQ ID NO: | QYNSTYRVVSVLTVL | AGAPSVFLFPPK | KVDKKVEPKS |
| | 298) | HQDWLNGKEYKCKV | PKDTLMISRTPE | CDKTHTCPPCP |
| | | SNKALPAPIEKTISKA | VTCVVVDVSHE | APEAAGAPSVF |
| | | KGQPREPQVYTLPPC | DPEVKFNWYVD | LFPPKPKDTLM |
| | | REEMTKNQVSLSCA | GVEVHNAKTKP | ISRTPEVTCVV |
| | | VKGFYPSDIAVEWES | REEQYNSTYRV | VDVSHEDPEV |
| | | NGQPENNYKTTPPVL | VSVLTVLHQDW | KFNWYVDGVE |
| | | DSDGSFFLVSKLTVD | LNGKEYKCKVS | VHNAKTKPRE |
| | | KSRWQQGNVFSCSV | NKALPAPIEKTIS | EQYNSTYRVV |
| | | MHEALHNHYTQKSL | KAKGQPREPQV | SVLTVLHQDW |
| | | SLSPGSGGGGSGGGG | CTLPPSREEMTK | LNGKEYKCKV |
| | | SGGGGSAPTSSSTKK | NQVSLWCLVKG | SNKALPAPIEK |
| | | TQLQLEHLLLDLQMI | FYPSDIAVEWES | TISKAKGQPRE |
| | | LNGINNYKNPKLTEM | NGQPENNYKTTP | PQVCTLPPSRE |
| | | LTAKFYMPKKATEL | PVLDSDGSFFLY | EMTKNQVSLW |
| | | KHLQCLEEELKPLEE | SKLTVDKSRWQ | CLVKGFYPSDI |
| | | VLNLAQSKNFHLRPR | QGNVFSCSVMH | AVEWESNGQP |
| | | DLISAINVIVLELKGS | EALHNHYTQKS | ENNYKTTPPVL |
| | | ETTFMCEYADETATI | LSLSPG | DSDGSFFLYSK |
| | | VEFLNRWITFAQSIIS | (SEQ ID NO: 300) | LTVDKSRWQQ |
| | | TLT | | GNVFSCSVMH |
| | | (SEQ ID NO: 299) | | EALHNHYTQK |
| | | | | SLSLSPGK |
| | | | | (SEQ ID NO: |
| | | | | 301) |
| xhCD8v10- | EIVLTQSPGTLS | EVQLVESGGGLVQPG | EVQLVESGGGL | EVQLVESGGG |
| IL-2m1 | LSPGERATLSCR | GSLRLSCAASGFTFSS | VQPGGSLRLSCA | LVQPGGSLRLS |
| | ASQSVSSNLAW | YAMSWVRQAPGKGL | ASGFTFSSYAMS | CAASGFTFSSY |
| | YQQKPGQAPRL | EWVSDITYAGGSTAY | WVRQAPGKGLE | AMSWVRQAPG |
| | LIYGASSRATGI | ADSVKGRFTISRDNA | WVSDITYAGGST | KGLEWVSDIT |
| | PDRFSGSGSGT | KNSLYLQMNSLRAE | AYADSVKGRFTI | YAGGSTAYAD |
| | DFTLTISRLEPE | DTAVYYCARSNAYA | SRDNAKNSLYL | SVKGRFTISRD |
| | DFAVYYCQQY | WDDALDIWGQGTM | QMNSLRAEDTA | NAKNSLYLQM |
| | GSSPPVTFGQG | VTVSSASTKGPSVFPL | VYYCARSNAYA | NSLRAEDTAV |
| | TKVEIKRTVAA | APSSKSTSGGTAALG | WDDALDIWGQG | YYCARSNAYA |
| | PSVFIFPPSDEQ | CLVKDYFPEPVTVSW | TMVTVSSASTKG | WDDALDIWGQ |
| | LKSGTASVVCL | NSGALTSGVHTFPAV | PSVFPLAPSSKST | GTMVTVSSAS |
| | LNNFYPREAKV | LQSSGLYSLSSVVTV | SGGTAALGCLV | TKGPSVFPLAP |
| | QWKVDNALQS | PSSSLGTQTYICNVN | KDYFPEPVTVSW | SSKSTSGGTAA |
| | GNSQESVTEQD | HKPSNTKVDKKVEP | NSGALTSGVHTF | LGCLVKDYFPE |
| | SKDSTYSLSSTL | KSCDKTHTCPPCPAP | PAVLQSSGLYSL | PVTVSWNSGA |
| | TLSKADYEKHK | EAAGAPSVFLFPPKP | SSVVTVPSSSLG | LTSGVHTFPAV |
| | VYACEVTHQGL | KDTLMISRTPEVTCV | TQTYICNVNHKP | LQSSGLYSLSS |
| | SSPVTKSFNRGE | VVDVSHEDPEVKFN | SNTKVDKKVEP | VVTVPSSSLGT |
| | C | WYVDGVEVHNAKT | KSCDKTHTCPPC | QTYICNVNHKP |
| | (SEQ ID NO: | KPREEQYNSTYRVVS | PAPEAAGAPSVF | SNTKVDKKVE |
| | 302) | VLTVLHQDWLNGKE | LFPPKPKDTLMIS | PKSCDKTHTCP |
| | | YKCKVSNKALPAPIE | RTPEVTCVVVD | PCPAPEAAGAP |
| | | KTISKAKGQPREPQV | VSHEDPEVKFN | SVFLFPPKPKD |
| | | YTLPPCREEMTKNQV | WYVDGVEVHN | TLMISRTPEVT |
| | | SLSCAVKGFYPSDIA | AKTKPREEQYNS | CVVVDVSHED |
| | | VEWESNGQPENNYK | TYRVVSVLTVLH | PEVKFNWYVD |
| | | TTPPVLDSDGSFFLVS | QDWLNGKEYKC | GVEVHNAKTK |
| | | KLTVDKSRWQQGNV | KVSNKALPAPIE | PREEQYNSTYR |
| | | FSCSVMHEALHNHY | KTISKAKGQPRE | VVSVLTVLHQ |
| | | TQKSLSLSPGSGGGG | PQVCTLPPSREE | DWLNGKEYKC |
| | | SGGGGSGGGGSAPTS | MTKNQVSLWCL | KVSNKALPAPI |
| | | SSTKKTQLQLEHLLL | VKGFYPSDIAVE | EKTISKAKGQP |
| | | DLQMILNGINNYKNP | WESNGQPENNY | REPQVCTLPPS |
| | | KLTEMLTAKFYMPK | KTTPPVLDSDGS | REEMTKNQVS |
| | | KATELKHLQCLEEEL | FFLYSKLTVDKS | LWCLVKGFYP |
| | | KPLEEVLNLAQSKNF | RWQQGNVFSCS | SDIAVEWESNG |
| | | HLRPRDLISAINVIVL | VMHEALHNHYT | QPENNYKTTPP |
| | | ELKGSETTFMCEYAD | QKSLSLSPG | VLDSDGSFFLY |
| | | ETATIVEFLNRWITFA | (SEQ ID NO: 304) | SKLTVDKSRW |
| | | QSIISTLT | | QQGNVFSCSV |
| | | (SEQ ID NO: 303) | | MHEALHNHYT |
| | | | | QKSLSLSPGK |
| | | | | (SEQ ID NO: |
| | | | | 305) |
| xhCD8v11- | EIVLTQSPGTLS | EVQLVESGGGLVQPG | EVQLVESGGGL | EVQLVESGGG |
| IL-2m1 | LSPGERATLSCR | GSLRLSCAASGFTFSS | VQPGGSLRLSCA | LVQPGGSLRLS |
| | ASQSVSSNLAW | YAMSWVRQAPGKGL | ASGFTFSSYAMS | CAASGFTFSSY |
| | YQQKPGQAPRL | EWVSDITYAGGSTAY | WVRQAPGKGLE | AMSWVRQAPG |
| | LIYGASSRATGI | ADSVKGRFTISRDNA | WVSDITYAGGST | KGLEWVSDIT |
| | PDRFSGSGSGT | KNSLYLQMNSLRAE | AYADSVKGRFTI | YAGGSTAYAD |
| | DFTLTISRLEPE | DTAVYYCARSNAYA | SRDNAKNSLYL | SVKGRFTISRD |
| | DFAVYYCQQY | WDDALDIWGQGTLV | QMNSLRAEDTA | NAKNSLYLQM |
| | GSSPPVTFGQG | TVSSASTKGPSVFPLA | VYYCARSNAYA | NSLRAEDTAV |
| | TKVEIKRTVAA | PSSKSTSGGTAALGC | WDDALDIWGQG | YYCARSNAYA |
| | PSVFIFPPSDEQ | LVKDYFPEPVTVSWN | TLVTVSSASTKG | WDDALDIWGQ |
| | LKSGTASVVCL | SGALTSGVHTFPAVL | PSVFPLAPSSKST | GTLVTVSSAST |
| | LNNFYPREAKV | QSSGLYSLSSVVTVPS | SGGTAALGCLV | KGPSVFPLAPS |
| | QWKVDNALQS | SSLGTQTYICNVNHK | KDYFPEPVTVSW | SKSTSGGTAAL |
| | GNSQESVTEQD | PSNTKVDKKVEPKSC | NSGALTSGVHTF | GCLVKDYFPEP |
| | SKDSTYSLSSTL | DKTHTCPPCPAPEAA | PAVLQSSGLYSL | VTVSWNSGAL |
| | TLSKADYEKHK | GAPSVFLFPPKPKDTL | SSVVTVPSSSLG | TSGVHTFPAVL |
| | VYACEVTHQGL | MISRTPEVTCVVVDV | TQTYICNVNHKP | QSSGLYSLSSV |
| | SSPVTKSFNRGE | SHEDPEVKFNWYVD | SNTKVDKKVEP | VTVPSSSLGTQ |
| | C | GVEVHNAKTKPREE | KSCDKTHTCPPC | TYICNVNHKPS |
| | (SEQ ID NO: | QYNSTYRVVSVLTVL | PAPEAAGAPSVF | NTKVDKKVEP |
| | 306) | HQDWLNGKEYKCKV | LFPPKPKDTLMIS | KSCDKTHTCPP |
| | | SNKALPAPIEKTISKA | RTPEVTCVVVD | CPAPEAAGAPS |
| | | KGQPREPQVYTLPPC | VSHEDPEVKFN | VFLFPPKPKDT |
| | | REEMTKNQVSLSCA | WYVDGVEVHN | LMISRTPEVTC |
| | | VKGFYPSDIAVEWES | AKTKPREEQYNS | VVVDVSHEDP |
| | | NGQPENNYKTTPPVL | TYRVVSVLTVLH | EVKFNWYVDG |
| | | DSDGSFFLVSKLTVD | QDWLNGKEYKC | VEVHNAKTKP |
| | | KSRWQQGNVFSCSV | KVSNKALPAPIE | REEQYNSTYR |
| | | MHEALHNHYTQKSL | KTISKAKGQPRE | VVSVLTVLHQ |
| | | SLSPGSGGGGSGGGG | PQVCTLPPSREE | DWLNGKEYKC |
| | | SGGGGSAPTSSSTKK | MTKNQVSLWCL | KVSNKALPAPI |
| | | TQLQLEHLLLDLQMI | VKGFYPSDIAVE | EKTISKAKGQP |
| | | LNGINNYKNPKLTEM | WESNGQPENNY | REPQVCTLPPS |
| | | LTAKFYMPKKATEL | KTTPPVLDSDGS | REEMTKNQVS |
| | | KHLQCLEEELKPLEE | FFLYSKLTVDKS | LWCLVKGFYP |
| | | VLNLAQSKNFHLRPR | RWQQGNVFSCS | SDIAVEWESNG |
| | | DLISAINVIVLELKGS | VMHEALHNHYT | QPENNYKTTPP |
| | | ETTFMCEYADETATI | QKSLSLSPG | VLDSDGSFFLY |
| | | VEFLNRWITFAQSIIS | (SEQ ID NO: 308) | SKLTVDKSRW |
| | | TLT | | QQGNVFSCSV |
| | | (SEQ ID NO: 307) | | MHEALHNHYT |
| | | | | QKSLSLSPGK |
| | | | | (SEQ ID NO: |
| | | | | 309) |
| xhCD8v12- | DIQMTQSPSSLS | QVQLVQSGAEVKKP | QVQLVQSGAEV | QVQLVQSGAE |
| IL-2m1 | ASVGDRVTITC | GSSVKVSCKASGGTF | KKPGSSVKVSCK | VKKPGSSVKV |
| | RASQSIYGALN | SSYAISWVRQAPGQG | ASGGTFSSYAIS | SCKASGGTFSS |
| | WYQQKPGKAP | LEWMGGIIPGYATAN | WVRQAPGQGLE | YAISWVRQAP |
| | KLLIYGASNLQ | YAQKFQGRVTITADE | WMGGIIPGYATA | GQGLEWMGGI |
| | SGVPSRFSGSGS | STSTAYMELSSLRSE | NYAQKFQGRVTI | IPGYATANYA |
| | GTDFTLTISSLQ | DTAVYYCARDAAGI | TADESTSTAYME | QKFQGRVTITA |
| | PEDFATYYCQS | RLFADWGQGTLVTV | LSSLRSEDTAVY | DESTSTAYMEL |
| | TYTAPWTFGGG | SSASTKGPSVFPLAPS | YCARDAAGIRLF | SSLRSEDTAVY |
| | TKVEIKRTVAA | SKSTSGGTAALGCLV | ADWGQGTLVTV | YCARDAAGIR |
| | PSVFIFPPSDEQ | KDYFPEPVTVSWNSG | SSASTKGPSVFPL | LFADWGQGTL |
| | LKSGTASVVCL | ALTSGVHTFPAVLQS | APSSKSTSGGTA | VTVSSASTKGP |
| | LNNFYPREAKV | SGLYSLSSVVTVPSSS | ALGCLVKDYFPE | SVFPLAPSSKS |
| | QWKVDNALQS | LGTQTYICNVNHKPS | PVTVSWNSGAL | TSGGTAALGC |
| | GNSQESVTEQD | NTKVDKKVEPKSCD | TSGVHTFPAVLQ | LVKDYFPEPVT |
| | SKDSTYSLSSTL | KTHTCPPCPAPEAAG | SSGLYSLSSVVT | VSWNSGALTS |
| | TLSKADYEKHK | APSVFLFPPKPKDTL | VPSSSLGTQTYIC | GVHTFPAVLQS |
| | VYACEVTHQGL | MISRTPEVTCVVVDV | NVNHKPSNTKV | SGLYSLSSVVT |
| | SSPVTKSFNRGE | SHEDPEVKFNWYVD | DKKVEPKSCDK | VPSSSLGTQTY |
| | C | GVEVHNAKTKPREE | THTCPPCPAPEA | ICNVNHKPSNT |
| | (SEQ ID NO: | QYNSTYRVVSVLTVL | AGAPSVFLFPPK | KVDKKVEPKS |
| | 310) | HQDWLNGKEYKCKV | PKDTLMISRTPE | CDKTHTCPPCP |
| | | SNKALPAPIEKTISKA | VTCVVVDVSHE | APEAAGAPSVF |
| | | KGQPREPQVYTLPPC | DPEVKFNWYVD | LFPPKPKDTLM |
| | | REEMTKNQVSLSCA | GVEVHNAKTKP | ISRTPEVTCVV |
| | | VKGFYPSDIAVEWES | REEQYNSTYRV | VDVSHEDPEV |
| | | NGQPENNYKTTPPVL | VSVLTVLHQDW | KFNWYVDGVE |
| | | DSDGSFFLVSKLTVD | LNGKEYKCKVS | VHNAKTKPRE |
| | | KSRWQQGNVFSCSV | NKALPAPIEKTIS | EQYNSTYRVV |
| | | MHEALHNHYTQKSL | KAKGQPREPQV | SVLTVLHQDW |
| | | SLSPGSGGGGSGGGG | CTLPPSREEMTK | LNGKEYKCKV |
| | | SGGGGSAPTSSSTKK | NQVSLWCLVKG | SNKALPAPIEK |
| | | TQLQLEHLLLDLQMI | FYPSDIAVEWES | TISKAKGQPRE |
| | | LNGINNYKNPKLTEM | NGQPENNYKTTP | PQVCTLPPSRE |
| | | LTAKFYMPKKATEL | PVLDSDGSFFLY | EMTKNQVSLW |
| | | KHLQCLEEELKPLEE | SKLTVDKSRWQ | CLVKGFYPSDI |
| | | VLNLAQSKNFHLRPR | QGNVFSCSVMH | AVEWESNGQP |
| | | DLISAINVIVLELKGS | EALHNHYTQKS | ENNYKTTPPVL |
| | | ETTFMCEYADETATI | LSLSPG | DSDGSFFLYSK |
| | | VEFLNRWITFAQSIIS | (SEQ ID NO: 312) | LTVDKSRWQQ |
| | | TLT | | GNVFSCSVMH |
| | | (SEQ ID NO: 311) | | EALHNHYTQK |
| | | | | SLSLSPGK |
| | | | | (SEQ ID NO: |
| | | | | 313) |
| xhCD8v13- | DIQMTQSPSSLS | QVQLVQSGAEVKKP | QVQLVQSGAEV | QVQLVQSGAE |
| IL-2m1 | ASVGDRVTITC | GSSVKVSCKASGGTF | KKPGSSVKVSCK | VKKPGSSVKV |
| | RASQEIYGALN | SSYAISWVRQAPGQG | ASGGTFSSYAIS | SCKASGGTFSS |
| | WYQQKPGKAP | LEWMGGIIPGYATAN | WVRQAPGQGLE | YAISWVRQAP |
| | KLLIYGATNLQ | YAQKFQGRVTITADE | WMGGIIPGYATA | GQGLEWMGGI |
| | SGVPSRFSGSGS | STSTAYMELSSLRSE | NYAQKFQGRVTI | IPGYATANYA |
| | GTDFTLTISSLQ | DTAVYYCARDAAGI | TADESTSTAYME | QKFQGRVTITA |
| | PEDFATYYCQS | RLFADWGQGTLVTV | LSSLRSEDTAVY | DESTSTAYMEL |
| | TYDAPWTFGG | SSASTKGPSVFPLAPS | YCARDAAGIRLF | SSLRSEDTAVY |
| | GTKVEIKRTVA | SKSTSGGTAALGCLV | ADWGQGTLVTV | YCARDAAGIR |
| | APSVFIFPPSDE | KDYFPEPVTVSWNSG | SSASTKGPSVFPL | LFADWGQGTL |
| | QLKSGTASVVC | ALTSGVHTFPAVLQS | APSSKSTSGGTA | VTVSSASTKGP |
| | LLNNFYPREAK | SGLYSLSSVVTVPSSS | ALGCLVKDYFPE | SVFPLAPSSKS |
| | VQWKVDNALQ | LGTQTYICNVNHKPS | PVTVSWNSGAL | TSGGTAALGC |
| | SGNSQESVTEQ | NTKVDKKVEPKSCD | TSGVHTFPAVLQ | LVKDYFPEPVT |
| | DSKDSTYSLSST | KTHTCPPCPAPEAAG | SSGLYSLSSVVT | VSWNSGALTS |
| | LTLSKADYEKH | APSVFLFPPKPKDTL | VPSSSLGTQTYIC | GVHTFPAVLQS |
| | KVYACEVTHQ | MISRTPEVTCVVVDV | NVNHKPSNTKV | SGLYSLSSVVT |
| | GLSSPVTKSFNR | SHEDPEVKFNWYVD | DKKVEPKSCDK | VPSSSLGTQTY |
| | GEC | GVEVHNAKTKPREE | THTCPPCPAPEA | ICNVNHKPSNT |
| | (SEQ ID NO: | QYNSTYRVVSVLTVL | AGAPSVFLFPPK | KVDKKVEPKS |
| | 314) | HQDWLNGKEYKCKV | PKDTLMISRTPE | CDKTHTCPPCP |
| | | SNKALPAPIEKTISKA | VTCVVVDVSHE | APEAAGAPSVF |
| | | KGQPREPQVYTLPPC | DPEVKFNWYVD | LFPPKPKDTLM |
| | | REEMTKNQVSLSCA | GVEVHNAKTKP | ISRTPEVTCVV |
| | | VKGFYPSDIAVEWES | REEQYNSTYRV | VDVSHEDPEV |
| | | NGQPENNYKTTPPVL | VSVLTVLHQDW | KFNWYVDGVE |
| | | DSDGSFFLVSKLTVD | LNGKEYKCKVS | VHNAKTKPRE |
| | | KSRWQQGNVFSCSV | NKALPAPIEKTIS | EQYNSTYRVV |
| | | MHEALHNHYTQKSL | KAKGQPREPQV | SVLTVLHQDW |
| | | SLSPGSGGGGSGGGG | CTLPPSREEMTK | LNGKEYKCKV |
| | | SGGGGSAPTSSSTKK | NQVSLWCLVKG | SNKALPAPIEK |
| | | TQLQLEHLLLDLQMI | FYPSDIAVEWES | TISKAKGQPRE |
| | | LNGINNYKNPKLTEM | NGQPENNYKTTP | PQVCTLPPSRE |
| | | LTAKFYMPKKATEL | PVLDSDGSFFLY | EMTKNQVSLW |
| | | KHLQCLEEELKPLEE | SKLTVDKSRWQ | CLVKGFYPSDI |
| | | VLNLAQSKNFHLRPR | QGNVFSCSVMH | AVEWESNGQP |
| | | DLISAINVIVLELKGS | EALHNHYTQKS | ENNYKTTPPVL |
| | | ETTFMCEYADETATI | LSLSPG | DSDGSFFLYSK |
| | | VEFLNRWITFAQSIIS | (SEQ ID NO: 316) | LTVDKSRWQQ |
| | | TLT | | GNVFSCSVMH |
| | | (SEQ ID NO: 315) | | EALHNHYTQK |
| | | | | SLSLSPGK |
| | | | | (SEQ ID NO: |
| | | | | 317) |
| xhCD8v14- | EIVLTQSPGTLS | EVQLVESGGGLVQPG | EVQLVESGGGL | EVQLVESGGG |
| IL-2m1 | LSPGERATLSCR | GSLRLSCAASGFTFSS | VQPGGSLRLSCA | LVQPGGSLRLS |
| | ASQSVSSNLAW | YAMSWVRQAPGKGL | ASGFTFSSYAMS | CAASGFTFSSY |
| | YQQKPGQAPRL | EWVSDISYAGGSTAY | WVRQAPGKGLE | AMSWVRQAPG |
| | LIYGASSRATGI | ADSVKGRFTISRDNA | WVSDISYAGGST | KGLEWVSDISY |
| | PDRFSGSGSGT | KNSLYLQMNSLRAE | AYADSVKGRFTI | AGGSTAYADS |
| | DFTLTISRLEPE | DTAVYYCARSNAYA | SRDNAKNSLYL | VKGRFTISRDN |
| | DFAVYYCQQY | WDDALDIWGQGTM | QMNSLRAEDTA | AKNSLYLQMN |
| | GSSPPVTFGQG | VTVSSASTKGPSVFPL | VYYCARSNAYA | SLRAEDTAVY |
| | TKVEIKRTVAA | APSSKSTSGGTAALG | WDDALDIWGQG | YCARSNAYAW |
| | PSVFIFPPSDEQ | CLVKDYFPEPVTVSW | TMVTVSSASTKG | DDALDIWGQG |
| | LKSGTASVVCL | NSGALTSGVHTFPAV | PSVFPLAPSSKST | TMVTVSSAST |
| | LNNFYPREAKV | LQSSGLYSLSSVVTV | SGGTAALGCLV | KGPSVFPLAPS |
| | QWKVDNALQS | PSSSLGTQTYICNVN | KDYFPEPVTVSW | SKSTSGGTAAL |
| | GNSQESVTEQD | HKPSNTKVDKKVEP | NSGALTSGVHTF | GCLVKDYFPEP |
| | SKDSTYSLSSTL | KSCDKTHTCPPCPAP | PAVLQSSGLYSL | VTVSWNSGAL |
| | TLSKADYEKHK | EAAGAPSVFLFPPKP | SSVVTVPSSSLG | TSGVHTFPAVL |
| | VYACEVTHQGL | KDTLMISRTPEVTCV | TQTYICNVNHKP | QSSGLYSLSSV |
| | SSPVTKSFNRGE | VVDVSHEDPEVKFN | SNTKVDKKVEP | VTVPSSSLGTQ |
| | C | WYVDGVEVHNAKT | KSCDKTHTCPPC | TYICNVNHKPS |
| | (SEQ ID NO: | KPREEQYNSTYRVVS | PAPEAAGAPSVF | NTKVDKKVEP |
| | 318) | VLTVLHQDWLNGKE | LFPPKPKDTLMIS | KSCDKTHTCPP |
| | | YKCKVSNKALPAPIE | RTPEVTCVVVD | CPAPEAAGAPS |
| | | KTISKAKGQPREPQV | VSHEDPEVKFN | VFLFPPKPKDT |
| | | YTLPPCREEMTKNQV | WYVDGVEVHN | LMISRTPEVTC |
| | | SLSCAVKGFYPSDIA | AKTKPREEQYNS | VVVDVSHEDP |
| | | VEWESNGQPENNYK | TYRVVSVLTVLH | EVKFNWYVDG |
| | | TTPPVLDSDGSFFLVS | QDWLNGKEYKC | VEVHNAKTKP |
| | | KLTVDKSRWQQGNV | KVSNKALPAPIE | REEQYNSTYR |
| | | FSCSVMHEALHNHY | KTISKAKGQPRE | VVSVLTVLHQ |
| | | TQKSLSLSPGSGGGG | PQVCTLPPSREE | DWLNGKEYKC |
| | | SGGGGSGGGGSAPTS | MTKNQVSLWCL | KVSNKALPAPI |
| | | SSTKKTQLQLEHLLL | VKGFYPSDIAVE | EKTISKAKGQP |
| | | DLQMILNGINNYKNP | WESNGQPENNY | REPQVCTLPPS |
| | | KLTEMLTAKFYMPK | KTTPPVLDSDGS | REEMTKNQVS |
| | | KATELKHLQCLEEEL | FFLYSKLTVDKS | LWCLVKGFYP |
| | | KPLEEVLNLAQSKNF | RWQQGNVFSCS | SDIAVEWESNG |
| | | HLRPRDLISAINVIVL | VMHEALHNHYT | QPENNYKTTPP |
| | | ELKGSETTFMCEYAD | QKSLSLSPG | VLDSDGSFFLY |
| | | ETATIVEFLNRWITFA | (SEQ ID NO: 320) | SKLTVDKSRW |
| | | QSIISTLT | | QQGNVFSCSV |
| | | (SEQ ID NO: 319) | | MHEALHNHYT |
| | | | | QKSLSLSPGK |
| | | | | (SEQ ID NO: |
| | | | | 321) |
| xhCD8v15- | EIVLTQSPGTLS | EVQLVESGGGLVQPG | EVQLVESGGGL | EVQLVESGGG |
| IL-2m1 | LSPGERATLSCR | GSLRLSCAASGFTFSS | VQPGGSLRLSCA | LVQPGGSLRLS |
| | ASQSVSSNLAW | YAMSWVRQAPGKGL | ASGFTFSSYAMS | CAASGFTFSSY |
| | YQQKPGQAPRL | EWVSDISYAGGSTAY | WVRQAPGKGLE | AMSWVRQAPG |
| | LIYGASSRATGI | ADSVKGRFTISRDNA | WVSDISYAGGST | KGLEWVSDISY |
| | PDRFSGSGSGT | KNSLYLQMNSLRAE | AYADSVKGRFTI | AGGSTAYADS |
| | DFTLTISRLEPE | DTAVYYCARSNAYA | SRDNAKNSLYL | VKGRFTISRDN |
| | DFAVYYCQQY | WDDALDIWGQGTLV | QMNSLRAEDTA | AKNSLYLQMN |
| | GSSPPVTFGQG | TVSSASTKGPSVFPLA | VYYCARSNAYA | SLRAEDTAVY |
| | TKVEIKRTVAA | PSSKSTSGGTAALGC | WDDALDIWGQG | YCARSNAYAW |
| | PSVFIFPPSDEQ | LVKDYFPEPVTVSWN | TLVTVSSASTKG | DDALDIWGQG |
| | LKSGTASVVCL | SGALTSGVHTFPAVL | PSVFPLAPSSKST | TLVTVSSASTK |
| | LNNFYPREAKV | QSSGLYSLSSVVTVPS | SGGTAALGCLV | GPSVFPLAPSS |
| | QWKVDNALQS | SSLGTQTYICNVNHK | KDYFPEPVTVSW | KSTSGGTAAL |
| | GNSQESVTEQD | PSNTKVDKKVEPKSC | NSGALTSGVHTF | GCLVKDYFPEP |
| | SKDSTYSLSSTL | DKTHTCPPCPAPEAA | PAVLQSSGLYSL | VTVSWNSGAL |
| | TLSKADYEKHK | GAPSVFLFPPKPKDTL | SSVVTVPSSSLG | TSGVHTFPAVL |
| | VYACEVTHQGL | MISRTPEVTCVVVDV | TQTYICNVNHKP | QSSGLYSLSSV |
| | SSPVTKSFNRGE | SHEDPEVKFNWYVD | SNTKVDKKVEP | VTVPSSSLGTQ |
| | C | GVEVHNAKTKPREE | KSCDKTHTCPPC | TYICNVNHKPS |
| | (SEQ ID NO: | QYNSTYRVVSVLTVL | PAPEAAGAPSVF | NTKVDKKVEP |
| | 322) | HQDWLNGKEYKCKV | LFPPKPKDTLMIS | KSCDKTHTCPP |
| | | SNKALPAPIEKTISKA | RTPEVTCVVVD | CPAPEAAGAPS |
| | | KGQPREPQVYTLPPC | VSHEDPEVKFN | VFLFPPKPKDT |
| | | REEMTKNQVSLSCA | WYVDGVEVHN | LMISRTPEVTC |
| | | VKGFYPSDIAVEWES | AKTKPREEQYNS | VVVDVSHEDP |
| | | NGQPENNYKTTPPVL | TYRVVSVLTVLH | EVKFNWYVDG |
| | | DSDGSFFLVSKLTVD | QDWLNGKEYKC | VEVHNAKTKP |
| | | KSRWQQGNVFSCSV | KVSNKALPAPIE | REEQYNSTYR |
| | | MHEALHNHYTQKSL | KTISKAKGQPRE | VVSVLTVLHQ |
| | | SLSPGSGGGGSGGGG | PQVCTLPPSREE | DWLNGKEYKC |
| | | SGGGGSAPTSSSTKK | MTKNQVSLWCL | KVSNKALPAPI |
| | | TQLQLEHLLLDLQMI | VKGFYPSDIAVE | EKTISKAKGQP |
| | | LNGINNYKNPKLTEM | WESNGQPENNY | REPQVCTLPPS |
| | | LTAKFYMPKKATEL | KTTPPVLDSDGS | REEMTKNQVS |
| | | KHLQCLEEELKPLEE | FFLYSKLTVDKS | LWCLVKGFYP |
| | | VLNLAQSKNFHLRPR | RWQQGNVFSCS | SDIAVEWESNG |
| | | DLISAINVIVLELKGS | VMHEALHNHYT | QPENNYKTTPP |
| | | ETTFMCEYADETATI | QKSLSLSPG | VLDSDGSFFLY |
| | | VEFLNRWITFAQSIIS | (SEQ ID NO: 324) | SKLTVDKSRW |
| | | TLT | | QQGNVFSCSV |
| | | (SEQ ID NO: 323) | | MHEALHNHYT |
| | | | | QKSLSLSPGK |
| | | | | (SEQ ID NO: |
| | | | | 325) |
| xhCD8v9- | DIQMTQSPSSLS | QVQLVQSGAEVKKP | QVQLVQSGAEV | QVQLVQSGAE |
| IL-2m4 | ASVGDRVTITC | GSSVKVSCKASGGTF | KKPGSSVKVSCK | VKKPGSSVKV |
| | RASQEIYGALN | SSYAISWVRQAPGQG | ASGGTFSSYAIS | SCKASGGTFSS |
| | WYQQKPGKAP | LEWMGGIIPGAATAN | WVRQAPGQGLE | YAISWVRQAP |
| | KLLIYGATNLQ | YAQKFQGRVTITADE | WMGGIIPGAATA | GQGLEWMGGI |
| | SGVPSRFSGSGS | STSTAYMELSSLRSE | NYAQKFQGRVTI | IPGAATANYA |
| | GTDFTLTISSLQ | DTAVYYCARDAAGI | TADESTSTAYME | QKFQGRVTITA |
| | PEDFATYYCQS | RLFADWGQGTLVTV | LSSLRSEDTAVY | DESTSTAYMEL |
| | TYDAPWTFGG | SSASTKGPSVFPLAPS | YCARDAAGIRLF | SSLRSEDTAVY |
| | GTKVEIKRTVA | SKSTSGGTAALGCLV | ADWGQGTLVTV | YCARDAAGIR |
| | APSVFIFPPSDE | KDYFPEPVTVSWNSG | SSASTKGPSVFPL | LFADWGQGTL |
| | QLKSGTASVVC | ALTSGVHTFPAVLQS | APSSKSTSGGTA | VTVSSASTKGP |
| | LLNNFYPREAK | SGLYSLSSVVTVPSSS | ALGCLVKDYFPE | SVFPLAPSSKS |
| | VQWKVDNALQ | LGTQTYICNVNHKPS | PVTVSWNSGAL | TSGGTAALGC |
| | SGNSQESVTEQ | NTKVDKKVEPKSCD | TSGVHTFPAVLQ | LVKDYFPEPVT |
| | DSKDSTYSLSST | KTHTCPPCPAPEAAG | SSGLYSLSSVVT | VSWNSGALTS |
| | LTLSKADYEKH | APSVFLFPPKPKDTL | VPSSSLGTQTYIC | GVHTFPAVLQS |
| | KVYACEVTHQ | MISRTPEVTCVVVDV | NVNHKPSNTKV | SGLYSLSSVVT |
| | GLSSPVTKSFNR | SHEDPEVKFNWYVD | DKKVEPKSCDK | VPSSSLGTQTY |
| | GEC | GVEVHNAKTKPREE | THTCPPCPAPEA | ICNVNHKPSNT |
| | (SEQ ID NO: | QYNSTYRVVSVLTVL | AGAPSVFLFPPK | KVDKKVEPKS |
| | 326) | HQDWLNGKEYKCKV | PKDTLMISRTPE | CDKTHTCPPCP |
| | | SNKALPAPIEKTISKA | VTCVVVDVSHE | APEAAGAPSVF |
| | | KGQPREPQVYTLPPC | DPEVKFNWYVD | LFPPKPKDTLM |
| | | REEMTKNQVSLSCA | GVEVHNAKTKP | ISRTPEVTCVV |
| | | VKGFYPSDIAVEWES | REEQYNSTYRV | VDVSHEDPEV |
| | | NGQPENNYKTTPPVL | VSVLTVLHQDW | KFNWYVDGVE |
| | | DSDGSFFLVSKLTVD | LNGKEYKCKVS | VHNAKTKPRE |
| | | KSRWQQGNVFSCSV | NKALPAPIEKTIS | EQYNSTYRVV |
| | | MHEALHNHYTQKSL | KAKGQPREPQV | SVLTVLHQDW |
| | | SLSPGSGGGGSGGGG | CTLPPSREEMTK | LNGKEYKCKV |
| | | SGGGGSAPTSSSTKK | NQVSLWCLVKG | SNKALPAPIEK |
| | | TQLQLEELLLDLQMI | FYPSDIAVEWES | TISKAKGQPRE |
| | | LNGINNYKNPKLTEM | NGQPENNYKTTP | PQVCTLPPSRE |
| | | LTAKFYMPKKATEL | PVLDSDGSFFLY | EMTKNQVSLW |
| | | KHLQCLEEELKPLEE | SKLTVDKSRWQ | CLVKGFYPSDI |
| | | VLNLAQSKNFHLRPR | QGNVFSCSVMH | AVEWESNGQP |
| | | DLISNINVIVLELKGS | EALHNHYTQKS | ENNYKTTPPVL |
| | | ETTFMCEYADETATI | LSLSPG | DSDGSFFLYSK |
| | | VEFLNRWITFAQSIIS | (SEQ ID NO: 328) | LTVDKSRWQQ |
| | | TLT | | GNVFSCSVMH |
| | | (SEQ ID NO: 327) | | EALHNHYTQK |
| | | | | SLSLSPGK |
| | | | | (SEQ ID NO: |
| | | | | 329) |
| xhCD8v10- | EIVLTQSPGTLS | EVQLVESGGGLVQPG | EVQLVESGGGL | EVQLVESGGG |
| IL-2m4 | LSPGERATLSCR | GSLRLSCAASGFTFSS | VQPGGSLRLSCA | LVQPGGSLRLS |
| | ASQSVSSNLAW | YAMSWVRQAPGKGL | ASGFTFSSYAMS | CAASGFTFSSY |
| | YQQKPGQAPRL | EWVSDITYAGGSTAY | WVRQAPGKGLE | AMSWVRQAPG |
| | LIYGASSRATGI | ADSVKGRFTISRDNA | WVSDITYAGGST | KGLEWVSDIT |
| | PDRFSGSGSGT | KNSLYLQMNSLRAE | AYADSVKGRFTI | YAGGSTAYAD |
| | DFTLTISRLEPE | DTAVYYCARSNAYA | SRDNAKNSLYL | SVKGRFTISRD |
| | DFAVYYCQQY | WDDALDIWGQGTM | QMNSLRAEDTA | NAKNSLYLQM |
| | GSSPPVTFGQG | VTVSSASTKGPSVFPL | VYYCARSNAYA | NSLRAEDTAV |
| | TKVEIKRTVAA | APSSKSTSGGTAALG | WDDALDIWGQG | YYCARSNAYA |
| | PSVFIFPPSDEQ | CLVKDYFPEPVTVSW | TMVTVSSASTKG | WDDALDIWGQ |
| | LKSGTASVVCL | NSGALTSGVHTFPAV | PSVFPLAPSSKST | GTMVTVSSAS |
| | LNNFYPREAKV | LQSSGLYSLSSVVTV | SGGTAALGCLV | TKGPSVFPLAP |
| | QWKVDNALQS | PSSSLGTQTYICNVN | KDYFPEPVTVSW | SSKSTSGGTAA |
| | GNSQESVTEQD | HKPSNTKVDKKVEP | NSGALTSGVHTF | LGCLVKDYFPE |
| | SKDSTYSLSSTL | KSCDKTHTCPPCPAP | PAVLQSSGLYSL | PVTVSWNSGA |
| | TLSKADYEKHK | EAAGAPSVFLFPPKP | SSVVTVPSSSLG | LTSGVHTFPAV |
| | VYACEVTHQGL | KDTLMISRTPEVTCV | TQTYICNVNHKP | LQSSGLYSLSS |
| | SSPVTKSFNRGE | VVDVSHEDPEVKFN | SNTKVDKKVEP | VVTVPSSSLGT |
| | C | WYVDGVEVHNAKT | KSCDKTHTCPPC | QTYICNVNHKP |
| | (SEQ ID NO: | KPREEQYNSTYRVVS | PAPEAAGAPSVF | SNTKVDKKVE |
| | 330) | VLTVLHQDWLNGKE | LFPPKPKDTLMIS | PKSCDKTHTCP |
| | | YKCKVSNKALPAPIE | RTPEVTCVVVD | PCPAPEAAGAP |
| | | KTISKAKGQPREPQV | VSHEDPEVKFN | SVFLFPPKPKD |
| | | YTLPPCREEMTKNQV | WYVDGVEVHN | TLMISRTPEVT |
| | | SLSCAVKGFYPSDIA | AKTKPREEQYNS | CVVVDVSHED |
| | | VEWESNGQPENNYK | TYRVVSVLTVLH | PEVKFNWYVD |
| | | TTPPVLDSDGSFFLVS | QDWLNGKEYKC | GVEVHNAKTK |
| | | KLTVDKSRWQQGNV | KVSNKALPAPIE | PREEQYNSTYR |
| | | FSCSVMHEALHNHY | KTISKAKGQPRE | VVSVLTVLHQ |
| | | TQKSLSLSPGSGGGG | PQVCTLPPSREE | DWLNGKEYKC |
| | | SGGGGSGGGGSAPTS | MTKNQVSLWCL | KVSNKALPAPI |
| | | SSTKKTQLQLEELLL | VKGFYPSDIAVE | EKTISKAKGQP |
| | | DLQMILNGINNYKNP | WESNGQPENNY | REPQVCTLPPS |
| | | KLTEMLTAKFYMPK | KTTPPVLDSDGS | REEMTKNQVS |
| | | KATELKHLQCLEEEL | FFLYSKLTVDKS | LWCLVKGFYP |
| | | KPLEEVLNLAQSKNF | RWQQGNVFSCS | SDIAVEWESNG |
| | | HLRPRDLISNINVIVL | VMHEALHNHYT | QPENNYKTTPP |
| | | ELKGSETTFMCEYAD | QKSLSLSPG | VLDSDGSFFLY |
| | | ETATIVEFLNRWITFA | (SEQ ID NO: 332) | SKLTVDKSRW |
| | | QSIISTLT | | QQGNVFSCSV |
| | | (SEQ ID NO: 331) | | MHEALHNHYT |
| | | | | QKSLSLSPGK |
| | | | | (SEQ ID NO: |
| | | | | 333) |
| xhCD8v11- | EIVLTQSPGTLS | EVQLVESGGGLVQPG | EVQLVESGGGL | EVQLVESGGG |
| IL-2m4 | LSPGERATLSCR | GSLRLSCAASGFTFSS | VQPGGSLRLSCA | LVQPGGSLRLS |
| | ASQSVSSNLAW | YAMSWVRQAPGKGL | ASGFTFSSYAMS | CAASGFTFSSY |
| | YQQKPGQAPRL | EWVSDITYAGGSTAY | WVRQAPGKGLE | AMSWVRQAPG |
| | LIYGASSRATGI | ADSVKGRFTISRDNA | WVSDITYAGGST | KGLEWVSDIT |
| | PDRFSGSGSGT | KNSLYLQMNSLRAE | AYADSVKGRFTI | YAGGSTAYAD |
| | DFTLTISRLEPE | DTAVYYCARSNAYA | SRDNAKNSLYL | SVKGRFTISRD |
| | DFAVYYCQQY | WDDALDIWGQGTLV | QMNSLRAEDTA | NAKNSLYLQM |
| | GSSPPVTFGQG | TVSSASTKGPSVFPLA | VYYCARSNAYA | NSLRAEDTAV |
| | TKVEIKRTVAA | PSSKSTSGGTAALGC | WDDALDIWGQG | YYCARSNAYA |
| | PSVFIFPPSDEQ | LVKDYFPEPVTVSWN | TLVTVSSASTKG | WDDALDIWGQ |
| | LKSGTASVVCL | SGALTSGVHTFPAVL | PSVFPLAPSSKST | GTLVTVSSAST |
| | LNNFYPREAKV | QSSGLYSLSSVVTVPS | SGGTAALGCLV | KGPSVFPLAPS |
| | QWKVDNALQS | SSLGTQTYICNVNHK | KDYFPEPVTVSW | SKSTSGGTAAL |
| | GNSQESVTEQD | PSNTKVDKKVEPKSC | NSGALTSGVHTF | GCLVKDYFPEP |
| | SKDSTYSLSSTL | DKTHTCPPCPAPEAA | PAVLQSSGLYSL | VTVSWNSGAL |
| | TLSKADYEKHK | GAPSVFLFPPKPKDTL | SSVVTVPSSSLG | TSGVHTFPAVL |
| | VYACEVTHQGL | MISRTPEVTCVVVDV | TQTYICNVNHKP | QSSGLYSLSSV |
| | SSPVTKSFNRGE | SHEDPEVKFNWYVD | SNTKVDKKVEP | VTVPSSSLGTQ |
| | C | GVEVHNAKTKPREE | KSCDKTHTCPPC | TYICNVNHKPS |
| | (SEQ ID NO: | QYNSTYRVVSVLTVL | PAPEAAGAPSVF | NTKVDKKVEP |
| | 334) | HQDWLNGKEYKCKV | LFPPKPKDTLMIS | KSCDKTHTCPP |
| | | SNKALPAPIEKTISKA | RTPEVTCVVVD | CPAPEAAGAPS |
| | | KGQPREPQVYTLPPC | VSHEDPEVKFN | VFLFPPKPKDT |
| | | REEMTKNQVSLSCA | WYVDGVEVHN | LMISRTPEVTC |
| | | VKGFYPSDIAVEWES | AKTKPREEQYNS | VVVDVSHEDP |
| | | NGQPENNYKTTPPVL | TYRVVSVLTVLH | EVKFNWYVDG |
| | | DSDGSFFLVSKLTVD | QDWLNGKEYKC | VEVHNAKTKP |
| | | KSRWQQGNVFSCSV | KVSNKALPAPIE | REEQYNSTYR |
| | | MHEALHNHYTQKSL | KTISKAKGQPRE | VVSVLTVLHQ |
| | | SLSPGSGGGGSGGGG | PQVCTLPPSREE | DWLNGKEYKC |
| | | SGGGGSAPTSSSTKK | MTKNQVSLWCL | KVSNKALPAPI |
| | | TQLQLEELLLDLQMI | VKGFYPSDIAVE | EKTISKAKGQP |
| | | LNGINNYKNPKLTEM | WESNGQPENNY | REPQVCTLPPS |
| | | LTAKFYMPKKATEL | KTTPPVLDSDGS | REEMTKNQVS |
| | | KHLQCLEEELKPLEE | FFLYSKLTVDKS | LWCLVKGFYP |
| | | VLNLAQSKNFHLRPR | RWQQGNVFSCS | SDIAVEWESNG |
| | | DLISNINVIVLELKGS | VMHEALHNHYT | QPENNYKTTPP |
| | | ETTFMCEYADETATI | QKSLSLSPG | VLDSDGSFFLY |
| | | VEFLNRWITFAQSIIS | (SEQ ID NO: 336) | SKLTVDKSRW |
| | | TLT | | QQGNVFSCSV |
| | | (SEQ ID NO: 335) | | MHEALHNHYT |
| | | | | QKSLSLSPGK |
| | | | | (SEQ ID NO: |
| | | | | 337) |
| xhCD8v12- | DIQMTQSPSSLS | QVQLVQSGAEVKKP | QVQLVQSGAEV | QVQLVQSGAE |
| IL-2m4 | ASVGDRVTITC | GSSVKVSCKASGGTF | KKPGSSVKVSCK | VKKPGSSVKV |
| | RASQSIYGALN | SSYAISWVRQAPGQG | ASGGTFSSYAIS | SCKASGGTFSS |
| | WYQQKPGKAP | LEWMGGIIPGYATAN | WVRQAPGQGLE | YAISWVRQAP |
| | KLLIYGASNLQ | YAQKFQGRVTITADE | WMGGIIPGYATA | GQGLEWMGGI |
| | SGVPSRFSGSGS | STSTAYMELSSLRSE | NYAQKFQGRVTI | IPGYATANYA |
| | GTDFTLTISSLQ | DTAVYYCARDAAGI | TADESTSTAYME | QKFQGRVTITA |
| | PEDFATYYCQS | RLFADWGQGTLVTV | LSSLRSEDTAVY | DESTSTAYMEL |
| | TYTAPWTFGGG | SSASTKGPSVFPLAPS | YCARDAAGIRLF | SSLRSEDTAVY |
| | TKVEIKRTVAA | SKSTSGGTAALGCLV | ADWGQGTLVTV | YCARDAAGIR |
| | PSVFIFPPSDEQ | KDYFPEPVTVSWNSG | SSASTKGPSVFPL | LFADWGQGTL |
| | LKSGTASVVCL | ALTSGVHTFPAVLQS | APSSKSTSGGTA | VTVSSASTKGP |
| | LNNFYPREAKV | SGLYSLSSVVTVPSSS | ALGCLVKDYFPE | SVFPLAPSSKS |
| | QWKVDNALQS | LGTQTYICNVNHKPS | PVTVSWNSGAL | TSGGTAALGC |
| | GNSQESVTEQD | NTKVDKKVEPKSCD | TSGVHTFPAVLQ | LVKDYFPEPVT |
| | SKDSTYSLSSTL | KTHTCPPCPAPEAAG | SSGLYSLSSVVT | VSWNSGALTS |
| | TLSKADYEKHK | APSVFLFPPKPKDTL | VPSSSLGTQTYIC | GVHTFPAVLQS |
| | VYACEVTHQGL | MISRTPEVTCVVVDV | NVNHKPSNTKV | SGLYSLSSVVT |
| | SSPVTKSFNRGE | SHEDPEVKFNWYVD | DKKVEPKSCDK | VPSSSLGTQTY |
| | C | GVEVHNAKTKPREE | THTCPPCPAPEA | ICNVNHKPSNT |
| | (SEQ ID NO: | QYNSTYRVVSVLTVL | AGAPSVFLFPPK | KVDKKVEPKS |
| | 338) | HQDWLNGKEYKCKV | PKDTLMISRTPE | CDKTHTCPPCP |
| | | SNKALPAPIEKTISKA | VTCVVVDVSHE | APEAAGAPSVF |
| | | KGQPREPQVYTLPPC | DPEVKFNWYVD | LFPPKPKDTLM |
| | | REEMTKNQVSLSCA | GVEVHNAKTKP | ISRTPEVTCVV |
| | | VKGFYPSDIAVEWES | REEQYNSTYRV | VDVSHEDPEV |
| | | NGQPENNYKTTPPVL | VSVLTVLHQDW | KFNWYVDGVE |
| | | DSDGSFFLVSKLTVD | LNGKEYKCKVS | VHNAKTKPRE |
| | | KSRWQQGNVFSCSV | NKALPAPIEKTIS | EQYNSTYRVV |
| | | MHEALHNHYTQKSL | KAKGQPREPQV | SVLTVLHQDW |
| | | SLSPGSGGGGSGGGG | CTLPPSREEMTK | LNGKEYKCKV |
| | | SGGGGSAPTSSSTKK | NQVSLWCLVKG | SNKALPAPIEK |
| | | TQLQLEELLLDLQMI | FYPSDIAVEWES | TISKAKGQPRE |
| | | LNGINNYKNPKLTEM | NGQPENNYKTTP | PQVCTLPPSRE |
| | | LTAKFYMPKKATEL | PVLDSDGSFFLY | EMTKNQVSLW |
| | | KHLQCLEEELKPLEE | SKLTVDKSRWQ | CLVKGFYPSDI |
| | | VLNLAQSKNFHLRPR | QGNVFSCSVMH | AVEWESNGQP |
| | | DLISNINVIVLELKGS | EALHNHYTQKS | ENNYKTTPPVL |
| | | ETTFMCEYADETATI | LSLSPG | DSDGSFFLYSK |
| | | VEFLNRWITFAQSIIS | (SEQ ID NO: 340) | LTVDKSRWQQ |
| | | TLT | | GNVFSCSVMH |
| | | (SEQ ID NO: 339) | | EALHNHYTQK |
| | | | | SLSLSPGK |
| | | | | (SEQ ID NO: |
| | | | | 341) |
| xhCD8v13- | DIQMTQSPSSLS | QVQLVQSGAEVKKP | QVQLVQSGAEV | QVQLVQSGAE |
| IL-2m4 | ASVGDRVTITC | GSSVKVSCKASGGTF | KKPGSSVKVSCK | VKKPGSSVKV |
| | RASQEIYGALN | SSYAISWVRQAPGQG | ASGGTFSSYAIS | SCKASGGTFSS |
| | WYQQKPGKAP | LEWMGGIIPGYATAN | WVRQAPGQGLE | YAISWVRQAP |
| | KLLIYGATNLQ | YAQKFQGRVTITADE | WMGGIIPGYATA | GQGLEWMGGI |
| | SGVPSRFSGSGS | STSTAYMELSSLRSE | NYAQKFQGRVTI | IPGYATANYA |
| | GTDFTLTISSLQ | DTAVYYCARDAAGI | TADESTSTAYME | QKFQGRVTITA |
| | PEDFATYYCQS | RLFADWGQGTLVTV | LSSLRSEDTAVY | DESTSTAYMEL |
| | TYDAPWTFGG | SSASTKGPSVFPLAPS | YCARDAAGIRLF | SSLRSEDTAVY |
| | GTKVEIKRTVA | SKSTSGGTAALGCLV | ADWGQGTLVTV | YCARDAAGIR |
| | APSVFIFPPSDE | KDYFPEPVTVSWNSG | SSASTKGPSVFPL | LFADWGQGTL |
| | QLKSGTASVVC | ALTSGVHTFPAVLQS | APSSKSTSGGTA | VTVSSASTKGP |
| | LLNNFYPREAK | SGLYSLSSVVTVPSSS | ALGCLVKDYFPE | SVFPLAPSSKS |
| | VQWKVDNALQ | LGTQTYICNVNHKPS | PVTVSWNSGAL | TSGGTAALGC |
| | SGNSQESVTEQ | NTKVDKKVEPKSCD | TSGVHTFPAVLQ | LVKDYFPEPVT |
| | DSKDSTYSLSST | KTHTCPPCPAPEAAG | SSGLYSLSSVVT | VSWNSGALTS |
| | LTLSKADYEKH | APSVFLFPPKPKDTL | VPSSSLGTQTYIC | GVHTFPAVLQS |
| | KVYACEVTHQ | MISRTPEVTCVVVDV | NVNHKPSNTKV | SGLYSLSSVVT |
| | GLSSPVTKSFNR | SHEDPEVKFNWYVD | DKKVEPKSCDK | VPSSSLGTQTY |
| | GEC | GVEVHNAKTKPREE | THTCPPCPAPEA | ICNVNHKPSNT |
| | (SEQ ID NO: | QYNSTYRVVSVLTVL | AGAPSVFLFPPK | KVDKKVEPKS |
| | 342) | HQDWLNGKEYKCKV | PKDTLMISRTPE | CDKTHTCPPCP |
| | | SNKALPAPIEKTISKA | VTCVVVDVSHE | APEAAGAPSVF |
| | | KGQPREPQVYTLPPC | DPEVKFNWYVD | LFPPKPKDTLM |
| | | REEMTKNQVSLSCA | GVEVHNAKTKP | ISRTPEVTCVV |
| | | VKGFYPSDIAVEWES | REEQYNSTYRV | VDVSHEDPEV |
| | | NGQPENNYKTTPPVL | VSVLTVLHQDW | KFNWYVDGVE |
| | | DSDGSFFLVSKLTVD | LNGKEYKCKVS | VHNAKTKPRE |
| | | KSRWQQGNVFSCSV | NKALPAPIEKTIS | EQYNSTYRVV |
| | | MHEALHNHYTQKSL | KAKGQPREPQV | SVLTVLHQDW |
| | | SLSPGSGGGGSGGGG | CTLPPSREEMTK | LNGKEYKCKV |
| | | SGGGGSAPTSSSTKK | NQVSLWCLVKG | SNKALPAPIEK |
| | | TQLQLEELLLDLQMI | FYPSDIAVEWES | TISKAKGQPRE |
| | | LNGINNYKNPKLTEM | NGQPENNYKTTP | PQVCTLPPSRE |
| | | LTAKFYMPKKATEL | PVLDSDGSFFLY | EMTKNQVSLW |
| | | KHLQCLEEELKPLEE | SKLTVDKSRWQ | CLVKGFYPSDI |
| | | VLNLAQSKNFHLRPR | QGNVFSCSVMH | AVEWESNGQP |
| | | DLISNINVIVLELKGS | EALHNHYTQKS | ENNYKTTPPVL |
| | | ETTFMCEYADETATI | LSLSPG | DSDGSFFLYSK |
| | | VEFLNRWITFAQSIIS | (SEQ ID NO: 344) | LTVDKSRWQQ |
| | | TLT | | GNVFSCSVMH |
| | | (SEQ ID NO: 343) | | EALHNHYTQK |
| | | | | SLSLSPGK |
| | | | | (SEQ ID NO: |
| | | | | 345) |
| xhCD8v14- | EIVLTQSPGTLS | EVQLVESGGGLVQPG | EVQLVESGGGL | EVQLVESGGG |
| IL-2m4 | LSPGERATLSCR | GSLRLSCAASGFTFSS | VQPGGSLRLSCA | LVQPGGSLRLS |
| | ASQSVSSNLAW | YAMSWVRQAPGKGL | ASGFTFSSYAMS | CAASGFTFSSY |
| | YQQKPGQAPRL | EWVSDISYAGGSTAY | WVRQAPGKGLE | AMSWVRQAPG |
| | LIYGASSRATGI | ADSVKGRFTISRDNA | WVSDISYAGGST | KGLEWVSDISY |
| | PDRFSGSGSGT | KNSLYLQMNSLRAE | AYADSVKGRFTI | AGGSTAYADS |
| | DFTLTISRLEPE | DTAVYYCARSNAYA | SRDNAKNSLYL | VKGRFTISRDN |
| | DFAVYYCQQY | WDDALDIWGQGTM | QMNSLRAEDTA | AKNSLYLQMN |
| | GSSPPVTFGQG | VTVSSASTKGPSVFPL | VYYCARSNAYA | SLRAEDTAVY |
| | TKVEIKRTVAA | APSSKSTSGGTAALG | WDDALDIWGQG | YCARSNAYAW |
| | PSVFIFPPSDEQ | CLVKDYFPEPVTVSW | TMVTVSSASTKG | DDALDIWGQG |
| | LKSGTASVVCL | NSGALTSGVHTFPAV | PSVFPLAPSSKST | TMVTVSSAST |
| | LNNFYPREAKV | LQSSGLYSLSSVVTV | SGGTAALGCLV | KGPSVFPLAPS |
| | QWKVDNALQS | PSSSLGTQTYICNVN | KDYFPEPVTVSW | SKSTSGGTAAL |
| | GNSQESVTEQD | HKPSNTKVDKKVEP | NSGALTSGVHTF | GCLVKDYFPEP |
| | SKDSTYSLSSTL | KSCDKTHTCPPCPAP | PAVLQSSGLYSL | VTVSWNSGAL |
| | TLSKADYEKHK | EAAGAPSVFLFPPKP | SSVVTVPSSSLG | TSGVHTFPAVL |
| | VYACEVTHQGL | KDTLMISRTPEVTCV | TQTYICNVNHKP | QSSGLYSLSSV |
| | SSPVTKSFNRGE | VVDVSHEDPEVKFN | SNTKVDKKVEP | VTVPSSSLGTQ |
| | C | WYVDGVEVHNAKT | KSCDKTHTCPPC | TYICNVNHKPS |
| | (SEQ ID NO: | KPREEQYNSTYRVVS | PAPEAAGAPSVF | NTKVDKKVEP |
| | 346) | VLTVLHQDWLNGKE | LFPPKPKDTLMIS | KSCDKTHTCPP |
| | | YKCKVSNKALPAPIE | RTPEVTCVVVD | CPAPEAAGAPS |
| | | KTISKAKGQPREPQV | VSHEDPEVKFN | VFLFPPKPKDT |
| | | YTLPPCREEMTKNQV | WYVDGVEVHN | LMISRTPEVTC |
| | | SLSCAVKGFYPSDIA | AKTKPREEQYNS | VVVDVSHEDP |
| | | VEWESNGQPENNYK | TYRVVSVLTVLH | EVKFNWYVDG |
| | | TTPPVLDSDGSFFLVS | QDWLNGKEYKC | VEVHNAKTKP |
| | | KLTVDKSRWQQGNV | KVSNKALPAPIE | REEQYNSTYR |
| | | FSCSVMHEALHNHY | KTISKAKGQPRE | VVSVLTVLHQ |
| | | TQKSLSLSPGSGGGG | PQVCTLPPSREE | DWLNGKEYKC |
| | | SGGGGSGGGGSAPTS | MTKNQVSLWCL | KVSNKALPAPI |
| | | SSTKKTQLQLEELLL | VKGFYPSDIAVE | EKTISKAKGQP |
| | | DLQMILNGINNYKNP | WESNGQPENNY | REPQVCTLPPS |
| | | KLTEMLTAKFYMPK | KTTPPVLDSDGS | REEMTKNQVS |
| | | KATELKHLQCLEEEL | FFLYSKLTVDKS | LWCLVKGFYP |
| | | KPLEEVLNLAQSKNF | RWQQGNVFSCS | SDIAVEWESNG |
| | | HLRPRDLISNINVIVL | VMHEALHNHYT | QPENNYKTTPP |
| | | ELKGSETTFMCEYAD | QKSLSLSPG | VLDSDGSFFLY |
| | | ETATIVEFLNRWITFA | (SEQ ID NO: 348) | SKLTVDKSRW |
| | | QSIISTLT | | QQGNVFSCSV |
| | | (SEQ ID NO: 347) | | MHEALHNHYT |
| | | | | QKSLSLSPGK |
| | | | | (SEQ ID NO: |
| | | | | 349) |
| xhCD8v15- | EIVLTQSPGTLS | EVQLVESGGGLVQPG | EVQLVESGGGL | EVQLVESGGG |
| IL-2m4 | LSPGERATLSCR | GSLRLSCAASGFTFSS | VQPGGSLRLSCA | LVQPGGSLRLS |
| | ASQSVSSNLAW | YAMSWVRQAPGKGL | ASGFTFSSYAMS | CAASGFTFSSY |
| | YQQKPGQAPRL | EWVSDISYAGGSTAY | WVRQAPGKGLE | AMSWVRQAPG |
| | LIYGASSRATGI | ADSVKGRFTISRDNA | WVSDISYAGGST | KGLEWVSDISY |
| | PDRFSGSGSGT | KNSLYLQMNSLRAE | AYADSVKGRFTI | AGGSTAYADS |
| | DFTLTISRLEPE | DTAVYYCARSNAYA | SRDNAKNSLYL | VKGRFTISRDN |
| | DFAVYYCQQY | WDDALDIWGQGTLV | QMNSLRAEDTA | AKNSLYLQMN |
| | GSSPPVTFGQG | TVSSASTKGPSVFPLA | VYYCARSNAYA | SLRAEDTAVY |
| | TKVEIKRTVAA | PSSKSTSGGTAALGC | WDDALDIWGQG | YCARSNAYAW |
| | PSVFIFPPSDEQ | LVKDYFPEPVTVSWN | TLVTVSSASTKG | DDALDIWGQG |
| | LKSGTASVVCL | SGALTSGVHTFPAVL | PSVFPLAPSSKST | TLVTVSSASTK |
| | LNNFYPREAKV | QSSGLYSLSSVVTVPS | SGGTAALGCLV | GPSVFPLAPSS |
| | QWKVDNALQS | SSLGTQTYICNVNHK | KDYFPEPVTVSW | KSTSGGTAAL |
| | GNSQESVTEQD | PSNTKVDKKVEPKSC | NSGALTSGVHTF | GCLVKDYFPEP |
| | SKDSTYSLSSTL | DKTHTCPPCPAPEAA | PAVLQSSGLYSL | VTVSWNSGAL |
| | TLSKADYEKHK | GAPSVFLFPPKPKDTL | SSVVTVPSSSLG | TSGVHTFPAVL |
| | VYACEVTHQGL | MISRTPEVTCVVVDV | TQTYICNVNHKP | QSSGLYSLSSV |
| | SSPVTKSFNRGE | SHEDPEVKFNWYVD | SNTKVDKKVEP | VTVPSSSLGTQ |
| | C | GVEVHNAKTKPREE | KSCDKTHTCPPC | TYICNVNHKPS |
| | (SEQ ID NO: | QYNSTYRVVSVLTVL | PAPEAAGAPSVF | NTKVDKKVEP |
| | 350) | HQDWLNGKEYKCKV | LFPPKPKDTLMIS | KSCDKTHTCPP |
| | | SNKALPAPIEKTISKA | RTPEVTCVVVD | CPAPEAAGAPS |
| | | KGQPREPQVYTLPPC | VSHEDPEVKFN | VFLFPPKPKDT |
| | | REEMTKNQVSLSCA | WYVDGVEVHN | LMISRTPEVTC |
| | | VKGFYPSDIAVEWES | AKTKPREEQYNS | VVVDVSHEDP |
| | | NGQPENNYKTTPPVL | TYRVVSVLTVLH | EVKFNWYVDG |
| | | DSDGSFFLVSKLTVD | QDWLNGKEYKC | VEVHNAKTKP |
| | | KSRWQQGNVFSCSV | KVSNKALPAPIE | REEQYNSTYR |
| | | MHEALHNHYTQKSL | KTISKAKGQPRE | VVSVLTVLHQ |
| | | SLSPGSGGGGSGGGG | PQVCTLPPSREE | DWLNGKEYKC |
| | | SGGGGSAPTSSSTKK | MTKNQVSLWCL | KVSNKALPAPI |
| | | TQLQLEELLLDLQMI | VKGFYPSDIAVE | EKTISKAKGQP |
| | | LNGINNYKNPKLTEM | WESNGQPENNY | REPQVCTLPPS |
| | | LTAKFYMPKKATEL | KTTPPVLDSDGS | REEMTKNQVS |
| | | KHLQCLEEELKPLEE | FFLYSKLTVDKS | LWCLVKGFYP |
| | | VLNLAQSKNFHLRPR | RWQQGNVFSCS | SDIAVEWESNG |
| | | DLISNINVIVLELKGS | VMHEALHNHYT | QPENNYKTTPP |
| | | ETTFMCEYADETATI | QKSLSLSPG | VLDSDGSFFLY |
| | | VEFLNRWITFAQSIIS | (SEQ ID NO: 352) | SKLTVDKSRW |
| | | TLT | | QQGNVFSCSV |
| | | (SEQ ID NO: 351) | | MHEALHNHYT |
| | | | | QKSLSLSPGK |
| | | | | (SEQ ID NO: |
| | | | | 353) |

### Properties of IL-2 Fusion Proteins

Preferential activity of the targeted IL-2 fusion proteins comprising the IL-2 polypeptides on antigen-expressing cells is demonstrated in assays that contain antigen-expressing and antigen-non expressing cells that also express IL-2Rβγ or IL-2Rαβγ. One such assay is an in vitro assay that measures STAT5 (pSTAT5) phosphorylation and/or expression of the proliferation marker Ki-67 in human immune cells, such as human peripheral blood and/or tumor-infiltrating immune cells upon exposure to IL-2 polypeptides. In one format of the assay, the activity of the targeted IL-2 fusion protein is measured on antigen-expressing and non-expressing cells to demonstrate the selectivity on antigen-expressing cells. In another format of the assay, the activity of the targeted IL-2 fusion protein comprising the IL-2 polypeptide on antigen-expressing cells is compared to that of the untargeted IL-2 fusion protein comprising the same IL-2 polypeptide and a control antibody not recognizing any antigens on antigen-expressing cells to demonstrate the magnitude of rescue in signaling of the IL-2 polypeptide when fused to an antigen binding molecule.

In some embodiments, the fusion protein of the present disclosure containing CD8b antigen binding molecules activates CD8b+ IL-2Rβ+ cells over CD8b− IL-2Rβ+ cells by at least 10-fold, at least 50-fold, or at least 100-fold. In some embodiments, said fusion protein activates CD8b+ IL-2Rβ+ cells more than 50-fold, 100 fold, or 200 fold compared to a fusion molecule comprising the said IL-2 mutant polypeptide and a control antibody not binding to any antigens expressed on said cells. Said cell activation by the IL-2 fusion protein is determined by measuring the expression of pSTAT5 or the cell proliferation marker Ki67 in said cells following the treatment with said IL-2 fusion protein.

In some embodiments, a fusion protein of the present disclosure displays one or more of the following: binds human CD8 and does not block an interaction of CD8 with MHC class I; and activates CD8+ T cells with at least 10-fold, 25-fold, 50-fold, 100-fold, 250-fold, 500-fold, or 1000-fold greater potency, e.g., as compared to activation of NK cells. In some embodiments, whether an anti-CD8 antibody or fusion protein of the present disclosure blocks the interaction of CD8 with MHC class I can be assayed, e.g., by assaying activation status of CD8+ T cells (e.g., upon antigen stimulation) in the presence or absence of the anti-CD8 antibody or fusion protein. In some embodiments, activation of CD8+ T cells and/or NK cells can be measured, e.g., by assaying one or more markers (e.g., proportion of treated cells expressing one or more markers) of proliferation (e.g., Ki67), IL-2Rβ/γ downstream signaling, and/or STAT5 downstream signaling.

### IL-21 Polypeptides

Interleukin-21 (IL-21) is a cytokine that can be expressed by T cells, B cells, NK cells and myeloid cells, and regulates the activity of both innate and adaptive immune cells and improves T cell survival and effector function. Several Phase I and II clinical trials include IL-21 as the investigational product for the treatment of cancers, inflammatory diseases, and autoimmune diseases, including, melanoma, renal cell carcinoma, acute myeloid leukemia, non-Hodgkin's lymphoma, ovarian cancer, colorectal cancer, systemic lupus erythematosus, Crohn's disease and rheumatoid arthritis.

IL-21 has a four-helix bundle structure and exists as a monomer. In humans, two isoforms of IL-21 are known, each of which are derived from a precursor molecule. The first IL-21 isoform comprises 162 amino acids (aa), the first 29 of which make up the signal peptide; and the second IL-21 isoform comprises 153 aa, the first 29 of which make up the signal peptide as in the first isoform.

IL-21 binds to the heterodimeric IL-21 receptor complex, comprising of an IL-21 receptor (IL-21R) and common gamma chain (γc). IL-21 receptor complex is expressed on the surface of T, B, and NK cells. IL-21 receptor complex is similar in structure to the IL-2 receptor complex, in that each of these cytokine receptor complex comprises a γc.

When IL-21 binds to IL-21 receptor complex, the JAK/STAT signaling pathway is activated to activate target genes. While IL-21-induced signaling may be therapeutically desirable, careful consideration of the timing and the location of the signaling is needed, given IL-21's broad expression profile and due to the fact that IL-21 has the ability to potentiate CD8+ T cell responses as well as to suppress antigen presentation and T cell priming.

The present disclosure provides, in some embodiments, IL-21 polypeptides comprising at least one amino acid substitution, relative to the human IL-21 amino acid sequence, which is provided herein as SEQ ID NO: 390. In some embodiments, “wild-type IL-21” and “human IL-21” are used interchangeably and refer to the amino acid sequence of SEQ ID NO: 390. Such IL-21 polypeptides comprising at least one amino acid substitution relative to SEQ ID NO: 390 are also referred to herein as IL-21 muteins. In some embodiments, an IL-21 polypeptide as described herein, comprises at least one and not more than X amino acid substitutions, wherein X is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or greater. In some embodiments, an IL-21 polypeptide as described herein comprises at least 35 amino acid substitutions compared to SEQ ID NO: 390. In some embodiments, an IL-21 polypeptide as described herein comprises an amino acid sequence which differs from the amino acid sequence of human IL-21 (SEQ ID NO: 390) by 10 amino acids, 15 amino acids, 20 amino acids, or 25 amino acids. In some embodiments, an IL-21 polypeptide as described herein comprises an amino acid sequence which differs from the amino acid sequence of human IL-21 (SEQ ID NO: 390) by no more than 16 amino acids. In some embodiments, an IL-21 polypeptide as described herein comprises an amino acid sequence which differs from the amino acid sequence of human IL-21 (SEQ ID NO: 390) by 4, 5, 6, 7, 8, 9, 10, 11, 12, 13, 14, 15 or 16 amino acids. In some embodiments, an IL-21 polypeptide as described herein comprises an amino acid sequence which differs from the amino acid sequence of human IL-21 (SEQ ID NO: 390) by no more than 7 amino acids or no more than 5 amino acids. In some embodiments, an IL-21 polypeptide or functional fragment or variant thereof, as described herein, comprises an amino acid sequence which differs from the amino acid sequence of human IL-21 (SEQ ID NO: 390) by 10, 11, 12, 13, 14, 15 or 16 amino acids. In some embodiments, an IL-21 polypeptide as described herein comprises an amino acid sequence which differs from the amino acid sequence of human IL-21 (SEQ ID NO: 390) by 3, 4, 5, or 6 amino acids. In some embodiments, an IL-21 polypeptide as described herein comprises an amino acid sequence which differs from the amino acid sequence of human IL-21 (SEQ ID NO: 390) by 5 to 16. In some embodiments, an IL-21 polypeptide as described herein comprises an amino acid sequence which differs from the amino acid sequence of human IL-21 (SEQ ID NO: 390) by 3 to 6 amino acids or 1 to 5 amino acids. In some embodiments, an IL-21 described herein comprises an amino acid sequence which differs from the amino acid sequence of human IL-21 (SEQ ID NO: 390) by one or two amino acids.

### Altered Isoelectric Point or Charge Distribution

In some embodiments, an IL-21 polypeptide comprises at least one mutation that alters the isoelectric point with or without affecting its binding to IL-21R. In some embodiments, an IL-21 polypeptide comprises at least one mutation that alters the isoelectric point without affecting binding to IL-21R. An altered isoelectric point of a protein can change the surface charge distribution, thereby affecting protein yield, non-specific binding, and the blood or circulatory half-life for a given protein. In some embodiments, the mutations in the IL-21 polypeptide decreases the theoretical isoelectric point by a given unit measured by a publicly available database ProtParam (SwissProt), which is hereby incorporated by reference.

In some embodiments, the IL-21 polypeptide has an isoelectric point difference of about 0.6 to about 5.0 units compared to the isoelectric point of SEQ ID NO: 390, which has an isoelectric point of about 9.42. In some embodiments, the IL-21 polypeptide has an isoelectric point of about 6.0 to about 9.0. In some embodiments, the IL-21 polypeptide comprises a theoretical isoelectric point of about 6 to about 9. In some embodiments, the IL-21 polypeptide comprises a theoretical isoelectric point of at least about 6. In some embodiments, the IL-21 polypeptide comprises a theoretical isoelectric point of at most about 9. In some embodiments, the IL-21 polypeptide comprises a theoretical isoelectric point of about 9 to about 8.9, about 9 to about 8.8, about 9 to about 8.7, about 9 to about 8.5, about 9 to about 8.3, about 9 to about 8.1, about 9 to about 7.9, about 9 to about 7.7, about 9 to about 7.5, about 9 to about 7.3, about 9 to about 7, about 9 to about 6.5, about 9 to about 6.0, about 8.9 to about 8.8, about 8.9 to about 8.7, about 8.9 to about 8.5, about 8.9 to about 8.3, about 8.9 to about 8.1, about 8.9 to about 7.9, about 8.9 to about 7.7, about 8.9 to about 7.5, about 8.9 to about 7.3, about 8.9 to about 7, about 8.9 to about 6.5, about 8.9 to about 6.0, about 8.8 to about 8.7, about 8.8 to about 8.5, about 8.8 to about 8.3, about 8.8 to about 8.1, about 8.8 to about 7.9, about 8.8 to about 7.7, about 8.8 to about 7.5, about 8.8 to about 7.3, about 8.8 to about 7, about 8.8 to about 6.5, about 8.8 to about 6.0, about 8.7 to about 8.6, about 8.7 to about 8.5, about 8.7 to about 8.3, about 8.7 to about 8.1, about 8.7 to about 7.9, about 8.7 to about 7.7, about 8.7 to about 7.5, about 8.7 to about 7.3, about 8.7 to about 7, about 8.7 to about 6.5, about 8.7 to about 6.0, about 8.5 to about 8.3, about 8.5 to about 8.4, about 8.5 to about 8.39, about 8.5 to about 8.1, about 8.5 to about 7.9, about 8.5 to about 7.7, about 8.5 to about 7.5, about 8.5 to about 7.3, about 8.5 to about 7, about 8.5 to about 6.5, about 8.5 to about 6.0, about 8.39, about 8.3 to about 8.1, about 8.3 to about 7.9, about 8.3 to about 7.7, about 8.3 to about 7.5, about 8.3 to about 7.3, about 8.3 to about 7, about 8.3 to about 6.5, about 8.3 to about 6.0, about 8.1 to about 7.9, about 8.1 to about 7.7, about 8.1 to about 7.5, about 8.1 to about 7.3, about 8.1 to about 7, about 8.1 to about 6.5, about 8.1 to about 6.0, about 7.9 to about 7.7, about 7.9 to about 7.5, about 7.9 to about 7.3, about 7.9 to about 7, about 7.9 to about 6.5, about 7.9 to about 6.0, about 7.7 to about 7.5, about 7.7 to about 7.3, about 7.7 to about 7, about 7.5 to about 7.3, about 7.5 to about 7, or about 7.3 to about 7, about 7.3 to about 6.5, about 7.3 to about 6.0, about 7.0 to about 6.5, about 7.0 to about 6.0, or about 6.5 to about 6.0. In some embodiments, the IL-21 polypeptide or a functional fragment or a variant thereof comprises a theoretical isoelectric point of about 9, about 8.9, about 8.8, about 8.79, about 8.7, about 8.5, about 8.39, about 8.3, about 8.1, about 7.9, about 7.7, about 7.5, about 7.3, about 7, about 6.5 or about 6.0. In some embodiments, the IL-21 polypeptide has a theoretical isoelectric point of less than 9.0. In some embodiments, the IL-21 polypeptide has a theoretical isoelectric point of less than 8.9, less than 8.8, less than 8.7, less than 8.5, less than 8.3, less than 8.1, less than 7.9, less than 7.7, less than 7.5, less than 7.3, less than 7.2, less than 7.0, less than 6.5 or less than 6.2.

In some instances, an altered isoelectric point can increase the protein yield of IL-21 polypeptide during the purification compared to human IL-21. In some embodiments, the increased protein yield is at least 5% greater than human IL-21. In some embodiments, the increased protein yield is at least 5%, 10%, 15%, 20%, 25%, 50%, 75%, 100%, 125%, 150%, 175%, 200%, 225%, 250%, 275%, or 300% greater than wild-type IL-21. In some embodiments, the increased protein yield is at least 5%, 10%, 15%, 20%, 25%, 50%, 75%, 100%, 125%, 150%, 175%, 200%, 225%, 250%, 275%, or 300% greater than human IL-21. In some embodiments, the increased protein yield is 5% to 10%, 5% to 15%, 5% to 20%, 5% to 25%, 5% to 50%, 5% to 75%, 5% to 100%, 5% to 125%, 5% to 150%, 5% to 175%, 5% to 200%, 5% to 225%, 5% to 250%, 5% to 275%, 5% to 300%, 10% to 15%, 10% to 20%, 10% to 25%, 10% to 50%, 10% to 75%, 10% to 100%, 10% to 125%, 10% to 150%, 10% to 175%, 10% to 200%, 10% to 225%, 10% to 250%, 10% to 275%, 10% to 300%, 15% to 20%, 15% to 25%, 15% to 50%, 15% to 75%, 15% to 100%, 15% to 125%, 15% to 150%, 15% to 175%, 15% to 200%, 15% to 225%, 15% to 250%, 15% to 275%, 15% to 300%, 20% to 25%, 20% to 50%, 20% to 75%, 20% to 100%, 20% to 125%, 20% to 150%, 20% to 175%, 20% to 200%, 20% to 225%, 20% to 250%, 20% to 275%, 20% to 300%, 25% to 50%, 25% to 75%, 25% to 100%, 25% to 125%, 25% to 150%, 25% to 175%, 25% to 200%, 25% to 225%, 25% to 250%, 25% to 275%, 25% to 300%, 50% to 75%, 50% to 100%, 50% to 125%, 50% to 150%, 50% to 175%, 50% to 200%, 75% to 100%, 75% to 125%, 75% to 150%, 75% to 175%, 75% to 200%, 75% to 225%, 75% to 250%, 75% to 275%, 75% to 300%, 100% to 125%, 100% to 150%, 100% to 175%, 100% to 200%, 100% to 225%, 100% to 250%, 100% to 275%, 100% to 300%, 125% to 150%, 125% to 175%, 125% to 200%, 125% to 225%, 125% to 250%, 125% to 275%, 125% to 300%, 150% to 175%, 150% to 200%, 175% to 200%, 175% to 225%, 175% to 250%, 175% to 275%, 175% to 300%, 200% to 225%, 200% to 250%, 200% to 275%, 200% to 300%, 225% to 250%, 225% to 275%, 225% to 300%, 250% to 275%, 250% to 300%, or 275% to 300%. In some embodiments, the increased protein yield is about 5%, 10%, 15%, 20%, 25%, 50%, 75%, 100%, 125%, 150%, 175%, 200%, 225%, 250%, 275%, or 300% greater than human IL-21.

In some embodiments, human IL-21 comprises a region of 2-20 positively charged amino acid residues. In some embodiments, the region is 2-3, 2-4, 2-5, 2-7, 2-8, 2-9, 2-10, 2-11, 2-12, 2-13, 2-14, 2-15, 2-16, 2-17, 2-18, 2-19, 2-20, 3-4, 3-5, 3-6, 3-7, 3-8, 3-9, 3-10, 3-11, 3-12, 3-13, 3-14, 3-15, 3-16, 3-17, 3-18, 3-19, 3-20, 4-5, 4-6, 4-7, 4-8, 4-9, 4-10, 4-11, 4-12, 4-13, 4-14, 4-15, 4-16, 4-17, 4-18, 4-19, 4-20, 5-6, 5-7, 5-8, 5-9, 5-10, 5-11, 5-12, 5-13, 5-14, 5-15, 5-16, 5-17, 5-18, 5-19, 5-20, 6-7, 6-8, 6-9, 6-10, 6-11, 6-12, 6-13, 6-14, 6-15, 6-16, 6-17, 6-18, 6-19, 6-20, 7-8, 7-9, 7-10, 7-11, 7-12, 7-13, 7-14, 7-15, 7-16, 7-17, 7-18, 7-19, 7-20, 8-9, 8-10, 8-11, 8-12, 8-13, 8-14, 8-15, 8-16, 8-17, 8-18, 8-19, 8-20, 9-10, 9-11, 9-12, 9-13, 9-14, 9-15, 9-16, 9-17, 9-18, 9-19, 9-20, 10-11, 10-12, 10-13, 10-14, 10-15, 10-16, 10-17, 10-18, 10-19, 10-20, 11-12, 11-13, 11-14, 11-15, 11-16, 11-17, 11-18, 11-19, 11-20, 12-13, 12-14, 12-15, 12-16, 12-17, 12-18, 12-19, 12-20, 13-14, 13-15, 13-16, 13-17, 13-18, 13-19, 13-20, 14-15, 14-16, 14-17, 3-18, 14-19, 14-20, 15-16, 15-17, 15-18, 15-19, 15-20, 16-17, 16-18, 16-19, 16-20, 17-18, 17-19, 17-20 18-19, 18-20, or 19-20 positively charged amino acid residues. In some embodiments, human IL-21 comprises a region of 12 positively charged amino acid residues. In some embodiments, human IL-21 comprises a region of 2-20 amino acid residues wherein at least one amino acid is positively charged. In some embodiments, the region is 2-3, 2-4, 2-5, 2-7, 2-8, 2-9, 2-10, 2-11, 2-12, 2-13, 2-14, 2-15, 2-16, 2-17, 2-18, 2-19, 2-20, 3-4, 3-5, 3-6, 3-7, 3-8, 3-9, 3-10, 3-11, 3-12, 3-13, 3-14, 3-15, 3-16, 3-17, 3-18, 3-19, 3-20, 4-5, 4-6, 4-7, 4-8, 4-9, 4-10, 4-11, 4-12, 4-13, 4-14, 4-15, 4-16, 4-17, 4-18, 4-19, 4-20, 5-6, 5-7, 5-8, 5-9, 5-10, 5-11, 5-12, 5-13, 5-14, 5-15, 5-16, 5-17, 5-18, 5-19, 5-20, 6-7, 6-8, 6-9, 6-10, 6-11, 6-12, 6-13, 6-14, 6-15, 6-16, 6-17, 6-18, 6-19, 6-20, 7-8, 7-9, 7-10, 7-11, 7-12, 7-13, 7-14, 7-15, 7-16, 7-17, 7-18, 7-19, 7-20, 8-9, 8-10, 8-11, 8-12, 8-13, 8-14, 8-15, 8-16, 8-17, 8-18, 8-19, 8-20, 9-10, 9-11, 9-12, 9-13, 9-14, 9-15, 9-16, 9-17, 9-18, 9-19, 9-20, 10-11, 10-12, 10-13, 10-14, 10-15, 10-16, 10-17, 10-18, 10-19, 10-20, 11-12, 11-13, 11-14, 11-15, 11-16, 11-17, 11-18, 11-19, 11-20, 12-13, 12-14, 12-15, 12-16, 12-17, 12-18, 12-19, 12-20, 13-14, 13-15, 13-16, 13-17, 13-18, 13-19, 13-20, 14-15, 14-16, 14-17, 3-18, 14-19, 14-20, 15-16, 15-17, 15-18, 15-19, 15-20, 16-17, 16-18, 16-19, 16-20, 17-18, 17-19, 17-20 18-19, 18-20, or 19-20 amino acid residues with at least 2, 3, 4, 5, 6, 7, 8, 9, 10, 11, 12, 13, 14, 15, 16, 17, 18, 19 or 20 positively charged amino acid residues. In some embodiments, human IL-21 comprises a region of 12 amino acid residues wherein 2, 3, 4, 5, 6, 7, 8, 9, 10, 11 or 12 amino acid residues are positively charged. In some embodiments, human IL-21 comprises a region of 12 amino acid residues wherein 2, 3, 4, 5, or 6 amino acid residues are positively charged.

In some embodiments, amino acids within the region do not bind a human IL-21 receptor. In some embodiments, the region comprises amino acid residues S80-T92 of SEQ ID NO: 390. In some embodiments, the region comprises positively charged amino acid residues R85, R86, K88, H89, and R90. In some embodiments, the IL-21 polypeptide comprises at least one amino acid substitution in the region of human IL-21. In some embodiments, the IL-21 polypeptide comprises at least one amino acid substitution of S80-T92. In some embodiments, the IL-21 polypeptide comprises at least four amino acid substitutions of S80-T92. In some embodiments, the IL-21 polypeptide comprises at least four amino acid substitutions of S80-T92, provided G84 is not substituted. In some embodiments, the IL-21 polypeptide comprises at least one amino acid substitution at R85, R86, K88, H89 or R90. In some embodiments, the IL-21 polypeptide comprises an amino acid substitution at one or more of S80, T81, N82, A83, R85, R86, Q87, K88, H89, R90, L91 and T92. In some embodiments, the IL-21 polypeptide comprises amino acid substitutions at S80, T81, N82, A83, R85, R86, Q87, K88, H89, R90, L91 and T92.

In some embodiments, the IL-21 polypeptides of the present disclosure have one or more, two or more, or three or more charge-reducing amino acid substitutions relative to the wild-type mature IL-21 polypeptide, e.g., having the amino acid sequence of SEQ ID NO: 390. In some embodiments, one or more, two or more, or three or more substituted residues, are selected from the following group: of K56, S80, T81, N82, A83, G84, R85, R86, Q87, K88, H89, R90, L91, and T92.

In some embodiments, the IL-21 polypeptide that comprises an altered isoelectric point relative to human IL-21 (SEQ ID NO: 390) comprises a mutation in a position selected from the group consisting of K56, S80, T81, N82, A83, G84, R85, R86, Q87, K88, H89, R90, L91, and T92 of SEQ ID NO: 390. In some embodiments, the IL-21 polypeptide that comprises an altered isoelectric point relative to human IL-21 (SEQ ID NO: 390) comprises a mutation in a position selected from the group consisting of S80, T81, N82, A83, G84, R85, R86, Q87, K88, H89, R90, L91, and T92 of SEQ ID NO: 390. In some embodiments, the IL-21 polypeptide that comprises an altered isoelectric point relative to human IL-21 (SEQ ID NO: 390) comprises a mutation in a position selected from the group consisting of S80, T81, N82, A83, R85, R86, Q87, K88, H89, R90, L91, and T92 of SEQ ID NO: 390.

In some embodiments, the IL-21 polypeptide comprises a mutation at position K56 of SEQ ID NO: 390. In some embodiments, the mutation comprises K56G, K56S, K56E, K56D, or K56A. In some embodiments, the IL-21 polypeptide comprises a mutation at position S80. In some embodiments, the mutation comprises S80G, S80A, S80D or S80E. In some embodiments, the IL-21 polypeptide comprises a mutation at position T81 of SEQ ID NO: 390. In some embodiments, the mutation comprises T81G, T81S, T81E, T81D, or T81A. In some embodiments, the IL-21 polypeptide comprises a mutation at position N82 of SEQ ID NO: 390. In some embodiments, the mutation comprises N82G, N82S, N82E, N82D, or N82A. In some embodiments, the IL-21 polypeptide comprises a mutation at position A83 of SEQ ID NO: 390. In some embodiments, the mutation comprises A83G, A83S, A83E, or A83D. In some embodiments, the IL-21 polypeptide comprises a mutation at position G84 of SEQ ID NO: 390. In some embodiments, the mutation comprises G84A, G84S, G84E, or G84D. In some embodiments, G84 is not mutated. In some embodiments, the IL-21 polypeptide comprises a mutation at position R85 of SEQ ID NO: 390. In some embodiments, the mutation comprises R85G, R85S, R85E, R85D, or R85A. In some embodiments, the IL-21 polypeptide comprises a mutation at position R86 of SEQ ID NO: 390. In some embodiments, the mutation comprises R86G, R86S, R86E, R86D, or R86A. In some embodiments, the IL-21 polypeptide comprises a mutation at position Q87 of SEQ ID NO: 390. In some embodiments, the mutation comprises Q87G, Q87S, Q87E, Q87D, or Q87A. In some embodiments, the IL-21 polypeptide comprises a mutation at position K88 of SEQ ID NO: 390. In some instances, the mutation comprises K88G, K88S, K88E, K88D, or K88A. In some embodiments, the IL-21 polypeptide comprises a mutation at position H89 of SEQ ID NO: 390. In some embodiments, the mutation comprises H89G, H89S, H89E, H89D, or H89A. In some embodiments, the IL-21 polypeptide comprises a mutation at position R90 of SEQ ID NO: 390. In some embodiments, the mutation comprises R90G, R90S, R90E, R90D, or R90A. In some embodiments, the IL-21 polypeptide comprises a mutation at position L91 of SEQ ID NO: 390. In some embodiments, the mutation comprises L91G, L91S, L91E, L91D, or L91A. In some embodiments, the IL-21 polypeptide comprises a mutation at position T92 of SEQ ID NO: 390. In some embodiments, the mutation comprises T92G, T92S, T92E, T92D, or T92A.

In some embodiments, an IL-21 polypeptide comprises SEQ ID NO: 390 with one of the following sets of amino acid substitutions (relative to SEQ ID NO: 390): G85, G86, G88, and A90; G85, G86, G88, and E90; A56, A75, G85, G86, G88, and E90; A56, E75, G85, G86, G88, and A90; E56, A75, G85, G86, G88, and A90; G80, G81, G82, S83, E85, G86, S87, G88, G89, and S90; G80, G81, G82, S83, G85, G86, S87, G88, G89, S90; G85, G86, S87, G88, G89, and E90; G85, G86, S87, G88, G89, and S90; G85, G86, G87, G88, G89, and E90; G85, G86, G87, G88, G89, and G90; G85, G86, E87, G88, G89, and G90; G80, G81, G82, S83, G85, G86, S87, G88, G89, G90, S91 and G92; G80, G81, G82, G83, G85, G86, G87, G88, G89, G90, G91 and G92; G80, G81, G82, S83, G85, G86, E87, G88, G89, G90, S91 and G92; G80, G81, G82, E83, G85, G86, E87, G88, G89, G90, S91 and G92; G82, G83, S85, G86, G87, G88, S89, G90, G91 and S92; G82, G83, E85, G86, G87, G88, S89, G90, G91 and S92; G80, G81, G82, G83, E85, G86, G87, G88, G89, G90, G91 and G92; G80, G81, G82, S83, G85, G86, S87, G88, G89, E90, G91 and G92; G80, G81, G82, G83, G85, G86, G87, G88, G89, E90, G91 and G92; G82, G83, S85, G86, G87, G88, S89, E90, G91 and S92; G82, G83, G85, G86, G87, G88, G89, E90, G91 and G92; G85, G86, G87, G88, G89, E90, and G91; G82, G83, G85, G86, G87, G88, G89, E90, and G91; G82, G83, G85, E86, G87, G88, G89, E90, and G91; G82, G83, E85, G86, G87, G88, G89, E90, and G91; G80, G81, G82, G83, E84, G85, G86, G87, G88, G89, E90, G91 and G92; G80, G81, G82, E83, G85, G86, G87, G88, G89, E90, G91 and G92; or G80, G81, E82, G83, G85, G86, G87, G88, G89, E90, G91 and G92.

In some embodiments, an IL-21 polypeptide comprises at least 80% sequence identity to SEQ ID NO: 390 and comprises amino acids G85, G86, G88, and A90. In some embodiments, an IL-21 polypeptide comprises at least 85%, 90%, 95%, 96%, 97%, 98%, 99% or 100% sequence identity to SEQ ID NO: 390 and comprises amino acids G85, G86, G88, and A90. In some embodiments, an IL-21 polypeptide comprises at least 80% sequence identity to SEQ ID NO: 390 and comprises amino acids G85, G86, G88, and E90. In some embodiments, an IL-21 polypeptide comprises at least 85%, 90%, 95%, 96%, 97%, 98%, 99% or 100% sequence identity to SEQ ID NO: 390 and comprises amino acids G85, G86, G88, and E90. In some embodiments, an IL-21 polypeptide comprises at least 80% sequence identity to SEQ ID NO: 390 and comprises amino acids A56, A75, G85, G86, G88, and E90. In some embodiments, an IL-21 polypeptide comprises at least 85%, 90%, 95%, 96%, 97%, 98%, 99% or 100% sequence identity to SEQ ID NO: 390 and comprises amino acids A56, A75, G85, G86, G88, and E90. In some embodiments, an IL-21 polypeptide comprises at least 80% sequence identity to SEQ ID NO: 390 and comprises amino acids: A56, E75, G85, G86, G88, and A90. In some embodiments, an IL-21 polypeptide comprises at least 85%, 90%, 95%, 96%, 97%, 98%, 99% or 100% sequence identity to SEQ ID NO: 390 and comprises amino acids A56, E75, G85, G86, G88, and A90. In some embodiments, an IL-21 polypeptide comprises at least 80% sequence identity to SEQ ID NO: 390 and comprises amino acids: A56, A75, G85, G86, G88, and A90. In some embodiments, an IL-21 polypeptide comprises at least 85%, 90%, 95%, 96%, 97%, 98%, 99% or 100% sequence identity to SEQ ID NO: 390 and comprises amino acids A56, A75, G85, G86, G88, and A90. In some embodiments, an IL-21 polypeptide comprises at least 80% sequence identity to SEQ ID NO: 390 and comprises amino acids: E56, A75, G85, G86, G88, and A90. In some embodiments, an IL-21 polypeptide comprises at least 85%, 90%, 95%, 96%, 97%, 98%, 99% or 100% sequence identity to SEQ ID NO: 390 and comprises amino acids E56, A75, G85, G86, G88, and A90. In some embodiments, an IL-21 polypeptide comprises at least 80% sequence identity to SEQ ID NO: 390 and comprises amino acids: G80, G81, G82, S83, E85, G86, S87, G88, G89, and S90. In some embodiments, an IL-21 polypeptide comprises at least 85%, 90%, 95%, 96%, 97%, 98%, 99% or 100% sequence identity to SEQ ID NO: 390 and comprises amino acids G80, G81, G82, S83, E85, G86, S87, G88, G89, and S90. In some embodiments, an IL-21 polypeptide comprises at least 80% sequence identity to SEQ ID NO: 390 and comprises amino acids: G80, G81, G82, S83, G85, G86, S87, G88, G89, S90. In some embodiments, an IL-21 polypeptide comprises at least 85%, 90%, 95%, 96%, 97%, 98%, 99% or 100% sequence identity to SEQ ID NO: 390 and comprises amino acids G80, G81, G82, S83, G85, G86, S87, G88, G89, S90. In some embodiments, an IL-21 polypeptide comprises at least 80% sequence identity to SEQ ID NO: 390 and comprises amino acids: G80, G81, G82, S83, G85, G86, S87, G88, G89, E90. In some embodiments, an IL-21 polypeptide comprises at least 85%, 90%, 95%, 96%, 97%, 98%, 99% or 100% sequence identity to SEQ ID NO: 390 and comprises amino acids G80, G81, G82, S83, G85, G86, S87, G88, G89, E90. In some embodiments, an IL-21 polypeptide comprises at least 80% sequence identity to SEQ ID NO: 390 and comprises amino acids: G85, G86, S87, G88, G89, and E90. In some embodiments, an IL-21 polypeptide comprises at least 85%, 90%, 95%, 96%, 97%, 98%, 99% or 100% sequence identity to SEQ ID NO: 390 and comprises amino acids G85, G86, S87, G88, G89, and E90. In some embodiments, an IL-21 polypeptide comprises at least 80% sequence identity to SEQ ID NO: 390 and comprises amino acids: G85, G86, S87, G88, G89, and S90. In some embodiments, an IL-21 polypeptide comprises at least 85%, 90%, 95%, 96%, 97%, 98%, 99% or 100% sequence identity to SEQ ID NO: 390 and comprises amino acids G85, G86, S87, G88, G89, and S90. In some embodiments, an IL-21 polypeptide comprises at least 80% sequence identity to SEQ ID NO: 390 and comprises amino acids: G85, G86, G87, G88, G89, and E90. In some embodiments, an IL-21 polypeptide comprises at least 85%, 90%, 95%, 96%, 97%, 98%, 99% or 100% sequence identity to SEQ ID NO: 390 and comprises amino acids G85, G86, G87, G88, G89, and E90. In some embodiments, an IL-21 polypeptide comprises at least 80% sequence identity to SEQ ID NO: 390 and comprises amino acids: G85, G86, G87, G88, G89, and G90. In some embodiments, an IL-21 polypeptide comprises at least 85%, 90%, 95%, 96%, 97%, 98%, 99% or 100% sequence identity to SEQ ID NO: 390 and comprises amino acids G85, G86, G87, G88, G89, and G90. In some embodiments, an IL-21 polypeptide comprises at least 80% sequence identity to SEQ ID NO: 390 and comprises amino acids: G85, G86, E87, G88, G89, and G90. In some embodiments, an IL-21 polypeptide comprises at least 85%, 90%, 95%, 96%, 97%, 98%, 99% or 100% sequence identity to SEQ ID NO: 390 and comprises amino acids G85, G86, E87, G88, G89, and G90.

In some embodiments, an IL-21 polypeptide comprises at least 80% sequence identity to SEQ ID NO: 390 and comprises amino acids: G80, G81, G82, S83, G85, G86, S87, G88, G89, G90, S91 and G92. In some embodiments, an IL-21 polypeptide comprises at least 85%, 90%, 95%, 96%, 97%, 98%, 99% or 100% sequence identity to SEQ ID NO: 390 and comprises amino acids G80, G81, G82, S83, G85, G86, S87, G88, G89, G90, S91 and G92. In some embodiments, an IL-21 polypeptide comprises at least 80% sequence identity to SEQ ID NO: 390 and comprises amino acids: G80, G81, G82, G83, G85, G86, G87, G88, G89, G90, G91 and G92. In some embodiments, an IL-21 polypeptide comprises at least 85%, 90%, 95%, 96%, 97%, 98%, 99% or 100% sequence identity to SEQ ID NO: 390 and comprises amino acids G80, G81, G82, G83, G85, G86, G87, G88, G89, G90, G91 and G92. In some embodiments, an IL-21 polypeptide comprises at least 80% sequence identity to SEQ ID NO: 390 and comprises amino acids: G80, G81, G82, S83, G85, G86, E87, G88, G89, G90, S91 and G92. In some embodiments, an IL-21 polypeptide comprises at least 85%, 90%, 95%, 96%, 97%, 98%, 99% or 100% sequence identity to SEQ ID NO: 390 and comprises amino acids G80, G81, G82, S83, G85, G86, E87, G88, G89, G90, S91 and G92. In some embodiments, an IL-21 polypeptide comprises at least 80% sequence identity to SEQ ID NO: 390 and comprises amino acids: G80, G81, G82, E83, G85, G86, E87, G88, G89, G90, S91 and G92. In some embodiments, an IL-21 polypeptide comprises at least 85%, 90%, 95%, 96%, 97%, 98%, 99% or 100% sequence identity to SEQ ID NO: 390 and comprises amino acids G80, G81, G82, E83, G85, G86, E87, G88, G89, G90, S91 and G92. In some embodiments, an IL-21 polypeptide comprises at least 80% sequence identity to SEQ ID NO: 390 and comprises amino acids: G82, G83, S85, G86, G87, G88, S89, G90, G91 and S92. In some embodiments, an IL-21 polypeptide comprises at least 85%, 90%, 95%, 96%, 97%, 98%, 99% or 100% sequence identity to SEQ ID NO: 390 and comprises amino acids G82, G83, S85, G86, G87, G88, S89, G90, G91 and S92. In some embodiments, an IL-21 polypeptide comprises at least 80% sequence identity to SEQ ID NO: 390 and comprises amino acids: G82, G83, E85, G86, G87, G88, S89, G90, G91 and S92. In some embodiments, an IL-21 polypeptide comprises at least 85%, 90%, 95%, 96%, 97%, 98%, 99% or 100% sequence identity to SEQ ID NO: 390 and comprises amino acids G82, G83, E85, G86, G87, G88, S89, G90, G91 and S92. In some embodiments, an IL-21 polypeptide comprises at least 80% sequence identity to SEQ ID NO: 390 and comprises amino acids: G80, G81, G82, G83, E85, G86, G87, G88, G89, G90, G91 and G92. In some embodiments, an IL-21 polypeptide comprises at least 85%, 90%, 95%, 96%, 97%, 98%, 99% or 100% sequence identity to SEQ ID NO: 390 and comprises amino acids G80, G81, G82, G83, E85, G86, G87, G88, G89, G90, G91 and G92. In some embodiments, an IL-21 polypeptide comprises at least 80% sequence identity to SEQ ID NO: 390 and comprises amino acids: G80, G81, G82, S83, G85, G86, S87, G88, G89, E90, G91 and G92. In some embodiments, an IL-21 polypeptide comprises at least 85%, 90%, 95%, 96%, 97%, 98%, 99% or 100% sequence identity to SEQ ID NO: 390 and comprises amino acids G80, G81, G82, S83, G85, G86, S87, G88, G89, E90, G91 and G92. In some embodiments, an IL-21 polypeptide comprises at least 80% sequence identity to SEQ ID NO: 390 and comprises amino acids: G80, G81, G82, G83, G85, G86, G87, G88, G89, E90, G91 and G92. In some embodiments, an IL-21 polypeptide comprises at least 85%, 90%, 95%, 96%, 97%, 98%, 99% or 100% sequence identity to SEQ ID NO: 390 and comprises amino acids G80, G81, G82, G83, G85, G86, G87, G88, G89, E90, G91 and G92. In some embodiments, an IL-21 polypeptide comprises at least 80% sequence identity to SEQ ID NO: 390 and comprises amino acids: G82, G83, S85, G86, G87, G88, S89, E90, G91 and S92. In some embodiments, an IL-21 polypeptide comprises at least 85%, 90%, 95%, 96%, 97%, 98%, 99% or 100% sequence identity to SEQ ID NO: 390 and comprises amino acids G82, G83, S85, G86, G87, G88, S89, E90, G91 and S92. In some embodiments, an IL-21 polypeptide comprises at least 80% sequence identity to SEQ ID NO: 390 and comprises amino acids: G82, G83, G85, G86, G87, G88, G89, E90, G91 and G92. In some embodiments, an IL-21 polypeptide comprises at least 85%, 90%, 95%, 96%, 97%, 98%, 99% or 100% sequence identity to SEQ ID NO: 390 and comprises amino acids G82, G83, G85, G86, G87, G88, G89, E90, G91 and G92. In some embodiments, an IL-21 polypeptide comprises at least 80% sequence identity to SEQ ID NO: 390 and comprises amino acids: G85, G86, G87, G88, G89, E90, and G91. In some embodiments, an IL-21 polypeptide comprises at least 85%, 90%, 95%, 96%, 97%, 98%, 99% or 100% sequence identity to SEQ ID NO: 390 and comprises amino acids G85, G86, G87, G88, G89, E90, and G91. In some embodiments, an IL-21 polypeptide comprises at least 80% sequence identity to SEQ ID NO: 390 and comprises amino acids: G82, G83, G85, G86, G87, G88, G89, E90, and G91. In some embodiments, an IL-21 polypeptide comprises at least 85%, 90%, 95%, 96%, 97%, 98%, 99% or 100% sequence identity to SEQ ID NO: 390 and comprises amino acids G82, G83, G85, G86, G87, G88, G89, E90, and G91. In some embodiments, an IL-21 polypeptide comprises at least 80% sequence identity to SEQ ID NO: 390 and comprises amino acids: G82, G83, G85, E86, G87, G88, G89, E90, and G91. In some embodiments, an IL-21 polypeptide comprises at least 85%, 90%, 95%, 96%, 97%, 98%, 99% or 100% sequence identity to SEQ ID NO: 390 and comprises amino acids G82, G83, G85, E86, G87, G88, G89, E90, and G91. In some embodiments, an IL-21 polypeptide comprises at least 80% sequence identity to SEQ ID NO: 390 and comprises amino acids: G82, G83, E85, G86, G87, G88, G89, E90, and G91. In some embodiments, an IL-21 polypeptide comprises at least 85%, 90%, 95%, 96%, 97%, 98%, 99% or 100% sequence identity to SEQ ID NO: 390 and comprises amino acids G82, G83, E85, G86, G87, G88, G89, E90, and G91. In some embodiments, an IL-21 polypeptide comprises at least 80% sequence identity to SEQ ID NO: 390 and comprises amino acids: G80, G81, G82, G83, E84, G85, G86, G87, G88, G89, E90, G91 and G92. In some embodiments, an IL-21 polypeptide comprises at least 85%, 90%, 95%, 96%, 97%, 98%, 99% or 100% sequence identity to SEQ ID NO: 390 and comprises amino acids G80, G81, G82, G83, E84, G85, G86, G87, G88, G89, E90, G91 and G92. In some embodiments, an IL-21 polypeptide comprises at least 80% sequence identity to SEQ ID NO: 390 and comprises amino acids: G80, G81, G82, E83, G85, G86, G87, G88, G89, E90, G91 and G92. In some embodiments, an IL-21 polypeptide comprises at least 85%, 90%, 95%, 96%, 97%, 98%, 99% or 100% sequence identity to SEQ ID NO: 390 and comprises amino acids G80, G81, G82, E83, G85, G86, G87, G88, G89, E90, G91 and G92. In some embodiments, an IL-21 polypeptide comprises at least 80% sequence identity to SEQ ID NO: 390 and comprises amino acids: G80, G81, E82, G83, G85, G86, G87, G88, G89, E90, G91 and G92. In some embodiments, an IL-21 polypeptide comprises at least 85%, 90%, 95%, 96%, 97%, 98%, 99% or 100% sequence identity to SEQ ID NO: 390 and comprises amino acids G80, G81, E82, G83, G85, G86, G87, G88, G89, E90, G91 and G92.

Provided in some embodiments are charge variant IL-21 polypeptides or functional fragments or variants thereof that comprise a sequence that is at least 90%, 91%, 92%, 93%, 94%, 95%, 96%, 97%, 98%, 99%, or 100% identical to a sequence selected from the group consisting of SEQ ID NOs. 391-421.

In some embodiments, an IL-21 polypeptide comprises about 75% sequence identity to about 100% sequence identity to a sequence selected from the group consisting of SEQ ID NOs. 391-421. In some embodiments, an IL-21 polypeptide comprises at least about 75% sequence identity to a sequence selected from the group consisting of SEQ ID NOs. 391-421. In some embodiments, an IL-21 polypeptide comprises at most about 100% sequence identity to a sequence selected from the group consisting of SEQ ID NOs. 391-421. In some embodiments, an IL-21 polypeptide comprises about 75% sequence identity to about 80% sequence identity, about 75% sequence identity to about 85% sequence identity, about 75% sequence identity to about 90% sequence identity, about 75% sequence identity to about 95% sequence identity, about 75% sequence identity to about 96% sequence identity, about 75% sequence identity to about 97% sequence identity, about 75% sequence identity to about 98% sequence identity, about 75% sequence identity to about 99% sequence identity, about 75% sequence identity to about 100% sequence identity, about 80% sequence identity to about 85% sequence identity, about 80% sequence identity to about 90% sequence identity, about 80% sequence identity to about 95% sequence identity, about 80% sequence identity to about 96% sequence identity, about 80% sequence identity to about 97% sequence identity, about 80% sequence identity to about 98% sequence identity, about 80% sequence identity to about 99% sequence identity, about 80% sequence identity to about 100% sequence identity, about 85% sequence identity to about 90% sequence identity, about 85% sequence identity to about 95% sequence identity, about 85% sequence identity to about 96% sequence identity, about 85% sequence identity to about 97% sequence identity, about 85% sequence identity to about 98% sequence identity, about 85% sequence identity to about 99% sequence identity, about 85% sequence identity to about 100% sequence identity, about 90% sequence identity to about 95% sequence identity, about 90% sequence identity to about 96% sequence identity, about 90% sequence identity to about 97% sequence identity, about 90% sequence identity to about 98% sequence identity, about 90% sequence identity to about 99% sequence identity, about 90% sequence identity to about 100% sequence identity, about 95% sequence identity to about 96% sequence identity, about 95% sequence identity to about 97% sequence identity, about 95% sequence identity to about 98% sequence identity, about 95% sequence identity to about 99% sequence identity, about 95% sequence identity to about 100% sequence identity, about 96% sequence identity to about 97% sequence identity, about 96% sequence identity to about 98% sequence identity, about 96% sequence identity to about 99% sequence identity, about 96% sequence identity to about 100% sequence identity, about 97% sequence identity to about 98% sequence identity, about 97% sequence identity to about 99% sequence identity, about 97% sequence identity to about 100% sequence identity, about 98% sequence identity to about 99% sequence identity, about 98% sequence identity to about 100% sequence identity, or about 99% sequence identity to about 100% sequence identity to a sequence selected from the group consisting of SEQ ID NOs. 2, 4, 5, 6, 7, 8, 9, 10, 11, 12, 13, 14 15, 23, 24, 25, 26, 27, 28, 29, 30, 31, 32, 33, 34, 35, 36, 37, 38, 39 and 40. In some embodiments, an IL-21 polypeptide comprises about 75% sequence identity, about 80% sequence identity, about 85% sequence identity, about 90% sequence identity, about 95% sequence identity, about 96% sequence identity, about 97% sequence identity, about 98% sequence identity, about 99% sequence identity, or about 100% sequence identity to a sequence selected from the group consisting of SEQ ID NOs. 391-421.

In some embodiments, an IL-21 polypeptide or a functional fragment or a variant thereof comprises at least 80% sequence identity to SEQ ID NO: 392 and/or comprises amino acids G85, G86, G88, and A90. In some embodiments, an IL-21 polypeptide or a functional fragment or a variant thereof comprises at least 85%, 90%, 95%, 96%, 97%, 98%, 99% or 100% sequence identity to SEQ ID NO: 392 and/or comprises amino acids G85, G86, G88, and A90. In some embodiments, an IL-21 polypeptide or a functional fragment or a variant thereof comprises at least 80% sequence identity to SEQ ID NO: 391 and/or comprises amino acids G85, G86, G88, and E90. In some embodiments, an IL-21 polypeptide or a functional fragment or a variant thereof comprises at least 85%, 90%, 95%, 96%, 97%, 98%, 99% or 100% sequence identity to SEQ ID NO: 391 and/or comprises amino acids G85, G86, G88, and E90. In some embodiments, an IL-21 polypeptide or a functional fragment or a variant thereof comprises at least 80% sequence identity to SEQ ID NO: 393 and/or comprises amino acids A56, A75, G85, G86, G88, and E90. In some embodiments, an IL-21 polypeptide or a functional fragment or a variant thereof comprises at least 85%, 90%, 95%, 96%, 97%, 98%, 99% or 100% sequence identity to SEQ ID NO: 393 and/or comprises amino acids A56, A75, G85, G86, G88, and E90. In some embodiments, an IL-21 polypeptide or a functional fragment or a variant thereof comprises at least 80% sequence identity to SEQ ID NO: 394 and/or comprises amino acids: A56, E75, G85, G86, G88, and A90. In some embodiments, an IL-21 polypeptide or a functional fragment or a variant thereof comprises at least 85%, 90%, 95%, 96%, 97%, 98%, 99% or 100% sequence identity to SEQ ID NO: 394 and/or comprises amino acids A56, E75, G85, G86, G88, and A90. In some embodiments, an IL-21 polypeptide or a functional fragment or a variant thereof comprises at least 80% sequence identity to SEQ ID NO: 395 and/or comprises amino acids: A56, A75, G85, G86, G88, and A90. In some embodiments, an IL-21 polypeptide or a functional fragment or a variant thereof comprises at least 85%, 90%, 95%, 96%, 97%, 98%, 99% or 100% sequence identity to SEQ ID NO: 395 and/or comprises amino acids A56, A75, G85, G86, G88, and A90. In some embodiments, an IL-21 polypeptide or a functional fragment or a variant thereof comprises at least 80% sequence identity to SEQ ID NO: 396 and/or comprises amino acids: E56, A75, G85, G86, G88, and A90. In some embodiments, an IL-21 polypeptide or a functional fragment or a variant thereof comprises at least 85%, 90%, 95%, 96%, 97%, 98%, 99% or 100% sequence identity to SEQ ID NO: 396 and/or comprises amino acids E56, A75, G85, G86, G88, and A90. In some embodiments, an IL-21 polypeptide or a functional fragment or a variant thereof comprises at least 80% sequence identity to SEQ ID NO: 397 and/or comprises amino acids: G80, G81, G82, S83, E85, G86, S87, G88, G89, and S90. In some embodiments, an IL-21 polypeptide or a functional fragment or a variant thereof comprises at least 85%, 90%, 95%, 96%, 97%, 98%, 99% or 100% sequence identity to SEQ ID NO: 397 and/or comprises amino acids G80, G81, G82, S83, E85, G86, S87, G88, G89, and S90. In some embodiments, an IL-21 polypeptide or a functional fragment or a variant thereof comprises at least 80% sequence identity to SEQ ID NO: 398 and/or comprises amino acids: G80, G81, G82, S83, G85, G86, S87, G88, G89, S90. In some embodiments, an IL-21 polypeptide or a functional fragment or a variant thereof comprises at least 85%, 90%, 95%, 96%, 97%, 98%, 99% or 100% sequence identity to SEQ ID NO: 398 and/or comprises amino acids G80, G81, G82, S83, G85, G86, S87, G88, G89, S90. In some embodiments, an IL-21 polypeptide or a functional fragment or a variant thereof comprises at least 80% sequence identity to SEQ ID NO: 398 and/or comprises amino acids: G80, G81, G82, S83, G85, G86, S87, G88, G89, E90. In some embodiments, an IL-21 polypeptide or a functional fragment or a variant thereof comprises at least 85%, 90%, 95%, 96%, 97%, 98%, 99% or 100% sequence identity to SEQ ID NO: 398 and/or comprises amino acids G80, G81, G82, S83, G85, G86, S87, G88, G89, E90. In some embodiments, an IL-21 polypeptide or a functional fragment or a variant thereof comprises at least 80% sequence identity to SEQ ID NO: 399 and/or comprises amino acids: G85, G86, S87, G88, G89, and E90. In some embodiments, an IL-21 polypeptide or a functional fragment or a variant thereof comprises at least 85%, 90%, 95%, 96%, 97%, 98%, 99% or 100% sequence identity to SEQ ID NO: 399 and/or comprises amino acids G85, G86, S87, G88, G89, and E90. In some embodiments, an IL-21 polypeptide or a functional fragment or a variant thereof comprises at least 80% sequence identity to SEQ ID NO: 400 and/or comprises amino acids: G85, G86, S87, G88, G89, and S90. In some embodiments, an IL-21 polypeptide or a functional fragment or a variant thereof comprises at least 85%, 90%, 95%, 96%, 97%, 98%, 99% or 100% sequence identity to SEQ ID NO: 400 and/or comprises amino acids G85, G86, S87, G88, G89, and S90. In some embodiments, an IL-21 polypeptide or a functional fragment or a variant thereof comprises at least 80% sequence identity to SEQ ID NO: 401 and/or comprises amino acids: G85, G86, G87, G88, G89, and E90. In some embodiments, an IL-21 polypeptide or a functional fragment or a variant thereof comprises at least 85%, 90%, 95%, 96%, 97%, 98%, 99% or 100% sequence identity to SEQ ID NO: 401 and/or comprises amino acids G85, G86, G87, G88, G89, and E90. In some embodiments, an IL-21 polypeptide or a functional fragment or a variant thereof comprises at least 80% sequence identity to SEQ ID NO: 402 and/or comprises amino acids: G85, G86, G87, G88, G89, and G90. In some embodiments, an IL-21 polypeptide or a functional fragment or a variant thereof comprises at least 85%, 90%, 95%, 96%, 97%, 98%, 99% or 100% sequence identity to SEQ ID NO: 402 and/or comprises amino acids G85, G86, G87, G88, G89, and G90. In some embodiments, an IL-21 polypeptide or a functional fragment or a variant thereof comprises at least 80% sequence identity to SEQ ID NO: 403 and/or comprises amino acids: G85, G86, E87, G88, G89, and G90. In some embodiments, an IL-21 polypeptide or a functional fragment or a variant thereof comprises at least 85%, 90%, 95%, 96%, 97%, 98%, 99% or 100% sequence identity to SEQ ID NO: 403 and/or comprises amino acids G85, G86, E87, G88, G89, and G90. In some embodiments, an IL-21 polypeptide comprises at least 80% sequence identity to SEQ ID NO: 404 and/or comprises amino acids: G80, G81, G82, S83, G85, G86, S87, G88, G89, G90, S91 and G92. In some embodiments, an IL-21 polypeptide comprises at least 85%, 90%, 95%, 96%, 97%, 98%, 99% or 100% sequence identity to SEQ ID NO: 404 and/or comprises amino acids G80, G81, G82, S83, G85, G86, S87, G88, G89, G90, S91 and G92. In some embodiments, an IL-21 polypeptide comprises at least 80% sequence identity to SEQ ID NO: 405 and/or comprises amino acids: G80, G81, G82, G83, G85, G86, G87, G88, G89, G90, G91 and G92. In some embodiments, an IL-21 polypeptide comprises at least 85%, 90%, 95%, 96%, 97%, 98%, 99% or 100% sequence identity to SEQ ID NO: 405 and/or comprises amino acids G80, G81, G82, G83, G85, G86, G87, G88, G89, G90, G91 and G92. In some embodiments, an IL-21 polypeptide comprises at least 80% sequence identity to SEQ ID NO: 406 and/or comprises amino acids: G80, G81, G82, S83, G85, G86, E87, G88, G89, G90, S91 and G92. In some embodiments, an IL-21 polypeptide comprises at least 85%, 90%, 95%, 96%, 97%, 98%, 99% or 100% sequence identity to SEQ ID NO: 406 and/or comprises amino acids G80, G81, G82, S83, G85, G86, E87, G88, G89, G90, S91 and G92. In some embodiments, an IL-21 polypeptide comprises at least 80% sequence identity to SEQ ID NO: 407 and/or comprises amino acids: G80, G81, G82, E83, G85, G86, E87, G88, G89, G90, S91 and G92. In some embodiments, an IL-21 polypeptide comprises at least 85%, 90%, 95%, 96%, 97%, 98%, 99% or 100% sequence identity to SEQ ID NO: 407 and/or comprises amino acids G80, G81, G82, E83, G85, G86, E87, G88, G89, G90, S91 and G92. In some embodiments, an IL-21 polypeptide comprises at least 80% sequence identity to SEQ ID NO: 408 and/or comprises amino acids: G82, G83, S85, G86, G87, G88, S89, G90, G91 and S92. In some embodiments, an IL-21 polypeptide comprises at least 85%, 90%, 95%, 96%, 97%, 98%, 99% or 100% sequence identity to SEQ ID NO: 408 and/or comprises amino acids G82, G83, S85, G86, G87, G88, S89, G90, G91 and S92. In some embodiments, an IL-21 polypeptide comprises at least 80% sequence identity to SEQ ID NO: 409 and/or comprises amino acids: G82, G83, E85, G86, G87, G88, S89, G90, G91 and S92. In some embodiments, an IL-21 polypeptide comprises at least 85%, 90%, 95%, 96%, 97%, 98%, 99% or 100% sequence identity to SEQ ID NO: 409 and/or comprises amino acids G82, G83, E85, G86, G87, G88, S89, G90, G91 and S92. In some embodiments, an IL-21 polypeptide comprises at least 80% sequence identity to SEQ ID NO: 410 and/or comprises amino acids: G80, G81, G82, G83, E85, G86, G87, G88, G89, G90, G91 and G92. In some embodiments, an IL-21 polypeptide comprises at least 85%, 90%, 95%, 96%, 97%, 98%, 99% or 100% sequence identity to SEQ ID NO: 410 and/or comprises amino acids G80, G81, G82, G83, E85, G86, G87, G88, G89, G90, G91 and G92. In some embodiments, an IL-21 polypeptide comprises at least 80% sequence identity to SEQ ID NO: 411 and/or comprises amino acids: G80, G81, G82, S83, G85, G86, S87, G88, G89, E90, G91 and G92. In some embodiments, an IL-21 polypeptide comprises at least 85%, 90%, 95%, 96%, 97%, 98%, 99% or 100% sequence identity to SEQ ID NO: 411 and/or comprises amino acids G80, G81, G82, S83, G85, G86, S87, G88, G89, E90, G91 and G92. In some embodiments, an IL-21 polypeptide comprises at least 80% sequence identity to SEQ ID NO: 412 and/or comprises amino acids: G80, G81, G82, G83, G85, G86, G87, G88, G89, E90, G91 and G92. In some embodiments, an IL-21 polypeptide comprises at least 85%, 90%, 95%, 96%, 97%, 98%, 99% or 100% sequence identity to SEQ ID NO: 412 and/or comprises amino acids G80, G81, G82, G83, G85, G86, G87, G88, G89, E90, G91 and G92. In some embodiments, an IL-21 polypeptide comprises at least 80% sequence identity to SEQ ID NO: 413 and/or comprises amino acids: G82, G83, S85, G86, G87, G88, S89, E90, G91 and S92. In some embodiments, an IL-21 polypeptide comprises at least 85%, 90%, 95%, 96%, 97%, 98%, 99% or 100% sequence identity to SEQ ID NO: 413 and/or comprises amino acids G82, G83, S85, G86, G87, G88, S89, E90, G91 and S92. In some embodiments, an IL-21 polypeptide comprises at least 80% sequence identity to SEQ ID NO: 414 and/or comprises amino acids: G82, G83, G85, G86, G87, G88, G89, E90, G91 and G92. In some embodiments, an IL-21 polypeptide comprises at least 85%, 90%, 95%, 96%, 97%, 98%, 99% or 100% sequence identity to SEQ ID NO: 414 and/or comprises amino acids G82, G83, G85, G86, G87, G88, G89, E90, G91 and G92. In some embodiments, an IL-21 polypeptide comprises at least 80% sequence identity to SEQ ID NO: 415 and/or comprises amino acids: G85, G86, G87, G88, G89, E90, and G91. In some embodiments, an IL-21 polypeptide comprises at least 85%, 90%, 95%, 96%, 97%, 98%, 99% or 100% sequence identity to SEQ ID NO: 415 and/or comprises amino acids G85, G86, G87, G88, G89, E90, and G91. In some embodiments, an IL-21 polypeptide comprises at least 80% sequence identity to SEQ ID NO: 416 and/or comprises amino acids: G82, G83, G85, G86, G87, G88, G89, E90, and G91. In some embodiments, an IL-21 polypeptide comprises at least 85%, 90%, 95%, 96%, 97%, 98%, 99% or 100% sequence identity to SEQ ID NO: 416 and/or comprises amino acids G82, G83, G85, G86, G87, G88, G89, E90, and G91. In some embodiments, an IL-21 polypeptide comprises at least 80% sequence identity to SEQ ID NO: 417 and/or comprises amino acids: G82, G83, G85, E86, G87, G88, G89, E90, and G91. In some embodiments, an IL-21 polypeptide comprises at least 85%, 90%, 95%, 96%, 97%, 98%, 99% or 100% sequence identity to SEQ ID NO: 417 and/or comprises amino acids G82, G83, G85, E86, G87, G88, G89, E90, and G91. In some embodiments, an IL-21 polypeptide comprises at least 80% sequence identity to SEQ ID NO: 418 and/or comprises amino acids: G82, G83, E85, G86, G87, G88, G89, E90, and G91. In some embodiments, an IL-21 polypeptide comprises at least 85%, 90%, 95%, 96%, 97%, 98%, 99% or 100% sequence identity to SEQ ID NO: 418 and/or comprises amino acids G82, G83, E85, G86, G87, G88, G89, E90, and G91. In some embodiments, an IL-21 polypeptide comprises at least 80% sequence identity to SEQ ID NO: 419 and/or comprises amino acids: G80, G81, G82, G83, E84, G85, G86, G87, G88, G89, E90, G91 and G92. In some embodiments, an IL-21 polypeptide comprises at least 85%, 90%, 95%, 96%, 97%, 98%, 99% or 100% sequence identity to SEQ ID NO: 419 and/or comprises amino acids G80, G81, G82, G83, E84, G85, G86, G87, G88, G89, E90, G91 and G92. In some embodiments, an IL-21 polypeptide comprises at least 80% sequence identity to SEQ ID NO: 420 and/or comprises amino acids: G80, G81, G82, E83, G85, G86, G87, G88, G89, E90, G91 and G92. In some embodiments, an IL-21 polypeptide comprises at least 85%, 90%, 95%, 96%, 97%, 98%, 99% or 100% sequence identity to SEQ ID NO: 420 and/or comprises amino acids G80, G81, G82, E83, G85, G86, G87, G88, G89, E90, G91 and G92. In some embodiments, an IL-21 polypeptide comprises at least 80% sequence identity to SEQ ID NO: 421 and/or comprises amino acids: G80, G81, E82, G83, G85, G86, G87, G88, G89, E90, G91 and G92. In some embodiments, an IL-21 polypeptide comprises at least 85%, 90%, 95%, 96%, 97%, 98%, 99% or 100% sequence identity to SEQ ID NO: 421 and/or comprises amino acids G80, G81, E82, G83, G85, G86, G87, G88, G89, E90, G91 and G92.

In some embodiments, the IL-21 polypeptide comprises the amino acid sequence of SEQ ID NO: 391. In some embodiments, the IL-21 polypeptide comprises the amino acid sequence of SEQ ID NO: 392. In some embodiments, the IL-21 polypeptide comprises the amino acid sequence of SEQ ID NO: 393. In some embodiments, the IL-21 polypeptide comprises the amino acid sequence of SEQ ID NO: 394. In some embodiments, the IL-21 polypeptide comprises the amino acid sequence of SEQ ID NO: 395. In some embodiments, the IL-21 polypeptide comprises the amino acid sequence of SEQ ID NO: 396. In some embodiments, the IL-21 polypeptide comprises the amino acid sequence of SEQ ID NO: 397. In some embodiments, the IL-21 polypeptide comprises the amino acid sequence of SEQ ID NO: 398. In some embodiments, the IL-21 polypeptide comprises the amino acid sequence of SEQ ID NO: 399. In some embodiments, the IL-21 polypeptide comprises the amino acid sequence of SEQ ID NO: 400. In some embodiments, the IL-21 polypeptide comprises the amino acid sequence of SEQ ID NO: 401. In some embodiments, the IL-21 polypeptide comprises the amino acid sequence of SEQ ID NO: 402. In some embodiments, the IL-21 polypeptide comprises the amino acid sequence of SEQ ID NO: 403. In some embodiments, the IL-21 polypeptide comprises the amino acid sequence of SEQ ID NO: 404. In some embodiments, the IL-21 polypeptide comprises the amino acid sequence of SEQ ID NO: 405. In some embodiments, the IL-21 polypeptide comprises the amino acid sequence of SEQ ID NO: 406. In some embodiments, the IL-21 polypeptide comprises the amino acid sequence of SEQ ID NO: 407. In some embodiments, the IL-21 polypeptide comprises the amino acid sequence of SEQ ID NO: 408. In some embodiments, the IL-21 polypeptide comprises the amino acid sequence of SEQ ID NO: 409. In some embodiments, the IL-21 polypeptide comprises the amino acid sequence of SEQ ID NO: 410. In some embodiments, the IL-21 polypeptide comprises the amino acid sequence of SEQ ID NO: 411. In some embodiments, the IL-21 polypeptide comprises the amino acid sequence of SEQ ID NO: 412. In some embodiments, the IL-21 polypeptide comprises the amino acid sequence of SEQ ID NO: 413. In some embodiments, the IL-21 polypeptide comprises the amino acid sequence of SEQ ID NO: 414. In some embodiments, the IL-21 polypeptide comprises the amino acid sequence of SEQ ID NO: 415. In some embodiments, the IL-21 polypeptide comprises the amino acid sequence of SEQ ID NO: 416. In some embodiments, the IL-21 polypeptide comprises the amino acid sequence of SEQ ID NO: 417. In some embodiments, the IL-21 polypeptide comprises the amino acid sequence of SEQ ID NO: 418. In some embodiments, the IL-21 polypeptide comprises the amino acid sequence of SEQ ID NO: 419. In some embodiments, the IL-21 polypeptide comprises the amino acid sequence of SEQ ID NO: 420. In some embodiments, the IL-21 polypeptide comprises the amino acid sequence of SEQ ID NO: 421.

In some embodiments, an IL-21 polypeptide comprises QGQDRHMIRMRQLIDIVDQLKNYVNDLVPEFLPAPEDVETNCEWSAFSCFQKAQLXi SANTGNNERIINVSIKKLX₂RKPPX₃X₄X₅X₆X₇X₈X₉X₁₀X₁₁X₁₂X₁₃X₁₄X₁₅CPSCDSYEKKP PKEFLERFKSLLQKMIHQHLSSRTHGSEDS (SEQ ID NO: 553), wherein X₁=A, E, K; X₂=A, E, K; X₃=G, S; X₄=G, T; X₅=G, E, N; X₆=G, S, E, A; X₇=E, G; X₈=G, E, S, R; X₉=G, E, R; X₁₀=S, G, E, Q; X₁₁=G, K; X₁₂=G, S, H; X₁₃=A, E, S, G, R; X₁₄=S, G, L; X₁₅=G, S, T; and wherein at least one amino acid residue is not the amino acid residue at the identical position set forth in SEQ ID NO: 390. In some embodiments, at least one of X₈, X₉, X₁₁, X₁₂, and X₁₃ is not the amino acid residue at the identical position set forth in SEQ ID NO: 390.

In some embodiments, the IL-21 polypeptide comprises the amino acid sequence: QGQDRHMIRMRQLIDIVDQLKNYVNDLVPEFLPAPEDVETNCEWSAFSCFQKAQLK SANTGNNERIINVSIKKLKRKPPX₁X₂X₃X₄GX₅X₆X₇X₈X₉X₁₀X₁₁X₁₂CPSCDSYEKKPPK EFLERFKSLLQKMIHQHLSSRTHGSEDS (SEQ ID NO: 554), wherein X₁=G, S; X₂=G, T; X₃=G, E, N; X₄=G, S, E, A; X₅=G, E, S, R; X₆=G, E, R; X₇=S, G, E, Q; X₈=G, K; X₉=G, S, H; X₁₀=A, E, S, G, R; X₁₁=S, G, L; and X₁₂=G, S, T, provided at least one of X₅, X₆, X₈, X₉, and X₁₀ is not the amino acid residue at the identical position set forth in SEQ ID NO: 390. In some embodiments, X₅=G, X₆=G, X₈=G, X₁₀=A. In some embodiments, X₅=G, X₆=G, X₈=G, X₁₀=E. In some embodiments, X₁=G, X₂=G, X₃=G, X₄=S, X₅=E, X₆=G, X₇=S, X₈=G, X₉=G, X₁₀=S. In some embodiments, X₁=G, X₂=G, X₃=G, X₄=S, X₅=G, X₆=G, X₇=S, X₈=G, X₉=G, X₁₀=E. In some embodiments, X₅=G, X₆=G, X₇=S, X₈=G, X₉=G, X₁₀=E. In some embodiments, X₅=G, X₆=G, X₇=S, X₈=G, X₉=G, X₁₀=S. In some embodiments, X₅=G, X₆=G, X₇=G, X₈=G, X₉=G, X₁₀=E. In some embodiments, X₅=G, X₆=G, X₇=G, X₈=G, X₉=G, X₁₀=G. In some embodiments, X₅=G, X₆=G, X₇=E, X₈=G, X₉=G, X₁₀=G. In some embodiments, X₁=G, X₂=G, X₃=G, X₄=S, X₅=G, X₆=G, X₇=S, X₈=G, X₉=G, X₁₀=G, X₁=S, X₁₂=G. In some embodiments, X₁=G, X₂=G, X₃=G, X₄=G, X₅=G, X₆=G, X₇=G, X₈=G, X₉=G, X₁₀=G, X₁₁=G, X₁₂=G. In some embodiments, X₁=G, X₂=G, X₃=G, X₄=S, X₅=G, X₆=G, X₇=E, X₈=G, X₉=G, X₁₀=G, X₁₁=S, X₁₂=G. In some embodiments, X₁=G, X₂=G, X₃=G, X₄=E, X₅=G, X₆=G, X₇=E, X₈=G, X₉=G, X₁₀=G, X₁=S, X₁₂=G. In some embodiments, X₃=G, X₄=G, X₅=S, X₆=G, X₇=G, X₈=G, X₉=S, X₁₀=G, X₁₁=G, X₁₂=S. In some embodiments, X₃=G, X₄=G, X₅=E, X₆=G, X₇=G, X₈=G, X₉=S, X₁₀=G, X₁₁=G, X₁₂=S. In some embodiments, X₁=G, X₂=G, X₃=G, X₄=G, X₅=E, X₆=G, X₇=G, X₈=G, X₉=G, X₁₀=G, X₁=G, X₁₂=G. In some embodiments, X₁=G, X₂=G, X₃=G, X₄=S, X₅=G, X₆=G, X₇=S, X₈=G, X₉=G, X₁₀=E, X₁₁=G, X₁₂=G. In some embodiments, X₁=G, X₂=G, X₃=G, X₄=G, X₅=G, X₆=G, X₇=G, X₈=G, X₉=G, X₁₀=E, X₁₁=G, X₁₂=G. In some embodiments, X₃=G, X₄=G, X₅=S, X₆=G, X₇=G, X₈=G, X₉=S, X₁₀=E, X₁₁=G, X₁₂=S. In some embodiments, X₃=G, X₄=G, X₅=G, X₆=G, X₇=G, X₈=G, X₉=G, X₁₀=E, X₁₁=G, X₁₂=G. In some embodiments, X₅=G, X₆=G, X₇=G, X₈=G, X₉=G, X₁₀=E, X₁=G. In some embodiments, X₃=G, X₄=G, X₅=G, X₆=G, X₇=G, X₈=G, X₉=G, X₁₀=E, X₁₁=G. In some embodiments, X₃=G, X₄=G, X₅=G, X₆=E, X₇=G, X₈=G, X₉=G, X₁₀=E, X₁₁=G. In some embodiments, X₃=G, X₄=G, X₅=E, X₆=G, X₇=G, X₈=G, X₉=G, X₁₀=E, X₁=G. In some embodiments, X₁=G, X₂=G, X₃=G, X₄=E, X₅=G, X₆=G, X₇=G, X₈=G, X₉=G, X₁₀=E, X₁₁=G, X₁₂=G. In some embodiments, X₁=G, X₂=G, X₃=E, X₄=G, X₅=G, X₆=G, X₇=G, X₈=G, X₉=G, X₁₀=E, X₁₁=G, X₁₂=G.

In some embodiments, the IL-21 polypeptide comprises an amino acid sequence starting at position 78 as defined by SEQ ID NO: 390, wherein the amino acid sequence is PPX₁X₂X₃X₄GX₅X₆X₇X₈X₉X₁₀X₁₁X₁₂CP (SEQ ID NO: 555), wherein X₁=G, S; X₂=G, T; X₃=G, E, N; X₄=G, S, E, A; X₅=G, E, S, R; X₆=G, E, R; X₇=S, G, E, Q; X₈=G, K; X₉=G, S, H; X₁₀=A, E, S, G, R; X₁₁=S, G, L; and X₁₂=G, S, T, provided at least one of X₅, X₆, X₈, X₉, and X₁₀ is not the amino acid residue at the identical position set forth in SEQ ID NO: 390.

In some embodiments, the IL-21 polypeptide comprises an amino acid sequence starting at position 80 as defined by SEQ ID NO: 390, wherein the amino acid sequence is X₁X₂X₃X₄GX₅X₆X₇X₈X₉X₁₀X₁₁X₁₂, wherein X₁=G, S; X₂=G, T; X₃=G, E, N; X₄=G, S, E, A; X₅=G, E, S, R; X₆=G, E, R; X₇=S, G, E, Q; X₈=G, K; X₉=G, S, H; X₁₀=A, E, S, G, R; X₁₁=S, G, L; and X₁₂=G, S, T, provided at least one of X₅, X₆, X₈, X₉, and X₁₀ is not the amino acid residue at the identical position set forth in SEQ ID NO: 390.

Exemplary IL-21 polypeptides comprising at least one amino acid mutation that reduces the isoelectric point are provided in Table 7.

TABLE 7
IL-21 Polypeptides with Reduced Isoelectric Point

| IL-21 | | Theoretical | SEQ |
|---|---|---|---|
| Variant | Amino Acid Sequence | pI | ID NO |
| Exemplary | QGQDRHMIRMRQLIDIVDQLKNYVNDLVPEFLPAPEDVE | 8.72 | 392 |
| IL-21 | TNCEWSAFSCFQKAQLKSANTGNNERIINVSIKKLKRKP | | |
| polypeptide | PSTNAGGGQGHALTCPSCDSYEKKPPKEFLERFKSLLQK | | |
| sequence 1 | MIHQHLSSRTHGSEDS | | |
| Exemplary | QGQDRHMIRMRQLIDIVDQLKNYVNDLVPEFLPAP | 8.39 | 391 |
| IL-21 | EDVETNCEWSAFSCFQKAQLKSANTGNNERIINVS | | |
| polypeptide | IKKLKRKPPSTNAGGGQGHELTCPSCDSYEKKPPK | | |
| sequence 2 | EFLERFKSLLQKMIHQHLSSRTHGSEDS | | |
| Exemplary | QGQDRHMIRMRQLIDIVDQLKNYVNDLVPEFLPAP | 7.87 | 393 |
| IL-21 | EDVETNCEWSAFSCFQKAQLASANTGNNERIINVS | | |
| polypeptide | IKKLARKPPSTNAGGGQGHALTCPSCDSYEKKPPK | | |
| sequence 3 | EFLERFKSLLQKMIHQHLSSRTHGSEDS | | |
| Exemplary | QGQDRHMIRMRQLIDIVDQLKNYVNDLVPEFLPAP | 7.12 | 394 |
| IL-21 | EDVETNCEWSAFSCFQKAQLESANTGNNERIINVS | | |
| polypeptide | IKKLARKPPSTNAGGGQGHALTCPSCDSYEKKPPK | | |
| sequence 4 | EFLERFKSLLQKMIHQHLSSRTHGSEDS | | |
| Exemplary | QGQDRHMIRMRQLIDIVDQLKNYVNDLVPEFLPAP | 7.12 | 395 |
| IL-21 | EDVETNCEWSAFSCFQKAQLASANTGNNERIINVS | | |
| polypeptide | IKKLERKPPSTNAGGGQGHALTCPSCDSYEKKPPK | | |
| sequence 5 | EFLERFKSLLQKMIHQHLSSRTHGSEDS | | |
| Exemplary | QGQDRHMIRMRQLIDIVDQLKNYVNDLVPEFLPAP | 7.12 | 396 |
| IL-21 | EDVETNCEWSAFSCFQKAQLASANTGNNERIINVS | | |
| polypeptide | IKKLARKPPSTNAGGGQGHELTCPSCDSYEKKPPK | | |
| sequence 6 | EFLERFKSLLQKMIHQHLSSRTHGSEDS | | |
| Exemplary | QGQDRHMIRMRQLIDIVDQLKNYVNDLVPEFLPAP | 8.39 | 397 |
| IL-21 | EDVETNCEWSAFSCFQKAQLKSANTGNNERIINVS | | |
| polypeptide | IKKLKRKPPGGGSGEGSGGSLTCPSCDSYEKKPPK | | |
| sequence 7 | EFLERFKSLLQKMIHQHLSSRTHGSEDS | | |
| Exemplary | QGQDRHMIRMRQLIDIVDQLKNYVNDLVPEFLPAP | 8.39 | 398 |
| IL-21 | EDVETNCEWSAFSCFQKAQLKSANTGNNERIINVS | | |
| polypeptide | IKKLKRKPPGGGSGGGSGGELTCPSCDSYEKKPPK | | |
| sequence 8 | EFLERFKSLLQKMIHQHLSSRTHGSEDS | | |
| Exemplary | QGQDRHMIRMRQLIDIVDQLKNYVNDLVPEFLPAP | 8.39 | 399 |
| IL-21 | EDVETNCEWSAFSCFQKAQLKSANTGNNERIINVS | | |
| polypeptide | IKKLKRKPPSTNAGGGSGGELTCPSCDSYEKKPPK | | |
| sequence 9 | EFLERFKSLLQKMIHQHLSSRTHGSEDS | | |
| Exemplary | QGQDRHMIRMRQLIDIVDQLKNYVNDLVPEFLPAP | 8.72 | 400 |
| IL-21 | EDVETNCEWSAFSCFQKAQLKSANTGNNERIINVS | | |
| polypeptide | IKKLKRKPPSTNAGGGSGGSLTCPSCDSYEKKPPK | | |
| sequence 10 | EFLERFKSLLQKMIHQHLSSRTHGSEDS | | |
| Exemplary | QGQDRHMIRMRQLIDIVDQLKNYVNDLVPEFLPAP | 8.39 | 401 |
| IL-21 | EDVETNCEWSAFSCFQKAQLKSANTGNNERIINVS | | |
| polypeptide | IKKLKRKPPSTNAGGGGGGELTCPSCDSYEKKPPK | | |
| sequence 11 | EFLERFKSLLQKMIHQHLSSRTHGSEDS | | |
| Exemplary | QGQDRHMIRMRQLIDIVDQLKNYVNDLVPEFLPAP | 8.72 | 402 |
| IL-21 | EDVETNCEWSAFSCFQKAQLKSANTGNNERIINVS | | |
| polypeptide | IKKLKRKPPSTNAGGGGGGGLTCPSCDSYEKKPPK | | |
| sequence 12 | EFLERFKSLLQKMIHQHLSSRTHGSEDS | | |
| Exemplary | QGQDRHMIRMRQLIDIVDQLKNYVNDLVPEFLPAP | 8.39 | 403 |
| IL-21 | EDVETNCEWSAFSCFQKAQLKSANTGNNERIINVS | | |
| polypeptide | IKKLKRKPPSTNAGGGEGGGLTCPSCDSYEKKPPK | | |
| sequence 13 | EFLERFKSLLQKMIHQHLSSRTHGSEDS | | |
| Exemplary | QGQDRHMIRMRQLIDIVDQLKNYVNDLVPEFLPAP | 8.72 | 404 |
| IL-21 | EDVETNCEWSAFSCFQKAQLKSANTGNNERIINVS | | |
| polypeptide | IKKLKRKPPGGGSGGGSGGGSGCPSCDSYEKKPPK | | |
| sequence 14 | EFLERFKSLLQKMIHQHLSSRTHGSEDS | | |
| Exemplary | QGQDRHMIRMRQLIDIVDQLKNYVNDLVPEFLPAP | 8.72 | 405 |
| IL-21 | EDVETNCEWSAFSCFQKAQLKSANTGNNERIINVS | | |
| polypeptide | IKKLKRKPPGGGGGGGGGGGGGCPSCDSYEKKPPK | | |
| sequence 15 | EFLERFKSLLQKMIHQHLSSRTHGSEDS | | |
| Exemplary | QGQDRHMIRMRQLIDIVDQLKNYVNDLVPEFLPAP | 8.39 | 406 |
| IL-21 | EDVETNCEWSAFSCFQKAQLKSANTGNNERIINVS | | |
| polypeptide | IKKLKRKPPGGGSGGGEGGGSGCPSCDSYEKKPPK | | |
| sequence 16 | EFLERFKSLLQKMIHQHLSSRTHGSEDS | | |
| Exemplary | QGQDRHMIRMRQLIDIVDQLKNYVNDLVPEFLPAP | 7.85 | 407 |
| IL-21 | EDVETNCEWSAFSCFQKAQLKSANTGNNERIINVS | | |
| polypeptide | IKKLKRKPPGGGEGGGEGGGSGCPSCDSYEKKPPK | | |
| sequence 17 | EFLERFKSLLQKMIHQHLSSRTHGSEDS | | |
| Exemplary | QGQDRHMIRMRQLIDIVDQLKNYVNDLVPEFLPAP | 8.72 | 408 |
| IL-21 | EDVETNCEWSAFSCFQKAQLKSANTGNNERIINVS | | |
| polypeptide | IKKLKRKPPSTGGGSGGGSGGSCPSCDSYEKKPPK | | |
| sequence 18 | EFLERFKSLLQKMIHQHLSSRTHGSEDS | | |
| Exemplary | QGQDRHMIRMRQLIDIVDQLKNYVNDLVPEFLPAP | 8.39 | 409 |
| IL-21 | EDVETNCEWSAFSCFQKAQLKSANTGNNERIINVS | | |
| polypeptide | IKKLKRKPPSTGGGEGGGSGGSCPSCDSYEKKPPK | | |
| sequence 19 | EFLERFKSLLQKMIHQHLSSRTHGSEDS | | |
| Exemplary | QGQDRHMIRMRQLIDIVDQLKNYVNDLVPEFLPAP | 8.39 | 410 |
| IL-21 | EDVETNCEWSAFSCFQKAQLKSANTGNNERIINVS | | |
| polypeptide | IKKLKRKPPGGGGGEGGGGGGGCPSCDSYEKKPPK | | |
| sequence 20 | EFLERFKSLLQKMIHQHLSSRTHGSEDS | | |
| Exemplary | QGQDRHMIRMRQLIDIVDQLKNYVNDLVPEFLPAP | 8.39 | 411 |
| IL-21 | EDVETNCEWSAFSCFQKAQLKSANTGNNERIINVS | | |
| polypeptide | IKKLKRKPPGGGSGGGSGGEGGCPSCDSYEKKPPK | | |
| sequence 21 | EFLERFKSLLQKMIHQHLSSRTHGSEDS | | |
| Exemplary | QGQDRHMIRMRQLIDIVDQLKNYVNDLVPEFLPAP | 8.39 | 412 |
| IL-21 | EDVETNCEWSAFSCFQKAQLKSANTGNNERIINVS | | |
| polypeptide | IKKLKRKPPGGGGGGGGGGEGGCPSCDSYEKKPPK | | |
| sequence 22 | EFLERFKSLLQKMIHQHLSSRTHGSEDS | | |
| Exemplary | QGQDRHMIRMRQLIDIVDQLKNYVNDLVPEFLPAP | 8.39 | 413 |
| IL-21 | EDVETNCEWSAFSCFQKAQLKSANTGNNERIINVS | | |
| polypeptide | IKKLKRKPPSTGGGSGGGSEGSCPSCDSYEKKPPK | | |
| sequence 23 | EFLERFKSLLQKMIHQHLSSRTHGSEDS | | |
| Exemplary | QGQDRHMIRMRQLIDIVDQLKNYVNDLVPEFLPAP | 8.39 | 414 |
| IL-21 | EDVETNCEWSAFSCFQKAQLKSANTGNNERIINVS | | |
| polypeptide | IKKLKRKPPSTGGGGGGGGEGGCPSCDSYEKKPPK | | |
| sequence 24 | EFLERFKSLLQKMIHQHLSSRTHGSEDS | | |
| Exemplary | QGQDRHMIRMRQLIDIVDQLKNYVNDLVPEFLPAP | 8.39 | 415 |
| IL-21 | EDVETNCEWSAFSCFQKAQLKSANTGNNERIINVS | | |
| polypeptide | IKKLKRKPPSTNAGGGGGGEGTCPSCDSYEKKPPK | | |
| sequence 25 | EFLERFKSLLQKMIHQHLSSRTHGSEDS | | |
| Exemplary | QGQDRHMIRMRQLIDIVDQLKNYVNDLVPEFLPAP | 8.39 | 416 |
| IL-21 | EDVETNCEWSAFSCFQKAQLKSANTGNNERIINVS | | |
| polypeptide | IKKLKRKPPSTGGGGGGGGEGTCPSCDSYEKKPPK | | |
| sequence 26 | EFLERFKSLLQKMIHQHLSSRTHGSEDS | | |
| Exemplary | QGQDRHMIRMRQLIDIVDQLKNYVNDLVPEFLPAP | 7.85 | 417 |
| IL-21 | EDVETNCEWSAFSCFQKAQLKSANTGNNERIINVS | | |
| polypeptide | IKKLKRKPPSTGGGGEGGGEGTCPSCDSYEKKPPK | | |
| sequence 27 | EFLERFKSLLQKMIHQHLSSRTHGSEDS | | |
| Exemplary | QGQDRHMIRMRQLIDIVDQLKNYVNDLVPEFLPAP | 7.85 | 418 |
| IL-21 | EDVETNCEWSAFSCFQKAQLKSANTGNNERIINVS | | |
| polypeptide | IKKLKRKPPSTGGGEGGGGEGTCPSCDSYEKKPPK | | |
| sequence 28 | EFLERFKSLLQKMIHQHLSSRTHGSEDS | | |
| Exemplary | QGQDRHMIRMRQLIDIVDQLKNYVNDLVPEFLPAP | 7.85 | 419 |
| IL-21 | EDVETNCEWSAFSCFQKAQLKSANTGNNERIINVS | | |
| polypeptide | IKKLKRKPPGGGGEGGGGGEGGCPSCDSYEKKPPK | | |
| sequence 29 | EFLERFKSLLQKMIHQHLSSRTHGSEDS | | |
| Exemplary | QGQDRHMIRMRQLIDIVDQLKNYVNDLVPEFLPAP | 7.85 | 420 |
| IL-21 | EDVETNCEWSAFSCFQKAQLKSANTGNNERIINVS | | |
| polypeptide | IKKLKRKPPGGGEGGGGGGEGGCPSCDSYEKKPPK | | |
| sequence 30 | EFLERFKSLLQKMIHQHLSSRTHGSEDS | | |
| Exemplary | QGQDRHMIRMRQLIDIVDQLKNYVNDLVPEFLPAP | 7.85 | 421 |
| IL-21 | EDVETNCEWSAFSCFQKAQLKSANTGNNERIINVS | | |
| polypeptide | IKKLKRKPPGGEGGGGGGGEGGCPSCDSYEKKPPK | | |
| sequence 31 | EFLERFKSLLQKMIHQHLSSRTHGSEDS | | |

### Reduced IL-21R Binding

In some embodiments, the disclosure provides an IL-21 polypeptide with reduced binding affinity to IL-21R. In some embodiments, an IL-21 comprises at least one mutation that reduces its binding affinity to IL-21R. Such mutations are in some embodiments in one or more positions selected from the group consisting of: R5, I8, R9, R11, Q12, 114, D15, D18, Q19, Y23, R65, S70, K72, K73, K75, R76, K77, S80, Q116, and K117, wherein the position numbering is number according to the amino acid sequence of SEQ ID NO: 390. In some embodiments, such mutations are in one or more positions selected from the group consisting of: R5, I8, R9, R11, L13, I14, I16, V17, D18, K72, K73, L74, K75, R76, K77, and K117, wherein the position numbering is number according to the amino acid sequence of SEQ ID NO: 390. In some embodiments, the mutation is at position R5. In some embodiments, the mutation is at position 18. In some embodiments, the mutation is at position R9. In some embodiments, the mutation is at position R11. In some embodiments, the mutation is at position Q12. In some embodiments, the mutation is at position 114. In some embodiments, the mutation is at position D15. In some embodiments, the mutation is at position D18. In some embodiments, the mutation is at position Q19. In some embodiments, the mutation is at position Y23. In some embodiments, the mutation is at position R65. In some embodiments, the mutation is at position S70. In some embodiments, the mutation is at position K72. In some embodiments, the mutation is at position K73. In some embodiments, the mutation is at position K75. In some embodiments, the mutation is at position R76. In some embodiments, the mutation is at position K77. In some embodiments, the mutation is at position S80. In some embodiments, the mutation is at position Q116. In some embodiments, the mutation is at position K117. In some embodiments, the mutation at position R5 comprises an amino acid substitution selected from the group consisting of A, D, E, S, T, N, Q, V, I, L, Y, or F. In some embodiments, the mutation at position 18 comprises an amino acid substitution selected from the group consisting of Q, H, E. In some embodiments, the mutation at position R9 comprises an amino acid substitution selected from the group consisting of A, D, E, S, T, N, Q, V, I, L, Y, or F. In some embodiments, the mutation at position R11 comprises an amino acid substitution selected from the group consisting of D or E. In some embodiments, the mutation at position Q12 comprises an amino acid substitution selected from the group consisting of L, I, or Y. In some embodiments, the mutation at position L13 comprises an amino acid substitution selected from the group consisting of F or R. In some embodiments, the mutation at position 114 comprises an amino acid substitution selected from the group consisting of D or E. In some embodiments, the mutation at position D15 comprises an amino acid substitution selected from the group consisting of R, K, H, L, Y, or F. In some embodiments, the mutation at position 116 comprises an amino acid substitution selected from the group consisting of A, S or R. In some embodiments, the mutation at position V17 comprises an amino acid substitution selected from the group consisting of I or A. In some embodiments, the mutation at position D18 comprises an amino acid substitution selected from the group consisting of A, K, or R. In some embodiments, the mutation at position Q19 comprises an amino acid substitution selected from the group consisting of L, or Y. In some embodiments, the mutation at position Y23 comprises an amino acid substitution of E. In some embodiments, the mutation at position R65 comprises an amino acid substitution selected from the group consisting of G, S, E, D or A. In some embodiments, the mutation at position S70 comprises an amino acid substitution selected from the group consisting of H, Y, L, V or F. In some embodiments, the mutation at position K72 comprises an amino acid substitution selected from the group consisting of G, S, E, D or A. In some embodiments, the mutation at position K73 comprises an amino acid substitution selected from the group consisting of A, Y, L, F, G, S, T, E, or D. In some embodiments, the mutation at position L74 comprises an amino acid substitution selected from the group consisting of I, F, V, or M. In some embodiments, the mutation at position K75 comprises an amino acid substitution selected from the group consisting of G, S, E, D or A. In some embodiments, the mutation at position R76 comprises an amino acid substitution selected from the group consisting of A, D, E, S, T, N, Q, V, I, L, Y, or F. In some embodiments, the mutation at position K77 comprises an amino acid substitution selected from the group consisting of G, S, E, D or A. In some embodiments, the mutation at position S80 comprises an amino acid substitution of H, A, G, E, or D. In some embodiments, the mutation at position Q116 comprises an amino acid substitution is Y. In some embodiments, the mutation at position K117 comprises an amino acid substitution selected from the group consisting of A, D, or E.

An example sequence for an IL-21 polypeptide or a functional fragment or a variant thereof of this disclosure is provided as follows: QGQDX₁HMX₂X₃MX₄X₅LX₆X₇IVX₈X₉LKNX₁₀VNDLVPEFLPAPEDVETNCEWSAFSCF QKAQLKSANTGNNEX₁₁INVX₁₂IX₁₃X₁₄LX₁₅X₁₆X₁₇PPX₁₈TNAGRRQKHRLTCPSCDSY EKKPPKEFLERFKSLLX₁₉X₂₀MIHQHLSSRTHGSEDS (SEQ ID NO: 552). In some embodiments, X₁=R, A, D, E, S, T, N, Q, V, I, L, Y, or F. In some embodiments, X₂=I, Q, H, E. In some embodiments, X₃=R, A, D, E, S, T, N, Q, V, I, L, Y, or F. In some embodiments, X₄=R, D or E. In some embodiments, X₅=Q, L, I, or Y. In some embodiments, X₆=I, D or E. In some embodiments, X₇=D, R, K, H, L, Y, or F. In some embodiments, X₈=D, A, K, or R. In some embodiments, X₉=Q, L, or Y. In some embodiments, X₁₀=Y or E. In some embodiments, X₁₁=R, G, S, E, D, or A. In some embodiments, X₁₂=S, H, Y, L, V, or F. In some embodiments, X₁₃=K, G, S, E, D, or A. In some embodiments, X₁₄=K, A, Y, L, F, G, S, T, E, A, or D. In some embodiments, X₁₅=K, G, S, E, D, or A. In some embodiments, X₁₆=R, A, D, E, S, T, N, Q, V, I, L, Y, or F. In some embodiments, X₁₇=K, G, S, E, D, or A. In some embodiments, X₁₈=S, H, A, G, E, or D. In some embodiments, X₁₉=Q or Y. In some embodiments, X₂₀=K, A, D, or E.

In some embodiments, an IL-21 polypeptide or a functional fragment or a variant thereof with reduced binding to IL-21R comprises the sequence: QGQDX₁HMX₂X₃MX₄QX₅X₆DX₇X₈X₉QLKNYVNDLVPEFLPAPEDVETNCEWSAFSCF QKAQLKSANTGNNERIINVSIX₁₀X₁₁X₁₂X₁₃X₁₄X₁₅PPSTNAGRRQKHRLTCPSCDSYEK KPPKEFLERFKSLLQ X₁₆MIHQHLSSRTHGSEDS (SEQ ID NO: 551), wherein X₁=F, A, E, S, T, N, Q, V, I, L, Y, R; X₂=E, I; X₃=A, D, E, H, S, T, N, G, V, I, L, Y, R; X₄=D, E, R; X₅=F, R, L; X₆=D, I; X₇=A, S, R, I; X₈=I, A, V; X₉=A, D; X₁₀=A, E, K; X₁₁=A, E, K; X₁₂=I, F, M, L; X₁₃=A, K, E; X₁₄=E, F, A, N, D, S, T, Q, V, I, L, Y, M, R; X₁₅=A, E, K; X₁₆=A, K; provided at least one of X₁-X₁₆ is not the amino acid residue at the identical position set forth in SEQ ID NO: 390.

Exemplary IL-21 polypeptides comprising at least one amino acid mutation that reduces affinity to a human IL-21 receptor are provided in Table 8.

In some embodiments, the disclosure provides an IL-21 polypeptide comprising an amino acid sequence at least 80%, 85%, 90%, 91%, 92%, 93%, 94%, 95%, 96%, 97%, 98% or 99% identical to an amino acid sequence selected from SEQ ID NOs: 492-550. In some embodiments, the disclosure provides an IL-21 polypeptide comprising an amino acid sequence selected from SEQ ID NOs: 492-550.

TABLE 8
IL-21 Polypeptides with Reduced Affinity

| | | K_{D} at | SEQ |
|---|---|---|---|
| IL-21 | | 25° C. | ID |
| Mutein | Amino Acid Sequence | (nM) | NO |
| IL-21 | QGQDRHMIRMRQLIDIVDQLKNYVNDLVPEFLPAPEDVETNCEWSA | 0.25 | 390 |
| | FSCFQKAQLKSANTGNNERIINVSIKKLKRKPPSTNAGRRQKHRLT | | |
| | CPSCDSYEKKPPKEFLERFKSLLQKMIHQHLSSRTHGSEDS | | |
| IL-21 | QGQDRHMIRMDQLIDIVDQLKNYVNDLVPEFLPAPEDVETNCEWSA | 0.54 | 492 |
| v0.1 | FSCFQKAQLKSANTGNNERIINVSIKKLKRKPPSTNAGRRQKHRLT | | |
| | CPSCDSYEKKPPKEFLERFKSLLQKMIHQHLSSRTHGSEDS | | |
| IL-21 | QGQDRHMIRMEQLIDIVDQLKNYVNDLVPEFLPAPEDVETNC | 0.37 | 493 |
| v0.2 | EWSAFSCFQKAQLKSANTGNNERIINVSIKKLKRKPPSTNAG | | |
| | RRQKHRLTCPSCDSYEKKPPKEFLERFKSLLQKMIHQHLSSR | | |
| | THGSEDS | | |
| IL-21 | QGQDRHMIRMRQLDDIVAQLKNYVNDLVPEFLPAPEDVETNC | NB | 494 |
| v0.3 | EWSAFSCFQKAQLKSANTGNNERIINVSIKKLKRKPPSTNAG | | |
| | RRQKHRLTCPSCDSYEKKPPKEFLERFKSLLQAMIHQHLSSR | | |
| | THGSEDS | | |
| IL-21 | QGQDRHMIRMRQLIDIVDQLKNYVNDLVPEFLPAPEDVETNC | >10000 | 495 |
| v0.4 | EWSAFSCFQKAQLKSANTGNNERIINVSIKKLKEKPPSTNAG | | |
| | RRQKHRLTCPSCDSYEKKPPKEFLERFKSLLQKMIHQHLSSR | | |
| | THGSEDS | | |
| IL-21 | QGQDFHMIRMRQLIDIVDQLKNYVNDLVPEFLPAPEDVETNC | 5.5 | 496 |
| v0.5 | EWSAFSCFQKAQLKSANTGNNERIINVSIKKLKRKPPSTNAG | | |
| | RRQKHRLTCPSCDSYEKKPPKEFLERFKSLLQKMIHQHLSSR | | |
| | THGSEDS | | |
| IL-21 | QGQDRHMIRMRQLIDIVDQLKNYVNDLVPEFLPAPEDVETNC | 2205 | 497 |
| v0.6 | EWSAFSCFQKAQLKSANTGNNERIINVSIKKLKFKPPSTNAG | | |
| | RRQKHRLTCPSCDSYEKKPPKEFLERFKSLLQKMIHQHLSSR | | |
| | THGSEDS | | |
| IL-21 | QGQDRHMERMRQLIDIVDQLKNYVNDLVPEFLPAPEDVETNC | 171.9 | 498 |
| v0.7 | EWSAFSCFQKAQLKSANTGNNERIINVSIKKLKRKPPSTNAG | | |
| | RRQKHRLTCPSCDSYEKKPPKEFLERFKSLLQKMIHQHLSSR | | |
| | THGSEDS | | |
| IL-21 | QGQDAHMIRMRQLIDIVDQLKNYVNDLVPEFLPAPEDVETNC | 12.4 | 499 |
| v0.8 | EWSAFSCFQKAQLKSANTGNNERIINVSIKKLKRKPPSTNAG | | |
| | RRQKHRLTCPSCDSYEKKPPKEFLERFKSLLQKMIHQHLSSR | | |
| | THGSEDS | | |
| IL-21 | QGQDEHMIRMRQLIDIVDQLKNYVNDLVPEFLPAPEDVETNC | 184.6 | 500 |
| v0.9 | EWSAFSCFQKAQLKSANTGNNERIINVSIKKLKRKPPSTNAG | | |
| | RRQKHRLTCPSCDSYEKKPPKEFLERFKSLLQKMIHQHLSSR | | |
| | THGSEDS | | |
| IL-21 | QGQDSHMIRMRQLIDIVDQLKNYVNDLVPEFLPAPEDVETNC | 20.8 | 501 |
| v0.10 | EWSAFSCFQKAQLKSANTGNNERIINVSIKKLKRKPPSTNAG | | |
| | RRQKHRLTCPSCDSYEKKPPKEFLERFKSLLQKMIHQHLSSR | | |
| | THGSEDS | | |
| IL-21 | QGQDTHMIRMRQLIDIVDQLKNYVNDLVPEFLPAPEDVETNC | 6.6 | 502 |
| v0.11 | EWSAFSCFQKAQLKSANTGNNERIINVSIKKLKRKPPSTNAG | | |
| | RRQKHRLTCPSCDSYEKKPPKEFLERFKSLLQKMIHQHLSSR | | |
| | THGSEDS | | |
| IL-21 | QGQDNHMIRMRQLIDIVDQLKNYVNDLVPEFLPAPEDVETNC | 23.4 | 503 |
| v0.12 | EWSAFSCFQKAQLKSANTGNNERIINVSIKKLKRKPPSTNAG | | |
| | RRQKHRLTCPSCDSYEKKPPKEFLERFKSLLQKMIHQHLSSR | | |
| | THGSEDS | | |
| IL-21 | QGQDQHMIRMRQLIDIVDQLKNYVNDLVPEFLPAPEDVETNC | 7.7 | 504 |
| v0.13 | EWSAFSCFQKAQLKSANTGNNERIINVSIKKLKRKPPSTNAG | | |
| | RRQKHRLTCPSCDSYEKKPPKEFLERFKSLLQKMIHQHLSSR | | |
| | THGSEDS | | |
| IL-21 | QGQDVHMIRMRQLIDIVDQLKNYVNDLVPEFLPAPEDVETNC | 4.6 | 505 |
| v0.14 | EWSAFSCFQKAQLKSANTGNNERIINVSIKKLKRKPPSTNAG | | |
| | RRQKHRLTCPSCDSYEKKPPKEFLERFKSLLQKMIHQHLSSR | | |
| | THGSEDS | | |
| IL-21 | QGQDIHMIRMRQLIDIVDQLKNYVNDLVPEFLPAPEDVETNC | 6.2 | 506 |
| v0.15 | EWSAFSCFQKAQLKSANTGNNERIINVSIKKLKRKPPSTNAG | | |
| | RRQKHRLTCPSCDSYEKKPPKEFLERFKSLLQKMIHQHLSSR | | |
| | THGSEDS | | |
| IL-21 | QGQDLHMIRMRQLIDIVDQLKNYVNDLVPEFLPAPEDVETNC | 2.5 | 507 |
| v0.16 | EWSAFSCFQKAQLKSANTGNNERIINVSIKKLKRKPPSTNAG | | |
| | RRQKHRLTCPSCDSYEKKPPKEFLERFKSLLQKMIHQHLSSR | | |
| | THGSEDS | | |
| IL-21 | QGQDYHMIRMRQLIDIVDQLKNYVNDLVPEFLPAPEDVETNC | 7.2 | 508 |
| v0.17 | EWSAFSCFQKAQLKSANTGNNERIINVSIKKLKRKPPSTNAG | | |
| | RRQKHRLTCPSCDSYEKKPPKEFLERFKSLLQKMIHQHLSSR | | |
| | THGSEDS | | |
| IL-21 | QGQDRHMIRMRQLIDIVDQLKNYVNDLVPEFLPAPEDVETNC | 2976 | 509 |
| v0.18 | EWSAFSCFQKAQLKSANTGNNERIINVSIKKLKAKPPSTNAG | | |
| | RRQKHRLTCPSCDSYEKKPPKEFLERFKSLLQKMIHQHLSSR | | |
| | THGSEDS | | |
| IL-21 | QGQDRHMIRMRQLIDIVDQLKNYVNDLVPEFLPAPEDVETNC | 3981 | 510 |
| v0.19 | EWSAFSCFQKAQLKSANTGNNERIINVSIKKLKNKPPSTNAG | | |
| | RRQKHRLTCPSCDSYEKKPPKEFLERFKSLLQKMIHQHLSSR | | |
| | THGSEDS | | |
| IL-21 | QGQDRHMIRMRQLIDIVDQLKNYVNDLVPEFLPAPEDVETNC | 10000 | 511 |
| v0.20 | EWSAFSCFQKAQLKSANTGNNERIINVSIKKLKDKPPSTNAG | | |
| | RRQKHRLTCPSCDSYEKKPPKEFLERFKSLLQKMIHQHLSSR | | |
| | THGSEDS | | |
| IL-21 | QGQDRHMIRMRQLIDIVDQLKNYVNDLVPEFLPAPEDVETNC | 3671 | 512 |
| v0.21 | EWSAFSCFQKAQLKSANTGNNERIINVSIKKLKSKPPSTNAG | | |
| | RRQKHRLTCPSCDSYEKKPPKEFLERFKSLLQKMIHQHLSSR | | |
| | THGSEDS | | |
| IL-21 | QGQDRHMIRMRQLIDIVDQLKNYVNDLVPEFLPAPEDVETNC | 4085 | 513 |
| v0.22 | EWSAFSCFQKAQLKSANTGNNERIINVSIKKLKTKPPSTNAG | | |
| | RRQKHRLTCPSCDSYEKKPPKEFLERFKSLLQKMIHQHLSSR | | |
| | THGSEDS | | |
| IL-21 | QGQDRHMIRMRQLIDIVDQLKNYVNDLVPEFLPAPEDVETNC | 3375 | 514 |
| v0.23 | EWSAFSCFQKAQLKSANTGNNERIINVSIKKLKQKPPSTNAG | | |
| | RRQKHRLTCPSCDSYEKKPPKEFLERFKSLLQKMIHQHLSSR | | |
| | THGSEDS | | |
| IL-21 | QGQDRHMIRMRQLIDIVDQLKNYVNDLVPEFLPAPEDVETNC | 4351 | 515 |
| v0.24 | EWSAFSCFQKAQLKSANTGNNERIINVSIKKLKVKPPSTNAG | | |
| | RRQKHRLTCPSCDSYEKKPPKEFLERFKSLLQKMIHQHLSSR | | |
| | THGSEDS | | |
| IL-21 | QGQDRHMIRMRQLIDIVDQLKNYVNDLVPEFLPAPEDVETNC | 3639 | 516 |
| v0.25 | EWSAFSCFQKAQLKSANTGNNERIINVSIKKLKIKPPSTNAG | | |
| | RRQKHRLTCPSCDSYEKKPPKEFLERFKSLLQKMIHQHLSSR | | |
| | THGSEDS | | |
| IL-21 | QGQDRHMIRMRQLIDIVDQLKNYVNDLVPEFLPAPEDVETNC | 934.1 | 517 |
| v0.26 | EWSAFSCFQKAQLKSANTGNNERIINVSIKKLKLKPPSTNAG | | |
| | RRQKHRLTCPSCDSYEKKPPKEFLERFKSLLQKMIHQHLSSR | | |
| | THGSEDS | | |
| IL-21 | QGQDRHMIRMRQLIDIVDQLKNYVNDLVPEFLPAPEDVETNC | 1353 | 518 |
| v0.27 | EWSAFSCFQKAQLKSANTGNNERIINVSIKKLKYKPPSTNAG | | |
| | RRQKHRLTCPSCDSYEKKPPKEFLERFKSLLQKMIHQHLSSR | | |
| | THGSEDS | | |
| IL-21 | QGQDRHMIRMRQLIDIVDQLKNYVNDLVPEFLPAPEDVETNC | 0.77 | 519 |
| v0.28 | EWSAFSCFQKAQLKSANTGNNERIINVSIKKLKRAPPSTNAG | | |
| | RRQKHRLTCPSCDSYEKKPPKEFLERFKSLLQKMIHQHLSSR | | |
| | THGSEDS | | |
| IL-21 | QGQDRHMIRMRQLIDIVDQLKNYVNDLVPEFLPAPEDVETNC | 0.69 | 520 |
| v0.29 | EWSAFSCFQKAQLKSANTGNNERIINVSIKKLKREPPSTNAG | | |
| | RRQKHRLTCPSCDSYEKKPPKEFLERFKSLLQKMIHQHLSSR | | |
| | THGSEDS | | |
| IL-21 | QGQDRHMIRMRQLIDIVDQLKNYVNDLVPEFLPAPEDVETNC | 0.72 | 521 |
| v0.30 | EWSAFSCFQKAQLKSANTGNNERIINVSIAKLKRKPPSTNAG | | |
| | RRQKHRLTCPSCDSYEKKPPKEFLERFKSLLQKMIHQHLSSR | | |
| | THGSEDS | | |
| IL-21 | QGQDRHMIRMRQLIDIVDQLKNYVNDLVPEFLPAPEDVETNC | 2.3 | 522 |
| v0.31 | EWSAFSCFQKAQLKSANTGNNERIINVSIEKLKRKPPSTNAG | | |
| | RRQKHRLTCPSCDSYEKKPPKEFLERFKSLLQKMIHQHLSSR | | |
| | THGSEDS | | |
| IL-21 | QGQDRHMIRMRQLIDIVDQLKNYVNDLVPEFLPAPEDVETNC | 0.78 | 523 |
| v0.32 | EWSAFSCFQKAQLKSANTGNNERIINVSIKKLARKPPSTNAG | | |
| | RRQKHRLTCPSCDSYEKKPPKEFLERFKSLLQKMIHQHLSSR | | |
| | THGSEDS | | |
| IL-21 | QGQDRHMIRMRQLIDIVDQLKNYVNDLVPEFLPAPEDVETNC | 1.2 | 524 |
| v0.33 | EWSAFSCFQKAQLKSANTGNNERIINVSIKKLERKPPSTNAG | | |
| | RRQKHRLTCPSCDSYEKKPPKEFLERFKSLLQKMIHQHLSSR | | |
| | THGSEDS | | |
| IL-21 | QGQDRHMIRMRQLIDIVDQLKNYVNDLVPEFLPAPEDVETNC | 1.81 | 525 |
| v0.34 | EWSAFSCFQKAQLKSANTGNNERIINVSIKALKRKPPSTNAG | | |
| | RRQKHRLTCPSCDSYEKKPPKEFLERFKSLLQKMIHQHLSSR | | |
| | THGSEDS | | |
| IL-21 | QGQDRHMIRMRQLIDIVDQLKNYVNDLVPEFLPAPEDVETNC | 511.4 | 526 |
| v0.35 | EWSAFSCFQKAQLKSANTGNNERIINVSIKELKRKPPSTNAG | | |
| | RRQKHRLTCPSCDSYEKKPPKEFLERFKSLLQKMIHQHLSSR | | |
| | THGSEDS | | |
| IL-21 | QGQDFHMIRMRQLIDIVDQLKNYVNDLVPEFLPAPEDVETNC | 40.2 | 527 |
| v0.36 | EWSAFSCFQKAQLKSANTGNNERIINVSIKKLKRAPPSTNAG | | |
| | RRQKHRLTCPSCDSYEKKPPKEFLERFKSLLQKMIHQHLSSR | | |
| | THGSEDS | | |
| IL-21 | QGQDFHMIRMRQLIDIVDQLKNYVNDLVPEFLPAPEDVETNC | 46.4 | 528 |
| v0.37 | EWSAFSCFQKAQLKSANTGNNERIINVSIKKLKREPPSTNAG | | |
| | RRQKHRLTCPSCDSYEKKPPKEFLERFKSLLQKMIHQHLSSR | | |
| | THGSEDS | | |
| IL-21 | QGQDFHMIRMRQLIDIVDQLKNYVNDLVPEFLPAPEDVETNC | 35.0 | 529 |
| v0.38 | EWSAFSCFQKAQLKSANTGNNERIINVSIAKLKRKPPSTNAG | | |
| | RRQKHRLTCPSCDSYEKKPPKEFLERFKSLLQKMIHQHLSSR | | |
| | THGSEDS | | |
| IL-21 | QGQDFHMIRMRQLIDIVDQLKNYVNDLVPEFLPAPEDVETNC | 265.1 | 530 |
| v0.39 | EWSAFSCFQKAQLKSANTGNNERIINVSIEKLKRKPPSTNAG | | |
| | RRQKHRLTCPSCDSYEKKPPKEFLERFKSLLQKMIHQHLSSR | | |
| | THGSEDS | | |
| IL-21 | QGQDFHMIRMRQLIDIVDQLKNYVNDLVPEFLPAPEDVETNC | 31.9 | 531 |
| v0.40 | EWSAFSCFQKAQLKSANTGNNERIINVSIKKLARKPPSTNAG | | |
| | RRQKHRLTCPSCDSYEKKPPKEFLERFKSLLQKMIHQHLSSR | | |
| | THGSEDS | | |
| IL-21 | QGQDFHMIRMRQLIDIVDQLKNYVNDLVPEFLPAPEDVETNC | 86.9 | 532 |
| v0.41 | EWSAFSCFQKAQLKSANTGNNERIINVSIKKLERKPPSTNAG | | |
| | RRQKHRLTCPSCDSYEKKPPKEFLERFKSLLQKMIHQHLSSR | | |
| | THGSEDS | | |
| IL-21 | QGQDRHMIRMRQLIDIVDQLKNYVNDLVPEFLPAPEDVETNC | >10000 | 533 |
| v0.42 | EWSAFSCFQKAQLKSANTGNNERIINVSIKALKFKPPSTNAG | | |
| | RRQKHRLTCPSCDSYEKKPPKEFLERFKSLLQKMIHQHLSSR | | |
| | THGSEDS | | |
| IL-21 | QGQDRHMIRMRQLIDIVDQLKNYVNDLVPEFLPAPEDVETNC | NB | 534 |
| v0.43 | EWSAFSCFQKAQLKSANTGNNERIINVSIKELKFKPPSTNAG | | |
| | RRQKHRLTCPSCDSYEKKPPKEFLERFKSLLQKMIHQHLSSR | | |
| | THGSEDS | | |
| IL-21 | QGQDRHMIAMRQLIDIVDQLKNYVNDLVPEFLPAPEDVETNC | 4982 | 535 |
| v0.44 | EWSAFSCFQKAQLKSANTGNNERIINVSIKKLKRKPPSTNAG | | |
| | RRQKHRLTCPSCDSYEKKPPKEFLERFKSLLQKMIHQHLSSR | | |
| | THGSEDS | | |
| IL-21 | QGQDRHMIDMRQLIDIVDQLKNYVNDLVPEFLPAPEDVETNC | >10000 | 536 |
| v0.45 | EWSAFSCFQKAQLKSANTGNNERIINVSIKKLKRKPPSTNAG | | |
| | RRQKHRLTCPSCDSYEKKPPKEFLERFKSLLQKMIHQHLSSR | | |
| | THGSEDS | | |
| IL-21 | QGQDRHMIEMRQLIDIVDQLKNYVNDLVPEFLPAPEDVETNC | >10000 | 537 |
| v0.46 | EWSAFSCFQKAQLKSANTGNNERIINVSIKKLKRKPPSTNAG | | |
| | RRQKHRLTCPSCDSYEKKPPKEFLERFKSLLQKMIHQHLSSR | | |
| | THGSEDS | | |
| IL-21 | QGQDRHMIHMRQLIDIVDQLKNYVNDLVPEFLPAPEDVETNC | 11.9 | 538 |
| v0.47 | EWSAFSCFQKAQLKSANTGNNERIINVSIKKLKRKPPSTNAG | | |
| | RRQKHRLTCPSCDSYEKKPPKEFLERFKSLLQKMIHQHLSSR | | |
| | THGSEDS | | |
| IL-21 | QGQDRHMISMRQLIDIVDQLKNYVNDLVPEFLPAPEDVETNC | 3932 | 539 |
| v0.48 | EWSAFSCFQKAQLKSANTGNNERIINVSIKKLKRKPPSTNAG | | |
| | RRQKHRLTCPSCDSYEKKPPKEFLERFKSLLQKMIHQHLSSR | | |
| | THGSEDS | | |
| IL-21 | QGQDRHMITMRQLIDIVDQLKNYVNDLVPEFLPAPEDVETNC | 11664 | 540 |
| v0.49 | EWSAFSCFQKAQLKSANTGNNERIINVSIKKLKRKPPSTNAG | | |
| | RRQKHRLTCPSCDSYEKKPPKEFLERFKSLLQKMIHQHLSSR | | |
| | THGSEDS | | |
| IL-21 | QGQDRHMINMRQLIDIVDQLKNYVNDLVPEFLPAPEDVETNC | 4159 | 541 |
| v0.50 | EWSAFSCFQKAQLKSANTGNNERIINVSIKKLKRKPPSTNAG | | |
| | RRQKHRLTCPSCDSYEKKPPKEFLERFKSLLQKMIHQHLSSR | | |
| | THGSEDS | | |
| IL-21 | QGQDRHMIGMRQLIDIVDQLKNYVNDLVPEFLPAPEDVETNC | 7677 | 542 |
| v0.51 | EWSAFSCFQKAQLKSANTGNNERIINVSIKKLKRKPPSTNAG | | |
| | RRQKHRLTCPSCDSYEKKPPKEFLERFKSLLQKMIHQHLSSR | | |
| | THGSEDS | | |
| IL-21 | QGQDRHMIVMRQLIDIVDQLKNYVNDLVPEFLPAPEDVETNC | 11082 | 543 |
| v0.52 | EWSAFSCFQKAQLKSANTGNNERIINVSIKKLKRKPPSTNAG | | |
| | RRQKHRLTCPSCDSYEKKPPKEFLERFKSLLQKMIHQHLSSR | | |
| | THGSEDS | | |
| IL-21 | QGQDRHMIIMRQLIDIVDQLKNYVNDLVPEFLPAPEDVETNC | >10000 | 544 |
| v0.53 | EWSAFSCFQKAQLKSANTGNNERIINVSIKKLKRKPPSTNAG | | |
| | RRQKHRLTCPSCDSYEKKPPKEFLERFKSLLQKMIHQHLSSR | | |
| | THGSEDS | | |
| IL-21 | QGQDRHMILMRQLIDIVDQLKNYVNDLVPEFLPAPEDVETNC | 7168 | 545 |
| v0.54 | EWSAFSCFQKAQLKSANTGNNERIINVSIKKLKRKPPSTNAG | | |
| | RRQKHRLTCPSCDSYEKKPPKEFLERFKSLLQKMIHQHLSSR | | |
| | THGSEDS | | |
| IL-21 | QGQDRHMIYMRQLIDIVDQLKNYVNDLVPEFLPAPEDVETNC | 675.7 | 546 |
| v0.55 | EWSAFSCFQKAQLKSANTGNNERIINVSIKKLKRKPPSTNAG | | |
| | RRQKHRLTCPSCDSYEKKPPKEFLERFKSLLQKMIHQHLSSR | | |
| | THGSEDS | | |
| IL-21 | QGQDRHMIRMRQLIDIVDQLKNYVNDLVPEFLPAPEDVETNC | 3431 | 547 |
| v0.56 | EWSAFSCFQKAQLKSANTGNNERIINVSIAKLKFKPPSTNAG | | |
| | RRQKHRLTCPSCDSYEKKPPKEFLERFKSLLQKMIHQHLSSR | | |
| | THGSEDS | | |
| IL-21 | QGQDRHMIRMRQLIDIVDQLKNYVNDLVPEFLPAPEDVETNC | 5041 | 548 |
| v0.57 | EWSAFSCFQKAQLKSANTGNNERIINVSIKKLAFKPPSTNAG | | |
| | RRQKHRLTCPSCDSYEKKPPKEFLERFKSLLQKMIHQHLSSR | | |
| | THGSEDS | | |
| IL-21 | QGQDRHMIRMRQLIDIVDQLKNYVNDLVPEFLPAPEDVETNC | 4760 | 549 |
| v0.58 | EWSAFSCFQKAQLKSANTGNNERIINVSIKKLKFAPPSTNAG | | |
| | RRQKHRLTCPSCDSYEKKPPKEFLERFKSLLQKMIHQHLSSR | | |
| | THGSEDS | | |
| IL-21 | QGQDRHMIRMRQLIDIVDQLKNYVNDLVPEFLPAPEDVETNC | NB | 550 |
| v0.59 | EWSAFSCFQKAQLKSANTGNNERIINVSIKKLEFKPPSTNAG | | |
| | RRQKHRLTCPSCDSYEKKPPKEFLERFKSLLQKMIHQHLSSR | | |
| | THGSEDS | | |

NB = no binding at the highest concentration tested

### Combination of Mutations

In some embodiments, an IL-21 polypeptide as described herein comprises a combination of (a) mutations that alter its isoelectric point relative to a human IL-21 (SEQ ID NO: 390) and (b) mutations that attenuate its binding to IL-21R, relative to the human IL-21.

In some embodiments, a mutation in group (a) is at a position selected from positions: K56, S80, T81, N82, A83, G84, R85, R86, Q87, K88, H89, R90, L91, and T92; and a mutation in group (b) is at a position selected from the group consisting of: R5, I8, R9, R11, Q12, 114, D15, D18, Q19, Y23, R65, S70, K72, K73, K75, R76, K77, Q116, and K117, wherein the position numbering is according to the amino acid sequence of SEQ ID NO: 390. In some embodiments, a mutation in group (a) is at a position selected from positions: K56, S80, T81, N82, A83, G84, R85, R86, Q87, K88, H89, R90, L91, and T92; and a mutation in group (b) is at a position selected from the group consisting of: R5, I8, R9, R11, L13, I14, I16, V17, D18, K72, K73, L74, K75, R76, K77, and K117, wherein the position numbering is according to the amino acid sequence of SEQ ID NO: 390.

In some embodiments, a mutation in group (a) is at a position selected from positions: S80, T81, N82, A83, G84, R85, R86, Q87, K88, H89, R90, L91, and T92; and a mutation in group (b) is at a position selected from the group consisting of: R5, I8, R9, R11, L13, I14, I16, V17, D18, K72, K73, L74, K75, R76, K77, and K117, wherein the position numbering is according to the amino acid sequence of SEQ ID NO: 390. In some embodiments, a mutation in group (a) is at a position selected from positions: S80, T81, N82, A83, R85, R86, Q87, K88, H89, R90, L91, and T92; and a mutation in group (b) is at a position selected from the group consisting of: R5, I8, R9, R11, L13, I14, I16, V17, D18, K72, K73, L74, K75, R76, K77, and K117, wherein the position numbering is according to the amino acid sequence of SEQ ID NO: 390. In some embodiments, a mutation in group (a) is at a position selected from positions: S80, T81, N82, A83, G84, R85, R86, Q87, K88, H89, R90, L91, and T92; and a mutation in group (b) is at position R76, wherein the position numbering is according to the amino acid sequence of SEQ ID NO: 390.

In some embodiments, an IL-21 polypeptide comprises a consensus sequence as follows: QGQDX₁HMX₂X₃MX₄QX₅X₆DX₇X₈X₉QLKNYVNDLVPEFLPAPEDVETNCEWSAFSCF QKAQLX₁₀SANTGNNERIINVSIX₁₁X₁₂X₁₃X₁₄X₁₅X₁₆PPX₁₇X₁₈X₁₉X₂₀X₂₁X₂₂X₂₃X₂₄X₂₅X₂₆X₂₇X₂₈X₂₉CPSCDSYEKKPPKEFLERFKSLLQ X₃₀MIHQHLSSRTHGSEDS (SEQ ID NO: 556), wherein X₁=F, A, E, S, T, N, Q, V, I, L, Y, R; X₂=E, I; X₃=A, D, E, H, S, T, N, G, V, I, L, Y, R; X₄=D, E, R; X₅=F, R, L; X₆=D, I; X₇=A, S, R, I; X₈=I, A, V; X₉=A, D; X₁₀=A, E, K; X₁₁=A, E, K; X₁₂=A, E, K; X₁₃=I, F, M, L; X₁₄=A, K, E; X₁₅=E, F, A, N, D, S, T, Q, V, I, L, Y, M, R; X₁₆=A, E, K; X₁₇=G, S; X₁₈=G, T; X₁₉=G, E, N; X₂₀=G, S, E, A; X₂₁=E, G; X₂₂=G, E, S, R; X₂₃=G, E, R; X₂₄=S, G, E, Q; X₂₅=G, K; X₂₆=G, S, H; X₂₇=A, E, S, G, R; X₂₈=S, G, L; X₂₉=G, S, T; X₃₀=A, K, provided at least one of X₁-X₉X₁₁-X₁₆ and X₃₀ is not the amino acid residue at the identical position set forth in SEQ ID NO: 390 and reduces binding to IL-21R, and provided at least one of X₁₀ and X₁₇-X₂₉ is not the amino acid residue at the identical position set forth in SEQ ID NO: 390 and reduces the isoelectric point relative to human IL-21 (SEQ ID NO: 390).

In some embodiments, an IL-21 polypeptide comprising a combination of mutations comprises at least one mutation in a position of SEQ ID NO: 391. In some embodiments, an IL-21 polypeptide comprising a combination of mutations comprises at least one mutation in a position selected from the group consisting of positions: R5, I8, R9, R1, Q12, 114, D15, D18, Q19, Y23, R65, S70, K72, K73, K75, R76, K77, S80, Q116, and K117, wherein the position numbering is according to SEQ ID NO: 391. In some embodiments, an IL-21 polypeptide comprising a combination of mutations comprises at least one mutation in a position selected from the group consisting of positions: R5, I8, R9, R11, L13, I14, I16, V17, D18, K72, K73, L74, K75, R76, K77, and K117, wherein the position numbering is according to SEQ ID NO: 391.

In some embodiments, an IL-21 polypeptide comprising a combination of mutations comprises at least one mutation in a position of SEQ ID NO: 421. In some embodiments, an IL-21 polypeptide comprising a combination of mutations comprises at least one mutation in a position selected from the group consisting of positions: R5, I8, R9, R1, Q12, 114, D15, D18, Q19, Y23, R65, S70, K72, K73, K75, R76, K77, S80, Q116, and K117, wherein the position numbering is according to SEQ ID NO: 421. In some embodiments, an IL-21 polypeptide comprising a combination of mutations comprises at least one mutation in a position selected from the group consisting of positions: R5, I8, R9, R11, L13, I14, I16, V17, D18, K72, K73, L74, K75, R76, K77, and K117, wherein the position numbering is according to SEQ ID NO: 421.

In some embodiments, an IL-21 polypeptide comprises (i) four or more amino acid substitutions providing a reduced isoelectric point relative to a human IL-21 polypeptide comprising SEQ ID NO: 390, wherein the four or more amino acid substitutions are within S80 to T92 of SEQ ID NO: 390, wherein at least four amino acid substitutions are at residues R85, R86, K88, H89, R90, or a combination thereof, and (ii) at least one amino acid substitution selected from (a) R11D; (b) R11E; (c) I14D, D18A and K117A; (d) R76E; (e) R5F; (f) R76F; (g) 18E; (h) R5A; (i) R5E; (j) R5S; (k) R5T; (1) R5N; (m) R5Q; (n) R5V; (o) R51; (p) R5L; (q) R5Y; (r) R76A; (s) R76N; (t) R76D; (u) R76S; (v) R76T; (w) R76Q; (x) R76V; (y) R761; (z) R76L; (aa) R76Y; (bb) K77A; (cc) K77E; (dd) K72A; (ee) K72E; (ff) K75A; (gg) K75E; (hh) K73A; (ii) K73E; (jj) R5F and K77A; (kk) R5F and K77E; (11) R5F and K72A; (mm) R5F and K72E; (nn) R5F and K76A; (oo) R5F and K76E; (pp) K73A and K76F; (qq) K73E and K76F; (rr) R9A; (ss) R9D; (tt) R9E; (uu) R9H; (vv) R9S; (ww) R9T; (xx) R9N; (zz) R9G; (aaa) R9V; (bbb) R91; (ccc) R9L; (ddd) R9Y; (eee) K72A and R76F; (fff) K75A and R76F; (ggg) R76F and K77A; (hhh) K75E and R76F; (iii) V171 and L741; (jjj) 116A and L74F; (kkk) 116S, V171, and L74V; (111) 116R, V171, and L74I; (mmm) L13F, 116A, V17A and L74M; and (nnn) L13R, 116A, V171 and L74I. In some embodiments, the at least one amino acid substitution is RIID. In some embodiments, the at least one amino acid substitution is R11E. In some embodiments, the at least one amino acid substitution is I14D, D18A and K117A. In some embodiments, the at least one amino acid substitution is R76E. In some embodiments, the at least one amino acid substitution is R5F. In some embodiments, the at least one amino acid substitution is R76F. In some embodiments, the at least one amino acid substitution is 18E. In some embodiments, the at least one amino acid substitution is R5A. In some embodiments, the at least one amino acid substitution is R5E. In some embodiments, the at least one amino acid substitution is R5S. In some embodiments, the at least one amino acid substitution is R5T. In some embodiments, the at least one amino acid substitution is R5N. In some embodiments, the at least one amino acid substitution is R5Q. In some embodiments, the at least one amino acid substitution is R5V. In some embodiments, the at least one amino acid substitution is R5I. In some embodiments, the at least one amino acid substitution is R5L. In some embodiments, the at least one amino acid substitution is R5Y. In some embodiments, the at least one amino acid substitution is R76A. In some embodiments, the at least one amino acid substitution is R76N. In some embodiments, the at least one amino acid substitution is R76D. In some embodiments, the at least one amino acid substitution is R76S. In some embodiments, the at least one amino acid substitution is R76T. In some embodiments, the at least one amino acid substitution is R76Q. In some embodiments, the at least one amino acid substitution is R76V. In some embodiments, the at least one amino acid substitution is R76I. In some embodiments, the at least one amino acid substitution is R76L. In some embodiments, the at least one amino acid substitution is R76Y. In some embodiments, the at least one amino acid substitution is K77A. In some embodiments, the at least one amino acid substitution is K77E. In some embodiments, the at least one amino acid substitution is K72A. In some embodiments, the at least one amino acid substitution is K72E. In some embodiments, the at least one amino acid substitution is K75A. In some embodiments, the at least one amino acid substitution is K75E. In some embodiments, the at least one amino acid substitution is K73A. In some embodiments, the at least one amino acid substitution is K73E. In some embodiments, the at least one amino acid substitution is R5F and K77A. In some embodiments, the at least one amino acid substitution is R5F and K77E. In some embodiments, the at least one amino acid substitution is R5F and K72A. In some embodiments, the at least one amino acid substitution is R5F and K72E. In some embodiments, the at least one amino acid substitution is R5F and K76A. In some embodiments, the at least one amino acid substitution is R5F and K76E. In some embodiments, the at least one amino acid substitution is K73A and K76F. In some embodiments, the at least one amino acid substitution is K73E and K76F. In some embodiments, the at least one amino acid substitution is R9A. In some embodiments, the at least one amino acid substitution is R9D. In some embodiments, the at least one amino acid substitution is R9E. In some embodiments, the at least one amino acid substitution is R9H. In some embodiments, the at least one amino acid substitution is R9S. In some embodiments, the at least one amino acid substitution is R9T. In some embodiments, the at least one amino acid substitution is R9N. In some embodiments, the at least one amino acid substitution is R9G. In some embodiments, the at least one amino acid substitution is R9V. In some embodiments, the at least one amino acid substitution is R9I. In some embodiments, the at least one amino acid substitution is R9L. In some embodiments, the at least one amino acid substitution is R9Y. In some embodiments, the at least one amino acid substitution is K72A and R76F. In some embodiments, the at least one amino acid substitution is K75A and R76F. In some embodiments, the at least one amino acid substitution is R76F and K77A. In some embodiments, the at least one amino acid substitution is K75E and R76F. In some embodiments, the at least one amino acid substitution is V171 and L74I. In some embodiments, the at least one amino acid substitution is I16A and L74F. In some embodiments, the at least one amino acid substitution is I16S, V171, and L74V. In some embodiments, the at least one amino acid substitution is I16R, V171, and L741. In some embodiments, the at least one amino acid substitution is L13F, 116A, V17A and L74M. In some embodiments, the at least one amino acid substitution is L13R, 116A, V171 and L741.

In some embodiments, an IL-21 polypeptide comprises (i) an amino acid sequence selected from SEQ ID Nos: 391-421, and (ii) at least one amino acid substitution selected from (a) RI1D; (b) RI1E; (c) I14D, D18A and K117A; (d) R76E; (e) R5F; (f) R76F; (g) 18E; (h) R5A; (i) R5E; (j) R5S; (k) R5T; (1) R5N; (m) R5Q; (n) R5V; (o) R5I; (p) R5L; (q) R5Y; (r) R76A; (s) R76N; (t) R76D; (u) R76S; (v) R76T; (w) R76Q; (x) R76V; (y) R76I; (z) R76L; (aa) R76Y; (bb) K77A; (cc) K77E; (dd) K72A; (ee) K72E; (ff) K75A; (gg) K75E; (hh) K73A; (ii) K73E; (jj) R5F and K77A; (kk) R5F and K77E; (11) R5F and K72A; (mm) R5F and K72E; (nn) R5F and K76A; (oo) R5F and K76E; (pp) K73A and K76F; (qq) K73E and K76F; (rr) R9A; (ss) R9D; (tt) R9E; (uu) R9H; (vv) R9S; (ww) R9T; (xx) R9N; (zz) R9G; (aaa) R9V; (bbb) R9I; (ccc) R9L; (ddd) R9Y; (eee) K72A and R76F; (fff) K75A and R76F; (ggg) R76F and K77A; (hhh) K75E and R76F; (iii) V171 and L741; (jjj) 116A and L74F; (kkk) I16S, V17I, and L74V; (111) I16R, V17I, and L74I; (mmm) L13F, I16A, V17A and L74M; and (nnn) L13R, 116A, V171 and L741.

Exemplary IL-21 polypeptides comprising at least one amino acid mutation that reduces the isoelectric point and at least one amino acid mutation that reduces affinity to a human IL-21 receptor are provided in Table 9.

In some embodiments, an IL-21 polypeptide comprises an amino acid sequence selected from SEQ ID NOs: 422-491. In some embodiments, an IL-21 polypeptide comprises an amino acid sequence selected from SEQ ID NOs: 422-486. In some embodiments, an IL-21 polypeptide comprises an amino acid sequence selected from SEQ ID NOs: 487-491.

In some embodiments, an IL-21 polypeptide comprises an amino acid sequence at least 75%, 80%, 85%, 90%, 91%, 92%, 93%, 94%, 95%, 96%, 97%, 98% or 99% identical to an amino acid sequence selected from SEQ ID NOs: 422-491. In some embodiments, an IL-21 polypeptide comprises an amino acid sequence at least 75%, 80%, 85%, 90%, 91%, 92%, 93%, 94%, 95%, 96%, 97%, 98% or 99% identical to an amino acid sequence selected from SEQ ID NOs: 422-486. In some embodiments, an IL-21 polypeptide comprises an amino acid sequence at least 75%, 80%, 85%, 90%, 91%, 92%, 93%, 94%, 95%, 96%, 97%, 98% or 99% identical to an amino acid sequence selected from SEQ ID NOs: 487-491.

In some embodiments, an IL-21 polypeptide comprises the amino acid sequence of SEQ ID NO: 422. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 422.

In some embodiments, an IL-21 polypeptide comprises the amino acid sequence of SEQ ID NO: 423. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 423.

In some embodiments, an IL-21 polypeptide comprises the amino acid sequence of SEQ ID NO: 424. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 424.

In some embodiments, an IL-21 polypeptide comprises the amino acid sequence of SEQ ID NO: 425. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 425.

In some embodiments, an IL-21 polypeptide comprises the amino acid sequence of SEQ ID NO: 426. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 426.

In some embodiments, an IL-21 polypeptide comprises the amino acid sequence of SEQ ID NO: 427. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 427.

In some embodiments, an IL-21 polypeptide comprises the amino acid sequence of SEQ ID NO: 428. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 428.

In some embodiments, an IL-21 polypeptide comprises the amino acid sequence of SEQ ID NO: 429. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 429.

In some embodiments, an IL-21 polypeptide comprises the amino acid sequence of SEQ ID NO: 430. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 430.

In some embodiments, an IL-21 polypeptide comprises the amino acid sequence of SEQ ID NO: 431. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 431.

In some embodiments, an IL-21 polypeptide comprises the amino acid sequence of SEQ ID NO: 432. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 432.

In some embodiments, an IL-21 polypeptide comprises the amino acid sequence of SEQ ID NO: 433. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 433.

In some embodiments, an IL-21 polypeptide comprises the amino acid sequence of SEQ ID NO: 434. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 434.

In some embodiments, an IL-21 polypeptide comprises the amino acid sequence of SEQ ID NO: 435. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 435.

In some embodiments, an IL-21 polypeptide comprises the amino acid sequence of SEQ ID NO: 436. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 436.

In some embodiments, an IL-21 polypeptide comprises the amino acid sequence of SEQ ID NO: 437. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 437.

In some embodiments, an IL-21 polypeptide comprises the amino acid sequence of SEQ ID NO: 438. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 438.

In some embodiments, an IL-21 polypeptide comprises the amino acid sequence of SEQ ID NO: 439. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 439.

In some embodiments, an IL-21 polypeptide comprises the amino acid sequence of SEQ ID NO: 440. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 440.

In some embodiments, an IL-21 polypeptide comprises the amino acid sequence of SEQ ID NO: 441. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 441.

In some embodiments, an IL-21 polypeptide comprises the amino acid sequence of SEQ ID NO: 442. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 442.

In some embodiments, an IL-21 polypeptide comprises the amino acid sequence of SEQ ID NO: 443. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 443.

In some embodiments, an IL-21 polypeptide comprises the amino acid sequence of SEQ ID NO: 444. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 444.

In some embodiments, an IL-21 polypeptide comprises the amino acid sequence of SEQ ID NO: 445. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 445.

In some embodiments, an IL-21 polypeptide comprises the amino acid sequence of SEQ ID NO: 446. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 446.

In some embodiments, an IL-21 polypeptide comprises the amino acid sequence of SEQ ID NO: 447. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 447.

In some embodiments, an IL-21 polypeptide comprises the amino acid sequence of SEQ ID NO: 448. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 448.

In some embodiments, an IL-21 polypeptide comprises the amino acid sequence of SEQ ID NO: 449. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 449.

In some embodiments, an IL-21 polypeptide comprises the amino acid sequence of SEQ ID NO: 450. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 450.

In some embodiments, an IL-21 polypeptide comprises the amino acid sequence of SEQ ID NO: 451. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 451.

In some embodiments, an IL-21 polypeptide comprises the amino acid sequence of SEQ ID NO: 452. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 452.

In some embodiments, an IL-21 polypeptide comprises the amino acid sequence of SEQ ID NO: 453. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 453.

In some embodiments, an IL-21 polypeptide comprises the amino acid sequence of SEQ ID NO: 454. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 454.

In some embodiments, an IL-21 polypeptide comprises the amino acid sequence of SEQ ID NO: 455. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 455.

In some embodiments, an IL-21 polypeptide comprises the amino acid sequence of SEQ ID NO: 456. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 456.

In some embodiments, an IL-21 polypeptide comprises the amino acid sequence of SEQ ID NO: 457. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 457.

In some embodiments, an IL-21 polypeptide comprises the amino acid sequence of SEQ ID NO: 458. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 458.

In some embodiments, an IL-21 polypeptide comprises the amino acid sequence of SEQ ID NO: 459. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 459.

In some embodiments, an IL-21 polypeptide comprises the amino acid sequence of SEQ ID NO: 460. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 460.

In some embodiments, an IL-21 polypeptide comprises the amino acid sequence of SEQ ID NO: 61. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 461.

In some embodiments, an IL-21 polypeptide comprises the amino acid sequence of SEQ ID NO: 462. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 462.

In some embodiments, an IL-21 polypeptide comprises the amino acid sequence of SEQ ID NO: 463. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 463.

In some embodiments, an IL-21 polypeptide comprises the amino acid sequence of SEQ ID NO: 464. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 464.

In some embodiments, an IL-21 polypeptide comprises the amino acid sequence of SEQ ID NO: 465. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 465.

In some embodiments, an IL-21 polypeptide comprises the amino acid sequence of SEQ ID NO: 466. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 466.

In some embodiments, an IL-21 polypeptide comprises the amino acid sequence of SEQ ID NO: 467. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 467.

In some embodiments, an IL-21 polypeptide comprises the amino acid sequence of SEQ ID NO: 468. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 468.

In some embodiments, an IL-21 polypeptide comprises the amino acid sequence of SEQ ID NO: 469. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 469.

In some embodiments, an IL-21 polypeptide comprises the amino acid sequence of SEQ ID NO: 470. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 470.

In some embodiments, an IL-21 polypeptide comprises the amino acid sequence of SEQ ID NO: 471. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 471.

In some embodiments, an IL-21 polypeptide comprises the amino acid sequence of SEQ ID NO: 472. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 472.

In some embodiments, an IL-21 polypeptide comprises the amino acid sequence of SEQ ID NO: 473. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 473.

In some embodiments, an IL-21 polypeptide comprises the amino acid sequence of SEQ ID NO: 474. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 474.

In some embodiments, an IL-21 polypeptide comprises the amino acid sequence of SEQ ID NO: 475. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 475.

In some embodiments, an IL-21 polypeptide comprises the amino acid sequence of SEQ ID NO: 476. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 476.

In some embodiments, an IL-21 polypeptide comprises the amino acid sequence of SEQ ID NO: 477. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 477.

In some embodiments, an IL-21 polypeptide comprises the amino acid sequence of SEQ ID NO: 478. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 478.

In some embodiments, an IL-21 polypeptide comprises the amino acid sequence of SEQ ID NO: 479. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 479.

In some embodiments, an IL-21 polypeptide comprises the amino acid sequence of SEQ ID NO: 480. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 480.

In some embodiments, an IL-21 polypeptide comprises the amino acid sequence of SEQ ID NO: 481. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 481.

In some embodiments, an IL-21 polypeptide comprises the amino acid sequence of SEQ ID NO: 482. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 482.

In some embodiments, an IL-21 polypeptide comprises the amino acid sequence of SEQ ID NO: 483. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 483.

In some embodiments, an IL-21 polypeptide comprises the amino acid sequence of SEQ ID NO: 484. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 484.

In some embodiments, an IL-21 polypeptide comprises the amino acid sequence of SEQ ID NO: 485. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 485.

In some embodiments, an IL-21 polypeptide comprises the amino acid sequence of SEQ ID NO: 486. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 486.

In some embodiments, an IL-21 polypeptide comprises the amino acid sequence of SEQ ID NO: 487. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 487.

In some embodiments, an IL-21 polypeptide comprises the amino acid sequence of SEQ ID NO: 488. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 488.

In some embodiments, an IL-21 polypeptide comprises the amino acid sequence of SEQ ID NO: 489. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 489.

In some embodiments, an IL-21 polypeptide comprises the amino acid sequence of SEQ ID NO: 490. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 490.

In some embodiments, an IL-21 polypeptide comprises the amino acid sequence of SEQ ID NO: 491. In some embodiments, an IL-21 polypeptide comprises an amino acid sequence at least about 75%, 80%, 85%, 90%, 91%, 92%, 93%, 94%, 95%, 96%, 97%, 98% or 99% identical to SEQ ID NO: 491.

TABLE 9
IL-21 Polypeptides with Reduced Isoelectric Point and Reduced Affinity

| | | K_{D} at | |
|---|---|---|---|
| IL-21 | | 37° C. | SEQ |
| Mutein | Amino Acid Sequence | (nM) | ID NO |
| IL-21 | QGQDRHMIRMDQLIDIVDQLKNYVNDLVPEFLPAPEDVETNCEW | ND | 422 |
| v2.1 | SAFSCFQKAQLKSANTGNNERIINVSIKKLKRKPPSTNAGGGQG | | |
| | HELTCPSCDSYEKKPPKEFLERFKSLLQKMIHQHLSSRTHGSED | | |
| | S | | |
| IL-21 | QGQDRHMIRMEQLIDIVDQLKNYVNDLVPEFLPAPEDVET | ND | 423 |
| v2.2 | NCEWSAFSCFQKAQLKSANTGNNERIINVSIKKLKRKPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLDDIVAQLKNYVNDLVPEFLPAPEDVET | ND | 424 |
| v2.3 | NCEWSAFSCFQKAQLKSANTGNNERIINVSIKKLKRKPPS | | |
| | TNAGGGQGHELTCPSCDSYEKKPPKEFLERFKSLLQAMIH | | |
| | QHLSSRTHGSEDS | | |
| IL-21 | QGQDRHMIRMRQLIDIVDQLKNYVNDLVPEFLPAPEDVET | >5000 | 425 |
| v2.4 | NCEWSAFSCFQKAQLKSANTGNNERIINVSIKKLKEKPPS | | |
| | TNAGGGQGHELTCPSCDSYEKKPPKEFLERFKSLLQKMIH | | |
| | QHLSSRTHGSEDS | | |
| IL-21 | QGQDFHMIRMRQLIDIVDQLKNYVNDLVPEFLPAPEDVET | ND | 426 |
| v2.5 | NCEWSAFSCFQKAQLKSANTGNNERIINVSIKKLKRKPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLIDIVDQLKNYVNDLVPEFLPAPEDVET | 1700 | 427 |
| v2.6 | NCEWSAFSCFQKAQLKSANTGNNERIINVSIKKLKFKPPS | | |
| | TNAGGGQGHELTCPSCDSYEKKPPKEFLERFKSLLQKMIH | | |
| | QHLSSRTHGSEDS | | |
| IL-21 | QGQDRHMERMRQLIDIVDQLKNYVNDLVPEFLPAPEDVET | 156 | 428 |
| v2.7 | NCEWSAFSCFQKAQLKSANTGNNERIINVSIKKLKRKPPS | | |
| | TNAGGGQGHELTCPSCDSYEKKPPKEFLERFKSLLQKMIH | | |
| | QHLSSRTHGSEDS | | |
| IL-21 | QGQDAHMIRMRQLIDIVDQLKNYVNDLVPEFLPAPEDVET | 18.9 | 429 |
| v2.8 | NCEWSAFSCFQKAQLKSANTGNNERIINVSIKKLKRKPPS | | |
| | TNAGGGQGHELTCPSCDSYEKKPPKEFLERFKSLLQKMIH | | |
| | QHLSSRTHGSEDS | | |
| IL-21 | QGQDEHMIRMRQLIDIVDQLKNYVNDLVPEFLPAPEDVET | 420 | 430 |
| v2.9 | NCEWSAFSCFQKAQLKSANTGNNERIINVSIKKLKRKPPS | | |
| | TNAGGGQGHELTCPSCDSYEKKPPKEFLERFKSLLQKMIH | | |
| | QHLSSRTHGSEDS | | |
| IL-21 | QGQDSHMIRMRQLIDIVDQLKNYVNDLVPEFLPAPEDVET | 24.4 | 431 |
| v2.10 | NCEWSAFSCFQKAQLKSANTGNNERIINVSIKKLKRKPPS | | |
| | TNAGGGQGHELTCPSCDSYEKKPPKEFLERFKSLLQKMIH | | |
| | QHLSSRTHGSEDS | | |
| IL-21 | QGQDTHMIRMRQLIDIVDQLKNYVNDLVPEFLPAPEDVET | 9.4 | 432 |
| v2.11 | NCEWSAFSCFQKAQLKSANTGNNERIINVSIKKLKRKPPS | | |
| | TNAGGGQGHELTCPSCDSYEKKPPKEFLERFKSLLQKMIH | | |
| | QHLSSRTHGSEDS | | |
| IL-21 | QGQDNHMIRMRQLIDIVDQLKNYVNDLVPEFLPAPEDVET | 54.6 | 433 |
| v2.12 | NCEWSAFSCFQKAQLKSANTGNNERIINVSIKKLKRKPPS | | |
| | TNAGGGQGHELTCPSCDSYEKKPPKEFLERFKSLLQKMIH | | |
| | QHLSSRTHGSEDS | | |
| IL-21 | QGQDQHMIRMRQLIDIVDQLKNYVNDLVPEFLPAPEDVET | 15.2 | 434 |
| v2.13 | NCEWSAFSCFQKAQLKSANTGNNERIINVSIKKLKRKPPS | | |
| | TNAGGGQGHELTCPSCDSYEKKPPKEFLERFKSLLQKMIH | | |
| | QHLSSRTHGSEDS | | |
| IL-21 | QGQDVHMIRMRQLIDIVDQLKNYVNDLVPEFLPAPEDVET | 6.5 | 435 |
| v2.14 | NCEWSAFSCFQKAQLKSANTGNNERIINVSIKKLKRKPPS | | |
| | TNAGGGQGHELTCPSCDSYEKKPPKEFLERFKSLLQKMIH | | |
| | QHLSSRTHGSEDS | | |
| IL-21 | QGQDIHMIRMRQLIDIVDQLKNYVNDLVPEFLPAPEDVET | 6.2 | 436 |
| v2.15 | NCEWSAFSCFQKAQLKSANTGNNERIINVSIKKLKRKPPS | | |
| | TNAGGGQGHELTCPSCDSYEKKPPKEFLERFKSLLQKMIH | | |
| | QHLSSRTHGSEDS | | |
| IL-21 | QGQDLHMIRMRQLIDIVDQLKNYVNDLVPEFLPAPEDVET | 2.1 | 437 |
| v2.16 | NCEWSAFSCFQKAQLKSANTGNNERIINVSIKKLKRKPPS | | |
| | TNAGGGQGHELTCPSCDSYEKKPPKEFLERFKSLLQKMIH | | |
| | QHLSSRTHGSEDS | | |
| IL-21 | QGQDYHMIRMRQLIDIVDQLKNYVNDLVPEFLPAPEDVET | 9.8 | 438 |
| v2.17 | NCEWSAFSCFQKAQLKSANTGNNERIINVSIKKLKRKPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLIDIVDQLKNYVNDLVPEFLPAPEDVET | 3180 | 439 |
| v2.18 | NCEWSAFSCFQKAQLKSANTGNNERIINVSIKKLKAKPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLIDIVDQLKNYVNDLVPEFLPAPEDVET | 2310 | 440 |
| v2.19 | NCEWSAFSCFQKAQLKSANTGNNERIINVSIKKLKNKPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLIDIVDQLKNYVNDLVPEFLPAPEDVET | NB | 441 |
| v2.20 | NCEWSAFSCFQKAQLKSANTGNNERIINVSIKKLKDKPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLIDIVDQLKNYVNDLVPEFLPAPEDVET | 3800 | 442 |
| v2.21 | NCEWSAFSCFQKAQLKSANTGNNERIINVSIKKLKSKPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLIDIVDQLKNYVNDLVPEFLPAPEDVET | 3030 | 443 |
| v2.22 | NCEWSAFSCFQKAQLKSANTGNNERIINVSIKKLKTKPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLIDIVDQLKNYVNDLVPEFLPAPEDVET | 2410 | 444 |
| v2.23 | NCEWSAFSCFQKAQLKSANTGNNERIINVSIKKLKQKPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLIDIVDQLKNYVNDLVPEFLPAPEDVET | 5180 | 445 |
| v2.24 | NCEWSAFSCFQKAQLKSANTGNNERIINVSIKKLKVKPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLIDIVDQLKNYVNDLVPEFLPAPEDVET | 2990 | 446 |
| v2.25 | NCEWSAFSCFQKAQLKSANTGNNERIINVSIKKLKIKPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLIDIVDQLKNYVNDLVPEFLPAPEDVET | 651 | 447 |
| v2.26 | NCEWSAFSCFQKAQLKSANTGNNERIINVSIKKLKLKPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLIDIVDQLKNYVNDLVPEFLPAPEDVET | 2150 | 448 |
| v2.27 | NCEWSAFSCFQKAQLKSANTGNNERIINVSIKKLKYKPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLIDIVDQLKNYVNDLVPEFLPAPEDVET | 0.693 | 449 |
| v2.28 | NCEWSAFSCFQKAQLKSANTGNNERIINVSIKKLKRAPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLIDIVDQLKNYVNDLVPEFLPAPEDVET | 2.8 | 450 |
| v2.29 | NCEWSAFSCFQKAQLKSANTGNNERIINVSIKKLKREPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLIDIVDQLKNYVNDLVPEFLPAPEDVET | 0.419 | 451 |
| v2.30 | NCEWSAFSCFQKAQLKSANTGNNERIINVSIAKLKRKPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLIDIVDQLKNYVNDLVPEFLPAPEDVET | 3.4 | 452 |
| v2.31 | NCEWSAFSCFQKAQLKSANTGNNERIINVSIEKLKRKPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLIDIVDQLKNYVNDLVPEFLPAPEDVET | 0.236 | 453 |
| v2.32 | NCEWSAFSCFQKAQLKSANTGNNERIINVSIKKLARKPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLIDIVDQLKNYVNDLVPEFLPAPEDVET | 1.3 | 454 |
| v2.33 | NCEWSAFSCFQKAQLKSANTGNNERIINVSIKKLERKPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLIDIVDQLKNYVNDLVPEFLPAPEDVET | 2.0 | 455 |
| v2.34 | NCEWSAFSCFQKAQLKSANTGNNERIINVSIKALKRKPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLIDIVDQLKNYVNDLVPEFLPAPEDVET | 449.0 | 456 |
| v2.35 | NCEWSAFSCFQKAQLKSANTGNNERIINVSIKELKRKPPS | | |
| | TNAGGGQGHELTCPSCDSYEKKPPKEFLERFKSLLQKMIH | | |
| | QHLSSRTHGSEDS | | |
| IL-21 | QGQDFHMIRMRQLIDIVDQLKNYVNDLVPEFLPAPEDVET | 125.0 | 457 |
| v2.36 | NCEWSAFSCFQKAQLKSANTGNNERIINVSIKKLKRAPPS | | |
| | TNAGGGQGHELTCPSCDSYEKKPPKEFLERFKSLLQKMIH | | |
| | QHLSSRTHGSEDS | | |
| IL-21 | QGQDFHMIRMRQLIDIVDQLKNYVNDLVPEFLPAPEDVET | 601.0 | 458 |
| v2.37 | NCEWSAFSCFQKAQLKSANTGNNERIINVSIKKLKREPPS | | |
| | TNAGGGQGHELTCPSCDSYEKKPPKEFLERFKSLLQKMIH | | |
| | QHLSSRTHGSEDS | | |
| IL-21 | QGQDFHMIRMRQLIDIVDQLKNYVNDLVPEFLPAPEDVET | 66.9 | 459 |
| v2.38 | NCEWSAFSCFQKAQLKSANTGNNERIINVSIAKLKRKPPS | | |
| | TNAGGGQGHELTCPSCDSYEKKPPKEFLERFKSLLQKMIH | | |
| | QHLSSRTHGSEDS | | |
| IL-21 | QGQDFHMIRMRQLIDIVDQLKNYVNDLVPEFLPAPEDVET | 614.0 | 460 |
| v2.39 | NCEWSAFSCFQKAQLKSANTGNNERIINVSIEKLKRKPPS | | |
| | TNAGGGQGHELTCPSCDSYEKKPPKEFLERFKSLLQKMIH | | |
| | QHLSSRTHGSEDS | | |
| IL-21 | QGQDFHMIRMRQLIDIVDQLKNYVNDLVPEFLPAPEDVET | 54.7 | 461 |
| v2.40 | NCEWSAFSCFQKAQLKSANTGNNERIINVSIKKLARKPPS | | |
| | TNAGGGQGHELTCPSCDSYEKKPPKEFLERFKSLLQKMIH | | |
| | QHLSSRTHGSEDS | | |
| IL-21 | QGQDFHMIRMRQLIDIVDQLKNYVNDLVPEFLPAPEDVET | 104.0 | 462 |
| v2.41 | NCEWSAFSCFQKAQLKSANTGNNERIINVSIKKLERKPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLIDIVDQLKNYVNDLVPEFLPAPEDVET | NB | 463 |
| v2.42 | NCEWSAFSCFQKAQLKSANTGNNERIINVSIKALKFKPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLIDIVDQLKNYVNDLVPEFLPAPEDVET | NB | 464 |
| v2.43 | NCEWSAFSCFQKAQLKSANTGNNERIINVSIKELKFKPPS | | |
| | TNAGGGQGHELTCPSCDSYEKKPPKEFLERFKSLLQKMIH | | |
| | QHLSSRTHGSEDS | | |
| IL-21 | QGQDRHMIAMRQLIDIVDQLKNYVNDLVPEFLPAPEDVET | 3250 | 465 |
| v2.44 | NCEWSAFSCFQKAQLKSANTGNNERIINVSIKKLKRKPPS | | |
| | TNAGGGQGHELTCPSCDSYEKKPPKEFLERFKSLLQKMIH | | |
| | QHLSSRTHGSEDS | | |
| IL-21 | QGQDRHMIDMRQLIDIVDQLKNYVNDLVPEFLPAPEDVET | NB | 466 |
| v2.45 | NCEWSAFSCFQKAQLKSANTGNNERIINVSIKKLKRKPPS | | |
| | TNAGGGQGHELTCPSCDSYEKKPPKEFLERFKSLLQKMIH | | |
| | QHLSSRTHGSEDS | | |
| IL-21 | QGQDRHMIEMRQLIDIVDQLKNYVNDLVPEFLPAPEDVET | NB | 467 |
| v2.46 | NCEWSAFSCFQKAQLKSANTGNNERIINVSIKKLKRKPPS | | |
| | TNAGGGQGHELTCPSCDSYEKKPPKEFLERFKSLLQKMIH | | |
| | QHLSSRTHGSEDS | | |
| IL-21 | QGQDRHMIHMRQLIDIVDQLKNYVNDLVPEFLPAPEDVET | 16.5 | 468 |
| v2.47 | NCEWSAFSCFQKAQLKSANTGNNERIINVSIKKLKRKPPS | | |
| | TNAGGGQGHELTCPSCDSYEKKPPKEFLERFKSLLQKMIH | | |
| | QHLSSRTHGSEDS | | |
| IL-21 | QGQDRHMISMRQLIDIVDQLKNYVNDLVPEFLPAPEDVET | 4760 | 469 |
| v2.48 | NCEWSAFSCFQKAQLKSANTGNNERIINVSIKKLKRKPPS | | |
| | TNAGGGQGHELTCPSCDSYEKKPPKEFLERFKSLLQKMIH | | |
| | QHLSSRTHGSEDS | | |
| IL-21 | QGQDRHMITMRQLIDIVDQLKNYVNDLVPEFLPAPEDVET | >5000 | 470 |
| v2.49 | NCEWSAFSCFQKAQLKSANTGNNERIINVSIKKLKRKPPS | | |
| | TNAGGGQGHELTCPSCDSYEKKPPKEFLERFKSLLQKMIH | | |
| | QHLSSRTHGSEDS | | |
| IL-21 | QGQDRHMINMRQLIDIVDQLKNYVNDLVPEFLPAPEDVET | 2880 | 471 |
| v2.50 | NCEWSAFSCFQKAQLKSANTGNNERIINVSIKKLKRKPPS | | |
| | TNAGGGQGHELTCPSCDSYEKKPPKEFLERFKSLLQKMIH | | |
| | QHLSSRTHGSEDS | | |
| IL-21 | QGQDRHMIGMRQLIDIVDQLKNYVNDLVPEFLPAPEDVET | 4840 | 472 |
| v2.51 | NCEWSAFSCFQKAQLKSANTGNNERIINVSIKKLKRKPPS | | |
| | TNAGGGQGHELTCPSCDSYEKKPPKEFLERFKSLLQKMIH | | |
| | QHLSSRTHGSEDS | | |
| IL-21 | QGQDRHMIVMRQLIDIVDQLKNYVNDLVPEFLPAPEDVET | >5000 | 473 |
| v2.52 | NCEWSAFSCFQKAQLKSANTGNNERIINVSIKKLKRKPPS | | |
| | TNAGGGQGHELTCPSCDSYEKKPPKEFLERFKSLLQKMIH | | |
| | QHLSSRTHGSEDS | | |
| IL-21 | QGQDRHMIIMRQLIDIVDQLKNYVNDLVPEFLPAPEDVET | >5000 | 474 |
| v2.53 | NCEWSAFSCFQKAQLKSANTGNNERIINVSIKKLKRKPPS | | |
| | TNAGGGQGHELTCPSCDSYEKKPPKEFLERFKSLLQKMIH | | |
| | QHLSSRTHGSEDS | | |
| IL-21 | QGQDRHMILMRQLIDIVDQLKNYVNDLVPEFLPAPEDVET | 4690 | 475 |
| v2.54 | NCEWSAFSCFQKAQLKSANTGNNERIINVSIKKLKRKPPS | | |
| | TNAGGGQGHELTCPSCDSYEKKPPKEFLERFKSLLQKMIH | | |
| | QHLSSRTHGSEDS | | |
| IL-21 | QGQDRHMIYMRQLIDIVDQLKNYVNDLVPEFLPAPEDVET | 394 | 476 |
| v2.55 | NCEWSAFSCFQKAQLKSANTGNNERIINVSIKKLKRKPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLIDIVDQLKNYVNDLVPEFLPAPEDVET | 156 | 477 |
| v2.56 | NCEWSAFSCFQKAQLKSANTGNNERIINVSIAKLKFKPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLIDIVDQLKNYVNDLVPEFLPAPEDVET | 18.9 | 478 |
| v2.57 | NCEWSAFSCFQKAQLKSANTGNNERIINVSIKKLAFKPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLIDIVDQLKNYVNDLVPEFLPAPEDVET | 420 | 479 |
| v2.58 | NCEWSAFSCFQKAQLKSANTGNNERIINVSIKKLKFAPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLIDIVDQLKNYVNDLVPEFLPAPEDVET | 24.4 | 480 |
| v2.59 | NCEWSAFSCFQKAQLKSANTGNNERIINVSIKKLEFKPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLIDIIDQLKNYVNDLVPEFLPAPEDVET | 0.15 | 481 |
| v2.60 | NCEWSAFSCFQKAQLKSANTGNNERIINVSIKKIKRKPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLIDAVDQLKNYVNDLVPEFLPAPEDVET | 11.9 | 482 |
| v2.61 | NCEWSAFSCFQKAQLKSANTGNNERIINVSIKKEKRKPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLIDSIDQLKNYVNDLVPEFLPAPEDVET | 13.6 | 483 |
| v2.62 | NCEWSAFSCFQKAQLKSANTGNNERIINVSIKKVKRKPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLIDRIDQLKNYVNDLVPEFLPAPEDVET | 1400 | 484 |
| v2.63 | NCEWSAFSCFQKAQLKSANTGNNERIINVSIKKIKRKPPS | | |
| | TNAGGGQGHELTCPSCDSYEKKPPKEFLERFKSLLQKMIH | | |
| | QHLSSRTHGSEDS | | |
| IL-21 | QGQDRHMIRMRQFIDAADQLKNYVNDLVPEFLPAPEDVET | 1050 | 485 |
| v2.64 | NCEWSAFSCFQKAQLKSANTGNNERIINVSIKKMKRKPPS | | |
| | TNAGGGQGHELTCPSCDSYEKKPPKEFLERFKSLLQKMIH | | |
| | QHLSSRTHGSEDS | | |
| IL-21 | QGQDRHMIRMRQRIDAIDQLKNYVNDLVPEFLPAPEDVET | 5910 | 486 |
| v2.65 | NCEWSAFSCFQKAQLKSANTGNNERIINVSIKKIKRKPPS | | |
| | TNAGGGQGHELTCPSCDSYEKKPPKEFLERFKSLLQKMIH | | |
| | QHLSSRTHGSEDS | | |
| IL-21 | QGQDRHMIRMRQLIDIVDQLKNYVNDLVPEFLPAPEDVET | >10000 | 487 |
| v31.4 | NCEWSAFSCFQKAQLKSANTGNNERIINVSIKKLKEKPPG | | |
| | GEGGGGGGGEGGCPSCDSYEKKPPKEFLERFKSLLQKMIH | | |
| | QHLSSRTHGSEDS | | |
| IL-21 | QGQDRHMIRMRQLIDIVDQLKNYVNDLVPEFLPAPEDVET | — | 488 |
| v31.6 | NCEWSAFSCFQKAQLKSANTGNNERIINVSIKKLKFKPPG | | |
| | GEGGGGGGGEGGCPSCDSYEKKPPKEFLEREKSLLQKMIH | | |
| | QHLSSRTHGSEDS | | |
| IL-21 | QGQDRHMIRMRQLIDIVDQLKNYVNDLVPEFLPAPEDVET | 6059 | 489 |
| v31.23 | NCEWSAFSCFQKAQLKSANTGNNERIINVSIKKLKQKPPG | | |
| | GEGGGGGGGEGGCPSCDSYEKKPPKEFLERFKSLLQKMIH | | |
| | QHLSSRTHGSEDS | | |
| IL-21 | QGQDRHMISMRQLIDIVDQLKNYVNDLVPEFLPAPEDVET | — | 490 |
| v31.48 | NCEWSAFSCFQKAQLKSANTGNNERIINVSIKKLKRKPPG | | |
| | GEGGGGGGGEGGCPSCDSYEKKPPKEFLERFKSLLQKMIH | | |
| | QHLSSRTHGSEDS | | |
| IL-21 | QGQDRHMIGMRQLIDIVDQLKNYVNDLVPEFLPAPEDVET | 7730 | 491 |
| v31.51 | NCEWSAFSCFQKAQLKSANTGNNERIINVSIKKLKRKPPG | | |
| | GEGGGGGGGEGGCPSCDSYEKKPPKEFLERFKSLLQKMIH | | |
| | QHLSSRTHGSEDS | | |

ND = not detected

### IL-21 Fusion Protein Properties

In some embodiments, the fusion protein of the present disclosure provides selective activation of T cells, e.g., CD8+ T cells, over other non-CD8+ cell types, such as CD4+ T cells or NK cells. IL-21 generally acts on a broad class of cells, such as regulatory T cells. Stimulation and subsequent activation of cells via IL-21 can be measured by phosphorylation of STAT3. Cellular activation by IL-21 can be reported as an EC50 of STAT3 phosphorylation (pSTAT3). In some embodiments, the contacting results in a selective increase in cytotoxic function of CD8+ T cells in the culture of cells, as compared to the cytotoxic function of the CD8+ T cells, upon contacting the culture of cells with a reference construct that comprises the same IL-21 polypeptide but does not comprise the antigen binding protein or functional fragment thereof.

In some embodiments, potency of activation of CD8+ T cell and other cell types is measured by EC50 of cell activation as assessed by STAT3 phosphorylation. In some embodiments, the fusion protein has an EC50 of STAT3 phosphorylation for CD8+ T cells lower than other cell types. In some embodiments, the other cells are non-CD8+ cells. The non-CD8+ cells can be, but are not limited to B cells, CD4+ T cells, or NK cells. In some embodiments, the selective activation of CD8+ T cells is measured by an increase in STAT3 phosphorylation of the CD8+ T cells compared to the STAT3 phosphorylation of non-CD8+ cells in the culture of cells. In some embodiments, the increase in STAT3 phosphorylation is at least 5×, at least 10× or at least 100× more for the CD8+ T cells compared to the STAT3 phosphorylation of non-CD8+ cells in the culture of cells. In some embodiments, the targeted cytokine construct has a lower EC50 of STAT3 phosphorylation for CD8+ T cells than other cell types by at least 5×, at least 10×, at least 50×, at least 100×, at least 1,000×, at least 10,000×, or at least 100,000×.

In some embodiments, the fusion protein activates CD8+ T cells with at least about 10-fold or greater potency as compared to activation of NK cells. In some embodiments, the fusion protein activates CD8+ T cells a potency that is at least about 10-fold to about 50-fold greater as compared to activation of NK cells. In some embodiments, the fusion protein activates CD8+ T cells a potency that is at least about 10-fold to about 15-fold, 15-fold to about 20-fold, 20-fold to about 25-fold, 25-fold to about 30-fold, 30-fold to about 35-fold, 35-fold to about 40-fold, 40-fold to about 45-fold, 45-fold to about 50-fold, 50-fold to about 100-fold, 100-fold to about 1,000-fold, 1,000-fold to about 10,000-fold, 10,000-fold to about 100,000-fold, about 10-fold, 15-fold, 20-fold, 25-fold, 30-fold, 35-fold, 40-fold, 45-fold, 50-fold, 100-fold, 1,000-fold, 10,000-fold or about 100,000-fold greater as compared to activation of NK cells.

In some embodiments, the fusion protein activates CD8+ T cells with at least about 10-fold or greater potency as compared to activation of CD4+ T cells. In some embodiments, the fusion protein activates CD8+ T cells a potency that is at least about 10-fold to about 50-fold greater as compared to activation of CD4+ cells. In some embodiments, the fusion protein activates CD4+ T cells a potency that is at least about 10-fold to about 15-fold, 15-fold to about 20-fold, 20-fold to about 25-fold, 25-fold to about 30-fold, 30-fold to about 35-fold, 35-fold to about 40-fold, 40-fold to about 45-fold, 45-fold to about 50-fold, 50-fold to about 100-fold, 100-fold to about 1,000-fold, 1,000-fold to about 10,000-fold, 10,000-fold to about 100,000-fold, about 10-fold, 15-fold, 20-fold, 25-fold, 30-fold, 35-fold, 40-fold, 45-fold, 50-fold, 100-fold, 1,000-fold, 10,000-fold or about 100,000-fold greater as compared to activation of CD4+ T cells.

### Exemplary IL-21 Fusion Proteins

In some embodiments, the disclosure provides a fusion protein comprising an anti-CD8 antibody or antigen binding fragment thereof described herein, and an IL-21 polypeptide described herein.

In some embodiments, the disclosure provides a fusion protein comprising:
a) an IL-21 polypeptide comprising (i) one or more amino acid substitutions providing a reduced isoelectric point relative to a human IL-21 polypeptide comprising SEQ ID NO: 390, wherein the one or more amino acid substitutions are within S80 to T92 of SEQ ID NO: 39, wherein at least one amino acid substitution is at residue R85, R86, K88, H89, R90, or a combination thereof, and (ii) at least one amino acid substitution providing reduced binding to a human IL-21 receptor relative to binding by the human IL-21 polypeptide; and
b) an antibody or an antigen binding fragment thereof that specifically binds to CD8αβ or CD8β as described herein.

In some embodiments, the disclosure provides a fusion protein comprising:
a) an IL-21 polypeptide comprising (i) four or more amino acid substitutions providing a reduced isoelectric point relative to a human IL-21 polypeptide comprising SEQ ID NO: 390, wherein the four or more amino acid substitutions are within S80 to T92 of SEQ ID NO: 390, wherein at least four amino acid substitutions are at residues R85, R86, K88, H89, R90, or a combination thereof, and (ii) at least one amino acid substitution providing reduced binding to a human IL-21 receptor relative to binding by the human IL-21 polypeptide; and
b) an antibody or an antigen binding fragment thereof that specifically binds to CD8αβ or CD8β as described herein.

In some embodiments, the disclosure provides a fusion protein comprising:
a) an IL-21 polypeptide comprising (i) four or more amino acid substitutions providing a reduced isoelectric point relative to a human IL-21 polypeptide comprising SEQ ID NO: 390, wherein the four or more amino acid substitutions are within S80 to T92 of SEQ ID NO: 390, wherein at least four amino acid substitutions are at residues R85, R86, K88, H89, R90, or a combination thereof, and (ii) at least one amino acid substitution providing reduced binding to a human IL-21 receptor relative to binding by the human IL-21 polypeptide, wherein the at least one amino acid substitution is at a residue selected from R5, I8, R9, RI1, L13, I14, I16, V17, D18, K72, K73, L74, K75, R76, K77, K117, and a combination thereof; and
b) an antibody or an antigen binding fragment thereof that specifically binds to CD8αβ or CD8β as described herein.

In some embodiments, the disclosure provides a fusion protein comprising:
a) an IL-21 polypeptide comprising (i) four or more amino acid substitutions providing a reduced isoelectric point relative to a human IL-21 polypeptide comprising SEQ ID NO: 390, wherein the four or more amino acid substitutions are within S80 to T92 of SEQ ID NO: 390, wherein at least four amino acid substitutions are at residues R85, R86, K88, H89, R90, or a combination thereof, and (ii) at least one amino acid substitution selected from: (a) RI1D; (b) RI1E; (c) I14D, D18A and K117A; (d) R76E; (e) R5F; (f) R76F; (g) 18E; (h) R5A; (i) R5E; (j) R5S; (k) R5T; (1) R5N; (m) R5Q; (n) R5V; (o) R5I; (p) R5L; (q) R5Y; (r) R76A; (s) R76N; (t) R76D; (u) R76S; (v) R76T; (w) R76Q; (x) R76V; (y) R76I; (z) R76L; (aa) R76Y; (bb) K77A; (cc) K77E; (dd) K72A; (ee) K72E; (ff) K75A; (gg) K75E; (hh) K73A; (ii) K73E; (jj) R5F and K77A; (kk) R5F and K77E; (11) R5F and K72A; (mm) R5F and K72E; (nn) R5F and K76A; (oo) R5F and K76E; (pp) K73A and K76F; (qq) K73E and K76F; (rr) R9A; (ss) R9D; (tt) R9E; (uu) R9H; (vv) R9S; (ww) R9T; (xx) R9N; (zz) R9G; (aaa) R9V; (bbb) R9I; (ccc) R9L; (ddd) R9Y; (eee) K72A and R76F; (fff) K75A and R76F; (ggg) R76F and K77A; (hhh) K75E and R76F; (iii) V171 and L741; (jjj) 116A and L74F; (kkk) 116S, V171, and L74V; (111) 116R, V171, and L74I; (mmm) L13F, 116A, V17A and L74M; and (nnn) L13R, 116A, V171 and L741; and
b) an antibody or an antigen binding fragment thereof that specifically binds to CD8αβ or CD8β as described herein.

In some embodiments, the disclosure provides a fusion protein comprising:
a) an IL-21 polypeptide comprising (i) four or more amino acid substitutions providing a reduced isoelectric point relative to a human IL-21 polypeptide comprising SEQ ID NO: 390, wherein the four or more amino acid substitutions are at residues S80, T81, N82, A83, R85, R86, Q87, K88, H89, R90, L91, T92, or a combination thereof, and (ii) at least one amino acid substitution providing reduced binding to a human IL-21 receptor relative to binding by the human IL-21 polypeptide, wherein the at least one amino acid substitution is at a residue selected from R5, I8, R9, R11, L13, I14, I16, V17, D18, K72, K73, L74, K75, R76, K77, K117, and a combination thereof; and
b) an antibody or an antigen binding fragment thereof that specifically binds to CD8αβ or CD8β as described herein.

In some embodiments, the disclosure provides a fusion protein comprising:
a) an IL-21 polypeptide comprising (i) four or more amino acid substitutions providing a reduced isoelectric point relative to a human IL-21 polypeptide comprising SEQ ID NO: 390, wherein the four or more amino acid substitutions are at residues S80, T81, N82, A83, R85, R86, Q87, K88, H89, R90, L91, T92, or a combination thereof, and (ii) at least one amino acid substitution providing reduced binding to a human IL-21 receptor relative to binding by the human IL-21 polypeptide, wherein the at least one amino acid substitution is at residue R76; and
b) an antibody or an antigen binding fragment thereof that specifically binds to CD8αβ or CD8β as described herein.

In some embodiments, the disclosure provides a fusion protein comprising:
a) an IL-21 polypeptide comprising (i) an amino acid sequence selected from SEQ ID NOs: 391-421, and (ii) at least one amino acid substitution providing reduced binding to a human IL-21 receptor relative to binding by the human IL-21 polypeptide; and b) an antibody or an antigen binding fragment thereof that specifically binds to CD8αβ or CD8β as described herein.

In some embodiments, the disclosure provides a fusion protein comprising:
a) an IL-21 polypeptide comprising (i) an amino acid sequence selected from SEQ ID NOs: 391-421, and (ii) at least one amino acid substitution providing reduced binding to a human IL-21 receptor relative to binding by the human IL-21 polypeptide, wherein the at least one amino acid substitution is at a residue selected from R5, I8, R9, R11, L13, I14, I16, V17, D18, K72, K73, L74, K75, R76, K77, K117, and a combination thereof; and
b) an antibody or an antigen binding fragment thereof that specifically binds to CD8αβ or CD8β as described herein.

In some embodiments, the disclosure provides a fusion protein comprising:
a) an IL-21 polypeptide comprising (i) an amino acid sequence selected from SEQ ID NOs: 391-421, and (ii) at least one amino acid substitution providing reduced binding to a human IL-21 receptor relative to binding by the human IL-21 polypeptide, wherein the at least one amino acid substitution is at residue R76; and
b) an antibody or an antigen binding fragment thereof that specifically binds to CD8αβ or CD8β as described herein.

In some embodiments, the disclosure provides a fusion protein comprising:
a) an IL-21 polypeptide comprising (i) four or more amino acid substitutions providing a reduced isoelectric point relative to a human IL-21 polypeptide comprising SEQ ID NO: 390, wherein the four or more amino acid substitutions are within S80 to T92 of SEQ ID NO: 390, wherein at least four amino acid substitutions are at residues R85, R86, K88, H89, R90, or a combination thereof, and (ii) at least one amino acid substitution providing reduced binding to a human IL-21 receptor relative to binding by the human IL-21 polypeptide; and
b) an antibody or an antigen binding fragment thereof that specifically binds to CD8αβ or CD8β, wherein the antibody or antigen binding fragment thereof comprises:
   (i) a VH domain comprising a CDR-H1 comprising the amino acid sequence of SEQ ID NO: 229, a CDR-H2 comprising the amino acid sequence of SEQ ID NO: 230, and a CDR-H3 comprising the amino acid sequence of SEQ ID NO: 231; and a VL domain comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
   (ii) a VH domain comprising a CDR-H1 comprising the amino acid sequence of SEQ ID NO: 225, a CDR-H2 comprising the amino acid sequence of SEQ ID NO: 232, and a CDR-H3 comprising the amino acid sequence of SEQ ID NO: 233; and a VL domain comprising a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236;
   (iii) a VH domain comprising a CDR-H1 comprising the amino acid sequence of SEQ ID NO: 240, a CDR-H2 comprising the amino acid sequence of SEQ ID NO: 241, and a CDR-H3 comprising the amino acid sequence of SEQ ID NO: 242; and a VL domain comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42; or
   (iv) a VH domain comprising a CDR-H1 comprising the amino acid sequence of SEQ ID NO: 238, a CDR-H2 comprising the amino acid sequence of SEQ ID NO: 243, and a CDR-H3 comprising the amino acid sequence of SEQ ID NO: 233; and a VL domain comprising a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236.

In some embodiments, the disclosure provides a fusion protein comprising:
a) an IL-21 polypeptide comprising (i) four or more amino acid substitutions providing a reduced isoelectric point relative to a human IL-21 polypeptide comprising SEQ ID NO: 390, wherein the four or more amino acid substitutions are within S80 to T92 of SEQ ID NO: 390, wherein at least four amino acid substitutions are at residues R85, R86, K88, H89, R90, or a combination thereof, and (ii) at least one amino acid substitution providing reduced binding to a human IL-21 receptor relative to binding by the human IL-21 polypeptide, wherein the at least one amino acid substitution is at a residue selected from R5, 18, R9, RI1, L13, I14, I16, V17, D18, K72, K73, L74, K75, R76, K77, K117, and a combination thereof; and
b) an antibody or an antigen binding fragment thereof that specifically binds to CD8αβ or CD8β, wherein the antibody or antigen binding fragment thereof comprises:
   (i) a VH domain comprising a CDR-H1 comprising the amino acid sequence of SEQ ID NO: 229, a CDR-H2 comprising the amino acid sequence of SEQ ID NO: 230, and a CDR-H3 comprising the amino acid sequence of SEQ ID NO: 231; and a VL domain comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
   (ii) a VH domain comprising a CDR-H1 comprising the amino acid sequence of SEQ ID NO: 225, a CDR-H2 comprising the amino acid sequence of SEQ ID NO: 232, and a CDR-H3 comprising the amino acid sequence of SEQ ID NO: 233; and a VL domain comprising a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236;
   (iii) a VH domain comprising a CDR-H1 comprising the amino acid sequence of SEQ ID NO: 240, a CDR-H2 comprising the amino acid sequence of SEQ ID NO: 241, and a CDR-H3 comprising the amino acid sequence of SEQ ID NO: 242; and a VL domain comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42; or
   (iv) a VH domain comprising a CDR-H1 comprising the amino acid sequence of SEQ ID NO: 238, a CDR-H2 comprising the amino acid sequence of SEQ ID NO: 243, and a CDR-H3 comprising the amino acid sequence of SEQ ID NO: 233; and a VL domain comprising a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236.

In some embodiments, the disclosure provides a fusion protein comprising:
a) an IL-21 polypeptide comprising (i) four or more amino acid substitutions providing a reduced isoelectric point relative to a human IL-21 polypeptide comprising SEQ ID NO: 390, wherein the four or more amino acid substitutions are within S80 to T92 of SEQ ID NO: 390, wherein at least four amino acid substitutions are at residues R85, R86, K88, H89, R90, or a combination thereof, and (ii) at least one amino acid substitution selected from: (a) RI1D; (b) RI1E; (c) 114D, D18A and K117A; (d) R76E; (e) R5F; (f) R76F; (g) 18E; (h) R5A; (i) R5E; (j) R5S; (k) R5T; (1) R5N; (m) R5Q; (n) R5V; (o) R51; (p) R5L; (q) R5Y; (r) R76A; (s) R76N; (t) R76D; (u) R76S; (v) R76T; (w) R76Q; (x) R76V; (y) R761; (z) R76L; (aa) R76Y; (bb) K77A; (cc) K77E; (dd) K72A; (ee) K72E; (ff) K75A; (gg) K75E; (hh) K73A; (ii) K73E; (jj) R5F and K77A; (kk) R5F and K77E; (11) R5F and K72A; (mm) R5F and K72E; (nn) R5F and K76A; (oo) R5F and K76E; (pp) K73A and K76F; (qq) K73E and K76F; (rr) R9A; (ss) R9D; (tt) R9E; (uu) R9H; (vv) R9S; (ww) R9T; (xx) R9N; (zz) R9G; (aaa) R9V; (bbb) R91; (ccc) R9L; (ddd) R9Y; (eee) K72A and R76F; (fff) K75A and R76F; (ggg) R76F and K77A; (hhh) K75E and R76F; (iii) V171 and L741; (jjj) 116A and L74F; (kkk) 116S, V171, and L74V; (111) 116R, V171, and L741; (mmm) L13F, I16A, V17A and L74M; and (nnn) L13R, 116A, V171 and L741; and
b) an antibody or an antigen binding fragment thereof that specifically binds to CD8αβ or CD8β, wherein the antibody or antigen binding fragment thereof comprises:
   (i) a VH domain comprising a CDR-H1 comprising the amino acid sequence of SEQ ID NO: 229, a CDR-H2 comprising the amino acid sequence of SEQ ID NO: 230, and a CDR-H3 comprising the amino acid sequence of SEQ ID NO: 231; and a VL domain comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
   (ii) a VH domain comprising a CDR-H1 comprising the amino acid sequence of SEQ ID NO: 225, a CDR-H2 comprising the amino acid sequence of SEQ ID NO: 232, and a CDR-H3 comprising the amino acid sequence of SEQ ID NO: 233; and a VL domain comprising a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236;
   (iii) a VH domain comprising a CDR-H1 comprising the amino acid sequence of SEQ ID NO: 240, a CDR-H2 comprising the amino acid sequence of SEQ ID NO: 241, and a CDR-H3 comprising the amino acid sequence of SEQ ID NO: 242; and a VL domain comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42; or
   (iv) a VH domain comprising a CDR-H1 comprising the amino acid sequence of SEQ ID NO: 238, a CDR-H2 comprising the amino acid sequence of SEQ ID NO: 243, and a CDR-H3 comprising the amino acid sequence of SEQ ID NO: 233; and a VL domain comprising a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236.

In some embodiments, the disclosure provides a fusion protein comprising:
a) an IL-21 polypeptide comprising (i) four or more amino acid substitutions providing a reduced isoelectric point relative to a human IL-21 polypeptide comprising SEQ ID NO: 390, wherein the four or more amino acid substitutions are at residues S80, T81, N82, A83, R85, R86, Q87, K88, H89, R90, L91, T92, or a combination thereof, and (ii) at least one amino acid substitution providing reduced binding to a human IL-21 receptor relative to binding by the human IL-21 polypeptide, wherein the at least one amino acid substitution is at a residue selected from R5, I8, R9, R11, L13, I14, I16, V17, D18, K72, K73, L74, K75, R76, K77, K117, and a combination thereof; and
b) an antibody or an antigen binding fragment thereof that specifically binds to CD8αβ or CD8β, wherein the antibody or antigen binding fragment thereof comprises:
   (i) a VH domain comprising a CDR-H1 comprising the amino acid sequence of SEQ ID NO: 229, a CDR-H2 comprising the amino acid sequence of SEQ ID NO: 230, and a CDR-H3 comprising the amino acid sequence of SEQ ID NO: 231; and a VL domain comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
   (ii) a VH domain comprising a CDR-H1 comprising the amino acid sequence of SEQ ID NO: 225, a CDR-H2 comprising the amino acid sequence of SEQ ID NO: 232, and a CDR-H3 comprising the amino acid sequence of SEQ ID NO: 233; and a VL domain comprising a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236;
   (iii) a VH domain comprising a CDR-H1 comprising the amino acid sequence of SEQ ID NO: 240, a CDR-H2 comprising the amino acid sequence of SEQ ID NO: 241, and a CDR-H3 comprising the amino acid sequence of SEQ ID NO: 242; and a VL domain comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42; or
   (iv) a VH domain comprising a CDR-H1 comprising the amino acid sequence of SEQ ID NO: 238, a CDR-H2 comprising the amino acid sequence of SEQ ID NO: 243, and a CDR-H3 comprising the amino acid sequence of SEQ ID NO: 233; and a VL domain comprising a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236.

In some embodiments, the disclosure provides a fusion protein comprising:
a) an IL-21 polypeptide comprising (i) four or more amino acid substitutions providing a reduced isoelectric point relative to a human IL-21 polypeptide comprising SEQ ID NO: 390, wherein the four or more amino acid substitutions are at residues S80, T81, N82, A83, R85, R86, Q87, K88, H89, R90, L91, T92, or a combination thereof, and (ii) at least one amino acid substitution providing reduced binding to a human IL-21 receptor relative to binding by the human IL-21 polypeptide, wherein the at least one amino acid substitution is at residue R76; and
b) an antibody or an antigen binding fragment thereof that specifically binds to CD8αβ or CD8β, wherein the antibody or antigen binding fragment thereof comprises:
   (i) a VH domain comprising a CDR-H1 comprising the amino acid sequence of SEQ ID NO: 229, a CDR-H2 comprising the amino acid sequence of SEQ ID NO: 230, and a CDR-H3 comprising the amino acid sequence of SEQ ID NO: 231; and a VL domain comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
   (ii) a VH domain comprising a CDR-H1 comprising the amino acid sequence of SEQ ID NO: 225, a CDR-H2 comprising the amino acid sequence of SEQ ID NO: 232, and a CDR-H3 comprising the amino acid sequence of SEQ ID NO: 233; and a VL domain comprising a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236;
   (iii) a VH domain comprising a CDR-H1 comprising the amino acid sequence of SEQ ID NO: 240, a CDR-H2 comprising the amino acid sequence of SEQ ID NO: 241, and a CDR-H3 comprising the amino acid sequence of SEQ ID NO: 242; and a VL domain comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42; or
   (iv) a VH domain comprising a CDR-H1 comprising the amino acid sequence of SEQ ID NO: 238, a CDR-H2 comprising the amino acid sequence of SEQ ID NO: 243, and a CDR-H3 comprising the amino acid sequence of SEQ ID NO: 233; and a VL domain comprising a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236.

In some embodiments, the disclosure provides a fusion protein comprising:
a) an IL-21 polypeptide comprising (i) an amino acid sequence selected from SEQ ID NOs: 391-421, and (ii) at least one amino acid substitution providing reduced binding to a human IL-21 receptor relative to binding by the human IL-21 polypeptide; and
b) an antibody or an antigen binding fragment thereof that specifically binds to CD8αβ or CD8β, wherein the antibody or antigen binding fragment thereof comprises:
   (i) a VH domain comprising a CDR-H1 comprising the amino acid sequence of SEQ ID NO: 229, a CDR-H2 comprising the amino acid sequence of SEQ ID NO: 230, and a CDR-H3 comprising the amino acid sequence of SEQ ID NO: 231; and a VL domain comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
   (ii) a VH domain comprising a CDR-H1 comprising the amino acid sequence of SEQ ID NO: 225, a CDR-H2 comprising the amino acid sequence of SEQ ID NO: 232, and a CDR-H3 comprising the amino acid sequence of SEQ ID NO: 233; and a VL domain comprising a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236;
   (iii) a VH domain comprising a CDR-H1 comprising the amino acid sequence of SEQ ID NO: 240, a CDR-H2 comprising the amino acid sequence of SEQ ID NO: 241, and a CDR-H3 comprising the amino acid sequence of SEQ ID NO: 242; and a VL domain comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42; or
   (iv) a VH domain comprising a CDR-H1 comprising the amino acid sequence of SEQ ID NO: 238, a CDR-H2 comprising the amino acid sequence of SEQ ID NO: 243, and a CDR-H3 comprising the amino acid sequence of SEQ ID NO: 233; and a VL domain comprising a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236.

In some embodiments, the disclosure provides a fusion protein comprising:
a) an IL-21 polypeptide comprising (i) an amino acid sequence selected from SEQ ID NOs: 391-421, and (ii) at least one amino acid substitution providing reduced binding to a human IL-21 receptor relative to binding by the human IL-21 polypeptide, wherein the at least one amino acid substitution is at a residue selected from R5, I8, R9, R11, L13, I14, I16, V17, D18, K72, K73, L74, K75, R76, K77, K117, and a combination thereof; and
b) an antibody or an antigen binding fragment thereof that specifically binds to CD8αβ or CD8β, wherein the antibody or antigen binding fragment thereof comprises:
   (i) a VH domain comprising a CDR-H1 comprising the amino acid sequence of SEQ ID NO: 229, a CDR-H2 comprising the amino acid sequence of SEQ ID NO: 230, and a CDR-H3 comprising the amino acid sequence of SEQ ID NO: 231; and a VL domain comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
   (ii) a VH domain comprising a CDR-H1 comprising the amino acid sequence of SEQ ID NO: 225, a CDR-H2 comprising the amino acid sequence of SEQ ID NO: 232, and a CDR-H3 comprising the amino acid sequence of SEQ ID NO: 233; and a VL domain comprising a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236;
   (iii) a VH domain comprising a CDR-H1 comprising the amino acid sequence of SEQ ID NO: 240, a CDR-H2 comprising the amino acid sequence of SEQ ID NO: 241, and a CDR-H3 comprising the amino acid sequence of SEQ ID NO: 242; and a VL domain comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42; or
   (iv) a VH domain comprising a CDR-H1 comprising the amino acid sequence of SEQ ID NO: 238, a CDR-H2 comprising the amino acid sequence of SEQ ID NO: 243, and a CDR-H3 comprising the amino acid sequence of SEQ ID NO: 233; and a VL domain comprising a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236.

In some embodiments, the disclosure provides a fusion protein comprising:
a) an IL-21 polypeptide comprising (i) an amino acid sequence selected from SEQ ID NOs: 391-421, and (ii) at least one amino acid substitution providing reduced binding to a human IL-21 receptor relative to binding by the human IL-21 polypeptide, wherein the at least one amino acid substitution is at residue R76; and
b) an antibody or an antigen binding fragment thereof that specifically binds to CD8αβ or CD8β, wherein the antibody or antigen binding fragment thereof comprises:
   (i) a VH domain comprising a CDR-H1 comprising the amino acid sequence of SEQ ID NO: 229, a CDR-H2 comprising the amino acid sequence of SEQ ID NO: 230, and a CDR-H3 comprising the amino acid sequence of SEQ ID NO: 231; and a VL domain comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
   (ii) a VH domain comprising a CDR-H1 comprising the amino acid sequence of SEQ ID NO: 225, a CDR-H2 comprising the amino acid sequence of SEQ ID NO: 232, and a CDR-H3 comprising the amino acid sequence of SEQ ID NO: 233; and a VL domain comprising a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236;
   (iii) a VH domain comprising a CDR-H1 comprising the amino acid sequence of SEQ ID NO: 240, a CDR-H2 comprising the amino acid sequence of SEQ ID NO: 241, and a CDR-H3 comprising the amino acid sequence of SEQ ID NO: 242; and a VL domain comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42; or
   (iv) a VH domain comprising a CDR-H1 comprising the amino acid sequence of SEQ ID NO: 238, a CDR-H2 comprising the amino acid sequence of SEQ ID NO: 243, and a CDR-H3 comprising the amino acid sequence of SEQ ID NO: 233; and a VL domain comprising a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236.

In some embodiments, the fusion protein comprises one, two, or all three polypeptides shown for a single fusion protein in Table 10.

In some embodiments, a fusion protein of the present disclosure comprises two light chains, a heavy chain comprising an IL-21 fusion, and a heavy chain not comprising an IL-21 fusion for a single fusion protein in Table 10. As is known in the art, the C-terminal lysine of some antibody heavy chain species may be cleaved off in some fraction of molecules. In some embodiments, a fusion protein of the present disclosure comprises two light chains, a heavy chain comprising an IL-21 fusion, and a heavy chain not comprising an IL-21 fusion for a single fusion protein in Table 10, wherein the heavy chain not comprising the IL-21 fusion comprises the sequence of SEQ ID Nos: 559, 563, 567, 571, 575, 579, 583, 587 or 589 for the respective fusion protein. In some embodiments, a fusion protein of the present disclosure comprises two light chains, a heavy chain comprising an IL-21 fusion, and a heavy chain not comprising an IL-21 fusion for a single fusion protein in Table 10, wherein the heavy chain not comprising the IL-21 fusion comprises the sequence of SEQ ID Nos: 560, 564, 568, 572, 576, 580, 584, 588, or 592 for the respective fusion protein. In some embodiments, the present disclosure provides a plurality of fusion proteins of the present disclosure (e.g., in a mixture), wherein each fusion protein of the plurality comprises two light chains, a heavy chain comprising an IL-21 fusion, and a heavy chain not comprising an IL-21 fusion for a single fusion protein in Table 10, wherein the heavy chain not comprising the IL-21 fusion comprises the sequence of SEQ ID Nos: 559, 563, 567, 571, 575, 579, 583, 587 or 589 for the respective fusion protein, or the sequence of SEQ ID Nos: 560, 564, 568, 572, 576, 580, 584, 588, or 592 for the respective fusion protein, or the plurality comprises a mixture of species representing cleaved (e.g., comprising the sequence of SEQ ID Nos: 559, 563, 567, 571, 575, 579, 583, 587 or 589 for the respective fusion protein) and uncleaved (e.g., comprising the sequence of SEQ ID Nos: 560, 564, 568, 572, 576, 580, 584, 588, or 592 for the respective fusion protein) species.

In some embodiments, the present disclosure provides a fusion protein comprising two heavy chain sequences and two light chain sequences of a single fusion protein listed in Table 10, wherein one of the heavy chain sequences has an IL21 fusion and the other heavy chain sequence is without an IL21 fusion, and wherein the two light chain sequences are identical. In some embodiments, the heavy chain sequence without an IL21 fusion comprises a lysine at the C terminus.

In some embodiments, a fusion protein of the present disclosure comprises one or two light chains comprising the amino acid sequence of SEQ ID NO: 557, a heavy chain comprising the amino acid sequence of SEQ ID NO: 558 and a heavy chain comprising the amino acid sequence of SEQ ID NO: 559.

In some embodiments, a fusion protein of the present disclosure comprises one or two light chains comprising the amino acid sequence of SEQ ID NO: 561, a heavy chain comprising the amino acid sequence of SEQ ID NO: 562 and a heavy chain comprising the amino acid sequence of SEQ ID NO: 563.

In some embodiments, a fusion protein of the present disclosure comprises one or two light chains comprising the amino acid sequence of SEQ ID NO: 565, a heavy chain comprising the amino acid sequence of SEQ ID NO: 566 and a heavy chain comprising the amino acid sequence of SEQ ID NO: 567.

In some embodiments, a fusion protein of the present disclosure comprises one or two light chains comprising the amino acid sequence of SEQ ID NO: 569, a heavy chain comprising the amino acid sequence of SEQ ID NO: 570 and a heavy chain comprising the amino acid sequence of SEQ ID NO: 571.

In some embodiments, a fusion protein of the present disclosure comprises one or two light chains comprising the amino acid sequence of SEQ ID NO: 573, a heavy chain comprising the amino acid sequence of SEQ ID NO: 574 and a heavy chain comprising the amino acid sequence of SEQ ID NO: 575.

In some embodiments, a fusion protein of the present disclosure comprises one or two light chains comprising the amino acid sequence of SEQ ID NO: 577, a heavy chain comprising the amino acid sequence of SEQ ID NO: 578 and a heavy chain comprising the amino acid sequence of SEQ ID NO: 579.

In some embodiments, a fusion protein of the present disclosure comprises one or two light chains comprising the amino acid sequence of SEQ ID NO: 581, a heavy chain comprising the amino acid sequence of SEQ ID NO: 582 and a heavy chain comprising the amino acid sequence of SEQ ID NO: 583.

In some embodiments, a fusion protein of the present disclosure comprises one or two light chains comprising the amino acid sequence of SEQ ID NO: 585, a heavy chain comprising the amino acid sequence of SEQ ID NO: 586 and a heavy chain comprising the amino acid sequence of SEQ ID NO: 587.

In some embodiments, a fusion protein of the present disclosure comprises one or two light chains comprising the amino acid sequence of SEQ ID NO: 589, a heavy chain comprising the amino acid sequence of SEQ ID NO: 590 and a heavy chain comprising the amino acid sequence of SEQ ID NO: 591.

In some embodiments, a fusion protein of the present disclosure comprises one or two light chains comprising the amino acid sequence of SEQ ID NO: 557, a heavy chain comprising the amino acid sequence of SEQ ID NO: 558 and a heavy chain comprising the amino acid sequence of SEQ ID NO: 560.

In some embodiments, a fusion protein of the present disclosure comprises one or two light chains comprising the amino acid sequence of SEQ ID NO: 561, a heavy chain comprising the amino acid sequence of SEQ ID NO: 562 and a heavy chain comprising the amino acid sequence of SEQ ID NO: 564.

In some embodiments, a fusion protein of the present disclosure comprises one or two light chains comprising the amino acid sequence of SEQ ID NO: 565, a heavy chain comprising the amino acid sequence of SEQ ID NO: 566 and a heavy chain comprising the amino acid sequence of SEQ ID NO: 568.

In some embodiments, a fusion protein of the present disclosure comprises one or two light chains comprising the amino acid sequence of SEQ ID NO: 569, a heavy chain comprising the amino acid sequence of SEQ ID NO: 570 and a heavy chain comprising the amino acid sequence of SEQ ID NO: 572.

In some embodiments, a fusion protein of the present disclosure comprises one or two light chains comprising the amino acid sequence of SEQ ID NO: 573, a heavy chain comprising the amino acid sequence of SEQ ID NO: 574 and a heavy chain comprising the amino acid sequence of SEQ ID NO: 576.

In some embodiments, a fusion protein of the present disclosure comprises one or two light chains comprising the amino acid sequence of SEQ ID NO: 577, a heavy chain comprising the amino acid sequence of SEQ ID NO: 578 and a heavy chain comprising the amino acid sequence of SEQ ID NO: 580.

In some embodiments, a fusion protein of the present disclosure comprises one or two light chains comprising the amino acid sequence of SEQ ID NO: 581, a heavy chain comprising the amino acid sequence of SEQ ID NO: 582 and a heavy chain comprising the amino acid sequence of SEQ ID NO: 584.

In some embodiments, a fusion protein of the present disclosure comprises one or two light chains comprising the amino acid sequence of SEQ ID NO: 585, a heavy chain comprising the amino acid sequence of SEQ ID NO: 586 and a heavy chain comprising the amino acid sequence of SEQ ID NO: 588.

In some embodiments, a fusion protein of the present disclosure comprises one or two light chains comprising the amino acid sequence of SEQ ID NO: 589, a heavy chain comprising the amino acid sequence of SEQ ID NO: 590 and a heavy chain comprising the amino acid sequence of SEQ ID NO: 592.

In some embodiments, a fusion protein of the present disclosure comprises one or two light chains comprising the amino acid sequence of SEQ ID NO: 557, a heavy chain comprising the amino acid sequence of SEQ ID NO: 558 and a heavy chain comprising the amino acid sequence of SEQ ID NO: 559 or SEQ ID NO: 560.

In some embodiments, a fusion protein of the present disclosure comprises one or two light chains comprising the amino acid sequence of SEQ ID NO: 561, a heavy chain comprising the amino acid sequence of SEQ ID NO: 562 and a heavy chain comprising the amino acid sequence of SEQ ID NO: 563 or SEQ ID NO: 564.

In some embodiments, a fusion protein of the present disclosure comprises one or two light chains comprising the amino acid sequence of SEQ ID NO: 565, a heavy chain comprising the amino acid sequence of SEQ ID NO: 566 and a heavy chain comprising the amino acid sequence of SEQ ID NO: 567 or SEQ ID NO: 568.

In some embodiments, a fusion protein of the present disclosure comprises one or two light chains comprising the amino acid sequence of SEQ ID NO: 569, a heavy chain comprising the amino acid sequence of SEQ ID NO: 570 and a heavy chain comprising the amino acid sequence of SEQ ID NO: 571 or SEQ ID NO: 572.

In some embodiments, a fusion protein of the present disclosure comprises one or two light chains comprising the amino acid sequence of SEQ ID NO: 573, a heavy chain comprising the amino acid sequence of SEQ ID NO: 574 and a heavy chain comprising the amino acid sequence of SEQ ID NO: 575 or SEQ ID NO: 576.

In some embodiments, a fusion protein of the present disclosure comprises one or two light chains comprising the amino acid sequence of SEQ ID NO: 577, a heavy chain comprising the amino acid sequence of SEQ ID NO: 578 and a heavy chain comprising the amino acid sequence of SEQ ID NO: 579 or SEQ ID NO: 580.

In some embodiments, a fusion protein of the present disclosure comprises one or two light chains comprising the amino acid sequence of SEQ ID NO: 581, a heavy chain comprising the amino acid sequence of SEQ ID NO: 582 and a heavy chain comprising the amino acid sequence of SEQ ID NO: 583 or SEQ ID NO: 584.

In some embodiments, a fusion protein of the present disclosure comprises one or two light chains comprising the amino acid sequence of SEQ ID NO: 585, a heavy chain comprising the amino acid sequence of SEQ ID NO: 586 and a heavy chain comprising the amino acid sequence of SEQ ID NO: 587 or SEQ ID NO: 588.

In some embodiments, a fusion protein of the present disclosure comprises one or two light chains comprising the amino acid sequence of SEQ ID NO: 589, a heavy chain comprising the amino acid sequence of SEQ ID NO: 590 and a heavy chain comprising the amino acid sequence of SEQ ID NO: 591 or SEQ ID NO: 592.

TABLE 10
Exemplary IL-21:anti-CD8 Fusion Proteins

| | | | | Heavy chain |
|---|---|---|---|---|
| | | Heavy chain | Heavy chain | sequence |
| | | sequence | sequence | (without IL-21 |
| | Light chain | (with IL-21 | (without IL-21 | fusion) plus C- |
| Name | sequence | fusion) | fusion) | term lysine |
| xhCD8.1- | DIQMTQSPSSLS | QVQLVQSGAEVKKPGSSV | QVQLVQSGAEVKKPG | QVQLVQSGAEVKKPG |
| hIL21 | ASVGDRVTITCR | KVSCKASGGTFSSYAISW | SSVKVSCKASGGTFS | SSVKVSCKASGGTES |
| v31.4 | ASQSIYGALNWY | VRQAPGQGLEWMGGIIPG | SYAISWVRQAPGQGL | SYAISWVRQAPGQGL |
| | QQKPGKAPKLLI | YATANYAQKFQGRVTITA | EWMGGIIPGYATANY | EWMGGIIPGYATANY |
| | YGASNLQSGVPS | DESTSTAYMELSSLRSED | AQKFQGRVTITADES | AQKFQGRVTITADES |
| | RFSGSGSGTDET | TAVYYCARDAAGIRLFAD | TSTAYMELSSLRSED | TSTAYMELSSLRSED |
| | LTISSLQPEDFA | WGQGTLVTVSSASTKGPS | TAVYYCARDAAGIRL | TAVYYCARDAAGIRL |
| | TYYCQSTYTAPW | VFPLAPSSKSTSGGTAAL | FADWGQGTLVTVSSA | FADWGQGTLVTVSSA |
| | TFGGGTKVEIKR | GCLVKDYFPEPVTVSWNS | STKGPSVFPLAPSSK | STKGPSVFPLAPSSK |
| | TVAAPSVFIFPP | GALTSGVHTFPAVLQSSG | STSGGTAALGCLVKD | STSGGTAALGCLVKD |
| | SDEQLKSGTASV | LYSLSSVVTVPSSSLGTQ | YFPEPVTVSWNSGAL | YFPEPVTVSWNSGAL |
| | VCLLNNFYPREA | TYICNVNHKPSNTKVDKK | TSGVHTFPAVLQSSG | TSGVHTFPAVLQSSG |
| | KVQWKVDNALQS | VEPKSCDKTHTCPPCPAP | LYSLSSVVTVPSSSL | LYSLSSVVTVPSSSL |
| | GNSQESVTEQDS | EAAGAPSVFLFPPKPKDT | GTQTYICNVNHKPSN | GTQTYICNVNHKPSN |
| | KDSTYSLSSTLT | LMISRTPEVTCVVVDVSH | TKVDKKVEPKSCDKT | TKVDKKVEPKSCDKT |
| | LSKADYEKHKVY | EDPEVKENWYVDGVEVHN | HTCPPCPAPEAAGAP | HTCPPCPAPEAAGAP |
| | ACEVTHQGLSSP | AKTKPREEQYNSTYRVVS | SVFLFPPKPKDTLMI | SVFLFPPKPKDTLMI |
| | VTKSENRGEC | VLTVLHQDWLNGKEYKCK | SRTPEVTCVVVDVSH | SRTPEVTCVVVDVSH |
| | (SEQ ID NO: | VSNKALPAPIEKTISKAK | EDPEVKENWYVDGVE | EDPEVKENWYVDGVE |
| | 557) | GQPREPQVYTLPPCREEM | VHNAKTKPREEQYNS | VHNAKTKPREEQYNS |
| | | TKNQVSLSCAVKGFYPSD | TYRVVSVLTVLHQDW | TYRVVSVLTVLHQDW |
| | | IAVEWESNGQPENNYKTT | LNGKEYKCKVSNKAL | LNGKEYKCKVSNKAL |
| | | PPVLDSDGSFFLVSKLTV | PAPIEKTISKAKGQP | PAPIEKTISKAKGQP |
| | | DKSRWQQGNVFSCSVMHE | REPQVCTLPPSREEM | REPQVCTLPPSREEM |
| | | ALHNHYTQKSLSLSPGSG | TKNQVSLWCLVKGFY | TKNQVSLWCLVKGFY |
| | | GGGSGGGGSGGGGSQGQD | PSDIAVEWESNGQPE | PSDIAVEWESNGQPE |
| | | RHMIRMRQLIDIVDQLKN | NNYKTTPPVLDSDGS | NNYKTTPPVLDSDGS |
| | | YVNDLVPEFLPAPEDVET | FFLYSKLTVDKSRWQ | FFLYSKLTVDKSRWQ |
| | | NCEWSAFSCFQKAQLKSA | QGNVFSCSVMHEALH | QGNVFSCSVMHEALH |
| | | NTGNNERIINVSIKKLKE | NHYTQKSLSLSPG | NHYTQKSLSLSPGK |
| | | KPPGGEGGGGGGGEGGCP | (SEQ ID NO: | (SEQ ID NO: |
| | | SCDSYEKKPPKEFLEREK | 559) | 560) |
| | | SLLQKMIHQHLSSRTHGS | | |
| | | EDS (SEQ ID NO: | | |
| | | 558) | | |
| xhCD8.1- | DIQMTQSPSSLS | QVQLVQSGAEVKKPGSSV | QVQLVQSGAEVKKPG | QVQLVQSGAEVKKPG |
| hIL21 | ASVGDRVTITCR | KVSCKASGGTESSYAISW | SSVKVSCKASGGTFS | SSVKVSCKASGGTES |
| v31.6 | ASQSIYGALNWY | VRQAPGQGLEWMGGIIPG | SYAISWVRQAPGQGL | SYAISWVRQAPGQGL |
| | QQKPGKAPKLLI | YATANYAQKFQGRVTITA | EWMGGIIPGYATANY | EWMGGIIPGYATANY |
| | YGASNLQSGVPS | DESTSTAYMELSSLRSED | AQKFQGRVTITADES | AQKFQGRVTITADES |
| | RFSGSGSGTDET | TAVYYCARDAAGIRLFAD | TSTAYMELSSLRSED | TSTAYMELSSLRSED |
| | LTISSLQPEDFA | WGQGTLVTVSSASTKGPS | TAVYYCARDAAGIRL | TAVYYCARDAAGIRL |
| | TYYCQSTYTAPW | VFPLAPSSKSTSGGTAAL | FADWGQGTLVTVSSA | FADWGQGTLVTVSSA |
| | TFGGGTKVEIKR | GCLVKDYFPEPVTVSWNS | STKGPSVFPLAPSSK | STKGPSVFPLAPSSK |
| | TVAAPSVFIFPP | GALTSGVHTFPAVLQSSG | STSGGTAALGCLVKD | STSGGTAALGCLVKD |
| | SDEQLKSGTASV | LYSLSSVVTVPSSSLGTQ | YFPEPVTVSWNSGAL | YFPEPVTVSWNSGAL |
| | VCLLNNFYPREA | TYICNVNHKPSNTKVDKK | TSGVHTFPAVLQSSG | TSGVHTFPAVLQSSG |
| | KVQWKVDNALQS | VEPKSCDKTHTCPPCPAP | LYSLSSVVTVPSSSL | LYSLSSVVTVPSSSL |
| | GNSQESVTEQDS | EAAGAPSVELFPPKPKDT | GTQTYICNVNHKPSN | GTQTYICNVNHKPSN |
| | KDSTYSLSSTLT | LMISRTPEVTCVVVDVSH | TKVDKKVEPKSCDKT | TKVDKKVEPKSCDKT |
| | LSKADYEKHKVY | EDPEVKENWYVDGVEVHN | HTCPPCPAPEAAGAP | HTCPPCPAPEAAGAP |
| | ACEVTHQGLSSP | AKTKPREEQYNSTYRVVS | SVFLFPPKPKDTLMI | SVFLFPPKPKDTLMI |
| | VTKSENRGEC | VLTVLHQDWLNGKEYKCK | SRTPEVTCVVVDVSH | SRTPEVTCVVVDVSH |
| | (SEQ ID NO: | VSNKALPAPIEKTISKAK | EDPEVKENWYVDGVE | EDPEVKENWYVDGVE |
| | 561) | GQPREPQVYTLPPCREEM | VHNAKTKPREEQYNS | VHNAKTKPREEQYNS |
| | | TKNQVSLSCAVKGFYPSD | TYRVVSVLTVLHQDW | TYRVVSVLTVLHQDW |
| | | IAVEWESNGQPENNYKTT | LNGKEYKCKVSNKAL | LNGKEYKCKVSNKAL |
| | | PPVLDSDGSFFLVSKLTV | PAPIEKTISKAKGQP | PAPIEKTISKAKGQP |
| | | DKSRWQQGNVFSCSVMHE | REPQVCTLPPSREEM | REPQVCTLPPSREEM |
| | | ALHNHYTQKSLSLSPGSG | TKNQVSLWCLVKGFY | TKNQVSLWCLVKGFY |
| | | GGGSGGGGSGGGGSQGQD | PSDIAVEWESNGQPE | PSDIAVEWESNGQPE |
| | | RHMIRMRQLIDIVDQLKN | NNYKTTPPVLDSDGS | NNYKTTPPVLDSDGS |
| | | YVNDLVPEFLPAPEDVET | FFLYSKLTVDKSRWQ | FFLYSKLTVDKSRWQ |
| | | NCEWSAFSCFQKAQLKSA | QGNVFSCSVMHEALH | QGNVFSCSVMHEALH |
| | | NTGNNERIINVSIKKLKF | NHYTQKSLSLSPG | NHYTQKSLSLSPGK |
| | | KPPGGEGGGGGGGEGGCP | (SEQ ID NO: | (SEQ ID NO: |
| | | SCDSYEKKPPKEFLEREK | 563) | 564) |
| | | SLLQKMIHQHLSSRTHGS | | |
| | | EDS (SEQ ID NO: | | |
| | | 562) | | |
| xhCD8.1- | DIQMTQSPSSLS | QVQLVQSGAEVKKPGSSV | QVQLVQSGAEVKKPG | QVQLVQSGAEVKKPG |
| hIL21 | ASVGDRVTITCR | KVSCKASGGTESSYAISW | SSVKVSCKASGGTFS | SSVKVSCKASGGTES |
| v31.23 | ASQSIYGALNWY | VRQAPGQGLEWMGGIIPG | SYAISWVRQAPGQGL | SYAISWVRQAPGQGL |
| | QQKPGKAPKLLI | YATANYAQKFQGRVTITA | EWMGGIIPGYATANY | EWMGGIIPGYATANY |
| | YGASNLQSGVPS | DESTSTAYMELSSLRSED | AQKFQGRVTITADES | AQKFQGRVTITADES |
| | RFSGSGSGTDFT | TAVYYCARDAAGIRLFAD | TSTAYMELSSLRSED | TSTAYMELSSLRSED |
| | LTISSLQPEDFA | WGQGTLVTVSSASTKGPS | TAVYYCARDAAGIRL | TAVYYCARDAAGIRL |
| | TYYCQSTYTAPW | VFPLAPSSKSTSGGTAAL | FADWGQGTLVTVSSA | FADWGQGTLVTVSSA |
| | TFGGGTKVEIKR | GCLVKDYFPEPVTVSWNS | STKGPSVFPLAPSSK | STKGPSVFPLAPSSK |
| | TVAAPSVFIFPP | GALTSGVHTFPAVLQSSG | STSGGTAALGCLVKD | STSGGTAALGCLVKD |
| | SDEQLKSGTASV | LYSLSSVVTVPSSSLGTQ | YFPEPVTVSWNSGAL | YFPEPVTVSWNSGAL |
| | VCLLNNFYPREA | TYICNVNHKPSNTKVDKK | TSGVHTFPAVLQSSG | TSGVHTFPAVLQSSG |
| | KVQWKVDNALQS | VEPKSCDKTHTCPPCPAP | LYSLSSVVTVPSSSL | LYSLSSVVTVPSSSL |
| | GNSQESVTEQDS | EAAGAPSVELFPPKPKDT | GTQTYICNVNHKPSN | GTQTYICNVNHKPSN |
| | KDSTYSLSSTLT | LMISRTPEVTCVVVDVSH | TKVDKKVEPKSCDKT | TKVDKKVEPKSCDKT |
| | LSKADYEKHKVY | EDPEVKENWYVDGVEVHN | HTCPPCPAPEAAGAP | HTCPPCPAPEAAGAP |
| | ACEVTHQGLSSP | AKTKPREEQYNSTYRVVS | SVFLFPPKPKDTLMI | SVFLFPPKPKDTLMI |
| | VTKSENRGEC | VLTVLHQDWLNGKEYKCK | SRTPEVTCVVVDVSH | SRTPEVTCVVVDVSH |
| | (SEQ ID NO: | VSNKALPAPIEKTISKAK | EDPEVKENWYVDGVE | EDPEVKFNWYVDGVE |
| | 565) | GQPREPQVYTLPPCREEM | VHNAKTKPREEQYNS | VHNAKTKPREEQYNS |
| | | TKNQVSLSCAVKGFYPSD | TYRVVSVLTVLHQDW | TYRVVSVLTVLHQDW |
| | | IAVEWESNGQPENNYKTT | LNGKEYKCKVSNKAL | LNGKEYKCKVSNKAL |
| | | PPVLDSDGSFFLVSKLTV | PAPIEKTISKAKGQP | PAPIEKTISKAKGQP |
| | | DKSRWQQGNVFSCSVMHE | REPQVCTLPPSREEM | REPQVCTLPPSREEM |
| | | ALHNHYTQKSLSLSPGSG | TKNQVSLWCLVKGFY | TKNQVSLWCLVKGFY |
| | | GGGSGGGGSGGGGSQGQD | PSDIAVEWESNGQPE | PSDIAVEWESNGQPE |
| | | RHMIRMRQLIDIVDQLKN | NNYKTTPPVLDSDGS | NNYKTTPPVLDSDGS |
| | | YVNDLVPEFLPAPEDVET | FFLYSKLTVDKSRWQ | FFLYSKLTVDKSRWQ |
| | | NCEWSAFSCFQKAQLKSA | QGNVFSCSVMHEALH | QGNVFSCSVMHEALH |
| | | NTGNNERIINVSIKKLKQ | NHYTQKSLSLSPG | NHYTQKSLSLSPGK |
| | | KPPGGEGGGGGGGEGGCP | (SEQ ID NO: | (SEQ ID NO: |
| | | SCDSYEKKPPKEFLEREK | 567) | 568 ) |
| | | SLLQKMIHQHLSSRTHGS | | |
| | | EDS (SEQ ID NO: | | |
| | | 566) | | |
| xhCD8.1- | DIQMTQSPSSLS | QVQLVQSGAEVKKPGSSV | QVQLVQSGAEVKKPG | QVQLVQSGAEVKKPG |
| hIL21 | ASVGDRVTITCR | KVSCKASGGTESSYAISW | SSVKVSCKASGGTFS | SSVKVSCKASGGTES |
| v31.48 | ASQSIYGALNWY | VRQAPGQGLEWMGGIIPG | SYAISWVRQAPGQGL | SYAISWVRQAPGQGL |
| | QQKPGKAPKLLI | YATANYAQKFQGRVTITA | EWMGGIIPGYATANY | EWMGGIIPGYATANY |
| | YGASNLQSGVPS | DESTSTAYMELSSLRSED | AQKFQGRVTITADES | AQKFQGRVTITADES |
| | RFSGSGSGTDET | TAVYYCARDAAGIRLFAD | TSTAYMELSSLRSED | TSTAYMELSSLRSED |
| | LTISSLQPEDFA | WGQGTLVTVSSASTKGPS | TAVYYCARDAAGIRL | TAVYYCARDAAGIRL |
| | TYYCQSTYTAPW | VFPLAPSSKSTSGGTAAL | FADWGQGTLVTVSSA | FADWGQGTLVTVSSA |
| | TFGGGTKVEIKR | GCLVKDYFPEPVTVSWNS | STKGPSVFPLAPSSK | STKGPSVFPLAPSSK |
| | TVAAPSVFIFPP | GALTSGVHTFPAVLQSSG | STSGGTAALGCLVKD | STSGGTAALGCLVKD |
| | SDEQLKSGTASV | LYSLSSVVTVPSSSLGTQ | YFPEPVTVSWNSGAL | YFPEPVTVSWNSGAL |
| | VCLLNNFYPREA | TYICNVNHKPSNTKVDKK | TSGVHTFPAVLQSSG | TSGVHTFPAVLQSSG |
| | KVQWKVDNALQS | VEPKSCDKTHTCPPCPAP | LYSLSSVVTVPSSSL | LYSLSSVVTVPSSSL |
| | GNSQESVTEQDS | EAAGAPSVELFPPKPKDT | GTQTYICNVNHKPSN | GTQTYICNVNHKPSN |
| | KDSTYSLSSTLT | LMISRTPEVTCVVVDVSH | TKVDKKVEPKSCDKT | TKVDKKVEPKSCDKT |
| | LSKADYEKHKVY | EDPEVKENWYVDGVEVHN | HTCPPCPAPEAAGAP | HTCPPCPAPEAAGAP |
| | ACEVTHQGLSSP | AKTKPREEQYNSTYRVVS | SVFLFPPKPKDTLMI | SVFLFPPKPKDTLMI |
| | VTKSENRGEC | VLTVLHQDWLNGKEYKCK | SRTPEVTCVVVDVSH | SRTPEVTCVVVDVSH |
| | (SEQ ID NO: | VSNKALPAPIEKTISKAK | EDPEVKENWYVDGVE | EDPEVKENWYVDGVE |
| | 569) | GQPREPQVYTLPPCREEM | VHNAKTKPREEQYNS | VHNAKTKPREEQYNS |
| | | TKNQVSLSCAVKGFYPSD | TYRVVSVLTVLHQDW | TYRVVSVLTVLHQDW |
| | | IAVEWESNGQPENNYKTT | LNGKEYKCKVSNKAL | LNGKEYKCKVSNKAL |
| | | PPVLDSDGSFFLVSKLTV | PAPIEKTISKAKGQP | PAPIEKTISKAKGQP |
| | | DKSRWQQGNVFSCSVMHE | REPQVCTLPPSREEM | REPQVCTLPPSREEM |
| | | ALHNHYTQKSLSLSPGSG | TKNQVSLWCLVKGFY | TKNQVSLWCLVKGFY |
| | | GGGSGGGGSGGGGSQGQD | PSDIAVEWESNGQPE | PSDIAVEWESNGQPE |
| | | RHMISMRQLIDIVDQLKN | NNYKTTPPVLDSDGS | NNYKTTPPVLDSDGS |
| | | YVNDLVPEFLPAPEDVET | FFLYSKLTVDKSRWQ | FFLYSKLTVDKSRWQ |
| | | NCEWSAFSCFQKAQLKSA | QGNVFSCSVMHEALH | QGNVFSCSVMHEALH |
| | | NTGNNERIINVSIKKLKR | NHYTQKSLSLSPG | NHYTQKSLSLSPGK |
| | | KPPGGEGGGGGGGEGGCP | (SEQ ID NO: | (SEQ ID NO: |
| | | SCDSYEKKPPKEFLEREK | 571) | 572) |
| | | SLLQKMIHQHLSSRTHGS | | |
| | | EDS (SEQ ID NO: | | |
| | | 570) | | |
| xhCD8.1- | DIQMTQSPSSLS | QVQLVQSGAEVKKPGSSV | QVQLVQSGAEVKKPG | QVQLVQSGAEVKKPG |
| hIL21 | ASVGDRVTITCR | KVSCKASGGTFSSYAISW | SSVKVSCKASGGTFS | SSVKVSCKASGGTES |
| v31.51 | ASQSIYGALNWY | VRQAPGQGLEWMGGIIPG | SYAISWVRQAPGQGL | SYAISWVRQAPGQGL |
| | QQKPGKAPKLLI | YATANYAQKFQGRVTITA | EWMGGIIPGYATANY | EWMGGIIPGYATANY |
| | YGASNLQSGVPS | DESTSTAYMELSSLRSED | AQKFQGRVTITADES | AQKFQGRVTITADES |
| | RFSGSGSGTDFT | TAVYYCARDAAGIRLFAD | TSTAYMELSSLRSED | TSTAYMELSSLRSED |
| | LTISSLQPEDFA | WGQGTLVTVSSASTKGPS | TAVYYCARDAAGIRL | TAVYYCARDAAGIRL |
| | TYYCQSTYTAPW | VFPLAPSSKSTSGGTAAL | FADWGQGTLVTVSSA | FADWGQGTLVTVSSA |
| | TFGGGTKVEIKR | GCLVKDYFPEPVTVSWNS | STKGPSVFPLAPSSK | STKGPSVFPLAPSSK |
| | TVAAPSVFIFPP | GALTSGVHTFPAVLQSSG | STSGGTAALGCLVKD | STSGGTAALGCLVKD |
| | SDEQLKSGTASV | LYSLSSVVTVPSSSLGTQ | YFPEPVTVSWNSGAL | YFPEPVTVSWNSGAL |
| | VCLLNNFYPREA | TYICNVNHKPSNTKVDKK | TSGVHTFPAVLQSSG | TSGVHTFPAVLQSSG |
| | KVQWKVDNALQS | VEPKSCDKTHTCPPCPAP | LYSLSSVVTVPSSSL | LYSLSSVVTVPSSSL |
| | GNSQESVTEQDS | EAAGAPSVFLFPPKPKDT | GTQTYICNVNHKPSN | GTQTYICNVNHKPSN |
| | KDSTYSLSSTLT | LMISRTPEVTCVVVDVSH | TKVDKKVEPKSCDKT | TKVDKKVEPKSCDKT |
| | LSKADYEKHKVY | EDPEVKENWYVDGVEVHN | HTCPPCPAPEAAGAP | HTCPPCPAPEAAGAP |
| | ACEVTHQGLSSP | AKTKPREEQYNSTYRVVS | SVFLFPPKPKDTLMI | SVFLFPPKPKDTLMI |
| | VTKSFNRGEC | VLTVLHQDWLNGKEYKCK | SRTPEVTCVVVDVSH | SRTPEVTCVVVDVSH |
| | (SEQ ID NO: | VSNKALPAPIEKTISKAK | EDPEVKENWYVDGVE | EDPEVKFNWYVDGVE |
| | 573) | GQPREPQVYTLPPCREEM | VHNAKTKPREEQYNS | VHNAKTKPREEQYNS |
| | | TKNQVSLSCAVKGFYPSD | TYRVVSVLTVLHQDW | TYRVVSVLTVLHQDW |
| | | IAVEWESNGQPENNYKTT | LNGKEYKCKVSNKAL | LNGKEYKCKVSNKAL |
| | | PPVLDSDGSFFLVSKLTV | PAPIEKTISKAKGQP | PAPIEKTISKAKGQP |
| | | DKSRWQQGNVFSCSVMHE | REPQVCTLPPSREEM | REPQVCTLPPSREEM |
| | | ALHNHYTQKSLSLSPGSG | TKNQVSLWCLVKGFY | TKNQVSLWCLVKGFY |
| | | GGGSGGGGSGGGGSQGQD | PSDIAVEWESNGQPE | PSDIAVEWESNGQPE |
| | | RHMIGMRQLIDIVDQLKN | NNYKTTPPVLDSDGS | NNYKTTPPVLDSDGS |
| | | YVNDLVPEFLPAPEDVET | FFLYSKLTVDKSRWQ | FFLYSKLTVDKSRWQ |
| | | NCEWSAFSCFQKAQLKSA | QGNVFSCSVMHEALH | QGNVFSCSVMHEALH |
| | | NTGNNERIINVSIKKLKR | NHYTQKSLSLSPG | NHYTQKSLSLSPGK |
| | | KPPGGEGGGGGGGEGGCP | (SEQ ID NO: | (SEQ ID NO: |
| | | SCDSYEKKPPKEFLEREK | 575) | 576) |
| | | SLLQKMIHQHLSSRTHGS | | |
| | | EDS (SEQ ID NO: | | |
| | | 574) | | |
| xhCD8v11- | EIVLTQSPGTLS | EVQLVESGGGLVQPGGSL | EVQLVESGGGLVQPG | EVQLVESGGGLVQPG |
| hIL21 | LSPGERATLSCR | RLSCAASGFTFSSYAMSW | GSLRLSCAASGFTFS | GSLRLSCAASGETES |
| v31.4 | ASQSVSSNLAWY | VRQAPGKGLEWVSDITYA | SYAMSWVRQAPGKGL | SYAMSWVRQAPGKGL |
| | QQKPGQAPRLLI | GGSTAYADSVKGRFTISR | EWVSDITYAGGSTAY | EWVSDITYAGGSTAY |
| | YGASSRATGIPD | DNAKNSLYLQMNSLRAED | ADSVKGRETISRDNA | ADSVKGRFTISRDNA |
| | RFSGSGSGTDFT | TAVYYCARSNAYAWDDAL | KNSLYLQMNSLRAED | KNSLYLQMNSLRAED |
| | LTISRLEPEDFA | DIWGQGTLVTVSSASTKG | TAVYYCARSNAYAWD | TAVYYCARSNAYAWD |
| | VYYCQQYGSSPP | PSVFPLAPSSKSTSGGTA | DALDIWGQGTLVTVS | DALDIWGQGTLVTVS |
| | VTFGQGTKVEIK | ALGCLVKDYFPEPVTVSW | SASTKGPSVFPLAPS | SASTKGPSVFPLAPS |
| | RTVAAPSVFIFP | NSGALTSGVHTEPAVLQS | SKSTSGGTAALGCLV | SKSTSGGTAALGCLV |
| | PSDEQLKSGTAS | SGLYSLSSVVTVPSSSLG | KDYFPEPVTVSWNSG | KDYFPEPVTVSWNSG |
| | VVCLLNNFYPRE | TQTYICNVNHKPSNTKVD | ALTSGVHTEPAVLQS | ALTSGVHTFPAVLQS |
| | AKVQWKVDNALQ | KKVEPKSCDKTHTCPPCP | SGLYSLSSVVTVPSS | SGLYSLSSVVTVPSS |
| | SGNSQESVTEQD | APEAAGAPSVFLFPPKPK | SLGTQTYICNVNHKP | SLGTQTYICNVNHKP |
| | SKDSTYSLSSTL | DTLMISRTPEVTCVVVDV | SNTKVDKKVEPKSCD | SNTKVDKKVEPKSCD |
| | TLSKADYEKHKV | SHEDPEVKENWYVDGVEV | KTHTCPPCPAPEAAG | KTHTCPPCPAPEAAG |
| | YACEVTHQGLSS | HNAKTKPREEQYNSTYRV | APSVFLFPPKPKDTL | APSVELFPPKPKDTL |
| | PVTKSENRGEC | VSVLTVLHQDWLNGKEYK | MISRTPEVTCVVVDV | MISRTPEVTCVVVDV |
| | (SEQ ID NO: | CKVSNKALPAPIEKTISK | SHEDPEVKENWYVDG | SHEDPEVKENWYVDG |
| | 577) | AKGQPREPQVYTLPPCRE | VEVHNAKTKPREEQY | VEVHNAKTKPREEQY |
| | | EMTKNQVSLSCAVKGFYP | NSTYRVVSVLTVLHQ | NSTYRVVSVLTVLHQ |
| | | SDIAVEWESNGQPENNYK | DWLNGKEYKCKVSNK | DWLNGKEYKCKVSNK |
| | | TTPPVLDSDGSFFLVSKL | ALPAPIEKTISKAKG | ALPAPIEKTISKAKG |
| | | TVDKSRWQQGNVFSCSVM | QPREPQVCTLPPSRE | QPREPQVCTLPPSRE |
| | | HEALHNHYTQKSLSLSPG | EMTKNQVSLWCLVKG | EMTKNQVSLWCLVKG |
| | | SGGGGSGGGGSGGGGSQG | FYPSDIAVEWESNGQ | FYPSDIAVEWESNGQ |
| | | QDRHMIRMRQLIDIVDQL | PENNYKTTPPVLDSD | PENNYKTTPPVLDSD |
| | | KNYVNDLVPEFLPAPEDV | GSFFLYSKLTVDKSR | GSFFLYSKLTVDKSR |
| | | ETNCEWSAFSCFQKAQLK | WQQGNVFSCSVMHEA | WQQGNVFSCSVMHEA |
| | | SANTGNNERIINVSIKKL | LHNHYTQKSLSLSPG | LHNHYTQKSLSLSPG |
| | | KEKPPGGEGGGGGGGEGG | (SEQ ID NO: | K (SEQ ID NO: |
| | | CPSCDSYEKKPPKEFLER | 579) | 580) |
| | | FKSLLQKMIHQHLSSRTH | | |
| | | GSEDS (SEQ ID NO: | | |
| | | 578) | | |
| xhCD8v11- | EIVLTQSPGTLS | EVQLVESGGGLVQPGGSL | EVQLVESGGGLVQPG | EVQLVESGGGLVQPG |
| hIL21 | LSPGERATLSCR | RLSCAASGFTFSSYAMSW | GSLRLSCAASGFTFS | GSLRLSCAASGFTES |
| v31.6 | ASQSVSSNLAWY | VRQAPGKGLEWVSDITYA | SYAMSWVRQAPGKGL | SYAMSWVRQAPGKGL |
| | QQKPGQAPRLLI | GGSTAYADSVKGRFTISR | EWVSDITYAGGSTAY | EWVSDITYAGGSTAY |
| | YGASSRATGIPD | DNAKNSLYLQMNSLRAED | ADSVKGRFTISRDNA | ADSVKGRFTISRDNA |
| | RFSGSGSGTDFT | TAVYYCARSNAYAWDDAL | KNSLYLQMNSLRAED | KNSLYLQMNSLRAED |
| | LTISRLEPEDFA | DIWGQGTLVTVSSASTKG | TAVYYCARSNAYAWD | TAVYYCARSNAYAWD |
| | VYYCQQYGSSPP | PSVFPLAPSSKSTSGGTA | DALDIWGQGTLVTVS | DALDIWGQGTLVTVS |
| | VTFGQGTKVEIK | ALGCLVKDYFPEPVTVSW | SASTKGPSVFPLAPS | SASTKGPSVFPLAPS |
| | RTVAAPSVFIFP | NSGALTSGVHTEPAVLQS | SKSTSGGTAALGCLV | SKSTSGGTAALGCLV |
| | PSDEQLKSGTAS | SGLYSLSSVVTVPSSSLG | KDYFPEPVTVSWNSG | KDYFPEPVTVSWNSG |
| | VVCLLNNFYPRE | TQTYICNVNHKPSNTKVD | ALTSGVHTFPAVLQS | ALTSGVHTEPAVLQS |
| | AKVQWKVDNALQ | KKVEPKSCDKTHTCPPCP | SGLYSLSSVVTVPSS | SGLYSLSSVVTVPSS |
| | SGNSQESVTEQD | APEAAGAPSVELFPPKPK | SLGTQTYICNVNHKP | SLGTQTYICNVNHKP |
| | SKDSTYSLSSTL | DTLMISRTPEVTCVVVDV | SNTKVDKKVEPKSCD | SNTKVDKKVEPKSCD |
| | TLSKADYEKHKV | SHEDPEVKFNWYVDGVEV | KTHTCPPCPAPEAAG | KTHTCPPCPAPEAAG |
| | YACEVTHQGLSS | HNAKTKPREEQYNSTYRV | APSVFLFPPKPKDTL | APSVELFPPKPKDTL |
| | PVTKSENRGEC | VSVLTVLHQDWLNGKEYK | MISRTPEVTCVVVDV | MISRTPEVTCVVVDV |
| | (SEQ ID NO: | CKVSNKALPAPIEKTISK | SHEDPEVKENWYVDG | SHEDPEVKENWYVDG |
| | 581) | AKGQPREPQVYTLPPCRE | VEVHNAKTKPREEQY | VEVHNAKTKPREEQY |
| | | EMTKNQVSLSCAVKGFYP | NSTYRVVSVLTVLHQ | NSTYRVVSVLTVLHQ |
| | | SDIAVEWESNGQPENNYK | DWLNGKEYKCKVSNK | DWLNGKEYKCKVSNK |
| | | TTPPVLDSDGSFFLVSKL | ALPAPIEKTISKAKG | ALPAPIEKTISKAKG |
| | | TVDKSRWQQGNVFSCSVM | QPREPQVCTLPPSRE | QPREPQVCTLPPSRE |
| | | HEALHNHYTQKSLSLSPG | EMTKNQVSLWCLVKG | EMTKNQVSLWCLVKG |
| | | SGGGGSGGGGSGGGGSQG | FYPSDIAVEWESNGQ | FYPSDIAVEWESNGQ |
| | | QDRHMIRMRQLIDIVDQL | PENNYKTTPPVLDSD | PENNYKTTPPVLDSD |
| | | KNYVNDLVPEFLPAPEDV | GSFFLYSKLTVDKSR | GSFFLYSKLTVDKSR |
| | | ETNCEWSAFSCFQKAQLK | WQQGNVFSCSVMHEA | WQQGNVFSCSVMHEA |
| | | SANTGNNERIINVSIKKL | LHNHYTQKSLSLSPG | LHNHYTQKSLSLSPG |
| | | KFKPPGGEGGGGGGGEGG | (SEQ ID NO: | K |
| | | CPSCDSYEKKPPKEFLER | 583) | (SEQ ID NO: |
| | | FKSLLQKMIHQHLSSRTH | | 584) |
| | | GSEDS | | |
| | | (SEQ ID NO: 582) | | |
| xhCD8v11- | EIVLTQSPGTLS | EVQLVESGGGLVQPGGSL | EVQLVESGGGLVQPG | EVQLVESGGGLVQPG |
| hIL21 | LSPGERATLSCR | RLSCAASGFTFSSYAMSW | GSLRLSCAASGFTFS | GSLRLSCAASGFTES |
| v31.23 | ASQSVSSNLAWY | VRQAPGKGLEWVSDITYA | SYAMSWVRQAPGKGL | SYAMSWVRQAPGKGL |
| | QQKPGQAPRLLI | GGSTAYADSVKGRFTISR | EWVSDITYAGGSTAY | EWVSDITYAGGSTAY |
| | YGASSRATGIPD | DNAKNSLYLQMNSLRAED | ADSVKGRFTISRDNA | ADSVKGRFTISRDNA |
| | RFSGSGSGTDFT | TAVYYCARSNAYAWDDAL | KNSLYLQMNSLRAED | KNSLYLQMNSLRAED |
| | LTISRLEPEDFA | DIWGQGTLVTVSSASTKG | TAVYYCARSNAYAWD | TAVYYCARSNAYAWD |
| | VYYCQQYGSSPP | PSVFPLAPSSKSTSGGTA | DALDIWGQGTLVTVS | DALDIWGQGTLVTVS |
| | VTFGQGTKVEIK | ALGCLVKDYFPEPVTVSW | SASTKGPSVFPLAPS | SASTKGPSVFPLAPS |
| | RTVAAPSVFIFP | NSGALTSGVHTEPAVLQS | SKSTSGGTAALGCLV | SKSTSGGTAALGCLV |
| | PSDEQLKSGTAS | SGLYSLSSVVTVPSSSLG | KDYFPEPVTVSWNSG | KDYFPEPVTVSWNSG |
| | VVCLLNNFYPRE | TQTYICNVNHKPSNTKVD | ALTSGVHTFPAVLQS | ALTSGVHTFPAVLQS |
| | AKVQWKVDNALQ | KKVEPKSCDKTHTCPPCP | SGLYSLSSVVTVPSS | SGLYSLSSVVTVPSS |
| | SGNSQESVTEQD | APEAAGAPSVELFPPKPK | SLGTQTYICNVNHKP | SLGTQTYICNVNHKP |
| | SKDSTYSLSSTL | DTLMISRTPEVTCVVVDV | SNTKVDKKVEPKSCD | SNTKVDKKVEPKSCD |
| | TLSKADYEKHKV | SHEDPEVKENWYVDGVEV | KTHTCPPCPAPEAAG | KTHTCPPCPAPEAAG |
| | YACEVTHQGLSS | HNAKTKPREEQYNSTYRV | APSVELFPPKPKDTL | APSVELFPPKPKDTL |
| | PVTKSENRGEC | VSVLTVLHQDWLNGKEYK | MISRTPEVTCVVVDV | MISRTPEVTCVVVDV |
| | (SEQ ID NO: | CKVSNKALPAPIEKTISK | SHEDPEVKENWYVDG | SHEDPEVKENWYVDG |
| | 585) | AKGQPREPQVYTLPPCRE | VEVHNAKTKPREEQY | VEVHNAKTKPREEQY |
| | | EMTKNQVSLSCAVKGFYP | NSTYRVVSVLTVLHQ | NSTYRVVSVLTVLHQ |
| | | SDIAVEWESNGQPENNYK | DWLNGKEYKCKVSNK | DWLNGKEYKCKVSNK |
| | | TTPPVLDSDGSFFLVSKL | ALPAPIEKTISKAKG | ALPAPIEKTISKAKG |
| | | TVDKSRWQQGNVFSCSVM | QPREPQVCTLPPSRE | QPREPQVCTLPPSRE |
| | | HEALHNHYTQKSLSLSPG | EMTKNQVSLWCLVKG | EMTKNQVSLWCLVKG |
| | | SGGGGSGGGGSGGGGSQG | FYPSDIAVEWESNGQ | FYPSDIAVEWESNGQ |
| | | QDRHMIRMRQLIDIVDQL | PENNYKTTPPVLDSD | PENNYKTTPPVLDSD |
| | | KNYVNDLVPEFLPAPEDV | GSFFLYSKLTVDKSR | GSFFLYSKLTVDKSR |
| | | ETNCEWSAFSCFQKAQLK | WQQGNVFSCSVMHEA | WQQGNVFSCSVMHEA |
| | | SANTGNNERIINVSIKKL | LHNHYTQKSLSLSPG | LHNHYTQKSLSLSPG |
| | | KQKPPGGEGGGGGGGEGG | (SEQ ID NO: | K (SEQ ID NO: |
| | | CPSCDSYEKKPPKEFLER | 587) | 588) |
| | | FKSLLQKMIHQHLSSRTH | | |
| | | GSEDS (SEQ ID NO: | | |
| | | 586) | | |
| xhCD8v11- | EIVLTQSPGTLS | EVQLVESGGGLVQPGGSL | EVQLVESGGGLVQPG | EVQLVESGGGLVQPG |
| hIL21 | LSPGERATLSCR | RLSCAASGFTESSYAMSW | GSLRLSCAASGFTES | GSLRLSCAASGFTES |
| v31.51 | ASQSVSSNLAWY | VRQAPGKGLEWVSDITYA | SYAMSWVRQAPGKGL | SYAMSWVRQAPGKGL |
| | QQKPGQAPRLLI | GGSTAYADSVKGRETISR | EWVSDITYAGGSTAY | EWVSDITYAGGSTAY |
| | YGASSRATGIPD | DNAKNSLYLQMNSLRAED | ADSVKGRFTISRDNA | ADSVKGRFTISRDNA |
| | RFSGSGSGTDFT | TAVYYCARSNAYAWDDAL | KNSLYLQMNSLRAED | KNSLYLQMNSLRAED |
| | LTISRLEPEDFA | DIWGQGTLVTVSSASTKG | TAVYYCARSNAYAWD | TAVYYCARSNAYAWD |
| | VYYCQQYGSSPP | PSVFPLAPSSKSTSGGTA | DALDIWGQGTLVTVS | DALDIWGQGTLVTVS |
| | VTFGQGTKVEIK | ALGCLVKDYFPEPVTVSW | SASTKGPSVFPLAPS | SASTKGPSVFPLAPS |
| | RTVAAPSVFIFP | NSGALTSGVHTFPAVLQS | SKSTSGGTAALGCLV | SKSTSGGTAALGCLV |
| | PSDEQLKSGTAS | SGLYSLSSVVTVPSSSLG | KDYFPEPVTVSWNSG | KDYFPEPVTVSWNSG |
| | VVCLLNNFYPRE | TQTYICNVNHKPSNTKVD | ALTSGVHTEPAVLQS | ALTSGVHTFPAVLQS |
| | AKVQWKVDNALQ | KKVEPKSCDKTHTCPPCP | SGLYSLSSVVTVPSS | SGLYSLSSVVTVPSS |
| | SGNSQESVTEQD | APEAAGAPSVELFPPKPK | SLGTQTYICNVNHKP | SLGTQTYICNVNHKP |
| | SKDSTYSLSSTL | DTLMISRTPEVTCVVVDV | SNTKVDKKVEPKSCD | SNTKVDKKVEPKSCD |
| | TLSKADYEKHKV | SHEDPEVKENWYVDGVEV | KTHTCPPCPAPEAAG | KTHTCPPCPAPEAAG |
| | YACEVTHQGLSS | HNAKTKPREEQYNSTYRV | APSVELFPPKPKDTL | APSVELFPPKPKDTL |
| | PVTKSENRGEC | VSVLTVLHQDWLNGKEYK | MISRTPEVTCVVVDV | MISRTPEVTCVVVDV |
| | (SEQ ID NO: | CKVSNKALPAPIEKTISK | SHEDPEVKENWYVDG | SHEDPEVKENWYVDG |
| | 589) | AKGQPREPQVYTLPPCRE | VEVHNAKTKPREEQY | VEVHNAKTKPREEQY |
| | | EMTKNQVSLSCAVKGFYP | NSTYRVVSVLTVLHQ | NSTYRVVSVLTVLHQ |
| | | SDIAVEWESNGQPENNYK | DWLNGKEYKCKVSNK | DWLNGKEYKCKVSNK |
| | | TTPPVLDSDGSFFLVSKL | ALPAPIEKTISKAKG | ALPAPIEKTISKAKG |
| | | TVDKSRWQQGNVESCSVM | QPREPQVCTLPPSRE | QPREPQVCTLPPSRE |
| | | HEALHNHYTQKSLSLSPG | EMTKNQVSLWCLVKG | EMTKNQVSLWCLVKG |
| | | SGGGGSGGGGSGGGGSQG | FYPSDIAVEWESNGQ | FYPSDIAVEWESNGQ |
| | | QDRHMIGMRQLIDIVDQL | PENNYKTTPPVLDSD | PENNYKTTPPVLDSD |
| | | KNYVNDLVPEFLPAPEDV | GSFFLYSKLTVDKSR | GSFFLYSKLTVDKSR |
| | | ETNCEWSAFSCFQKAQLK | WQQGNVFSCSVMHEA | WQQGNVFSCSVMHEA |
| | | SANTGNNERIINVSIKKL | LHNHYTQKSLSLSPG | LHNHYTQKSLSLSPG |
| | | KRKPPGGEGGGGGGGEGG | (SEQ ID NO: | K (SEQ ID NO: |
| | | CPSCDSYEKKPPKEFLER | 591) | 592) |
| | | FKSLLQKMIHQHLSSRTH | | |
| | | GSEDS (SEQ ID NO: | | |
| | | 590) | | |

### Methods of Making Polypeptides

Further provided herein are polynucleotides (e.g., isolated polynucleotides) encoding any of the antibodies, antibody fragments, and fusion proteins described herein. Further provided herein are vectors (e.g., expression vectors) encoding any of the antibodies, antibody fragments, and fusion proteins described herein.

Further provided herein are host cells (e.g., isolated host cells or host cell lines) comprising any of the polynucleotides or vectors described herein.

Further provided herein are methods of producing any of the antibodies, antibody fragments, and fusion proteins described herein. In some embodiments, the methods comprise culturing a host cell of the present disclosure under conditions suitable for production of the antibody, antibody fragment, or fusion protein. In some embodiments, the methods further comprise recovering the antibody, antibody fragment, or fusion protein.

Antibodies, antibody fragments, and fusion proteins may be produced using recombinant methods, e.g., as exemplified infra. In some embodiments, nucleic acid encoding the antibody/fusion protein can be isolated and inserted into a replicable vector for further cloning or for expression. DNA encoding the antibody/fusion protein may be readily isolated and sequenced using conventional procedures (e.g., via oligonucleotide probes capable of binding specifically to genes encoding the heavy and light chains of the antibody/fragment). Many vectors are known in the art; vector components generally include, but are not limited to, one or more of the following: a signal sequence, an origin of replication, one or more marker genes, an enhancer element, a promoter, and a transcription termination sequence. Suitable host cells for cloning or expressing the DNA in the vectors herein are the prokaryote, yeast, or higher eukaryote cells. When using recombinant techniques, the antibody/fusion protein can be produced intracellularly, in the periplasmic space, or directly secreted into the medium. If the antibody/fragment is produced intracellularly, the particulate debris, either host cells or lysed fragments, are removed, for example, by centrifugation or ultrafiltration. Where the antibody/fusion protein is secreted into the medium, supernatants from such expression systems are generally first concentrated using a commercially available protein concentration filter.

### Pharmaceutical Compositions

In some embodiments, a fusion protein of the present disclosure is part of a pharmaceutical composition, e.g., including the fusion protein and one or more pharmaceutically acceptable carriers. Pharmaceutical compositions and formulations as described herein can be prepared by mixing the active ingredients (such as a fusion protein) having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives; low molecular weight (less than about 10 residues) polypeptides; proteins, such as serum albumin, gelatin, or immunoglobulins; hydrophilic polymers such as polyvinylpyrrolidone; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). In some embodiments, a fusion protein of the present disclosure is lyophilized.

In some embodiments, the IL-21 fusion protein and the IL-2 fusion protein are formulated in the same pharmaceutical composition. In some embodiments, the IL-21 fusion protein and the IL-2 fusion protein are formulated in separate pharmaceutical compositions. In some embodiments, the IL-21 fusion protein and the IL-2 fusion protein are administered by the same route of administration. In some embodiments, the IL-21 fusion protein and the IL-2 fusion protein are administered by different routes of administration.

In some embodiments, the route of administration of the pharmaceutical composition is in accord with known methods, e.g. orally, through injection by intravenous, intraperitoneal, intracerebral (intra-parenchymal), intracerebroventricular, intramuscular, subcutaneously, intra-ocular, intraarterial, intraportal, or intralesional routes; by sustained release systems or by implantation devices. In some embodiments, the compositions can be administered by bolus injection or continuously by infusion, or by implantation device. In some embodiments, individual elements of the combination therapy may be administered by different routes.

In some embodiments, the composition can be administered locally via implantation of a membrane, sponge or another appropriate material onto which the desired molecule has been absorbed or encapsulated. In some embodiments, where an implantation device is used, the device can be implanted into any suitable tissue or organ, and delivery of the desired molecule can be via diffusion, timed-release bolus, or continuous administration. In certain embodiments, it can be desirable to use a pharmaceutical composition comprising an IL-21 fusion protein and/or an IL-2 fusion protein in an ex vivo manner. In such instances, cells, tissues and/or organs that have been removed from the patient are exposed to a pharmaceutical composition comprising an IL-21 fusion protein and/or an IL-2 fusion protein, after which the cells, tissues and/or organs are subsequently implanted back into the patient.

In some embodiments, an 1L-21 fusion protein and/or an IL-2 fusion protein can be delivered by implanting certain cells that have been genetically engineered, using methods such as those described herein, to express and secrete the polypeptides. In some embodiments, such cells can be animal or human cells, and can be autologous, heterologous, or xenogeneic. In certain embodiments, the cells can be immortalized. In some embodiments, in order to decrease the chance of an immunological response, the cells can be encapsulated to avoid infiltration of surrounding tissues. In some embodiments, the encapsulation materials are typically biocompatible, semi-permeable polymeric enclosures or membranes that allow the release of the protein product(s) but prevent the destruction of the cells by the patient's immune system or by other detrimental factors from the surrounding tissues.

### Combination Methods

In some embodiments, the disclosure provides a method for using IL-2 fusion proteins and IL-21 fusions proteins, as described herein, in combination. In some embodiments, an IL-2 fusion protein and an IL-21 fusion protein are administered concurrently, sequentially, or simultaneously. In some embodiments, an IL-2 fusion protein and an IL-21 fusion protein are administered concurrently. In some embodiments, an IL-2 fusion protein and an IL-21 fusion protein are administered sequentially. In some embodiments, an IL-2 fusion protein and an IL-21 fusion protein are administered simultaneously.

In some embodiments, the method comprises administering an IL-2 fusion protein described herein and an IL-21 fusion protein described herein, to a subject in need thereof. In some embodiments, the method comprises administering an IL-2 fusion protein described herein to a subject in need thereof that has received or is receiving treatment with an IL-21 fusion protein described herein. In some embodiments, the method comprises administering an IL-21 fusion protein described herein to a subject in need thereof that has received or is receiving treatment with an IL-2 fusion protein described herein.

In some embodiments, the method comprises administering an additional therapeutic. In some embodiments, the additional therapeutic is a cell therapy (e.g., an adoptive cell therapy, a T-cell therapy, such as a CAR-T cell therapy, a TCR-T cell therapy, a tumor infiltrating lymphocyte (TIL) therapy), a cancer vaccine, an anti-angiogenesis agent, an antibody drug conjugate, a chemotherapeutic agent, an immune stimulating agent, an immune priming agent or immune checkpoint inhibitor (ICI). In some embodiments, the chemotherapeutic agent is a kinase inhibitor, antimetabolite, cytotoxin or cytostatic agent, anti-hormonal agent, platinum-based chemotherapeutic agent, methyltransferase inhibitor, antibody, or anti-cancer peptide.

In some embodiments, the immune checkpoint inhibitor targets PD-L1, PD-1, CTLA-4, CEACAM, LAIR1, CD160, 2B4, CD80, CD86, CD276, VTCN1, HVEM, KIR, A2AR, MHC class I, MHC class II, GALS, adenosine, TGFR, OX40, CD137, CD40, CD47, TREM1, TREM2, HLA-G, CCR4, CCR8, CD39, CD73, IDO, CSF1R, TIM-3, BTLA, VISTA, LAG-3, TIGIT, IDO, MICA/B, LILRB4, SIGLEC-15, or arginase, including without limitation an inhibitor of PD-1 (e.g., an anti-PD-1 antibody), PD-L1 (e.g., an anti-PD-L1 antibody), or CTLA-4 (e.g., an anti-CTLA-4 antibody).

Examples of anti-PD-1 antibodies include, without limitation, pembrolizumab, nivolumab, cemiplimab, zimberelimab (Arcus), sasanlimab (Pfizer), JTX-4014, spartalizumab (PDR001; Novartis), camrelizumab (SHR1210; Jiangsu HengRui Medicine), sintilimab (1B1308; Innovent and Eli Lilly), tislelizumab (BGB-A317), toripalimab (JS 001), dostarlimab (TSR-042, WBP-285), INCMGA00012 (MGA012), AMP-224, and AMP-514 (MEDI0680). Examples of anti-PD-L1 antibodies include, without limitation, atezolizumab, avelumab, durvalumab, KN035, and CK-301 (Checkpoint Therapeutics). Examples of PD-L1 inhibitors (non-antibody based) include, without limitation, AUNP12, CA-170, and BMS-986189. Examples of anti-CTLA-4 antibodies include, without limitation, ipilimumab, tremelimumab, BMS-986218, BMS-986249, BMS-986288, HBM4003, ONC-392, KN044, ADG116, ADU-1604, AGEN1181, AGEN1884, MK-1308, and REGN4659.

In some embodiments, the methods described herein treat a disease in a subject. In some embodiments, the disease is a cancer. In some embodiments, the disease is a chronic infection. In some embodiments the disease is selected for the group consisting of an autoimmune disease, inflammation, autoimmune disease, atopic diseases, paraneoplastic autoimmune diseases, cartilage inflammation, arthritis, rheumatoid arthritis, juvenile arthritis, juvenile rheumatoid arthritis, pauciarticular juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reiter's Syndrome, SEA Syndrome (Seronegativity, Enthesopathy, Arthropathy Syndrome), juvenile dermatomyositis, juvenile psoriatic arthritis, juvenile scleroderma, juvenile systemic lupus erythematosus, juvenile vasculitis, pauciarticular rheumatoid arthritis, polyarticular rheumatoid arthritis, systemic onset rheumatoid arthritis, ankylosing spondylitis, enteropathic arthritis, reactive arthritis, Reiter's Syndrome, SEA Syndrome (Seronegativity, Enthesopathy, Arthropathy Syndrome), dermatomyositis, psoriatic arthritis, scleroderma, vasculitis, myelitis, polymyositis, dermatomyositis, polyarteritis nodosa, Wegener's granulomatosis, arteritis, esophagitis rheumatica, sarcoidosis, sclerosis, primary biliary sclerosis, sclerosing cholangitis, Sjogren's syndrome, psoriasis, plaque psoriasis, guttate psoriasis, inverse psoriasis, pustular psoriasis, erythrodermic psoriasis, dermatitis, atopic dermatitis, atherosclerosis, lupus, Still's disease, Systemic Lupus Erythematosus (SLE), myasthenia gravis, inflammatory bowel disease (IBD), Crohn's disease, ulcerative colitis, celiac disease, multiple sclerosis (MS), asthma, COPD, rhinosinusitis, rhinosinusitis with polyps, eosinophilic esophagitis, eosinophilic bronchitis, Guillain-Barre disease, Type I diabetes mellitus, thyroiditis (e.g., Graves' disease), Addison's disease, Raynaud's phenomenon, autoimmune hepatitis, GVHD, transplantation rejection, kidney damage, hepatitis C-induced vasculitis, or spontaneous loss of pregnancy.

In some embodiments, the disease is a cancer. In some embodiments, the cancer is acute lymphoblastic leukemia (ALL) (including non T cell ALL), acute myeloid leukemia, B cell prolymphocytic leukemia, B cell acute lymphoid leukemia (“BALL”), blastic plasmacytoid dendritic cell neoplasm, Burkitt's lymphoma, chronic lymphocytic leukemia (CLL), chronic my elogenous leukemia (CML), chronic myeloid leukemia, chronic or acute leukemia, diffuse large B cell lymphoma (DLBCL), follicular lymphoma (FL), hairy cell leukemia, Hodgkin's Disease, malignant lymphoproliferative conditions, MALT lymphoma, mantle cell lymphoma, Marginal zone lymphoma, monoclonal gammopathy of undetermined significance (MGUS), multiple nyeloma, myelodysplasia and myelodysplastic syndrome, non-Hodgkin's lymphoma (NHL), plasma cell proliferative disorder (including asymptomatic myeloma, smoldering multiple myeloma or indolent myeloma), plasmablastic lymphoma, plasmacytoid dendritic cell neoplasm, plasmacytomas (including plasma cell dyscrasia; solitary myeloma; solitary plasmacytoma; extramedullary plasmacytoma; and multiple plasmacytoma), POEMS syndrome (also known as Crow-Fukase syndrome; Takatsuki disease; and PEP syndrome), primary mediastinal large B cell lymphoma (PMBC), small cell- or a large cell-follicular lymphoma, splenic marginal zone lymphoma (SMZL), systemic amyloid light chain amyloidosis, T cell acute lymphoid leukemia (“TALL”), T cell lymphora, transformed follicular lymphoma, or Waldenstromn macroglobulinemia, Mantle cell lymphoma (MCL), Transformed follicular lymphoma (TFL), Primary mediastinal B cell lymphoma (PMBCL), Multiple myeloma, Hairy cell lymphoma/leukemia, lung cancer, small-cell hung cancer, non-small cell lung (NSCL) cancer, bronchioloalveolar cell lung cancer, squamous cell cancer, adenocarcinoma of the lung, squamous carcinoma of the lung, cancer of the peritoneum, head and neck cancer, bone cancer, pancreatic cancer, skin cancer, cancer of the head or neck, cutaneous or intraocular melanoma, thyroid cancer, uterine cancer, gastrointestinal cancer, ovarian cancer, rectal cancer, cancer of the anal region, stomach cancer, gastric cancer, colon cancer, breast cancer, endometrial carcinoma, uterine cancer, carcinoma of the fallopian tubes, carcinoma of the cervix, carcinoma of the vagina, vulval cancer, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, bladder cancer, liver cancer, hepatoma, hepatocellular cancer, cervical cancer, salivary gland carcinoma, biliary cancer, neoplasms of the central nervous system (CNS), spinal axis tumors, brain stein glioma, glioblastoma multiforme, astrocytoma, schwannomas, ependymomas, medulloblastomas, meningiomas, squamous cell carcinomas, pituitary adenoma and Ewing's sarcoma, including refractory versions of any of the above cancers, or a combination of one or more of the above cancers.

In some embodiments, the disclosure provides a method for reducing or inhibiting tumor growth in a subject, comprising administering to the subject an IL-21 fusion protein and an IL-2 fusion protein described herein. In some embodiments the IL-21 fusion protein is administered prior to the IL-2 fusion protein. In some embodiments, the IL-2 fusion protein is administered prior to the IL-21 fusion protein. In some embodiments, the IL-2 fusion protein and the IL-21 fusion protein are administered sequentially, concurrently, or simultaneously.

### Kits

In some embodiments, the disclosure provides a kit comprising an IL-21 fusion protein and an IL-2 fusion protein, as disclosed herein, and instructions for use. The kits may comprise, in a suitable container, an IL-2 fusion protein, an IL-21 fusion protein, one or more controls, and various buffers, reagents, enzymes and other standard ingredients well known in the art. Certain embodiments include a kit with an TL-2 fusion protein and an IL-21 fusion protein in the same vial. In certain embodiments, a kit includes an IL-2 fusion protein and an IL-21 fusion protein in separate vials.

In some embodiments, the disclosure provides a kit comprising an IL-21 fusion protein as disclosed herein, and instructions for administering the IL-21 fusion protein to a patient in need thereof that has received or is receiving treatment with an IL-2 fusion protein. In some embodiments, the disclosure provides a kit comprising an IL-2 fusion protein as disclosed herein, and instructions for administering the IL-2 fusion protein to a patient in need thereof that has received or is receiving treatment with an IL-21 fusion protein.

The container can include at least one vial, well, test tube, flask, bottle, syringe, or other container means, into which an IL-21 fusion protein and/or an IL-2 fusion protein, may be placed, and in some instances, suitably aliquoted. Where an additional component is provided, the kit can contain additional containers into which this component may be placed. The kits can also include a means for containing an I L-2 fusion protein and/or an IL-21 fusion protein, and any other reagent containers in close confinement for commercial sale. Such containers may include injection or blow-molded plastic containers into which the desired vials are retained. Containers and/or kits can include labeling with instructions for use and/or warnings.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1: Construction of Mouse Surrogates of Cis-Targeted IL-21 and IL-2

### Materials and Methods

### Cloning of Murine Fusion Constructs

Murine IL-21 or IL-2 portions of the constructs were cloned in frame with the heavy chain using a (G₄S)₃ 15-mer linker between the C-terminus of the mouse IgG2a heavy chain and the N-terminus of IL-21. The C-terminal lysine residue of the IgG heavy chain was eliminated after fusing the IL-21 portion. To generate the constructs with asymmetric geometry, electrostatic modifications were introduced into the CH3 domains of the Fc region to facilitate heterodimerization. Specifically, the positively charged modifications carried the E356K and D399K mutations in the CH3 domain, whereas the negatively charged modifications carried the K409E and K439D mutations in the CH3 domain (EU numbering). To abolish FcγR binding/effector function and prevent FcR co-activation, mutations L234A/L235A/P329G (EU numbering) were introduced into the CH2 domain of each of the IgG heavy chains or the Fc region. The expression of the antibody-IL-21 fusion constructs was driven by an CMV promoter and transcription terminated by a synthetic polyA signal sequence located downstream of the coding sequence.

Purification of Fusion Proteins with IL-21 and IL-2 Polypeptides

Constructs encoding fusion proteins with IL-21 polypeptides as used in the examples were produced by co-transfecting exponentially growing Expi293 cells with the mammalian expression vectors using polyethylenimine (PEI). Supernatants were collected after 5 days of culture. IL-21 fusion constructs were first purified by affinity chromatography using a protein A matrix. The protein A column was equilibrated and washed in phosphate-buffered saline (PBS). The fusion constructs were eluted with 100 mM sodium citrate, pH 3.6 directly into 1 M Tris-HCl, pH 9 neutralization buffer. The eluted fractions were pooled and diluted into 20 mM MES, 25 mM sodium chloride pH 5.5 for further purification by ion-exchange chromatography (Mono-S, Cytiva) to separate heterodimers from homodimers. After loading the protein, the column was washed with 20 mM MES, pH 5.5. The protein was then eluted with a gradient of sodium chloride from 25 mM NaCl to either 500 mM NaCl for IL-21 fusion proteins or 300 mM NaCl for IL-2 fusion proteins in 20 mM MES, pH 5.5 buffer. The major eluent peak corresponding to the heterodimer was collected and analyzed by analytical size exclusion chromatography (Superdex 200 Increase 5/150 GL, Cytiva). For in vitro studies, the protein was dialyzed into PBS if the % monomer was ≥98%. Otherwise, and for all in vivo studies, the ion exchange eluent peak was polished by size exclusion chromatography (HiLoad Superdex 200 pg 16/600 or 26/600, Cytiva) in PBS.

The protein concentration of purified fusion constructs was determined by measuring the optical density (OD) at 280 nm, using the molar extinction coefficient calculated on the basis of the amino acid sequence. Purity, integrity, and monomeric state of the fusion constructs were analyzed by SDS-PAGE in the presence and absence of a reducing agent (5 mM 1,4-dithiothreitol) and stained with Coomassie blue (SimpleBlue™ SafeStain, Invitrogen). The NuPAGE® Pre-Cast gel system (Invitrogen) was used according to the manufacturer's instructions (4-20% Tris-glycine gels or 3-12% Bis-Tris). The aggregate content of immunoconjugate samples was analyzed using a Superdex 200 5/150 GL analytical size-exclusion column (Cytiva).

### MC38 Mouse Tumor Model

C57BL6 female mice (Jackson Labs) at 8-10 weeks of age were housed and acclimated at the vivarium facility. Cultured MC38 cells were harvested and resuspended in serum free media (1× DMEM, Sigma D6429) at 1.5×10⁷ cells/mL for implantation. Mice were shaved and 1.5×10⁶ cells (100 μL) are implanted subcutaneously above rear hind leg. Tumors were measured until tumor volume of 60-120 mm3 was reached at approximately 8 days post implantation (TV=width*width*length*0.5). Mice were then randomized into groups by tumor volume. Following randomization, each mouse was weighed and dosed subcutaneously with therapeutic agents under the scruff of the neck. Tumor volumes and body weights were measured every 3-4 days until either end of study (30-40 days post initial dose) or max tumor volume (2000 mm³) was reached. At end of study mice were euthanized by CO₂ with appropriate secondary euthanasia.

### Results

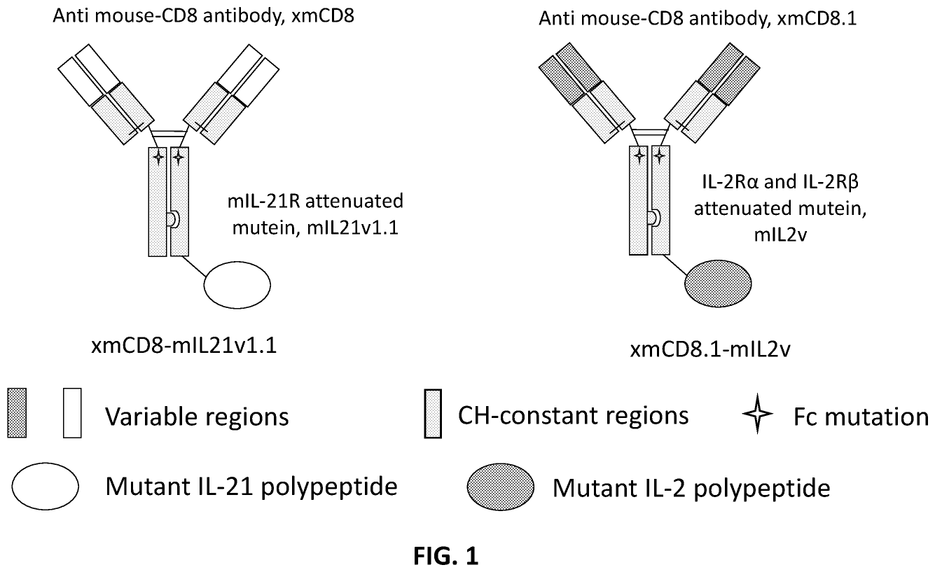

Murine CD8-cis targeted IL-21 and IL-2 fusion protein surrogates were designed as previously described (as described in international patent application nos. PCT/US20/36454, filed Jun. 5, 2020, PCT/US21/56312, filed Oct. 22, 2021, and PCT/US2022/026584, filed Apr. 27, 2022, each of which is hereby incorporated by reference in its entirety). FIG. 1 shows a schematic of these fusion proteins. xmCD8-mIL21v1.1 contains an anti-mouse CD8 antibody fused to an affinity-attenuated mouse IL-21 variant, mIL21v1.1. xmCD8.1-mIL2v contains an alternate anti-mouse CD8 antibody fused to an affinity-attenuated mouse IL-2, mIL2v. Table 11 shows the sequence of the mouse IL-2 and IL-21 muteins. The xmCD8 and xmCD8.1 antibodies target the same epitope (targeting CD8ab and CD8b) but have different binding affinities. Each antibody was derived from a parental antibody described in PCT/US20/36454 as binding CD8ab or CD8b, which has been reported as not blocking CD8+ T cell activation.

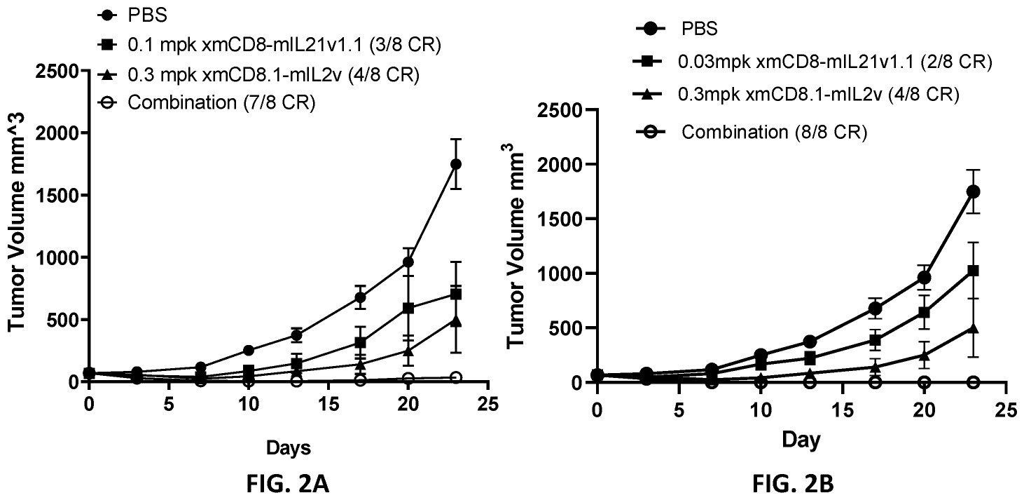

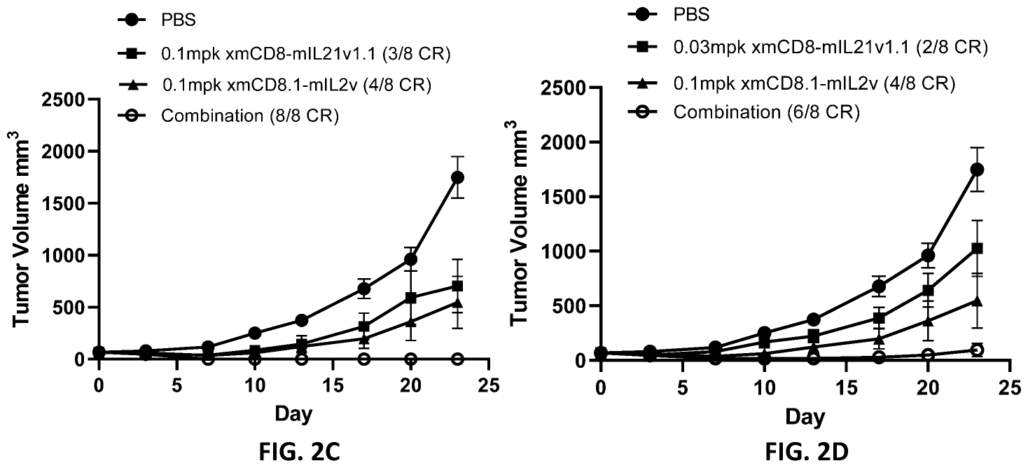

FIGS. 2A-2D show efficacy data in the MC38 model for either single surrogate or combinations of two surrogates dosed simultaneously at different dose levels. Single agent versus combinations are shown for 0.3 mg/kg xmCD8.1-mIL2v and 0.1 mg/kg xmCD8-mIL21v1.1 (FIG. 2A), 0.3 mg/kg xmCD8.1-mIL2v and 0.03 mg/kg xmCD8-mIL21v1.1 (FIG. 2B), 0.1 mg/kg xmCD8.1-mIL2v and 0.1 mg/kg xmCD8-mIL21v1.1 (FIG. 2C), 0.1 mg/kg xmCD8.1-mIL2v and 0.03 mg/kg xmCD8-mIL21v1.1 (FIG. 2D). In all cases, the combination shows greater efficacy based on mean tumor volume and the number of complete responses (CRs) compared to each of the single agent treatments.

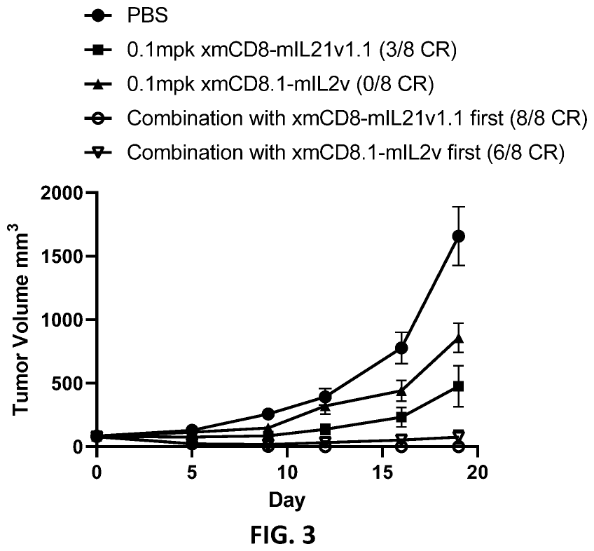

FIG. 3 shows efficacy data in the MC38 model for either single surrogate or combinations of two surrogates dosed in a sequential fashion. Separate groups were tested with either xmCD8-mIL21v1.1 dosed first, or xmCD8.1-mIL2v dosed first. In all cases, dosing was staggered by 1 hour. Single agent versus combination treatments is shown for 0.1 mg/kg xmCD8.1-mIL2v and 0.1 mg/kg xmCD8-mIL21v1.1 FIG. 3 shows that combinations regardless of order show synergistic effects, the combination shows greater efficacy based on mean tumor volume and the number of complete responses (CRs) compared to each of the single agent treatments.

TABLE 11
Engineered mouse IL-21 and IL-2 muteins

| Mouse | | |
|---|---|---|
| cytokine | | SEQ |
| mutein | Amino Acid Sequence | ID NO |
| mIL-21 | QGPDRLLIRLRHLIDIVEQLKIYENDLDPELLSAPQDVKGHCE | 593 |
| v1.1 | HAAFACFQKAKLKPSNPGNNKTFIIDLVAQLEERLPAGEGGEG | |
| | QEHIAKCPSCDSYEKRTPKEFLERLKWLLQKMIHQHLS | |
| mIL-2v | APTSSSTSSSTAEAQQQQQQQHLEQLLMDLQELLSRME | 594 |
| | NYRNLKLPEMLTAKFALPKQATELKDLQCLEDELGPLRHVLDL | |
| | TQSKSFQLEDAENFISSIRVTVVKLKGSDNTFECQFDDESATV | |
| | VDELRRWIAFAQSIISTSPQ | |

Embodiment 1. A method of treating a subject having a disease, the method comprising administering to the subject:
(a) an effective amount of an IL-21 fusion protein comprising (i) a first antibody or antigen-binding fragment thereof that specifically binds to human CD8, and (ii) an IL-21 polypeptide; and
(b) an effective amount of an IL-2 fusion protein comprising (i) an second antibody or antigen-binding fragment thereof that specifically binds human CD8, and (ii) an IL-2 polypeptide.

Embodiment 2. A method of treating a subject having a disease, wherein the subject has received or is receiving treatment with an IL-21 fusion protein, the method comprising: administering to the subject an effective amount of an IL-2 fusion protein, wherein the IL-21 fusion protein comprises (i) a first antibody or antigen-binding fragment thereof that specifically binds human CD8, and (ii) an IL-21 polypeptide, and wherein the IL-2 fusion protein comprises (i) a second antibody or antigen-binding fragment thereof that specifically binds human CD8, and (ii) an IL-2 polypeptide.

Embodiment 3. A method of treating a subject having a disease, wherein the subject has received or is receiving treatment with an IL-2 fusion protein, the method comprising: administering to the subject an effective amount of an IL-21 fusion protein, wherein the IL-21 fusion protein comprises (i) a first antibody or antigen-binding fragment thereof that specifically binds human CD8, and (ii) an IL-21 polypeptide, and wherein the IL-2 fusion protein comprises (i) a second antibody or an antigen-binding fragment thereof that specifically binds human CD8, (ii) an IL-2 polypeptide.

Embodiment 4. The method of any one of Embodiments 1-3, wherein the disease comprises a cancer.

Embodiment 5. The method of any one of Embodiments 1-3, wherein the disease comprises an infectious disease.

Embodiment 6. The method of any of the preceding Embodiments, wherein the treating induces an immune response in the subject.

Embodiment 7. The method of Embodiment 6, wherein the treating induces a T cell response in the subject.

Embodiment 8. The method of any of Embodiments 1-4 and 6-7, wherein the treating delays cancer progression in the subject.

Embodiment 9. The method of any of Embodiments 1-4 and 6-8, wherein the treating reduces tumor volume in the subject.

Embodiment 10. The method of any of Embodiments 1-4 and 6-9, wherein the treating reduces or inhibits tumor growth in the subject.

Embodiment 11. The method of any of Embodiments 1-4 and 6-10, wherein the treating delays cancer progression, reduces tumor volume, and/or reduces or inhibits tumor growth in the subject more than treatment with the IL-2 fusion protein alone in a subject.

Embodiment 12. The method of any of Embodiments 1-4 and 6-10, wherein the treating delays cancer progression, reduces tumor volume, and/or reduces or inhibits tumor growth in the subject more than treatment with the IL-21 fusion protein alone in a subject.

Embodiment 13. The method of any one of Embodiments 1-12, wherein the first antibody or antigen-binding fragment thereof and the second antibody or antigen-binding fragment thereof are the same.

Embodiment 14. The method of any one of Embodiments 1-12, wherein the first antibody or antigen-binding fragment thereof and the second antibody or antigen-binding fragment thereof are different.

Embodiment 15. The method of any one of Embodiments 1-14, wherein the first antibody or antigen-binding fragment thereof and the second antibody or antigen-binding fragment thereof both bind to a cell expressing a human CD8ab heterodimer on its surface with an EC50 that is less than 1000 nM.

Embodiment 16. The method of any one of Embodiments 1-15, wherein the first antibody or antigen-binding fragment thereof and the second antibody or antigen-binding fragment thereof both bind human CD8+ T cells.

Embodiment 17. The method of any one of Embodiments 1-16, wherein the first antibody or antigen-binding fragment thereof comprises a heavy chain and a light chain, wherein the heavy chain comprises a variable (VH) domain; and the light chain comprises a light chain variable (VL) domain, and wherein in the first antibody or antigen-binding fragment thereof:
the VH domain comprises a CDR-H1 comprising the amino acid sequence of SEQ ID NO: 13, a CDR-H2 comprising the amino acid sequence of SEQ ID NO: 14, and a CDR-H3 comprising the amino acid sequence of SEQ ID NO: 15; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 18;
the VH domain comprises a CDR-H1 comprising the amino acid sequence of SEQ ID NO: 19, a CDR-H2 comprising the amino acid sequence of SEQ ID NO: 20, and a CDR-H3 comprising the amino acid sequence of SEQ ID NO: 21; and the VL domain comprises a CDR-L1 comprising the amino acid sequence of SEQ ID NO: 22, a CDR-L2 comprising the amino acid sequence of SEQ ID NO: 23, and a CDR-L3 comprising the amino acid sequence of SEQ ID NO: 24;
the VH domain comprises a CDR-H1 comprising the amino acid sequence of SEQ ID NO: 25, a CDR-H2 comprising the amino acid sequence of SEQ ID NO: 26, and a CDR-H3 comprising the amino acid sequence of SEQ ID NO: 27; and the VL domain comprises a CDR-L1 comprising the amino acid sequence of SEQ ID NO: 28, a CDR-L2 comprising the amino acid sequence of SEQ ID NO: 29, and a CDR-L3 comprising the amino acid sequence of SEQ ID NO: 30;
the VH domain comprises a CDR-H1 comprising the amino acid sequence of SEQ ID NO: 31, a CDR-H2 comprising the amino acid sequence of SEQ ID NO: 32, and a CDR-H3 comprising the amino acid sequence of SEQ ID NO: 33; and the VL domain comprises a CDR-L1 comprising the amino acid sequence of SEQ ID NO: 34, a CDR-L2 comprising the amino acid sequence of SEQ ID NO: 35, and a CDR-L3 comprising the amino acid sequence of SEQ ID NO: 36;
the VH domain comprises a CDR-H1 comprising the amino acid sequence of SEQ ID NO: 37, a CDR-H2 comprising the amino acid sequence of SEQ ID NO: 38, and a CDR-H3 comprising the amino acid sequence of SEQ ID NO: 39; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
the VH domain comprises a CDR-H1 comprising the amino acid sequence of SEQ ID NO: 43, a CDR-H2 comprising the amino acid sequence of SEQ ID NO: 44, and a CDR-H3 comprising the amino acid sequence of SEQ ID NO: 45; and the VL domain comprises a CDR-L1 comprising the amino acid sequence of SEQ ID NO: 46, a CDR-L2 comprising the amino acid sequence of SEQ ID NO: 47, and a CDR-L3 comprising the amino acid sequence of SEQ ID NO: 48;
the VH domain comprises a CDR-H1 comprising the amino acid sequence of SEQ ID NO: 1, a CDR-H2 comprising the amino acid sequence of SEQ ID NO: 2, and a CDR-H3 comprising the amino acid sequence of SEQ ID NO: 3; and the VL domain comprises a CDR-L1 comprising the amino acid sequence of SEQ ID NO: 4, a CDR-L2 comprising the amino acid sequence of SEQ ID NO: 5, and a CDR-L3 comprising the amino acid sequence of SEQ ID NO: 6;
the VH domain comprises a CDR-H1 comprising the amino acid sequence of SEQ ID NO: 177, a CDR-H2 comprising the amino acid sequence of SEQ ID NO: 178, and a CDR-H3 comprising the amino acid sequence of SEQ ID NO: 179; and the VL domain comprises a CDR-L1 comprising the amino acid sequence of SEQ ID NO: 180, a CDR-L2 comprising the amino acid sequence of SEQ ID NO: 181, and a CDR-L3 comprising the amino acid sequence of SEQ ID NO: 182;
the VH domain comprises a CDR-H1 comprising the amino acid sequence of SEQ ID NO: 225, a CDR-H2 comprising the amino acid sequence of SEQ ID NO: 226, and a CDR-H3 comprising the amino acid sequence of SEQ ID NO: 227; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228;
the VH domain comprises a CDR-H1 comprising the amino acid sequence of SEQ ID NO: 229, a CDR-H2 comprising the amino acid sequence of SEQ ID NO: 230, and a CDR-H3 comprising the amino acid sequence of SEQ ID NO: 231; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
the VH domain comprises a CDR-H1 comprising the amino acid sequence of SEQ ID NO: 225, a CDR-H2 comprising the amino acid sequence of SEQ ID NO: 232, and a CDR-H3 comprising the amino acid sequence of SEQ ID NO: 233; and the VL domain comprises a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236;
the VH domain comprises a CDR-H1 comprising the amino acid sequence of SEQ ID NO: 225, a CDR-H2 comprising the amino acid sequence of SEQ ID NO: 232, and a CDR-H3 comprising the amino acid sequence of SEQ ID NO: 233; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228; or
the VH domain comprises a CDR-H1 comprising the amino acid sequence of SEQ ID NO: 229, a CDR-H2 comprising the amino acid sequence of SEQ ID NO: 237, and a CDR-H3 comprising the amino acid sequence of SEQ ID NO: 231; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42.

Embodiment 18. The method of any one of Embodiments 1-16, wherein the first antibody or antigen-binding fragment thereof comprises a heavy chain and a light chain, wherein the heavy chain comprises a heavy chain variable (VH) domain, and the light chain comprises a light chain variable (VL) domain, and wherein in the first antibody or antigen-binding fragment thereof:
the VH domain comprises a CDR-H1 comprising the amino acid sequence of SEQ ID NO: 51, a CDR-H2 comprising the amino acid sequence of SEQ ID NO: 52, and a CDR-H3 comprising the amino acid sequence of SEQ ID NO: 15; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 18;
the VH domain comprises a CDR-H1 comprising the amino acid sequence of SEQ ID NO: 53, a CDR-H2 comprising the amino acid sequence of SEQ ID NO: 52, and a CDR-H3 comprising the amino acid sequence of SEQ ID NO: 21; and the VL domain comprises a CDR-L1 comprising the amino acid sequence of SEQ ID NO: 22, a CDR-L2 comprising the amino acid sequence of SEQ ID NO: 23, and a CDR-L3 comprising the amino acid sequence of SEQ ID NO: 24;
the VH domain comprises a CDR-H1 comprising the amino acid sequence of SEQ ID NO: 49, a CDR-H2 comprising the amino acid sequence of SEQ ID NO: 52, and a CDR-H3 comprising the amino acid sequence of SEQ ID NO: 27; and the VL domain comprises a CDR-L1 comprising the amino acid sequence of SEQ ID NO: 28, a CDR-L2 comprising the amino acid sequence of SEQ ID NO: 29, and a CDR-L3 comprising the amino acid sequence of SEQ ID NO: 30;
the VH domain comprises a CDR-H1 comprising the amino acid sequence of SEQ ID NO: 54, a CDR-H2 comprising the amino acid sequence of SEQ ID NO: 52, and a CDR-H3 comprising the amino acid sequence of SEQ ID NO: 33; and the VL domain comprises a CDR-L1 comprising the amino acid sequence of SEQ ID NO: 34, a CDR-L2 comprising the amino acid sequence of SEQ ID NO: 35, and a CDR-L3 comprising the amino acid sequence of SEQ ID NO: 36;
the VH domain comprises a CDR-H1 comprising the amino acid sequence of SEQ ID NO: 55, a CDR-H2 comprising the amino acid sequence of SEQ ID NO: 56, and a CDR-H3 comprising the amino acid sequence of SEQ ID NO: 39; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
the VH domain comprises a CDR-H1 comprising the amino acid sequence of SEQ ID NO: 55, a CDR-H2 comprising the amino acid sequence of SEQ ID NO: 57, and a CDR-H3 comprising the amino acid sequence of SEQ ID NO: 45; and the VL domain comprises a CDR-L1 comprising the amino acid sequence of SEQ ID NO: 46, a CDR-L2 comprising the amino acid sequence of SEQ ID NO: 47, and a CDR-L3 comprising the amino acid sequence of SEQ ID NO: 48;
the VH domain comprises a CDR-H1 comprising the amino acid sequence of SEQ ID NO: 49, a CDR-H2 comprising the amino acid sequence of SEQ ID NO: 50, and a CDR-H3 comprising the amino acid sequence of SEQ ID NO: 3; and the VL domain comprises a CDR-L1 comprising the amino acid sequence of SEQ ID NO: 4, a CDR-L2 comprising the amino acid sequence of SEQ ID NO: 5, and a CDR-L3 comprising the amino acid sequence of SEQ ID NO: 6;
the VH domain comprises a CDR-H1 comprising the amino acid sequence of SEQ ID NO: 183, a CDR-H2 comprising the amino acid sequence of SEQ ID NO: 184, and a CDR-H3 comprising the amino acid sequence of SEQ ID NO: 179; and the VL domain comprises a CDR-L1 comprising the amino acid sequence of SEQ ID NO: 180, a CDR-L2 comprising the amino acid sequence of SEQ ID NO: 181, and a CDR-L3 comprising the amino acid sequence of SEQ ID NO: 182;
the VH domain comprises a CDR-H1 comprising the amino acid sequence of SEQ ID NO: 238, a CDR-H2 comprising the amino acid sequence of SEQ ID NO: 239, and a CDR-H3 comprising the amino acid sequence of SEQ ID NO: 233; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228;
the VH domain comprises a CDR-H1 comprising the amino acid sequence of SEQ ID NO: 240, a CDR-H2 comprising the amino acid sequence of SEQ ID NO: 241, and a CDR-H3 comprising the amino acid sequence of SEQ ID NO: 242; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
the VH domain comprises a CDR-H1 comprising the amino acid sequence of SEQ ID NO: 238, a CDR-H2 comprising the amino acid sequence of SEQ ID NO: 243, and a CDR-H3 comprising the amino acid sequence of SEQ ID NO: 233; and the VL domain comprises a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236;
the VH domain comprises a CDR-H1 comprising the amino acid sequence of SEQ ID NO: 238, a CDR-H2 comprising the amino acid sequence of SEQ ID NO: 243, and a CDR-H3 comprising the amino acid sequence of SEQ ID NO: 233; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228; or
   the VH domain comprises a CDR-H1 comprising the amino acid sequence of SEQ ID NO: 240, a CDR-H2 comprising the amino acid sequence of SEQ ID NO: 244, and a CDR-H3 comprising the amino acid sequence of SEQ ID NO: 242; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42.

Embodiment 19. The method of Embodiment 17 or 18, wherein the VH and VL domains of the first antibody or antigen-binding fragment thereof comprise the amino acid sequences of:
(a) SEQ ID NOs: 58 and 59, respectively;
(b) SEQ ID NOs: 247 and 248, respectively;
(c) SEQ ID NOs: 62 and 63, respectively;
(d) SEQ ID NOs: 64 and 65, respectively;
(e) SEQ ID NOs: 66 and 67, respectively;
(f) SEQ ID NOs: 68 and 69, respectively;
(g) SEQ ID NOs: 70 and 71, respectively;
(h) SEQ ID NOs: 72 and 73, respectively;
(i) SEQ ID NOs: 185 and 186, respectively;
(j) SEQ ID NOs: 245 and 246, respectively;
(k) SEQ ID NOs: 249 and 250, respectively;
(l) SEQ ID NOs: 251 and 252, respectively;
(m) SEQ ID NOs: 253 and 254, respectively;
(n) SEQ ID NOs: 255 and 256, respectively; or
(o) SEQ ID NOs: 257 and 258, respectively.

Embodiment 20. The method of any one of Embodiments 1-19, wherein the second antibody or antigen-binding fragment thereof comprises a heavy chain and a light chain, wherein the heavy chain comprises a variable (VH) domain; and the light chain comprises a light chain variable (VL) domain, and wherein in the second antibody or antigen-binding fragment thereof:
the VH domain comprises a CDR-H1 comprising the amino acid sequence of SEQ ID NO: 13, a CDR-H2 comprising the amino acid sequence of SEQ ID NO: 14, and a CDR-H3 comprising the amino acid sequence of SEQ ID NO: 15; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 18;
the VH domain comprises a CDR-H1 comprising the amino acid sequence of SEQ ID NO: 19, a CDR-H2 comprising the amino acid sequence of SEQ ID NO: 20, and a CDR-H3 comprising the amino acid sequence of SEQ ID NO: 21; and the VL domain comprises a CDR-L1 comprising the amino acid sequence of SEQ ID NO: 22, a CDR-L2 comprising the amino acid sequence of SEQ ID NO: 23, and a CDR-L3 comprising the amino acid sequence of SEQ ID NO: 24;
the VH domain comprises a CDR-H1 comprising the amino acid sequence of SEQ ID NO: 25, a CDR-H2 comprising the amino acid sequence of SEQ ID NO: 26, and a CDR-H3 comprising the amino acid sequence of SEQ ID NO: 27; and the VL domain comprises a CDR-L1 comprising the amino acid sequence of SEQ ID NO: 28, a CDR-L2 comprising the amino acid sequence of SEQ ID NO: 29, and a CDR-L3 comprising the amino acid sequence of SEQ ID NO: 30;
the VH domain comprises a CDR-H1 comprising the amino acid sequence of SEQ ID NO: 31, a CDR-H2 comprising the amino acid sequence of SEQ ID NO: 32, and a CDR-H3 comprising the amino acid sequence of SEQ ID NO: 33; and the VL domain comprises a CDR-L1 comprising the amino acid sequence of SEQ ID NO: 34, a CDR-L2 comprising the amino acid sequence of SEQ ID NO: 35, and a CDR-L3 comprising the amino acid sequence of SEQ ID NO: 36;
the VH domain comprises a CDR-H1 comprising the amino acid sequence of SEQ ID NO: 37, a CDR-H2 comprising the amino acid sequence of SEQ ID NO: 38, and a CDR-H3 comprising the amino acid sequence of SEQ ID NO: 39; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
the VH domain comprises a CDR-H1 comprising the amino acid sequence of SEQ ID NO: 43, a CDR-H2 comprising the amino acid sequence of SEQ ID NO: 44, and a CDR-H3 comprising the amino acid sequence of SEQ ID NO: 45; and the VL domain comprises a CDR-L1 comprising the amino acid sequence of SEQ ID NO: 46, a CDR-L2 comprising the amino acid sequence of SEQ ID NO: 47, and a CDR-L3 comprising the amino acid sequence of SEQ ID NO: 48;
the VH domain comprises a CDR-H1 comprising the amino acid sequence of SEQ ID NO: 1, a CDR-H2 comprising the amino acid sequence of SEQ ID NO: 2, and a CDR-H3 comprising the amino acid sequence of SEQ ID NO: 3; and the VL domain comprises a CDR-L1 comprising the amino acid sequence of SEQ ID NO: 4, a CDR-L2 comprising the amino acid sequence of SEQ ID NO: 5, and a CDR-L3 comprising the amino acid sequence of SEQ ID NO: 6;
the VH domain comprises a CDR-H1 comprising the amino acid sequence of SEQ ID NO: 177, a CDR-H2 comprising the amino acid sequence of SEQ ID NO: 178, and a CDR-H3 comprising the amino acid sequence of SEQ ID NO: 179; and the VL domain comprises a CDR-L1 comprising the amino acid sequence of SEQ ID NO: 180, a CDR-L2 comprising the amino acid sequence of SEQ ID NO: 181, and a CDR-L3 comprising the amino acid sequence of SEQ ID NO: 182;
the VH domain comprises a CDR-H1 comprising the amino acid sequence of SEQ ID NO: 225, a CDR-H2 comprising the amino acid sequence of SEQ ID NO: 226, and a CDR-H3 comprising the amino acid sequence of SEQ ID NO: 227; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228;
the VH domain comprises a CDR-H1 comprising the amino acid sequence of SEQ ID NO: 229, a CDR-H2 comprising the amino acid sequence of SEQ ID NO: 230, and a CDR-H3 comprising the amino acid sequence of SEQ ID NO: 231; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
the VH domain comprises a CDR-H1 comprising the amino acid sequence of SEQ ID NO: 225, a CDR-H2 comprising the amino acid sequence of SEQ ID NO: 232, and a CDR-H3 comprising the amino acid sequence of SEQ ID NO: 233; and the VL domain comprises a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236;
the VH domain comprises a CDR-H1 comprising the amino acid sequence of SEQ ID NO: 225, a CDR-H2 comprising the amino acid sequence of SEQ ID NO: 232, and a CDR-H3 comprising the amino acid sequence of SEQ ID NO: 233; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228; or
the VH domain comprises a CDR-H1 comprising the amino acid sequence of SEQ ID NO: 229, a CDR-H2 comprising the amino acid sequence of SEQ ID NO: 237, and a CDR-H3 comprising the amino acid sequence of SEQ ID NO: 231; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42.

Embodiment 21. The method of any one of Embodiments 1-19, wherein the second antibody or antigen-binding fragment thereof comprises a heavy chain and a light chain, wherein the heavy chain comprises a heavy chain variable (VH) domain, and the light chain comprises a light chain variable (VL) domain, and wherein in the second antibody or antigen-binding fragment thereof:
the VH domain comprises a CDR-H1 comprising the amino acid sequence of SEQ ID NO: 51, a CDR-H2 comprising the amino acid sequence of SEQ ID NO: 52, and a CDR-H3 comprising the amino acid sequence of SEQ ID NO: 15; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 18;
the VH domain comprises a CDR-H1 comprising the amino acid sequence of SEQ ID NO: 53, a CDR-H2 comprising the amino acid sequence of SEQ ID NO: 52, and a CDR-H3 comprising the amino acid sequence of SEQ ID NO: 21; and the VL domain comprises a CDR-L1 comprising the amino acid sequence of SEQ ID NO: 22, a CDR-L2 comprising the amino acid sequence of SEQ ID NO: 23, and a CDR-L3 comprising the amino acid sequence of SEQ ID NO: 24;
the VH domain comprises a CDR-H1 comprising the amino acid sequence of SEQ ID NO: 49, a CDR-H2 comprising the amino acid sequence of SEQ ID NO: 52, and a CDR-H3 comprising the amino acid sequence of SEQ ID NO: 27; and the VL domain comprises a CDR-L1 comprising the amino acid sequence of SEQ ID NO: 28, a CDR-L2 comprising the amino acid sequence of SEQ ID NO: 29, and a CDR-L3 comprising the amino acid sequence of SEQ ID NO: 30;
the VH domain comprises a CDR-H1 comprising the amino acid sequence of SEQ ID NO: 54, a CDR-H2 comprising the amino acid sequence of SEQ ID NO: 52, and a CDR-H3 comprising the amino acid sequence of SEQ ID NO: 33; and the VL domain comprises a CDR-L1 comprising the amino acid sequence of SEQ ID NO: 34, a CDR-L2 comprising the amino acid sequence of SEQ ID NO: 35, and a CDR-L3 comprising the amino acid sequence of SEQ ID NO: 36;
the VH domain comprises a CDR-H1 comprising the amino acid sequence of SEQ ID NO: 55, a CDR-H2 comprising the amino acid sequence of SEQ ID NO: 56, and a CDR-H3 comprising the amino acid sequence of SEQ ID NO: 39; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
the VH domain comprises a CDR-H1 comprising the amino acid sequence of SEQ ID NO: 55, a CDR-H2 comprising the amino acid sequence of SEQ ID NO: 57, and a CDR-H3 comprising the amino acid sequence of SEQ ID NO: 45; and the VL domain comprises a CDR-L1 comprising the amino acid sequence of SEQ ID NO: 46, a CDR-L2 comprising the amino acid sequence of SEQ ID NO: 47, and a CDR-L3 comprising the amino acid sequence of SEQ ID NO: 48;
the VH domain comprises a CDR-H1 comprising the amino acid sequence of SEQ ID NO: 49, a CDR-H2 comprising the amino acid sequence of SEQ ID NO: 50, and a CDR-H3 comprising the amino acid sequence of SEQ ID NO: 3; and the VL domain comprises a CDR-L1 comprising the amino acid sequence of SEQ ID NO: 4, a CDR-L2 comprising the amino acid sequence of SEQ ID NO: 5, and a CDR-L3 comprising the amino acid sequence of SEQ ID NO: 6;
the VH domain comprises a CDR-H1 comprising the amino acid sequence of SEQ ID NO: 183, a CDR-H2 comprising the amino acid sequence of SEQ ID NO: 184, and a CDR-H3 comprising the amino acid sequence of SEQ ID NO: 179; and the VL domain comprises a CDR-L1 comprising the amino acid sequence of SEQ ID NO: 180, a CDR-L2 comprising the amino acid sequence of SEQ ID NO: 181, and a CDR-L3 comprising the amino acid sequence of SEQ ID NO: 182;
the VH domain comprises a CDR-H1 comprising the amino acid sequence of SEQ ID NO: 238, a CDR-H2 comprising the amino acid sequence of SEQ ID NO: 239, and a CDR-H3 comprising the amino acid sequence of SEQ ID NO: 233; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228;
the VH domain comprises a CDR-H1 comprising the amino acid sequence of SEQ ID NO: 240, a CDR-H2 comprising the amino acid sequence of SEQ ID NO: 241, and a CDR-H3 comprising the amino acid sequence of SEQ ID NO: 242; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
the VH domain comprises a CDR-H1 comprising the amino acid sequence of SEQ ID NO: 238, a CDR-H2 comprising the amino acid sequence of SEQ ID NO: 243, and a CDR-H3 comprising the amino acid sequence of SEQ ID NO: 233; and the VL domain comprises a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236;
the VH domain comprises a CDR-H1 comprising the amino acid sequence of SEQ ID NO: 238, a CDR-H2 comprising the amino acid sequence of SEQ ID NO: 243, and a CDR-H3 comprising the amino acid sequence of SEQ ID NO: 233; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228; or
the VH domain comprises a CDR-H1 comprising the amino acid sequence of SEQ ID NO: 240, a CDR-H2 comprising the amino acid sequence of SEQ ID NO: 244, and a CDR-H3 comprising the amino acid sequence of SEQ ID NO: 242; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42.

Embodiment 22. The method of Embodiment 20 or 21, wherein the VH and VL domains of the second antibody or antigen-binding fragment thereof comprise the amino acid sequences of:
(a) SEQ ID NOs: 58 and 59, respectively;
(b) SEQ ID NOs: 247 and 248, respectively;
(c) SEQ ID NOs: 62 and 63, respectively;
(d) SEQ ID NOs: 64 and 65, respectively;
(e) SEQ ID NOs: 66 and 67, respectively;
(f) SEQ ID NOs: 68 and 69, respectively;
(g) SEQ ID NOs: 70 and 71, respectively;
(h) SEQ ID NOs: 72 and 73, respectively;
(i) SEQ ID NOs: 185 and 186, respectively;
(j) SEQ ID NOs: 245 and 246, respectively;
(k) SEQ ID NOs: 249 and 250, respectively;
(l) SEQ ID NOs: 251 and 252, respectively;
(m) SEQ ID NOs: 253 and 254, respectively;
(n) SEQ ID NOs: 255 and 256, respectively; or
(o) SEQ ID NOs: 257 and 258, respectively.

Embodiment 23. The method of any one of Embodiments 1-22, wherein (i) the IL-21 polypeptide is linked to the first antibody or antigen binding fragment directly or by a linker, and/or (ii) the IL-2 polypeptide is linked to the second antibody or antigen binding fragment directly or by a linker.

Embodiment 24. The method of Embodiment 23, wherein (i) the IL-21 polypeptide is linked to the first antibody or antigen binding fragment by a Gly-Ser linker, and/or (ii) the IL-2 polypeptide is linked to the second antibody or antigen binding fragment by a Gly-Ser linker.

Embodiment 25. The method of any one of Embodiments 1-24, wherein the IL-2 fusion protein, the IL-21 fusion protein, or both the IL-2 fusion protein and IL-21 fusion protein each comprise:
two antibody heavy chain polypeptides comprising a structure according to formula [I], from N-terminus to C-terminus:

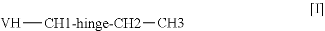

and two antibody light chain polypeptides comprising a structure according to formula [II], from N-terminus to C-terminus:

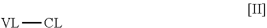

wherein VH is the VH domain, wherein CH1 is an antibody CH1 domain, wherein hinge is an antibody hinge domain, wherein CH2-CH3 is an antibody Fc domain, wherein VL is the VL domain, and wherein CL is an antibody constant light chain domain; and
wherein the N-terminus of the IL-2 polypeptide or the IL-21 polypeptide is fused to the C-terminus of one of the two CH3 domains.

Embodiment 26. The method of Embodiment 25, wherein one or both of the two antibody heavy chain polypeptides comprise(s) the following amino acid substitutions: L234A, L235A, and G237A, numbering according to EU index.

Embodiment 27. The method of Embodiment 25 or 26, wherein a first of the two antibody heavy chain polypeptides comprises amino acid substitutions Y349C and T366W, and a second of the two antibody heavy chain polypeptides comprises amino acid substitutions S354C, T366S, L368A and Y407V, numbering according to EU index.

Embodiment 28. The method of any one of Embodiments 1-25, wherein the IL-2 polypeptide has a binding affinity to IL-2Rα that is reduced by 50% or more, compared to binding affinity of a human IL-2 polypeptide comprising SEQ ID NO: 81.

Embodiment 29. The method of Embodiment 28, wherein the IL-2 polypeptide comprises one or more amino acid mutations relative to a human IL-2 polypeptide comprising SEQ ID NO: 81 that reduces the binding affinity of the IL-2 polypeptide.

Embodiment 30. The method of any one of Embodiments 1-29, wherein the IL-2 polypeptide comprises the amino acid sequence of SEQ ID NO: 81 with one, two, three, four, or five amino acid substitutions relative to SEQ ID NO: 81, and wherein the one, two, three, four, or five substitution(s) comprise substitution(s) at positions of SEQ ID NO: 81 selected from the group consisting of: Q11, H16, L18, L19, D20, Q22, R38, F42, K43, Y45, E62, P65, E68, V69, L72, D84, S87, N88, V91, 192, T123, Q126, S127, 1129, and S130.

Embodiment 31. The method of Embodiment 30, wherein the IL-2 polypeptide comprises the sequence of SEQ ID NO: 81 with a set of amino acid substitutions relative to the sequence of SEQ ID NO: 81 selected from the group consisting of: R38E and F42A; R38D and F42A; F42A and E62Q; R38A and F42K; R38E, F42A, and N88S; R38E, F42A, and N88A; R38E, F42A, and N88G; R38E, F42A, and N88R; R38E, F42A, and N88T; R38E, F42A, and N88D; R38E, F42A, and V91E; R38E, F42A, and D84H; R38E, F42A, and D84K; R38E, F42A, and D84R; H16D, R38E and F42A; H16E, R38E and F42A; R38E, F42A and Q126S; R38D, F42A and N88S; R38D, F42A and N88A; R38D, F42A and N88G; R38D, F42A and N88R; R38D, F42A and N88T; R38D, F42A and N88D; R38D, F42A and V91E; R38D, F42A, and D84H; R38D, F42A, and D84K; R38D, F42A, and D84R; H16D, R38D and F42A; H16E, R38D and F42A; R38D, F42A and Q126S; R38A, F42K, and N88S; R38A, F42K, and N88A; R38A, F42K, and N88G; R38A, F42K, and N88R; R38A, F42K, and N88T; R38A, F42K, and N88D; R38A, F42K, and V91E; R38A, F42K, and D84H; R38A, F42K, and D84K; R38A, F42K, and D84R; H16D, R38A, and F42K; H16E, R38A, and F42K; R38A, F42K, and Q126S; F42A, E62Q, and N88S; F42A, E62Q, and N88A; F42A, E62Q, and N88G; F42A, E62Q, and N88R; F42A, E62Q, and N88T; F42A, E62Q, and N88D; F42A, E62Q, and V91E; F42A, E62Q, and D84H; F42A, E62Q, and D84K; and F42A, E62Q, and D84R.

Embodiment 32. The method of Embodiment 30, wherein the IL-2 polypeptide comprises the sequence of SEQ ID NO: 81 with a further amino acid substitution relative to SEQ ID NO: 81 at position C125.

Embodiment 33. The method of Embodiment 32, wherein the IL-2 polypeptide comprises the sequence of SEQ ID NO: 81 with a set of amino acid substitutions relative to the sequence of SEQ ID NO: 81 selected from the group consisting of: R38E, F42A, and C125A; R38D, F42A, and C125A; F42A, E62Q, and C125A; R38A, F42K, and C125A; R38E, F42A, N88S, and C125A; R38E, F42A, N88A, and C125A; R38E, F42A, N88G, and C125A; R38E, F42A, N88R, and C125A; R38E, F42A, N88D, and C125A; R38E, F42A, N88T, and C125A; R38E, F42A, V91E, and C125A; R38E, F42A, D84H, and C125A; R38E, F42A, D84K, and C125A; R38E, F42A, D84R, and C125A; H16D, R38E, F42A, and C125A; H16E, R38E, F42A, and C125A; R38E, F42A, C125A and Q126S; R38D, F42A, N88S, and C125A; R38D, F42A, N88A, and C125A; R38D, F42A, N88G, and C125A; R38D, F42A, N88R, and C125A; R38D, F42A, N88T, and C125A; R38D, F42A, N88D, and C125A; R38D, F42A, V91E, and C125A; R38D, F42A, D84H, and C125A; R38D, F42A, D84K, and C125A; R38D, F42A, D84R, and C125A; H16D, R38D, F42A, and C125A; H16E, R38D, F42A, and C125A; R38D, F42A, C125A, and Q126S; R38A, F42K, N88S, and C125A; R38A, F42K, N88G, and C125A; R38A, F42K, N88R, and C125A; R38A, F42K, N88T, and C125A; R38A, F42K, N88D, and C125A; R38A, F42K, N88A, and C125A; R38A, F42K, V91E, and C125A; R38A, F42K, D84H, and C125A; R38A, F42K, D84K, and C125A; R38A, F42K, D84R, and C125A; H16D, R38A, F42K, and C125A; H16E, R38A, F42K, and C125A; R38A, F42K, C125A and Q126S; F42A, E62Q, N88S, and C125A; F42A, E62Q, N88A, and C125A; F42A, E62Q, N88G, and C125A; F42A, E62Q, N88R, and C125A; F42A, E62Q, N88T, and C125A; F42A, E62Q, N88D, and C125A; F42A, E62Q, V91E, and C125A; F42A, E62Q, and D84H, and C125A; F42A, E62Q, and D84K, and C125A; F42A, E62Q, and D84R, and C125A; H16D, F42A, and E62Q, and C125A; H16E, F42A, E62Q, and C125A; F42A, E62Q, C125A and Q126S; F42A, N88S, and C125A; F42A, N88A, and C125A; F42A, N88G, and C125A; F42A, N88R, and C125A; F42A, N88T, and C125A; F42A, N88D, and C125A; F42A, V91E, and C125A; F42A, D84H, and C125A; F42A, D84K, and C125A; F42A, D84R, and C125A; H16D, F42A, and C125A; H16E, F42A, and C125A; and F42A, C125A and Q126S.

Embodiment 34. The method of any one of Embodiments 1-33, wherein the IL-2 polypeptide comprises the amino acid sequence of SEQ ID NO: 80 or SEQ ID NO: 297.

Embodiment 35. The method of any one of Embodiments 1-33, wherein the IL-2 polypeptide comprises a sequence selected from the group consisting of SEQ ID NOs: 80, 85-155, 190-216, 297, and 354-383.

Embodiment 36. The method of any one of Embodiments 1-35, wherein the IL-21 polypeptide comprises an amino acid sequence that is at least 80% identical to a human IL-21 polypeptide comprising SEQ ID NO: 390.

Embodiment 37. The method of Embodiment 36, wherein the IL-21 polypeptide has an isoelectric point that is at least about 0.6 units to about 5 units lower, compared to that of the human IL-21 polypeptide.

Embodiment 38. The method of Embodiment 37, wherein the IL-21 polypeptide comprises at least one amino acid substitution that reduces the isoelectric point of the IL-21 polypeptide by about 0.6 units to about 5 units relative to the human IL-21 polypeptide without the amino acid substitution.

Embodiment 39. The method of Embodiment 38, wherein the IL-21 polypeptide comprises at least four amino acid substitutions that reduce the isoelectric point of the IL-21 polypeptide by about 0.6 units to about 5 units relative to the human IL-21 polypeptide without the amino acid substitutions.

Embodiment 40. The method of Embodiment 38 or 39, wherein the IL-21 polypeptide comprises up to 10, 11, 12, 13, 14, 15, 16, 17, 18, 19 or 20 amino acid substitutions that reduce the isoelectric point.

Embodiment 41. The method of any one of Embodiments 36-40, wherein the human IL-21 polypeptide has an isoelectric point of about 9.42.

Embodiment 42. The method of any one of Embodiments 36-41, wherein the IL-21 polypeptide has an isoelectric point of about 7.12 to about 8.72.

Embodiment 43. The method of any one of Embodiments 36-42, wherein the IL-21 polypeptide results in an improved exposure following administration to the subject relative to the human IL-21 polypeptide, as measured by at least about 1.5 times greater area under the curve (AUC).

Embodiment 44. The method of any one of Embodiments 37-40, wherein the IL-21 polypeptide comprises a modified region, wherein relative to a region of the human IL-21 polypeptide that comprises about 2 to 20 positively charged amino acid residues, the modified region comprises one or more amino acid substitutions of the about 2 to 20 positively charged amino acid residues.

Embodiment 45. The method of Embodiment 44, wherein the modified region excludes amino acid residues that bind a human IL-21 receptor.

Embodiment 46. The method of Embodiment 44 or 45, wherein the modified region comprises amino acid residues positioned at S80 to T92 of the human IL-21 polypeptide comprising SEQ ID NO. 390.

Embodiment 47. The method of any one of Embodiments 44-46, wherein the human IL-21 polypeptide comprises at least one positively charged amino acid residue on the surface of the human IL-21 polypeptide in a three-dimensional structure of the human IL-21 polypeptide and does not bind an IL-21 receptor, and wherein the IL-21 polypeptide comprises at least one amino acid substitution of at the least one positively charged amino acid residue.

Embodiment 48. The method of any one of Embodiments 44-47, wherein the IL-21 polypeptide does not comprise an amino acid substitution at G84 of the human IL-21 polypeptide comprising SEQ ID NO: 390.

Embodiment 49. The method of any one of Embodiments 37-48, wherein the IL-21 polypeptide comprises 4, 5, 6, 7, 8, 9, 10, 11, or 12 amino acid substitutions at positions relative to SEQ ID NO: 81 selected from the group consisting of: S80, T81, N82, A83, R85, R86, Q87, K88, H89, R90, L91, and T92 of SEQ ID NO: 390.

Embodiment 50. The method of Embodiment 49, wherein the IL-21 polypeptide comprises 4, 5, 6, 7, 8, 9, 10, 11, or 12 amino acid substitutions selected from the group consisting of: S80G, T81G, N82G, N82E, A83G, A83E, A83S, R85G, R85E, R85S, R86G, R86E, Q87G, Q87E, Q87S, K88G, H89G, H89S, R90G, R90S, R90E, R90A, L91G, L91S, T92G, and T92S.

Embodiment 51. The method of Embodiment 50, wherein the IL-21 polypeptide comprises (a) R85G, R86G, K88G and R90E, or (b) S80G, T81G, N82E, A83G, R85G, R86G, Q87G, K88G, H89G, R90E, L91G, and T92G.

Embodiment 52. The method of any one of Embodiments 1-51, wherein the IL-21 polypeptide comprises the amino acid sequence: QGQDRHMIRMRQLIDIVDQLKNYVNDLVPEFLPAPEDVETNCEWSAFSCFQKAQLK SANTGNNERIINVSIKKLKRKPPX₁X₂X₃X₄GX₅X₆X₇X₈X₉X₁₀X₁₁X₁₂CPSCDSYEKKPPK EFLERFKSLLQKMIHQHLSSRTHGSEDS (SEQ ID NO: 554), wherein X₁=G, S; X₂=G, T; X₃=G, E, N; X₄=G, S, E, A; X₅=G, E, S, R; X₆=G, E, R; X₇=S, G, E, Q; X₈=G, K; X₉=G, S, H; X₁₀=A, E, S, G, R; X₁₁=S, G, L; and X₁₂=G, S, T, provided at least one of X₅, X₆, X₈, X₉, and X₁₀ is not the amino acid residue at the identical position set forth in SEQ ID NO: 390, optionally wherein:

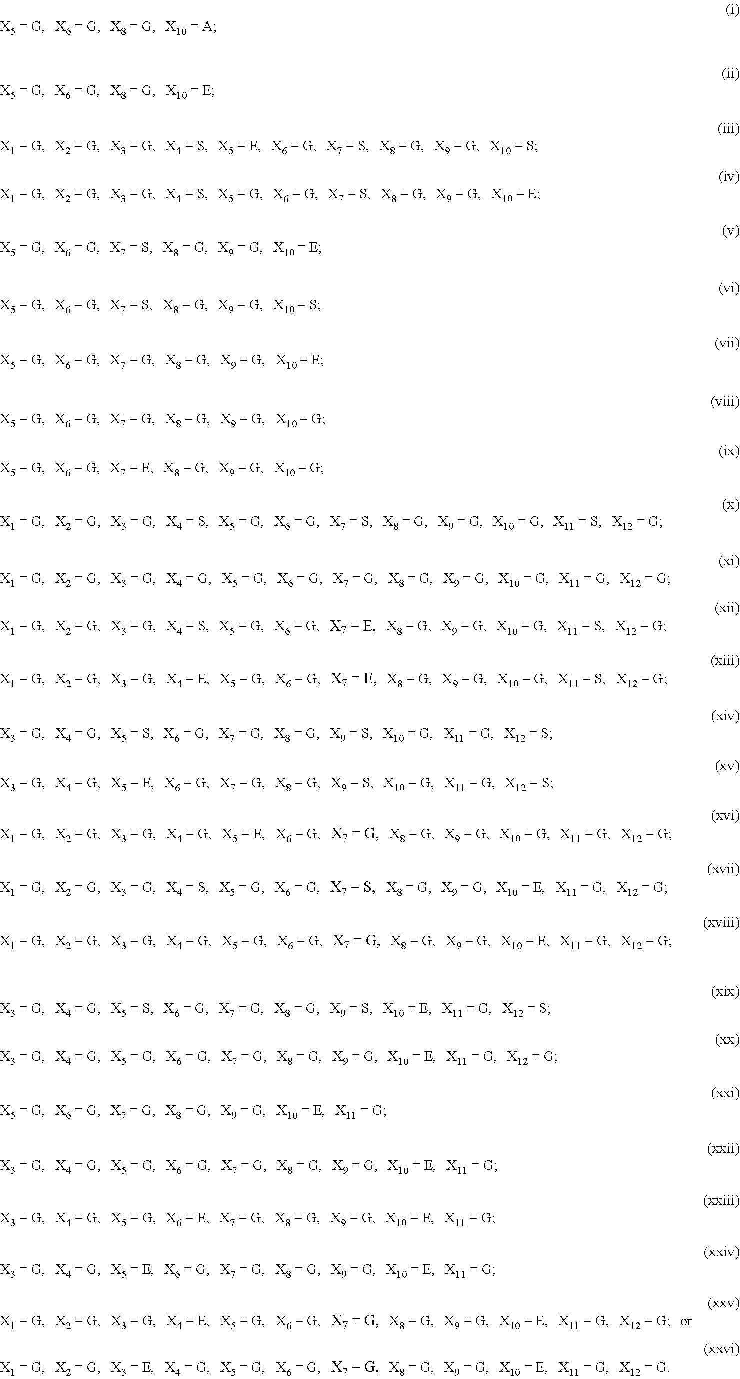

Embodiment 53. The method of any one of Embodiments 1-52, wherein the IL-12 polypeptide comprises an amino acid sequence at least 90% identical to an amino acid sequence selected from SEQ ID NOs: 391-421.

Embodiment 54. The method of Embodiment 53, wherein the IL-21 polypeptide comprises an amino acid sequence selected from SEQ ID NOs: 391-421.

Embodiment 55. The fusion protein of Embodiment 54, wherein the IL-21 polypeptide comprises the amino acid sequence of SEQ ID NO: 421.

Embodiment 56. The method of any one of Embodiments 1-55, wherein the IL-21 polypeptide comprises at least one amino acid substitution that reduces binding to an IL-21 receptor compared to binding by the human IL-21 polypeptide.

Embodiment 57. The method of Embodiment 56, wherein the at least one amino acid substitution is at one or more amino acid residues at positions R5, I8, R9, R11, L13, I14, I16, V17, D18, K72, K73, L74, K75, R76, K77, or K117, of SEQ ID NO: 390.

Embodiment 58. The method of Embodiment 57, wherein the at least one amino acid substitution is selected from: R5F, R5A, R5E, R5S, R5T, R5N, R5Q, R5V, R5I, R5L, R5Y, 18E, R9A, R9D, R9E, R9H, R9S, R9T, R9N, R9G, R9V, R9I, R9L, R9Y, RI1D, RI1E, L13F, L13R, 114D, 116A, 116S, 116R, V171, V17A, D18A, K72A, K72E, K73A, K73E, K75A, K75E, L74I, L74F, L74M, L74V, R76E, R76F, R76A, R76N, R76D, R76S, R76T, R76Q, R76V, R76I, R76L, R76Y, R76M, K77A, K77E, and K117A.

Embodiment 59. The method of Embodiment 57 or 58, wherein the at least one amino acid substitution is R76E or R76Q.

Embodiment 60. The method of any one of Embodiments 1-59, wherein the IL-21 polypeptide comprises an amino acid sequence at least 90% identical to an amino acid sequence selected from SEQ ID NOs: 422-491.

Embodiment 61. The method of Embodiment 60, wherein the IL-21 polypeptide comprises an amino acid sequence selected from SEQ ID NOs: 422-491.

Embodiment 62. The method of any one of Embodiments 1-61, wherein the IL-2 fusion protein comprises:
one or two light chains comprising the amino acid sequence of SEQ ID NO: 156, a heavy chain comprising the amino acid sequence of SEQ ID NO: 157, and a heavy chain comprising the amino acid sequence of SEQ ID NO: 158;
one or two light chains comprising the amino acid sequence of SEQ ID NO: 159, a heavy chain comprising the amino acid sequence of SEQ ID NO: 160, and a heavy chain comprising the amino acid sequence of SEQ ID NO: 161;
one or two light chains comprising the amino acid sequence of SEQ ID NO: 162, a heavy chain comprising the amino acid sequence of SEQ ID NO: 163, and a heavy chain comprising the amino acid sequence of SEQ ID NO: 164;
one or two light chains comprising the amino acid sequence of SEQ ID NO: 165, a heavy chain comprising the amino acid sequence of SEQ ID NO: 166, and a heavy chain comprising the amino acid sequence of SEQ ID NO: 167;
one or two light chains comprising the amino acid sequence of SEQ ID NO: 168, a heavy chain comprising the amino acid sequence of SEQ ID NO: 169, and a heavy chain comprising the amino acid sequence of SEQ ID NO: 170;
one or two light chains comprising the amino acid sequence of SEQ ID NO: 171, a heavy chain comprising the amino acid sequence of SEQ ID NO: 172, and a heavy chain comprising the amino acid sequence of SEQ ID NO: 173;
one or two light chains comprising the amino acid sequence of SEQ ID NO: 174, a heavy chain comprising the amino acid sequence of SEQ ID NO: 175, and a heavy chain comprising the amino acid sequence of SEQ ID NO: 176;
one or two light chains comprising the amino acid sequence of SEQ ID NO: 187, a heavy chain comprising the amino acid sequence of SEQ ID NO: 188, and a heavy chain comprising the amino acid sequence of SEQ ID NO: 189;
one or two light chains comprising the amino acid sequence of SEQ ID NO: 298, a heavy chain comprising the amino acid sequence of SEQ ID NO: 299, and a heavy chain comprising the amino acid sequence of SEQ ID NO: 300;
one or two light chains comprising the amino acid sequence of SEQ ID NO: 302, a heavy chain comprising the amino acid sequence of SEQ ID NO: 303, and a heavy chain comprising the amino acid sequence of SEQ ID NO: 304;
one or two light chains comprising the amino acid sequence of SEQ ID NO: 306, a heavy chain comprising the amino acid sequence of SEQ ID NO: 307, and a heavy chain comprising the amino acid sequence of SEQ ID NO: 308;
one or two light chains comprising the amino acid sequence of SEQ ID NO: 310, a heavy chain comprising the amino acid sequence of SEQ ID NO: 311, and a heavy chain comprising the amino acid sequence of SEQ ID NO: 312;
one or two light chains comprising the amino acid sequence of SEQ ID NO: 314, a heavy chain comprising the amino acid sequence of SEQ ID NO: 315, and a heavy chain comprising the amino acid sequence of SEQ ID NO: 316;
one or two light chains comprising the amino acid sequence of SEQ ID NO: 318, a heavy chain comprising the amino acid sequence of SEQ ID NO: 319, and a heavy chain comprising the amino acid sequence of SEQ ID NO: 320;
one or two light chains comprising the amino acid sequence of SEQ ID NO: 322, a heavy chain comprising the amino acid sequence of SEQ ID NO: 323, and a heavy chain comprising the amino acid sequence of SEQ ID NO: 324;
one or two light chains comprising the amino acid sequence of SEQ ID NO: 326, a heavy chain comprising the amino acid sequence of SEQ ID NO: 327, and a heavy chain comprising the amino acid sequence of SEQ ID NO: 328;
one or two light chains comprising the amino acid sequence of SEQ ID NO: 330, a heavy chain comprising the amino acid sequence of SEQ ID NO: 331, and a heavy chain comprising the amino acid sequence of SEQ ID NO: 332;
one or two light chains comprising the amino acid sequence of SEQ ID NO: 334, a heavy chain comprising the amino acid sequence of SEQ ID NO: 335, and a heavy chain comprising the amino acid sequence of SEQ ID NO: 336;
one or two light chains comprising the amino acid sequence of SEQ ID NO: 338, a heavy chain comprising the amino acid sequence of SEQ ID NO: 339, and a heavy chain comprising the amino acid sequence of SEQ ID NO: 340;
one or two light chains comprising the amino acid sequence of SEQ ID NO: 342, a heavy chain comprising the amino acid sequence of SEQ ID NO: 343, and a heavy chain comprising the amino acid sequence of SEQ ID NO: 344;
one or two light chains comprising the amino acid sequence of SEQ ID NO: 346, a heavy chain comprising the amino acid sequence of SEQ ID NO: 347, and a heavy chain comprising the amino acid sequence of SEQ ID NO: 348;
one or two light chains comprising the amino acid sequence of SEQ ID NO: 350, a heavy chain comprising the amino acid sequence of SEQ ID NO: 351, and a heavy chain comprising the amino acid sequence of SEQ ID NO: 352;
one or two light chains comprising the amino acid sequence of SEQ ID NO: 156, a heavy chain comprising the amino acid sequence of SEQ ID NO: 157, and a heavy chain comprising the amino acid sequence of SEQ ID NO: 217;
one or two light chains comprising the amino acid sequence of SEQ ID NO: 159, a heavy chain comprising the amino acid sequence of SEQ ID NO: 160, and a heavy chain comprising the amino acid sequence of SEQ ID NO: 218;
one or two light chains comprising the amino acid sequence of SEQ ID NO: 162, a heavy chain comprising the amino acid sequence of SEQ ID NO: 163, and a heavy chain comprising the amino acid sequence of SEQ ID NO: 219;
one or two light chains comprising the amino acid sequence of SEQ ID NO: 165, a heavy chain comprising the amino acid sequence of SEQ ID NO: 166, and a heavy chain comprising the amino acid sequence of SEQ ID NO: 220;
one or two light chains comprising the amino acid sequence of SEQ ID NO: 168, a heavy chain comprising the amino acid sequence of SEQ ID NO: 169, and a heavy chain comprising the amino acid sequence of SEQ ID NO: 221;
one or two light chains comprising the amino acid sequence of SEQ ID NO: 171, a heavy chain comprising the amino acid sequence of SEQ ID NO: 172, and a heavy chain comprising the amino acid sequence of SEQ ID NO: 222;
one or two light chains comprising the amino acid sequence of SEQ ID NO: 174, a heavy chain comprising the amino acid sequence of SEQ ID NO: 175, and a heavy chain comprising the amino acid sequence of SEQ ID NO: 223;
one or two light chains comprising the amino acid sequence of SEQ ID NO: 187, a heavy chain comprising the amino acid sequence of SEQ ID NO: 188, and a heavy chain comprising the amino acid sequence of SEQ ID NO: 224;
one or two light chains comprising the amino acid sequence of SEQ ID NO: 298, a heavy chain comprising the amino acid sequence of SEQ ID NO: 299, and a heavy chain comprising the amino acid sequence of SEQ ID NO: 301;
one or two light chains comprising the amino acid sequence of SEQ ID NO: 302, a heavy chain comprising the amino acid sequence of SEQ ID NO: 303, and a heavy chain comprising the amino acid sequence of SEQ ID NO: 305;
one or two light chains comprising the amino acid sequence of SEQ ID NO: 306, a heavy chain comprising the amino acid sequence of SEQ ID NO: 307, and a heavy chain comprising the amino acid sequence of SEQ ID NO: 309;
one or two light chains comprising the amino acid sequence of SEQ ID NO: 310, a heavy chain comprising the amino acid sequence of SEQ ID NO: 311, and a heavy chain comprising the amino acid sequence of SEQ ID NO: 313;
one or two light chains comprising the amino acid sequence of SEQ ID NO: 314, a heavy chain comprising the amino acid sequence of SEQ ID NO: 315, and a heavy chain comprising the amino acid sequence of SEQ ID NO: 317;
one or two light chains comprising the amino acid sequence of SEQ ID NO: 318, a heavy chain comprising the amino acid sequence of SEQ ID NO: 319, and a heavy chain comprising the amino acid sequence of SEQ ID NO: 321;
one or two light chains comprising the amino acid sequence of SEQ ID NO: 322, a heavy chain comprising the amino acid sequence of SEQ ID NO: 323, and a heavy chain comprising the amino acid sequence of SEQ ID NO: 325;
one or two light chains comprising the amino acid sequence of SEQ ID NO: 326, a heavy chain comprising the amino acid sequence of SEQ ID NO: 327, and a heavy chain comprising the amino acid sequence of SEQ ID NO: 329;
one or two light chains comprising the amino acid sequence of SEQ ID NO: 330, a heavy chain comprising the amino acid sequence of SEQ ID NO: 331, and a heavy chain comprising the amino acid sequence of SEQ ID NO: 333;
one or two light chains comprising the amino acid sequence of SEQ ID NO: 334, a heavy chain comprising the amino acid sequence of SEQ ID NO: 335, and a heavy chain comprising the amino acid sequence of SEQ ID NO: 337;
one or two light chains comprising the amino acid sequence of SEQ ID NO: 338, a heavy chain comprising the amino acid sequence of SEQ ID NO: 339, and a heavy chain comprising the amino acid sequence of SEQ ID NO: 341;
one or two light chains comprising the amino acid sequence of SEQ ID NO: 342, a heavy chain comprising the amino acid sequence of SEQ ID NO: 343, and a heavy chain comprising the amino acid sequence of SEQ ID NO: 345;
one or two light chains comprising the amino acid sequence of SEQ ID NO: 346, a heavy chain comprising the amino acid sequence of SEQ ID NO: 347, and a heavy chain comprising the amino acid sequence of SEQ ID NO: 349; or
one or two light chains comprising the amino acid sequence of SEQ ID NO: 350, a heavy chain comprising the amino acid sequence of SEQ ID NO: 351, and a heavy chain comprising the amino acid sequence of SEQ ID NO: 353.

Embodiment 63. The method of any one of Embodiments 1-62, wherein the IL-2 fusion protein comprises four polypeptide chains, wherein:
the first polypeptide chain comprises the amino acid sequence of SEQ ID NO: 334, the second polypeptide chain comprises the amino acid sequence of SEQ ID NO: 335, the third polypeptide chain comprises the amino acid sequence of SEQ ID NO: 336, and the fourth polypeptide chain comprises the amino acid sequence of SEQ ID NO: 334;
the first polypeptide chain comprises the amino acid sequence of SEQ ID NO: 334, the second polypeptide chain comprises the amino acid sequence of SEQ ID NO: 335, the third polypeptide chain comprises the amino acid sequence of SEQ ID NO: 337, and the fourth polypeptide chain comprises the amino acid sequence of SEQ ID NO: 334;
the first polypeptide chain comprises the amino acid sequence of SEQ ID NO: 338, the second polypeptide chain comprises the amino acid sequence of SEQ ID NO: 339, the third polypeptide chain comprises the amino acid sequence of SEQ ID NO: 340, and the fourth polypeptide chain comprises the amino acid sequence of SEQ ID NO: 338; or
the first polypeptide chain comprises the amino acid sequence of SEQ ID NO: 338, the second polypeptide chain comprises the amino acid sequence of SEQ ID NO: 339, the third polypeptide chain comprises the amino acid sequence of SEQ ID NO: 341, and the fourth polypeptide chain comprises the amino acid sequence of SEQ ID NO: 338.

Embodiment 64. The method of any one of Embodiments 1-63, wherein the IL-21 fusion protein comprises:
one or two light chains comprising the amino acid sequence of SEQ ID NO: 557, a heavy chain comprising the amino acid sequence of SEQ ID NO: 558, and a heavy chain comprising the amino acid sequence of SEQ ID NO: 559;
one or two light chains comprising the amino acid sequence of SEQ ID NO: 561, a heavy chain comprising the amino acid sequence of SEQ ID NO: 562, and a heavy chain comprising the amino acid sequence of SEQ ID NO: 563;
one or two light chains comprising the amino acid sequence of SEQ ID NO: 565, a heavy chain comprising the amino acid sequence of SEQ ID NO: 566, and a heavy chain comprising the amino acid sequence of SEQ ID NO: 567;
one or two light chains comprising the amino acid sequence of SEQ ID NO: 569, a heavy chain comprising the amino acid sequence of SEQ ID NO: 570, and a heavy chain comprising the amino acid sequence of SEQ ID NO: 571;
one or two light chains comprising the amino acid sequence of SEQ ID NO: 573, a heavy chain comprising the amino acid sequence of SEQ ID NO: 574, and a heavy chain comprising the amino acid sequence of SEQ ID NO: 575;
one or two light chains comprising the amino acid sequence of SEQ ID NO: 577, a heavy chain comprising the amino acid sequence of SEQ ID NO: 578, and a heavy chain comprising the amino acid sequence of SEQ ID NO: 579;
one or two light chains comprising the amino acid sequence of SEQ ID NO: 581, a heavy chain comprising the amino acid sequence of SEQ ID NO: 582, and a heavy chain comprising the amino acid sequence of SEQ ID NO: 583;
one or two light chains comprising the amino acid sequence of SEQ ID NO: 585, a heavy chain comprising the amino acid sequence of SEQ ID NO: 586, and a heavy chain comprising the amino acid sequence of SEQ ID NO: 587;
one or two light chains comprising the amino acid sequence of SEQ ID NO: 589, a heavy chain comprising the amino acid sequence of SEQ ID NO: 590, and a heavy chain comprising the amino acid sequence of SEQ ID NO: 591;
one or two light chains comprising the amino acid sequence of SEQ ID NO: 557, a heavy chain comprising the amino acid sequence of SEQ ID NO: 558, and a heavy chain comprising the amino acid sequence of SEQ ID NO: 560;
one or two light chains comprising the amino acid sequence of SEQ ID NO: 561, a heavy chain comprising the amino acid sequence of SEQ ID NO: 562, and a heavy chain comprising the amino acid sequence of SEQ ID NO: 564;
one or two light chains comprising the amino acid sequence of SEQ ID NO: 565, a heavy chain comprising the amino acid sequence of SEQ ID NO: 566, and a heavy chain comprising the amino acid sequence of SEQ ID NO: 568;
one or two light chains comprising the amino acid sequence of SEQ ID NO: 569, a heavy chain comprising the amino acid sequence of SEQ ID NO: 570, and a heavy chain comprising the amino acid sequence of SEQ ID NO: 572;
one or two light chains comprising the amino acid sequence of SEQ ID NO: 573, a heavy chain comprising the amino acid sequence of SEQ ID NO: 574, and a heavy chain comprising the amino acid sequence of SEQ ID NO: 576;
one or two light chains comprising the amino acid sequence of SEQ ID NO: 577, a heavy chain comprising the amino acid sequence of SEQ ID NO: 578, and a heavy chain comprising the amino acid sequence of SEQ ID NO: 580;
one or two light chains comprising the amino acid sequence of SEQ ID NO: 581, a heavy chain comprising the amino acid sequence of SEQ ID NO: 582, and a heavy chain comprising the amino acid sequence of SEQ ID NO: 584;
one or two light chains comprising the amino acid sequence of SEQ ID NO: 585, a heavy chain comprising the amino acid sequence of SEQ ID NO: 586, and a heavy chain comprising the amino acid sequence of SEQ ID NO: 588; or
one or two light chains comprising the amino acid sequence of SEQ ID NO: 589, a heavy chain comprising the amino acid sequence of SEQ ID NO: 590, and a heavy chain comprising the amino acid sequence of SEQ ID NO: 592.

Embodiment 65. The method of any one of Embodiments 1-64, wherein the IL-21 fusion protein comprises four polypeptide chains, wherein:
the first polypeptide chain comprises the amino acid sequence of SEQ ID NO: 557, the second polypeptide chain comprises the amino acid sequence of SEQ ID NO: 558, the third polypeptide chain comprises the amino acid sequence of SEQ ID NO: 559, and the fourth polypeptide chain comprises the amino acid sequence of SEQ ID NO: 557;
the first polypeptide chain comprises the amino acid sequence of SEQ ID NO: 557, the second polypeptide chain comprises the amino acid sequence of SEQ ID NO: 558, the third polypeptide chain comprises the amino acid sequence of SEQ ID NO: 560, and the fourth polypeptide chain comprises the amino acid sequence of SEQ ID NO: 557;
the first polypeptide chain comprises the amino acid sequence of SEQ ID NO: 561, the second polypeptide chain comprises the amino acid sequence of SEQ ID NO: 562, the third polypeptide chain comprises the amino acid sequence of SEQ ID NO: 563, and the fourth polypeptide chain comprises the amino acid sequence of SEQ ID NO: 561;
the first polypeptide chain comprises the amino acid sequence of SEQ ID NO: 561, the second polypeptide chain comprises the amino acid sequence of SEQ ID NO: 562, the third polypeptide chain comprises the amino acid sequence of SEQ ID NO: 564, and the fourth polypeptide chain comprises the amino acid sequence of SEQ ID NO: 561;
the first polypeptide chain comprises the amino acid sequence of SEQ ID NO: 565, the second polypeptide chain comprises the amino acid sequence of SEQ ID NO: 566, the third polypeptide chain comprises the amino acid sequence of SEQ ID NO: 567, and the fourth polypeptide chain comprises the amino acid sequence of SEQ ID NO: 565;
the first polypeptide chain comprises the amino acid sequence of SEQ ID NO: 565, the second polypeptide chain comprises the amino acid sequence of SEQ ID NO: 566, the third polypeptide chain comprises the amino acid sequence of SEQ ID NO: 568, and the fourth polypeptide chain comprises the amino acid sequence of SEQ ID NO: 565;
the first polypeptide chain comprises the amino acid sequence of SEQ ID NO: 569, the second polypeptide chain comprises the amino acid sequence of SEQ ID NO: 570, the third polypeptide chain comprises the amino acid sequence of SEQ ID NO: 571, and the fourth polypeptide chain comprises the amino acid sequence of SEQ ID NO: 569;
the first polypeptide chain comprises the amino acid sequence of SEQ ID NO: 569, the second polypeptide chain comprises the amino acid sequence of SEQ ID NO: 570, the third polypeptide chain comprises the amino acid sequence of SEQ ID NO: 572, and the fourth polypeptide chain comprises the amino acid sequence of SEQ ID NO: 569;
the first polypeptide chain comprises the amino acid sequence of SEQ ID NO: 573, the second polypeptide chain comprises the amino acid sequence of SEQ ID NO: 574, the third polypeptide chain comprises the amino acid sequence of SEQ ID NO: 575, and the fourth polypeptide chain comprises the amino acid sequence of SEQ ID NO: 573;
the first polypeptide chain comprises the amino acid sequence of SEQ ID NO: 573, the second polypeptide chain comprises the amino acid sequence of SEQ ID NO: 574, the third polypeptide chain comprises the amino acid sequence of SEQ ID NO: 576, and the fourth polypeptide chain comprises the amino acid sequence of SEQ ID NO: 573;
the first polypeptide chain comprises the amino acid sequence of SEQ ID NO: 577, the second polypeptide chain comprises the amino acid sequence of SEQ ID NO: 578, the third polypeptide chain comprises the amino acid sequence of SEQ ID NO: 579, and the fourth polypeptide chain comprises the amino acid sequence of SEQ ID NO: 577;
the first polypeptide chain comprises the amino acid sequence of SEQ ID NO: 577, the second polypeptide chain comprises the amino acid sequence of SEQ ID NO: 578, the third polypeptide chain comprises the amino acid sequence of SEQ ID NO: 580, and the fourth polypeptide chain comprises the amino acid sequence of SEQ ID NO: 577;
the first polypeptide chain comprises the amino acid sequence of SEQ ID NO: 581, the second polypeptide chain comprises the amino acid sequence of SEQ ID NO: 582, the third polypeptide chain comprises the amino acid sequence of SEQ ID NO: 583, and the fourth polypeptide chain comprises the amino acid sequence of SEQ ID NO: 581;
the first polypeptide chain comprises the amino acid sequence of SEQ ID NO: 581, the second polypeptide chain comprises the amino acid sequence of SEQ ID NO: 582, the third polypeptide chain comprises the amino acid sequence of SEQ ID NO: 584, and the fourth polypeptide chain comprises the amino acid sequence of SEQ ID NO: 581;
the first polypeptide chain comprises the amino acid sequence of SEQ ID NO: 585, the second polypeptide chain comprises the amino acid sequence of SEQ ID NO: 586, the third polypeptide chain comprises the amino acid sequence of SEQ ID NO: 587, and the fourth polypeptide chain comprises the amino acid sequence of SEQ ID NO: 585;
the first polypeptide chain comprises the amino acid sequence of SEQ ID NO: 585, the second polypeptide chain comprises the amino acid sequence of SEQ ID NO: 586, the third polypeptide chain comprises the amino acid sequence of SEQ ID NO: 588, and the fourth polypeptide chain comprises the amino acid sequence of SEQ ID NO: 585;
the first polypeptide chain comprises the amino acid sequence of SEQ ID NO: 589, the second polypeptide chain comprises the amino acid sequence of SEQ ID NO: 590, the third polypeptide chain comprises the amino acid sequence of SEQ ID NO: 591, and the fourth polypeptide chain comprises the amino acid sequence of SEQ ID NO: 589; or
the first polypeptide chain comprises the amino acid sequence of SEQ ID NO: 589, the second polypeptide chain comprises the amino acid sequence of SEQ ID NO: 590, the third polypeptide chain comprises the amino acid sequence of SEQ ID NO: 592, and the fourth polypeptide chain comprises the amino acid sequence of SEQ ID NO: 589.

Embodiment 66. The method of any one of Embodiments 1 or 4-65, wherein the IL-21 fusion protein is administered prior to the administration of the IL-2 fusion protein.

Embodiment 67. The method of any one of Embodiments 1 or 4-65, wherein the IL-2 fusion protein is administered prior to the administration of the IL-21 fusion protein.

Embodiment 68. The method of any one of Embodiments 1 or 4-65, wherein the IL-21 fusion protein and IL-2 fusion protein are administered concurrently, sequentially, or simultaneously.

Embodiment 69. The method of any one of Embodiments 1 or 4-65, wherein the IL-21 fusion protein is formulated in a pharmaceutical composition comprising a pharmaceutically acceptable carrier.

Embodiment 70. The method of any one of Embodiments 1 or 4-69, wherein the IL-2 fusion protein is formulated in a pharmaceutical composition comprising a pharmaceutically acceptable carrier.

Embodiment 71. The method of Embodiment 69 or 70, wherein the IL-21 fusion protein and the IL-2 fusion protein are formulated in a single pharmaceutical composition.

Embodiment 72. The method of Embodiment 69 or 70, wherein the IL-21 fusion protein and the IL-2 fusion protein are formulated in separate pharmaceutical compositions.

Embodiment 73. The method of any one of Embodiments 1-72, wherein the first antibody or antigen binding fragment thereof and the second antibody or antigen binding fragment thereof each target CD8ab or CD8b.

Embodiment 74. The method of Embodiment 73, wherein the first antibody or antigen binding fragment thereof and the second antibody or antigen binding fragment thereof each targets a different epitope on CD8ab or CD8b.

Embodiment 75. The method of Embodiment 73 or 74, wherein the first antibody or antigen binding fragment thereof and the second antibody or antigen binding fragment thereof do not cross compete for binding to CD8ab or CD8b.

Embodiment 76. A kit comprising one or more containers comprising (a) an IL-21 fusion protein comprising (i) a first antibody or antigen-binding fragment thereof that specifically binds to human CD8, and (ii) an IL-21 polypeptide; and (b) an IL-2 fusion protein comprising (i) a second antibody or antigen-binding fragment thereof that specifically binds human CD8, and (ii) an IL-2 polypeptide, optionally a pharmaceutically acceptable carrier, optionally instructions for administering the IL-21 fusion protein and the IL-2 fusion protein concurrently, sequentially, or simultaneously to a subject in need thereof.

Embodiment 77. A kit comprising a container comprising an IL-21 fusion protein, and instructions for administering the IL-21 fusion protein to a subject in need thereof that has received or is receiving treatment with an IL-2 fusion protein, optionally a pharmaceutically acceptable carrier, wherein the IL-21 fusion protein comprises a first antibody or antigen-binding fragment thereof that specifically binds human CD8, and an IL-21 polypeptide, and wherein the IL-2 fusion protein comprises a second antibody or antigen-binding fragment thereof that specifically binds to human CD8, and an IL-2 polypeptide.

Embodiment 78. A kit comprising a container comprising an IL-2 fusion protein, and instructions for administering the IL-2 fusion protein to a subject in need thereof that has received or is receiving treatment with an IL-21 fusion protein, optionally a pharmaceutically acceptable carrier, wherein the IL-21 fusion protein comprises a first antibody or antigen-binding fragment thereof that specifically binds human CD8, and an IL-21 polypeptide, and wherein the IL-2 fusion protein comprises a second antibody or antigen-binding fragment thereof that specifically binds to human CD8, and an IL-2 polypeptide.

Embodiment 79. The kit of any one of Embodiments 76-78, wherein the subject in need thereof has a disease, optionally wherein the disease is a cancer or an infection.

Embodiment 80. The kit of any one of Embodiments 76-79, wherein the kit induces an immune response in the subject.

Embodiment 81. The kit of Embodiment 80, wherein the immune response is a T cell response.

Embodiment 82. The kit of any of Embodiments 76-81, wherein the kit delays cancer progression in the subject.

Embodiment 83. The kit of any of Embodiments 76-82, wherein the kit reduces tumor volume in the subject.

Embodiment 84. The kit of any of Embodiments 76-83, wherein the kit reduces or inhibits tumor growth in the subject.

Embodiment 85. The kit of any of Embodiments 76-84, wherein the kit delays cancer progression, reduces tumor volume, and/or reduces or inhibits tumor growth in the subject more than treatment with either the IL-2 fusion protein or the IL-21 fusion protein alone in a subject.

Embodiment 86. A composition comprising (a) an IL-21 fusion protein comprising (i) a first antibody or antigen-binding fragment thereof that specifically binds to human CD8, and (ii) an IL-21 polypeptide; and (b) an IL-2 fusion protein comprising (i) a second antibody or antigen-binding fragment thereof that specifically binds human CD8, and (ii) an IL-2 polypeptide.

Embodiment 87. The kit of any one of Embodiments 76-85, or the composition of claim 86, wherein the first antibody or antigen-binding fragment thereof and the second antibody or antigen-binding fragment thereof are the same.

Embodiment 88. The kit or composition of any one of Embodiments 76-87, wherein the first antibody or antigen-binding fragment thereof and the second antibody or antigen-binding fragment thereof are different.

Embodiment 89. The kit or the composition of any one of Embodiments 76-88, wherein the first antibody or antigen-binding fragment thereof and the second antibody or antigen-binding fragment thereof both bind to a cell expressing a human CD8ab heterodimer on its surface with an EC50 that is less than 1000 nM.

Embodiment 90. The kit or the composition of any one of Embodiments 76-89, wherein the first antibody or antigen-binding fragment thereof and the second antibody or antigen-binding fragment thereof both bind human CD8+ T cells.

Embodiment 91. The kit or the composition of any one of Embodiments 76-90, wherein the first antibody or antigen-binding fragment thereof comprises a heavy chain and a light chain, wherein the heavy chain comprises a variable (VH) domain; and the light chain comprises a light chain variable (VL) domain, and wherein in the first antibody or antigen-binding fragment thereof:
the VH domain comprises a CDR-H1 comprising the amino acid sequence of SEQ ID NO: 13, a CDR-H2 comprising the amino acid sequence of SEQ ID NO: 14, and a CDR-H3 comprising the amino acid sequence of SEQ ID NO: 15; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 18;
the VH domain comprises a CDR-H1 comprising the amino acid sequence of SEQ ID NO: 19, a CDR-H2 comprising the amino acid sequence of SEQ ID NO: 20, and a CDR-H3 comprising the amino acid sequence of SEQ ID NO: 21; and the VL domain comprises a CDR-L1 comprising the amino acid sequence of SEQ ID NO: 22, a CDR-L2 comprising the amino acid sequence of SEQ ID NO: 23, and a CDR-L3 comprising the amino acid sequence of SEQ ID NO: 24;
the VH domain comprises a CDR-H1 comprising the amino acid sequence of SEQ ID NO: 25, a CDR-H2 comprising the amino acid sequence of SEQ ID NO: 26, and a CDR-H3 comprising the amino acid sequence of SEQ ID NO: 27; and the VL domain comprises a CDR-L1 comprising the amino acid sequence of SEQ ID NO: 28, a CDR-L2 comprising the amino acid sequence of SEQ ID NO: 29, and a CDR-L3 comprising the amino acid sequence of SEQ ID NO: 30;
the VH domain comprises a CDR-H1 comprising the amino acid sequence of SEQ ID NO: 31, a CDR-H2 comprising the amino acid sequence of SEQ ID NO: 32, and a CDR-H3 comprising the amino acid sequence of SEQ ID NO: 33; and the VL domain comprises a CDR-L1 comprising the amino acid sequence of SEQ ID NO: 34, a CDR-L2 comprising the amino acid sequence of SEQ ID NO: 35, and a CDR-L3 comprising the amino acid sequence of SEQ ID NO: 36;
the VH domain comprises a CDR-H1 comprising the amino acid sequence of SEQ ID NO: 37, a CDR-H2 comprising the amino acid sequence of SEQ ID NO: 38, and a CDR-H3 comprising the amino acid sequence of SEQ ID NO: 39; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
the VH domain comprises a CDR-H1 comprising the amino acid sequence of SEQ ID NO: 43, a CDR-H2 comprising the amino acid sequence of SEQ ID NO: 44, and a CDR-H3 comprising the amino acid sequence of SEQ ID NO: 45; and the VL domain comprises a CDR-L1 comprising the amino acid sequence of SEQ ID NO: 46, a CDR-L2 comprising the amino acid sequence of SEQ ID NO: 47, and a CDR-L3 comprising the amino acid sequence of SEQ ID NO: 48;
the VH domain comprises a CDR-H1 comprising the amino acid sequence of SEQ ID NO: 1, a CDR-H2 comprising the amino acid sequence of SEQ ID NO: 2, and a CDR-H3 comprising the amino acid sequence of SEQ ID NO: 3; and the VL domain comprises a CDR-L1 comprising the amino acid sequence of SEQ ID NO: 4, a CDR-L2 comprising the amino acid sequence of SEQ ID NO: 5, and a CDR-L3 comprising the amino acid sequence of SEQ ID NO: 6;
the VH domain comprises a CDR-H1 comprising the amino acid sequence of SEQ ID NO: 177, a CDR-H2 comprising the amino acid sequence of SEQ ID NO: 178, and a CDR-H3 comprising the amino acid sequence of SEQ ID NO: 179; and the VL domain comprises a CDR-L1 comprising the amino acid sequence of SEQ ID NO: 180, a CDR-L2 comprising the amino acid sequence of SEQ ID NO: 181, and a CDR-L3 comprising the amino acid sequence of SEQ ID NO: 182;
the VH domain comprises a CDR-H1 comprising the amino acid sequence of SEQ ID NO: 225, a CDR-H2 comprising the amino acid sequence of SEQ ID NO: 226, and a CDR-H3 comprising the amino acid sequence of SEQ ID NO: 227; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228;
the VH domain comprises a CDR-H1 comprising the amino acid sequence of SEQ ID NO: 229, a CDR-H2 comprising the amino acid sequence of SEQ ID NO: 230, and a CDR-H3 comprising the amino acid sequence of SEQ ID NO: 231; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
the VH domain comprises a CDR-H1 comprising the amino acid sequence of SEQ ID NO: 225, a CDR-H2 comprising the amino acid sequence of SEQ ID NO: 232, and a CDR-H3 comprising the amino acid sequence of SEQ ID NO: 233; and the VL domain comprises a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236;
the VH domain comprises a CDR-H1 comprising the amino acid sequence of SEQ ID NO: 225, a CDR-H2 comprising the amino acid sequence of SEQ ID NO: 232, and a CDR-H3 comprising the amino acid sequence of SEQ ID NO: 233; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228; or the VH domain comprises a CDR-H1 comprising the amino acid sequence of SEQ ID NO: 229, a CDR-H2 comprising the amino acid sequence of SEQ ID NO: 237, and a CDR-H3 comprising the amino acid sequence of SEQ ID NO: 231; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42.

Embodiment 92. The method of any one of Embodiments 76-91, wherein the first antibody or antigen-binding fragment thereof comprises a heavy chain and a light chain, wherein the heavy chain comprises a heavy chain variable (VH) domain, and the light chain comprises a light chain variable (VL) domain, and wherein in the first antibody or antigen-binding fragment thereof:
the VH domain comprises a CDR-H1 comprising the amino acid sequence of SEQ ID NO: 51, a CDR-H2 comprising the amino acid sequence of SEQ ID NO: 52, and a CDR-H3 comprising the amino acid sequence of SEQ ID NO: 15; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 18;
the VH domain comprises a CDR-H1 comprising the amino acid sequence of SEQ ID NO: 53, a CDR-H2 comprising the amino acid sequence of SEQ ID NO: 52, and a CDR-H3 comprising the amino acid sequence of SEQ ID NO: 21; and the VL domain comprises a CDR-L1 comprising the amino acid sequence of SEQ ID NO: 22, a CDR-L2 comprising the amino acid sequence of SEQ ID NO: 23, and a CDR-L3 comprising the amino acid sequence of SEQ ID NO: 24;
the VH domain comprises a CDR-H1 comprising the amino acid sequence of SEQ ID NO: 49, a CDR-H2 comprising the amino acid sequence of SEQ ID NO: 52, and a CDR-H3 comprising the amino acid sequence of SEQ ID NO: 27; and the VL domain comprises a CDR-L1 comprising the amino acid sequence of SEQ ID NO: 28, a CDR-L2 comprising the amino acid sequence of SEQ ID NO: 29, and a CDR-L3 comprising the amino acid sequence of SEQ ID NO: 30;
the VH domain comprises a CDR-H1 comprising the amino acid sequence of SEQ ID NO: 54, a CDR-H2 comprising the amino acid sequence of SEQ ID NO: 52, and a CDR-H3 comprising the amino acid sequence of SEQ ID NO: 33; and the VL domain comprises a CDR-L1 comprising the amino acid sequence of SEQ ID NO: 34, a CDR-L2 comprising the amino acid sequence of SEQ ID NO: 35, and a CDR-L3 comprising the amino acid sequence of SEQ ID NO: 36;
the VH domain comprises a CDR-H1 comprising the amino acid sequence of SEQ ID NO: 55, a CDR-H2 comprising the amino acid sequence of SEQ ID NO: 56, and a CDR-H3 comprising the amino acid sequence of SEQ ID NO: 39; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
the VH domain comprises a CDR-H1 comprising the amino acid sequence of SEQ ID NO: 55, a CDR-H2 comprising the amino acid sequence of SEQ ID NO: 57, and a CDR-H3 comprising the amino acid sequence of SEQ ID NO: 45; and the VL domain comprises a CDR-L1 comprising the amino acid sequence of SEQ ID NO: 46, a CDR-L2 comprising the amino acid sequence of SEQ ID NO: 47, and a CDR-L3 comprising the amino acid sequence of SEQ ID NO: 48;
the VH domain comprises a CDR-H1 comprising the amino acid sequence of SEQ ID NO: 49, a CDR-H2 comprising the amino acid sequence of SEQ ID NO: 50, and a CDR-H3 comprising the amino acid sequence of SEQ ID NO: 3; and the VL domain comprises a CDR-L1 comprising the amino acid sequence of SEQ ID NO: 4, a CDR-L2 comprising the amino acid sequence of SEQ ID NO: 5, and a CDR-L3 comprising the amino acid sequence of SEQ ID NO: 6;
the VH domain comprises a CDR-H1 comprising the amino acid sequence of SEQ ID NO: 183, a CDR-H2 comprising the amino acid sequence of SEQ ID NO: 184, and a CDR-H3 comprising the amino acid sequence of SEQ ID NO: 179; and the VL domain comprises a CDR-L1 comprising the amino acid sequence of SEQ ID NO: 180, a CDR-L2 comprising the amino acid sequence of SEQ ID NO: 181, and a CDR-L3 comprising the amino acid sequence of SEQ ID NO: 182;
the VH domain comprises a CDR-H1 comprising the amino acid sequence of SEQ ID NO: 238, a CDR-H2 comprising the amino acid sequence of SEQ ID NO: 239, and a CDR-H3 comprising the amino acid sequence of SEQ ID NO: 233; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228;
the VH domain comprises a CDR-H1 comprising the amino acid sequence of SEQ ID NO: 240, a CDR-H2 comprising the amino acid sequence of SEQ ID NO: 241, and a CDR-H3 comprising the amino acid sequence of SEQ ID NO: 242; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
the VH domain comprises a CDR-H1 comprising the amino acid sequence of SEQ ID NO: 238, a CDR-H2 comprising the amino acid sequence of SEQ ID NO: 243, and a CDR-H3 comprising the amino acid sequence of SEQ ID NO: 233; and the VL domain comprises a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236;
the VH domain comprises a CDR-H1 comprising the amino acid sequence of SEQ ID NO: 238, a CDR-H2 comprising the amino acid sequence of SEQ ID NO: 243, and a CDR-H3 comprising the amino acid sequence of SEQ ID NO: 233; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228; or the VH domain comprises a CDR-H1 comprising the amino acid sequence of SEQ ID NO: 240, a CDR-H2 comprising the amino acid sequence of SEQ ID NO: 244, and a CDR-H3 comprising the amino acid sequence of SEQ ID NO: 242; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42.

Embodiment 93. The kit of Embodiment 91 or 92, wherein the VH and VL domains comprise amino acid sequences of:
(a) SEQ ID NOs: 58 and 59, respectively;
(b) SEQ ID NOs: 247 and 248, respectively;
(c) SEQ ID NOs: 62 and 63, respectively;
(d) SEQ ID NOs: 64 and 65, respectively;
(e) SEQ ID NOs: 66 and 67, respectively;
(f) SEQ ID NOs: 68 and 69, respectively;
(g) SEQ ID NOs: 70 and 71, respectively;
(h) SEQ ID NOs: 72 and 73, respectively;
(i) SEQ ID NOs: 185 and 186, respectively;
(j) SEQ ID NOs: 245 and 246, respectively;
(k) SEQ ID NOs: 249 and 250, respectively;
(l) SEQ ID NOs: 251 and 252, respectively;
(m) SEQ ID NOs: 253 and 254, respectively;
(n) SEQ ID NOs: 255 and 256, respectively; or
(o) SEQ ID NOs: 257 and 258, respectively.

Embodiment 94. The kit or the composition of any one of Embodiments 76-93, wherein the second antibody or antigen-binding fragment thereof comprises a heavy chain and a light chain, wherein the heavy chain comprises a variable (VH) domain; and the light chain comprises a light chain variable (VL) domain, and wherein in the second antibody or antigen-binding fragment thereof:
the VH domain comprises a CDR-H1 comprising the amino acid sequence of SEQ ID NO: 13, a CDR-H2 comprising the amino acid sequence of SEQ ID NO: 14, and a CDR-H3 comprising the amino acid sequence of SEQ ID NO: 15; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 18;
the VH domain comprises a CDR-H1 comprising the amino acid sequence of SEQ ID NO: 19, a CDR-H2 comprising the amino acid sequence of SEQ ID NO: 20, and a CDR-H3 comprising the amino acid sequence of SEQ ID NO: 21; and the VL domain comprises a CDR-L1 comprising the amino acid sequence of SEQ ID NO: 22, a CDR-L2 comprising the amino acid sequence of SEQ ID NO: 23, and a CDR-L3 comprising the amino acid sequence of SEQ ID NO: 24;
the VH domain comprises a CDR-H1 comprising the amino acid sequence of SEQ ID NO: 25, a CDR-H2 comprising the amino acid sequence of SEQ ID NO: 26, and a CDR-H3 comprising the amino acid sequence of SEQ ID NO: 27; and the VL domain comprises a CDR-L1 comprising the amino acid sequence of SEQ ID NO: 28, a CDR-L2 comprising the amino acid sequence of SEQ ID NO: 29, and a CDR-L3 comprising the amino acid sequence of SEQ ID NO: 30;
the VH domain comprises a CDR-H1 comprising the amino acid sequence of SEQ ID NO: 31, a CDR-H2 comprising the amino acid sequence of SEQ ID NO: 32, and a CDR-H3 comprising the amino acid sequence of SEQ ID NO: 33; and the VL domain comprises a CDR-L1 comprising the amino acid sequence of SEQ ID NO: 34, a CDR-L2 comprising the amino acid sequence of SEQ ID NO: 35, and a CDR-L3 comprising the amino acid sequence of SEQ ID NO: 36;
the VH domain comprises a CDR-H1 comprising the amino acid sequence of SEQ ID NO: 37, a CDR-H2 comprising the amino acid sequence of SEQ ID NO: 38, and a CDR-H3 comprising the amino acid sequence of SEQ ID NO: 39; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
the VH domain comprises a CDR-H1 comprising the amino acid sequence of SEQ ID NO: 43, a CDR-H2 comprising the amino acid sequence of SEQ ID NO: 44, and a CDR-H3 comprising the amino acid sequence of SEQ ID NO: 45; and the VL domain comprises a CDR-L1 comprising the amino acid sequence of SEQ ID NO: 46, a CDR-L2 comprising the amino acid sequence of SEQ ID NO: 47, and a CDR-L3 comprising the amino acid sequence of SEQ ID NO: 48;
the VH domain comprises a CDR-H1 comprising the amino acid sequence of SEQ ID NO: 1, a CDR-H2 comprising the amino acid sequence of SEQ ID NO: 2, and a CDR-H3 comprising the amino acid sequence of SEQ ID NO: 3; and the VL domain comprises a CDR-L1 comprising the amino acid sequence of SEQ ID NO: 4, a CDR-L2 comprising the amino acid sequence of SEQ ID NO: 5, and a CDR-L3 comprising the amino acid sequence of SEQ ID NO: 6;
the VH domain comprises a CDR-H1 comprising the amino acid sequence of SEQ ID NO: 177, a CDR-H2 comprising the amino acid sequence of SEQ ID NO: 178, and a CDR-H3 comprising the amino acid sequence of SEQ ID NO: 179; and the VL domain comprises a CDR-L1 comprising the amino acid sequence of SEQ ID NO: 180, a CDR-L2 comprising the amino acid sequence of SEQ ID NO: 181, and a CDR-L3 comprising the amino acid sequence of SEQ ID NO: 182;
the VH domain comprises a CDR-H1 comprising the amino acid sequence of SEQ ID NO: 225, a CDR-H2 comprising the amino acid sequence of SEQ ID NO: 226, and a CDR-H3 comprising the amino acid sequence of SEQ ID NO: 227; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228;
the VH domain comprises a CDR-H1 comprising the amino acid sequence of SEQ ID NO: 229, a CDR-H2 comprising the amino acid sequence of SEQ ID NO: 230, and a CDR-H3 comprising the amino acid sequence of SEQ ID NO: 231; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
the VH domain comprises a CDR-H1 comprising the amino acid sequence of SEQ ID NO: 225, a CDR-H2 comprising the amino acid sequence of SEQ ID NO: 232, and a CDR-H3 comprising the amino acid sequence of SEQ ID NO: 233; and the VL domain comprises a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236;
the VH domain comprises a CDR-H1 comprising the amino acid sequence of SEQ ID NO: 225, a CDR-H2 comprising the amino acid sequence of SEQ ID NO: 232, and a CDR-H3 comprising the amino acid sequence of SEQ ID NO: 233; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228; or
the VH domain comprises a CDR-H1 comprising the amino acid sequence of SEQ ID NO: 229, a CDR-H2 comprising the amino acid sequence of SEQ ID NO: 237, and a CDR-H3 comprising the amino acid sequence of SEQ ID NO: 231; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42.
   the antibody or antigen-binding fragment thereof of each of the IL-21 and IL-2 fusion proteins comprises a heavy chain variable (VH) domain and a light chain variable (VL) domain, and wherein:
the VH domain comprises a CDR-H1 comprising the amino acid sequence of SEQ ID NO: 238, a CDR-H2 comprising the amino acid sequence of SEQ ID NO: 243, and a CDR-H3 comprising the amino acid sequence of SEQ ID NO: 233; and wherein the VL domain comprises a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236; or
the VH domain comprises a CDR-H1 comprising the amino acid sequence of SEQ ID NO: 225, a CDR-H2 comprising the amino acid sequence of SEQ ID NO: 232, and a CDR-H3 comprising the amino acid sequence of SEQ ID NO: 233; and wherein the VL domain comprises a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236.

Embodiment 95. The kit or the composition of any one of Embodiments 76-94, wherein the second antibody or antigen-binding fragment thereof comprises a heavy chain and a light chain, wherein the heavy chain comprises a heavy chain variable (VH) domain, and the light chain comprises a light chain variable (VL) domain, and wherein in the second antibody or antigen-binding fragment thereof:
the VH domain comprises a CDR-H1 comprising the amino acid sequence of SEQ ID NO: 51, a CDR-H2 comprising the amino acid sequence of SEQ ID NO: 52, and a CDR-H3 comprising the amino acid sequence of SEQ ID NO: 15; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 18;
the VH domain comprises a CDR-H1 comprising the amino acid sequence of SEQ ID NO: 53, a CDR-H2 comprising the amino acid sequence of SEQ ID NO: 52, and a CDR-H3 comprising the amino acid sequence of SEQ ID NO: 21; and the VL domain comprises a CDR-L1 comprising the amino acid sequence of SEQ ID NO: 22, a CDR-L2 comprising the amino acid sequence of SEQ ID NO: 23, and a CDR-L3 comprising the amino acid sequence of SEQ ID NO: 24;
the VH domain comprises a CDR-H1 comprising the amino acid sequence of SEQ ID NO: 49, a CDR-H2 comprising the amino acid sequence of SEQ ID NO: 52, and a CDR-H3 comprising the amino acid sequence of SEQ ID NO: 27; and the VL domain comprises a CDR-L1 comprising the amino acid sequence of SEQ ID NO: 28, a CDR-L2 comprising the amino acid sequence of SEQ ID NO: 29, and a CDR-L3 comprising the amino acid sequence of SEQ ID NO: 30;
the VH domain comprises a CDR-H1 comprising the amino acid sequence of SEQ ID NO: 54, a CDR-H2 comprising the amino acid sequence of SEQ ID NO: 52, and a CDR-H3 comprising the amino acid sequence of SEQ ID NO: 33; and the VL domain comprises a CDR-L1 comprising the amino acid sequence of SEQ ID NO: 34, a CDR-L2 comprising the amino acid sequence of SEQ ID NO: 35, and a CDR-L3 comprising the amino acid sequence of SEQ ID NO: 36;
the VH domain comprises a CDR-H1 comprising the amino acid sequence of SEQ ID NO: 55, a CDR-H2 comprising the amino acid sequence of SEQ ID NO: 56, and a CDR-H3 comprising the amino acid sequence of SEQ ID NO: 39; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
the VH domain comprises a CDR-H1 comprising the amino acid sequence of SEQ ID NO: 55, a CDR-H2 comprising the amino acid sequence of SEQ ID NO: 57, and a CDR-H3 comprising the amino acid sequence of SEQ ID NO: 45; and the VL domain comprises a CDR-L1 comprising the amino acid sequence of SEQ ID NO: 46, a CDR-L2 comprising the amino acid sequence of SEQ ID NO: 47, and a CDR-L3 comprising the amino acid sequence of SEQ ID NO: 48;
the VH domain comprises a CDR-H1 comprising the amino acid sequence of SEQ ID NO: 49, a CDR-H2 comprising the amino acid sequence of SEQ ID NO: 50, and a CDR-H3 comprising the amino acid sequence of SEQ ID NO: 3; and the VL domain comprises a CDR-L1 comprising the amino acid sequence of SEQ ID NO: 4, a CDR-L2 comprising the amino acid sequence of SEQ ID NO: 5, and a CDR-L3 comprising the amino acid sequence of SEQ ID NO: 6;
the VH domain comprises a CDR-H1 comprising the amino acid sequence of SEQ ID NO: 183, a CDR-H2 comprising the amino acid sequence of SEQ ID NO: 184, and a CDR-H3 comprising the amino acid sequence of SEQ ID NO: 179; and the VL domain comprises a CDR-L1 comprising the amino acid sequence of SEQ ID NO: 180, a CDR-L2 comprising the amino acid sequence of SEQ ID NO: 181, and a CDR-L3 comprising the amino acid sequence of SEQ ID NO: 182;
the VH domain comprises a CDR-H1 comprising the amino acid sequence of SEQ ID NO: 238, a CDR-H2 comprising the amino acid sequence of SEQ ID NO: 239, and a CDR-H3 comprising the amino acid sequence of SEQ ID NO: 233; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228;
the VH domain comprises a CDR-H1 comprising the amino acid sequence of SEQ ID NO: 240, a CDR-H2 comprising the amino acid sequence of SEQ ID NO: 241, and a CDR-H3 comprising the amino acid sequence of SEQ ID NO: 242; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
the VH domain comprises a CDR-H1 comprising the amino acid sequence of SEQ ID NO: 238, a CDR-H2 comprising the amino acid sequence of SEQ ID NO: 243, and a CDR-H3 comprising the amino acid sequence of SEQ ID NO: 233; and the VL domain comprises a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236;
the VH domain comprises a CDR-H1 comprising the amino acid sequence of SEQ ID NO: 238, a CDR-H2 comprising the amino acid sequence of SEQ ID NO: 243, and a CDR-H3 comprising the amino acid sequence of SEQ ID NO: 233; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228; or
the VH domain comprises a CDR-H1 comprising the amino acid sequence of SEQ ID NO: 240, a CDR-H2 comprising the amino acid sequence of SEQ ID NO: 244, and a CDR-H3 comprising the amino acid sequence of SEQ ID NO: 242; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42.

Embodiment 96. The kit or the composition of any one of Embodiment 94 or 95, wherein the VH and VL domains of the second antibody or antigen-binding fragment thereof comprise the amino acid sequences of:
(a) SEQ ID NOs: 58 and 59, respectively;
(b) SEQ ID NOs: 247 and 248, respectively;
(c) SEQ ID NOs: 62 and 63, respectively;
(d) SEQ ID NOs: 64 and 65, respectively;
(e) SEQ ID NOs: 66 and 67, respectively;
(f) SEQ ID NOs: 68 and 69, respectively;
(g) SEQ ID NOs: 70 and 71, respectively;
(h) SEQ ID NOs: 72 and 73, respectively;
(i) SEQ ID NOs: 185 and 186, respectively;
(j) SEQ ID NOs: 245 and 246, respectively;
(k) SEQ ID NOs: 249 and 250, respectively;
(l) SEQ ID NOs: 251 and 252, respectively;
(m) SEQ ID NOs: 253 and 254, respectively;
(n) SEQ ID NOs: 255 and 256, respectively; or
(o) SEQ ID NOs: 257 and 258, respectively.

Embodiment 97. The kit or the composition of any one of Embodiments 76-96, wherein (i) the IL-21 polypeptide is linked to the first antibody or antigen binding fragment directly or by a linker, and/or (ii) the IL-2 polypeptide is linked to the second antibody or antigen binding fragment directly or by a linker.

Embodiment 98. The kit of Embodiment 97, wherein (i) the IL-21 polypeptide is linked to the first antibody or antigen binding fragment by a Gly-Ser linker, and/or (ii) the IL-2 polypeptide is linked to the second antibody or antigen binding fragment by a Gly-Ser linker.

Embodiment 99. The kit or the composition of any one of Embodiments 76-98, wherein the IL-2 fusion protein, the IL-21 fusion protein, or both the IL-2 and IL-21 fusion proteins each comprise:
two antibody heavy chain polypeptides comprising a structure according to formula [I], from N-terminus to C-terminus:

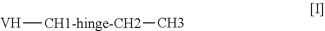

and two antibody light chain polypeptides comprising a structure according to formula [II], from N-terminus to C-terminus:

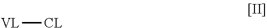

wherein VH is the VH domain, wherein CH1 is an antibody CH1 domain, wherein hinge is an antibody hinge domain, wherein CH2-CH3 is an antibody Fc domain, wherein VL is the VL domain, and wherein CL is an antibody constant light chain domain; and
wherein the N-terminus of the IL-2 polypeptide or the IL-21 polypeptide is fused to the C-terminus of one of the two CH3 domains.

Embodiment 100. The kit of Embodiment 99, wherein one or both of the two antibody heavy chain polypeptides comprise(s) the following amino acid substitutions: L234A, L235A, and G237A, numbering according to EU index; optionally wherein a first of the two antibody heavy chain polypeptides comprises amino acid substitutions Y349C and T366W, and a second of the two antibody heavy chain polypeptides comprises amino acid substitutions S354C, T366S, L368A and Y407V, numbering according to EU index.

Embodiment 101. The kit or the composition of any one of Embodiments 76-100, wherein the IL-2 polypeptide has a binding affinity to IL-2Rα that is reduced by 50% or more, compared to binding affinity of a human IL-2 polypeptide comprising SEQ ID NO: 81.

Embodiment 102. The kit of Embodiment 101, wherein the IL-2 polypeptide comprises one or more amino acid mutations relative to a human IL-2 polypeptide comprising SEQ ID NO: 81 that reduces the binding affinity of the IL-2 polypeptide.

Embodiment 103. The kit or the composition of any one of Embodiments 76-100, wherein the IL-2 polypeptide comprises the amino acid sequence of SEQ ID NO: 81 with one, two, three, four, or five amino acid substitutions relative to SEQ ID NO: 81, and wherein the one, two, three, four, or five substitution(s) comprise substitution(s) at positions of SEQ ID NO: 81 selected from the group consisting of: Q11, H16, L18, L19, D20, Q22, R38, F42, K43, Y45, E62, P65, E68, V69, L72, D84, S87, N88, V91, 192, T123, Q126, S127, 1129, and S130.

Embodiment 104. The kit of Embodiment 103, wherein the IL-2 polypeptide comprises the sequence of SEQ ID NO: 81 with a set of amino acid substitutions relative to the sequence of SEQ ID NO: 81 selected from the group consisting of: R38E and F42A; R38D and F42A; F42A and E62Q; R38A and F42K; R38E, F42A, and N88S; R38E, F42A, and N88A; R38E, F42A, and N88G; R38E, F42A, and N88R; R38E, F42A, and N88T; R38E, F42A, and N88D; R38E, F42A, and V91E; R38E, F42A, and D84H; R38E, F42A, and D84K; R38E, F42A, and D84R; H16D, R38E and F42A; H16E, R38E and F42A; R38E, F42A and Q126S; R38D, F42A and N88S; R38D, F42A and N88A; R38D, F42A and N88G; R38D, F42A and N88R; R38D, F42A and N88T; R38D, F42A and N88D; R38D, F42A and V91E; R38D, F42A, and D84H; R38D, F42A, and D84K; R38D, F42A, and D84R; H16D, R38D and F42A; H16E, R38D and F42A; R38D, F42A and Q126S; R38A, F42K, and N88S; R38A, F42K, and N88A; R38A, F42K, and N88G; R38A, F42K, and N88R; R38A, F42K, and N88T; R38A, F42K, and N88D; R38A, F42K, and V91E; R38A, F42K, and D84H; R38A, F42K, and D84K; R38A, F42K, and D84R; H16D, R38A, and F42K; H16E, R38A, and F42K; R38A, F42K, and Q126S; F42A, E62Q, and N88S; F42A, E62Q, and N88A; F42A, E62Q, and N88G; F42A, E62Q, and N88R; F42A, E62Q, and N88T; F42A, E62Q, and N88D; F42A, E62Q, and V91E; F42A, E62Q, and D84H; F42A, E62Q, and D84K; and F42A, E62Q, and D84R.

Embodiment 105. The kit of Embodiment 103, wherein the IL-2 polypeptide comprises the sequence of SEQ ID NO: 81 with a further amino acid substitution relative to SEQ ID NO: 81 at position C125.

Embodiment 106. The kit of Embodiment 105, wherein the IL-2 polypeptide comprises the sequence of SEQ ID NO: 81 with a set of amino acid substitutions relative to the sequence of SEQ ID NO: 81 selected from the group consisting of: R38E, F42A, and C125A; R38D, F42A, and C125A; F42A, E62Q, and C125A; R38A, F42K, and C125A; R38E, F42A, N88S, and C125A; R38E, F42A, N88A, and C125A; R38E, F42A, N88G, and C125A; R38E, F42A, N88R, and C125A; R38E, F42A, N88D, and C125A; R38E, F42A, N88T, and C125A; R38E, F42A, V91E, and C125A; R38E, F42A, D84H, and C125A; R38E, F42A, D84K, and C125A; R38E, F42A, D84R, and C125A; H16D, R38E, F42A, and C125A; H16E, R38E, F42A, and C125A; R38E, F42A, C125A and Q126S; R38D, F42A, N88S, and C125A; R38D, F42A, N88A, and C125A; R38D, F42A, N88G, and C125A; R38D, F42A, N88R, and C125A; R38D, F42A, N88T, and C125A; R38D, F42A, N88D, and C125A; R38D, F42A, V91E, and C125A; R38D, F42A, D84H, and C125A; R38D, F42A, D84K, and C125A; R38D, F42A, D84R, and C125A; H16D, R38D, F42A, and C125A; H16E, R38D, F42A, and C125A; R38D, F42A, C125A, and Q126S; R38A, F42K, N88S, and C125A; R38A, F42K, N88G, and C125A; R38A, F42K, N88R, and C125A; R38A, F42K, N88T, and C125A; R38A, F42K, N88D, and C125A; R38A, F42K, N88A, and C125A; R38A, F42K, V91E, and C125A; R38A, F42K, D84H, and C125A; R38A, F42K, D84K, and C125A; R38A, F42K, D84R, and C125A; H16D, R38A, F42K, and C125A; H16E, R38A, F42K, and C125A; R38A, F42K, C125A and Q126S; F42A, E62Q, N88S, and C125A; F42A, E62Q, N88A, and C125A; F42A, E62Q, N88G, and C125A; F42A, E62Q, N88R, and C125A; F42A, E62Q, N88T, and C125A; F42A, E62Q, N88D, and C125A; F42A, E62Q, V91E, and C125A; F42A, E62Q, and D84H, and C125A; F42A, E62Q, and D84K, and C125A; F42A, E62Q, and D84R, and C125A; H16D, F42A, and E62Q, and C125A; H16E, F42A, E62Q, and C125A; F42A, E62Q, C125A and Q126S; F42A, N88S, and C125A; F42A, N88A, and C125A; F42A, N88G, and C125A; F42A, N88R, and C125A; F42A, N88T, and C125A; F42A, N88D, and C125A; F42A, V91E, and C125A; F42A, D84H, and C125A; F42A, D84K, and C125A; F42A, D84R, and C125A; H16D, F42A, and C125A; H16E, F42A, and C125A; and F42A, C125A and Q126S.

Embodiment 107. The kit or the composition of any one of Embodiments 76-106, wherein the IL-2 polypeptide comprises the amino acid sequence of SEQ ID NO: 80 or SEQ ID NO: 297.

Embodiment 108. The kit or the composition of any one of Embodiments 76-106, wherein the IL-2 polypeptide comprises a sequence selected from the group consisting of SEQ ID NOs: 80, 85-155, 190-216, 297, and 354-383.

Embodiment 109. The kit or the composition of any one of Embodiments 76-108, wherein the IL-21 polypeptide comprises an amino acid sequence that is at least 80% identical to a human IL-21 polypeptide comprising SEQ ID NO: 390.

Embodiment 110. The kit of Embodiment 109, wherein the IL-21 polypeptide has an isoelectric point that is at least about 0.6 units to about 5 units lower, compared to that of the human IL-21 polypeptide.

Embodiment 111. The kit of Embodiment 110, wherein the IL-21 polypeptide comprises at least one amino acid substitution that reduces the isoelectric point of the IL-21 polypeptide by about 0.6 units to about 5 units relative to the human IL-21 polypeptide without the amino acid substitution.

Embodiment 112. The kit of Embodiment 110, wherein the IL-21 polypeptide comprises at least four amino acid substitutions that reduce the isoelectric point of the IL-21 polypeptide by about 0.6 units to about 5 units relative to the human IL-21 polypeptide without the amino acid substitutions.

Embodiment 113. The kit of Embodiment 111 or 112, wherein the IL-21 polypeptide comprises up to 10, 11, 12, 13, 14, 15, 16, 17, 18, 19 or 20 amino acid substitutions that reduce the isoelectric point.

Embodiment 114. The kit or the composition of any one of Embodiments 109-113, wherein the human IL-21 polypeptide has an isoelectric point of about 9.42.

Embodiment 115. The kit or the composition of any one of Embodiments 109-114, wherein the IL-21 polypeptide has an isoelectric point of about 7.12 to about 8.72.

Embodiment 116. The kit or the composition of any one of Embodiments 109-115, wherein the IL-21 polypeptide results in an improved exposure following administration to the subject relative to the human IL-21 polypeptide, as measured by at least about 1.5 times greater area under the curve (AUC).

Embodiment 117. The kit or the composition of any one of Embodiments 110-114, wherein the IL-21 polypeptide comprises a modified region, wherein relative to a region of the human IL-21 polypeptide that comprises about 2 to 20 positively charged amino acid residues, the modified region comprises one or more amino acid substitutions of the about 2 to 20 positively charged amino acid residues.

Embodiment 118. The kit of Embodiment 117, wherein the modified region excludes amino acid residues that bind a human IL-21 receptor.

Embodiment 119. The kit of Embodiment 117 or 118, wherein the modified region comprises amino acid residues positioned at S80 to T92 of the human IL-21 polypeptide comprising SEQ ID NO. 390.

Embodiment 120. The kit or the composition of any one of Embodiments 117-119, wherein the human IL-21 polypeptide comprises at least one positively charged amino acid residue on the surface of the human IL-21 polypeptide in a three-dimensional structure of the human IL-21 polypeptide and does not bind an IL-21 receptor, and wherein the IL-21 polypeptide comprises at least one amino acid substitution of at the least one positively charged amino acid residue.

Embodiment 121. The kit or the composition of any one of Embodiments 117-120, wherein the IL-21 polypeptide does not comprise an amino acid substitution at G84 of the human IL-21 polypeptide comprising SEQ ID NO: 390.

Embodiment 122. The kit or the composition of any one of Embodiments 110-121, wherein the IL-21 polypeptide comprises 4, 5, 6, 7, 8, 9, 10, 11, or 12 amino acid substitutions at positions relative to SEQ ID NO: 81 selected from the group consisting of: S80, T81, N82, A83, R85, R86, Q87, K88, H89, R90, L91, and T92 of SEQ ID NO: 390.

Embodiment 123. The kit of Embodiment 122, wherein the IL-21 polypeptide comprises 4, 5, 6, 7, 8, 9, 10, 11, or 12 amino acid substitutions selected from the group consisting of: S80G, T81G, N82G, N82E, A83G, A83E, A83S, R85G, R85E, R85S, R86G, R86E, Q87G, Q87E, Q87S, K88G, H89G, H89S, R90G, R90S, R90E, R90A, L91G, L91S, T92G, and T92S.

Embodiment 124. The kit of Embodiment 122, wherein the IL-21 polypeptide comprises (a) R85G, R86G, K88G and R90E, or (b) S80G, T81G, N82E, A83G, R85G, R86G, Q87G, K88G, H89G, R90E, L91G, and T92G.

Embodiment 125. The kit or the composition of any one of Embodiments 76-124, wherein the IL-21 polypeptide comprises the amino acid sequence: QGQDRHMIRMRQLIDIVDQLKNYVNDLVPEFLPAPEDVETNCEWSAFSCFQKAQLK SANTGNNERIINVSIKKLKRKPPX₁X₂X₃X₄GX₅X₆X₇X₈X₉X₁₀X₁₁X₁₂CPSCDSYEKKPPK EFLERFKSLLQKMIHQHLSSRTHGSEDS (SEQ ID NO: 554), wherein X₁=G, S; X₂=G, T; X₃=G, E, N; X₄=G, S, E, A; X₅=G, E, S, R; X₆=G, E, R; X₇=S, G, E, Q; X₈=G, K; X₉=G, S, H; X₁₀=A, E, S, G, R; X₁₁=S, G, L; and X₁₂=G, S, T, provided at least one of X₅, X₆, X₈, X₉, and X₁₀ is not the amino acid residue at the identical position set forth in SEQ ID NO: 390 optionally wherein:

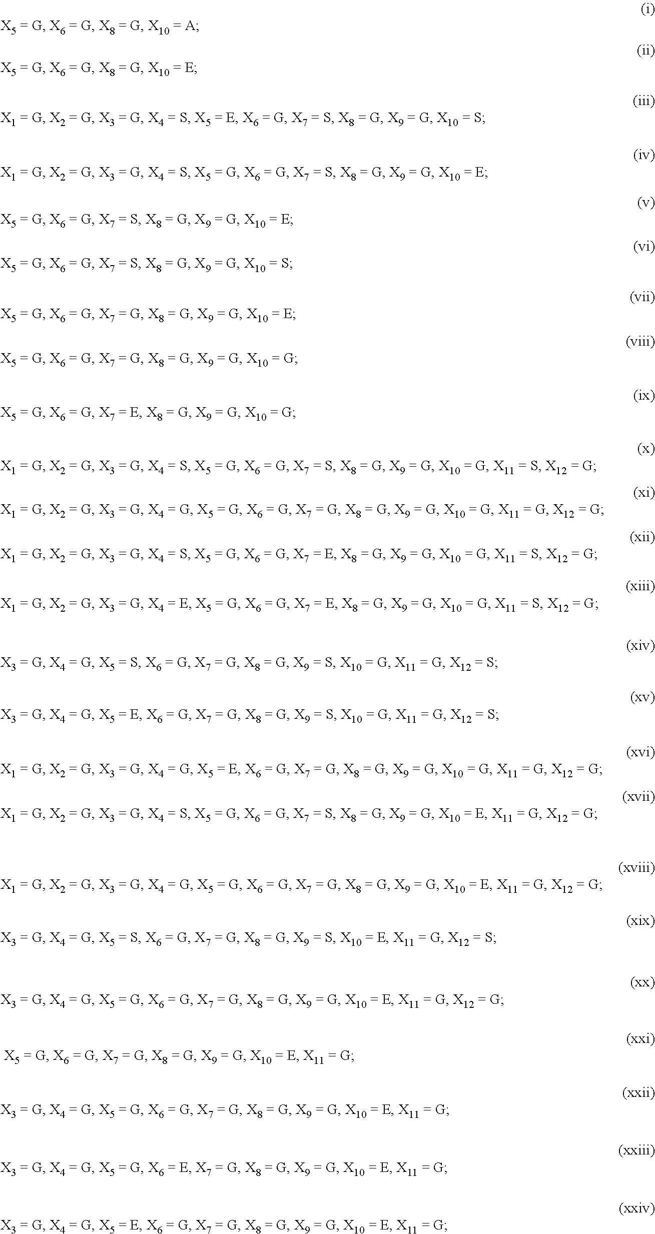

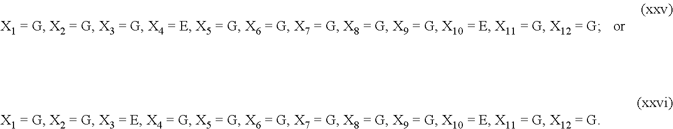

Embodiment 126. The kit or the composition of any one of Embodiments 76-125, wherein the IL-12 polypeptide comprises an amino acid sequence at least 90% identical to an amino acid sequence selected from SEQ ID NOs: 391-421.

Embodiment 127. The kit of Embodiment 126, wherein the IL-21 polypeptide comprises an amino acid sequence selected from SEQ ID NOs: 391-421.

Embodiment 128. The fusion protein of Embodiment 127, wherein the IL-21 polypeptide comprises the amino acid sequence of SEQ ID NO: 421.

Embodiment 129. The kit or the composition of any one of Embodiments 76-128, wherein the IL-21 polypeptide comprises at least one amino acid substitution that reduces binding to an IL-21 receptor compared to binding by the human IL-21 polypeptide.

Embodiment 130. The kit of Embodiment 129, wherein the at least one amino acid substitution is at one or more amino acid residues at positions R5, I8, R9, R11, L13, I14, I16, V17, D18, K72, K73, L74, K75, R76, K77, or K117, of SEQ ID NO: 390.

Embodiment 131. The kit of Embodiment 130, wherein the at least one amino acid substitution is selected from: R5F, R5A, R5E, R5S, R5T, R5N, R5Q, R5V, R5I, R5L, R5Y, 18E, R9A, R9D, R9E, R9H, R9S, R9T, R9N, R9G, R9V, R9I, R9L, R9Y, RI1D, RI1E, L13F, L13R, 114D, 116A, 116S, 116R, V171, V17A, D18A, K72A, K72E, K73A, K73E, K75A, K75E, L74I, L74F, L74M, L74V, R76E, R76F, R76A, R76N, R76D, R76S, R76T, R76Q, R76V, R76I, R76L, R76Y, R76M, K77A, K77E, and K117A.

Embodiment 132. The kit of Embodiment 130 or 131, wherein the at least one amino acid substitution is R76E or R76Q.

Embodiment 133. The kit or the composition of any one of Embodiments 76-132, wherein the IL-21 polypeptide comprises an amino acid sequence at least 90% identical to an amino acid sequence selected from SEQ ID NOs: 422-491.

Embodiment 134. The kit of Embodiment 133, wherein the IL-21 polypeptide comprises an amino acid sequence selected from SEQ ID NOs: 422-491.

Embodiment 135. The kit or the composition of any one of Embodiments 76-134, wherein the IL-2 fusion protein comprises:
one or two light chains comprising the amino acid sequence of SEQ ID NO: 156, a heavy chain comprising the amino acid sequence of SEQ ID NO: 157, and a heavy chain comprising the amino acid sequence of SEQ ID NO: 158;
one or two light chains comprising the amino acid sequence of SEQ ID NO: 159, a heavy chain comprising the amino acid sequence of SEQ ID NO: 160, and a heavy chain comprising the amino acid sequence of SEQ ID NO: 161;
one or two light chains comprising the amino acid sequence of SEQ ID NO: 162, a heavy chain comprising the amino acid sequence of SEQ ID NO: 163, and a heavy chain comprising the amino acid sequence of SEQ ID NO: 164;
one or two light chains comprising the amino acid sequence of SEQ ID NO: 165, a heavy chain comprising the amino acid sequence of SEQ ID NO: 166, and a heavy chain comprising the amino acid sequence of SEQ ID NO: 167;
one or two light chains comprising the amino acid sequence of SEQ ID NO: 168, a heavy chain comprising the amino acid sequence of SEQ ID NO: 169, and a heavy chain comprising the amino acid sequence of SEQ ID NO: 170;
one or two light chains comprising the amino acid sequence of SEQ ID NO: 171, a heavy chain comprising the amino acid sequence of SEQ ID NO: 172, and a heavy chain comprising the amino acid sequence of SEQ ID NO: 173;
one or two light chains comprising the amino acid sequence of SEQ ID NO: 174, a heavy chain comprising the amino acid sequence of SEQ ID NO: 175, and a heavy chain comprising the amino acid sequence of SEQ ID NO: 176;
one or two light chains comprising the amino acid sequence of SEQ ID NO: 187, a heavy chain comprising the amino acid sequence of SEQ ID NO: 188, and a heavy chain comprising the amino acid sequence of SEQ ID NO: 189;
one or two light chains comprising the amino acid sequence of SEQ ID NO: 298, a heavy chain comprising the amino acid sequence of SEQ ID NO: 299, and a heavy chain comprising the amino acid sequence of SEQ ID NO: 300;
one or two light chains comprising the amino acid sequence of SEQ ID NO: 302, a heavy chain comprising the amino acid sequence of SEQ ID NO: 303, and a heavy chain comprising the amino acid sequence of SEQ ID NO: 304;
one or two light chains comprising the amino acid sequence of SEQ ID NO: 306, a heavy chain comprising the amino acid sequence of SEQ ID NO: 307, and a heavy chain comprising the amino acid sequence of SEQ ID NO: 308;
one or two light chains comprising the amino acid sequence of SEQ ID NO: 310, a heavy chain comprising the amino acid sequence of SEQ ID NO: 311, and a heavy chain comprising the amino acid sequence of SEQ ID NO: 312;
one or two light chains comprising the amino acid sequence of SEQ ID NO: 314, a heavy chain comprising the amino acid sequence of SEQ ID NO: 315, and a heavy chain comprising the amino acid sequence of SEQ ID NO: 316;
one or two light chains comprising the amino acid sequence of SEQ ID NO: 318, a heavy chain comprising the amino acid sequence of SEQ ID NO: 319, and a heavy chain comprising the amino acid sequence of SEQ ID NO: 320;
one or two light chains comprising the amino acid sequence of SEQ ID NO: 322, a heavy chain comprising the amino acid sequence of SEQ ID NO: 323, and a heavy chain comprising the amino acid sequence of SEQ ID NO: 324;
one or two light chains comprising the amino acid sequence of SEQ ID NO: 326, a heavy chain comprising the amino acid sequence of SEQ ID NO: 327, and a heavy chain comprising the amino acid sequence of SEQ ID NO: 328;
one or two light chains comprising the amino acid sequence of SEQ ID NO: 330, a heavy chain comprising the amino acid sequence of SEQ ID NO: 331, and a heavy chain comprising the amino acid sequence of SEQ ID NO: 332;
one or two light chains comprising the amino acid sequence of SEQ ID NO: 334, a heavy chain comprising the amino acid sequence of SEQ ID NO: 335, and a heavy chain comprising the amino acid sequence of SEQ ID NO: 336;
one or two light chains comprising the amino acid sequence of SEQ ID NO: 338, a heavy chain comprising the amino acid sequence of SEQ ID NO: 339, and a heavy chain comprising the amino acid sequence of SEQ ID NO: 340;
one or two light chains comprising the amino acid sequence of SEQ ID NO: 342, a heavy chain comprising the amino acid sequence of SEQ ID NO: 343, and a heavy chain comprising the amino acid sequence of SEQ ID NO: 344;
one or two light chains comprising the amino acid sequence of SEQ ID NO: 346, a heavy chain comprising the amino acid sequence of SEQ ID NO: 347, and a heavy chain comprising the amino acid sequence of SEQ ID NO: 348;
one or two light chains comprising the amino acid sequence of SEQ ID NO: 350, a heavy chain comprising the amino acid sequence of SEQ ID NO: 351, and a heavy chain comprising the amino acid sequence of SEQ ID NO: 352;
one or two light chains comprising the amino acid sequence of SEQ ID NO: 156, a heavy chain comprising the amino acid sequence of SEQ ID NO: 157, and a heavy chain comprising the amino acid sequence of SEQ ID NO: 217;
one or two light chains comprising the amino acid sequence of SEQ ID NO: 159, a heavy chain comprising the amino acid sequence of SEQ ID NO: 160, and a heavy chain comprising the amino acid sequence of SEQ ID NO: 218;
one or two light chains comprising the amino acid sequence of SEQ ID NO: 162, a heavy chain comprising the amino acid sequence of SEQ ID NO: 163, and a heavy chain comprising the amino acid sequence of SEQ ID NO: 219;
one or two light chains comprising the amino acid sequence of SEQ ID NO: 165, a heavy chain comprising the amino acid sequence of SEQ ID NO: 166, and a heavy chain comprising the amino acid sequence of SEQ ID NO: 220;
one or two light chains comprising the amino acid sequence of SEQ ID NO: 168, a heavy chain comprising the amino acid sequence of SEQ ID NO: 169, and a heavy chain comprising the amino acid sequence of SEQ ID NO: 221;
one or two light chains comprising the amino acid sequence of SEQ ID NO: 171, a heavy chain comprising the amino acid sequence of SEQ ID NO: 172, and a heavy chain comprising the amino acid sequence of SEQ ID NO: 222;
one or two light chains comprising the amino acid sequence of SEQ ID NO: 174, a heavy chain comprising the amino acid sequence of SEQ ID NO: 175, and a heavy chain comprising the amino acid sequence of SEQ ID NO: 223;
one or two light chains comprising the amino acid sequence of SEQ ID NO: 187, a heavy chain comprising the amino acid sequence of SEQ ID NO: 188, and a heavy chain comprising the amino acid sequence of SEQ ID NO: 224;
one or two light chains comprising the amino acid sequence of SEQ ID NO: 298, a heavy chain comprising the amino acid sequence of SEQ ID NO: 299, and a heavy chain comprising the amino acid sequence of SEQ ID NO: 301;
one or two light chains comprising the amino acid sequence of SEQ ID NO: 302, a heavy chain comprising the amino acid sequence of SEQ ID NO: 303, and a heavy chain comprising the amino acid sequence of SEQ ID NO: 305;
one or two light chains comprising the amino acid sequence of SEQ ID NO: 306, a heavy chain comprising the amino acid sequence of SEQ ID NO: 307, and a heavy chain comprising the amino acid sequence of SEQ ID NO: 309;
one or two light chains comprising the amino acid sequence of SEQ ID NO: 310, a heavy chain comprising the amino acid sequence of SEQ ID NO: 311, and a heavy chain comprising the amino acid sequence of SEQ ID NO: 313;
one or two light chains comprising the amino acid sequence of SEQ ID NO: 314, a heavy chain comprising the amino acid sequence of SEQ ID NO: 315, and a heavy chain comprising the amino acid sequence of SEQ ID NO: 317;
one or two light chains comprising the amino acid sequence of SEQ ID NO: 318, a heavy chain comprising the amino acid sequence of SEQ ID NO: 319, and a heavy chain comprising the amino acid sequence of SEQ ID NO: 321;
one or two light chains comprising the amino acid sequence of SEQ ID NO: 322, a heavy chain comprising the amino acid sequence of SEQ ID NO: 323, and a heavy chain comprising the amino acid sequence of SEQ ID NO: 325;
one or two light chains comprising the amino acid sequence of SEQ ID NO: 326, a heavy chain comprising the amino acid sequence of SEQ ID NO: 327, and a heavy chain comprising the amino acid sequence of SEQ ID NO: 329;
one or two light chains comprising the amino acid sequence of SEQ ID NO: 330, a heavy chain comprising the amino acid sequence of SEQ ID NO: 331, and a heavy chain comprising the amino acid sequence of SEQ ID NO: 333;
one or two light chains comprising the amino acid sequence of SEQ ID NO: 334, a heavy chain comprising the amino acid sequence of SEQ ID NO: 335, and a heavy chain comprising the amino acid sequence of SEQ ID NO: 337;
one or two light chains comprising the amino acid sequence of SEQ ID NO: 338, a heavy chain comprising the amino acid sequence of SEQ ID NO: 339, and a heavy chain comprising the amino acid sequence of SEQ ID NO: 341;
one or two light chains comprising the amino acid sequence of SEQ ID NO: 342, a heavy chain comprising the amino acid sequence of SEQ ID NO: 343, and a heavy chain comprising the amino acid sequence of SEQ ID NO: 345;
one or two light chains comprising the amino acid sequence of SEQ ID NO: 346, a heavy chain comprising the amino acid sequence of SEQ ID NO: 347, and a heavy chain comprising the amino acid sequence of SEQ ID NO: 349; or one or two light chains comprising the amino acid sequence of SEQ ID NO: 350, a heavy chain comprising the amino acid sequence of SEQ ID NO: 351, and a heavy chain comprising the amino acid sequence of SEQ ID NO: 353.

Embodiment 136. The kit or the composition of any one of Embodiments 76-135, wherein the IL-2 fusion protein comprises four polypeptide chains, wherein: the first polypeptide chain comprises the amino acid sequence of SEQ ID NO: 334, the second polypeptide chain comprises the amino acid sequence of SEQ ID NO: 335, the third polypeptide chain comprises the amino acid sequence of SEQ ID NO: 336, and the fourth polypeptide chain comprises the amino acid sequence of SEQ ID NO: 334;
the first polypeptide chain comprises the amino acid sequence of SEQ ID NO: 334, the second polypeptide chain comprises the amino acid sequence of SEQ ID NO: 335, the third polypeptide chain comprises the amino acid sequence of SEQ ID NO: 337, and the fourth polypeptide chain comprises the amino acid sequence of SEQ ID NO: 334;
the first polypeptide chain comprises the amino acid sequence of SEQ ID NO: 338, the second polypeptide chain comprises the amino acid sequence of SEQ ID NO: 339, the third polypeptide chain comprises the amino acid sequence of SEQ ID NO: 340, and the fourth polypeptide chain comprises the amino acid sequence of SEQ ID NO: 338; or
the first polypeptide chain comprises the amino acid sequence of SEQ ID NO: 338, the second polypeptide chain comprises the amino acid sequence of SEQ ID NO: 339, the third polypeptide chain comprises the amino acid sequence of SEQ ID NO: 341, and the fourth polypeptide chain comprises the amino acid sequence of SEQ ID NO: 338.

Embodiment 137. The kit or the composition of any one of Embodiments 76-136, or 107-134, wherein the IL-21 fusion protein comprises:
one or two light chains comprising the amino acid sequence of SEQ ID NO: 557, a heavy chain comprising the amino acid sequence of SEQ ID NO: 558, and a heavy chain comprising the amino acid sequence of SEQ ID NO: 559;
one or two light chains comprising the amino acid sequence of SEQ ID NO: 561, a heavy chain comprising the amino acid sequence of SEQ ID NO: 562, and a heavy chain comprising the amino acid sequence of SEQ ID NO: 563;
one or two light chains comprising the amino acid sequence of SEQ ID NO: 565, a heavy chain comprising the amino acid sequence of SEQ ID NO: 566, and a heavy chain comprising the amino acid sequence of SEQ ID NO: 567;
one or two light chains comprising the amino acid sequence of SEQ ID NO: 569, a heavy chain comprising the amino acid sequence of SEQ ID NO: 570, and a heavy chain comprising the amino acid sequence of SEQ ID NO: 571;
one or two light chains comprising the amino acid sequence of SEQ ID NO: 573, a heavy chain comprising the amino acid sequence of SEQ ID NO: 574, and a heavy chain comprising the amino acid sequence of SEQ ID NO: 575;
one or two light chains comprising the amino acid sequence of SEQ ID NO: 577, a heavy chain comprising the amino acid sequence of SEQ ID NO: 578, and a heavy chain comprising the amino acid sequence of SEQ ID NO: 579;
one or two light chains comprising the amino acid sequence of SEQ ID NO: 581, a heavy chain comprising the amino acid sequence of SEQ ID NO: 582, and a heavy chain comprising the amino acid sequence of SEQ ID NO: 583;
one or two light chains comprising the amino acid sequence of SEQ ID NO: 585, a heavy chain comprising the amino acid sequence of SEQ ID NO: 586, and a heavy chain comprising the amino acid sequence of SEQ ID NO: 587;
one or two light chains comprising the amino acid sequence of SEQ ID NO: 589, a heavy chain comprising the amino acid sequence of SEQ ID NO: 590, and a heavy chain comprising the amino acid sequence of SEQ ID NO: 591;
one or two light chains comprising the amino acid sequence of SEQ ID NO: 557, a heavy chain comprising the amino acid sequence of SEQ ID NO: 558, and a heavy chain comprising the amino acid sequence of SEQ ID NO: 560;
one or two light chains comprising the amino acid sequence of SEQ ID NO: 561, a heavy chain comprising the amino acid sequence of SEQ ID NO: 562, and a heavy chain comprising the amino acid sequence of SEQ ID NO: 564;
one or two light chains comprising the amino acid sequence of SEQ ID NO: 565, a heavy chain comprising the amino acid sequence of SEQ ID NO: 566, and a heavy chain comprising the amino acid sequence of SEQ ID NO: 568;
one or two light chains comprising the amino acid sequence of SEQ ID NO: 569, a heavy chain comprising the amino acid sequence of SEQ ID NO: 570, and a heavy chain comprising the amino acid sequence of SEQ ID NO: 572;
one or two light chains comprising the amino acid sequence of SEQ ID NO: 573, a heavy chain comprising the amino acid sequence of SEQ ID NO: 574, and a heavy chain comprising the amino acid sequence of SEQ ID NO: 576;
one or two light chains comprising the amino acid sequence of SEQ ID NO: 577, a heavy chain comprising the amino acid sequence of SEQ ID NO: 578, and a heavy chain comprising the amino acid sequence of SEQ ID NO: 580;
one or two light chains comprising the amino acid sequence of SEQ ID NO: 581, a heavy chain comprising the amino acid sequence of SEQ ID NO: 582, and a heavy chain comprising the amino acid sequence of SEQ ID NO: 584;
one or two light chains comprising the amino acid sequence of SEQ ID NO: 585, a heavy chain comprising the amino acid sequence of SEQ ID NO: 586, and a heavy chain comprising the amino acid sequence of SEQ ID NO: 588; or one or two light chains comprising the amino acid sequence of SEQ ID NO: 589, a heavy chain comprising the amino acid sequence of SEQ ID NO: 590, and a heavy chain comprising the amino acid sequence of SEQ ID NO: 592.

Embodiment 138. The kit or the composition of any one of Embodiments 76-137, or 107-134, wherein the IL-21 fusion protein comprises four polypeptide chains, wherein:
the first polypeptide chain comprises the amino acid sequence of SEQ ID NO: 557, the second polypeptide chain comprises the amino acid sequence of SEQ ID NO: 558, the third polypeptide chain comprises the amino acid sequence of SEQ ID NO: 559, and the fourth polypeptide chain comprises the amino acid sequence of SEQ ID NO: 557;
the first polypeptide chain comprises the amino acid sequence of SEQ ID NO: 557, the second polypeptide chain comprises the amino acid sequence of SEQ ID NO: 558, the third polypeptide chain comprises the amino acid sequence of SEQ ID NO: 560, and the fourth polypeptide chain comprises the amino acid sequence of SEQ ID NO: 557;
the first polypeptide chain comprises the amino acid sequence of SEQ ID NO: 561, the second polypeptide chain comprises the amino acid sequence of SEQ ID NO: 562, the third polypeptide chain comprises the amino acid sequence of SEQ ID NO: 563, and the fourth polypeptide chain comprises the amino acid sequence of SEQ ID NO: 561;
the first polypeptide chain comprises the amino acid sequence of SEQ ID NO: 561, the second polypeptide chain comprises the amino acid sequence of SEQ ID NO: 562, the third polypeptide chain comprises the amino acid sequence of SEQ ID NO: 564, and the fourth polypeptide chain comprises the amino acid sequence of SEQ ID NO: 561;
the first polypeptide chain comprises the amino acid sequence of SEQ ID NO: 565, the second polypeptide chain comprises the amino acid sequence of SEQ ID NO: 566, the third polypeptide chain comprises the amino acid sequence of SEQ ID NO: 567, and the fourth polypeptide chain comprises the amino acid sequence of SEQ ID NO: 565;
the first polypeptide chain comprises the amino acid sequence of SEQ ID NO: 565, the second polypeptide chain comprises the amino acid sequence of SEQ ID NO: 566, the third polypeptide chain comprises the amino acid sequence of SEQ ID NO: 568, and the fourth polypeptide chain comprises the amino acid sequence of SEQ ID NO: 565;
the first polypeptide chain comprises the amino acid sequence of SEQ ID NO: 569, the second polypeptide chain comprises the amino acid sequence of SEQ ID NO: 570, the third polypeptide chain comprises the amino acid sequence of SEQ ID NO: 571, and the fourth polypeptide chain comprises the amino acid sequence of SEQ ID NO: 569;
the first polypeptide chain comprises the amino acid sequence of SEQ ID NO: 569, the second polypeptide chain comprises the amino acid sequence of SEQ ID NO: 570, the third polypeptide chain comprises the amino acid sequence of SEQ ID NO: 572, and the fourth polypeptide chain comprises the amino acid sequence of SEQ ID NO: 569;
the first polypeptide chain comprises the amino acid sequence of SEQ ID NO: 573, the second polypeptide chain comprises the amino acid sequence of SEQ ID NO: 574, the third polypeptide chain comprises the amino acid sequence of SEQ ID NO: 575, and the fourth polypeptide chain comprises the amino acid sequence of SEQ ID NO: 573;
the first polypeptide chain comprises the amino acid sequence of SEQ ID NO: 573, the second polypeptide chain comprises the amino acid sequence of SEQ ID NO: 574, the third polypeptide chain comprises the amino acid sequence of SEQ ID NO: 576, and the fourth polypeptide chain comprises the amino acid sequence of SEQ ID NO: 573;
the first polypeptide chain comprises the amino acid sequence of SEQ ID NO: 577, the second polypeptide chain comprises the amino acid sequence of SEQ ID NO: 578, the third polypeptide chain comprises the amino acid sequence of SEQ ID NO: 579, and the fourth polypeptide chain comprises the amino acid sequence of SEQ ID NO: 577;
the first polypeptide chain comprises the amino acid sequence of SEQ ID NO: 577, the second polypeptide chain comprises the amino acid sequence of SEQ ID NO: 578, the third polypeptide chain comprises the amino acid sequence of SEQ ID NO: 580, and the fourth polypeptide chain comprises the amino acid sequence of SEQ ID NO: 577;
the first polypeptide chain comprises the amino acid sequence of SEQ ID NO: 581, the second polypeptide chain comprises the amino acid sequence of SEQ ID NO: 582, the third polypeptide chain comprises the amino acid sequence of SEQ ID NO: 583, and the fourth polypeptide chain comprises the amino acid sequence of SEQ ID NO: 581;
the first polypeptide chain comprises the amino acid sequence of SEQ ID NO: 581, the second polypeptide chain comprises the amino acid sequence of SEQ ID NO: 582, the third polypeptide chain comprises the amino acid sequence of SEQ ID NO: 584, and the fourth polypeptide chain comprises the amino acid sequence of SEQ ID NO: 581;
the first polypeptide chain comprises the amino acid sequence of SEQ ID NO: 585, the second polypeptide chain comprises the amino acid sequence of SEQ ID NO: 586, the third polypeptide chain comprises the amino acid sequence of SEQ ID NO: 587, and the fourth polypeptide chain comprises the amino acid sequence of SEQ ID NO: 585;
the first polypeptide chain comprises the amino acid sequence of SEQ ID NO: 585, the second polypeptide chain comprises the amino acid sequence of SEQ ID NO: 586, the third polypeptide chain comprises the amino acid sequence of SEQ ID NO: 588, and the fourth polypeptide chain comprises the amino acid sequence of SEQ ID NO: 585;
the first polypeptide chain comprises the amino acid sequence of SEQ ID NO: 589, the second polypeptide chain comprises the amino acid sequence of SEQ ID NO: 590, the third polypeptide chain comprises the amino acid sequence of SEQ ID NO: 591, and the fourth polypeptide chain comprises the amino acid sequence of SEQ ID NO: 589; or
the first polypeptide chain comprises the amino acid sequence of SEQ ID NO: 589, the second polypeptide chain comprises the amino acid sequence of SEQ ID NO: 590, the third polypeptide chain comprises the amino acid sequence of SEQ ID NO: 592, and the fourth polypeptide chain comprises the amino acid sequence of SEQ ID NO: 589.

Embodiment 139. The kit or the composition of any one of Embodiments 76 or 79-138, wherein the IL-21 fusion protein is administered prior to the administration of the IL-2 fusion protein.

Embodiment 140. The kit or the composition of any one of Embodiments 76 or 79-138, wherein the IL-2 fusion protein is administered prior to the administration of the IL-21 fusion protein.

Embodiment 141. The kit or the composition of any one of Embodiments 76 or 79-138, wherein the IL-21 fusion protein and IL-2 fusion protein are administered concurrently, sequentially, or simultaneously.

Embodiment 142. The kit or the composition of any one of Embodiments 76 or 79-141, wherein the IL-21 fusion protein is formulated in a pharmaceutical composition comprising a pharmaceutically acceptable carrier.

Embodiment 143. The kit or the composition of any one of Embodiments 76 or 79-142, wherein the IL-2 fusion protein is formulated in a pharmaceutical composition comprising a pharmaceutically acceptable carrier.

Embodiment 144. The kit of Embodiment 142 or 143, wherein the IL-21 fusion protein and the IL-2 fusion protein are formulated in a single pharmaceutical composition.

Embodiment 145. The kit of Embodiment 142 or 143, wherein the IL-21 fusion protein and the IL-2 fusion protein are formulated in separate pharmaceutical compositions.

Embodiment 146. The kit or the composition of any one of Embodiments 76-145, wherein the first antibody or antigen binding fragment thereof and the second antibody or antigen binding fragment thereof each target CD8ab or CD8b.

Embodiment 147. The kit of Embodiment 146, wherein the first antibody or antigen binding fragment thereof and the second antibody or antigen binding fragment thereof each targets a different epitope on CD8ab or CD8b.

Embodiment 148. The kit of Embodiment 146 or 147, wherein the first antibody or antigen binding fragment thereof and the second antibody or antigen binding fragment thereof do not cross compete for binding to CD8ab or CD8b.

Embodiment 149. An IL-21 fusion protein for use in treating a subject in need thereof, wherein the subject has received or is receiving treatment with an IL-2 fusion protein, wherein the IL-21 fusion protein comprising (i) a first antibody or antigen-binding fragment thereof that specifically binds to human CD8, and (ii) an IL-21 polypeptide, and wherein the IL-2 fusion protein comprising (i) an second antibody or antigen-binding fragment thereof that specifically binds human CD8, and (ii) an IL-2 polypeptide.

Embodiment 150. An IL-2 fusion protein for use in treating a subject in need thereof, wherein the subject has received or is receiving treatment with an IL-21 fusion protein, wherein the IL-21 fusion protein comprising (i) a first antibody or antigen-binding fragment thereof that specifically binds to human CD8, and (ii) an IL-21 polypeptide, and wherein the IL-2 fusion protein comprising (i) an second antibody or antigen-binding fragment thereof that specifically binds human CD8, and (ii) an IL-2 polypeptide.

Embodiment 151. An IL-2 fusion protein and an IL-21 fusion protein for use in treating a subject in need thereof, wherein
(a) the IL-21 fusion protein comprising (i) a first antibody or antigen-binding fragment thereof that specifically binds to human CD8, and (ii) an IL-21 polypeptide; and (b) the IL-2 fusion protein comprising (i) an second antibody or antigen-binding fragment thereof that specifically binds human CD8, and (ii) an IL-2 polypeptide.

Embodiment 152. A pharmaceutical composition comprising the composition of any one of Embodiments 86-148 and a pharmaceutically acceptable carrier.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

1. A method of treating a subject having a disease, the method comprising administering to the subject:
(a) an effective amount of an IL-21 fusion protein comprising (i) a first antibody or antigen-binding fragment thereof that specifically binds to human CD8, and (ii) an IL-21 polypeptide; and
(b) an effective amount of an IL-2 fusion protein comprising (i) a second antibody or antigen-binding fragment thereof that specifically binds human CD8, and (ii) an IL-2 polypeptide.

2. (canceled)

3. (canceled)

4. The method of claim 1, wherein the disease comprises a cancer or an infectious disease.

5.-9. (canceled)

10. The method of claim 1, wherein the first antibody or antigen-binding fragment thereof and the second antibody or antigen-binding fragment thereof are same.

11. (canceled)

12. The method of claim 1, wherein the first antibody or antigen-binding fragment thereof comprises a heavy chain and a light chain, wherein the heavy chain comprises a heavy chain variable (VH) domain; and the light chain comprises a light chain variable (VL) domain, and wherein in the first antibody or antigen-binding fragment thereof:
the VH domain comprises a CDR-H1 comprising the amino acid sequence of SEQ ID NO: 13, a CDR-H2 comprising the amino acid sequence of SEQ ID NO: 14, and a CDR-H3 comprising the amino acid sequence of SEQ ID NO: 15; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 18;
the VH domain comprises a CDR-H1 comprising the amino acid sequence of SEQ ID NO: 19, a CDR-H2 comprising the amino acid sequence of SEQ ID NO: 20, and a CDR-H3 comprising the amino acid sequence of SEQ ID NO: 21; and the VL domain comprises a CDR-L1 comprising the amino acid sequence of SEQ ID NO: 22, a CDR-L2 comprising the amino acid sequence of SEQ ID NO: 23, and a CDR-L3 comprising the amino acid sequence of SEQ ID NO: 24;
the VH domain comprises a CDR-H1 comprising the amino acid sequence of SEQ ID NO: 25, a CDR-H2 comprising the amino acid sequence of SEQ ID NO: 26, and a CDR-H3 comprising the amino acid sequence of SEQ ID NO: 27; and the VL domain comprises a CDR-L1 comprising the amino acid sequence of SEQ ID NO: 28, a CDR-L2 comprising the amino acid sequence of SEQ ID NO: 29, and a CDR-L3 comprising the amino acid sequence of SEQ ID NO: 30;
the VH domain comprises a CDR-H1 comprising the amino acid sequence of SEQ ID NO: 31, a CDR-H2 comprising the amino acid sequence of SEQ ID NO: 32, and a CDR-H3 comprising the amino acid sequence of SEQ ID NO: 33; and the VL domain comprises a CDR-L1 comprising the amino acid sequence of SEQ ID NO: 34, a CDR-L2 comprising the amino acid sequence of SEQ ID NO: 35, and a CDR-L3 comprising the amino acid sequence of SEQ ID NO: 36;
the VH domain comprises a CDR-H1 comprising the amino acid sequence of SEQ ID NO: 37, a CDR-H2 comprising the amino acid sequence of SEQ ID NO: 38, and a CDR-H3 comprising the amino acid sequence of SEQ ID NO: 39; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
the VH domain comprises a CDR-H1 comprising the amino acid sequence of SEQ ID NO: 43, a CDR-H2 comprising the amino acid sequence of SEQ ID NO: 44, and a CDR-H3 comprising the amino acid sequence of SEQ ID NO: 45; and the VL domain comprises a CDR-L1 comprising the amino acid sequence of SEQ ID NO: 46, a CDR-L2 comprising the amino acid sequence of SEQ ID NO: 47, and a CDR-L3 comprising the amino acid sequence of SEQ ID NO: 48;
the VH domain comprises a CDR-H1 comprising the amino acid sequence of SEQ ID NO: 1, a CDR-H2 comprising the amino acid sequence of SEQ ID NO: 2, and a CDR-H3 comprising the amino acid sequence of SEQ ID NO: 3; and the VL domain comprises a CDR-L1 comprising the amino acid sequence of SEQ ID NO: 4, a CDR-L2 comprising the amino acid sequence of SEQ ID NO: 5, and a CDR-L3 comprising the amino acid sequence of SEQ ID NO: 6;
the VH domain comprises a CDR-H1 comprising the amino acid sequence of SEQ ID NO: 177, a CDR-H2 comprising the amino acid sequence of SEQ ID NO: 178, and a CDR-H3 comprising the amino acid sequence of SEQ ID NO: 179; and the VL domain comprises a CDR-L1 comprising the amino acid sequence of SEQ ID NO: 180, a CDR-L2 comprising the amino acid sequence of SEQ ID NO: 181, and a CDR-L3 comprising the amino acid sequence of SEQ ID NO: 182;
the VH domain comprises a CDR-H1 comprising the amino acid sequence of SEQ ID NO: 225, a CDR-H2 comprising the amino acid sequence of SEQ ID NO: 226, and a CDR-H3 comprising the amino acid sequence of SEQ ID NO: 227; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228;
the VH domain comprises a CDR-H1 comprising the amino acid sequence of SEQ ID NO: 229, a CDR-H2 comprising the amino acid sequence of SEQ ID NO: 230, and a CDR-H3 comprising the amino acid sequence of SEQ ID NO: 231; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
the VH domain comprises a CDR-H1 comprising the amino acid sequence of SEQ ID NO: 225, a CDR-H2 comprising the amino acid sequence of SEQ ID NO: 232, and a CDR-H3 comprising the amino acid sequence of SEQ ID NO: 233; and the VL domain comprises a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236;
the VH domain comprises a CDR-H1 comprising the amino acid sequence of SEQ ID NO: 225, a CDR-H2 comprising the amino acid sequence of SEQ ID NO: 232, and a CDR-H3 comprising the amino acid sequence of SEQ ID NO: 233; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228;
the VH domain comprises a CDR-H1 comprising the amino acid sequence of SEQ ID NO: 229, a CDR-H2 comprising the amino acid sequence of SEQ ID NO: 237, and a CDR-H3 comprising the amino acid sequence of SEQ ID NO: 231; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42; or
the VH domain comprises a CDR-H1 comprising the amino acid sequence of SEQ ID NO: 51, a CDR-H2 comprising the amino acid sequence of SEQ ID NO: 52, and a CDR-H3 comprising the amino acid sequence of SEQ ID NO: 15; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 18;
the VH domain comprises a CDR-H1 comprising the amino acid sequence of SEQ ID NO: 53, a CDR-H2 comprising the amino acid sequence of SEQ ID NO: 52, and a CDR-H3 comprising the amino acid sequence of SEQ ID NO: 21; and the VL domain comprises a CDR-L1 comprising the amino acid sequence of SEQ ID NO: 22, a CDR-L2 comprising the amino acid sequence of SEQ ID NO: 23, and a CDR-L3 comprising the amino acid sequence of SEQ ID NO: 24;
the VH domain comprises a CDR-H1 comprising the amino acid sequence of SEQ ID NO: 49, a CDR-H2 comprising the amino acid sequence of SEQ ID NO: 52, and a CDR-H3 comprising the amino acid sequence of SEQ ID NO: 27; and the VL domain comprises a CDR-L1 comprising the amino acid sequence of SEQ ID NO: 28, a CDR-L2 comprising the amino acid sequence of SEQ ID NO: 29, and a CDR-L3 comprising the amino acid sequence of SEQ ID NO: 30;
the VH domain comprises a CDR-H1 comprising the amino acid sequence of SEQ ID NO: 54, a CDR-H2 comprising the amino acid sequence of SEQ ID NO: 52, and a CDR-H3 comprising the amino acid sequence of SEQ ID NO: 33; and the VL domain comprises a CDR-L1 comprising the amino acid sequence of SEQ ID NO: 34, a CDR-L2 comprising the amino acid sequence of SEQ ID NO: 35, and a CDR-L3 comprising the amino acid sequence of SEQ ID NO: 36;
the VH domain comprises a CDR-H1 comprising the amino acid sequence of SEQ ID NO: 55, a CDR-H2 comprising the amino acid sequence of SEQ ID NO: 56, and a CDR-H3 comprising the amino acid sequence of SEQ ID NO: 39; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
the VH domain comprises a CDR-H1 comprising the amino acid sequence of SEQ ID NO: 55, a CDR-H2 comprising the amino acid sequence of SEQ ID NO: 57, and a CDR-H3 comprising the amino acid sequence of SEQ ID NO: 45; and the VL domain comprises a CDR-L1 comprising the amino acid sequence of SEQ ID NO: 46, a CDR-L2 comprising the amino acid sequence of SEQ ID NO: 47, and a CDR-L3 comprising the amino acid sequence of SEQ ID NO: 48;
the VH domain comprises a CDR-H1 comprising the amino acid sequence of SEQ ID NO: 49, a CDR-H2 comprising the amino acid sequence of SEQ ID NO: 50, and a CDR-H3 comprising the amino acid sequence of SEQ ID NO: 3; and the VL domain comprises a CDR-L1 comprising the amino acid sequence of SEQ ID NO: 4, a CDR-L2 comprising the amino acid sequence of SEQ ID NO: 5, and a CDR-L3 comprising the amino acid sequence of SEQ ID NO;
the VH domain comprises a CDR-H1 comprising the amino acid sequence of SEQ ID NO: 183, a CDR-H2 comprising the amino acid sequence of SEQ ID NO: 184, and a CDR-H3 comprising the amino acid sequence of SEQ ID NO: 179; and the VL domain comprises a CDR-L1 comprising the amino acid sequence of SEQ ID NO: 180, a CDR-L2 comprising the amino acid sequence of SEQ ID NO: 181, and a CDR-L3 comprising the amino acid sequence of SEQ ID NO: 182;
the VH domain comprises a CDR-H1 comprising the amino acid sequence of SEQ ID NO: 238, a CDR-H2 comprising the amino acid sequence of SEQ ID NO: 239, and a CDR-H3 comprising the amino acid sequence of SEQ ID NO: 233; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228;
the VH domain comprises a CDR-H1 comprising the amino acid sequence of SEQ ID NO: 240, a CDR-H2 comprising the amino acid sequence of SEQ ID NO: 241, and a CDR-H3 comprising the amino acid sequence of SEQ ID NO: 242; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
the VH domain comprises a CDR-H1 comprising the amino acid sequence of SEQ ID NO: 238, a CDR-H2 comprising the amino acid sequence of SEQ ID NO: 243, and a CDR-H3 comprising the amino acid sequence of SEQ ID NO: 233; and the VL domain comprises a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236;
the VH domain comprises a CDR-H1 comprising the amino acid sequence of SEQ ID NO: 238, a CDR-H2 comprising the amino acid sequence of SEQ ID NO: 243, and a CDR-H3 comprising the amino acid sequence of SEQ ID NO: 233; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228: or
the VH domain comprises a CDR-H1 comprising the amino acid sequence of SEQ ID NO: 240, a CDR-H2 comprising the amino acid sequence of SEQ ID NO: 244, and a CDR-H3 comprising the amino acid sequence of SEQ ID NO: 242; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42.

13. (canceled)

14. The method of claim 1, wherein the first antibody or antigen-binding fragment thereof comprises a heavy chain and a light chain, wherein the heavy chain comprises a heavy chain variable (VH) domain, and the light chain comprises a light chain variable (VL) domain, and wherein the VH and VL domains of the first antibody or antigen-binding fragment thereof comprise the amino acid sequences of:
(a) SEQ ID NOs: 58 and 59, respectively;
(b) SEQ ID NOs: 247 and 248, respectively;
(c) SEQ ID NOs: 62 and 63, respectively;
(d) SEQ ID NOs: 64 and 65, respectively;
(e) SEQ ID NOs: 66 and 67, respectively;
(f) SEQ ID NOs: 68 and 69, respectively;
(g) SEQ ID NOs: 70 and 71, respectively;
(h) SEQ ID NOs: 72 and 73, respectively;
(i) SEQ ID NOs: 185 and 186, respectively;
(j) SEQ ID NOs: 245 and 246, respectively;
(k) SEQ ID NOs: 249 and 250, respectively;
(l) SEQ ID NOs: 251 and 252, respectively;
(m) SEQ ID NOs: 253 and 254, respectively;
(n) SEQ ID NOs: 255 and 256, respectively; or
(o) SEQ ID NOs: 257 and 258, respectively.

15. The method of claim 1, wherein the second antibody or antigen-binding fragment thereof comprises a heavy chain and a light chain, wherein the heavy chain comprises a heavy chain variable (VH) domain; and the light chain comprises a light chain variable (VL) domain, and wherein in the second antibody or antigen-binding fragment thereof:
the VH domain comprises a CDR-H1 comprising the amino acid sequence of SEQ ID NO: 13, a CDR-H2 comprising the amino acid sequence of SEQ ID NO: 14, and a CDR-H3 comprising the amino acid sequence of SEQ ID NO: 15; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 18;
the VH domain comprises a CDR-H1 comprising the amino acid sequence of SEQ ID NO: 19, a CDR-H2 comprising the amino acid sequence of SEQ ID NO: 20, and a CDR-H3 comprising the amino acid sequence of SEQ ID NO: 21; and the VL domain comprises a CDR-L1 comprising the amino acid sequence of SEQ ID NO: 22, a CDR-L2 comprising the amino acid sequence of SEQ ID NO: 23, and a CDR-L3 comprising the amino acid sequence of SEQ ID NO: 24;
the VH domain comprises a CDR-H1 comprising the amino acid sequence of SEQ ID NO: 25, a CDR-H2 comprising the amino acid sequence of SEQ ID NO: 26, and a CDR-H3 comprising the amino acid sequence of SEQ ID NO: 27; and the VL domain comprises a CDR-L1 comprising the amino acid sequence of SEQ ID NO: 28, a CDR-L2 comprising the amino acid sequence of SEQ ID NO: 29, and a CDR-L3 comprising the amino acid sequence of SEQ ID NO: 30;
the VH domain comprises a CDR-H1 comprising the amino acid sequence of SEQ ID NO: 31, a CDR-H2 comprising the amino acid sequence of SEQ ID NO: 32, and a CDR-H3 comprising the amino acid sequence of SEQ ID NO: 33; and the VL domain comprises a CDR-L1 comprising the amino acid sequence of SEQ ID NO: 34, a CDR-L2 comprising the amino acid sequence of SEQ ID NO: 35, and a CDR-L3 comprising the amino acid sequence of SEQ ID NO: 36;
the VH domain comprises a CDR-H1 comprising the amino acid sequence of SEQ ID NO: 37, a CDR-H2 comprising the amino acid sequence of SEQ ID NO: 38, and a CDR-H3 comprising the amino acid sequence of SEQ ID NO: 39; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
the VH domain comprises a CDR-H1 comprising the amino acid sequence of SEQ ID NO: 43, a CDR-H2 comprising the amino acid sequence of SEQ ID NO: 44, and a CDR-H3 comprising the amino acid sequence of SEQ ID NO: 45; and the VL domain comprises a CDR-L1 comprising the amino acid sequence of SEQ ID NO: 46, a CDR-L2 comprising the amino acid sequence of SEQ ID NO: 47, and a CDR-L3 comprising the amino acid sequence of SEQ ID NO: 48;
the VH domain comprises a CDR-H1 comprising the amino acid sequence of SEQ ID NO: 1, a CDR-H2 comprising the amino acid sequence of SEQ ID NO: 2, and a CDR-H3 comprising the amino acid sequence of SEQ ID NO: 3; and the VL domain comprises a CDR-L1 comprising the amino acid sequence of SEQ ID NO: 4, a CDR-L2 comprising the amino acid sequence of SEQ ID NO: 5, and a CDR-L3 comprising the amino acid sequence of SEQ ID NO: 6;
the VH domain comprises a CDR-H1 comprising the amino acid sequence of SEQ ID NO: 177, a CDR-H2 comprising the amino acid sequence of SEQ ID NO: 178, and a CDR-H3 comprising the amino acid sequence of SEQ ID NO: 179; and the VL domain comprises a CDR-L1 comprising the amino acid sequence of SEQ ID NO: 180, a CDR-L2 comprising the amino acid sequence of SEQ ID NO: 181, and a CDR-L3 comprising the amino acid sequence of SEQ ID NO: 182;
the VH domain comprises a CDR-H1 comprising the amino acid sequence of SEQ ID NO: 225, a CDR-H2 comprising the amino acid sequence of SEQ ID NO: 226, and a CDR-H3 comprising the amino acid sequence of SEQ ID NO: 227; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228;
the VH domain comprises a CDR-H1 comprising the amino acid sequence of SEQ ID NO: 229, a CDR-H2 comprising the amino acid sequence of SEQ ID NO: 230, and a CDR-H3 comprising the amino acid sequence of SEQ ID NO: 231; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
the VH domain comprises a CDR-H1 comprising the amino acid sequence of SEQ ID NO: 225, a CDR-H2 comprising the amino acid sequence of SEQ ID NO: 232, and a CDR-H3 comprising the amino acid sequence of SEQ ID NO: 233; and the VL domain comprises a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236;
the VH domain comprises a CDR-H1 comprising the amino acid sequence of SEQ ID NO: 225, a CDR-H2 comprising the amino acid sequence of SEQ ID NO: 232, and a CDR-H3 comprising the amino acid sequence of SEQ ID NO: 233; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228;
the VH domain comprises a CDR-H1 comprising the amino acid sequence of SEQ ID NO: 229, a CDR-H2 comprising the amino acid sequence of SEQ ID NO: 237, and a CDR-H3 comprising the amino acid sequence of SEQ ID NO: 231; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
the VH domain comprises a CDR-H1 comprising the amino acid sequence of SEQ ID NO: 51, a CDR-H2 comprising the amino acid sequence of SEQ ID NO: 52, and a CDR-H3 comprising the amino acid sequence of SEQ ID NO: 15; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 18;
the VH domain comprises a CDR-H1 comprising the amino acid sequence of SEQ ID NO: 53, a CDR-H2 comprising the amino acid sequence of SEQ ID NO: 52, and a CDR-H3 comprising the amino acid sequence of SEQ ID NO: 21; and the VL domain comprises a CDR-L1 comprising the amino acid sequence of SEQ ID NO: 22, a CDR-L2 comprising the amino acid sequence of SEQ ID NO: 23, and a CDR-L3 comprising the amino acid sequence of SEQ ID NO: 24;
the VH domain comprises a CDR-H1 comprising the amino acid sequence of SEQ ID NO: 49, a CDR-H2 comprising the amino acid sequence of SEQ ID NO: 52, and a CDR-H3 comprising the amino acid sequence of SEQ ID NO: 27; and the VL domain comprises a CDR-L1 comprising the amino acid sequence of SEQ ID NO: 28, a CDR-L2 comprising the amino acid sequence of SEQ ID NO: 29, and a CDR-L3 comprising the amino acid sequence of SEQ ID NO: 30;
the VH domain comprises a CDR-H1 comprising the amino acid sequence of SEQ ID NO: 54, a CDR-H2 comprising the amino acid sequence of SEQ ID NO: 52, and a CDR-H3 comprising the amino acid sequence of SEQ ID NO: 33; and the VL domain comprises a CDR-L1 comprising the amino acid sequence of SEQ ID NO: 34, a CDR-L2 comprising the amino acid sequence of SEQ ID NO: 35, and a CDR-L3 comprising the amino acid sequence of SEQ ID NO: 36;
the VH domain comprises a CDR-H1 comprising the amino acid sequence of SEQ ID NO: 55, a CDR-H2 comprising the amino acid sequence of SEQ ID NO: 56, and a CDR-H3 comprising the amino acid sequence of SEQ ID NO: 39; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
the VH domain comprises a CDR-H1 comprising the amino acid sequence of SEQ ID NO: 55, a CDR-H2 comprising the amino acid sequence of SEQ ID NO: 57, and a CDR-H3 comprising the amino acid sequence of SEQ ID NO: 45; and the VL domain comprises a CDR-L1 comprising the amino acid sequence of SEQ ID NO: 46, a CDR-L2 comprising the amino acid sequence of SEQ ID NO: 47, and a CDR-L3 comprising the amino acid sequence of SEQ ID NO: 48;
the VH domain comprises a CDR-H1 comprising the amino acid sequence of SEQ ID NO: 49, a CDR-H2 comprising the amino acid sequence of SEQ ID NO: 50, and a CDR-H3 comprising the amino acid sequence of SEQ ID NO: 3; and the VL domain comprises a CDR-L1 comprising the amino acid sequence of SEQ ID NO: 4, a CDR-L2 comprising the amino acid sequence of SEQ ID NO: 5, and a CDR-L3 comprising the amino acid sequence of SEQ ID NO;
the VH domain comprises a CDR-H1 comprising the amino acid sequence of SEQ ID NO: 183, a CDR-H2 comprising the amino acid sequence of SEQ ID NO: 184, and a CDR-H3 comprising the amino acid sequence of SEQ ID NO: 179; and the VL domain comprises a CDR-L1 comprising the amino acid sequence of SEQ ID NO: 180, a CDR-L2 comprising the amino acid sequence of SEQ ID NO: 181, and a CDR-L3 comprising the amino acid sequence of SEQ ID NO: 182;
the VH domain comprises a CDR-H1 comprising the amino acid sequence of SEQ ID NO: 238, a CDR-H2 comprising the amino acid sequence of SEQ ID NO: 239, and a CDR-H3 comprising the amino acid sequence of SEQ ID NO: 233; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228;
the VH domain comprises a CDR-H1 comprising the amino acid sequence of SEQ ID NO: 240, a CDR-H2 comprising the amino acid sequence of SEQ ID NO: 241, and a CDR-H3 comprising the amino acid sequence of SEQ ID NO: 242; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42;
the VH domain comprises a CDR-H1 comprising the amino acid sequence of SEQ ID NO: 238, a CDR-H2 comprising the amino acid sequence of SEQ ID NO: 243, and a CDR-H3 comprising the amino acid sequence of SEQ ID NO: 233; and the VL domain comprises a CDR-L1 comprising the amino acid sequence of SEQ ID NO: 234, a CDR-L2 comprising the amino acid sequence of SEQ ID NO: 235, and a CDR-L3 comprising the amino acid sequence of SEQ ID NO: 236;
the VH domain comprises a CDR-H1 comprising the amino acid sequence of SEQ ID NO: 238, a CDR-H2 comprising the amino acid sequence of SEQ ID NO: 243, and a CDR-H3 comprising the amino acid sequence of SEQ ID NO: 233; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228: or
the VH domain comprises a CDR-H1 comprising the amino acid sequence of SEQ ID NO: 240, a CDR-H2 comprising the amino acid sequence of SEQ ID NO: 244, and a CDR-H3 comprising the amino acid sequence of SEQ ID NO: 242; and the VL domain comprises a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42.

16. (canceled)

17. The method of claim 1, wherein the second antibody or antigen-binding fragment thereof comprises a heavy chain and a light chain, wherein the heavy chain comprises a heavy chain variable (VH) domain, and the light chain comprises a light chain variable (VL) domain, and wherein the VH and VL domains of the second antibody or antigen-binding fragment thereof comprise the amino acid sequences of:
(a) SEQ ID NOs: 58 and 59, respectively;
(b) SEQ ID NOs: 247 and 248, respectively;
(c) SEQ ID NOs: 62 and 63, respectively;
(d) SEQ ID NOs: 64 and 65, respectively;
(e) SEQ ID NOs: 66 and 67, respectively;
(f) SEQ ID NOs: 68 and 69, respectively;
(g) SEQ ID NOs: 70 and 71, respectively;
(h) SEQ ID NOs: 72 and 73, respectively;
(i) SEQ ID NOs: 185 and 186, respectively;
(j) SEQ ID NOs: 245 and 246, respectively;
(k) SEQ ID NOs: 249 and 250, respectively;
(l) SEQ ID NOs: 251 and 252, respectively;
(m) SEQ ID NOs: 253 and 254, respectively;
(n) SEQ ID NOs: 255 and 256, respectively; or
(o) SEQ ID NOs: 257 and 258, respectively.

18.-24. (canceled)

25. The method of claim 1, wherein the IL-2 polypeptide comprises a sequence selected from the group consisting of SEQ ID NOs: 80, 85-155, 190-216, 297, and 354-383.

26. The method of claim 1, wherein the IL-21 polypeptide comprises an amino acid sequence that is at least 80% identical to a human IL-21 polypeptide comprising SEQ ID NO: 390.

27. (canceled)

28. The method of claim 26, wherein the IL-21 polypeptide comprises at least one amino acid substitution that reduces the isoelectric point of the IL-21 polypeptide by about 0.6 units to about 5 units relative to the human IL-21 polypeptide without the amino acid substitution.

29.-35. (canceled)

36. The method of claim 28, wherein the at least one amino acid substitution is within a region of amino acid residues positioned at S80 to T92 of the human IL-21 polypeptide comprising SEQ ID NO: 390.

37. (canceled)

38. (canceled)

39. The method of claim 26, wherein the IL-21 polypeptide comprises 4, 5, 6, 7, 8, 9, 10, 11, or 12 amino acid substitutions at positions relative to SEQ ID NO: 81 selected from the group consisting of: S80, T81, N82, A83, R85, R86, Q87, K88, H89, R90, L91, and T92 of SEQ ID NO: 390.

40.-45. (canceled)

46. The method of claim 1, wherein the IL-21 polypeptide comprises at least one amino acid substitution that reduces binding to an IL-21 receptor compared to binding by the human IL-21 polypeptide, wherein the at least one amino acid substitution is at one or more amino acid residues at positions R5, I8, R9, R11, L13, I14, I16, V17, D18, K72, K73, L74, K75, R76, K77, or K117 of SEQ ID NO: 390.

47. (canceled)

48. (canceled)

49. The method of claim 1, wherein the IL-21 polypeptide comprises an amino acid sequence at least 80% identical to an amino acid sequence selected from SEQ ID NOs: 422-491.

50. (canceled)

51. The method of claim 1, wherein the IL-21 polypeptide comprises an amino acid sequence selected from SEQ ID NOs: 422-491.

52. (canceled)

53. The method of claim 1, wherein the IL-2 fusion protein comprises four polypeptide chains, wherein:
the first polypeptide chain comprises the amino acid sequence of SEQ ID NO: 334, the second polypeptide chain comprises the amino acid sequence of SEQ ID NO: 335, the third polypeptide chain comprises the amino acid sequence of SEQ ID NO: 336, and the fourth polypeptide chain comprises the amino acid sequence of SEQ ID NO: 334;
the first polypeptide chain comprises the amino acid sequence of SEQ ID NO: 334, the second polypeptide chain comprises the amino acid sequence of SEQ ID NO: 335, the third polypeptide chain comprises the amino acid sequence of SEQ ID NO: 337, and the fourth polypeptide chain comprises the amino acid sequence of SEQ ID NO: 334;
the first polypeptide chain comprises the amino acid sequence of SEQ ID NO: 338, the second polypeptide chain comprises the amino acid sequence of SEQ ID NO: 339, the third polypeptide chain comprises the amino acid sequence of SEQ ID NO: 340, and the fourth polypeptide chain comprises the amino acid sequence of SEQ ID NO: 338; or
the first polypeptide chain comprises the amino acid sequence of SEQ ID NO: 338, the second polypeptide chain comprises the amino acid sequence of SEQ ID NO: 339, the third polypeptide chain comprises the amino acid sequence of SEQ ID NO: 341, and the fourth polypeptide chain comprises the amino acid sequence of SEQ ID NO: 338.

54. (canceled)

55. The method of claim 1, wherein the IL-21 fusion protein comprises four polypeptide chains, wherein:
the first polypeptide chain comprises the amino acid sequence of SEQ ID NO: 557, the second polypeptide chain comprises the amino acid sequence of SEQ ID NO: 558, the third polypeptide chain comprises the amino acid sequence of SEQ ID NO: 559, and the fourth polypeptide chain comprises the amino acid sequence of SEQ ID NO: 557;
the first polypeptide chain comprises the amino acid sequence of SEQ ID NO: 557, the second polypeptide chain comprises the amino acid sequence of SEQ ID NO: 558, the third polypeptide chain comprises the amino acid sequence of SEQ ID NO: 560, and the fourth polypeptide chain comprises the amino acid sequence of SEQ ID NO: 557;
the first polypeptide chain comprises the amino acid sequence of SEQ ID NO: 561, the second polypeptide chain comprises the amino acid sequence of SEQ ID NO: 562, the third polypeptide chain comprises the amino acid sequence of SEQ ID NO: 563, and the fourth polypeptide chain comprises the amino acid sequence of SEQ ID NO: 561;
the first polypeptide chain comprises the amino acid sequence of SEQ ID NO: 561, the second polypeptide chain comprises the amino acid sequence of SEQ ID NO: 562, the third polypeptide chain comprises the amino acid sequence of SEQ ID NO: 564, and the fourth polypeptide chain comprises the amino acid sequence of SEQ ID NO: 561;
the first polypeptide chain comprises the amino acid sequence of SEQ ID NO: 565, the second polypeptide chain comprises the amino acid sequence of SEQ ID NO: 566, the third polypeptide chain comprises the amino acid sequence of SEQ ID NO: 567, and the fourth polypeptide chain comprises the amino acid sequence of SEQ ID NO: 565;
the first polypeptide chain comprises the amino acid sequence of SEQ ID NO: 565, the second polypeptide chain comprises the amino acid sequence of SEQ ID NO: 566, the third polypeptide chain comprises the amino acid sequence of SEQ ID NO: 568, and the fourth polypeptide chain comprises the amino acid sequence of SEQ ID NO: 565;
the first polypeptide chain comprises the amino acid sequence of SEQ ID NO: 569, the second polypeptide chain comprises the amino acid sequence of SEQ ID NO: 570, the third polypeptide chain comprises the amino acid sequence of SEQ ID NO: 571, and the fourth polypeptide chain comprises the amino acid sequence of SEQ ID NO: 569;
the first polypeptide chain comprises the amino acid sequence of SEQ ID NO: 569, the second polypeptide chain comprises the amino acid sequence of SEQ ID NO: 570, the third polypeptide chain comprises the amino acid sequence of SEQ ID NO: 572, and the fourth polypeptide chain comprises the amino acid sequence of SEQ ID NO: 569;
the first polypeptide chain comprises the amino acid sequence of SEQ ID NO: 573, the second polypeptide chain comprises the amino acid sequence of SEQ ID NO: 574, the third polypeptide chain comprises the amino acid sequence of SEQ ID NO: 575, and the fourth polypeptide chain comprises the amino acid sequence of SEQ ID NO: 573;
the first polypeptide chain comprises the amino acid sequence of SEQ ID NO: 573, the second polypeptide chain comprises the amino acid sequence of SEQ ID NO: 574, the third polypeptide chain comprises the amino acid sequence of SEQ ID NO: 576, and the fourth polypeptide chain comprises the amino acid sequence of SEQ ID NO: 573;
the first polypeptide chain comprises the amino acid sequence of SEQ ID NO: 577, the second polypeptide chain comprises the amino acid sequence of SEQ ID NO: 578, the third polypeptide chain comprises the amino acid sequence of SEQ ID NO: 579, and the fourth polypeptide chain comprises the amino acid sequence of SEQ ID NO: 577;
the first polypeptide chain comprises the amino acid sequence of SEQ ID NO: 577, the second polypeptide chain comprises the amino acid sequence of SEQ ID NO: 578, the third polypeptide chain comprises the amino acid sequence of SEQ ID NO: 580, and the fourth polypeptide chain comprises the amino acid sequence of SEQ ID NO: 577;
the first polypeptide chain comprises the amino acid sequence of SEQ ID NO: 581, the second polypeptide chain comprises the amino acid sequence of SEQ ID NO: 582, the third polypeptide chain comprises the amino acid sequence of SEQ ID NO: 583, and the fourth polypeptide chain comprises the amino acid sequence of SEQ ID NO: 581;
the first polypeptide chain comprises the amino acid sequence of SEQ ID NO: 581, the second polypeptide chain comprises the amino acid sequence of SEQ ID NO: 582, the third polypeptide chain comprises the amino acid sequence of SEQ ID NO: 584, and the fourth polypeptide chain comprises the amino acid sequence of SEQ ID NO: 581;
the first polypeptide chain comprises the amino acid sequence of SEQ ID NO: 585, the second polypeptide chain comprises the amino acid sequence of SEQ ID NO: 586, the third polypeptide chain comprises the amino acid sequence of SEQ ID NO: 587, and the fourth polypeptide chain comprises the amino acid sequence of SEQ ID NO: 585;
the first polypeptide chain comprises the amino acid sequence of SEQ ID NO: 585, the second polypeptide chain comprises the amino acid sequence of SEQ ID NO: 586, the third polypeptide chain comprises the amino acid sequence of SEQ ID NO: 588, and the fourth polypeptide chain comprises the amino acid sequence of SEQ ID NO: 585;
the first polypeptide chain comprises the amino acid sequence of SEQ ID NO: 589, the second polypeptide chain comprises the amino acid sequence of SEQ ID NO: 590, the third polypeptide chain comprises the amino acid sequence of SEQ ID NO: 591, and the fourth polypeptide chain comprises the amino acid sequence of SEQ ID NO: 589; or
the first polypeptide chain comprises the amino acid sequence of SEQ ID NO: 589, the second polypeptide chain comprises the amino acid sequence of SEQ ID NO: 590, the third polypeptide chain comprises the amino acid sequence of SEQ ID NO: 592, and the fourth polypeptide chain comprises the amino acid sequence of SEQ ID NO: 589.

56.-61. (canceled)

62. The method of claim 1, wherein the first antibody or antigen binding fragment thereof and the second antibody or antigen binding fragment thereof each independently targets CD8ab or CD8b.

63. (canceled)

64. (canceled)

65. A kit comprising one or more containers comprising (a) an IL-21 fusion protein comprising (i) a first antibody or antigen-binding fragment thereof that specifically binds to human CD8, and (ii) an IL-21 polypeptide; and (b) an TL-2 fusion protein comprising (i) a second antibody or antigen-binding fragment thereof that specifically binds human CD8, and (ii) an IL-2 polypeptide, optionally a pharmaceutically acceptable carrier, optionally instructions for administering the IL-21 fusion protein and the IL-2 fusion protein concurrently, sequentially, or simultaneously to a subject in need thereof.

66. (canceled)

67. (canceled)

68. A composition comprising (a) an IL-21 fusion protein comprising (i) a first antibody or antigen-binding fragment thereof that specifically binds to human CD8, and (ii) an IL-21 polypeptide; and (b) an IL-2 fusion protein comprising (i) a second antibody or antigen-binding fragment thereof that specifically binds human CD8, and (ii) an IL-2 polypeptide.

69. (canceled)
